SEQUENCE LISTING (729 sequences)

SEQ ID NO: 1 - protein, 19 aa, Homo sapiens
VPSTPPTPSPSTPPTPSPS

SEQ ID NO: 2 - protein, 6 aa, Homo sapiens
VPPPPP

SEQ ID NO: 3 - protein, 58 aa, Homo sapiens
ESPKAQASSVPTAQPQAEGSLAKATTAPATTRNTGRGGEEKKKEKEKEEQEERETKTP

SEQ ID NO: 4 - protein, 15 aa, Homo sapiens
EPKSCDKTHTCPPCP

SEQ ID NO: 5 - protein, 12 aa, Homo sapiens
ERKCCVECPPCP

SEQ ID NO: 6 - protein, 62 aa, Homo sapiens
ELKTPLGDTTHTCPRCPEPKSCDTPPPCPRCPEPKSCDTPPPCPRCPEPKSCDTPPPCPRCP

SEQ ID NO: 7 - protein, 12 aa, Homo sapiens
ESKYGPPCPSCP

SEQ ID NO: 8 - protein, 12 aa, Homo sapiens
ESKYGPPCPPCP

SEQ ID NO: 9 - protein, 4 aa, synthetic construct
  REGION 1..4: note = Description of Artificial Sequence: Synthetic peptide
CPPC

SEQ ID NO: 10 - protein, 4 aa, synthetic construct
  REGION 1..4: note = Description of Artificial Sequence: Synthetic peptide
CPSC

SEQ ID NO: 11 - protein, 4 aa, synthetic construct
  REGION 1..4: note = Description of Artificial Sequence: Synthetic peptide
CPRC

SEQ ID NO: 12 - protein, 4 aa, synthetic construct
  REGION 1..4: note = Description of Artificial Sequence: Synthetic peptide
SPPC

SEQ ID NO: 13 - protein, 4 aa, synthetic construct
  REGION 1..4: note = Description of Artificial Sequence: Synthetic peptide
CPPS

SEQ ID NO: 14 - protein, 4 aa, synthetic construct
  REGION 1..4: note = Description of Artificial Sequence: Synthetic peptide
SPPS

SEQ ID NO: 15 - protein, 8 aa, synthetic construct
  REGION 1..8: note = Description of Artificial Sequence: Synthetic peptide
DKTHTCAA

SEQ ID NO: 16 - protein, 11 aa, synthetic construct
  REGION 1..11: note = Description of Artificial Sequence: Synthetic peptide
DKTHTCPPCPA

SEQ ID NO: 17 - protein, 18 aa, synthetic construct
  REGION 1..18: note = Description of Artificial Sequence: Synthetic peptide
DKTHTCPPCPATCPPCPA

SEQ ID NO: 18 - protein, 25 aa, synthetic construct
  REGION 1..25: note = Description of Artificial Sequence: Synthetic peptide
DKTHTCPPCPATCPPCPATCPPCPA

SEQ ID NO: 19 - protein, 30 aa, synthetic construct
  REGION 1..30: note = Description of Artificial Sequence: Synthetic polypeptide
DKTHTCPPCPAGKPTLYNSLVMSDTAGTCY

SEQ ID NO: 20 - protein, 31 aa, synthetic construct
  REGION 1..31: note = Description of Artificial Sequence: Synthetic polypeptide
DKTHTCPPCPAGKPTHVNVSVVMAEVDGTCY

SEQ ID NO: 21 - protein, 15 aa, synthetic construct
  REGION 1..15: note = Description of Artificial Sequence: Synthetic peptide
DKTHTCCVECPPCPA

SEQ ID NO: 22 - protein, 26 aa, synthetic construct
  REGION 1..26: note = Description of Artificial Sequence: Synthetic peptide
DKTHTCPRCPEPKSCDTPPPCPRCPA

SEQ ID NO: 23 - protein, 11 aa, synthetic construct
  REGION 1..11: note = Description of Artificial Sequence: Synthetic peptide
DKTHTCPSCPA

SEQ ID NO: 24 - protein, 5 aa, synthetic construct
  REGION 1..5: note = Description of Artificial Sequence: Synthetic peptide
ADAAP

SEQ ID NO: 25 - protein, 11 aa, synthetic construct
  REGION 1..11: note = Description of Artificial Sequence: Synthetic peptide
ADAAPTVSIFP

SEQ ID NO: 26 - protein, 12 aa, synthetic construct
  REGION 1..12: note = Description of Artificial Sequence: Synthetic peptide
ADAAPTVSIFPP

SEQ ID NO: 27 - protein, 6 aa, synthetic construct
  REGION 1..6: note = Description of Artificial Sequence: Synthetic peptide
AKTTAP

SEQ ID NO: 28 - protein, 13 aa, synthetic construct
  REGION 1..13: note = Description of Artificial Sequence: Synthetic peptide
AKTTAPSVYPLAP

SEQ ID NO: 29 - protein, 17 aa, synthetic construct
  REGION 1..17: note = Description of Artificial Sequence: Synthetic peptide
AKTTPKLEEGEFSEARV

SEQ ID NO: 30 - protein, 9 aa, synthetic construct
  REGION 1..9: note = Description of Artificial Sequence: Synthetic peptide
AKTTPKLGG

SEQ ID NO: 31 - protein, 6 aa, synthetic construct
  REGION 1..6: note = Description of Artificial Sequence: Synthetic peptide
AKTTPP

SEQ ID NO: 32 - protein, 13 aa, synthetic construct
  REGION 1..13: note = Description of Artificial Sequence: Synthetic peptide
AKTTPPSVTPLAP

SEQ ID NO: 33 - protein, 6 aa, synthetic construct
  REGION 1..6: note = Description of Artificial Sequence: Synthetic peptide
ASTKGP

SEQ ID NO: 34 - protein, 13 aa, synthetic construct
  REGION 1..13: note = Description of Artificial Sequence: Synthetic peptide
ASTKGPSVFPLAP

SEQ ID NO: 35 - protein, 26 aa, synthetic construct
  REGION 1..26: note = Description of Artificial Sequence: Synthetic peptide
ASTKGPSVFPLAPASTKGPSVFPLAP

SEQ ID NO: 36 - protein, 14 aa, synthetic construct
  REGION 1..14: note = Description of Artificial Sequence: Synthetic peptide
EGKSSGSGSESKST

SEQ ID NO: 37 - protein, 15 aa, synthetic construct
  REGION 1..15: note = Description of Artificial Sequence: Synthetic peptide
GEGESGEGESGEGES

SEQ ID NO: 38 - protein, 20 aa, synthetic construct
  REGION 1..20: note = Description of Artificial Sequence: Synthetic peptide
GEGESGEGESGEGESGEGES

SEQ ID NO: 39 - protein, 15 aa, synthetic construct
  REGION 1..15: note = Description of Artificial Sequence: Synthetic peptide
GEGGSGEGGSGEGGS

SEQ ID NO: 40 - protein, 15 aa, synthetic construct
  REGION 1..15: note = Description of Artificial Sequence: Synthetic peptide
GENKVEYAPALMALS

SEQ ID NO: 41 - protein, 15 aa, synthetic construct
  REGION 1..15: note = Description of Artificial Sequence: Synthetic peptide
GGEGSGGEGSGGEGS

SEQ ID NO: 42 - protein, 15 aa, synthetic construct
  REGION 1..15: note = Description of Artificial Sequence: Synthetic peptide
GGGESGGEGSGEGGS

SEQ ID NO: 43 - protein, 15 aa, synthetic construct
  REGION 1..15: note = Description of Artificial Sequence: Synthetic peptide
GGGESGGGESGGGES

SEQ ID NO: 44 - protein, 50 aa, synthetic construct
  REGION 1..50: note = Description of Artificial Sequence: Synthetic polypeptide
  VARIANT 1..50: note = MISC_FEATURE - This sequence may encompass 1-10 GGGGS repeating units
GGGGSGGGGSGGGGSGGGGSGGGGSGGGGSGGGGSGGGGSGGGGSGGGGS

SEQ ID NO: 45 - protein, 10 aa, synthetic construct
  REGION 1..10: note = Description of Artificial Sequence: Synthetic peptide
GGGGSGGGGS

SEQ ID NO: 46 - protein, 15 aa, synthetic construct
  REGION 1..15: note = Description of Artificial Sequence: Synthetic peptide
GGGGSGGGGSGGGGS

SEQ ID NO: 47 - protein, 20 aa, synthetic construct
  REGION 1..20: note = Description of Artificial Sequence: Synthetic peptide
GGGGSGGGGSGGGGSGGGGS

SEQ ID NO: 48 - protein, 15 aa, synthetic construct
  REGION 1..15: note = Description of Artificial Sequence: Synthetic peptide
GGGKSGGGKSGGGKS

SEQ ID NO: 49 - protein, 15 aa, synthetic construct
  REGION 1..15: note = Description of Artificial Sequence: Synthetic peptide
GGGKSGGKGSGKGGS

SEQ ID NO: 50 - protein, 15 aa, synthetic construct
  REGION 1..15: note = Description of Artificial Sequence: Synthetic peptide
GGKGSGGKGSGGKGS

SEQ ID NO: 51 - protein, 5 aa, synthetic construct
  REGION 1..5: note = Description of Artificial Sequence: Synthetic peptide
GGSGG

SEQ ID NO: 52 - protein, 9 aa, synthetic construct
  REGION 1..9: note = Description of Artificial Sequence: Synthetic peptide
GGSGGGGSG

SEQ ID NO: 53 - protein, 13 aa, synthetic construct
  REGION 1..13: note = Description of Artificial Sequence: Synthetic peptide
GGSGGGGSGGGGS

SEQ ID NO: 54 - protein, 15 aa, synthetic construct
  REGION 1..15: note = Description of Artificial Sequence: Synthetic peptide
GHEAAAVMQVQYPAS

SEQ ID NO: 55 - protein, 15 aa, synthetic construct
  REGION 1..15: note = Description of Artificial Sequence: Synthetic peptide
GKGGSGKGGSGKGGS

SEQ ID NO: 56 - protein, 15 aa, synthetic construct
  REGION 1..15: note = Description of Artificial Sequence: Synthetic peptide
GKGKSGKGKSGKGKS

SEQ ID NO: 57 - protein, 20 aa, synthetic construct
  REGION 1..20: note = Description of Artificial Sequence: Synthetic peptide
GKGKSGKGKSGKGKSGKGKS

SEQ ID NO: 58 - protein, 15 aa, synthetic construct
  REGION 1..15: note = Description of Artificial Sequence: Synthetic peptide
GKPGSGKPGSGKPGS

SEQ ID NO: 59 - protein, 20 aa, synthetic construct
  REGION 1..20: note = Description of Artificial Sequence: Synthetic peptide
GKPGSGKPGSGKPGSGKPGS

SEQ ID NO: 60 - protein, 15 aa, synthetic construct
  REGION 1..15: note = Description of Artificial Sequence: Synthetic peptide
GPAKELTPLKEAKVS

SEQ ID NO: 61 - protein, 12 aa, synthetic construct
  REGION 1..12: note = Description of Artificial Sequence: Synthetic peptide
GSAGSAAGSGEF

SEQ ID NO: 62 - protein, 14 aa, synthetic construct
  REGION 1..14: note = Description of Artificial Sequence: Synthetic peptide
IRPRAIGGSKPRVA

SEQ ID NO: 63 - protein, 18 aa, synthetic construct
  REGION 1..18: note = Description of Artificial Sequence: Synthetic peptide
KESGSVSSEQLAQFRSLD

SEQ ID NO: 64 - protein, 15 aa, synthetic construct
  REGION 1..15: note = Description of Artificial Sequence: Synthetic peptide
KTTPKLEEGEFSEAR

SEQ ID NO: 65 - protein, 6 aa, synthetic construct
  REGION 1..6: note = Description of Artificial Sequence: Synthetic peptide
QPKAAP

SEQ ID NO: 66 - protein, 13 aa, synthetic construct
  REGION 1..13: note = Description of Artificial Sequence: Synthetic peptide
QPKAAPSVTLFPP

SEQ ID NO: 67 - protein, 27 aa, synthetic construct
  REGION 1..27: note = Description of Artificial Sequence: Synthetic peptide
RADAAAAGGGGSGGGGSGGGGSGGGGS

SEQ ID NO: 68 - protein, 12 aa, synthetic construct
  REGION 1..12: note = Description of Artificial Sequence: Synthetic peptide
RADAAAAGGPGS

SEQ ID NO: 69 - protein, 6 aa, synthetic construct
  REGION 1..6: note = Description of Artificial Sequence: Synthetic peptide
RADAAP

SEQ ID NO: 70 - protein, 9 aa, synthetic construct
  REGION 1..9: note = Description of Artificial Sequence: Synthetic peptide
RADAAPTVS

SEQ ID NO: 71 - protein, 6 aa, synthetic construct
  REGION 1..6: note = Description of Artificial Sequence: Synthetic peptide
SAKTTP

SEQ ID NO: 72 - protein, 18 aa, synthetic construct
  REGION 1..18: note = Description of Artificial Sequence: Synthetic peptide
SAKTTPKLEEGEFSEARV

SEQ ID NO: 73 - protein, 10 aa, synthetic construct
  REGION 1..10: note = Description of Artificial Sequence: Synthetic peptide
SAKTTPKLGG

SEQ ID NO: 74 - protein, 14 aa, synthetic construct
  REGION 1..14: note = Description of Artificial Sequence: Synthetic peptide
STAGDTHLGGEDFD

SEQ ID NO: 75 - protein, 5 aa, synthetic construct
  REGION 1..5: note = Description of Artificial Sequence: Synthetic peptide
TVAAP

SEQ ID NO: 76 - protein, 12 aa, synthetic construct
  REGION 1..12: note = Description of Artificial Sequence: Synthetic peptide
TVAAPSVFIFPP

SEQ ID NO: 77 - protein, 24 aa, synthetic construct
  REGION 1..24: note = Description of Artificial Sequence: Synthetic peptide
TVAAPSVFIFPPTVAAPSVFIFPP

SEQ ID NO: 78 - protein, 119 aa, synthetic construct [sequence omitted]

SEQ ID NO: 79 - protein, 106 aa, synthetic construct [sequence omitted]

SEQ ID NO: 80 - protein, 125 aa, synthetic construct
  REGION 1..125: note = Description of Artificial Sequence: Synthetic polypeptide
EVQLVESGGGLVQPKGSLKLSCAASGFTFNTYAMNWVRQAPGKGLEWVARIRSKYNNYATYYADSVKDRFTISRDDSQSILYLQMNNLKTEDTAMYYCVRHGNFGNSYVSWFAYWGQGTLVTVSA

SEQ ID NO: 81 - protein, 109 aa, synthetic construct
  REGION 1..109: note = Description of Artificial Sequence: Synthetic polypeptide
QAVVTQESALTTSPGETVTLTCRSSTGAVTTSNYANWVQEKPDHLFTGLIGGTNKRAPGVPARFSGSLIGDKAALTITGAQTEDEAIYFCALWYSNLWVFGGGTKLTVL

SEQ ID NO: 82 - protein, 119 aa, synthetic construct
  REGION 1..119: note = Description of Artificial Sequence: Synthetic polypeptide
QVQLQQSGAELARPGASVKMSCKASGYTFTSYTMHWVKQRPGQGLEWIGYINPSSGYTKYNQKFKDKATLTADKSSSTAYMQLSSLTSEDSAVYYCARWQDYDVYFDYWGQGTTLTVSS

SEQ ID NO: 83 - protein, 106 aa, synthetic construct
  REGION 1..106: note = Description of Artificial Sequence: Synthetic polypeptide
QIVLSQSPAILSASPGEKVTMTCRASSSVSYMHWYQQKPGSSPKPWIYATSNLASGVPARFSGSGSGTSYSLTISRVEAEDAATYYCQQWSSNPPTFGGGTKLETK

SEQ ID NO: 84 - protein, 106 aa, synthetic construct
  REGION 1..106: note = Description of Artificial Sequence: Synthetic polypeptide
QIVLTQSPAIMSASPGEKVTMTCRASSSVSYMNWYQQKSGTSPKRWIYDTSKVASGVPYRFSGSGSGTSYSLTISSMEAEDAATYYCQQWSSNPLTFGSGTKLEIN

SEQ ID NO: 85 - protein, 119 aa, synthetic construct [sequence omitted]

SEQ ID NO: 86 - protein, 106 aa, synthetic construct [sequence omitted]

SEQ ID NO: 87 - protein, 118 aa, synthetic construct [sequence omitted]

SEQ ID NO: 88 - protein, 108 aa, synthetic construct [sequence omitted]

SEQ ID NO: 89 - protein, 124 aa, synthetic construct
  REGION 1..124: note = Description of Artificial Sequence: Synthetic polypeptide
EVQLVESGGGLVQPGGSLRLSCAASGFTFSTYAMNWVRQAPGKGLEWVGRIRSKYNNYATYYADSVKDRFISRDDSKNSLYLQMNSLKTEDTAVYYCVRHGNFGNSYVSWFAYWGQGTLVTVSS

SEQ ID NO: 90 - protein, 107 aa, synthetic construct
  REGION 1..107: note = Description of Artificial Sequence: Synthetic polypeptide
QAVVTQEPSLTVSPGGTVTLTCRSSTGAVTTSNYANWVQQKPGQAPRGLIGGTNKRAPWTPARFSGSLLGGKAALIGAQAEDEADYYCALWYSNLWVFGGGTKLTVL

SEQ ID NO: 91 - protein, 119 aa, synthetic construct
  REGION 1..119: note = Description of Artificial Sequence: Synthetic polypeptide
DIKLQQSGAELARPGASVKMSCKTSGYTFTRYTMHWVKQRPGQGLEWIGYINPSRGYTNYNQKFKDKATLTTDKSSSTAYMQLSSLTSEDSAVYYCARYYDDHYCLDYWGQGTTLTVSS

SEQ ID NO: 92 - protein, 105 aa, synthetic construct
  REGION 1..105: note = Description of Artificial Sequence: Synthetic polypeptide
DIQLTQSPAIMSASPGEKVTMTCRASSSVSYMNWYQQKSGTSPKRWIYDTSKVASGVPYRFSGSGSGTSYSLISSMEAEDAATYYCQQWSSNPLTFGAGTKLELK

SEQ ID NO: 93 - protein, 124 aa, synthetic construct
  REGION 1..124: note = Description of Artificial Sequence: Synthetic polypeptide
EVQLVESGGGLVQPGGSLRLSCAASGFTFNTYAMNWVRQAPGKGLEWVARIRSKYNNYATYYADSVKDRFISRDDSKNSLYLQMNSLKTEDTAVYYCVRHGNFGNSYVSWFAYWGQGTLVTVSS

SEQ ID NO: 94 - protein, 125 aa, synthetic construct
  REGION 1..125: note = Description of Artificial Sequence: Synthetic polypeptide
EVKLLESGGGLVQPKGSLKLSCAASGFTFNTYAMNWVRQAPGKGLEWVARIRSKYNNYATYYADSVKDRFTISRDDSQSILYLQMNNLKTEDTAMYYCVRHGNFGNSYVSWFAYWGQGTLVTVSA

SEQ ID NO: 95 - protein, 125 aa, synthetic construct
  REGION 1..125: note = Description of Artificial Sequence: Synthetic polypeptide
EVQLVESGGGLVQPGGSLKLSCAASGFTFNSYAMNWVRQAPGKGLEWVARIRSKYNNYATYYADSVKGRFTISRDDSKNTAYLQMNNLKTEDTAVYYCVRHGNFGNSYVSWWAYWGQGTLVTVSS

SEQ ID NO: 96 - protein, 109 aa, synthetic construct
  REGION 1..109: note = Description of Artificial Sequence: Synthetic polypeptide
QTVVTQEPSLTVSPGGTVTLTCGSSTGAVTSGNYPNWVQQKPGQAPRGLIGGTKFLAPGTPQRFSGSLLGGKAALTLSGVQPEDEAEYYCVLWYSNRWVFGGGTKLTVL

SEQ ID NO: 97 - protein, 125 aa, synthetic construct
  REGION 1..125: note = Description of Artificial Sequence: Synthetic polypeptide
EVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSS

SEQ ID NO: 98 - protein, 109 aa, synthetic construct
  REGION 1..109: note = Description of Artificial Sequence: Synthetic polypeptide
QTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVL

SEQ ID NO: 99 - protein, 119 aa, synthetic construct
  REGION 1..119: note = Description of Artificial Sequence: Synthetic polypeptide
QVQLVQSGAEVKKPGASVKVSCKASGYTFTRYTMHWVRQAPGQGLEWMGYINPSRGYTNYNQKFKDRVTMTTDTSISTAYMELSRLRSDDTAVYYCARYYDDHYCLDYWGQGTLVTVSS

SEQ ID NO: 100 - protein, 106 aa, synthetic construct
  REGION 1..106: note = Description of Artificial Sequence: Synthetic polypeptide
EIVLTQSPATLSLSPGERATLSCSASSSVSYMNWYQQKPGQAPRLLIYDTSKLASGVPAHFRGSGSGTDFTLTISSLEPEDFAVYYCQQWSSNPFTFGQGTKVEIK

SEQ ID NO: 101 - protein, 125 aa, synthetic construct
  REGION 1..125: note = Description of Artificial Sequence: Synthetic polypeptide
EVQLVESGGGLVQPGGSLRLSCAASGFTFSTYAMNWVRQAPGKGLEWVSRIRSKYNNYATYYADSVKDRFTISRDDSKNTLYLQMNSLRAEDTAVYYCARHGNFGNSYVSWFAYWGQGTMVTVSS

SEQ ID NO: 102 - protein, 109 aa, synthetic construct
  REGION 1..109: note = Description of Artificial Sequence: Synthetic polypeptide
QAVVTQEPSLTVSPGGTVTLTCRSSTGAVTTSNYANWVQQKPGQAPRGLIGGTNKRAPGTPARFSGSLLGGKAALTLSGVQPEDEAEYYCALWYSNLWVFGGGTKLTVL

SEQ ID NO: 103 - protein, 125 aa, synthetic construct
  REGION 1..125: note = Description of Artificial Sequence: Synthetic polypeptide
EVQLVESGGGLVQPGGSLRLSCAASGFTFNTYAMNWVRQAPGKGLEWVGRIRSKYNNYATYYADSVKDRFTISRDDSKNSLYLQMNSLKTEDTAVYYCVRHGNFGNSYVSWFAYWGQGTLVTVSS

SEQ ID NO: 104 - protein, 109 aa, synthetic construct
  REGION 1..109: note = Description of Artificial Sequence: Synthetic polypeptide
QAVVTQEPSLTVSPGGTVTLTCRSSTGAVTTSNYANWVQEKPGQAPRGLIGGTNKRAPWTPARFSGSLLGGKAALTITGAQAEDEADYYCALWYSNLWVFGGGTKLTVL

SEQ ID NO: 105 - protein, 125 aa, synthetic construct
  REGION 1..125: note = Description of Artificial Sequence: Synthetic polypeptide
EVQLVESGGGLVQPGGSLRLSCAASGFTFNTYAMNWVRQAPGKGLEWVGRIRSKYNNYATYYADSVKGRFTISRDDSKNTLYLQMNSLRAEDTAVYYCVRHGNFGNSYVSWFAYWGQGTLVTVSS

SEQ ID NO: 106 - protein, 109 aa, synthetic construct
  REGION 1..109: note = Description of Artificial Sequence: Synthetic polypeptide
QAVVTQEPSLTVSPGGTVTLTCGSSTGAVTTSNYANWVQQKPGQAPRGLIGGTNKRAPGVPARFSGSLLGGKAALTLSGAQPEDEAEYYCALWYSNLWVFGGGTKLTVL

SEQ ID NO: 107 - protein, 125 aa, synthetic construct
  REGION 1..125: note = Description of Artificial Sequence: Synthetic polypeptide
EVQLVESGGGLVQPGGSLRLSCAASGFTFSTYAMNWVRQAPGKGLEWVGRIRSKYNNYATYYADSVKGRFTISRDDSKNTLYLQMNSLRAEDTAVYYCVRHGNFGDSYVSWFAYWGQGTLVTVSS

SEQ ID NO: 108 - protein, 109 aa, synthetic construct
  REGION 1..109: note = Description of Artificial Sequence: Synthetic polypeptide
QAVVTQEPSLTVSPGGTVTLTCGSSTGAVTTSNYANWVQQKPGKSPRGLIGGTNKRAPGVPARFSGSLLGGKAALTISGAQPEDEADYYCALWYSNHWVFGGGTKLTVL

SEQ ID NO: 109 - protein, 101 aa, synthetic construct
  REGION 1..101: note = Description of Artificial Sequence: Synthetic polypeptide
DIQMTQSPSSLSASVGDRVTITCSASSSVSYMNWYQQTPGKAPKRWIYDTSKLASGVPSRFSGSGSGTDYTFTISSLQPEDIATYYCQQWSSNPFTFGQGT

SEQ ID NO: 110 - protein, 119 aa, synthetic construct
  REGION 1..119: note = Description of Artificial Sequence: Synthetic polypeptide
QVQLVQSGGGVVQPGRSLRLSCKASGYTFTRYTMHWVRQAPGKGLEWIGYINPSRGYTNYNQKVKDRFTISRDNSKNTAFLQMDSLRPEDTGVYFCARYYDDHYSLDYWGQGTPVTVSS

SEQ ID NO: 111 - protein, 122 aa, synthetic construct
  REGION 1..122: note = Description of Artificial Sequence: Synthetic polypeptide
EVQLQQSGPELVKPGASMKISCKASGYSFTGYTMNWVKQSHGKNLEWMGLINPYKGVSTYNQKFKDKATLTVDKSSSTAYMELLSLTSEDSAVYYCARSGYYGDSDWYFDVWGQGTTLTVFS

SEQ ID NO: 112 - protein, 107 aa, synthetic construct
  REGION 1..107: note = Description of Artificial Sequence: Synthetic polypeptide
DIQMTQTTSSLSASLGDRVTISCRASQDIRNYLNWYQQKPDGTVKLLIYYTSRLHSGVPSKFSGSGSGTDYSLTISNLEQEDIATYFCQQGNTLPWTFAGGTKLEIK

SEQ ID NO: 113 - protein, 125 aa, synthetic construct
  REGION 1..125: note = Description of Artificial Sequence: Synthetic polypeptide
EVQLVESGGGLVQPGGSLKLSCAASGFTFNTYAMNWVRQASGKGLEWVGRIRSKYNNYATYYADSVKDRFTISRDDSKSTLYLQMNSLKTEDTAVYYCVRHGNFGNSYVSWFAYWGQGTLVTVSS

SEQ ID NO: 114 - protein, 109 aa, synthetic construct
  REGION 1..109: note = Description of Artificial Sequence: Synthetic polypeptide
QAVVTQEPSLTVSPGGTVTLTCRSSTGAVTTSNYANWVQQKPGQAPRGLIGGTNKRAPWTPARFSGSLLGDKAALTLSGAQPEDEAEYFCALWYSNLWVFGGGTKLTVL

SEQ ID NO: 115 - protein, 254 aa, synthetic construct
  REGION 1..254: note = Description of Artificial Sequence: Synthetic polypeptide
EVQLVESGGGLVQPGGSLKLSCAASGFTFNTYAMNWVRQASGKGLEWVGRIRSKYNNYATYYADSVKDRFTISRDDSKSTLYLQMNSLKTEDTAVYYCVRHGNFGNSYVSWFAYWGQGTLVTVSSGGGGSGGGGSGGGGSGGGGSQAVVTQEPSLTVSPGGTVTLTCRSSTGAVTTSNYANWVQQKPGQAPRGLIGGTNKRAPWTPARFSGSLLGDKAALTLSGAQPEDEAEYFCALWYSNLWVFGGGTKLTVL

SEQ ID NO: 116 - protein, 254 aa, synthetic construct
  REGION 1..254: note = Description of Artificial Sequence: Synthetic polypeptide
EVQLVESGGGLVQPGGSLRLSCAASGFTFSTYAMNWVRQAPGKGLEWVGRIRSKYNNYATYYADSVKGRFTISRDDSKNTLYLQMNSLRAEDTAVYYCVRHGNFGDSYVSWFAYWGQGTLVTVSSGKPGSGKPGSGKPGSGKPGSQAVVTQEPSLTVSPGGTVTLTCGSSTGAVTTSNYANWVQQKPGKSPRGLIGGTNKRAPGVPARFSGSLLGGKAALTISGAQPEDEADYYCALWYSNHWVFGGGTKLTVL

SEQ ID NO: 117 - protein, 125 aa, synthetic construct
  REGION 1..125: note = Description of Artificial Sequence: Synthetic polypeptide
EVQLVESGGGLVQPGGSLRLSCAASGFTFSTYAMNWVRQAPGKGLEWVGRIRSKANNYATYYADSVKGRFTISRDDSKNTLYLQMNSLRAEDTAVYYCVRHGNFGDSYVSWFAYWGQGTLVTVSS

SEQ ID NO: 118 - protein, 254 aa, synthetic construct
  REGION 1..254: note = Description of Artificial Sequence: Synthetic polypeptide
EVQLVESGGGLVQPGGSLRLSCAASGFTFSTYAMNWVRQAPGKGLEWVGRIRSKANNYATYYADSVKGRFTISRDDSKNTLYLQMNSLRAEDTAVYYCVRHGNFGDSYVSWFAYWGQGTLVTVSSGKPGSGKPGSGKPGSGKPGSQAVVTQEPSLTVSPGGTVTLTCGSSTGAVTTSNYANWVQQKPGKSPRGLIGGTNKRAPGVPARFSGSLLGGKAALTISGAQPEDEADYYCALWYSNHWVFGGGTKLTVL

SEQ ID NO: 119 - protein, 125 aa, synthetic construct
  REGION 1..125: note = Description of Artificial Sequence: Synthetic polypeptide
EVQLVESGGGLVQPGGSLRLSCAASGFTFSTYAMNWVRQAPGKGLEWVGRIRSKYNNYATYYADSVKGRFTISRDDSKNTLYLQMNSLRAEDTAVYYCVRHGNFGDEYVSWFAYWGQGTLVTVSS

SEQ ID NO: 120 - protein, 254 aa, synthetic construct
  REGION 1..254: note = Description of Artificial Sequence: Synthetic polypeptide
EVQLVESGGGLVQPGGSLRLSCAASGFTFSTYAMNWVRQAPGKGLEWVGRIRSKYNNYATYYADSVKGRFTISRDDSKNTLYLQMNSLRAEDTAVYYCVRHGNFGDEYVSWFAYWGQGTLVTVSSGKPGSGKPGSGKPGSGKPGSQAVVTQEPSLTVSPGGTVTLTCGSSTGAVTTSNYANWVQQKPGKSPRGLIGGTNKRAPGVPARFSGSLLGGKAALTISGAQPEDEADYYCALWYSNHWVFGGGTKLTVL

SEQ ID NO: 121 - protein, 125 aa, synthetic construct
  REGION 1..125: note = Description of Artificial Sequence: Synthetic polypeptide
EVQLVESGGGLVQPGGSLRLSCAASGFTFSTYAMNWVRQAPGKGLEWVGRIRSKYNNYATYYADSVKGRFTISRDDSKNTLYLQMNSLRAEDTAVYYCVRHGNFGDPYVSWFAYWGQGTLVTVSS

SEQ ID NO: 122 - protein, 254 aa, synthetic construct
  REGION 1..254: note = Description of Artificial Sequence: Synthetic polypeptide
EVQLVESGGGLVQPGGSLRLSCAASGFTFSTYAMNWVRQAPGKGLEWVGRIRSKYNNYATYYADSVKGRFTISRDDSKNTLYLQMNSLRAEDTAVYYCVRHGNFGDPYVSWFAYWGQGTLVTVSSGKPGSGKPGSGKPGSGKPGSQAVVTQEPSLTVSPGGTVTLTCGSSTGAVTTSNYANWVQQKPGKSPRGLIGGTNKRAPGVPARFSGSLLGGKAALTISGAQPEDEADYYCALWYSNHWVFGGGTKLTVL

SEQ ID NO: 123 - protein, 125 aa, synthetic construct
  REGION 1..125: note = Description of Artificial Sequence: Synthetic polypeptide
EVQLVESGGGLVQPGGSLRLSCAASGFTFSTYAMNWVRQAPGKGLEWVGRIRSKYNNYATYYADSVKGRFTISRDDSKNTLYLQMNSLRAEDTAVYYCVRHGNFGDSYVSWFDYWGQGTLVTVSS

SEQ ID NO: 124 - protein, 254 aa, synthetic construct
  REGION 1..254: note = Description of Artificial Sequence: Synthetic polypeptide
EVQLVESGGGLVQPGGSLRLSCAASGFTFSTYAMNWVRQAPGKGLEWVGRIRSKYNNYATYYADSVKGRFTISRDDSKNTLYLQMNSLRAEDTAVYYCVRHGNFGDSYVSWFDYWGQGTLVTVSSGKPGSGKPGSGKPGSGKPGSQAVVTQEPSLTVSPGGTVTLTCGSSTGAVTTSNYANWVQQKPGKSPRGLIGGTNKRAPGVPARFSGSLLGGKAALTISGAQPEDEADYYCALWYSNHWVFGGGTKLTVL

SEQ ID NO: 125 - protein, 125 aa, synthetic construct
  REGION 1..125: note = Description of Artificial Sequence: Synthetic polypeptide
EVQLVESGGGLVQPGGSLRLSCAASGFTFSTYAMSWVRQAPGKGLEWVGRIRSKYNNYATYYADSVKGRFTISRDDSKNTLYLQMNSLRAEDTAVYYCVRHGNFGDSYVSWFAYWGQGTLVTVSS

SEQ ID NO: 126 - protein, 254 aa, synthetic construct
  REGION 1..254: note = Description of Artificial Sequence: Synthetic polypeptide
EVQLVESGGGLVQPGGSLRLSCAASGFTFSTYAMSWVRQAPGKGLEWVGRIRSKYNNYATYYADSVKGRFTISRDDSKNTLYLQMNSLRAEDTAVYYCVRHGNFGDSYVSWFAYWGQGTLVTVSSGKPGSGKPGSGKPGSGKPGSQAVVTQEPSLTVSPGGTVTLTCGSSTGAVTTSNYANWVQQKPGKSPRGLIGGTNKRAPGVPARFSGSLLGGKAALTISGAQPEDEADYYCALWYSNHWVFGGGTKLTVL

SEQ ID NO: 127 - protein, 257 aa, synthetic construct
  REGION 1..257: note = Description of Artificial Sequence: Synthetic polypeptide
EVQLVESGGGLVQPGGSLRLSCAASGFTFNTYAMNWVRQAPGKGLEWVGRIRSKYNNYATYYADSVKGRFTISRDDSKNTLYLQMNSLRAEDTAVYYCVRHGNFGNSYVSWFAYWGQGTLVTVSSGGGGSGGGGSGGGGSQAVVTQEPSLTVSPGGTVTLTCGSSTGAVTTSNYANWVQQKPGQAPRGLIGGTNKRAPGVPARFSGSLLGGKAALTLSGAQPEDEAEYYCALWYSNLWVFGGGTKLTVLGSHHHHHH

SEQ ID NO: 128 - protein, 5 aa, synthetic construct
  REGION 1..5: note = Description of Artificial Sequence: Synthetic peptide
RYTMH

SEQ ID NO: 129 - protein, 10 aa, synthetic construct
  REGION 1..10: note = Description of Artificial Sequence: Synthetic peptide
SASSSVSYMN

SEQ ID NO: 130 - protein, 5 aa, synthetic construct
  REGION 1..5: note = Description of Artificial Sequence: Synthetic peptide
TYAMN

SEQ ID NO: 131 - protein, 14 aa, synthetic construct
  REGION 1..14: note = Description of Artificial Sequence: Synthetic peptide
RSSTGAVTTSNYAN

SEQ ID NO: 132 - protein, 5 aa, synthetic construct
  REGION 1..5: note = Description of Artificial Sequence: Synthetic peptide
SYTMH

SEQ ID NO: 133 - protein, 10 aa, synthetic construct
  REGION 1..10: note = Description of Artificial Sequence: Synthetic peptide
RASSSVSYMH

SEQ ID NO: 134 - protein, 10 aa, synthetic construct
  REGION 1..10: note = Description of Artificial Sequence: Synthetic peptide
RASSSVSYMN

SEQ ID NO: 135 - protein, 5 aa, synthetic construct
  REGION 1..5: note = Description of Artificial Sequence: Synthetic peptide
GYGMH

SEQ ID NO: 136 - protein, 11 aa, synthetic construct
  REGION 1..11: note = Description of Artificial Sequence: Synthetic peptide
RASQSVSSYLA

SEQ ID NO: 137 - protein, 5 aa, synthetic construct
  REGION 1..5: note = Description of Artificial Sequence: Synthetic peptide
SYAMN

SEQ ID NO: 138 - protein, 14 aa, synthetic construct
  REGION 1..14: note = Description of Artificial Sequence: Synthetic peptide
GSSTGAVTSGNYPN

SEQ ID NO: 139 - protein, 5 aa, synthetic construct
  REGION 1..5: note = Description of Artificial Sequence: Synthetic peptide
KYAMN

SEQ ID NO: 140 - protein, 14 aa, synthetic construct
  REGION 1..14: note = Description of Artificial Sequence: Synthetic peptide
GSSTGAVTTSNYAN

SEQ ID NO: 141 - protein, 5 aa, synthetic construct
  REGION 1..5: note = Description of Artificial Sequence: Synthetic peptide
GYTMN

SEQ ID NO: 142 - protein, 11 aa, synthetic construct
  REGION 1..11: note = Description of Artificial Sequence: Synthetic peptide
RASQDIRNYLN

SEQ ID NO: 143 - protein, 5 aa, synthetic construct
  REGION 1..5: note = Description of Artificial Sequence: Synthetic peptide
TYAMS

SEQ ID NO: 144 - protein, 5 aa, synthetic construct
  REGION 1..5: note = Description of Artificial Sequence: Synthetic peptide
TYAMH

SEQ ID NO: 145 - protein, 14 aa, synthetic construct
  REGION 1..14: note = Description of Artificial Sequence: Synthetic peptide
GSSTGAVTSSNYAN

SEQ ID NO: 146 - protein, 14 aa, synthetic construct
  REGION 1..14: note = Description of Artificial Sequence: Synthetic peptide
GSSTGAVTSGHYAN

SEQ ID NO: 147 - protein, 14 aa, synthetic construct
  REGION 1..14: note = Description of Artificial Sequence: Synthetic peptide
KSSTGAVTTSNYAN

SEQ ID NO: 148 - protein, 17 aa, synthetic construct
  REGION 1..17: note = Description of Artificial Sequence: Synthetic peptide
YINPSRGYTNYNQKFKD

SEQ ID NO: 149 - protein, 7 aa, synthetic construct
  REGION 1..7: note = Description of Artificial Sequence: Synthetic peptide
DTSKLAS

SEQ ID NO: 150 - protein, 19 aa, synthetic construct
  REGION 1..19: note = Description of Artificial Sequence: Synthetic peptide
RIRSKYNNYATYYADSVKD

SEQ ID NO: 151 - protein, 7 aa, synthetic construct
  REGION 1..7: note = Description of Artificial Sequence: Synthetic peptide
GTNKRAP

SEQ ID NO: 152 - protein, 17 aa, synthetic construct
  REGION 1..17: note = Description of Artificial Sequence: Synthetic peptide
YINPSSGYTKYNQKFKD

SEQ ID NO: 153 - protein, 7 aa, synthetic construct
  REGION 1..7: note = Description of Artificial Sequence: Synthetic peptide
ATSNLAS

SEQ ID NO: 154 - protein, 7 aa, synthetic construct
  REGION 1..7: note = Description of Artificial Sequence: Synthetic peptide
DTSKVAS

SEQ ID NO: 155 - protein, 17 aa, synthetic construct
  REGION 1..17: note = Description of Artificial Sequence: Synthetic peptide
YINPSRGYTNYNQKVKD

SEQ ID NO: 156 - protein, 17 aa, synthetic construct
  REGION 1..17: note = Description of Artificial Sequence: Synthetic peptide
VIWYDGSKKYYVDSVKG

SEQ ID NO: 157 - protein, 7 aa, synthetic construct
  REGION 1..7: note = Description of Artificial Sequence: Synthetic peptide
DASNRAT

SEQ ID NO: 158 - protein, 15 aa, synthetic construct
  REGION 1..15: note = Description of Artificial Sequence: Synthetic peptide
RIRSKYNNYATYYAD

SEQ ID NO: 159 - protein, 19 aa, synthetic construct
  REGION 1..19: note = Description of Artificial Sequence: Synthetic peptide
RIRSKYNNYATYYADSVKG

SEQ ID NO: 160 - protein, 7 aa, synthetic construct
  REGION 1..7: note = Description of Artificial Sequence: Synthetic peptide
GTKFLAP

SEQ ID NO: 161 - protein, 17 aa, synthetic construct
  REGION 1..17: note = Description of Artificial Sequence: Synthetic peptide
LINPYKGVSTYNQKFKD

SEQ ID NO: 162 - protein, 6 aa, synthetic construct
  REGION 1..6: note = Description of Artificial Sequence: Synthetic peptide
YTSRLH

SEQ ID NO: 163 - protein, 19 aa, synthetic construct
  REGION 1..19: note = Description of Artificial Sequence: Synthetic peptide
RIRSKANNYATYYADSVKG

SEQ ID NO: 164 - protein, 20 aa, synthetic construct
  REGION 1..20: note = Description of Artificial Sequence: Synthetic peptide
RIRSKANNYYATYYADSVKG

SEQ ID NO: 165 - protein, 19 aa, synthetic construct
  REGION 1..19: note = Description of Artificial Sequence: Synthetic peptide
RIRSKANSYATYYADSVKG

SEQ ID NO: 166 - protein, 19 aa, synthetic construct
  REGION 1..19: note = Description of Artificial Sequence: Synthetic peptide
RIRSKYNNYATAYADSVKG

SEQ ID NO: 167 - protein, 7 aa, synthetic construct
  REGION 1..7: note = Description of Artificial Sequence: Synthetic peptide
DTNKRAP

SEQ ID NO: 168 - protein, 7 aa, synthetic construct
  REGION 1..7: note = Description of Artificial Sequence: Synthetic peptide
GTNNRAP

SEQ ID NO: 169 - protein, 7 aa, synthetic construct
  REGION 1..7: note = Description of Artificial Sequence: Synthetic peptide
GTNKRAS

SEQ ID NO: 170 - protein, 7 aa, synthetic construct
  REGION 1..7: note = Description of Artificial Sequence: Synthetic peptide
GTSNKHS

SEQ ID NO: 171 - protein, 18 aa, synthetic construct
  REGION 1..18: note = Description of Artificial Sequence: Synthetic peptide
RIRSNGGYSTYYADSVKG

SEQ ID NO: 172 - protein, 10 aa, synthetic construct
  REGION 1..10: note = Description of Artificial Sequence: Synthetic peptide
YYDDHYCLDY

SEQ ID NO: 173 - protein, 9 aa, synthetic construct
  REGION 1..9: note = Description of Artificial Sequence: Synthetic peptide
QQWSSNPFT

SEQ ID NO: 174 - protein, 14 aa, synthetic construct
  REGION 1..14: note = Description of Artificial Sequence: Synthetic peptide
HGNFGNSYVSWFAY

SEQ ID NO: 175 - protein, 9 aa, synthetic construct
  REGION 1..9: note = Description of Artificial Sequence: Synthetic peptide
ALWYSNLWV

SEQ ID NO: 176 - protein, 10 aa, synthetic construct
  REGION 1..10: note = Description of Artificial Sequence: Synthetic peptide
WQDYDVYFDY

SEQ ID NO: 177 - protein, 9 aa, synthetic construct
  REGION 1..9: note = Description of Artificial Sequence: Synthetic peptide
QQWSSNPPT

SEQ ID NO: 178 - protein, 9 aa, synthetic construct
  REGION 1..9: note = Description of Artificial Sequence: Synthetic peptide
QQWSSNPLT

SEQ ID NO: 179 - protein, 9 aa, synthetic construct
  REGION 1..9: note = Description of Artificial Sequence: Synthetic peptide
QMGYWHFDL

SEQ ID NO: 180 - protein, 10 aa, synthetic construct
  REGION 1..10: note = Description of Artificial Sequence: Synthetic peptide
QQRSNWPPLT

SEQ ID NO: 181 - protein, 16 aa, synthetic construct
  REGION 1..16: note = Description of Artificial Sequence: Synthetic peptide
VRHGNFGNSYVSWFAY

SEQ ID NO: 182 - protein, 14 aa, synthetic construct
  REGION 1..14: note = Description of Artificial Sequence: Synthetic peptide
HGNFGNSYVSWWAY

SEQ ID NO: 183 - protein, 9 aa, synthetic construct
  REGION 1..9: note = Description of Artificial Sequence: Synthetic peptide
VLWYSNRWV

SEQ ID NO: 184 - protein, 14 aa, synthetic construct
  REGION 1..14: note = Description of Artificial Sequence: Synthetic peptide
HGNFGNSYISYWAY

SEQ ID NO: 185 - protein, 14 aa, synthetic construct
  REGION 1..14: note = Description of Artificial Sequence: Synthetic peptide
HGNFGDSYVSWFAY

SEQ ID NO: 186 - protein, 9 aa, synthetic construct
  REGION 1..9: note = Description of Artificial Sequence: Synthetic peptide
ALWYSNHWV

SEQ ID NO: 187 - protein, 10 aa, synthetic construct
  REGION 1..10: note = Description of Artificial Sequence: Synthetic peptide
YYDDHYSLDY

SEQ ID NO: 188 - protein, 13 aa, synthetic construct
  REGION 1..13: note = Description of Artificial Sequence: Synthetic peptide
SGYYGDSDWYFDV

SEQ ID NO: 189 - protein, 9 aa, synthetic construct
  REGION 1..9: note = Description of Artificial Sequence: Synthetic peptide
QQGNTLPWT

SEQ ID NO: 190 - protein, 14 aa, synthetic construct
  REGION 1..14: note = Description of Artificial Sequence: Synthetic peptide
HGNFGDEYVSWFAY

SEQ ID NO: 191 - protein, 14 aa, synthetic construct
  REGION 1..14: note = Description of Artificial Sequence: Synthetic peptide
HGNFGDPYVSWFAY

SEQ ID NO: 192 - protein, 14 aa, synthetic construct
  REGION 1..14: note = Description of Artificial Sequence: Synthetic peptide
HGNFGDSYVSWFDY

SEQ ID NO: 193 - protein, 14 aa, synthetic construct
  REGION 1..14: note = Description of Artificial Sequence: Synthetic peptide
HGNFGQSYVSWFAY

SEQ ID NO: 194 - protein, 14 aa, synthetic construct
  REGION 1..14: note = Description of Artificial Sequence: Synthetic peptide
HGNFGNSYVSWFDY

SEQ ID NO: 195 - protein, 9 aa, synthetic construct
  REGION 1..9: note = Description of Artificial Sequence: Synthetic peptide
LLWYSNLWV

SEQ ID NO: 196 - protein, 7 aa, synthetic construct
  REGION 1..7: note = Description of Artificial Sequence: Synthetic peptide
GYTFTRY

SEQ ID NO: 197 - protein, 6 aa, synthetic construct
  REGION 1..6: note = Description of Artificial Sequence: Synthetic peptide
SSSVSY

SEQ ID NO: 198 - protein, 7 aa, synthetic construct
  REGION 1..7: note = Description of Artificial Sequence: Synthetic peptide
GFTFNTY

SEQ ID NO: 199 - protein, 10 aa, synthetic construct
  REGION 1..10: note = Description of Artificial Sequence: Synthetic peptide
STGAVTTSNY

SEQ ID NO: 200 - protein, 7 aa, synthetic construct
  REGION 1..7: note = Description of Artificial Sequence: Synthetic peptide
GYTFTSY

SEQ ID NO: 201 - protein, 7 aa, synthetic construct
  REGION 1..7: note = Description of Artificial Sequence: Synthetic peptide
GFKFSGY

SEQ ID NO: 202 - protein, 7 aa, synthetic construct
  REGION 1..7: note = Description of Artificial Sequence: Synthetic peptide
SQSVSSY

SEQ ID NO: 203 - protein, 7 aa, synthetic construct
  REGION 1..7: note = Description of Artificial Sequence: Synthetic peptide
GFTFSTY

SEQ ID NO: 204 - protein, 7 aa, synthetic construct
  REGION 1..7: note = Description of Artificial Sequence: Synthetic peptide
GFTFNSY

SEQ ID NO: 205 - protein, 10 aa, synthetic construct
  REGION 1..10: note = Description of Artificial Sequence: Synthetic peptide
STGAVTSGNY

SEQ ID NO: 206 - protein, 7 aa, synthetic construct
  REGION 1..7: note = Description of Artificial Sequence: Synthetic peptide
GFTFNKY

SEQ ID NO: 207 - protein, 7 aa, synthetic construct
  REGION 1..7: note = Description of Artificial Sequence: Synthetic peptide
GYSFTGY

SEQ ID NO: 208 - protein, 7 aa, synthetic construct
  REGION 1..7: note = Description of Artificial Sequence: Synthetic peptide
SQDIRNY

SEQ ID NO: 209 - protein, 6 aa, synthetic construct
  REGION 1..6: note = Description of Artificial Sequence: Synthetic peptide
NPSRGY

SEQ ID NO: 210 - skipped

SEQ ID NO: 211 - protein, 8 aa, synthetic construct
  REGION 1..8: note = Description of Artificial Sequence: Synthetic peptide
RSKYNNYA

SEQ ID NO: 212 - skipped

SEQ ID NO: 213 - protein, 6 aa, synthetic construct
  REGION 1..6: note = Description of Artificial Sequence: Synthetic peptide
NPSSGY

SEQ ID NO: 214 - skipped

SEQ ID NO: 215 - protein, 6 aa, synthetic construct
  REGION 1..6: note = Description of Artificial Sequence: Synthetic peptide
WYDGSK

SEQ ID NO: 216 - skipped

SEQ ID NO: 217 - protein, 9 aa, synthetic construct
  REGION 1..9: note = Description of Artificial Sequence: Synthetic peptide
RSKYNNYAT

SEQ ID NO: 218 - skipped

SEQ ID NO: 219 - protein, 6 aa, synthetic construct
  REGION 1..6: note = Description of Artificial Sequence: Synthetic peptide
NPYKGV

SEQ ID NO: 220 - skipped

SEQ ID NO: 221 - protein, 6 aa, synthetic construct
  REGION 1..6: note = Description of Artificial Sequence: Synthetic peptide
WSSNPF

SEQ ID NO: 222 - protein, 6 aa, synthetic construct
  REGION 1..6: note = Description of Artificial Sequence: Synthetic peptide
WYSNLW

SEQ ID NO: 223 - protein, 6 aa, synthetic construct
  REGION 1..6: note = Description of Artificial Sequence: Synthetic peptide
WSSNPP

SEQ ID NO: 224 - protein, 6 aa, synthetic construct
  REGION 1..6: note = Description of Artificial Sequence: Synthetic peptide
WSSNPL

SEQ ID NO: 225 - protein, 7 aa, synthetic construct
  REGION 1..7: note = Description of Artificial Sequence: Synthetic peptide
RSNWPPL

SEQ ID NO: 226 - protein, 13 aa, synthetic construct
  REGION 1..13: note = Description of Artificial Sequence: Synthetic peptide
HGNFGNSYVSWFA

SEQ ID NO: 227 - protein, 6 aa, synthetic construct
  REGION 1..6: note = Description of Artificial Sequence: Synthetic peptide
WYSNRW

SEQ ID NO: 228 - protein, 6 aa, synthetic construct
  REGION 1..6: note = Description of Artificial Sequence: Synthetic peptide
WYSNHW

SEQ ID NO: 229 - protein, 6 aa, synthetic construct
  REGION 1..6: note = Description of Artificial Sequence: Synthetic peptide
GNTLPW

SEQ ID NO: 230 - protein, 10 aa, synthetic construct
  REGION 1..10: note = Description of Artificial Sequence: Synthetic peptide
GYTFTRYTMH

SEQ ID NO: 231 - protein, 10 aa, synthetic construct
  REGION 1..10: note = Description of Artificial Sequence: Synthetic peptide
GFTFNTYAMN

SEQ ID NO: 232 - protein, 10 aa, synthetic construct
  REGION 1..10: note = Description of Artificial Sequence: Synthetic peptide
GYTFTSYTMH

SEQ ID NO: 233 - protein, 10 aa, synthetic construct
  REGION 1..10: note = Description of Artificial Sequence: Synthetic peptide
GFKFSGYGMH

SEQ ID NO: 234 - protein, 10 aa, synthetic construct
  REGION 1..10: note = Description of Artificial Sequence: Synthetic peptide
GFTFSTYAMN

SEQ ID NO: 235 - protein, 10 aa, synthetic construct
  REGION 1..10: note = Description of Artificial Sequence: Synthetic peptide
GFTFNSYAMN

SEQ ID NO: 236 - protein, 10 aa, synthetic construct
  REGION 1..10: note = Description of Artificial Sequence: Synthetic peptide
GFTFNKYAMN

SEQ ID NO: 237 - protein, 10 aa, synthetic construct
  REGION 1..10: note = Description of Artificial Sequence: Synthetic peptide
GYSFTGYTMN

SEQ ID NO: 238 - protein, 18 aa, synthetic construct
  REGION 1..18: note = Description of Artificial Sequence: Synthetic peptide
RIRSKYNNYATYYADSVK

SEQ ID NO: 239 - protein, 7 aa, synthetic construct
  REGION 1..7: note = Description of Artificial Sequence: Synthetic peptide
YTSRLHS

SEQ ID NO: 240 - protein, 13 aa, synthetic construct
  REGION 1..13: note = Description of Artificial Sequence: Synthetic peptide
KASGYKFTSYVMH

SEQ ID NO: 241 - protein, 16 aa, synthetic construct
  REGION 1..16: note = Description of Artificial Sequence: Synthetic peptide
YINPYNDVTKYNEKFK

SEQ ID NO: 242 - protein, 11 aa, synthetic construct
  REGION 1..11: note = Description of Artificial Sequence: Synthetic peptide
GSYYDYDGFVY

SEQ ID NO: 243 - protein, 10 aa, synthetic construct
  REGION 1..10: note = Description of Artificial Sequence: Synthetic peptide
SATSSVSYMH

SEQ ID NO: 244 - protein, 120 aa, synthetic construct
  REGION 1..120: note = Description of Artificial Sequence: Synthetic polypeptide
EVQLQQSGPELVKPGASVKMSCKASGYKFTSYVMHWVKQKPGQGLEWIGYINPYNDVTKYNEKFKGKATLTSDKSSSTAYMELSSLTSEDSAVHYCARGSYYDYDGFVYWGQGTLVTVSA

SEQ ID NO: 245 - protein, 106 aa, synthetic construct
  REGION 1..106: note = Description of Artificial Sequence: Synthetic polypeptide
QIVLTQSPAIMSASPGEKVTMTCSATSSVSYMHWYQQKSGTSPKRWIYDTSKLASGVPARFSGSGSGTSYSLTISSMEAEDAATYYCQQWSSNPLTFGAGTKLELK

SEQ ID NO: 246 - protein, 115 aa, synthetic construct
  REGION 1..115: note = Description of Artificial Sequence: Synthetic polypeptide
EVQLVESGGGLVQPGGSLRLSCAVSGFALSNHGMSWVRRAPGKGLEWVSGIVYSGSTYYAASVKGRFTISRDNSRNTLYLQMNSLRPEDTAIYYCSAHGGESDVWGQGTTVTVSS

SEQ ID NO: 247 - protein, 107 aa, synthetic construct
  REGION 1..107: note = Description of Artificial Sequence: Synthetic polypeptide
DIQLTQSPSSLSASVGDRVTITCRASQSISSYLNWYQQKPGKAPKLLIYAASSLQSGVPSRFSGSGSGTDFTLTISSLQPEDFATYYCQQSYSTPYTFGQGTKVEIK

SEQ ID NO: 248 - protein, 239 aa, synthetic construct
  REGION 1..239: note = Description of Artificial Sequence: Synthetic polypeptide
EVQLVESGGGLVQPGGSLRLSCAVSGFALSNHGMSWVRRAPGKGLEWVSGIVYSGSTYYAASVKGRFTISRDNSRNTLYLQMNSLRPEDTAIYYCSAHGGESDVWGQGTTVTVSSASGGGGSGGRASGGGGSDIQLTQSPSSLSASVGDRVTITCRASQSISSYLNWYQQKPGKAPKLLIYAASSLQSGVPSRFSGSGSGTDFTLTISSLQPEDFATYYCQQSYSTPYTFGQGTKVEIK

SEQ ID NO: 249 - protein, 120 aa, synthetic construct
  REGION 1..120: note = Description of Artificial Sequence: Synthetic polypeptide
QVQLVESGGGLVQPGRSLRLSCAASGFTFSNYAMSWVRQAPGKGLGWVSGISRSGENTYYADSVKGRFTISRDNSKNTLYLQMNSLRDEDTAVYYCARSPAHYYGGMDVWGQGTTVTVSS

SEQ ID NO: 250 - protein, 109 aa, synthetic construct
  REGION 1..109: note = Description of Artificial Sequence: Synthetic polypeptide
DIVLTQSPGTLSLSPGERATLSCRASQSISSSFLAWYQQKPGQAPRLLIYGASRRATGIPDRFSGSGSGTDFTLTISRLEPEDSAVYYCQQYHSSPSWTFGQGTKLEIK

SEQ ID NO: 251 - protein, 246 aa, synthetic construct
  REGION 1..246: note = Description of Artificial Sequence: Synthetic polypeptide
QVQLVESGGGLVQPGRSLRLSCAASGFTFSNYAMSWVRQAPGKGLGWVSGISRSGENTYYADSVKGRFTISRDNSKNTLYLQMNSLRDEDTAVYYCARSPAHYYGGMDVWGQGTTVTVSSASGGGGSGGRASGGGGSDIVLTQSPGTLSLSPGERATLSCRASQSISSSFLAWYQQKPGQAPRLLIYGASRRATGIPDRFSGSGSGTDFTLTISRLEPEDSAVYYCQQYHSSPSWTFGQGTKLEIK

SEQ ID NO: 252 - protein, 115 aa, synthetic construct
  REGION 1..115: note = Description of Artificial Sequence: Synthetic polypeptide
QVQLVESGGGLVQPGRSLRLSCAASGFTFDDYAMHWVRQAPGKGLEWVSGISWNSGSIGYADSVKGRFTISRDNAKNSLYLQMNSLRAEDTALYYCSVHSFLAYWGQGTLVTVSS

SEQ ID NO: 253 - protein, 112 aa, synthetic construct
  REGION 1..112: note = Description of Artificial Sequence: Synthetic polypeptide
DIVMTQTPLSLPVTPGEPASISCRSSQSLLHSNGYNYLDWYLQKPGQSPQLLIYLGSNRASGVPDRFSGSGSGTDFTLKISRVEAEDVGVYYCMQALQTPYTFGQGTKVEIK

SEQ ID NO: 254 - protein, 244 aa, synthetic construct
  REGION 1..244: note = Description of Artificial Sequence: Synthetic polypeptide
QVQLVESGGGLVQPGRSLRLSCAASGFTFDDYAMHWVRQAPGKGLEWVSGISWNSGSIGYADSVKGRFTISRDNAKNSLYLQMNSLRAEDTALYYCSVHSFLAYWGQGTLVTVSSASGGGGSGGRASGGGGSDIVMTQTPLSLPVTPGEPASISCRSSQSLLHSNGYNYLDWYLQKPGQSPQLLIYLGSNRASGVPDRFSGSGSGTDFTLKISRVEAEDVGVYYCMQALQTPYTFGQGTKVEIK

SEQ ID NO: 255 - protein, 115 aa, synthetic construct
  REGION 1..115: note = Description of Artificial Sequence: Synthetic polypeptide
EVQLLESGGGLVQPGGSLRLSCAVSGFALSNHGMSWVRRAPGKGLEWVSGIVYSGSTYYAASVKGRFTISRDNSRNTLYLQMNSLRPEDTAIYYCSAHGGESDVWGQGTTVTVSS

SEQ ID NO: 256 - protein, 111 aa, synthetic construct
  REGION 1..111: note = Description of Artificial Sequence: Synthetic polypeptide
DIVMTQTPLSLSVTPGQPASISCKSSQSLLRNDGKTPLYWYLQKAGQPPQLLIYEVSNRFSGVPDRFSGSGSGTDFTLKISRVEAEDVGAYYCMQNIQFPSFGGGTKLEIK

SEQ ID NO: 257 - protein, 243 aa, synthetic construct
  REGION 1..243: note = Description of Artificial Sequence: Synthetic polypeptide
EVQLLESGGGLVQPGGSLRLSCAVSGFALSNHGMSWVRRAPGKGLEWVSGIVYSGSTYYAASVKGRFTISRDNSRNTLYLQMNSLRPEDTAIYYCSAHGGESDVWGQGTTVTVSSASGGGGSGGRASGGGGSDIVMTQTPLSLSVTPGQPASISCKSSQSLLRNDGKTPLYWYLQKAGQPPQLLIYEVSNRFSGVPDRFSGSGSGTDFTLKISRVEAEDVGAYYCMQNIQFPSFGGGTKLEIK

SEQ ID NO: 258 - protein, 120 aa, synthetic construct
  REGION 1..120: note = Description of Artificial Sequence: Synthetic polypeptide
QVQLVQSGAEVRKTGASVKVSCKASGYIFDNFGINWVRQAPGQGLEWMGWINPKNNNTNYAQKFQGRVTITADESTNTAYMEVSSLRSEDTAVYYCARGPYYYQSYMDVWGQGTMVTVSS

SEQ ID NO: 259 - protein, 112 aa, synthetic construct
  REGION 1..112: note = Description of Artificial Sequence: Synthetic polypeptide
DIVMTQTPLSLPVTPGEPASISCRSSQSLLHSNGYNYLNWYLQKPGQSPQLLIYLGSKRASGVPDRFSGSGSGTDFTLHITRVGAEDVGVYYCMQALQTPYTFGQGTKLEIK

SEQ ID NO: 260 - protein, 249 aa, synthetic construct
  REGION 1..249: note = Description of Artificial Sequence: Synthetic polypeptide
QVQLVQSGAEVRKTGASVKVSCKASGYIFDNFGINWVRQAPGQGLEWMGWINPKNNNTNYAQKFQGRVTITADESTNTAYMEVSSLRSEDTAVYYCARGPYYYQSYMDVWGQGTMVTVSSASGGGGSGGRASGGGGSDIVMTQTPLSLPVTPGEPASISCRSSQSLLHSNGYNYLNWYLQKPGQSPQLLIYLGSKRASGVPDRFSGSGSGTDFTLHITRVGAEDVGVYYCMQALQTPYTFGQGTKLEIK

SEQ ID NO: 261 - protein, 123 aa, synthetic construct
  REGION 1..123: note = Description of Artificial Sequence: Synthetic polypeptide
QVQLQESGGGLVQPGGSLRLSCAASGFTFSSDAMTWVRQAPGKGLEWVSVISGSGGTTYYADSVKGRFTISRDNSKNTLYLQMNSLRAEDTAVYYCAKLDSSGYYYARGPRYWGQGTLVTVSS

SEQ ID NO: 262 - protein, 106 aa, synthetic construct
  REGION 1..106: note = Description of Artificial Sequence: Synthetic polypeptide
DIQLTQSPSSLSASVGDRVTITCRASQSISSYLNWYQQKPGKAPKLLIYGASTLASGVPARFSGSGSGTHFTLTINSLQSEDSATYYCQQSYKRASFGQGTKVEIK

SEQ ID NO: 263 - protein, 246 aa, synthetic construct
  REGION 1..246: note = Description of Artificial Sequence: Synthetic polypeptide
QVQLQESGGGLVQPGGSLRLSCAASGFTFSSDAMTWVRQAPGKGLEWVSVISGSGGTTYYADSVKGRFTISRDNSKNTLYLQMNSLRAEDTAVYYCAKLDSSGYYYARGPRYWGQGTLVTVSSASGGGGSGGRASGGGGSDIQLTQSPSSLSASVGDRVTITCRASQSISSYLNWYQQKPGKAPKLLIYGASTLASGVPARFSGSGSGTHFTLTINSLQSEDSATYYCQQSYKRASFGQGTKVEIK

SEQ ID NO: 264 - protein, 118 aa, synthetic construct
  REGION 1..118: note = Description of Artificial Sequence: Synthetic polypeptide
QVQLVQSGAEVKKPGASVKVSCKASGYTFSNYGITWVRQAPGQGLEWMGWISAYNGNTNYAQKFQGRVTMTRNTSISTAYMELSSLRSEDTAVYYCARGPYYYYMDVWGKGTMVTVSS

SEQ ID NO: 265 - protein, 112 aa, synthetic construct
  REGION 1..112: note = Description of Artificial Sequence: Synthetic polypeptide
EIVMTQSPLSLPVTPGEPASISCRSSQSLLYSNGYNYVDWYLQKPGQSPQLLIYLGSNRASGVPDRFSGSGSGTDFKLQISRVEAEDVGIYYCMQGRQFPYSFGQGTKVEIK

SEQ ID NO: 266 - protein, 247 aa, synthetic construct
  REGION 1..247: note = Description of Artificial Sequence: Synthetic polypeptide
QVQLVQSGAEVKKPGASVKVSCKASGYTFSNYGITWVRQAPGQGLEWMGWISAYNGNTNYAQKFQGRVTMTRNTSISTAYMELSSLRSEDTAVYYCARGPYYYYMDVWGKGTMVTVSSASGGGGSGGRASGGGGSEIVMTQSPLSLPVTPGEPASISCRSSQSLLYSNGYNYVDWYLQKPGQSPQLLIYLGSNRASGVPDRFSGSGSGTDFKLQISRVEAEDVGIYYCMQGRQFPYSFGQGTKVEIK

SEQ ID NO: 267 - protein, 115 aa, synthetic construct
  REGION 1..115: note = Description of Artificial Sequence: Synthetic polypeptide
EVQLLETGGGLVQPGGSLRLSCAVSGFALSNHGMSWVRRAPGKGLEWVSGIVYSGSTYYAASVKGRFTISRDNSRNTLYLQMNSLRPEDTAIYYCSAHGGESDVWGQGTTVTVSS

SEQ ID NO: 268 - protein, 106 aa, synthetic construct
  REGION 1..106: note = Description of Artificial Sequence: Synthetic polypeptide
EIVLTQSPATLSVSPGESATLSCRASQSVSSNLAWYQQKPGQAPRLLIYGASTRASGIPDRFSGSGSGTDFTLTISSLQAEDVAVYYCQQYGSSLTFGGGTKVEIK

SEQ ID NO: 269 - protein, 238 aa, synthetic construct
  REGION 1..238: note = Description of Artificial Sequence: Synthetic polypeptide
EVQLLETGGGLVQPGGSLRLSCAVSGFALSNHGMSWVRRAPGKGLEWVSGIVYSGSTYYAASVKGRFTISRDNSRNTLYLQMNSLRPEDTAIYYCSAHGGESDVWGQGTTVTVSSASGGGGSGGRASGGGGSEIVLTQSPATLSVSPGESATLSCRASQSVSSNLAWYQQKPGQAPRLLIYGASTRASGIPDRFSGSGSGTDFTLTISSLQAEDVAVYYCQQYGSSLTFGGGTKVEIK

SEQ ID NO: 270 - protein, 115 aa, synthetic construct
  REGION 1..115: note = Description of Artificial Sequence: Synthetic polypeptide
EVQLVETGGGLVQPGGSLRLSCAVSGFALSNHGMSWVRRAPGKGLEWVSGIVYSGSTYYAASVKGRFTISRDNSRNTLYLQMNSLRPEDTAIYYCSAHGGESDVWGQGTTVTVSS

SEQ ID NO: 271 - protein, 107 aa, synthetic construct
  REGION 1..107: note = Description of Artificial Sequence: Synthetic polypeptide
EIVMTQSPATLSVSPGERATLSCRASQSVSSKLAWYQQKPGQAPRLLMYGASIRATGIPDRFSGSGSGTEFTLTISSLEPEDFAVYYCQQYGSSSWTFGQGTKVEIK

SEQ ID NO: 272 - protein, 239 aa, synthetic construct
  REGION 1..239: note = Description of Artificial Sequence: Synthetic polypeptide
EVQLVETGGGLVQPGGSLRLSCAVSGFALSNHGMSWVRRAPGKGLEWVSGIVYSGSTYYAASVKGRFTISRDNSRNTLYLQMNSLRPEDTAIYYCSAHGGESDVWGQGTTVTVSSASGGGGSGGRASGGGGSEIVMTQSPATLSVSPGERATLSCRASQSVSSKLAWYQQKPGQAPRLLMYGASIRATGIPDRFSGSGSGTEFTLTISSLEPEDFAVYYCQQYGSSSWTFGQGTKVEIK

SEQ ID NO: 273 - protein, 115 aa, synthetic construct
  REGION 1..115: note = Description of Artificial Sequence: Synthetic polypeptide
EVQLVETGGGVVQPGGSLRLSCAVSGFALSNHGMSWVRRAPGKGLEWVSGIVYSGSTYYAASVKGRFTISRDNSRNTLYLQMNSLRPEDTAIYYCSAHGGESDVWGQGTTVTVSS

SEQ ID NO: 274 - protein, 109 aa, synthetic construct
  REGION 1..109: note = Description of Artificial Sequence: Synthetic polypeptide
EIVLTQSPGTLSLSPGERATLSCRASQSVGSTNLAWYQQKPGQAPRLLIYDASNRATGIPDRFSGGGSGTDFTLTISRLEPEDFAVYYCQQYGSSPPWTFGQGTKVEIK

SEQ ID NO: 275 - protein, 241 aa, synthetic construct
  REGION 1..241: note = Description of Artificial Sequence: Synthetic polypeptide
EVQLVETGGGVVQPGGSLRLSCAVSGFALSNHGMSWVRRAPGKGLEWVSGIVYSGSTYYAASVKGRFTISRDNSRNTLYLQMNSLRPEDTAIYYCSAHGGESDVWGQGTTVTVSSASGGGGSGGRASGGGGSEIVLTQSPGTLSLSPGERATLSCRASQSVGSTNLAWYQQKPGQAPRLLIYDASNRATGIPDRFSGGGSGTDFTLTISRLEPEDFAVYYCQQYGSSPPWTFGQGTKVEIK

SEQ ID NO: 276 - protein, 117 aa, synthetic construct
  REGION 1..117: note = Description of Artificial Sequence: Synthetic polypeptide
QVQLVESGGGLVKPGGSLRLSCAASGFTFSDYYMSWIRQAPGKGLEWVSYISSSGSTIYYADSVKGRFTISRDNAKNSLYLQMNSLRAEDTAVYYCARESGDGMDVWGQGTTVTVSS

SEQ ID NO: 277 - protein, 105 aa, synthetic construct
  REGION 1..105: note = Description of Artificial Sequence: Synthetic polypeptide
DIQMTQSPSSLSASVGDRVTITCRASQSISSYLNWYQQKPGKAPKLLIYAASSLQSGVPSRFSGSGSGTDFTLTISSLQPEDFATYYCQQSYTLAFGQGTKVDIK

SEQ ID NO: 278 - protein, 239 aa, synthetic construct
  REGION 1..239: note = Description of Artificial Sequence: Synthetic polypeptide
QVQLVESGGGLVKPGGSLRLSCAASGFTFSDYYMSWIRQAPGKGLEWVSYISSSGSTIYYADSVKGRFTISRDNAKNSLYLQMNSLRAEDTAVYYCARESGDGMDVWGQGTTVTVSSASGGGGSGGRASGGGGSDIQMTQSPSSLSASVGDRVTITCRASQSISSYLNWYQQKPGKAPKLLIYAASSLQSGVPSRFSGSGSGTDFTLTISSLQPEDFATYYCQQSYTLAFGQGTKVDIK

SEQ ID NO: 279 - protein, 117 aa, synthetic construct
  REGION 1..117: note = Description of Artificial Sequence: Synthetic polypeptide
QVQLVQSGGGLVKPGGSLRLSCAASGFTFSDYYMSWIRQAPGKGLEWVSYISSSGNTIYYADSVKGRFTISRDNAKNSLYLQMNSLRAEDTAVYYCARSTMVREDYWGQGTLVTVSS

SEQ ID NO: 280 - protein, 112 aa, synthetic construct
  REGION 1..112: note = Description of Artificial Sequence: Synthetic polypeptide
DIVLTQSPLSLPVTLGQPASISCKSSESLVHNSGKTYLNWFHQRPGQSPRRLIYEVSNRDSGVPDRFTGSGSGTDFTLKISRVEAEDVGVYYCMQGTHWPGTFGQGTKLEIK

SEQ ID NO: 281 - protein, 246 aa, synthetic construct
  REGION 1..246: note = Description of Artificial Sequence: Synthetic polypeptide
QVQLVQSGGGLVKPGGSLRLSCAASGFTFSDYYMSWIRQAPGKGLEWVSYISSSGNTIYYADSVKGRFTISRDNAKNSLYLQMNSLRAEDTAVYYCARSTMVREDYWGQGTLVTVSSASGGGGSGGRASGGGGSDIVLTQSPLSLPVTLGQPASISCKSSESLVHNSGKTYLNWFHQRPGQSPRRLIYEVSNRDSGVPDRFTGSGSGTDFTLKISRVEAEDVGVYYCMQGTHWPGTFGQGTKLEIK

SEQ ID NO: 282 - protein, 115 aa, synthetic construct
  REGION 1..115: note = Description of Artificial Sequence: Synthetic polypeptide
QVQLVESGGGLVQPGGSLRLSCAVSGFALSNHGMSWVRRAPGKGLEWVSGIVYSGSTYYAASVKGRFTISRDNSRNTLYLQMNSLRPEDTAIYYCSAHGGESDVWGQGTTVTVSS

SEQ ID NO: 283 - protein, 107 aa, synthetic construct
  REGION 1..107: note = Description of Artificial Sequence: Synthetic polypeptide
DIRLTQSPSPLSASVGDRVTITCQASEDINKFLNWYHQTPGKAPKLLIYDASTLQTGVPSRFSGSGSGTDFTLTINSLQPEDIGTYYCQQYESLPLTFGGGTKVEIK

SEQ ID NO: 284 - protein, 239 aa, synthetic construct
  REGION 1..239: note = Description of Artificial Sequence: Synthetic polypeptide
QVQLVESGGGLVQPGGSLRLSCAVSGFALSNHGMSWVRRAPGKGLEWVSGIVYSGSTYYAASVKGRFTISRDNSRNTLYLQMNSLRPEDTAIYYCSAHGGESDVWGQGTTVTVSSASGGGGSGGRASGGGGSDIRLTQSPSPLSASVGDRVTITCQASEDINKFLNWYHQTPGKAPKLLIYDASTLQTGVPSRFSGSGSGTDFTLTINSLQPEDIGTYYCQQYESLPLTFGGGTKVEIK

SEQ ID NO: 285 - protein, 108 aa, synthetic construct
  REGION 1..108: note = Description of Artificial Sequence: Synthetic polypeptide
ETTLTQSPATLSVSPGERATLSCRASQSVGSNLAWYQQKPGQGPRLLIYGASTRATGIPARFSGSGSGTEFTLTISSLQPEDFAVYYCQQYNDWLPVTFGQGTKVEIK

SEQ ID NO: 286 - protein, 240 aa, synthetic construct
  REGION 1..240: note = Description of Artificial Sequence: Synthetic polypeptide
EVQLVETGGGLVQPGGSLRLSCAVSGFALSNHGMSWVRRAPGKGLEWVSGIVYSGSTYYAASVKGRFTISRDNSRNTLYLQMNSLRPEDTAIYYCSAHGGESDVWGQGTTVTVSSASGGGGSGGRASGGGGSETTLTQSPATLSVSPGERATLSCRASQSVGSNLAWYQQKPGQGPRLLIYGASTRATGIPARFSGSGSGTEFTLTISSLQPEDFAVYYCQQYNDWLPVTFGQGTKVEIK

SEQ ID NO: 287 - protein, 109 aa, synthetic construct
  REGION 1..109: note = Description of Artificial Sequence: Synthetic polypeptide
EIVLTQSPGTLSLSPGERATLSCRASQSIGSSSLAWYQQKPGQAPRLLMYGASSRASGIPDRFSGSGSGTDFTLTISRLEPEDFAVYYCQQYAGSPPFTFGQGTKVEIK

SEQ ID NO: 288 - protein, 241 aa, synthetic construct
  REGION 1..241: note = Description of Artificial Sequence: Synthetic polypeptide
EVQLVESGGGLVQPGGSLRLSCAVSGFALSNHGMSWVRRAPGKGLEWVSGIVYSGSTYYAASVKGRFTISRDNSRNTLYLQMNSLRPEDTAIYYCSAHGGESDVWGQGTTVTVSSASGGGGSGGRASGGGGSEIVLTQSPGTLSLSPGERATLSCRASQSIGSSSLAWYQQKPGQAPRLLMYGASSRASGIPDRFSGSGSGTDFTLTISRLEPEDFAVYYCQQYAGSPPFTFGQGTKVEIK

SEQ ID NO: 289 - protein, 121 aa, synthetic construct
  REGION 1..121: note = Description of Artificial Sequence: Synthetic polypeptide
QVQLQESGPGLVKPSETLSLTCTVSGGSISSSYYYWGWIRQPPGKGLEWIGSIYYSGSAYYNPSLKSRVTISVDTSKNQFSLRLSSVTAADTAVYYCARHWQEWPDAFDIWGQGTMVTVSS

SEQ ID NO: 290 - protein, 107 aa, synthetic construct
  REGION 1..107: note = Description of Artificial Sequence: Synthetic polypeptide
ETTLTQSPAFMSATPGDKVIISCKASQDIDDAMNWYQQKPGEAPLFIIQSATSPVPGIPPRFSGSGFGTDFSLTINNIESEDAAYYFCLQHDNFPLTFGQGTKLEIK

SEQ ID NO: 291 - protein, 243 aa, synthetic construct
  REGION 1..243: note = Description of Artificial Sequence: Synthetic polypeptide
QVQLQESGPGLVKPSETLSLTCTVSGGSISSSYYYWGWIRQPPGKGLEWIGSIYYSGSAYYNPSLKSRVTISVDTSKNQFSLRLSSVTAADTAVYYCARHWQEWPDAFDIWGQGTMVTVSSGGGGSGGGGSGGGGSETTLTQSPAFMSATPGDKVIISCKASQDIDDAMNWYQQKPGEAPLFIIQSATSPVPGIPPRFSGSGFGTDFSLTINNIESEDAAYYFCLQHDNFPLTFGQGTKLEIK

SEQ ID NO: 292 - protein, 123 aa, synthetic construct
  REGION 1..123: note = Description of Artificial Sequence: Synthetic polypeptide
QVNLRESGPALVKPTQTLTLTCTFSGFSLRTSGMCVSWIRQPPGKALEWLARIDWDEDKFYSTSLKTRLTISKDTSDNQVVLRMTNMDPADTATYYCARSGAGGTSATAFDIWGPGTMVTVSS

SEQ ID NO: 293 - protein, 107 aa, synthetic construct
  REGION 1..107: note = Description of Artificial Sequence: Synthetic polypeptide
DIQMTQSPSSLSASVGDRVTITCRASQDIYNNLAWFQLKPGSAPRSLMYAANKSQSGVPSRFSGSASGTDFTLTISSLQPEDFATYYCQHYYRFPYSFGQGTKLEIK

SEQ ID NO: 294 - protein, 244 aa, synthetic construct
  REGION 1..244: note = Description of Artificial Sequence: Synthetic polypeptide
VNLRESGPALVKPTQTLTLTCTFSGFSLRTSGMCVSWIRQPPGKALEWLARIDWDEDKFYSTSLKTRLTISKDTSDNQVVLRMTNMDPADTATYYCARSGAGGTSATAFDIWGPGTMVTVSSGGGGSGGGGSGGGGSDIQMTQSPSSLSASVGDRVTITCRASQDIYNNLAWFQLKPGSAPRSLMYAANKSQSGVPSRFSGSASGTDFTLTISSLQPEDFATYYCQHYYRFPYSFGQGTKLEIK

SEQ ID NO: 295 - protein, 119 aa, synthetic construct
  REGION 1..119: note = Description of Artificial Sequence: Synthetic polypeptide
EVQLVESGGGLVKPGGSLRLSCAASGFTFSSYSMNWVRQAPGKGLEWVSSISSSSSYIYYADSVKGRFTISRDNAKNSLYLQMNSLRAEDTAVYYCAKTIAAVYAFDIWGQGTTVTVSS

SEQ ID NO: 296 - protein, 112 aa, synthetic construct
  REGION 1..112: note = Description of Artificial Sequence: Synthetic polypeptide
EIVLTQSPLSLPVTPEEPASISCRSSQSLLHSNGYNYLDWYLQKPGQSPQLLIYLGSNRASGVPDRFSGSGSGTDFTLKISRVEAEDVGVYYCMQALQTPYTFGQGTKLEIK

SEQ ID NO: 297 - protein, 246 aa, synthetic construct
  REGION 1..246: note = Description of Artificial Sequence: Synthetic polypeptide
EVQLVESGGGLVKPGGSLRLSCAASGFTFSSYSMNWVRQAPGKGLEWVSSISSSSSYIYYADSVKGRFTISRDNAKNSLYLQMNSLRAEDTAVYYCAKTIAAVYAFDIWGQGTTVTVSSGGGGSGGGGSGGGGSEIVLTQSPLSLPVTPEEPASISCRSSQSLLHSNGYNYLDWYLQKPGQSPQLLIYLGSNRASGVPDRFSGSGSGTDFTLKISRVEAEDVGVYYCMQALQTPYTFGQGTKLEIK

SEQ ID NO: 298 - protein, 117 aa, synthetic construct
  REGION 1..117: note = Description of Artificial Sequence: Synthetic polypeptide
EVQLVESGGGLVKPGGSLRLSCAASGFTFSDYYMSWIRQAPGKGLEWVSYISSSGSTIYYADSVKGRFTISRDNAKNSLYLQMNSLRAEDTAVYYCARDLRGAFDIWGQGTMVTVSS

SEQ ID NO: 299 - protein, 108 aa, synthetic construct
  REGION 1..108: note = Description of Artificial Sequence: Synthetic polypeptide
SYVLTQSPSVSAAPGYTATISCGGNNIGTKSVHWYQQKPGQAPLLVIRDDSVRPSKIPGRFSGSNSGNMATLTISGVQAGDEADFYCQVWDSDSEHVVFGGGTKLTVL

SEQ ID NO: 300 - protein, 240 aa, synthetic construct
  REGION 1..240: note = Description of Artificial Sequence: Synthetic polypeptide
EVQLVESGGGLVKPGGSLRLSCAASGFTFSDYYMSWIRQAPGKGLEWVSYISSSGSTIYYADSVKGRFTISRDNAKNSLYLQMNSLRAEDTAVYYCARDLRGAFDIWGQGTMVTVSSGGGGSGGGGSGGGGSSYVLTQSPSVSAAPGYTATISCGGNNIGTKSVHWYQQKPGQAPLLVIRDDSVRPSKIPGRFSGSNSGNMATLTISGVQAGDEADFYCQVWDSDSEHVVFGGGTKLTVL

SEQ ID NO: 301 - protein, 120 aa, synthetic construct
  REGION 1..120: note = Description of Artificial Sequence: Synthetic polypeptide
QVQLVQSGAEVKKPGASVKVSCKPSGYTVTSHYIHWVRRAPGQGLEWMGMINPSGGVTAYSQTLQGRVTMTSDTSSSTVYMELSSLRSEDTAMYYCAREGSGSGWYFDFWGRGTLVTVSS

SEQ ID NO: 302 - protein, 106 aa, synthetic construct
  REGION 1..106: note = Description of Artificial Sequence: Synthetic polypeptide
SYVLTQPPSVSVSPGQTASITCSGDGLSKKYVSWYQQKAGQSPVVLISRDKERPSGIPDRFSGSNSADTATLTISGTQAMDEADYYCQAWDDTTVVFGGGTKLTVL

SEQ ID NO: 303 - protein, 241 aa, synthetic construct
  REGION 1..241: note = Description of Artificial Sequence: Synthetic polypeptide
QVQLVQSGAEVKKPGASVKVSCKPSGYTVTSHYIHWVRRAPGQGLEWMGMINPSGGVTAYSQTLQGRVTMTSDTSSSTVYMELSSLRSEDTAMYYCAREGSGSGWYFDFWGRGTLVTVSSGGGGSGGGGSGGGGSSYVLTQPPSVSVSPGQTASITCSGDGLSKKYVSWYQQKAGQSPVVLISRDKERPSGIPDRFSGSNSADTATLTISGTQAMDEADYYCQAWDDTTVVFGGGTKLTVL

SEQ ID NO: 304 - protein, 123 aa, synthetic construct
  REGION 1..123: note = Description of Artificial Sequence: Synthetic polypeptide
QVQLQESGPGLVKPSQTLSLTCTVSGGSISSGGYYWSWIRQHPGKGLEWIGYIYYSGSTYYNPSLKSRVTISVDTSKNQFSLKLSSVTAADTAVYYCARAGIAARLRGAFDIWGQGTMVTVSS

SEQ ID NO: 305 - protein, 107 aa, synthetic construct
  REGION 1..107: note = Description of Artificial Sequence: Synthetic polypeptide
DIVMTQSPSSVSASVGDRVIITCRASQGIRNWLAWYQQKPGKAPNLLIYAASNLQSGVPSRFSGSGSGADFTLTISSLQPEDVATYYCQKYNSAPFTFGPGTKVDIK

SEQ ID NO: 306 - protein, 245 aa, synthetic construct
  REGION 1..245: note = Description of Artificial Sequence: Synthetic polypeptide
QVQLQESGPGLVKPSQTLSLTCTVSGGSISSGGYYWSWIRQHPGKGLEWIGYIYYSGSTYYNPSLKSRVTISVDTSKNQFSLKLSSVTAADTAVYYCARAGIAARLRGAFDIWGQGTMVTVSSGGGGSGGGGSGGGGSDIVMTQSPSSVSASVGDRVIITCRASQGIRNWLAWYQQKPGKAPNLLIYAASNLQSGVPSRFSGSGSGADFTLTISSLQPEDVATYYCQKYNSAPFTFGPGTKVDIK

SEQ ID NO: 307 - protein, 129 aa, synthetic construct
  REGION 1..129: note = Description of Artificial Sequence: Synthetic polypeptide
QVQLVQSGAEVKKPGSSVKVSCKASGGTFSSYAISWVRQAPGQGLEWMGGIIPIFGTANYAQKFQGRVTITADESTSTAYMELSSLRSEDTAVYYCARRGGYQLLRWDVGLLRSAFDIWGQGTMVTVSS

SEQ ID NO: 308 - protein, 109 aa, synthetic construct
  REGION 1..109: note = Description of Artificial Sequence: Synthetic polypeptide
SYVLTQPPSVSVAPGQTARITCGGNNIGSKSVHWYQQKPGQAPVLVLYGKNNRPSGVPDRFSGSRSGTTASLTITGAQAEDEADYYCSSRDSSGDHLRVFGTGTKVTVL

SEQ ID NO: 309 - protein, 253 aa, synthetic construct
  REGION 1..253: note = Description of Artificial Sequence: Synthetic polypeptide
QVQLVQSGAEVKKPGSSVKVSCKASGGTFSSYAISWVRQAPGQGLEWMGGIIPIFGTANYAQKFQGRVTITADESTSTAYMELSSLRSEDTAVYYCARRGGYQLLRWDVGLLRSAFDIWGQGTMVTVSSGGGGSGGGGSGGGGSSYVLTQPPSVSVAPGQTARITCGGNNIGSKSVHWYQQKPGQAPVLVLYGKNNRPSGVPDRFSGSRSGTTASLTITGAQAEDEADYYCSSRDSSGDHLRVFGTGTKVTVL

SEQ ID NO: 310 - protein, 125 aa, synthetic construct
  REGION 1..125: note = Description of Artificial Sequence: Synthetic polypeptide
EVQLQQSGPGLVKPSQTLSLTCAISGDSVSSNSAAWNWIRQSPSRGLEWLGRTYYRSKWYSFYAISLKSRIIINPDTSKNQFSLQLKSVTPEDTAVYYCARSSPEGLFLYWFDPWGQGTLVTVSS

SEQ ID NO: 311 - protein, 108 aa, synthetic construct
  REGION 1..108: note = Description of Artificial Sequence: Synthetic polypeptide
SSELTQDPAVSVALGQTIRITCQGDSLGNYYATWYQQKPGQAPVLVIYGTNNRPSGIPDRFSASSSGNTASLTITGAQAEDEADYYCNSRDSSGHHLLFGTGTKVTVL

SEQ ID NO: 312 - protein, 248 aa, synthetic construct
  REGION 1..248: note = Description of Artificial Sequence: Synthetic polypeptide
EVQLQQSGPGLVKPSQTLSLTCAISGDSVSSNSAAWNWIRQSPSRGLEWLGRTYYRSKWYSFYAISLKSRIIINPDTSKNQFSLQLKSVTPEDTAVYYCARSSPEGLFLYWFDPWGQGTLVTVSSGGDGSGGGGSGGGGSSSELTQDPAVSVALGQTIRITCQGDSLGNYYATWYQQKPGQAPVLVIYGTNNRPSGIPDRFSASSSGNTASLTITGAQAEDEADYYCNSRDSSGHHLLFGTGTKVTVL

SEQ ID NO: 313 - protein, 118 aa, synthetic construct
  REGION 1..118: note = Description of Artificial Sequence: Synthetic polypeptide
EVQLVESGGGLVQPGGSLRLSCAASGFTFSSYAMSWVRQAPGKGLEWVSAISGSGGSTYYADSVKGRFTISRDNSKNTLYLQMNSLRAEDTAVYYCAKVEGSGSLDYWGQGTLVTVSS

SEQ ID NO: 314 - protein, 113 aa, synthetic construct
  REGION 1..113: note = Description of Artificial Sequence: Synthetic polypeptide
EIVMTQSPGTLSLSPGERATLSCRASQSVSSAYLAWYQQKPGQPPRLLISGASTRATGIPDRFGGSGSGTDFTLTISRLEPEDFAVYYCQHYGSSFNGSSLFTFGQGTRLEIK

SEQ ID NO: 315 - protein, 246 aa, synthetic construct
  REGION 1..246: note = Description of Artificial Sequence: Synthetic polypeptide
EVQLVESGGGLVQPGGSLRLSCAASGFTFSSYAMSWVRQAPGKGLEWVSAISGSGGSTYYADSVKGRFTISRDNSKNTLYLQMNSLRAEDTAVYYCAKVEGSGSLDYWGQGTLVTVSSGGGGSGGGGSGGGGSEIVMTQSPGTLSLSPGERATLSCRASQSVSSAYLAWYQQKPGQPPRLLISGASTRATGIPDRFGGSGSGTDFTLTISRLEPEDFAVYYCQHYGSSFNGSSLFTFGQGTRLEIK

SEQ ID NO: 316 - protein, 118 aa, synthetic construct
  REGION 1..118: note = Description of Artificial Sequence: Synthetic polypeptide
EVQLVETGGGLVQPGGSLRLSCAASGITFSRYPMSWVRQAPGKGLEWVSGISDSGVSTYYADSAKGRFTISRDNSKNTLFLQMSSLRDEDTAVYYCVTRAGSEASDIWGQGTMVTVSS

SEQ ID NO: 317 - protein, 108 aa, synthetic construct
  REGION 1..108: note = Description of Artificial Sequence: Synthetic polypeptide
EIVLTQSPATLSLSPGERATLSCRASQSVSNSLAWYQQKPGQAPRLLIYDASSRATGIPDRFSGSGSGTDFTLTISRLEPEDFAIYYCQQFGTSSGLTFGGGTKLEIK

SEQ ID NO: 318 - protein, 241 aa, synthetic construct
  REGION 1..241: note = Description of Artificial Sequence: Synthetic polypeptide
EVQLVETGGGLVQPGGSLRLSCAASGITFSRYPMSWVRQAPGKGLEWVSGISDSGVSTYYADSAKGRFTISRDNSKNTLFLQMSSLRDEDTAVYYCVTRAGSEASDIWGQGTMVTVSSGGGGSGGGGSGGGGSEIVLTQSPATLSLSPGERATLSCRASQSVSNSLAWYQQKPGQAPRLLIYDASSRATGIPDRFSGSGSGTDFTLTISRLEPEDFAIYYCQQFGTSSGLTFGGGTKLEIK

SEQ ID NO: 319 - protein, 124 aa, synthetic construct
  REGION 1..124: note = Description of Artificial Sequence: Synthetic polypeptide
QVQLVESGGGLVQPGGSLRLSCAASGFTFSSYAMSWVRQAPGKGLEWVSAISGSGGSTYYADSVKGRFTISRDNAKNSLYLQMNSLRAEDTAIYYCARATYKRELRYYYGMDVWGQGTMVTVSS

SEQ ID NO: 320 - protein, 109 aa, synthetic construct
  REGION 1..109: note = Description of Artificial Sequence: Synthetic polypeptide
EIVMTQSPGTVSLSPGERATLSCRASQSVSSSFLAWYQQKPGQAPRLLIYGASSRATGIPDRFSGSGSGTDFTLTISRLEPEDSAVYYCQQYHSSPSWTFGQGTRLEIK

SEQ ID NO: 321 - protein, 248 aa, synthetic construct
  REGION 1..248: note = Description of Artificial Sequence: Synthetic polypeptide
QVQLVESGGGLVQPGGSLRLSCAASGFTFSSYAMSWVRQAPGKGLEWVSAISGSGGSTYYADSVKGRFTISRDNAKNSLYLQMNSLRAEDTAIYYCARATYKRELRYYYGMDVWGQGTMVTVSSGGGGSGGGGSGGGGSEIVMTQSPGTVSLSPGERATLSCRASQSVSSSFLAWYQQKPGQAPRLLIYGASSRATGIPDRFSGSGSGTDFTLTISRLEPEDSAVYYCQQYHSSPSWTFGQGTRLEIK

SEQ ID NO: 322 - protein, 124 aa, synthetic construct
  REGION 1..124: note = Description of Artificial Sequence: Synthetic polypeptide
EVQLVETGGGLVQPGGSLRLSCAASGFTFSSYAMSWVRQAPGKGLEWVSAISGSGGSTYYADSVKGRFTISRDNSKNTLYLQMNTLKAEDTAVYYCARATYKRELRYYYGMDVWGQGTTVTVSS

SEQ ID NO: 323 - protein, 109 aa, synthetic construct
  REGION 1..109: note = Description of Artificial Sequence: Synthetic polypeptide
EIVLTQSPSTLSLSPGESATLSCRASQSVSTTFLAWYQQKPGQAPRLLIYGSSNRATGIPDRFSGSGSGTDFTLTIRRLEPEDFAVYYCQQYHSSPSWTFGQGTKVEIK

SEQ ID NO: 324 - protein, 248 aa, synthetic construct
  REGION 1..248: note = Description of Artificial Sequence: Synthetic polypeptide
EVQLVETGGGLVQPGGSLRLSCAASGFTFSSYAMSWVRQAPGKGLEWVSAISGSGGSTYYADSVKGRFTISRDNSKNTLYLQMNTLKAEDTAVYYCARATYKRELRYYYGMDVWGQGTTVTVSSGGGGSGGGGSGGGGSEIVLTQSPSTLSLSPGESATLSCRASQSVSTTFLAWYQQKPGQAPRLLIYGSSNRATGIPDRFSGSGSGTDFTLTIRRLEPEDFAVYYCQQYHSSPSWTFGQGTKVEIK

SEQ ID NO: 325 - protein, 117 aa, synthetic construct
  REGION 1..117: note = Description of Artificial Sequence: Synthetic polypeptide
EVQLVETGGGLVQPGRSLRLSCAASGFTFDDYAMHWVRQAPGKGLEWVSGISWNSGSIGYADSVKGRFTISRDNAKNSLYLQMNSLRDEDTAVYYCARVGKAVPDVWGQGTTVTVSS

SEQ ID NO: 326 - protein, 107 aa, synthetic construct
  REGION 1..107: note = Description of Artificial Sequence: Synthetic polypeptide
DIVMTQTPSSLSASVGDRVTITCRASQSISSYLNWYQQKPGKAPKLLIYAASSLQSGVPSRFSGSGSGTDFTLTISSLQPEDFATYYCQQSYSTPYSFGQGTRLEIK

SEQ ID NO: 327 - protein, 239 aa, synthetic construct
  REGION 1..239: note = Description of Artificial Sequence: Synthetic polypeptide
EVQLVETGGGLVQPGRSLRLSCAASGFTFDDYAMHWVRQAPGKGLEWVSGISWNSGSIGYADSVKGRFTISRDNAKNSLYLQMNSLRDEDTAVYYCARVGKAVPDVWGQGTTVTVSSGGGGSGGGGSGGGGSDIVMTQTPSSLSASVGDRVTITCRASQSISSYLNWYQQKPGKAPKLLIYAASSLQSGVPSRFSGSGSGTDFTLTISSLQPEDFATYYCQQSYSTPYSFGQGTRLEIK

SEQ ID NO: 328 - protein, 122 aa, synthetic construct
  REGION 1..122: note = Description of Artificial Sequence: Synthetic polypeptide
EVQLVESGGGLVQPGRSLRLSCTASGFTFDDYAMHWVRQRPGKGLEWVASINWKGNSLAYGDSVKGRFAISRDNAKNTVFLQMNSLRTEDTAVYYCASHQGVAYYNYAMDVWGRGTLVTVSS

SEQ ID NO: 329 - protein, 109 aa, synthetic construct
  REGION 1..109: note = Description of Artificial Sequence: Synthetic polypeptide
EIVLTQSPGTLSLSPGERATLSCRATQSIGSSFLAWYQQRPGQAPRLLIYGASQRATGIPDRFSGRGSGTDFTLTISRVEPEDSAVYYCQHYESSPSWTFGQGTKVEIK

SEQ ID NO: 330 - protein, 246 aa, synthetic construct
  REGION 1..246: note = Description of Artificial Sequence: Synthetic polypeptide
EVQLVESGGGLVQPGRSLRLSCTASGFTFDDYAMHWVRQRPGKGLEWVASINWKGNSLAYGDSVKGRFAISRDNAKNTVFLQMNSLRTEDTAVYYCASHQGVAYYNYAMDVWGRGTLVTVSSGGGGSGGGGSGGGGSEIVLTQSPGTLSLSPGERATLSCRATQSIGSSFLAWYQQRPGQAPRLLIYGASQRATGIPDRFSGRGSGTDFTLTISRVEPEDSAVYYCQHYESSPSWTFGQGTKVEIK

SEQ ID NO: 331 - protein, 117 aa, synthetic construct
  REGION 1..117: note = Description of Artificial Sequence: Synthetic polypeptide
EVQLVESGGGLVQPGGSLRLSCAASGFTFSSYAMSWVRQAPGKGLEWVSAISGSGGSTYYADSVKGRFTISRDNSKNTLYLQMNSLRAEDTAVYYCAKVVRDGMDVWGQGTTVTVSS

SEQ ID NO: 332 - protein, 109 aa, synthetic construct
  REGION 1..109: note = Description of Artificial Sequence: Synthetic polypeptide
EIVLTQSPATLSLSPGERATLSCRASQSVSSSYLAWYQQKPGQAPRLLIYGASSRATGIPDRFSGNGSGTDFTLTISRLEPEDFAVYYCQQYGSPPRFTFGPGTKVDIK

SEQ ID NO: 333 - protein, 241 aa, synthetic construct
  REGION 1..241: note = Description of Artificial Sequence: Synthetic polypeptide
EVQLVESGGGLVQPGGSLRLSCAASGFTFSSYAMSWVRQAPGKGLEWVSAISGSGGSTYYADSVKGRFTISRDNSKNTLYLQMNSLRAEDTAVYYCAKVVRDGMDVWGQGTTVTVSSGGGGSGGGGSGGGGSEIVLTQSPATLSLSPGERATLSCRASQSVSSSYLAWYQQKPGQAPRLLIYGASSRATGIPDRFSGNGSGTDFTLTISRLEPEDFAVYYCQQYGSPPRFTFGPGTKVDIK

SEQ ID NO: 334 - protein, 118 aa, synthetic construct
  REGION 1..118: note = Description of Artificial Sequence: Synthetic polypeptide
EVQLLESGGGLVQPGGSLRLSCAASGFTFSSYAMSWVRQAPGKGLEWVSAISGSGGSTYYADSVKGRFTISRDNSKNTLYLQMNSLRAEDTAVYYCAKIPQTGTFDYWGQGTLVTVSS

SEQ ID NO: 335 - protein, 109 aa, synthetic construct
  REGION 1..109: note = Description of Artificial Sequence: Synthetic polypeptide
EIVLTQSPGTLSLSPGERATLSCRASQSVSSSYLAWYQQRPGQAPRLLIYGASSRATGIPDRFSGSGSGTDFTLTISRLEPEDFAVYYCQHYGSSPSWTFGQGTRLEIK

SEQ ID NO: 336 - protein, 242 aa, synthetic construct
  REGION 1..242: note = Description of Artificial Sequence: Synthetic polypeptide
EVQLLESGGGLVQPGGSLRLSCAASGFTFSSYAMSWVRQAPGKGLEWVSAISGSGGSTYYADSVKGRFTISRDNSKNTLYLQMNSLRAEDTAVYYCAKIPQTGTFDYWGQGTLVTVSSGGGGSGGGGSGGGGSEIVLTQSPGTLSLSPGERATLSCRASQSVSSSYLAWYQQRPGQAPRLLIYGASSRATGIPDRFSGSGSGTDFTLTISRLEPEDFAVYYCQHYGSSPSWTFGQGTRLEIK

SEQ ID NO: 337 - protein, 124 aa, synthetic construct
  REGION 1..124: note = Description of Artificial Sequence: Synthetic polypeptide
EVQLVETGGGLVQPGGSLRLSCAASGFTFSSYAMSWVRQAPGKGLEWVSAISGSGGSTYYADSVKGRFTMSRENDKNSVFLQMNSLRVEDTGVYYCARANYKRELRYYYGMDVWGQGTMVTVSS

SEQ ID NO: 338 - protein, 109 aa, synthetic construct
  REGION 1..109: note = Description of Artificial Sequence: Synthetic polypeptide
EIVMTQSPGTLSLSPGESATLSCRASQRVASNYLAWYQHKPGQAPSLLISGASSRATGVPDRFSGSGSGTDFTLAISRLEPEDSAVYYCQHYDSSPSWTFGQGTKVEIK

SEQ ID NO: 339 - protein, 248 aa, synthetic construct
  REGION 1..248: note = Description of Artificial Sequence: Synthetic polypeptide
EVQLVETGGGLVQPGGSLRLSCAASGFTFSSYAMSWVRQAPGKGLEWVSAISGSGGSTYYADSVKGRFTMSRENDKNSVFLQMNSLRVEDTGVYYCARANYKRELRYYYGMDVWGQGTMVTVSSGGGGSGGGGSGGGGSEIVMTQSPGTLSLSPGESATLSCRASQRVASNYLAWYQHKPGQAPSLLISGASSRATGVPDRFSGSGSGTDFTLAISRLEPEDSAVYYCQHYDSSPSWTFGQGTKVEIK

SEQ ID NO: 340 - protein, 120 aa, synthetic construct
  REGION 1..120: note = Description of Artificial Sequence: Synthetic polypeptide
EVQLLETGGGLVQPGGSLRLSCAASGFSFSSYAMSWVRQAPGKGLEWVSAISGSGGSTYYADSVKGRFTISRDNSKNTLYLQMNSLRAEDTAVYYCAKALVGATGAFDIWGQGTLVTVSS

SEQ ID NO: 341 - protein, 109 aa, synthetic construct
  REGION 1..109: note = Description of Artificial Sequence: Synthetic polypeptide
EIVLTQSPGTLSLSPGERATLSCRASQSLSSNFLAWYQQKPGQAPGLLIYGASNWATGTPDRFSGSGSGTDFTLTITRLEPEDFAVYYCQYYGTSPMYTFGQGTKVEIK

SEQ ID NO: 342 - protein, 244 aa, synthetic construct
  REGION 1..244: note = Description of Artificial Sequence: Synthetic polypeptide
EVQLLETGGGLVQPGGSLRLSCAASGFSFSSYAMSWVRQAPGKGLEWVSAISGSGGSTYYADSVKGRFTISRDNSKNTLYLQMNSLRAEDTAVYYCAKALVGATGAFDIWGQGTLVTVSSGGGGSGGGGSGGGGSEIVLTQSPGTLSLSPGERATLSCRASQSLSSNFLAWYQQKPGQAPGLLIYGASNWATGTPDRFSGSGSGTDFTLTITRLEPEDFAVYYCQYYGTSPMYTFGQGTKVEIK

SEQ ID NO: 343 - protein, 117 aa, synthetic construct
  REGION 1..117: note = Description of Artificial Sequence: Synthetic polypeptide
EVQLLESGGGLVQPGGSLRLSCAASGFTFSSYAMSWVRQAPGKGLEWVSAISGSGGSTYYADSVKGRFTISRDNSKNTLYLQMNSLRAEDTAVYYCVLWFGEGFDPWGQGTLVTVSS

SEQ ID NO: 344 - protein, 112 aa, synthetic construct
  REGION 1..112: note = Description of Artificial Sequence: Synthetic polypeptide
DIVLTQSPLSLPVTPGEPASISCRSSQSLLHSNGYNYLDWYLQKPGQSPQLLIYLGSNRASGVPDRFSGSGSGTDFTLKISRVEAEDVGVYYCMQALQTPLTFGGGTKVDIK

SEQ ID NO: 345 - protein, 244 aa, synthetic construct
  REGION 1..244: note = Description of Artificial Sequence: Synthetic polypeptide
EVQLLESGGGLVQPGGSLRLSCAASGFTFSSYAMSWVRQAPGKGLEWVSAISGSGGSTYYADSVKGRFTISRDNSKNTLYLQMNSLRAEDTAVYYCVLWFGEGFDPWGQGTLVTVSSGGGGSGGGGSGGGGSDIVLTQSPLSLPVTPGEPASISCRSSQSLLHSNGYNYLDWYLQKPGQSPQLLIYLGSNRASGVPDRFSGSGSGTDFTLKISRVEAEDVGVYYCMQALQTPLTFGGGTKVDIK

SEQ ID NO: 346 - protein, 126 aa, synthetic construct
  REGION 1..126: note = Description of Artificial Sequence: Synthetic polypeptide
QVQLVESGGGLVQPGGSLRLSCAASGFTFSSYAMSWVRQAPGKGLEWVSAISGSGGSTYYADSVKGRFTISRDNSKNTLYLQMNSLRAEDTAVYYCAKVGYDSSGYYRDYYGMDVWGQGTTVTVSS

SEQ ID NO: 347 - protein, 110 aa, synthetic construct
  REGION 1..110: note = Description of Artificial Sequence: Synthetic polypeptide
EIVLTQSPGTLSLSPGERATLSCRASQSVSSSYLAWYQQKPGQAPRLLIYGTSSRATGISDRFSGSGSGTDFTLTISRLEPEDFAVYYCQHYGNSPPKFTFGPGTKLEIK

SEQ ID NO: 348 - protein, 251 aa, synthetic construct
  REGION 1..251: note = Description of Artificial Sequence: Synthetic polypeptide
QVQLVESGGGLVQPGGSLRLSCAASGFTFSSYAMSWVRQAPGKGLEWVSAISGSGGSTYYADSVKGRFTISRDNSKNTLYLQMNSLRAEDTAVYYCAKVGYDSSGYYRDYYGMDVWGQGTTVTVSSGGGGSGGGGSGGGGSEIVLTQSPGTLSLSPGERATLSCRASQSVSSSYLAWYQQKPGQAPRLLIYGTSSRATGISDRFSGSGSGTDFTLTISRLEPEDFAVYYCQHYGNSPPKFTFGPGTKLEIK

SEQ ID NO: 349 - protein, 122 aa, synthetic construct
  REGION 1..122: note = Description of Artificial Sequence: Synthetic polypeptide
EVQLVESGGGLVQPGGSLRLSCAASGFTFSSYAMSWVRQAPGKGLEWVSAISGSGGSTYYADSVKGRFTISRDNSKNTLYLQMNSLRAEDTAVYYCAKMGWSSGYLGAFDIWGQGTTVTVSS

SEQ ID NO: 350 - protein, 109 aa, synthetic construct
  REGION 1..109: note = Description of Artificial Sequence: Synthetic polypeptide
EIVLTQSPGTLSLSPGERATLSCRASQSVASSFLAWYQQKPGQAPRLLIYGASGRATGIPDRFSGSGSGTDFTLTISRLEPEDFAVYYCQHYGGSPRLTFGGGTKVDIK

SEQ ID NO: 351 - protein, 246 aa, synthetic construct
  REGION 1..246: note = Description of Artificial Sequence: Synthetic polypeptide
EVQLVESGGGLVQPGGSLRLSCAASGFTFSSYAMSWVRQAPGKGLEWVSAISGSGGSTYYADSVKGRFTISRDNSKNTLYLQMNSLRAEDTAVYYCAKMGWSSGYLGAFDIWGQGTTVTVSSGGGGSGGGGSGGGGSEIVLTQSPGTLSLSPGERATLSCRASQSVASSFLAWYQQKPGQAPRLLIYGASGRATGIPDRFSGSGSGTDFTLTISRLEPEDFAVYYCQHYGGSPRLTFGGGTKVDIK

SEQ ID NO: 352 - protein, 122 aa, synthetic construct
  REGION 1..122: note = Description of Artificial Sequence: Synthetic polypeptide
QIQLVQSGPDLKKPGETVKLSCKASGYTFTNFGMNWVKQAPGKGFKWMAWINTYTGESYFADDFKGRFAFSVETSATTAYLQINNLKTEDTATYFCARGEIYYGYDGGFAYWGQGTLVTVSA

SEQ ID NO: 353 - protein, 107 aa, synthetic construct
  REGION 1..107: note = Description of Artificial Sequence: Synthetic polypeptide
DVVMTQSHRFMSTSVGDRVSITCRASQDVNTAVSWYQQKPGQSPKLLIFSASYRYTGVPDRFTGSGSGADFTLTISSVQAEDLAVYYCQQHYSTPWTFGGGTKLDIK

SEQ ID NO: 354 - protein, 244 aa, synthetic construct
  REGION 1..244: note = Description of Artificial Sequence: Synthetic polypeptide
QIQLVQSGPDLKKPGETVKLSCKASGYTFTNFGMNWVKQAPGKGFKWMAWINTYTGESYFADDFKGRFAFSVETSATTAYLQINNLKTEDTATYFCARGEIYYGYDGGFAYWGQGTLVTVSAGGGGSGGGGSGGGGSDVVMTQSHRFMSTSVGDRVSITCRASQDVNTAVSWYQQKPGQSPKLLIFSASYRYTGVPDRFTGSGSGADFTLTISSVQAEDLAVYYCQQHYSTPWTFGGGTKLDIK

SEQ ID NO: 355 - protein, 117 aa, synthetic construct
  REGION 1..117: note = Description of Artificial Sequence: Synthetic polypeptide
QIQLVQSGPELKKPGETVKISCKASGYTFTDYSINWVKRAPGKGLKWMGWINTETREPAYAYDFRGRFAFSLETSASTAYLQINNLKYEDTATYFCALDYSYAMDYWGQGTSVTVSS

SEQ ID NO: 356 - protein, 111 aa, synthetic construct
  REGION 1..111: note = Description of Artificial Sequence: Synthetic polypeptide
DIVLTQSPASLAMSLGKRATISCRASESVSVIGAHLIHWYQQKPGQPPKLLIYLASNLETGVPARFSGSGSGTDFTLTIDPVEEDDVAIYSCLQSRIFPRTFGGGTKLEIK

SEQ ID NO: 357 - protein, 249 aa, synthetic construct
  REGION 1..249: note = Description of Artificial Sequence: Synthetic polypeptide
QIQLVQSGPELKKPGETVKISCKASGYTFTDYSINWVKRAPGKGLKWMGWINTETREPAYAYDFRGRFAFSLETSASTAYLQINNLKYEDTATYFCALDYSYAMDYWGQGTSVTVSSGGGGSGGGGSGGGGSQIQLVQSGPELKKPGETVKISCKASGYTFTDYSINWVKRAPGKGLKWMGWINTETREPAYAYDFRGRFAFSLETSASTAYLQINNLKYEDTATYFCALDYSYAMDYWGQGTSVTVSS

SEQ ID NO: 358 - protein, 117 aa, synthetic construct
  REGION 1..117: note = Description of Artificial Sequence: Synthetic polypeptide
QIQLVQSGPELKKPGETVKISCKASGYTFRHYSMNWVKQAPGKGLKWMGRINTESGVPIYADDFKGRFAFSVETSASTAYLVINNLKDEDTASYFCSNDYLYSLDFWGQGTALTVSS

SEQ ID NO: 359 - protein, 111 aa, synthetic construct
  REGION 1..111: note = Description of Artificial Sequence: Synthetic polypeptide
DIVLTQSPPSLAMSLGKRATISCRASESVTILGSHLIYWYQQKPGQPPTLLIQLASNVQTGVPARFSGSGSRTDFTLTIDPVEEDDVAVYYCLQSRTIPRTFGGGTKLEIK

SEQ ID NO: 360 - protein, 243 aa, synthetic construct
  REGION 1..243: note = Description of Artificial Sequence: Synthetic polypeptide
QIQLVQSGPELKKPGETVKISCKASGYTFRHYSMNWVKQAPGKGLKWMGRINTESGVPIYADDFKGRFAFSVETSASTAYLVINNLKDEDTASYFCSNDYLYSLDFWGQGTALTVSSGGGGSGGGGSGGGGSDIVLTQSPPSLAMSLGKRATISCRASESVTILGSHLIYWYQQKPGQPPTLLIQLASNVQTGVPARFSGSGSRTDFTLTIDPVEEDDVAVYYCLQSRTIPRTFGGGTKLEIK

SEQ ID NO: 361 - protein, 117 aa, synthetic construct
  REGION 1..117: note = Description of Artificial Sequence: Synthetic polypeptide
QIQLVQSGPELKKPGETVKISCKASGYTFTHYSMNWVKQAPGKGLKWMGRINTETGEPLYADDFKGRFAFSLETSASTAYLVINNLKNEDTATFFCSNDYLYSCDYWGQGTTLTVSS

SEQ ID NO: 362 - protein, 243 aa, synthetic construct
  REGION 1..243: note = Description of Artificial Sequence: Synthetic polypeptide
QIQLVQSGPELKKPGETVKISCKASGYTFTHYSMNWVKQAPGKGLKWMGRINTETGEPLYADDFKGRFAFSLETSASTAYLVINNLKNEDTATFFCSNDYLYSCDYWGQGTTLTVSSGGGGSGGGGSGGGGSDIVLTQSPPSLAMSLGKRATISCRASESVTILGSHLIYWYQQKPGQPPTLLIQLASNVQTGVPARFSGSGSRTDFTLTIDPVEEDDVAVYYCLQSRTIPRTFGGGTKLEIK

SEQ ID NO: 363 - protein, 11 aa, synthetic construct
  REGION 1..11: note = Description of Artificial Sequence: Synthetic peptide
RASQSISSYLN

SEQ ID NO: 364 - protein, 12 aa, synthetic construct
  REGION 1..12: note = Description of Artificial Sequence: Synthetic peptide
RASQSISSSFLA

SEQ ID NO: 365 - protein, 16 aa, synthetic construct
  REGION 1..16: note = Description of Artificial Sequence: Synthetic peptide
RSSQSLLHSNGYNYLD

SEQ ID NO: 366 - protein, 16 aa, synthetic construct
  REGION 1..16: note = Description of Artificial Sequence: Synthetic peptide
KSSQSLLRNDGKTPLY

SEQ ID NO: 367 - protein, 16 aa, synthetic construct
  REGION 1..16: note = Description of Artificial Sequence: Synthetic peptide
RSSQSLLHSNGYNYLN

SEQ ID NO: 368 - protein, 16 aa, synthetic construct
  REGION 1..16: note = Description of Artificial Sequence: Synthetic peptide
RSSQSLLYSNGYNYVD

SEQ ID NO: 369 - protein, 11 aa, synthetic construct
  REGION 1..11: note = Description of Artificial Sequence: Synthetic peptide
RASQSVSSNLA

SEQ ID NO: 370 - protein, 11 aa, synthetic construct
  REGION 1..11: note = Description of Artificial Sequence: Synthetic peptide
RASQSVSSKLA

SEQ ID NO: 371 - protein, 12 aa, synthetic construct
  REGION 1..12: note = Description of Artificial Sequence: Synthetic peptide
RASQSVGSTNLA

SEQ ID NO: 372 - protein, 16 aa, synthetic construct
  REGION 1..16: note = Description of Artificial Sequence: Synthetic peptide
KSSESLVHNSGKTYLN

SEQ ID NO: 373 - protein, 11 aa, synthetic construct
  REGION 1..11: note = Description of Artificial Sequence: Synthetic peptide
QASEDINKFLN

SEQ ID NO: 374 - protein, 11 aa, synthetic construct
  REGION 1..11: note = Description of Artificial Sequence: Synthetic peptide
RASQSVGSNLA

SEQ ID NO: 375 - protein, 12 aa, synthetic construct
  REGION 1..12: note = Description of Artificial Sequence: Synthetic peptide
RASQSIGSSSLA

SEQ ID NO: 376 - protein, 11 aa, synthetic construct
  REGION 1..11: note = Description of Artificial Sequence: Synthetic peptide
KASQDIDDAMN

SEQ ID NO: 377 - protein, 11 aa, synthetic construct
  REGION 1..11: note = Description of Artificial Sequence: Synthetic peptide
RASQDIYNNLA

SEQ ID NO: 378 - protein, 11 aa, synthetic construct
  REGION 1..11: note = Description of Artificial Sequence: Synthetic peptide
GGNNIGTKSVH

SEQ ID NO: 379 - protein, 11 aa, synthetic construct
  REGION 1..11: note = Description of Artificial Sequence: Synthetic peptide
SGDGLSKKYVS

SEQ ID NO: 380 - protein, 11 aa, synthetic construct
  REGION 1..11: note = Description of Artificial Sequence: Synthetic peptide
RASQGIRNWLA

SEQ ID NO: 381 - protein, 11 aa, synthetic construct
  REGION 1..11: note = Description of Artificial Sequence: Synthetic peptide
GGNNIGSKSVH

SEQ ID NO: 382 - protein, 11 aa, synthetic construct
  REGION 1..11: note = Description of Artificial Sequence: Synthetic peptide
QGDSLGNYYAT

SEQ ID NO: 383 - protein, 12 aa, synthetic construct
  REGION 1..12: note = Description of Artificial Sequence: Synthetic peptide
RASQSVSSAYLA

SEQ ID NO: 384 - protein, 11 aa, synthetic construct
  REGION 1..11: note = Description of Artificial Sequence: Synthetic peptide
RASQSVSNSLA

SEQ ID NO: 385 - protein, 12 aa, synthetic construct
  REGION 1..12: note = Description of Artificial Sequence: Synthetic peptide
RASQSVSSSFLA

SEQ ID NO: 386 - protein, 12 aa, synthetic construct
  REGION 1..12: note = Description of Artificial Sequence: Synthetic peptide
RASQSVSTTFLA

SEQ ID NO: 387 - protein, 12 aa, synthetic construct
  REGION 1..12: note = Description of Artificial Sequence: Synthetic peptide
RATQSIGSSFLA

SEQ ID NO: 388 - protein, 12 aa, synthetic construct
  REGION 1..12: note = Description of Artificial Sequence: Synthetic peptide
RASQSVSSSYLA

SEQ ID NO: 389 - protein, 12 aa, synthetic construct
  REGION 1..12: note = Description of Artificial Sequence: Synthetic peptide
RASQRVASNYLA

SEQ ID NO: 390 - protein, 12 aa, synthetic construct
  REGION 1..12: note = Description of Artificial Sequence: Synthetic peptide
RASQSLSSNFLA

SEQ ID NO: 391 - protein, 12 aa, synthetic construct
  REGION 1..12: note = Description of Artificial Sequence: Synthetic peptide
RASQSVASSFLA

SEQ ID NO: 392 - protein, 11 aa, synthetic construct
  REGION 1..11: note = Description of Artificial Sequence: Synthetic peptide
RASQDVNTAVS

SEQ ID NO: 393 - protein, 15 aa, synthetic construct
  REGION 1..15: note = Description of Artificial Sequence: Synthetic peptide
RASESVSVIGAHLIH

SEQ ID NO: 394 - protein, 15 aa, synthetic construct
  REGION 1..15: note = Description of Artificial Sequence: Synthetic peptide
RASESVTILGSHLIY

SEQ ID NO: 395 - protein, 7 aa, synthetic construct
  REGION 1..7: note = Description of Artificial Sequence: Synthetic peptide
AASSLQS

SEQ ID NO: 396 - protein, 7 aa, synthetic construct
  REGION 1..7: note = Description of Artificial Sequence: Synthetic peptide
GASRRAT

SEQ ID NO: 397 - protein, 7 aa, synthetic construct
  REGION 1..7: note = Description of Artificial Sequence: Synthetic peptide
LGSNRAS

SEQ ID NO: 398 - protein, 7 aa, synthetic construct
  REGION 1..7: note = Description of Artificial Sequence: Synthetic peptide
EVSNRFS

SEQ ID NO: 399 - protein, 7 aa, synthetic construct
  REGION 1..7: note = Description of Artificial Sequence: Synthetic peptide
LGSKRAS

SEQ ID NO: 400 - protein, 7 aa, synthetic construct
  REGION 1..7: note = Description of Artificial Sequence: Synthetic peptide
GASTLAS

SEQ ID NO: 401 - protein, 7 aa, synthetic construct
  REGION 1..7: note = Description of Artificial Sequence: Synthetic peptide
GASTRAS

SEQ ID NO: 402 - protein, 7 aa, synthetic construct
  REGION 1..7: note = Description of Artificial Sequence: Synthetic peptide
GASIRAT

SEQ ID NO: 403 - protein, 7 aa, synthetic construct
  REGION 1..7: note = Description of Artificial Sequence: Synthetic peptide
EVSNRDS

SEQ ID NO: 404 - protein, 7 aa, synthetic construct
  REGION 1..7: note = Description of Artificial Sequence: Synthetic peptide
DASTLQT

SEQ ID NO: 405 - protein, 7 aa, synthetic construct
  REGION 1..7: note = Description of Artificial Sequence: Synthetic peptide
GASTRAT

SEQ ID NO: 406 - protein, 7 aa, synthetic construct
  REGION 1..7: note = Description of Artificial Sequence: Synthetic peptide
GASSRAS

SEQ ID NO: 407 - protein, 7 aa, synthetic construct
  REGION 1..7: note = Description of Artificial Sequence: Synthetic peptide
SATSPVP

SEQ ID NO: 408 - protein, 7 aa, synthetic construct
  REGION 1..7: note = Description of Artificial Sequence: Synthetic peptide
AANKSQS

SEQ ID NO: 409 - protein, 7 aa, synthetic construct
  REGION 1..7: note = Description of Artificial Sequence: Synthetic peptide
DDSVRPS

SEQ ID NO: 410 - protein, 7 aa, synthetic construct
  REGION 1..7: note = Description of Artificial Sequence: Synthetic peptide
RDKERPS

SEQ ID NO: 411 - protein, 7 aa, synthetic construct
  REGION 1..7: note = Description of Artificial Sequence: Synthetic peptide
AASNLQS

SEQ ID NO: 412 - protein, 7 aa, synthetic construct
  REGION 1..7: note = Description of Artificial Sequence: Synthetic peptide
GKNNRPS

SEQ ID NO: 413 - protein, 7 aa, synthetic construct
  REGION 1..7: note = Description of Artificial Sequence: Synthetic peptide
GTNNRPS

SEQ ID NO: 414 - protein, 7 aa, synthetic construct
  REGION 1..7: note = Description of Artificial Sequence: Synthetic peptide
DASSRAT

SEQ ID NO: 415 - protein, 7 aa, synthetic construct
  REGION 1..7: note = Description of Artificial Sequence: Synthetic peptide
GASSRAT

SEQ ID NO: 416 - protein, 7 aa, synthetic construct
  REGION 1..7: note = Description of Artificial Sequence: Synthetic peptide
GSSNRAT

SEQ ID NO: 417 - protein, 7 aa, synthetic construct
  REGION 1..7: note = Description of Artificial Sequence: Synthetic peptide
GASQRAT

SEQ ID NO: 418 - protein, 7 aa, synthetic construct
  REGION 1..7: note = Description of Artificial Sequence: Synthetic peptide
GASNWAT

SEQ ID NO: 419 - protein, 7 aa, synthetic construct
  REGION 1..7: note = Description of Artificial Sequence: Synthetic peptide
GTSSRAT

SEQ ID NO: 420 - protein, 7 aa, synthetic construct
  REGION 1..7: note = Description of Artificial Sequence: Synthetic peptide
GASGRAT

SEQ ID NO: 421 - protein, 7 aa, synthetic construct
  REGION 1..7: note = Description of Artificial Sequence: Synthetic peptide
SASYRYT

SEQ ID NO: 422 - protein, 7 aa, synthetic construct
  REGION 1..7: note = Description of Artificial Sequence: Synthetic peptide
LASNLET

SEQ ID NO: 423 - protein, 7 aa, synthetic construct
  REGION 1..7: note = Description of Artificial Sequence: Synthetic peptide
LASNVQT

SEQ ID NO: 424 - protein, 9 aa, synthetic construct
  REGION 1..9: note = Description of Artificial Sequence: Synthetic peptide
QQSYSTPYT

SEQ ID NO: 425 - protein, 10 aa, synthetic construct
  REGION 1..10: note = Description of Artificial Sequence: Synthetic peptide
QQYHSSPSWT

SEQ ID NO: 426 - protein, 9 aa, synthetic construct
  REGION 1..9: note = Description of Artificial Sequence: Synthetic peptide
MQALQTPYT

SEQ ID NO: 427 - protein, 8 aa, synthetic construct
  REGION 1..8: note = Description of Artificial Sequence: Synthetic peptide
MQNIQFPS

SEQ ID NO: 428 - protein, 8 aa, synthetic construct
  REGION 1..8: note = Description of Artificial Sequence: Synthetic peptide
QQSYKRAS

SEQ ID NO: 429 - protein, 9 aa, synthetic construct
  REGION 1..9: note = Description of Artificial Sequence: Synthetic peptide
MQGRQFPYS

SEQ ID NO: 430 - protein, 8 aa, synthetic construct
  REGION 1..8: note = Description of Artificial Sequence: Synthetic peptide
QQYGSSLT

SEQ ID NO: 431 - protein, 9 aa, synthetic construct
  REGION 1..9: note = Description of Artificial Sequence: Synthetic peptide
QQYGSSSWT

SEQ ID NO: 432 - protein, 10 aa, synthetic construct
  REGION 1..10: note = Description of Artificial Sequence: Synthetic peptide
QQYGSSPPWT

SEQ ID NO: 433 - protein, 7 aa, synthetic construct
  REGION 1..7: note = Description of Artificial Sequence: Synthetic peptide
QQSYTLA

SEQ ID NO: 434 - protein, 9 aa, synthetic construct
  REGION 1..9: note = Description of Artificial Sequence: Synthetic peptide
MQGTHWPGT

SEQ ID NO: 435 - protein, 9 aa, synthetic construct
  REGION 1..9: note = Description of Artificial Sequence: Synthetic peptide
QQYESLPLT

SEQ ID NO: 436 - protein, 10 aa, synthetic construct
  REGION 1..10: note = Description of Artificial Sequence: Synthetic peptide
QQYNDWLPVT

SEQ ID NO: 437 - protein, 10 aa, synthetic construct
  REGION 1..10: note = Description of Artificial Sequence: Synthetic peptide
QQYAGSPPFT

SEQ ID NO: 438 - protein, 9 aa, synthetic construct
  REGION 1..9: note = Description of Artificial Sequence: Synthetic peptide
LQHDNFPLT

SEQ ID NO: 439 - protein, 9 aa, synthetic construct
  REGION 1..9: note = Description of Artificial Sequence: Synthetic peptide
QHYYRFPYS

SEQ ID NO: 440 - protein, 11 aa, synthetic construct
  REGION 1..11: note = Description of Artificial Sequence: Synthetic peptide
QVWDSDSEHVV

SEQ ID NO: 441 - protein, 9 aa, synthetic construct
  REGION 1..9: note = Description of Artificial Sequence: Synthetic peptide
QAWDDTTVV

SEQ ID NO: 442 - protein, 9 aa, synthetic construct
  REGION 1..9: note = Description of Artificial Sequence: Synthetic peptide
QKYNSAPFT

SEQ ID NO: 443 - protein, 12 aa, synthetic construct
  REGION 1..12: note = Description of Artificial Sequence: Synthetic peptide
SSRDSSGDHLRV

SEQ ID NO: 444 - protein, 11 aa, synthetic construct
  REGION 1..11: note = Description of Artificial Sequence: Synthetic peptide
NSRDSSGHHLL

SEQ ID NO: 445 - protein, 14 aa, synthetic construct
  REGION 1..14: note = Description of Artificial Sequence: Synthetic peptide
QHYGSSFNGSSLFT

SEQ ID NO: 446 - protein, 10 aa, synthetic construct
  REGION 1..10: note = Description of Artificial Sequence: Synthetic peptide
QQFGTSSGLT

SEQ ID NO: 447 - protein, 9 aa, synthetic construct
  REGION 1..9: note = Description of Artificial Sequence: Synthetic peptide
QQSYSTPYS

SEQ ID NO: 448 - protein, 10 aa, synthetic construct
  REGION 1..10: note = Description of Artificial Sequence: Synthetic peptide
QHYESSPSWT

SEQ ID NO: 449 - protein, 10 aa, synthetic construct
  REGION 1..10: note = Description of Artificial Sequence: Synthetic peptide
QQYGSPPRFT

SEQ ID NO: 450 - protein, 10 aa, synthetic construct
  REGION 1..10: note = Description of Artificial Sequence: Synthetic peptide
QHYGSSPSWT

SEQ ID NO: 451 - protein, 10 aa, synthetic construct
  REGION 1..10: note = Description of Artificial Sequence: Synthetic peptide
QHYDSSPSWT

SEQ ID NO: 452 - protein, 10 aa, synthetic construct
  REGION 1..10: note = Description of Artificial Sequence: Synthetic peptide
QYYGTSPMYT

SEQ ID NO: 453 - protein, 9 aa, synthetic construct
  REGION 1..9: note = Description of Artificial Sequence: Synthetic peptide
MQALQTPLT

SEQ ID NO: 454 - protein, 11 aa, synthetic construct
  REGION 1..11: note = Description of Artificial Sequence: Synthetic peptide
QHYGNSPPKFT

SEQ ID NO: 455 - protein, 10 aa, synthetic construct
  REGION 1..10: note = Description of Artificial Sequence: Synthetic peptide
QHYGGSPRLT

SEQ ID NO: 456 - protein, 9 aa, synthetic construct
  REGION 1..9: note = Description of Artificial Sequence: Synthetic peptide
QQHYSTPWT

SEQ ID NO: 457 - protein, 9 aa, synthetic construct
  REGION 1..9: note = Description of Artificial Sequence: Synthetic peptide
LQSRIFPRT

SEQ ID NO: 458 - protein, 9 aa, synthetic construct
  REGION 1..9: note = Description of Artificial Sequence: Synthetic peptide
LQSRTIPRT

SEQ ID NO: 459 - protein, 7 aa, synthetic construct
  REGION 1..7: note = Description of Artificial Sequence: Synthetic peptide
SQSISSY

SEQ ID NO: 460 - protein, 8 aa, synthetic construct
  REGION 1..8: note = Description of Artificial Sequence: Synthetic peptide
SQSISSSF

SEQ ID NO: 461 - protein, 12 aa, synthetic construct
  REGION 1..12: note = Description of Artificial Sequence: Synthetic peptide
SQSLLHSNGYNY

SEQ ID NO: 462 - protein, 12 aa, synthetic construct
  REGION 1..12: note = Description of Artificial Sequence: Synthetic peptide
SQSLLRNDGKTP

SEQ ID NO: 463 - protein, 12 aa, synthetic construct
  REGION 1..12: note = Description of Artificial Sequence: Synthetic peptide
SQSLLYSNGYNY

SEQ ID NO: 464 - protein, 7 aa, synthetic construct
  REGION 1..7: note = Description of Artificial Sequence: Synthetic peptide
SQSVSSN

SEQ ID NO: 465 - protein, 7 aa, synthetic construct
  REGION 1..7: note = Description of Artificial Sequence: Synthetic peptide
SQSVSSK

SEQ ID NO: 466 - protein, 8 aa, synthetic construct
  REGION 1..8: note = Description of Artificial Sequence: Synthetic peptide
SQSVGSTN

SEQ ID NO: 467 - protein, 12 aa, synthetic construct
  REGION 1..12: note = Description of Artificial Sequence: Synthetic peptide
SESLVHNSGKTY

SEQ ID NO: 468 - protein, 7 aa, synthetic construct
  REGION 1..7: note = Description of Artificial Sequence: Synthetic peptide
SEDINKF

SEQ ID NO: 469 - protein, 7 aa, synthetic construct
  REGION 1..7: note = Description of Artificial Sequence: Synthetic peptide
SQSVGSN

SEQ ID NO: 470 - protein, 8 aa, synthetic construct
  REGION 1..8: note = Description of Artificial Sequence: Synthetic peptide
SQSIGSSS

SEQ ID NO: 471 - protein, 7 aa, synthetic construct
  REGION 1..7: note = Description of Artificial Sequence: Synthetic peptide
SQDIDDA

SEQ ID NO: 472 - protein, 7 aa, synthetic construct
  REGION 1..7: note = Description of Artificial Sequence: Synthetic peptide
SQDIYNN

SEQ ID NO: 473 - protein, 7 aa, synthetic construct
  REGION 1..7: note = Description of Artificial Sequence: Synthetic peptide
NNIGTKS

SEQ ID NO: 474 - protein, 7 aa, synthetic construct
  REGION 1..7: note = Description of Artificial Sequence: Synthetic peptide
DGLSKKY

SEQ ID NO: 475 - protein, 7 aa, synthetic construct
  REGION 1..7: note = Description of Artificial Sequence: Synthetic peptide
SQGIRNW

SEQ ID NO: 476 - protein, 7 aa, synthetic construct
  REGION 1..7: note = Description of Artificial Sequence: Synthetic peptide
NNIGSKS

SEQ ID NO: 477 - protein, 7 aa, synthetic construct
  REGION 1..7: note = Description of Artificial Sequence: Synthetic peptide
DSLGNYY

SEQ ID NO: 478 - protein, 8 aa, synthetic construct
  REGION 1..8: note = Description of Artificial Sequence: Synthetic peptide
SQSVSSAY

SEQ ID NO: 479 - protein, 7 aa, synthetic construct
  REGION 1..7: note = Description of Artificial Sequence: Synthetic peptide
SQSVSNS

SEQ ID NO: 480 - protein, 8 aa, synthetic construct
  REGION 1..8: note = Description of Artificial Sequence: Synthetic peptide
SQSVSSSF

SEQ ID NO: 481 - protein, 8 aa, synthetic construct
  REGION 1..8: note = Description of Artificial Sequence: Synthetic peptide
SQSVSTTF

SEQ ID NO: 482 - protein, 8 aa, synthetic construct
  REGION 1..8: note = Description of Artificial Sequence: Synthetic peptide
TQSIGSSF

SEQ ID NO: 483 - protein, 8 aa, synthetic construct
  REGION 1..8: note = Description of Artificial Sequence: Synthetic peptide
SQSVSSSY

SEQ ID NO: 484 - protein, 8 aa, synthetic construct
  REGION 1..8: note = Description of Artificial Sequence: Synthetic peptide
SQRVASNY

SEQ ID NO: 485 - protein, 8 aa, synthetic construct
  REGION 1..8: note = Description of Artificial Sequence: Synthetic peptide
SQSLSSNF

SEQ ID NO: 486 - protein, 8 aa, synthetic construct
  REGION 1..8: note = Description of Artificial Sequence: Synthetic peptide
SQSVASSF

SEQ ID NO: 487 - protein, 7 aa, synthetic construct
  REGION 1..7: note = Description of Artificial Sequence: Synthetic peptide
SQDVNTA

SEQ ID NO: 488 - protein, 11 aa, synthetic construct
  REGION 1..11: note = Description of Artificial Sequence: Synthetic peptide
SESVSVIGAHL

SEQ ID NO: 489 - protein, 11 aa, synthetic construct
  REGION 1..11: note = Description of Artificial Sequence: Synthetic peptide
SESVTILGSHL

SEQ ID NO: 490-502 - skipped (13 entries)

SEQ ID NO: 503 - protein, 6 aa, synthetic construct
  REGION 1..6: note = Description of Artificial Sequence: Synthetic peptide
SYSTPY

SEQ ID NO: 504 - protein, 7 aa, synthetic construct
  REGION 1..7: note = Description of Artificial Sequence: Synthetic peptide
YHSSPSW

SEQ ID NO: 505 - protein, 6 aa, synthetic construct
  REGION 1..6: note = Description of Artificial Sequence: Synthetic peptide
ALQTPY

SEQ ID NO: 506 - protein, 5 aa, synthetic construct
  REGION 1..5: note = Description of Artificial Sequence: Synthetic peptide
NIQFP

SEQ ID NO: 507 - protein, 5 aa, synthetic construct
  REGION 1..5: note = Description of Artificial Sequence: Synthetic peptide
SYKRA

SEQ ID NO: 508 - protein, 6 aa, synthetic construct
  REGION 1..6: note = Description of Artificial Sequence: Synthetic peptide
GRQFPY

SEQ ID NO: 509 - protein, 5 aa, synthetic construct
  REGION 1..5: note = Description of Artificial Sequence: Synthetic peptide
YGSSL

SEQ ID NO: 510 - protein, 6 aa, synthetic construct
  REGION 1..6: note = Description of Artificial Sequence: Synthetic peptide
YGSSSW

SEQ ID NO: 511 - protein, 7 aa, synthetic construct
  REGION 1..7: note = Description of Artificial Sequence: Synthetic peptide
YGSSPPW

SEQ ID NO: 512 - protein, 4 aa, synthetic construct
  REGION 1..4: note = Description of Artificial Sequence: Synthetic peptide
SYTL

SEQ ID NO: 513 - protein, 6 aa, synthetic construct
  REGION 1..6: note = Description of Artificial Sequence: Synthetic peptide
GTHWPG

SEQ ID NO: 514 - protein, 6 aa, synthetic construct
  REGION 1..6: note = Description of Artificial Sequence: Synthetic peptide
YESLPL

SEQ ID NO: 515 - protein, 7 aa, synthetic construct
  REGION 1..7: note = Description of Artificial Sequence: Synthetic peptide
YNDWLPV

SEQ ID NO: 516 - protein, 7 aa, synthetic construct
  REGION 1..7: note = Description of Artificial Sequence: Synthetic peptide
YAGSPPF

SEQ ID NO: 517 - protein, 6 aa, synthetic construct
  REGION 1..6: note = Description of Artificial Sequence: Synthetic peptide
HDNFPL

SEQ ID NO: 518 - protein, 6 aa, synthetic construct
  REGION 1..6: note = Description of Artificial Sequence: Synthetic peptide
YYRFPY

SEQ ID NO: 519 - protein, 8 aa, synthetic construct
  REGION 1..8: note = Description of Artificial Sequence: Synthetic peptide
WDSDSEHV

SEQ ID NO: 520 - protein, 6 aa, synthetic construct
  REGION 1..6: note = Description of Artificial Sequence: Synthetic peptide
WDDTTV

SEQ ID NO: 521 - protein, 6 aa, synthetic construct
  REGION 1..6: note = Description of Artificial Sequence: Synthetic peptide
YNSAPF

SEQ ID NO: 522 - protein, 9 aa, synthetic construct
  REGION 1..9: note = Description of Artificial Sequence: Synthetic peptide
RDSSGDHLR

SEQ ID NO: 523 - protein, 8 aa, synthetic construct
  REGION 1..8: note = Description of Artificial Sequence: Synthetic peptide
RDSSGHHL

SEQ ID NO: 524 - protein, 11 aa, synthetic construct
  REGION 1..11: note = Description of Artificial Sequence: Synthetic peptide
YGSSFNGSSLF

SEQ ID NO: 525 - protein, 7 aa, synthetic construct
  REGION 1..7: note = Description of Artificial Sequence: Synthetic peptide
FGTSSGL

SEQ ID NO: 526 - protein, 7 aa, synthetic construct
  REGION 1..7: note = Description of Artificial Sequence: Synthetic peptide
YESSPSW

SEQ ID NO: 527 - protein, 7 aa, synthetic construct
  REGION 1..7: note = Description of Artificial Sequence: Synthetic peptide
YGSPPRF

SEQ ID NO: 528 - protein, 7 aa, synthetic construct
  REGION 1..7: note = Description of Artificial Sequence: Synthetic peptide
YGSSPSW

SEQ ID NO: 529 - protein, 7 aa, synthetic construct
  REGION 1..7: note = Description of Artificial Sequence: Synthetic peptide
YDSSPSW

SEQ ID NO: 530 - protein, 7 aa, synthetic construct
  REGION 1..7: note = Description of Artificial Sequence: Synthetic peptide
YGTSPMY

SEQ ID NO: 531 - protein, 6 aa, synthetic construct
  REGION 1..6: note = Description of Artificial Sequence: Synthetic peptide
ALQTPL

SEQ ID NO: 532 - protein, 8 aa, synthetic construct
  REGION 1..8: note = Description of Artificial Sequence: Synthetic peptide
YGNSPPKF

SEQ ID NO: 533 - protein, 7 aa, synthetic construct
  REGION 1..7: note = Description of Artificial Sequence: Synthetic peptide
YGGSPRL

SEQ ID NO: 534 - protein, 6 aa, synthetic construct
  REGION 1..6: note = Description of Artificial Sequence: Synthetic peptide
HYSTPW

SEQ ID NO: 535 - protein, 6 aa, synthetic construct
  REGION 1..6: note = Description of Artificial Sequence: Synthetic peptide
SRIFPR

SEQ ID NO: 536 - protein, 6 aa, synthetic construct
  REGION 1..6: note = Description of Artificial Sequence: Synthetic peptide
SRTIPR

SEQ ID NO: 537 - protein, 5 aa, synthetic construct
  REGION 1..5: note = Description of Artificial Sequence: Synthetic peptide
NHGMS

SEQ ID NO: 538 - protein, 5 aa, synthetic construct
  REGION 1..5: note = Description of Artificial Sequence: Synthetic peptide
NYAMS

SEQ ID NO: 539 - protein, 5 aa, synthetic construct
  REGION 1..5: note = Description of Artificial Sequence: Synthetic peptide
DYAMH

SEQ ID NO: 540 - protein, 5 aa, synthetic construct
  REGION 1..5: note = Description of Artificial Sequence: Synthetic peptide
NFGIN

SEQ ID NO: 541 - protein, 5 aa, synthetic construct
  REGION 1..5: note = Description of Artificial Sequence: Synthetic peptide
SDAMT

SEQ ID NO: 542 - protein, 5 aa, synthetic construct
  REGION 1..5: note = Description of Artificial Sequence: Synthetic peptide
NYGIT

SEQ ID NO: 543 - protein, 5 aa, synthetic construct
  REGION 1..5: note = Description of Artificial Sequence: Synthetic peptide
DYYMS

SEQ ID NO: 544 - protein, 7 aa, synthetic construct
  REGION 1..7: note = Description of Artificial Sequence: Synthetic peptide
SSYYYWG

SEQ ID NO: 545 - protein, 7 aa, synthetic construct
  REGION 1..7: note = Description of Artificial Sequence: Synthetic peptide
TSGMCVS

SEQ ID NO: 546 - protein, 5 aa, synthetic construct
  REGION 1..5: note = Description of Artificial Sequence: Synthetic peptide
SYSMN

SEQ ID NO: 547 - protein, 5 aa, synthetic construct
  REGION 1..5: note = Description of Artificial Sequence: Synthetic peptide
SHYIH

SEQ ID NO: 548 - protein, 7 aa, synthetic construct
  REGION 1..7: note = Description of Artificial Sequence: Synthetic peptide
SGGYYWS

SEQ ID NO: 549 - protein, 5 aa, synthetic construct
  REGION 1..5: note = Description of Artificial Sequence: Synthetic peptide
SYAIS

SEQ ID NO: 550 - protein, 7 aa, synthetic construct
  REGION 1..7: note = Description of Artificial Sequence: Synthetic peptide
SNSAAWN

SEQ ID NO: 551 - protein, 5 aa, synthetic construct
  REGION 1..5: note = Description of Artificial Sequence: Synthetic peptide
SYAMS

SEQ ID NO: 552 - protein, 5 aa, synthetic construct
  REGION 1..5: note = Description of Artificial Sequence: Synthetic peptide
RYPMS

SEQ ID NO: 553 - protein, 5 aa, synthetic construct
  REGION 1..5: note = Description of Artificial Sequence: Synthetic peptide
NFGMN

SEQ ID NO: 554 - protein, 5 aa, synthetic construct
  REGION 1..5: note = Description of Artificial Sequence: Synthetic peptide
DYSIN

SEQ ID NO: 555 - protein, 5 aa, synthetic construct
  REGION 1..5: note = Description of Artificial Sequence: Synthetic peptide
HYSMN

SEQ ID NO: 556 - protein, 16 aa, synthetic construct
  REGION 1..16: note = Description of Artificial Sequence: Synthetic peptide
GIVYSGSTYYAASVKG

SEQ ID NO: 557 - protein, 17 aa, synthetic construct
  REGION 1..17: note = Description of Artificial Sequence: Synthetic peptide
GISRSGENTYYADSVKG

SEQ ID NO: 558 - protein, 17 aa, synthetic construct
  REGION 1..17: note = Description of Artificial Sequence: Synthetic peptide
GISWNSGSIGYADSVKG

SEQ ID NO: 559 - protein, 17 aa, synthetic construct
  REGION 1..17: note = Description of Artificial Sequence: Synthetic peptide
WINPKNNNTNYAQKFQG

SEQ ID NO: 560 - protein, 17 aa, synthetic construct
  REGION 1..17: note = Description of Artificial Sequence: Synthetic peptide
VISGSGGTTYYADSVKG

SEQ ID NO: 561 - protein, 17 aa, synthetic construct
  REGION 1..17: note = Description of Artificial Sequence: Synthetic peptide
WISAYNGNTNYAQKFQG

SEQ ID NO: 562 - protein, 17 aa, synthetic construct
  REGION 1..17: note = Description of Artificial Sequence: Synthetic peptide
YISSSGSTIYYADSVKG

SEQ ID NO: 563 - protein, 17 aa, synthetic construct
  REGION 1..17: note = Description of Artificial Sequence: Synthetic peptide
YISSSGNTIYYADSVKG

SEQ ID NO: 564 - protein, 16 aa, synthetic construct
  REGION 1..16: note = Description of Artificial Sequence: Synthetic peptide
SIYYSGSAYYNPSLKS

SEQ ID NO: 565 - protein, 16 aa, synthetic construct
  REGION 1..16: note = Description of Artificial Sequence: Synthetic peptide
RIDWDEDKFYSTSLKT

SEQ ID NO: 566 - protein, 17 aa, synthetic construct
  REGION 1..17: note = Description of Artificial Sequence: Synthetic peptide
SISSSSSYIYYADSVKG

SEQ ID NO: 567 - protein, 17 aa, synthetic construct
  REGION 1..17: note = Description of Artificial Sequence: Synthetic peptide
MINPSGGVTAYSQTLQG

SEQ ID NO: 568 - protein, 16 aa, synthetic construct
  REGION 1..16: note = Description of Artificial Sequence: Synthetic peptide
YIYYSGSTYYNPSLKS

SEQ ID NO: 569 - protein, 17 aa, synthetic construct
  REGION 1..17: note = Description of Artificial Sequence: Synthetic peptide
GIIPIFGTANYAQKFQG

SEQ ID NO: 570 - protein, 18 aa, synthetic construct
  REGION 1..18: note = Description of Artificial Sequence: Synthetic peptide
RTYYRSKWYSFYAISLKS

SEQ ID NO: 571 - protein, 17 aa, synthetic construct
  REGION 1..17: note = Description of Artificial Sequence: Synthetic peptide
AISGSGGSTYYADSVKG

SEQ ID NO: 572 - protein, 17 aa, synthetic construct
  REGION 1..17: note = Description of Artificial Sequence: Synthetic peptide
GISDSGVSTYYADSAKG

SEQ ID NO: 573 - protein, 17 aa, synthetic construct
  REGION 1..17: note = Description of Artificial Sequence: Synthetic peptide
SINWKGNSLAYGDSVKG

SEQ ID NO: 574 - protein, 17 aa, synthetic construct
  REGION 1..17: note = Description of Artificial Sequence: Synthetic peptide
WINTYTGESYFADDFKG

SEQ ID NO: 575 - protein, 17 aa, synthetic construct
  REGION 1..17: note = Description of Artificial Sequence: Synthetic peptide
WINTETREPAYAYDFRG

SEQ ID NO: 576 - protein, 17 aa, synthetic construct
  REGION 1..17: note = Description of Artificial Sequence: Synthetic peptide
RINTESGVPIYADDFKG

SEQ ID NO: 577 - protein, 17 aa, synthetic construct
  REGION 1..17: note = Description of Artificial Sequence: Synthetic peptide
RINTETGEPLYADDFKG

SEQ ID NO: 578 - protein, 7 aa, synthetic construct
  REGION 1..7: note = Description of Artificial Sequence: Synthetic peptide
HGGESDV

SEQ ID NO: 579 - protein, 11 aa, synthetic construct
  REGION 1..11: note = Description of Artificial Sequence: Synthetic peptide
SPAHYYGGMDV

SEQ ID NO: 580 - protein, 6 aa, synthetic construct
  REGION 1..6: note = Description of Artificial Sequence: Synthetic peptide
HSFLAY

SEQ ID NO: 581 - protein, 11 aa, synthetic construct
  REGION 1..11: note = Description of Artificial Sequence: Synthetic peptide
GPYYYQSYMDV

SEQ ID NO: 582 - protein, 14 aa, synthetic construct
  REGION 1..14: note = Description of Artificial Sequence: Synthetic peptide
LDSSGYYYARGPRY

SEQ ID NO: 583 - protein, 9 aa, synthetic construct
  REGION 1..9: note = Description of Artificial Sequence: Synthetic peptide
GPYYYYMDV

SEQ ID NO: 584 - protein, 8 aa, synthetic construct
  REGION 1..8: note = Description of Artificial Sequence: Synthetic peptide
ESGDGMDV

SEQ ID NO: 585 - protein, 8 aa, synthetic construct
  REGION 1..8: note = Description of Artificial Sequence: Synthetic peptide
STMVREDY

SEQ ID NO: 586 - protein, 11 aa, synthetic construct
  REGION 1..11: note = Description of Artificial Sequence: Synthetic peptide
HWQEWPDAFDI

SEQ ID NO: 587 - protein, 13 aa, synthetic construct
  REGION 1..13: note = Description of Artificial Sequence: Synthetic peptide
SGAGGTSATAFDI

SEQ ID NO: 588 - protein, 10 aa, synthetic construct
  REGION 1..10: note = Description of Artificial Sequence: Synthetic peptide
TIAAVYAFDI

SEQ ID NO: 589 - protein, 8 aa, synthetic construct
  REGION 1..8: note = Description of Artificial Sequence: Synthetic peptide
DLRGAFDI

SEQ ID NO: 590 - protein, 11 aa, synthetic construct
  REGION 1..11: note = Description of Artificial Sequence: Synthetic peptide
EGSGSGWYFDF

SEQ ID NO: 591 - protein, 13 aa, synthetic construct
  REGION 1..13: note = Description of Artificial Sequence: Synthetic peptide
AGIAARLRGAFDI

SEQ ID NO: 592 - protein, 20 aa, synthetic construct
  REGION 1..20: note = Description of Artificial Sequence: Synthetic peptide
RGGYQLLRWDVGLLRSAFDI

SEQ ID NO: 593 - protein, 13 aa, synthetic construct
  REGION 1..13: note = Description of Artificial Sequence: Synthetic peptide
SSPEGLFLYWFDP

SEQ ID NO: 594 - protein, 9 aa, synthetic construct
  REGION 1..9: note = Description of Artificial Sequence: Synthetic peptide
VEGSGSLDY

SEQ ID NO: 595 - protein, 9 aa, synthetic construct
  REGION 1..9: note = Description of Artificial Sequence: Synthetic peptide
RAGSEASDI

SEQ ID NO: 596 - protein, 15 aa, synthetic construct
  REGION 1..15: note = Description of Artificial Sequence: Synthetic peptide
ATYKRELRYYYGMDV

SEQ ID NO: 597 - protein, 8 aa, synthetic construct
  REGION 1..8: note = Description of Artificial Sequence: Synthetic peptide
VGKAVPDV

SEQ ID NO: 598 - protein, 13 aa, synthetic construct
  REGION 1..13: note = Description of Artificial Sequence: Synthetic peptide
HQGVAYYNYAMDV

SEQ ID NO: 599 - protein, 8 aa, synthetic construct
  REGION 1..8: note = Description of Artificial Sequence: Synthetic peptide
VVRDGMDV

SEQ ID NO: 600 - protein, 9 aa, synthetic construct
  REGION 1..9: note = Description of Artificial Sequence: Synthetic peptide
IPQTGTFDY

SEQ ID NO: 601 - protein, 15 aa, synthetic construct
  REGION 1..15: note = Description of Artificial Sequence: Synthetic peptide
ANYKRELRYYYGMDV

SEQ ID NO: 602 - protein, 11 aa, synthetic construct
  REGION 1..11: note = Description of Artificial Sequence: Synthetic peptide
ALVGATGAFDI

SEQ ID NO: 603 - protein, 8 aa, synthetic construct
  REGION 1..8: note = Description of Artificial Sequence: Synthetic peptide
WFGEGFDP

SEQ ID NO: 604 - protein, 17 aa, synthetic construct
  REGION 1..17: note = Description of Artificial Sequence: Synthetic peptide
VGYDSSGYYRDYYGMDV

SEQ ID NO: 605 - protein, 13 aa, synthetic construct
  REGION 1..13: note = Description of Artificial Sequence: Synthetic peptide
MGWSSGYLGAFDI

SEQ ID NO: 606 - protein, 13 aa, synthetic construct
  REGION 1..13: note = Description of Artificial Sequence: Synthetic peptide
GEIYYGYDGGFAY

SEQ ID NO: 607 - protein, 8 aa, synthetic construct
  REGION 1..8: note = Description of Artificial Sequence: Synthetic peptide
DYSYAMDY

SEQ ID NO: 608 - protein, 8 aa, synthetic construct
  REGION 1..8: note = Description of Artificial Sequence: Synthetic peptide
DYLYSLDF

SEQ ID NO: 609 - protein, 8 aa, synthetic construct
  REGION 1..8: note = Description of Artificial Sequence: Synthetic peptide
DYLYSCDY

SEQ ID NO: 610 - protein, 7 aa, synthetic construct
  REGION 1..7: note = Description of Artificial Sequence: Synthetic peptide
GFALSNH

SEQ ID NO: 611 - protein, 7 aa, synthetic construct
  REGION 1..7: note = Description of Artificial Sequence: Synthetic peptide
GFTFSNY

SEQ ID NO: 612 - protein, 7 aa, synthetic construct
  REGION 1..7: note = Description of Artificial Sequence: Synthetic peptide
GFTFDDY

SEQ ID NO: 613 - protein, 7 aa, synthetic construct
  REGION 1..7: note = Description of Artificial Sequence: Synthetic peptide
GYIFDNF

SEQ ID NO: 614 - protein, 7 aa, synthetic construct
  REGION 1..7: note = Description of Artificial Sequence: Synthetic peptide
GFTFSSD

SEQ ID NO: 615 - protein, 7 aa, synthetic construct
  REGION 1..7: note = Description of Artificial Sequence: Synthetic peptide
GYTFSNY

SEQ ID NO: 616 - protein, 7 aa, synthetic construct
  REGION 1..7: note = Description of Artificial Sequence: Synthetic peptide
GFTFSDY

SEQ ID NO: 617 - protein, 9 aa, synthetic construct
  REGION 1..9: note = Description of Artificial Sequence: Synthetic peptide
GGSISSSYY

SEQ ID NO: 618 - protein, 9 aa, synthetic construct
  REGION 1..9: note = Description of Artificial Sequence: Synthetic peptide
GFSLRTSGM

SEQ ID NO: 619 - protein, 7 aa, synthetic construct
  REGION 1..7: note = Description of Artificial Sequence: Synthetic peptide
GFTFSSY

SEQ ID NO: 620 - protein, 7 aa, synthetic construct
  REGION 1..7: note = Description of Artificial Sequence: Synthetic peptide
GYTVTSH

SEQ ID NO: 621 - protein, 9 aa, synthetic construct
  REGION 1..9: note = Description of Artificial Sequence: Synthetic peptide
GGSISSGGY

SEQ ID NO: 622 - protein, 7 aa, synthetic construct
  REGION 1..7: note = Description of Artificial Sequence: Synthetic peptide
GGTFSSY

SEQ ID NO: 623 - protein, 9 aa, synthetic construct
  REGION 1..9: note = Description of Artificial Sequence: Synthetic peptide
GDSVSSNSA

SEQ ID NO: 624 - protein, 7 aa, synthetic construct
  REGION 1..7: note = Description of Artificial Sequence: Synthetic peptide
GITFSRY

SEQ ID NO: 625 - protein, 7 aa, synthetic construct
  REGION 1..7: note = Description of Artificial Sequence: Synthetic peptide
GFSFSSY

SEQ ID NO: 626 - protein, 7 aa, synthetic construct
  REGION 1..7: note = Description of Artificial Sequence: Synthetic peptide
GYTFTNF

SEQ ID NO: 627 - protein, 7 aa, synthetic construct
  REGION 1..7: note = Description of Artificial Sequence: Synthetic peptide
GYTFTDY

SEQ ID NO: 628 - protein, 7 aa, synthetic construct
  REGION 1..7: note = Description of Artificial Sequence: Synthetic peptide
GYTFRHY

SEQ ID NO: 629 - protein, 7 aa, synthetic construct
  REGION 1..7: note = Description of Artificial Sequence: Synthetic peptide
GYTFTHY

SEQ ID NO: 630 - protein, 5 aa, synthetic construct
  REGION 1..5: note = Description of Artificial Sequence: Synthetic peptide
VYSGS

SEQ ID NO: 631 - protein, 6 aa, synthetic construct
  REGION 1..6: note = Description of Artificial Sequence: Synthetic peptide
SRSGEN

SEQ ID NO: 632 - protein, 6 aa, synthetic construct
  REGION 1..6: note = Description of Artificial Sequence: Synthetic peptide
SWNSGS

SEQ ID NO: 633 - protein, 6 aa, synthetic construct
  REGION 1..6: note = Description of Artificial Sequence: Synthetic peptide
NPKNNN

SEQ ID NO: 634 - protein, 6 aa, synthetic construct
  REGION 1..6: note = Description of Artificial Sequence: Synthetic peptide
SGSGGT

SEQ ID NO: 635 - protein, 6 aa, synthetic construct
  REGION 1..6: note = Description of Artificial Sequence: Synthetic peptide
SAYNGN

SEQ ID NO: 636 - protein, 6 aa, synthetic construct
  REGION 1..6: note = Description of Artificial Sequence: Synthetic peptide
SSSGST

SEQ ID NO: 637 - protein, 6 aa, synthetic construct
  REGION 1..6: note = Description of Artificial Sequence: Synthetic peptide
SSSGNT

SEQ ID NO: 638 - protein, 5 aa, synthetic construct
  REGION 1..5: note = Description of Artificial Sequence: Synthetic peptide
YYSGS

SEQ ID NO: 639 - protein, 5 aa, synthetic construct
  REGION 1..5: note = Description of Artificial Sequence: Synthetic peptide
DWDED

SEQ ID NO: 640 - protein, 6 aa, synthetic construct
  REGION 1..6: note = Description of Artificial Sequence: Synthetic peptide
SSSSSY

SEQ ID NO: 641 - protein, 6 aa, synthetic construct
  REGION 1..6: note = Description of Artificial Sequence: Synthetic peptide
NPSGGV

SEQ ID NO: 642 - protein, 6 aa, synthetic construct
  REGION 1..6: note = Description of Artificial Sequence: Synthetic peptide
IPIFGT

SEQ ID NO: 643 - protein, 7 aa, synthetic construct
  REGION 1..7: note = Description of Artificial Sequence: Synthetic peptide
YYRSKWY

SEQ ID NO: 644 - protein, 6 aa, synthetic construct
  REGION 1..6: note = Description of Artificial Sequence: Synthetic peptide
SGSGGS

SEQ ID NO: 645 - protein, 6 aa, synthetic construct
  REGION 1..6: note = Description of Artificial Sequence: Synthetic peptide
SDSGVS

SEQ ID NO: 646 - protein, 6 aa, synthetic construct
  REGION 1..6: note = Description of Artificial Sequence: Synthetic peptide
NWKGNS

SEQ ID NO: 647 - protein, 6 aa, synthetic construct
  REGION 1..6: note = Description of Artificial Sequence: Synthetic peptide
NTYTGE

SEQ ID NO: 648 - protein, 6 aa, synthetic construct
  REGION 1..6: note = Description of Artificial Sequence: Synthetic peptide
NTETRE

SEQ ID NO: 649 - protein, 6 aa, synthetic construct
  REGION 1..6: note = Description of Artificial Sequence: Synthetic peptide
NTESGV

SEQ ID NO: 650 - protein, 6 aa, synthetic construct
  REGION 1..6: note = Description of Artificial Sequence: Synthetic peptide
NTETGE

SEQ ID NO: 651 - protein, 10 aa, synthetic construct
  REGION 1..10: note = Description of Artificial Sequence: Synthetic peptide
GFALSNHGMS

SEQ ID NO: 652 - protein, 10 aa, synthetic construct
  REGION 1..10: note = Description of Artificial Sequence: Synthetic peptide
GFTFSNYAMS

SEQ ID NO: 653 - protein, 10 aa, synthetic construct
  REGION 1..10: note = Description of Artificial Sequence: Synthetic peptide
GFTFDDYAMH

SEQ ID NO: 654 - protein, 10 aa, synthetic construct
  REGION 1..10: note = Description of Artificial Sequence: Synthetic peptide
GYIFDNFGIN

SEQ ID NO: 655 - protein, 10 aa, synthetic construct
  REGION 1..10: note = Description of Artificial Sequence: Synthetic peptide
GFTFSSDAMT

SEQ ID NO: 656 - protein, 10 aa, synthetic construct
  REGION 1..10: note = Description of Artificial Sequence: Synthetic peptide
GYTFSNYGIT

SEQ ID NO: 657 - protein, 10 aa, synthetic construct
  REGION 1..10: note = Description of Artificial Sequence: Synthetic peptide
GFTFSDYYMS

SEQ ID NO: 658 - protein, 12 aa, synthetic construct
  REGION 1..12: note = Description of Artificial Sequence: Synthetic peptide
GGSISSSYYYWG

SEQ ID NO: 659 - protein, 12 aa, synthetic construct
  REGION 1..12: note = Description of Artificial Sequence: Synthetic peptide
GFSLRTSGMCVS

SEQ ID NO: 660 - protein, 10 aa, synthetic construct
  REGION 1..10: note = Description of Artificial Sequence: Synthetic peptide
GFTFSSYSMN

SEQ ID NO: 661 - protein, 10 aa, synthetic construct
  REGION 1..10: note = Description of Artificial Sequence: Synthetic peptide
GYTVTSHYIH

SEQ ID NO: 662 - protein, 12 aa, synthetic construct
  REGION 1..12: note = Description of Artificial Sequence: Synthetic peptide
GGSISSGGYYWS

SEQ ID NO: 663 - protein, 10 aa, synthetic construct
  REGION 1..10: note = Description of Artificial Sequence: Synthetic peptide
GGTFSSYAIS

SEQ ID NO: 664 - protein, 12 aa, synthetic construct
  REGION 1..12: note = Description of Artificial Sequence: Synthetic peptide
GDSVSSNSAAWN

SEQ ID NO: 665 - protein, 10 aa, synthetic construct
  REGION 1..10: note = Description of Artificial Sequence: Synthetic peptide
GFTFSSYAMS

SEQ ID NO: 666 - protein, 10 aa, synthetic construct
  REGION 1..10: note = Description of Artificial Sequence: Synthetic peptide
GITFSRYPMS

SEQ ID NO: 667 - protein, 10 aa, synthetic construct
  REGION 1..10: note = Description of Artificial Sequence: Synthetic peptide
GFSFSSYAMS

SEQ ID NO: 668 - protein, 10 aa, synthetic construct
  REGION 1..10: note = Description of Artificial Sequence: Synthetic peptide
GYTFTNFGMN

SEQ ID NO: 669 - protein, 10 aa, synthetic construct
  REGION 1..10: note = Description of Artificial Sequence: Synthetic peptide
GYTFTDYSIN

SEQ ID NO: 670 - protein, 10 aa, synthetic construct
  REGION 1..10: note = Description of Artificial Sequence: Synthetic peptide
GYTFRHYSMN

SEQ ID NO: 671 - protein, 10 aa, synthetic construct
  REGION 1..10: note = Description of Artificial Sequence: Synthetic peptide
GYTFTHYSMN

SEQ ID NO: 672 - protein, 5 aa, synthetic construct
  REGION 1..5: note = Description of Artificial Sequence: Synthetic peptide
DYGVS

SEQ ID NO: 673 - protein, 16 aa, synthetic construct
  REGION 1..16: note = Description of Artificial Sequence: Synthetic peptide
VIWGSETTYYNSALKS

SEQ ID NO: 674 - protein, 16 aa, synthetic construct
  REGION 1..16: note = Description of Artificial Sequence: Synthetic peptide
VIWGSETTYYSSSLKS

SEQ ID NO: 675 - protein, 16 aa, synthetic construct
  REGION 1..16: note = Description of Artificial Sequence: Synthetic peptide
VIWGSETTYYQSSLKS

SEQ ID NO: 676 - protein, 16 aa, synthetic construct
  REGION 1..16: note = Description of Artificial Sequence: Synthetic peptide
VIWGSETTYYNSSLKS

SEQ ID NO: 677 - protein, 12 aa, synthetic construct
  REGION 1..12: note = Description of Artificial Sequence: Synthetic peptide
HYYYGGSYAMDY

SEQ ID NO: 678 - protein, 11 aa, synthetic construct
  REGION 1..11: note = Description of Artificial Sequence: Synthetic peptide
RASQDISKYLN

SEQ ID NO: 679 - protein, 7 aa, synthetic construct
  REGION 1..7: note = Description of Artificial Sequence: Synthetic peptide
HTSRLHS

SEQ ID NO: 680 - protein, 9 aa, synthetic construct
  REGION 1..9: note = Description of Artificial Sequence: Synthetic peptide
QQGNTLPYT

SEQ ID NO: 681 - protein, 120 aa, synthetic construct
  REGION 1..120: note = Description of Artificial Sequence: Synthetic polypeptide
EVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSS

SEQ ID NO: 682 - protein, 120 aa, synthetic construct
  REGION 1..120: note = Description of Artificial Sequence: Synthetic polypeptide
QVQLQESGPGLVKPSETLSLTCTVSGVSLPDYGVSWIRQPPGKGLEWIGVIWGSETTYYSSSLKSRVTISKDNSKNQVSLKLSSVTAADTAVYYCAKHYYYGGSYAMDYWGQGTLVTVSS

SEQ ID NO: 683 - protein, 120 aa, synthetic construct
  REGION 1..120: note = Description of Artificial Sequence: Synthetic polypeptide
QVQLQESGPGLVKPSETLSLTCTVSGVSLPDYGVSWIRQPPGKGLEWIGVIWGSETTYYQSSLKSRVTISKDNSKNQVSLKLSSVTAADTAVYYCAKHYYYGGSYAMDYWGQGTLVTVSS

SEQ ID NO: 684 - protein, 120 aa, synthetic construct
  REGION 1..120: note = Description of Artificial Sequence: Synthetic polypeptide
QVQLQESGPGLVKPSETLSLTCTVSGVSLPDYGVSWIRQPPGKGLEWIGVIWGSETTYYNSSLKSRVTISKDNSKNQVSLKLSSVTAADTAVYYCAKHYYYGGSYAMDYWGQGTLVTVSS

SEQ ID NO: 685 - protein, 107 aa, synthetic construct
  REGION 1..107: note = Description of Artificial Sequence: Synthetic polypeptide
DIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEIT

SEQ ID NO: 686 - protein, 107 aa, synthetic construct
  REGION 1..107: note = Description of Artificial Sequence: Synthetic polypeptide
EIVMTQSPATLSLSPGERATLSCRASQDISKYLNWYQQKPGQAPRLLIYHTSRLHSGIPARFSGSGSGTDYTLTISSLQPEDFAVYFCQQGNTLPYTFGQGTKLEIK

SEQ ID NO: 687 - protein, 242 aa, synthetic construct
  REGION 1..242: note = Description of Artificial Sequence: Synthetic polypeptide
EIVMTQSPATLSLSPGERATLSCRASQDISKYLNWYQQKPGQAPRLLIYHTSRLHSGIPARFSGSGSGTDYTLTISSLQPEDFAVYFCQQGNTLPYTFGQGTKLEIKGGGGSGGGGSGGGGSQVQLQESGPGLVKPSETLSLTCTVSGVSLPDYGVSWIRQPPGKGLEWIGVIWGSETTYYSSSLKSRVTISKDNSKNQVSLKLSSVTAADTAVYYCAKHYYYGGSYAMDYWGQGTLVTVSS

SEQ ID NO: 688 - protein, 242 aa, synthetic construct
  REGION 1..242: note = Description of Artificial Sequence: Synthetic polypeptide
EIVMTQSPATLSLSPGERATLSCRASQDISKYLNWYQQKPGQAPRLLIYHTSRLHSGIPARFSGSGSGTDYTLTISSLQPEDFAVYFCQQGNTLPYTFGQGTKLEIKGGGGSGGGGSGGGGSQVQLQESGPGLVKPSETLSLTCTVSGVSLPDYGVSWIRQPPGKGLEWIGVIWGSETTYYQSSLKSRVTISKDNSKNQVSLKLSSVTAADTAVYYCAKHYYYGGSYAMDYWGQGTLVTVSS

SEQ ID NO: 689 - protein, 242 aa, synthetic construct
  REGION 1..242: note = Description of Artificial Sequence: Synthetic polypeptide
QVQLQESGPGLVKPSETLSLTCTVSGVSLPDYGVSWIRQPPGKGLEWIGVIWGSETTYYSSSLKSRVTISKDNSKNQVSLKLSSVTAADTAVYYCAKHYYYGGSYAMDYWGQGTLVTVSSGGGGSGGGGSGGGGSEIVMTQSPATLSLSPGERATLSCRASQDISKYLNWYQQKPGQAPRLLIYHTSRLHSGIPARFSGSGSGTDYTLTISSLQPEDFAVYFCQQGNTLPYTFGQGTKLEIK

SEQ ID NO: 690 - protein, 242 aa, synthetic construct
  REGION 1..242: note = Description of Artificial Sequence: Synthetic polypeptide
QVQLQESGPGLVKPSETLSLTCTVSGVSLPDYGVSWIRQPPGKGLEWIGVIWGSETTYYQSSLKSRVTISKDNSKNQVSLKLSSVTAADTAVYYCAKHYYYGGSYAMDYWGQGTLVTVSSGGGGSGGGGSGGGGSEIVMTQSPATLSLSPGERATLSCRASQDISKYLNWYQQKPGQAPRLLIYHTSRLHSGIPARFSGSGSGTDYTLTISSLQPEDFAVYFCQQGNTLPYTFGQGTKLEIK

SEQ ID NO: 691 - protein, 247 aa, synthetic construct
  REGION 1..247: note = Description of Artificial Sequence: Synthetic polypeptide
EIVMTQSPATLSLSPGERATLSCRASQDISKYLNWYQQKPGQAPRLLIYHTSRLHSGIPARFSGSGSGTDYTLTISSLQPEDFAVYFCQQGNTLPYTFGQGTKLEIKGGGGSGGGGSGGGGSGGGGSQVQLQESGPGLVKPSETLSLTCTVSGVSLPDYGVSWIRQPPGKGLEWIGVIWGSETTYYSSSLKSRVTISKDNSKNQVSLKLSSVTAADTAVYYCAKHYYYGGSYAMDYWGQGTLVTVSS

SEQ ID NO: 692 - protein, 247 aa, synthetic construct
  REGION 1..247: note = Description of Artificial Sequence: Synthetic polypeptide
EIVMTQSPATLSLSPGERATLSCRASQDISKYLNWYQQKPGQAPRLLIYHTSRLHSGIPARFSGSGSGTDYTLTISSLQPEDFAVYFCQQGNTLPYTFGQGTKLEIKGGGGSGGGGSGGGGSGGGGSQVQLQESGPGLVKPSETLSLTCTVSGVSLPDYGVSWIRQPPGKGLEWIGVIWGSETTYYQSSLKSRVTISKDNSKNQVSLKLSSVTAADTAVYYCAKHYYYGGSYAMDYWGQGTLVTVSS

SEQ ID NO: 693 - protein, 247 aa, synthetic construct
  REGION 1..247: note = Description of Artificial Sequence: Synthetic polypeptide
QVQLQESGPGLVKPSETLSLTCTVSGVSLPDYGVSWIRQPPGKGLEWIGVIWGSETTYYSSSLKSRVTISKDNSKNQVSLKLSSVTAADTAVYYCAKHYYYGGSYAMDYWGQGTLVTVSSGGGGSGGGGSGGGGSGGGGSEIVMTQSPATLSLSPGERATLSCRASQDISKYLNWYQQKPGQAPRLLIYHTSRLHSGIPARFSGSGSGTDYTLTISSLQPEDFAVYFCQQGNTLPYTFGQGTKLEIK

SEQ ID NO: 694 - protein, 247 aa, synthetic construct
  REGION 1..247: note = Description of Artificial Sequence: Synthetic polypeptide
QVQLQESGPGLVKPSETLSLTCTVSGVSLPDYGVSWIRQPPGKGLEWIGVIWGSETTYYQSSLKSRVTISKDNSKNQVSLKLSSVTAADTAVYYCAKHYYYGGSYAMDYWGQGTLVTVSSGGGGSGGGGSGGGGSGGGGSEIVMTQSPATLSLSPGERATLSCRASQDISKYLNWYQQKPGQAPRLLIYHTSRLHSGIPARFSGSGSGTDYTLTISSLQPEDFAVYFCQQGNTLPYTFGQGTKLEIK

SEQ ID NO: 695 - protein, 247 aa, synthetic construct
  REGION 1..247: note = Description of Artificial Sequence: Synthetic polypeptide
EIVMTQSPATLSLSPGERATLSCRASQDISKYLNWYQQKPGQAPRLLIYHTSRLHSGIPARFSGSGSGTDYTLTISSLQPEDFAVYFCQQGNTLPYTFGQGTKLEIKGGGGSGGGGSGGGGSGGGGSQVQLQESGPGLVKPSETLSLTCTVSGVSLPDYGVSWIRQPPGKGLEWIGVIWGSETTYYNSSLKSRVTISKDNSKNQVSLKLSSVTAADTAVYYCAKHYYYGGSYAMDYWGQGTLVTVSS

SEQ ID NO: 696 - protein, 247 aa, synthetic construct
  REGION 1..247: note = Description of Artificial Sequence: Synthetic polypeptide
QVQLQESGPGLVKPSETLSLTCTVSGVSLPDYGVSWIRQPPGKGLEWIGVIWGSETTYYNSSLKSRVTISKDNSKNQVSLKLSSVTAADTAVYYCAKHYYYGGSYAMDYWGQGTLVTVSSGGGGSGGGGSGGGGSGGGGSEIVMTQSPATLSLSPGERATLSCRASQDISKYLNWYQQKPGQAPRLLIYHTSRLHSGIPARFSGSGSGTDYTLTISSLQPEDFAVYFCQQGNTLPYTFGQGTKLEIK

SEQ ID NO: 697 - protein, 242 aa, synthetic construct
  REGION 1..242: note = Description of Artificial Sequence: Synthetic polypeptide
EIVMTQSPATLSLSPGERATLSCRASQDISKYLNWYQQKPGQAPRLLIYHTSRLHSGIPARFSGSGSGTDYTLTISSLQPEDFAVYFCQQGNTLPYTFGQGTKLEIKGGGGSGGGGSGGGGSQVQLQESGPGLVKPSETLSLTCTVSGVSLPDYGVSWIRQPPGKGLEWIGVIWGSETTYYNSSLKSRVTISKDNSKNQVSLKLSSVTAADTAVYYCAKHYYYGGSYAMDYWGQGTLVTVSS

SEQ ID NO: 698 - protein, 242 aa, synthetic construct
  REGION 1..242: note = Description of Artificial Sequence: Synthetic polypeptide
QVQLQESGPGLVKPSETLSLTCTVSGVSLPDYGVSWIRQPPGKGLEWIGVIWGSETTYYNSSLKSRVTISKDNSKNQVSLKLSSVTAADTAVYYCAKHYYYGGSYAMDYWGQGTLVTVSSGGGGSGGGGSGGGGSEIVMTQSPATLSLSPGERATLSCRASQDISKYLNWYQQKPGQAPRLLIYHTSRLHSGIPARFSGSGSGTDYTLTISSLQPEDFAVYFCQQGNTLPYTFGQGTKLEIK

SEQ ID NO: 699 - protein, 214 aa, synthetic construct
  REGION 1..214: note = Description of Artificial Sequence: Synthetic polypeptide
EIVMTQSPATLSLSPGERATLSCRASQDISKYLNWYQQKPGQAPRLLIYHTSRLHSGIPARFSGSGSGTDYTLTISSLQPEDFAVYFCQQGNTLPYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 700 - protein, 450 aa, synthetic construct
  REGION 1..450: note = Description of Artificial Sequence: Synthetic polypeptide
QVQLQESGPGLVKPSETLSLTCTVSGVSLPDYGVSWIRQPPGKGLEWIGVIWGSETTYYQSSLKSRVTISKDNSKNQVSLKLSSVTAADTAVYYCAKHYYYGGSYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSRLTVDKSRWQQGNVFSCSVMHEALHNHYTQKSLSLSPGK

SEQ ID NO: 701 - protein, 215 aa, synthetic construct
  REGION 1..215: note = Description of Artificial Sequence: Synthetic polypeptide
QAVVTQEPSLTVSPGGTVTLTCRSSTGAVTTSNYANWVQQKPGQAPRGLIGGTNKRAPWTPARFSGSLLGDKAALTLSGAQPEDEAEYFCALWYSNLWVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 702 - protein, 455 aa, synthetic construct
  REGION 1..455: note = Description of Artificial Sequence: Synthetic polypeptide
EVQLVESGGGLVQPGGSLKLSCAASGFTFNTYAMNWVRQASGKGLEWVGRIRSKYNNYATYYADSVKDRFTISRDDSKSTLYLQMNSLKTEDTAVYYCVRHGNFGNSYVSWFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFLLYSKLTVDKSRWQQGNVFSCSVMHEALHNHYTQKSLSLSPGK

SEQ ID NO: 703 - protein, 213 aa, synthetic construct
  REGION 1..213: note = Description of Artificial Sequence: Synthetic polypeptide
QIVLSQSPAILSASPGEKVTMTCRASSSVSYIHWFQQKPGSSPKPWIYATSNLASGVPVRFSGSGSGTSYSLTISRVEAEDAATYYCQQWTSNPPTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 704 - protein, 451 aa, synthetic construct
  REGION 1..451: note = Description of Artificial Sequence: Synthetic polypeptide
QVQLQQPGAELVKPGASVKMSCKASGYTFTSYNMHWVKQTPGRGLEWIGAIYPGNGDTSYNQKFKGKATLTADKSSSTAYMQLSSLTSEDSAVYYCARSTYYGGDWYFNVWGAGTTVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSRLTVDKSRWQQGNVFSCSVMHEALHNHYTQKSLSLSPGK

SEQ ID NO: 705 - protein, 451 aa, synthetic construct
  REGION 1..451: note = Description of Artificial Sequence: Synthetic polypeptide
QVQLQQPGAELVKPGASVKMSCKASGYTFTSYNMHWVKQTPGRGLEWIGAIYPGNGDTSYNQKFKGKATLTADKSSSTAYMQLSSLTSEDSAVYYCARSTYYGGDWYFNVWGAGTTVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFLLYSKLTVDKSRWQQGNVFSCSVMHEALHNHYTQKSLSLSPGK

SEQ ID NO: 706 - protein, 669 aa, synthetic construct
  REGION 1..669: note = Description of Artificial Sequence: Synthetic polypeptide
EIVMTQSPATLSLSPGERATLSCRASQDISKYLNWYQQKPGQAPRLLIYHTSRLHSGIPARFSGSGSGTDYTLTISSLQPEDFAVYFCQQGNTLPYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECQVQLVESGGGLVQPGGSLKLSCAASGFTFNTYAMNWVRQASGKGLEWVGRIRSKYNNYATYYADSVKDRFTISRDDSKSTLYLQMNSLKTEDTAVYYCVRHGNFGNSYVSWFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSRLTVDKSRWQQGNVFSCSVMHEALHNHYTQKSLSLSPGK

SEQ ID NO: 707 - protein, 223 aa, synthetic construct
  REGION 1..223: note = Description of Artificial Sequence: Synthetic polypeptide
QVQLQESGPGLVKPSETLSLTCTVSGVSLPDYGVSWIRQPPGKGLEWIGVIWGSETTYYQSSLKSRVTISKDNSKNQVSLKLSSVTAADTAVYYCAKHYYYGGSYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSC

SEQ ID NO: 708 - protein, 450 aa, synthetic construct
  REGION 1..450: note = Description of Artificial Sequence: Synthetic polypeptide
QVQLQESGPGLVKPSETLSLTCTVSGVSLPDYGVSWIRQPPGKGLEWIGVIWGSETTYYQSSLKSRVTISKDNSKNQVSLKLSSVTAADTAVYYCAKHYYYGGSYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVCTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 709 - protein, 486 aa, synthetic construct
  REGION 1..486: note = Description of Artificial Sequence: Synthetic polypeptide
EVQLVESGGGLVQPGGSLKLSCAASGFTFNTYAMNWVRQASGKGLEWVGRIRSKYNNYATYYADSVKDRFTISRDDSKSTLYLQMNSLKTEDTAVYYCVRHGNFGNSYVSWFAYWGQGTLVTVSSGGGGSGGGGSGGGGSGGGGSQAVVTQEPSLTVSPGGTVTLTCRSSTGAVTTSNYANWVQQKPGQAPRGLIGGTNKRAPWTPARFSGSLLGDKAALTLSGAQPEDEAEYFCALWYSNLWVFGGGTKLTVLGGGGSDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPCREEMTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 710 - protein, 214 aa, synthetic construct
  REGION 1..214: note = Description of Artificial Sequence: Synthetic polypeptide
DIQMTQSPSSLSASVGDRVTITCSASQDISNYLNWYQQKPGKAPKLLIYYTSNLHSGVPSRFSGSGSGTDFTLTISSLQPEDFATYYCQQYRKLPWTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 711 - protein, 451 aa, synthetic construct
  REGION 1..451: note = Description of Artificial Sequence: Synthetic polypeptide
QVQLVQSGAEVKKPGSSVKVSCKASGGTFSNYWMHWVRQAPGQGLEWMGATYRGHSDTYYNQKFKGRVTITADKSTSTAYMELSSLRSEDTAVYYCARGAIYNGYDVLDNWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVAVSHEDPEVKFNWYVDGVEVHNAKTKPREEQYNSTYRVVSVLTVLHQDWLNGKEYKCKVSNKALAAPIEKTISKAKGQPREPQVCTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 712 - protein, 485 aa, synthetic construct
  REGION 1..485: note = Description of Artificial Sequence: Synthetic polypeptide
EVQLVESGGGLVQPGGSLKLSCAASGFTFNTYAMNWVRQASGKGLEWVGRIRSKYNNYATYYADSVKDRFTISRDDSKSTLYLQMNSLKTEDTAVYYCVRHGNFGNSYVSWFAYWGQGTLVTVSSGGGGSGGGGSGGGGSGGGGSQAVVTQEPSLTVSPGGTVTLTCRSSTGAVTTSNYANWVQQKPGQAPRGLIGGTNKRAPWTPARFSGSLLGDKAALTLSGAQPEDEAEYFCALWYSNLWVFGGGTKLTVLGGGGSDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPCREEMTKQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 713 - protein, 725 aa, synthetic construct
  REGION 1..725: note = Description of Artificial Sequence: Synthetic polypeptide
DIQMTQSPSSLSASVGDRVTITCSASQDISNYLNWYQQKPGKAPKLLIYYTSNLHSGVPSRFSGSGSGTDFTLTISSLQPEDFATYYCQQYRKLPWTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGGGGSGGTGSEVQLVESGGGLVQPGGSLKLSCAASGFTFNTYAMNWVRQASGKGLEWVGRIRSKYNNYATYYADSVKDRFTISRDDSKSTLYLQMNSLKTEDTAVYYCVRHGNFGNSYVSWFAYWGQGTLVTVSSGGGGSGGGGSGGGGSGGGGSQAVVTQEPSLTVSPGGTVTLTCRSSTGAVTTSNYANWVQQKPGQAPRGLIGGTNKRAPWTPARFSGSLLGDKAALTLSGAQPEDEAEYFCALWYSNLWVFGGGTKLTVLSGGGSGGGGSGGGGSGGGGSDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPCREEMTKNQVSLWCLVKGFYPSDIAVEWESNGQPENNYKTTPPVLDSDGSFFLYSKLTVDKSRWQQGNVFSCSVMHEALHNRYTQKSLSLSPGK

SEQ ID NO: 714 - protein, 224 aa, synthetic construct
  REGION 1..224: note = Description of Artificial Sequence: Synthetic polypeptide
QVQLVQSGAEVKKPGSSVKVSCKASGGTFSNYWMHWVRQAPGQGLEWMGATYRGHSDTYYNQKFKGRVTITADKSTSTAYMELSSLRSEDTAVYYCARGAIYNGYDVLDNWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSC

SEQ ID NO: 715 - protein, 674 aa, synthetic construct
  REGION 1..674: note = Description of Artificial Sequence: Synthetic polypeptide
DIQMTQSPSSLSASVGDRVTITCSASQDISNYLNWYQQKPGKAPKLLIYYTSNLHSGVPSRFSGSGSGTDFTLTISSLQPEDFATYYCQQYRKLPWTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGGGGSGGGGSQVQLQESGPGLVKPSETLSLTCTVSGVSLPDYGVSWIRQPPGKGLEWIGVIWGSETTYYQSSLKSRVTISKDNSKNQVSLKLSSVTAADTAVYYCAKHYYYGGSYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVCTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 716 - protein, 486 aa, synthetic construct
  REGION 1..486: note = Description of Artificial Sequence: Synthetic polypeptide
EVQLVESGGGLVQPGGSLKLSCAASGFTFNTYAMNWVRQASGKGLEWVGRIRSKYNNYATYYADSVKDRFTISRDDSKSTLYLQMNSLKTEDTAVYYCVRHGNFGNSYVSWFAYWGQGTLVTVSSGGGGSGGGGSGGGGSGGGGSQAVVTQEPSLTVSPGGTVTLTCRSSTGAVTTSNYANWVQQKPGQAPRGLIGGTNKRAPWTPARFSGSLLGDKAALTLSGAQPEDEAEYFCALWYSNLWVFGGGTKLTVLGGGGSDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPCREEMTKNQVSLWCLVKGFYPSDIAVEWESNGQPENNYKTTPPVLDSDGSFFLYSKLTVDKSRWQQGNVFSCSVMHEALHNRYTQKSLSLSPGK

SEQ ID NO: 717 - protein, 679 aa, synthetic construct
  REGION 1..679: note = Description of Artificial Sequence: Synthetic polypeptide
QVQLQESGPGLVKPSETLSLTCTVSGVSLPDYGVSWIRQPPGKGLEWIGVIWGSETTYYQSSLKSRVTISKDNSKNQVSLKLSSVTAADTAVYYCAKHYYYGGSYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCGGGGSGGGGSDIQMTQSPSSLSASVGDRVTITCSASQDISNYLNWYQQKPGKAPKLLIYYTSNLHSGVPSRFSGSGSGTDFTLTISSLQPEDFATYYCQQYRKLPWTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGGGGSDKTHTCPPCPAPELLGGPSVFLFPPKPKDTLMISRTPEVTCVVVAVSHEDPEVKFNWYVDGVEVHNAKTKPREEQYNSTYRVVSVLTVLHQDWLNGKEYKCKVSNKALAAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 718 - protein, 443 aa, synthetic construct
  REGION 1..443: note = Description of Artificial Sequence: Synthetic polypeptide
DIQMTQSPSSLSASVGDRVTITCSASQDISNYLNWYQQKPGKAPKLLIYYTSNLHSGVPSRFSGSGSGTDFTLTISSLQPEDFATYYCQQYRKLPWTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGSDKTHTCPPCPAPELLGGPSVFLFPPKPKDTLMISRTPEVTCVVVAVSHEDPEVKFNWYVDGVEVHNAKTKPREEQYNSTYRVVSVLTVLHQDWLNGKEYKCKVSNKALAAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 719 - protein, 729 aa, synthetic construct
  REGION 1..729: note = Description of Artificial Sequence: Synthetic polypeptide
EVQLVESGGGLVQPGGSLKLSCAASGFTFNTYAMNWVRQASGKGLEWVGRIRSKYNNYATYYADSVKDRFTISRDDSKSTLYLQMNSLKTEDTAVYYCVRHGNFGNSYVSWFAYWGQGTLVTVSSGGGGSGGGGSGGGGSGGGGSQAVVTQEPSLTVSPGGTVTLTCRSSTGAVTTSNYANWVQQKPGQAPRGLIGGTNKRAPWTPARFSGSLLGDKAALTLSGAQPEDEAEYFCALWYSNLWVFGGGTKLTVLGGGGSDKTHTCPPCPAPELLGGPSVFLFPPKPKDTLMISRTPEVTCVVVAVSHEDPEVKFNWYVDGVEVHNAKTKPREEQYNSTYRVVSVLTVLHQDWLNGKEYKCKVSNKALAAPIEKTISKAKGQPREPQVYTLPPCREEMTKNQVSLWCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSGGGGSQVQLQESGPGLVKPSETLSLTCTVSGVSLPDYGVSWIRQPPGKGLEWIGVIWGSETTYYQSSLKSRVTISKDNSKNQVSLKLSSVTAADTAVYYCAKHYYYGGSYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSC

SEQ ID NO: 720 - protein, 720 aa, synthetic construct
  REGION 1..720: note = Description of Artificial Sequence: Synthetic polypeptide
EVQLVESGGGLVQPGGSLKLSCAASGFTFNTYAMNWVRQASGKGLEWVGRIRSKYNNYATYYADSVKDRFTISRDDSKSTLYLQMNSLKTEDTAVYYCVRHGNFGNSYVSWFAYWGQGTLVTVSSGGGGSGGGGSGGGGSGGGGSQAVVTQEPSLTVSPGGTVTLTCRSSTGAVTTSNYANWVQQKPGQAPRGLIGGTNKRAPWTPARFSGSLLGDKAALTLSGAQPEDEAEYFCALWYSNLWVFGGGTKLTVLGGGGSDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPCREEMTKNQVSLWCLVKGFYPSDIAVEWESNGQPENNYKTTPPVLDSDGSFFLYSKLTVDKSRWQQGNVFSCSVMHEALHNRYTQKSLSLSPGKGGGGSGGGGSGGGGSGGGGSDIQMTQSPSSLSASVGDRVTITCSASQDISNYLNWYQQKPGKAPKLLIYYTSNLHSGVPSRFSGSGSGTDFTLTISSLQPEDFATYYCQQYRKLPWTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 721 - protein, 753 aa, synthetic construct
  REGION 1..753: note = Description of Artificial Sequence: Synthetic polypeptide
EVQLVESGGGLVQPGGSLKLSCAASGFTFNTYAMNWVRQASGKGLEWVGRIRSKYNNYATYYADSVKDRFTISRDDSKSTLYLQMNSLKTEDTAVYYCVRHGNFGNSYVSWFAYWGQGTLVTVSSGGGGSGGGGSGGGGSGGGGSQAVVTQEPSLTVSPGGTVTLTCRSSTGAVTTSNYANWVQQKPGQAPRGLIGGTNKRAPWTPARFSGSLLGDKAALTLSGAQPEDEAEYFCALWYSNLWVFGGGTKLTVLGGGGSDKTHTCPPCPAPELLGGPSVFLFPPKPKDTLMISRTPEVTCVVVAVSHEDPEVKFNWYVDGVEVHNAKTKPREEQYNSTYRVVSVLTVLHQDWLNGKEYKCKVSNKALAAPIEKTISKAKGQPREPQVYTLPPCREEMTKNQVSLWCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSGGGGSQVQLQESGPGLVKPSETLSLTCTVSGVSLPDYGVSWIRQPPGKGLEWIGVIWGSETTYYQSSLKSRVTISKDNSKNQVSLKLSSVTAADTAVYYCAKHYYYGGSYAMDYWGQGTLVTVSSGGGGSGGGGSGGGGSGGGGSEIVMTQSPATLSLSPGERATLSCRASQDISKYLNWYQQKPGQAPRLLIYHTSRLHSGIPARFSGSGSGTDYTLTISSLQPEDFAVYFCQQGNTLPYTFGQGTKLEIK

SEQ ID NO: 722 - protein, 214 aa, synthetic construct
  REGION 1..214: note = Description of Artificial Sequence: Synthetic polypeptide
DIQMTQSTSSLSASLGDRVTISCSASQGINNYLNWYQQKPDGTVELLIYYTSTLQSGVPSRFSGSGSGTDYSLTISNLEPEDIGTYYCQQYSKLPRTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 723 - protein, 452 aa, synthetic construct
  REGION 1..452: note = Description of Artificial Sequence: Synthetic polypeptide
QVQLQQSGSELMMPGASVKISCKATGYTFSNYWIEWVKQRPGHGLEWIGEILPGTGRTIYNEKFKGKATFTADISSNTVQMQLSSLTSEDSAVYYCARRDYYGNFYYAMDYWGQGTSVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVCTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 724 - protein, 15 aa, synthetic construct
  REGION 1..15: note = Description of Artificial Sequence: Synthetic peptide
GGGGSGGGGSGGGGS

SEQ ID NO: 725 - protein, 4 aa, synthetic construct
  REGION 1..4: note = Description of Artificial Sequence: Synthetic peptide
GFLG

SEQ ID NO: 726 - protein, 4 aa, synthetic construct
  REGION 1..4: note = Description of Artificial Sequence: Synthetic peptide
ALAL

SEQ ID NO: 727 - protein, 4 aa, synthetic construct
  REGION 1..4: note = Description of Artificial Sequence: Synthetic peptide
RGDS

SEQ ID NO: 728 - protein, 4 aa, Homo sapiens
CPSC

SEQ ID NO: 729 - protein, 4 aa, Homo sapiens
CPPC

(19) United States
(12) Patent Application Publication
(10) Pub. No.: US 2026/0365753 A1
(43) Pub. Date: Nov. 19, 2026

(54) TRISPECIFIC BINDING MOLECULES AGAINST CANCERS AND USES THEREOF

(71) Applicant: Novartis AG, Basel (CH)

(72) Inventors: Brian GRANDA, Salisbury, MA (US); Connie HONG, Somerville, MA (US)

(73) Assignee: Novartis AG, Basel (CH)

(21) Appl. No.: 19/408,836

(22) Filed: Dec. 4, 2025

Related U.S. Application Data
(63) Continuation of application No. 19/093,036, filed on Mar. 27, 2025, now abandoned.
Application No. 19/093,036 is a continuation of application No. 17/044,970, filed on Apr. 4, 2019, now abandoned.
(60) Provisional application No. 62/653,076, filed on Apr. 5, 2018.

(51) Int. Cl. C07K 16/30; A61K 39/00; A61K 47/68; C07K 16/28

(52) U.S. Cl. CPC C07K 16/30; A61K 47/68; C07K 16/2809; A61K 2039/505; C07K 2317/31; C07K 2317/55; C07K 2317/622; C07K 2318/10

(57) ABSTRACT

The present disclosure provides multispecific binding molecules that specifically bind to a first tumor-associated antigen that is expressed on cancerous B cells, a second tumor-associated antigen that is expressed on cancerous B cells, and a component of a human T-cell receptor complex, conjugates comprising the trispecific binding molecules, and pharmaceutical compositions comprising the multispecific binding molecules and the conjugates. The disclosure further provides methods of using the multispecific binding molecules to treat cancers that express the tumor-associated antigens. The disclosure yet further provides recombinant host cells engineered to express the multispecific binding molecules and methods of producing the multispecific binding molecules by culturing the host cells under conditions in which the multispecific binding molecules are expressed.

### 1. CROSS-REFERENCE TO RELATED APPLICATIONS

This application is a continuation of U.S. patent application Ser. No. 19/093,036, filed Mar. 27, 2025, now abandoned, which is a continuation of U.S. patent application Ser. No. 17/044,970, now abandoned, which is a national stage filing under 35 U.S.C. § 371 of International Application No. PCT/US2019/025760, filed Apr. 4, 2019, which claims the priority benefit of U.S. provisional application No. 62/653,076 filed Apr. 5, 2018, the contents of each of which are incorporated herein by reference in their entireties.

### 2. SEQUENCE LISTING

The instant application contains a Sequence Listing which has been submitted electronically in XML format and is hereby incorporated by reference in its entirety. The XML copy, created on Jan. 14, 2026, is named NOV-002C2_SL.xml and is 950,353 bytes in size.

### 3. INCORPORATION BY REFERENCE

All publications, patents, patent applications and other documents cited in this application are hereby incorporated by reference in their entireties for all purposes to the same extent as if each individual publication, patent, patent application or other document were individually indicated to be incorporated by reference for all purposes. In the event that there is an inconsistency between the teachings of one or more of the references incorporated herein and the present disclosure, the teachings of the present specification are intended.

### 4. BACKGROUND

Redirected targeted T-cell lysis (RTCC) is an exciting mechanism for first line treatment and refractory settings. Antibodies and antibody fragments with their exquisite selectivity have been successfully engineered in a variety of formats to allow for the dual specificities required to cross-link T-cells to a single receptor on the target cell.

There is a need for improved RTCC approaches.

### 5. SUMMARY

The present disclosure extends the principles of RTCC by providing multispecific binding molecules (“MBMs”) that engage at least two tumor-associated antigens that are expressed on cancer cells (e.g., cancerous B cells) (“TAAs”) in addition to CD3 or other component of a TCR complex on T-cells.

The present invention is based, at least in part, on the finding that engaging at least two separate TAAs in addition to a component of a TCR complex will improve the clinical outcomes of RTCC therapy of cancer, e.g., B cell malignancies by targeting a greater number of cancerous B cells than using bispecific engagers that target only a single TAA and a TCR complex component.

Accordingly, the present disclosure provides MBMs (e.g., trispecific binding molecules (“TBMs”)) that bind to (1) a first tumor-associated antigen that is expressed on cancerous B cells (“TAA 1”), (2) a second tumor-associated antigen that is expressed on cancerous B cells (“TAA 2”), and (3) CD3 or other component of a TCR complex. Because both TAA 1 and TAA 2 are tumor-associated antigens that are expressed on cancerous B cells, the designations of the tumor associated antigens of the disclosure as TAA 1 and TAA 2 are arbitrary—thus, any disclosure pertaining to TAA 1 is applicable to TAA 2 and vice versa, unless the context dictates otherwise.

The MBMs (e.g., TBMs) comprise at least three antigen-binding modules (“ABMs”) that can bind TAA 1, TAA 2 and a component of a TCR complex. Each ABM may be immunoglobulin- or non-immunoglobulin-based, and therefore the MBMs (e.g., TBMs) of the disclosure can include immunoglobulin-based ABMs, non-immunoglobulin-based ABMs, or a combination thereof. Immunoglobulin-based ABMs that can be used in the MBMs (e.g., TBMs) of the disclosure are described in Section 7.2.1 and specific embodiments 738-890, 893-1045, and 1048-1218, infra. Non-immunoglobulin-based ABMs that can be used in the MBMs (e.g., TBMs) of the disclosure are described in Section 7.2.2 and specific embodiments 891-892 and 1046-1047, infra. Further features of exemplary ABMs that bind to a component of a TCR complex are described in Section 7.5 and specific embodiments 1048-1224 and 1272-1354, infra. Further features of exemplary ABMs that bind to TAA 1 and TAA 2 are described in Section 7.6 and specific embodiments 2-737 and 1654-1663, infra.

The ABMs of a MBM (e.g., TBM) of the disclosure (or portions thereof) can be connected to each other, for example, by short peptide linkers or by an Fc domain. Methods and components for connecting ABMs to form a MBM are described in Section 7.3 and specific embodiments 1219-1271 and 1355-1550, infra.

MBMs (e.g., TBMs) of the disclosure have at least three ABMs (e.g., a TBM is at least trivalent), but can also have more than three ABMs. For example, a MBM (e.g., a TBM) can have four ABMs (i.e., is tetravalent), five ABMs (i.e., is pentavalent), or six ABMs (i.e., is hexavalent), provided that the MBM has at least one ABM that can bind TAA 1, at least one ABM that can bind TAA 2, and at least one ABM that can bind a component of a TCR complex. Exemplary trivalent, tetravalent, pentavalent, and hexavalent TBM configurations are shown in FIG. 1 and described in Section 7.4 and specific embodiments 1552-1572, 1574-1602, 1604-1615, and 1617-1653 infra.

The disclosure further provides nucleic acids encoding the MBMs of the disclosure (either in a single nucleic acid or a plurality of nucleic acids) and recombinant host cells and cell lines engineered to express the nucleic acids and MBMs of the disclosure. Exemplary nucleic acids, host cells, and cell lines are described in Section 7.7 and specific embodiments 1784-1792, infra.

The present disclosure further provides drug conjugates comprising the MBMs of the disclosure. Such conjugates are referred to herein as “antibody-drug conjugates” or “ADCs” for convenience, notwithstanding that some or all of the ABMs can be non-immunoglobulin domains. Examples of ADCs are described in Section 7.8 and specific embodiments 1665-1704, infra.

Pharmaceutical compositions comprising the MBMs and ADCs of the disclosure are also provided. Examples of pharmaceutical compositions are described in Section 7.9 and specific embodiment 1705, infra.

Further provided herein are methods of using the MBMs, the ADCs, and the pharmaceutical compositions of the disclosure, for example for treating B cell malignancies and autoimmune disorders. Exemplary methods are described in Section 7.10 and specific embodiments 1706-1729 and 1750-1783, infra.

The disclosure further provides methods of using the MBMs, the ADCs, and the pharmaceutical compositions of the disclosure in combination with other agents and therapies. Exemplary agents, therapies, and methods of combination therapy are described in Section 7.11 and specific embodiments 1730-1749, infra.

### 6. BRIEF DESCRIPTION OF THE FIGURES

FIGS. 1A-1U: Exemplary TBM configurations. FIG. 1A illustrates components of the exemplary TBM configurations illustrated in FIGS. 1B-1U. Not all regions connecting the different domains of each chain are illustrated (e.g., the linker connecting the VH and VL domains of an scFv, the hinge connecting the CH2 and CH3 domains of an Fc, etc., are omitted). FIG. 1B-1O illustrates trivalent TBMs; FIGS. 1P-1R illustrate tetravalent TBMs; FIG. 1S illustrates a pentavalent TBM, and FIGS. 1T-1U illustrates hexavalent TBMs.

FIG. 2: Schematics of the bispecific and trispecific constructs of Example 1.

FIGS. 3A-G: Schematics of the bispecific and trispecific constructs of Example 2.

FIG. 4: Schematics of the bispecific and trispecific constructs of Example 3.

FIG. 5: Cytotoxicity assay results (Example 1).

FIG. 6: Percentage of tumor cell lysis when co-culturing target cells with human T cells in the presence of bispecific and tri-specific antibodies (Example 2).

FIG. 7: Percentage of tumor cell lysis when co-culturing Ramos with human T cells in the presence of bispecific and tri-specific antibodies (Example 2).

FIG. 8: EC50 of bispecific and tri-specific antibodies measured in three different cell lines using RTCC assay (Example 2).

FIGS. 9A-9B: Cell surface expression of BCMA (FIG. 9A) and CD138 (FIG. 9B) on MM1s cells measured by flow cytometry (Example 3).

FIG. 10: Impact of added soluble BCMA ECD on the activity of BCMA×CD3 bispecific and CD138×BCMA×CD3 trispecific Abs in an MM1S RTCC assay (Example 3).

FIG. 11: RTCC assay EC50 values in MM cell line MM1S comparing the effect of soluble BCMA on the activity of different MM targeting CD3 multi-specific antibodies (Example 3).

### 7. DETAILED DESCRIPTION

### 7.1. Definitions

As used herein, the following terms are intended to have the following meanings:

Antigen-binding module: The term “antigen-binding module” or “ABM” as used herein refers to a portion of a MBM of the disclosure that has the ability to bind to an antigen non-covalently, reversibly and specifically. An ABM may be immunoglobulin- or non-immunoglobulin-based. As used herein, the terms “ABM1” and “TAA 1 ABM” (and the like) refers to an ABM that binds specifically to TAA 1, the terms “ABM2” and “TAA 2 ABM” (and the like) refers to an ABM that binds specifically to TAA 2, and the terms “ABM3” and “TCR ABM” (and the like) refers to an ABM that binds specifically to a component of a TCR. The terms ABM1, ABM2, and ABM3 are used merely for convenience and are not intended to convey any particular configuration of a MBM. In some embodiments, a TCR ABM binds to CD3 (referred to herein a “CD3 ABM” or the like). Accordingly, disclosures relating to ABM3 and TCR ABMs are also applicable to CD3 ABMs.

Antibody: The term “antibody” as used herein refers to a polypeptide (or set of polypeptides) of the immunoglobulin family that is capable of binding an antigen non-covalently, reversibly and specifically. For example, a naturally occurring “antibody” of the IgG type is a tetramer comprising at least two heavy (H) chains and two light (L) chains inter-connected by disulfide bonds. Each heavy chain is comprised of a heavy chain variable region (abbreviated herein as VH) and a heavy chain constant region. The heavy chain constant region is comprised of three domains, CH1, CH2 and CH3. Each light chain is comprised of a light chain variable region (abbreviated herein as VL) and a light chain constant region. The light chain constant region is comprised of one domain (abbreviated herein as CL). The VH and VL regions can be further subdivided into regions of hypervariability, termed complementarity determining regions (CDR), interspersed with regions that are more conserved, termed framework regions (FR). Each VH and VL is composed of three CDRs and four FRs arranged from amino-terminus to carboxy-terminus in the following order: FR1, CDR1, FR2, CDR2, FR3, CDR3, FR4. The variable regions of the heavy and light chains contain a binding domain that interacts with an antigen. The constant regions of the antibodies may mediate the binding of the immunoglobulin to host tissues or factors, including various cells of the immune system (e.g., effector cells) and the first component (Clq) of the classical complement system. The term “antibody” includes, but is not limited to, monoclonal antibodies, human antibodies, humanized antibodies, camelised antibodies, chimeric antibodies, bispecific or multispecific antibodies and anti-idiotypic (anti-Id) antibodies (including, e.g., anti-Id antibodies to antibodies of the disclosure). The antibodies can be of any isotype/class (e.g., IgG, IgE, IgM, IgD, IgA and IgY) or subclass (e.g., IgG1, IgG2, IgG3, IgG4, IgA1 and IgA2).

Both the light and heavy chains are divided into regions of structural and functional homology. The terms “constant” and “variable” are used functionally. In this regard, it will be appreciated that the variable domains of both the light (VL) and heavy (VH) chain portions determine antigen recognition and specificity. Conversely, the constant domains of the light chain (CL) and the heavy chain (CH1, CH2 or CH3) confer important biological properties such as secretion, transplacental mobility, Fc receptor binding, complement binding, and the like. By convention the numbering of the constant region domains increases as they become more distal from the antigen-binding site or amino-terminus of the antibody. The N-terminus is a variable region and at the C-terminus is a constant region; the CH3 and CL domains actually comprise the carboxy-terminus of the heavy and light chain, respectively.

Antibody fragment: The term “antibody fragment” of an antibody as used herein refers to one or more portions of an antibody. In some embodiments, these portions are part of the contact domain(s) of an antibody. In some other embodiments, these portion(s) are antigen-binding fragments that retain the ability of binding an antigen non-covalently, reversibly and specifically, sometimes referred to herein as the “antigen-binding fragment”, “antigen-binding fragment thereof,” “antigen-binding portion”, and the like. Examples of binding fragments include, but are not limited to, single-chain Fvs (scFv), a Fab fragment, a monovalent fragment consisting of the VL, VH, CL and CH1 domains; a F(ab)2 fragment, a bivalent fragment comprising two Fab fragments linked by a disulfide bridge at the hinge region; a Fd fragment consisting of the VH and CH1 domains; a Fv fragment consisting of the VL and VH domains of a single arm of an antibody; a dAb fragment (Ward et al., (1989) Nature 341:544-546), which consists of a VH domain; and an isolated complementarity determining region (CDR). Thus, the term “antibody fragment” encompasses both proteolytic fragments of antibodies (e.g., Fab and F(ab)2 fragments) and engineered proteins comprising one or more portions of an antibody (e.g., an scFv).

Antibody fragments can also be incorporated into single domain antibodies, maxibodies, minibodies, intrabodies, diabodies, triabodies, tetrabodies, v-NAR and bis-scFv (see, e.g., Hollinger and Hudson, 2005, Nature Biotechnology 23: 1126-1136). Antibody fragments can be grafted into scaffolds based on polypeptides such as Fibronectin type III (Fn3) (see U.S. Pat. No. 6,703,199, which describes fibronectin polypeptide monobodies).

Antibody fragments can be incorporated into single chain molecules comprising a pair of tandem Fv segments (for example, VH—CH1-VH—CH1) which, together with complementary light chain polypeptides (for example, VL-VC-VL-VC), form a pair of antigen-binding regions (Zapata et al., 1995, Protein Eng. 8:1057-1062; and U.S. Pat. No. 5,641,870).

Antigen-binding domain: The term “antigen-binding domain” refers to a portion of a molecule that has the ability to bind to an antigen non-covalently, reversibly and specifically. Exemplary antigen-binding domains include antigen-binding fragments and portions of both immunoglobulin and non-immunoglobulin based scaffolds that retain the ability of binding an antigen non-covalently, reversibly and specifically. As used herein, the term “antigen-binding domain” encompasses antibody fragments that retain the ability of binding an antigen non-covalently, reversibly and specifically.

Half Antibody: The term “half antibody” refers to a molecule that comprises at least one ABM or ABM chain and can associate with another molecule comprising an ABM or ABM chain through, e.g., a disulfide bridge or molecular interactions (e.g., knob-in-hole interactions between Fc heterodimers). A half antibody can be composed of one polypeptide chain or more than one polypeptide chains (e.g., the two polypeptide chains of a Fab). In an embodiment, a half-antibody comprises an Fc region.

An example of a half antibody is a molecule comprising a heavy and light chain of an antibody (e.g., an IgG antibody). Another example of a half antibody is a molecule comprising a first polypeptide comprising a VL domain and a CL domain, and a second polypeptide comprising a VH domain, a CH1 domain, a hinge domain, a CH2 domain, and a CH3 domain, where the VL and VH domains form an ABM. Yet another example of a half antibody is a polypeptide comprising an scFv domain, a CH2 domain and a CH3 domain.

A half antibody might include more than one ABM, for example a half-antibody comprising (in N- to C-terminal order) an scFv domain, a CH2 domain, a CH3 domain, and another scFv domain.

Half antibodies might also include an ABM chain that when associated with another ABM chain in another half antibody forms a complete ABM.

Thus, a MBM (e.g., a TBM) can comprise one, more typically two, or even more than two half antibodies, and a half antibody can comprise one or more ABMs or ABM chains.

In some MBMs, a first half antibody will associate, e.g., heterodimerize, with a second half antibody. In other MBMs, a first half antibody will be covalently linked to a second half antibody, for example through disulfide bridges or chemical crosslinking. In yet other MBMs, a first half antibody will associate with a second half antibody through both covalent attachments and non-covalent interactions, for example disulfide bridges and knob-in-hole interactions.

The term “half antibody” is intended for descriptive purposes only and does not connote a particular configuration or method of production. Descriptions of a half antibody as a “first” half antibody, a “second” half antibody, a “left” half antibody, a “right” half antibody or the like are merely for convenience and descriptive purposes.

Complementarity Determining Region: The terms “complementarity determining region” or “CDR,” as used herein, refer to the sequences of amino acids within antibody variable regions which confer antigen specificity and binding affinity. For example, in general, there are three CDRs in each heavy chain variable region (e.g., CDR-H1, CDR-H2, and CDR-H3) and three CDRs in each light chain variable region (CDR-L1, CDR-L2, and CDR-L3). The precise amino acid sequence boundaries of a given CDR can be determined using any of a number of well-known schemes, including those described by Kabat et al., 1991, “Sequences of Proteins of Immunological Interest,” 5th Ed. Public Health Service, National Institutes of Health, Bethesda, MD (“Kabat” numbering scheme), Al-Lazikani et al., 1997, JMB 273:927-948 (“Chothia” numbering scheme) and ImMunoGenTics (IMGT) numbering (Lefranc, 1999, The Immunologist 7:132-136 (1999); Lefranc et al., 2003, Dev. Comp. Immunol. 27:55-77 (“IMGT” numbering scheme). For example, for classic formats, under Kabat, the CDR amino acid residues in the heavy chain variable domain (VH) are numbered 31-35 (CDR-H1), 50-65 (CDR-H2), and 95-102 (CDR-H3); and the CDR amino acid residues in the light chain variable domain (VL) are numbered 24-34 (CDR-L1), 50-56 (CDR-L2), and 89-97 (CDR-L3). Under Chothia, the CDR amino acids in the VH are numbered 26-32 (CDR-H1), 52-56 (CDR-H2), and 95-102 (CDR-H3); and the amino acid residues in VL are numbered 26-32 (CDR-L1), 50-52 (CDR-L2), and 91-96 (CDR-L3). By combining the CDR definitions of both Kabat and Chothia, the CDRs consist of amino acid residues 26-35 (CDR-H1), 50-65 (CDR-H2), and 95-102 (CDR-H3) in human VH and amino acid residues 24-34 (CDR-L1), 50-56 (CDR-L2), and 89-97 (CDR-L3) in human VL. Under IMGT the CDR amino acid residues in the VH are numbered approximately 26-35 (CDR-H1), 51-57 (CDR-H2) and 93-102 (CDR-H3), and the CDR amino acid residues in the VL are numbered approximately 27-32 (CDR-L1), 50-52 (CDR-L2), and 89-97 (CDR-L3) (numbering according to “Kabat”). Under IMGT, the CDR regions of an antibody can be determined using the program IMGT/DomainGap Align.

Single Chain Fv or scFv: The term “single-chain Fv” or “scFv” as used herein refers to antibody fragments comprise the VH and VL domains of an antibody, where these domains are present in a single polypeptide chain. The Fv polypeptide can further comprise a polypeptide linker between the VH and VL domains which enables the scFv to form the desired structure for antigen-binding. For a review of scFv see Pluckthun in The Pharmacology of Monoclonal Antibodies, vol. 113, Rosenburg and Moore eds., (1994) Springer-Verlag, New York, pp. 269-315.

Diabody: The term “diabody” as used herein refers to small antibody fragments with two antigen-binding sites, typically formed by pairing of scFv chains. Each scFv comprises a heavy chain variable domain (VH) connected to a light chain variable domain (VL) in the same polypeptide chain (VH-VL, where the VH is either N-terminal or C-terminal to the VL). Unlike a typical scFv in which the VH and VL are separated by a linker that allows the VH and VL on the same polypeptide chain to pair and form an antigen-binding domain, diabodies typically comprise a linker that is too short to allow pairing between the VH and VL domains on the same chain, forcing the VH and VL domains to pair with the complementary domains of another chain and create two antigen-binding sites. Diabodies are described more fully in, for example, EP 404,097; WO 93/11161; and Hollinger et al., 1993, Proc. Natl. Acad. Sci. USA 90:6444-6448.

Fv: The term “Fv” refers to the minimum antibody fragment derivable from an immunoglobulin that contains a complete target recognition and binding site. This region consists of a dimer of one heavy and one light chain variable domain in a tight, noncovalent association (VH-VL dimer). It is in this configuration that the three CDRs of each variable domain interact to define a target binding site on the surface of the VH-VL dimer. Often, the six CDRs confer target binding specificity to the antibody. However, in some instances even a single variable domain (or half of an Fv comprising only three CDRs specific for a target) can have the ability to recognize and bind target. The reference to a VH-VL dimer herein is not intended to convey any particular configuration. By way of example and not limitation, the VH and VL can come together in any configuration described herein to form a half antibody, or can each be present on a separate half antibody and come together to form an antigen binding domain when the separate half antibodies associate, for example to form a TBM of the disclosure. When present on a single polypeptide chain (e.g., a scFv), the VH and be N-terminal or C-terminal to the VL.

Multispecific binding molecules: The term “multispecific binding molecules” or “MBMs” refers to molecules that specifically bind to at least two antigens and comprise two or more antigen-binding domains. The antigen-binding domains can each independently be an antibody fragment (e.g., scFv, Fab, nanobody), a ligand, or a non-antibody derived binder (e.g., fibronectin, Fynomer, DARPin).

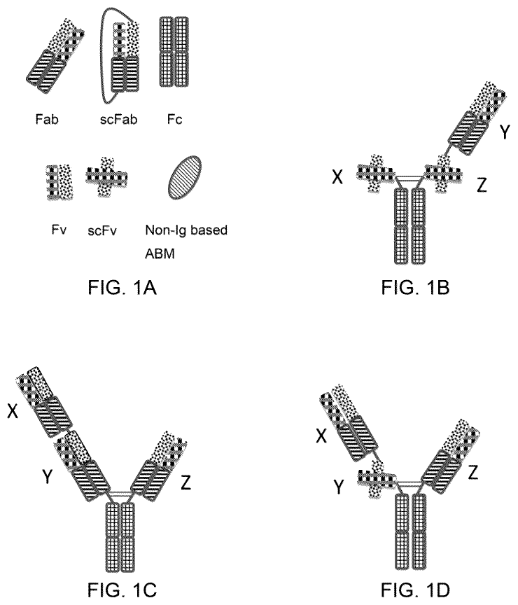

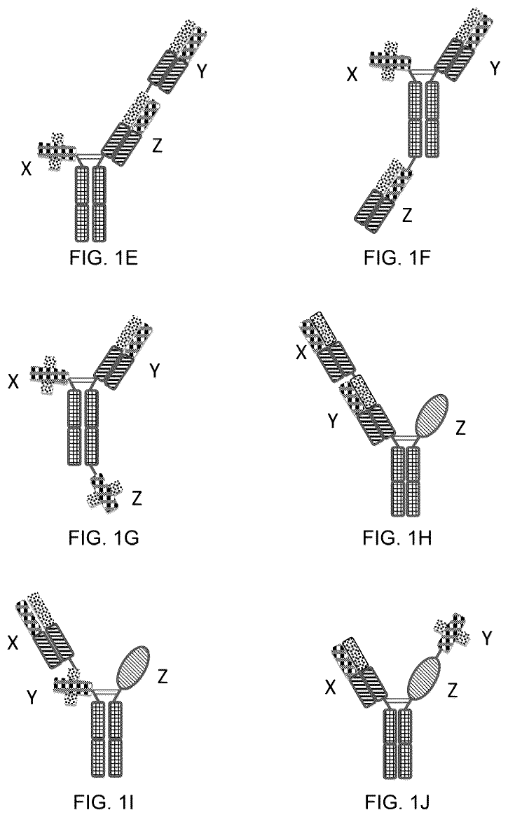

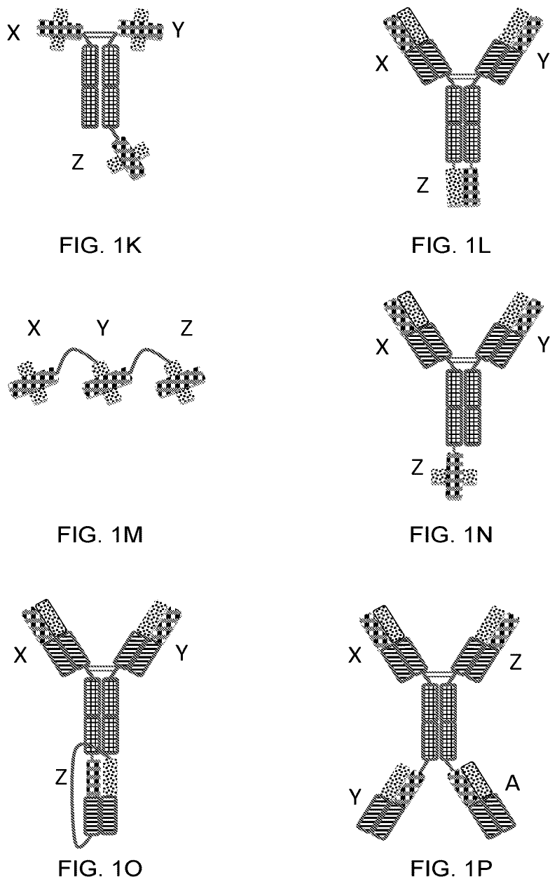

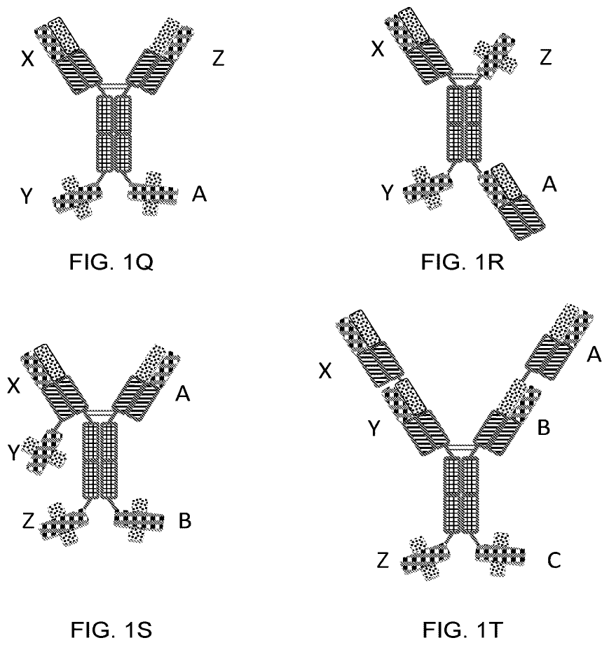

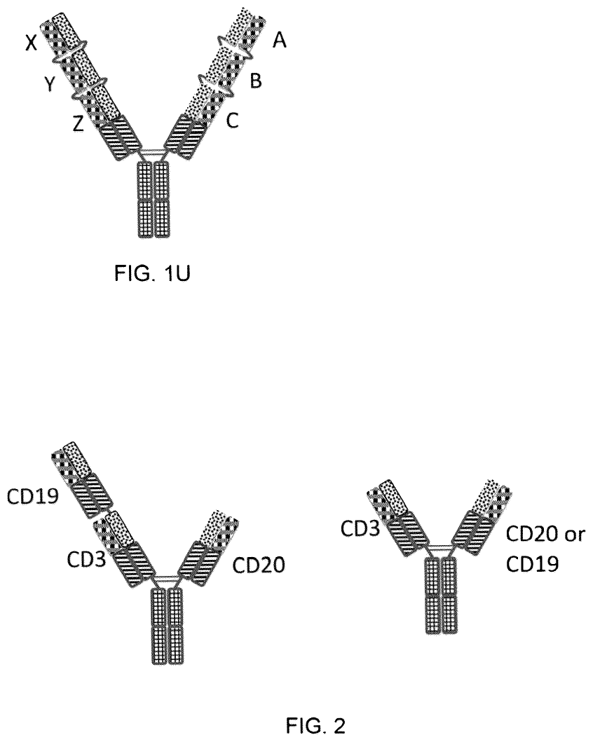

Trispecific binding molecules: The term “trispecific binding molecules” or “TBMs” refers to molecules that specifically bind to three antigens and comprise three or more antigen-binding domains. The TBMs of the disclosure comprise at least one antigen-binding domain which is specific for a component of a TCR complex, at least one antigen-binding domain which is specific for TAA 1, and at least one antigen-binding domain which is specific for TAA 2. The antigen-binding domains can each independently be an antibody fragment (e.g., scFv, Fab, nanobody), a ligand, or a non-antibody derived binder (e.g., fibronectin, Fynomer, DARPin). Representative TBMs are illustrated in FIG. 1. TBMs can comprise one, two, three, four or even more polypeptide chains. For example, the TBM illustrated in FIG. 1M comprises a single polypeptide chain comprising three scFvs connected by ABM linkers one a single polypeptide chain. The TBM illustrated in FIG. 1K comprises two polypeptide chains comprising three scFvs connected by, inter alia, an Fc domain. The TBM illustrated in FIG. 1J comprises three polypeptide chains forming an scFv, a ligand, and a Fab connected by, inter alia, an Fc domain. The TBM illustrated in FIG. 1C comprises four polypeptide chains forming three Fabs connected by, inter alia, an Fc domain. The TBM illustrated in FIG. 1T comprises 6 polypeptide chains forming four Fabs and two scFvs connected by, inter alia, an Fc domain.

VH: The term “VH” refers to the variable region of an immunoglobulin heavy chain of an antibody, including the heavy chain of an Fv, scFv, dsFv or Fab.

VL: The term “VL” refers to the variable region of an immunoglobulin light chain, including the light chain of an Fv, scFv, dsFv or Fab.

Operably linked: The term “operably linked” refers to a functional relationship between two or more peptide or polypeptide domains or nucleic acid (e.g., DNA) segments. In the context of a fusion protein or other polypeptide, the term “operably linked” means that two or more amino acid segments are linked so as to produce a functional polypeptide. For example, in the context of a TBM of the disclosure, separate ABMs (or chains of an ABM) can be through peptide linker sequences. In the context of a nucleic acid encoding a fusion protein, such as a polypeptide chain of a TBM of the disclosure, “operably linked” means that the two nucleic acids are joined such that the amino acid sequences encoded by the two nucleic acids remain in-frame. In the context of transcriptional regulation, the term refers to the functional relationship of a transcriptional regulatory sequence to a transcribed sequence. For example, a promoter or enhancer sequence is operably linked to a coding sequence if it stimulates or modulates the transcription of the coding sequence in an appropriate host cell or other expression system.

Associated: The term “associated” in the context of a MBM refers to a functional relationship between two or more polypeptide chains. In particular, the term “associated” means that two or more polypeptides are associated with one another, e.g., non-covalently through molecular interactions or covalently through one or more disulfide bridges or chemical cross-linkages, so as to produce a functional MBM (e.g., a TBM) in which ABM1, ABM2 and ABM3 can bind their respective targets. Examples of associations that might be present in a MBM of the disclosure include (but are not limited to) associations between Fc regions in an Fc domain (homodimeric or heterodimeric as described in Section 7.3.1.5), associations between VH and VL regions in a Fab or Fv, and associations between CH1 and CL in a Fab.

ABM chain: Individual ABMs can exist as one (e.g., in the case of an scFv) polypeptide chain or form through the association of more than one polypeptide chains (e.g., in the case of a Fab). As used herein, the term “ABM chain” refers to all or a portion of an ABM that exists on a single polypeptide chain. The use of the term “ABM chain” is intended for convenience and descriptive purposes only and does not connote a particular configuration or method of production.

Host cell or recombinant host cell: The terms “host cell” or “recombinant host cell” refer to a cell that has been genetically-engineered, e.g., through introduction of a heterologous nucleic acid. It should be understood that such terms are intended to refer not only to the particular subject cell but to the progeny of such a cell. Because certain modifications may occur in succeeding generations due to either mutation or environmental influences, such progeny may not, in fact, be identical to the parent cell, but are still included within the scope of the term “host cell” as used herein. A host cell may carry the heterologous nucleic acid transiently, e.g., on an extrachromosomal heterologous expression vector, or stably, e.g., through integration of the heterologous nucleic acid into the host cell genome. For purposes of expressing a MBM of the disclosure, a host cell can be a cell line of mammalian origin or mammalian-like characteristics, such as monkey kidney cells (COS, e.g., COS-1, COS-7), HEK293, baby hamster kidney (BHK, e.g., BHK21), Chinese hamster ovary (CHO), NSO, PerC6, BSC-1, human hepatocellular carcinoma cells (e.g., Hep G2), SP2/0, HeLa, Madin-Darby bovine kidney (MDBK), myeloma and lymphoma cells, or derivatives and/or engineered variants thereof. The engineered variants include, e.g., glycan profile modified and/or site-specific integration site derivatives.

Sequence identity: The term percent “identity,” in the context of two or more nucleic acids or polypeptide sequences, refers to two or more sequences that are the same. Two sequences are “substantially identical” if two sequences have a specified percentage of amino acid residues or nucleotides that are the same (e.g., 60% identity, optionally 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% identity over a specified region, or, when not specified, over the entire sequence), when compared and aligned for maximum correspondence over a comparison window, or designated region as measured using one of the following sequence comparison algorithms or by manual alignment and visual inspection. Optionally, the identity exists over a region that is at least about 50 nucleotides (or, in the case of a peptide or polypeptide, at least about 10 amino acids) in length, or in some cases over a region that is 100 to 500 or 1000 or more nucleotides (or 20, 50, 200 or more amino acids) in length.

For sequence comparison, typically one sequence acts as a reference sequence, to which test sequences are compared. When using a sequence comparison algorithm, test and reference sequences are entered into a computer, subsequence coordinates are designated, if necessary, and sequence algorithm program parameters are designated. Default program parameters can be used, or alternative parameters can be designated. The sequence comparison algorithm then calculates the percent sequence identities for the test sequences relative to the reference sequence, based on the program parameters. Methods of alignment of sequences for comparison are well known in the art. Optimal alignment of sequences for comparison can be conducted, e.g., by the local homology algorithm of Smith and Waterman, 1970, Adv. Appl. Math. 2:482c, by the homology alignment algorithm of Needleman and Wunsch, 1970, J. Mol. Biol. 48:443, by the search for similarity method of Pearson and Lipman, 1988, Proc. Nat'l. Acad. Sci. USA 85:2444, by computerized implementations of these algorithms (GAP, BESTFIT, FASTA, and TFASTA in the Wisconsin Genetics Software Package, Genetics Computer Group, 575 Science Dr., Madison, WI), or by manual alignment and visual inspection (see, e.g., Brent et al., 2003, Current Protocols in Molecular Biology).

Two examples of algorithms that are suitable for determining percent sequence identity and sequence similarity are the BLAST and BLAST 2.0 algorithms, which are described in Altschul et al., 1977, Nuc. Acids Res. 25:3389-3402; and Altschul et al., 1990, J. Mol. Biol. 215:403-410, respectively. Software for performing BLAST analyses is publicly available through the National Center for Biotechnology Information.

The percent identity between two amino acid sequences can also be determined using the algorithm of Meyers and Miller, 1988, Comput. Appl. Biosci. 4:11-17, which has been incorporated into the ALIGN program (version 2.0), using a PAM120 weight residue table, a gap length penalty of 12 and a gap penalty of 4. In addition, the percent identity between two amino acid sequences can be determined using the Needleman and Wunsch, 1970, J. Mol. Biol. 48:444-453) algorithm which has been incorporated into the GAP program in the GCG software package (available at gcg.com), using either a Blossom 62 matrix or a PAM250 matrix, and a gap weight of 16, 14, 12, 10, 8, 6, or 4 and a length weight of 1, 2, 3, 4, 5, or 6.

Conservative Sequence Modifications: The term “conservative sequence modifications” refers to amino acid modifications that do not significantly affect or alter the binding characteristics of a MBM or a component thereof (e.g., an ABM or an Fc region). Such conservative modifications include amino acid substitutions, additions and deletions. Modifications can be introduced into a MBM of the disclosure by standard techniques known in the art, such as site-directed mutagenesis and PCR-mediated mutagenesis. Conservative amino acid substitutions are ones in which the amino acid residue is replaced with an amino acid residue having a similar side chain. Families of amino acid residues having similar side chains have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine, tryptophan), nonpolar side chains (e.g., alanine, valine, leucine, isoleucine, proline, phenylalanine, methionine), beta-branched side chains (e.g., threonine, valine, isoleucine) and aromatic side chains (e.g., tyrosine, phenylalanine, tryptophan, histidine). Thus, one or more amino acid residues within a MBM of the disclosure can be replaced with other amino acid residues from the same side chain family and the altered MBM can be tested for, e.g., binding to target molecules and/or effective heterodimerization and/or effector function.

Mutation or modification: The terms “mutation” and “modification” in the context of a polypeptide as used herein can include substitution, addition or deletion of one or more amino acids.

Antibody Numbering Systems: In the present specification, the references to numbered amino acid residues in antibody domains are based on the EU numbering system unless otherwise specified (for example, in Tables 8B and 8C). This system was originally devised by Edelman et al., 1969, Proc. Nat'l Acad. Sci. USA 63:78-85 and is described in detail in Kabat et al., 1991, in Sequences of Proteins of Immunological Interest, US Department of Health and Human Services, NIH, USA.

dsFv: The term “dsFv” refers to disulfide-stabilized Fv fragments. In a dsFv, a VH and VL are connected by an interdomain disulfide bond. To generate such molecules, one amino acid each in the framework region of in VH and VL are mutated to a cysteine, which in turn form a stable interchain disulfide bond. Typically, position 44 in the VH and position 100 in the VL are mutated to cysteines. See Brinkmann, 2010, Antibody Engineering 181-189, DOI:10.1007/978-3-642-01147-4_14. The term dsFv encompasses both what is known in the art a dsFv (a molecule in which the VH and VL are connected by an interchain disulfide bond but not a linker peptide) or scdsFv (a molecule in which the VH and VL are connected by a linker as well as an interchain disulfide bond).

Tandem of VH Domains: The term “a tandem of VH domains (or VHs)” as used herein refers to a string of VH domains, consisting of multiple numbers of identical VH domains of an antibody. Each of the VH domains, except the last one at the end of the tandem, has its C-terminus connected to the N-terminus of another VH domain with or without a linker. A tandem has at least 2 VH domains, and in particular embodiments of the TBMs of the disclosure has 3, 4, 5, 6, 7, 8, 9, or 10 VH domains. The tandem of VH can be produced by joining the encoding nucleic acids of each VH domain in a desired order using recombinant methods with or without a linker (e.g., as described in Section 7.3.3) that enables them to be made as a single polypeptide chain. The N-terminus of the first VH domain in the tandem is defined as the N-terminus of the tandem, while the C-terminus of the last VH domain in the tandem is defined as the C-terminus of the tandem.

Tandem of VL Domains: The term “a tandem of VL domains (or VLs)” as used herein refers to a string of VL domains, consisting of multiple numbers of identical VL domains of an antibody. Each of the VL domains, except the last one at the end of the tandem, has its C-terminus connected to the N-terminus of another VL with or without a linker. A tandem has at least 2 VL domains, and in particular embodiments of the TBMs of the disclosure has 3, 4, 5, 6, 7, 8, 9, or 10 VL domains. The tandem of VL can be produced by joining the encoding nucleic acids of each VL domain in a desired order using recombinant methods with or without a linker (e.g., as described in Section 7.3.3) that enables them to be made as a single polypeptide chain. The N-terminus of the first VL domain in the tandem is defined as the N-terminus of the tandem, while the C-terminus of the last VL domain in the tandem is defined as the C-terminus of the tandem.

Monovalent: The term “monovalent” as used herein in the context of an antigen-binding molecule refers to an antigen-binding molecule that has a single antigen-binding domain.

Bivalent: The term “bivalent” as used herein in the context of an antigen-binding molecule refers to an antigen-binding molecule that has two antigen-binding domains. The domains can be the same or different. Accordingly, a bivalent antigen-binding molecule can be monospecific or bispecific.

Trivalent: The term “trivalent” as used herein in the context of an antigen-binding molecule (e.g., a TBM) refers to an antigen-binding molecule that has three antigen-binding domains. The TBMs of the disclosure are trispecific and specifically bind to TAA 1, TAA 2, and a component of a TCR complex. Accordingly, the trivalent TBMs of the disclosure have at least three antigen-binding domains that each bind to a different antigen. Examples of trivalent TBMs of the disclosure are shown schematically in FIGS. 1B-1U.

Tetravalent: The term “tetravalent” as used herein in the context of an antigen-binding molecule (e.g., a TBM) refers to an antigen-binding molecule that has four antigen-binding domains. The TBMs of the disclosure are trispecific and specifically bind to TAA 1, TAA 2, and a component of a TCR complex. Accordingly, the tetravalent TBMs of the disclosure generally have two antigen-binding domains that bind to the same antigen (e.g., TAA 1 or TAA 2) and two antigen-binding domains that each bind to a separate antigen (e.g., a component of a TCR complex and either TAA 1 or TAA 2). Examples of tetravalent TBMs of the disclosure are shown schematically in FIGS. 1P-1R.

Pentavalent: The term “pentavalent” as used herein in the context of an antigen-binding molecule (e.g., a TBM) refers to an antigen-binding molecule that has five antigen-binding domains. The TBMs of the disclosure are trispecific and specifically bind to TAA 1, TAA 2, and a component of a TCR complex. Accordingly, the pentavalent TBMs of the disclosure generally have either (a) two pairs of antigen-binding domains that each bind to the same antigen and a single antigen-binding domain that binds to the third antigen or (b) three antigen-binding domains that bind to the same antigen and two antigen-binding domains that each bind to a separate antigen. An example of a pentavalent TBM of the disclosure is shown schematically in FIG. 1S.

Hexavalent: The term “hexavalent” as used herein in the context of an antigen-binding molecule (e.g., a TBM) refers to an antigen-binding molecule that has six antigen-binding domains. The TBMs of the disclosure are trispecific and specifically bind to TAA 1, TAA 2, and a component of a TCR complex. The hexavalent TBMs of the disclosure generally have three pairs of antigen-binding domains that each bind to the same antigen, although different configurations (e.g., three antigen-binding domains that bind to TAA 1, two antigen-binding domains that bind to TAA 2, and one antigen-binding domain that binds to a component of a TCR complex, or three antigen-binding domains that bind to TAA 1, two antigen-binding domains that bind to a component of a TCR complex, and one antigen-binding domain that binds to TAA 2) are within the scope of the disclosure. Examples of hexavalent TBMs of the disclosure are shown schematically in FIGS. 1T-1U.

Specifically (or selectively) binds: The term “specifically (or selectively) binds” to an antigen or an epitope refers to a binding reaction that is determinative of the presence of a cognate antigen or an epitope in a heterogeneous population of proteins and other biologics. The binding reaction can be but need not be mediated by an antibody or antibody fragment, but can also be mediated by, for example, any type of ABM described in Section 7.2, such as a ligand, a DARPin, etc. An ABM of the disclosure typically also has a dissociation rate constant (KD) (koff/kon) of less than 5×10⁻²M, less than 10⁻²M, less than 5×10⁻³M, less than 10⁻³M, less than 5×10⁻⁴M, less than 10⁻⁴M, less than 5×10⁻⁵M, less than 10⁻⁵M, less than 5×10⁻⁶M, less than 10⁻⁶M, less than 5×10⁻⁷M, less than 10⁻⁷M, less than 5×10⁻⁸M, less than 10⁻⁸M, less than 5×10⁻⁹M, or less than 10⁻⁹M, and binds to the target antigen with an affinity that is at least two-fold greater than its affinity for binding to a non-specific antigen (e.g., HSA). The term “specifically binds” does not exclude cross-species reactivity. For example, an antigen-binding module (e.g., an antigen-binding fragment of an antibody) that “specifically binds” to an antigen from one species may also “specifically bind” to that antigen in one or more other species. Thus, such cross-species reactivity does not itself alter the classification of an antigen-binding module as a “specific” binder. In certain embodiments, an antigen-binding module of the disclosure (e.g., ABM1, ABM2 and/or ABM3) that specifically binds to a human antigen has cross-species reactivity with one or more non-human mammalian species, e.g., a primate species (including but not limited to one or more of Macaca fascicularis, Macaca mulatta, and Macaca nemestrina) or a rodent species, e.g., Mus musculus. In other embodiments, the antigen-binding module of the disclosure (e.g., ABM1, ABM2 and/or ABM3) does not have cross-species reactivity.

Monoclonal Antibody: The term “monoclonal antibody” as used herein refers to polypeptides, including antibodies, antibody fragments, molecules (including TBMs), etc. that are derived from the same genetic source.

Humanized: The term “humanized” forms of non-human (e.g., murine) antibodies are chimeric antibodies that contain minimal sequence derived from non-human immunoglobulin. For the most part, humanized antibodies are human immunoglobulins (recipient antibody) in which residues from a hypervariable region of the recipient are replaced by residues from a hypervariable region of a non-human species (donor antibody) such as mouse, rat, rabbit or non-human primate having the desired specificity, affinity, and capacity. In some instances, framework region (FR) residues of the human immunoglobulin are replaced by corresponding non-human residues. Furthermore, humanized antibodies may comprise residues that are not found in the recipient antibody or in the donor antibody. These modifications are made to further refine antibody performance. In general, the humanized antibody will comprise substantially all of at least one, and typically two, variable domains, in which all or substantially all of the hypervariable loops correspond to those of a non-human immunoglobulin and all or substantially all of the FRs are those of a human immunoglobulin lo sequence. The humanized antibody optionally will also comprise at least a portion of an immunoglobulin constant region (Fc), typically that of a human immunoglobulin. For further details, see Jones et al., 1986, Nature 321:522-525; Riechmann et al., 1988, Nature 332:323-329; and Presta, 1992, Curr. Op. Struct. Biol. 2:593-596. See also the following review articles and references cited therein: Vaswani and Hamilton, 1998, Ann. Allergy, Asthma & Immunol. 1:105-115; Harris, 1995, Biochem. Soc. Transactions 23:1035-1038; Hurle and Gross, 1994, Curr. Op. Biotech. 5:428-433.

Human Antibody: The term “human antibody” as used herein includes antibodies having variable regions in which both the framework and CDR regions are derived from sequences of human origin. Furthermore, if the antibody contains a constant region, the constant region also is derived from such human sequences, e.g., human germline sequences, or mutated versions of human germline sequences or antibody containing consensus framework sequences derived from human framework sequences analysis, for example, as described in Knappik et al., 2000, J Mol Biol 296, 57-86. The structures and locations of immunoglobulin variable domains, e.g., CDRs, may be defined using well known numbering schemes, e.g., the Kabat numbering scheme, the Chothia numbering scheme, or a combination of Kabat and Chothia (see, e.g., Lazikani et al., 1997, J. Mol. Bio. 273:927 948; Kabat et al., 1991, Sequences of Proteins of Immunological Interest, 5th edit., NIH Publication no. 91-3242 U.S. Department of Health and Human Services; Chothia et al., 1987, J. Mol. Biol. 196:901-917; Chothia et al., 1989, Nature 342:877-883).

Human antibodies may include amino acid residues not encoded by human sequences (e.g., mutations introduced by random or site-specific mutagenesis in vitro or by somatic mutation in vivo, or a conservative substitution to promote stability or manufacturing). However, the term “human antibody”, as used herein, is not intended to include antibodies in which CDR sequences derived from the germline of another mammalian species, such as a mouse, have been grafted onto human framework sequences.

Chimeric Antibody: The term “chimeric antibody” (or antigen-binding fragment thereof) is an antibody molecule (or antigen-binding fragment thereof) in which (a) the constant region, or a portion thereof, is altered, replaced or exchanged so that the antigen-binding site (variable region) is linked to a constant region of a different or altered class, effector function and/or species, or an entirely different molecule which confers new properties to the chimeric antibody, e.g., an enzyme, toxin, hormone, growth factor, drug, etc.; or (b) the variable region, or a portion thereof, is altered, replaced or exchanged with a variable region having a different or altered antigen specificity. For example, a mouse antibody can be modified by replacing its constant region with the constant region from a human immunoglobulin. Due to the replacement with a human constant region, the chimeric antibody can retain its specificity in recognizing the antigen while having reduced antigenicity in human as compared to the original mouse antibody.

Effector Function: The term “effector function” refers to an activity of an antibody molecule that is mediated by binding through a domain of the antibody other than the antigen-binding domain, usually mediated by binding of effector molecules. Effector function includes complement-mediated effector function, which is mediated by, for example, binding of the C1 component of the complement to the antibody. Activation of complement is important in the opsonization and lysis of cell pathogens. The activation of complement also stimulates the inflammatory response and may also be involved in autoimmune hypersensitivity. Effector function also includes Fc receptor (FcR)-mediated effector function, which may be triggered upon binding of the constant domain of an antibody to an Fc receptor (FcR). Binding of antibody to Fc receptors on cell surfaces triggers a number of important and diverse biological responses including engulfment and destruction of antibody-coated particles, clearance of immune complexes, lysis of antibody-coated target cells by killer cells (called antibody-dependent cell-mediated cytotoxicity, or ADCC), release of inflammatory mediators, placental transfer and control of immunoglobulin production. An effector function of an antibody may be altered by altering, e.g., enhancing or reducing, the affinity of the antibody for an effector molecule such as an Fc receptor or a complement component. Binding affinity will generally be varied by modifying the effector molecule binding site, and in this case it is appropriate to locate the site of interest and modify at least part of the site in a suitable way. It is also envisaged that an alteration in the binding site on the antibody for the effector molecule need not alter significantly the overall binding affinity but may alter the geometry of the interaction rendering the effector mechanism ineffective as in non-productive binding. It is further envisaged that an effector function may also be altered by modifying a site not directly involved in effector molecule binding, but otherwise involved in performance of the effector function.

Recognize: The term “recognize” as used herein refers to an ABM that finds and interacts (e.g., binds) with its epitope.

Epitope: An epitope, or antigenic determinant, is a portion of an antigen recognized by an antibody or other antigen-binding moiety as described herein. An epitope can be linear or conformational.

Nucleic Acid: The term “nucleic acid” is used herein interchangeably with the term “polynucleotide” and refers to deoxyribonucleotides or ribonucleotides and polymers thereof in either single- or double-stranded form. The term encompasses nucleic acids containing known nucleotide analogs or modified backbone residues or linkages, which are synthetic, naturally occurring, and non-naturally occurring, which have similar binding properties as the reference nucleic acid, and which are metabolized in a manner similar to the reference nucleotides. Examples of such analogs include, without limitation, phosphorothioates, phosphoramidates, methyl phosphonates, chiral-methyl phosphonates, 2-O-methyl ribonucleotides, peptide-nucleic acids (PNAs).

Unless otherwise indicated, a particular nucleic acid sequence also implicitly encompasses conservatively modified variants thereof (e.g., degenerate codon substitutions) and complementary sequences, as well as the sequence explicitly indicated. Specifically, as detailed below, degenerate codon substitutions may be achieved by generating sequences in which the third position of one or more selected (or all) codons is substituted with mixed-base and/or deoxyinosine residues (Batzer et al., (1991) Nucleic Acid Res. 19:5081; Ohtsuka et al., (1985) J. Biol. Chem. 260:2605-2608; and Rossolini et al., (1994) Mol. Cell. Probes 8:91-98).

Vector: The term “vector” is intended to refer to a polynucleotide molecule capable of transporting another polynucleotide to which it has been linked. One type of vector is a “plasmid”, which refers to a circular double stranded DNA loop into which additional DNA segments may be ligated. Another type of vector is a viral vector, where additional DNA segments may be ligated into the viral genome. Certain vectors are capable of autonomous replication in a host cell into which they are introduced (e.g., bacterial vectors having a bacterial origin of replication and episomal mammalian vectors). Other vectors (e.g., non-episomal mammalian vectors) can be integrated into the genome of a host cell upon introduction into the host cell, and thereby are replicated along with the host genome. Moreover, certain vectors are capable of directing the expression of genes to which they are operably linked. Such vectors are referred to herein as “recombinant expression vectors” (or simply, “expression vectors”). In general, expression vectors of utility in recombinant DNA techniques are often in the form of plasmids. In the present specification, “plasmid” and “vector” may be used interchangeably as the plasmid is the most commonly used form of vector. However, the disclosure is intended to include such other forms of expression vectors, such as viral vectors (e.g., replication defective retroviruses, adenoviruses and adeno-associated viruses), which serve equivalent functions.

Binding Sequences: In reference to Tables 8, 9, 10, 11, or 12 (including subparts thereof), the term “binding sequences” means an ABM having a full set of CDRs, a VH-VL pair, or an scFv set forth in that table.

VH-VL or VH-VL Pair: In reference to a VH-VL pair, whether on the same polypeptide chain or on different polypeptide chains, the terms “VH-VL” and “VH-VL pair” are used for convenience and are not intended to convey any particular orientation, unless the context dictates otherwise. Thus, a scFv comprising a “VH-VL” or “VH-VL pair” can have the VH and VL domains in any orientation, for example the VH N-terminal to the VL or the VL N-terminal to the VH.

Polypeptide and Protein: The terms “polypeptide” and “protein” are used interchangeably herein to refer to a polymer of amino acid residues. The phrases also apply to amino acid polymers in which one or more amino acid residue is an artificial chemical mimetic of a corresponding naturally occurring amino acid, as well as to naturally occurring amino acid polymers and non-naturally occurring amino acid polymer. Unless otherwise indicated, a particular polypeptide sequence also implicitly encompasses conservatively modified variants thereof.

Subject: The term “subject” includes human and non-human animals. Non-human animals include all vertebrates, e.g., mammals and non-mammals, such as non-human primates, sheep, dog, cow, chickens, amphibians, and reptiles. Except when noted, the terms “patient” or “subject” are used herein interchangeably.

Cancer: The term “cancer” refers to a disease characterized by the uncontrolled (and often rapid) growth of aberrant cells. Cancer cells can spread locally or through the bloodstream and lymphatic system to other parts of the body.

Tumor: The term “tumor” is used interchangeably with the term “cancer” herein, e.g., both terms encompass solid and liquid, e.g., diffuse or circulating, tumors. As used herein, the term “cancer” or “tumor” includes premalignant, as well as malignant cancers and tumors.

Tumor-Associated Antigen: As used herein, the term “tumor-associated antigen” or “TAA” refers to a molecule (typically a protein, carbohydrate, lipid or some combination thereof) that is expressed on the surface of a cancerous B cell, either entirely or as a fragment (e.g., MHC/peptide), and which is useful for the preferential targeting of a pharmacological agent to a cancerous B cell. As used herein, the term “cancerous B cell” refers to a B cell that is undergoing or has undergone uncontrolled proliferation. In some embodiments, a TAA is a marker expressed by both normal cells and cancer cells, e.g., a lineage marker, e.g., CD19 on B cells. In some embodiments, a TAA is a B cell surface molecule that is overexpressed in a cancerous B cell in comparison to a normal B cell, for instance, 1-fold over expression, 2-fold overexpression, 3-fold overexpression or more in comparison to a normal B cell. In some embodiments, a TAA is a cell surface molecule that is inappropriately synthesized in the cancerous B cell, for instance, a molecule that contains deletions, additions or mutations in comparison to the molecule expressed on a normal B cell. In some embodiments, a TAA will be expressed exclusively on the cell surface of a cancerous cell, entirely or as a fragment (e.g., MHC/peptide), and not synthesized or expressed on the surface of a normal cell. Accordingly, the term “TAA” encompasses B cell antigens that are specific to cancer cells, sometimes known in the art as tumor-specific antigens (“TSAs”).

B cell: As used herein, the term “B cell” refers to a cell of B cell lineage, which is a type of white blood cell of the lymphocyte subtype. Examples of B cells include plasmablasts, plasma cells, lymphoplasmacytoid cells, memory B cells, follicular B cells, marginal zone B cells, B-1 cells, B-2 cells, and regulatory B cells.

B cell malignancy: As used herein, a B cell malignancy refers to an uncontrolled proliferation of B cells. Examples of B cell malignancy include non-Hodgkin's lymphomas (NHL), Hodgkin's lymphomas, leukemia, and myeloma. For example, a B cell malignancy can be, but is not limited to, multiple myeloma, chronic lymphocytic leukemia (CLL)/small lymphocytic lymphoma (SLL), follicular lymphoma, mantle cell lymphoma (MCL), diffuse large B-cell lymphoma (DLBCL), marginal zone lymphomas, Burkitt lymphoma, lymphoplasmacytic lymphoma (Waldenstrom macroglobulinemia), hairy cell leukemia, primary central nervous system (CNS) lymphoma, primary mediastinal large B-cell lymphoma, mediastinal grey-zone lymphoma (MGZL), splenic marginal zone B-cell lymphoma, extranodal marginal zone B-cell lymphoma of MALT, nodal marginal zone B-cell lymphoma, and primary effusion lymphoma, and plasmacytic dendritic cell neoplasms.

Treat, Treatment, Treating: As used herein, the terms “treat”, “treatment” and “treating” refer to the reduction or amelioration of the progression, severity and/or duration of a proliferative disorder, or the amelioration of one or more symptoms (e.g., one or more discernible symptoms) of a proliferative disorder resulting from the administration of one or more MBMs (e.g., TBMs) of the disclosure. In specific embodiments, the terms “treat”, “treatment” and “treating” refer to the amelioration of at least one measurable physical parameter of a proliferative disorder, such as growth of a tumor, not necessarily discernible by the patient. In other embodiments the terms “treat”, “treatment” and “treating” refer to the inhibition of the progression of a proliferative disorder, either physically by, e.g., stabilization of a discernible symptom, physiologically by, e.g., stabilization of a physical parameter, or both. In other embodiments the terms “treat”, “treatment” and “treating” refer to the reduction or stabilization of tumor size or cancerous cell count.

### 7.2. Antigen Binding Modules

Typically, one or more ABMs of the MBMs of the disclosure comprise immunoglobulin-based antigen-binding domains, for example the sequences of antibody fragments or derivatives. These antibody fragments and derivatives typically include the CDRs of an antibody and can include larger fragments and derivatives thereof, e.g., Fabs, scFabs, Fvs, and scFvs.

Immunoglobulin-based ABMs can comprise modifications to framework residues within a VH and/or a VL, e.g. to improve the properties of a MBM containing the ABM. For example, framework modifications can be made to decrease immunogenicity of a MBM. One approach for making such framework modifications is to “back-mutate” one or more framework residues of the ABM to a corresponding germline sequence. Such residues can be identified by comparing framework sequences to germline sequences from which the ABM is derived. To “match” framework region sequences to desired germline configuration, residues can be “back-mutated” to a corresponding germline sequence by, for example, site-directed mutagenesis. MBMs having such “back-mutated” ABMs are intended to be encompassed by the disclosure.

Another type of framework modification involves mutating one or more residues within a framework region, or even within one or more CDR regions, to remove T-cell epitopes to thereby reduce potential immunogenicity of a MBM. This approach is also referred to as “deimmunization” and is described in further detail in U.S. Patent Publication No. 20030153043 by Carr et al.

ABMs can also be modified to have altered glycosylation, which can be useful, for example, to increase the affinity of a MBM for one or more of its antigens. Such carbohydrate modifications can be accomplished by, for example, altering one or more sites of glycosylation within a ABM sequence. For example, one or more amino acid substitutions can be made that result in elimination of one or more variable region framework glycosylation sites to thereby eliminate glycosylation at that site. Such aglycosylation may increase the affinity of the MBM for an antigen. Such an approach is described in, e.g., U.S. Pat. Nos. 5,714,350 and 6,350,861 by Co et al.

### 7.2.1. Immunoglobulin Based Modules

### 7.2.1.1. Fabs

In certain aspects, an ABM of the disclosure is a Fab domain. Fab domains can be produced by proteolytic cleavage of immunoglobulin molecules, using enzymes such as papain, or through recombinant expression. Fab domains typically comprise a CH1 domain attached to a VH domain which pairs with a CL domain attached to a VL domain.

In a wild-type immunoglobulin, the VH domain is paired with the VL domain to constitute the Fv region, and the CHC domain is paired with the CL domain to further stabilize the binding module. A disulfide bond between the two constant domains can further stabilize the Fab domain.

For the MBMs (e.g., TBMs) of the disclosure, it is advantageous to use Fab heterodimerization strategies to permit the correct association of Fab domains belonging to the same ABM and minimize aberrant pairing of Fab domains belonging to different ABMs. For example, the Fab heterodimerization strategies shown in Table 1 below can be used:

TABLE 1

| | Fab Heterodimerization Strategies | | | | | |
|---|---|---|---|---|---|---|
| Name | STRATEGY | VH | CH1 | VL | CL | REFERENCE |
| F1 | CrossMabCH1- | WT | CL domain | WT | CH1 domain | Schaefer et al., 2011, |
| | CL | | | | | Cancer Cell 2011; |
| | | | | | | 20: 472-86; |
| | | | | | | PMID: 22014573. |
| F2 | orthogonal Fab | 39K, 62E | H172A, | 1R, 38D, | L135Y, | Lewis et al., 2014, Nat |
| | VHVRD1CH1CRD2- | | F174G | (36F) | S176W | Biotechnol 32: 191-8 |
| | VLVRD1CλCRD2 | | | | | |
| F3 | orthogonal Fab | 39Y | WT | 38R | WT | Lewis etal., 2014, Nat |
| | VHVRD2CH1wt- | | | | | Biotechnol 32: 191-8 |
| | VLVRD2Cλwt | | | | | |
| F4 | TCR CαCβ | 39K | TCR Cα | 38D | TCR Cβ | Wu et al., 2015, MAbs |
| | | | | | | 7: 364-76 |
| F5 | CR3 | WT | T192E | WT | N137K, | Golay at al., 2016, J |
| | | | | | S114A | Immunol 196: 3199- |
| | | | | | | 211. |
| F6 | MUT4 | WT | L143Q, | WT | V133T, | Golay at al., 2016, J |
| | | | S188V | | S176V | Immunol 196: 3199- |
| | | | | | | 211. |
| F7 | DuetMab | WT | F126C | WT | S121C | Mazor et al., 2015, |
| | | | | | | MAbs 7: 377-89; Mazor |
| | | | | | | et al., 2015, MAbs |
| | | | | | | 7: 461-669. |

Accordingly, in certain embodiments, correct association between the two polypeptides of a Fab is promoted by exchanging the VL and VH domains of the Fab for each other or exchanging the CH1 and CL domains for each other, e.g., as described in WO 2009/080251.

Correct Fab pairing can also be promoted by introducing one or more amino acid modifications in the CH1 domain and one or more amino acid modifications in the CL domain of the Fab and/or one or more amino acid modifications in the VH domain and one or more amino acid modifications in the VL domain. The amino acids that are modified are typically part of the VH:VL and CH1:CL interface such that the Fab components preferentially pair with each other rather than with components of other Fabs.

In one embodiment, the one or amino acid modifications are limited to the conserved framework residues of the variable (VH, VL) and constant (CH1, CL) domains as indicated by the Kabat numbering of residues. Almagro, 2008, Frontiers In Bioscience 13:1619-1633 provides a definition of the framework residues on the basis of Kabat, Chothia, and IMGT numbering schemes.

In one embodiment, the modifications introduced in the VH and CH1 and/or VL and CL domains are complementary to each other. Complementarity at the heavy and light chain interface can be achieved on the basis of steric and hydrophobic contacts, electrostatic/charge interactions or a combination of the variety of interactions. The complementarity between protein surfaces is broadly described in the literature in terms of lock and key fit, knob into hole, protrusion and cavity, donor and acceptor etc., all implying the nature of structural and chemical match between the two interacting surfaces.

In one embodiment, the one or more introduced modifications introduce a new hydrogen bond across the interface of the Fab components. In one embodiment, the one or more introduced modifications introduce a new salt bridge across the interface of the Fab components. Exemplary substitutions are described in WO 2014/150973 and WO 2014/082179.

In some embodiments, the Fab domain comprises a 192E substitution in the CH1 domain and 114A and 137K substitutions in the CL domain, which introduces a salt-bridge between the CH1 and CL domains (see, Golay et al., 2016, J Immunol 196:3199-211).

In some embodiments, the Fab domain comprises a 143Q and 188V substitutions in the CH1 domain and 113T and 176V substitutions in the CL domain, which serves to swap hydrophobic and polar regions of contact between the CH1 and CL domain (see, Golay et al., 2016, J Immunol 196:3199-211).

In some embodiments, the Fab domain can comprise modifications in some or all of the VH, CH1, VL, CL domains to introduce orthogonal Fab interfaces which promote correct assembly of Fab domains (Lewis et al., 2014 Nature Biotechnology 32:191-198). In an embodiment, 39K, 62E modifications are introduced in the VH domain, H172A, F174G modifications are introduced in the CH1 domain, 1R, 38D, (36F) modifications are introduced in the VL domain, and L135Y, S176W modifications are introduced in the CL domain. In another embodiment, a 39Y modification is introduced in the VH domain and a 38R modification is introduced in the VL domain.

Fab domains can also be modified to replace the native CH1:CL disulfide bond with an engineered disulfide bond, thereby increasing the efficiency of Fab component pairing. For example, an engineered disulfide bond can be introduced by introducing a 126C in the CH1 domain and a 121C in the CL domain (see, Mazor et al., 2015, MAbs 7:377-89).

Fab domains can also be modified by replacing the CH1 domain and CL domain with alternative domains that promote correct assembly. For example, Wu et al., 2015, MAbs 7:364-76, describes substituting the CH1 domain with the constant domain of the α T cell receptor and substituting the CL domain with the β domain of the T cell receptor, and pairing these domain replacements with an additional charge-charge interaction between the VL and VH domains by introducing a 38D modification in the VL domain and a 39K modification in the VH domain.

ABMs of the disclosure can comprise a single chain Fab fragment, which is a polypeptide consisting of an antibody heavy chain variable domain (VH), an antibody constant domain 1 (CH1), an antibody light chain variable domain (VL), an antibody light chain constant domain (CL) and a linker. In some embodiments, the antibody domains and the linker have one of the following orders in N-terminal to C-terminal direction: a) VH—CH1-linker-VL-CL, b) VL-CL-linker-VH—CH1, c) VH—CL-linker-VL-CH1 or d) VL-CH1-linker-VH—CL. The linker can be a polypeptide of at least 30 amino acids, e.g., between 32 and 50 amino acids. The single chain Fab domains are stabilized via the natural disulfide bond between the CL domain and the CH1 domain.

In an embodiment, the antibody domains and the linker in the single chain Fab fragment have one of the following orders in N-terminal to C-terminal direction: a) VH—CH1-linker-VL-CL, or b) VL-CL-linker-VH—CH1. In some cases, VL-CL-linker-VH—CH1 is used.

In another embodiment, the antibody domains and the linker in the single chain Fab fragment have one of the following orders in N-terminal to C-terminal direction: a) VH—CL-linker-VL-CH1 or b) VL-CH1-linker-VH—CL.

Optionally in the single chain Fab fragment, additionally to the natural disulfide bond between the CL-domain and the CH1 domain, also the antibody heavy chain variable domain (VH) and the antibody light chain variable domain (VL) are disulfide stabilized by introduction of a disulfide bond between the following positions: i) heavy chain variable domain position 44 to light chain variable domain position 100, ii) heavy chain variable domain position 105 to light chain variable domain position 43, or iii) heavy chain variable domain position 101 to light chain variable domain position 100 (numbering according to EU index of Kabat).

Such further disulfide stabilization of single chain Fab fragments is achieved by the introduction of a disulfide bond between the variable domains VH and VL of the single chain Fab fragments. Techniques to introduce unnatural disulfide bridges for stabilization for a single chain Fv are described e.g. in WO 94/029350, Rajagopal et al., 1997, Prot. Engin. 10:1453-59; Kobayashi et al., 1998, Nuclear Medicine & Biology, 25:387-393; and Schmidt, et al., 1999, Oncogene 18:1711-1721. In one embodiment, the optional disulfide bond between the variable domains of the single chain Fab fragments is between heavy chain variable domain position 44 and light chain variable domain position 100. In one embodiment, the optional disulfide bond between the variable domains of the single chain Fab fragments is between heavy chain variable domain position 105 and light chain variable domain position 43 (numbering according to EU index of Kabat).

### 7.2.1.2. scFvs

Single chain Fv or “scFv” antibody fragments comprise the VH and VL domains of an antibody in a single polypeptide chain, are capable of being expressed as a single chain polypeptide, and retain the specificity of the intact antibody from which it is derived. Generally, the scFv polypeptide further comprises a polypeptide linker between the VH and VL domain that enables the scFv to form the desired structure for target binding. Examples of linkers suitable for connecting the VH and VL chains of an scFV are the ABM linkers identified in Section 7.3.3, for example any of the linkers designated L1 through L54.

Unless specified, as used herein an scFv may have the VL and VH variable regions in either order, e.g., with respect to the N-terminal and C-terminal ends of the polypeptide, the scFv may comprise VL-linker-VH or may comprise VH-linker-VL.

To create an scFv-encoding nucleic acid, the VH and VL-encoding DNA fragments are operably linked to another fragment encoding a linker, e.g., encoding any of the ABM linkers described in Section 7.3.3 (such as the amino acid sequence (Gly4^{~}Ser)3 (SEQ ID NO: 724)), such that the VH and VL sequences can be expressed as a contiguous single-chain protein, with the VL and VH regions joined by the flexible linker (see e.g., Bird et al., 1988, Science 242:423-426; Huston et al., 1988, Proc. Natl. Acad. Sci. USA 85:5879-5883; McCafferty et al., 1990, Nature 348:552-554).

### 7.2.1.3. Other Immunoglobulin-Based Modules

MBMs of the disclosure can also comprise ABMs having an immunoglobulin format which is other than Fab or scFv, for example Fv, dsFv, (Fab′)2, a single domain antibody (SDAB), a VH or VL domain, or a camelid VHH domain (also called a nanobody).

An ABM can be a single domain antibody composed of a single VH or VL domain which exhibits sufficient affinity to the target. In a specific embodiment, the single domain antibody is a camelid VHH domain (see, e.g., Riechmann, 1999, Journal of Immunological Methods 231:25-38; WO 94/04678).

### 7.2.2. Non-Immunoglobulin Based Modules

In certain embodiments, one or more of the ABMs of the disclosure are derived from non-antibody scaffold proteins (including, but not limited to, designed ankyrin repeat proteins (DARPins), Avimers (short for avidity multimers), Anticalin/Lipocalins, Centyrins, Kunitz domains, Adnexins, Affilins, Affitins (also known as Nonfitins), Knottins, Pronectins, Versabodies, Duocalins, and Fynomers), ligands, receptors, cytokines or chemokines.

Non-immunoglobulin scaffolds that can be used in the MBMs of the disclosure include those listed in Tables 3 and 4 of Mintz and Crea, 2013, Bioprocess International 11(2):40-48; in FIG. 1, Table 1 and Figure I of Vazquez-Lombardi et al., 2015, Drug Discovery Today 20(10):1271-83; in Table 1 and Box 2 of Skrlec et al., 2015, Trends in Biotechnology 33(7):408-18. The contents of Tables 3 and 4 of Mintz and Crea, 2013, Bioprocess International 11(2):40-48; in FIG. 1, Table 1 and Figure I of Vazquez-Lombardi et al., 2015, Drug Discovery Today 20(10):1271-83; in Table 1 and Box 2 of Skrlec et al., 2015, Trends in Biotechnology 33(7):408-18 (collectively, “Scaffold Disclosures”). In a particular embodiment, the Scaffold Disclosures are incorporated by reference for what they disclose relating to Adnexins. In another embodiment, the Scaffold Disclosures are incorporated by reference for what they disclose relating to Avimers. In another embodiment, the Scaffold Disclosures are incorporated by reference for what they disclose relating to Affibodies. In yet another embodiment, the Scaffold Disclosures are incorporated by reference for what they disclose relating to Anticalins. In yet another embodiment, the Scaffold Disclosures are incorporated by reference for what they disclose relating to DARPins. In yet another embodiment, the Scaffold Disclosures are incorporated by reference for what they disclose relating to Kunitz domains. In yet another embodiment, the Scaffold Disclosures are incorporated by reference for what they disclose relating to Knottins. In yet another embodiment, the Scaffold Disclosures are incorporated by reference for what they disclose relating to Pronectins. In yet another embodiment, the Scaffold Disclosures are incorporated by reference for what they disclose relating to Nanofitins. In yet another embodiment, the Scaffold Disclosures are incorporated by reference for what they disclose relating to Affilins. In yet another embodiment, the Scaffold Disclosures are incorporated by reference for what they disclose relating to Adnectins. In yet another embodiment, the Scaffold Disclosures are incorporated by reference for what they disclose relating to ABDs. In yet another embodiment, the Scaffold Disclosures are incorporated by reference for what they disclose relating to Adhirons. In yet another embodiment, the Scaffold Disclosures are incorporated by reference for what they disclose relating to Affimers. In yet another embodiment, the Scaffold Disclosures are incorporated by reference for what they disclose relating to Alphabodies. In yet another embodiment, the Scaffold Disclosures are incorporated by reference for what they disclose relating to Armadillo Repeat Proteins. In yet another embodiment, the Scaffold Disclosures are incorporated by reference for what they disclose relating to Atrimers/Tetranectins. In yet another embodiment, the Scaffold Disclosures are incorporated by reference for what they disclose relating to Obodies/OB-folds. In yet another embodiment, the Scaffold Disclosures are incorporated by reference for what they disclose relating to Centyrins. In yet another embodiment, the Scaffold Disclosures are incorporated by reference for what they disclose relating to Repebodies. In yet another embodiment, the Scaffold Disclosures are incorporated by reference for what they disclose relating to Anticalins. In yet another embodiment, the Scaffold Disclosures are incorporated by reference for what they disclose relating to Atrimers. In yet another embodiment, the Scaffold Disclosures are incorporated by reference for what they disclose relating to bicyclic peptides. In yet another embodiment, the Scaffold Disclosures are incorporated by reference for what they disclose relating to cys-knots. In yet another embodiment, the Scaffold Disclosures are incorporated by reference for what they disclose relating to Fn3 scaffolds (including Adnectins, Centryrins, Pronectins, and Tn3).

In an embodiment, an ABM of the disclosure can be a designed ankyrin repeat protein (“DARPin”). DARPins are antibody mimetic proteins that typically exhibit highly specific and high-affinity target protein binding. They are typically genetically engineered and derived from natural ankyrin proteins and consist of at least three, usually four or five repeat motifs of these proteins. Their molecular mass is about 14 or 18 kDa (kilodaltons) for four- or five-repeat DARPins, respectively. Examples of DARPins can be found, for example in U.S. Pat. No. 7,417,130. Multispecific binding molecules comprising DARPin binding modules and immunoglobulin-based binding modules are disclosed in, for example, U.S. Publication No. 2015/0030596 A1.

In another embodiment, an ABM of the disclosure can be an Affibody. An Affibody is well known in the art and refers to affinity proteins based on a 58 amino acid residue protein domain, derived from one of the IgG binding domain of staphylococcal protein A.

In another embodiment, an ABM of the disclosure can be an Anticalin. Anticalins are well known in the art and refer to another antibody mimetic technology, where the binding specificity is derived from Lipocalins. Anticalins may also be formatted as dual targeting protein, called Duocalins.

In another embodiment, an ABM of the disclosure can be a Versabody. Versabodies are well known in the art and refer to another antibody mimetic technology. They are small proteins of 3-5 kDa with >15% cysteines, which form a high disulfide density scaffold, replacing the hydrophobic core of typical proteins.

Other non-immunoglobulin ABMs include “A” domain oligomers (also known as Avimers) (see for example, U.S. Patent Application Publication Nos. 2005/0164301, 2005/0048512, and 2004/017576), Fn3 based protein scaffolds (see for example, U.S. Patent Application Publication 2003/0170753), VASP polypeptides, Avian pancreatic polypeptide (aPP), Tetranectin (based on CTLD3), Affililin (based on γB-crystallin/ubiquitin), Knottins, SH3 domains, PDZ domains, Tendamistat, Neocarzinostatin, Protein A domains, Lipocalins, Transferrin, or Kunitz domains. In one aspect, ABMs useful in the construction of the MBMs of the disclosure comprise fibronectin-based scaffolds as exemplified in WO 2011/130324.

### 7.3. Connectors

It is contemplated that the MBMs of the disclosure can in some instances include pairs of ABMs or ABM chains (e.g., the VH—CH1 or VL-CL component of a Fab) connected directly to one another, e.g., as a fusion protein without a linker. For example, the MBMs of the disclosure comprise connector moieties linking individual ABMs or ABM chains. The use of connector moieties can improve target binding, for example by increasing flexibility of the ABMs within a MBM and thus reducing steric hindrance. The ABMs can be connected to one another through, for example, Fc domains (each Fc domain representing a pair of associated Fc regions) and/or ABM linkers. The use of Fc domains will typically require the use of hinge regions as connectors of the ABMs or ABM chains for optimal antigen binding. Thus, the term “connector” encompasses, but is not limited to, Fc regions, Fc domains, hinge regions, and ABM linkers.

Connectors can be selected or modified to, for example, increase or decrease the biological half-life of a MBM of the disclosure. For example, to decrease biological half-life, one or more amino acid mutations can be introduced into a CH2-CH3 domain interface region of a Fc-hinge fragment such that a MBM comprising the fragment has impaired Staphylococcyl Protein A (SpA) binding relative to native Fc-hinge domain SpA binding. This approach is described in further detail in U.S. Pat. No. 6,165,745 by Ward et al. Alternatively, a MBM can be modified to increase its biological half-life. For example, one or more of the following mutations can be introduced: T252L, T254S, T256F, as described in U.S. Pat. No. 6,277,375 to Ward. Alternatively, to increase the biological half-life, a MBM can be altered within a CH1 or CL region to contain a salvage receptor binding epitope taken from two loops of a CH2 domain of an Fc region of an IgG, as described in U.S. Pat. Nos. 5,869,046 and 6,121,022 by Presta et al.

Examples of Fc domains (formed by the pairing of two Fc regions), hinge regions and ABM linkers are described in Sections 7.3.1, 7.3.2, and 7.3.3, respectively.

### 7.3.1. Fc Domains

The MBMs (e.g., TBMs) of the disclosure can include an Fc domain derived from any suitable species. In one embodiment the Fc domain is derived from a human Fc domain.

The Fc domain may be derived from any suitable class of antibody, including IgA (including subclasses IgA1 and IgA2), IgD, IgE, IgG (including subclasses IgG1, IgG2, IgG3 and IgG4), and IgM. In one embodiment, the Fc domain is derived from IgG1, IgG2, IgG3 or IgG4. In one embodiment the Fc domain is derived from IgG1. In one embodiment the Fc domain is derived from IgG4.

The Fc domain comprises two polypeptide chains, each referred to as a heavy chain Fc region. The two heavy chain Fc regions dimerize to create the Fc domain. The two Fc regions within the Fc domain may be the same or different from one another. In a native antibody the Fc regions are typically identical, but for the purpose of producing multispecific binding molecules, e.g., the TBMs of the disclosure, the Fc regions might advantageously be different to allow for heterodimerization, as described in Section 7.3.1.5 below.

Typically each heavy chain Fc region comprises or consists of two or three heavy chain constant domains.

In native antibodies, the heavy chain Fc region of IgA, IgD and IgG is composed of two heavy chain constant domains (CH2 and CH3) and that of IgE and IgM is composed of three heavy chain constant domains (CH2, CH3 and CH4). These dimerize to create an Fc domain.

In the present disclosure, the heavy chain Fc region can comprise heavy chain constant domains from one or more different classes of antibody, for example one, two or three different classes.

In one embodiment the heavy chain Fc region comprises CH2 and CH3 domains derived from IgG1.

In one embodiment the heavy chain Fc region comprises CH2 and CH3 domains derived from IgG2.

In one embodiment the heavy chain Fc region comprises CH2 and CH3 domains derived from IgG3.

In one embodiment the heavy chain Fc region comprises CH2 and CH3 domains derived from IgG4.

In one embodiment the heavy chain Fc region comprises a CH4 domain from IgM. The IgM CH4 domain is typically located at the C-terminus of the CH3 domain.

In one embodiment the heavy chain Fc region comprises CH2 and CH3 domains derived from IgG and a CH4 domain derived from IgM.

It will be appreciated that the heavy chain constant domains for use in producing a heavy chain Fc region for the MBMs of the present disclosure may include variants of the naturally occurring constant domains described above. Such variants may comprise one or more amino acid variations compared to wild type constant domains. In one example the heavy chain Fc region of the present disclosure comprises at least one constant domain that varies in sequence from the wild type constant domain. It will be appreciated that the variant constant domains may be longer or shorter than the wild type constant domain. For example, the variant constant domains are at least 60% identical or similar to a wild type constant domain. In another example the variant constant domains are at least 70% identical or similar. In another example the variant constant domains are at least 75% identical or similar. In another example the variant constant domains are at least 80% identical or similar. In another example the variant constant domains are at least 85% identical or similar. In another example the variant constant domains are at least 90% identical or similar. In another example the variant constant domains are at least 95% identical or similar. In another example the variant constant domains are at least 99% identical or similar. Exemplary Fc variants are described in Sections 7.3.1.1 through 7.3.1.5, infra.

IgM and IgA occur naturally in humans as covalent multimers of the common H2L2 antibody unit. IgM occurs as a pentamer when it has incorporated a J-chain, or as a hexamer when it lacks a J-chain. IgA occurs as monomer and dimer forms. The heavy chains of IgM and IgA possess an 18 amino acid extension to the C-terminal constant domain, known as a tailpiece. The tailpiece includes a cysteine residue that forms a disulfide bond between heavy chains in the polymer, and is believed to have an important role in polymerization. The tailpiece also contains a glycosylation site. In certain embodiments, the MBMs of the present disclosure do not comprise a tailpiece.

The Fc domains that are incorporated into the MBMs (e.g., TBMs) of the present disclosure may comprise one or more modifications that alter one or more functional properties of the proteins, such as serum half-life, complement fixation, Fc receptor binding, and/or antigen-dependent cellular cytotoxicity. Furthermore, a MBM of the disclosure can be chemically modified (e.g., one or more chemical moieties can be attached to the MBM) or be modified to alter its glycosylation, again to alter one or more functional properties of the MBM.

Effector function of an antibody molecule includes complement-mediated effector function, which is mediated by, for example, binding of the C1 component of the complement to the antibody. Activation of complement is important in the opsonization and direct lysis of pathogens. In addition, it stimulates the inflammatory response by recruiting and activating phagocytes to the site of complement activation. Effector function includes Fc receptor (FcR)-mediated effector function, which may be triggered upon binding of the constant domains of an antibody to an Fc receptor (FcR). Antigen-antibody complex-mediated crosslinking of Fc receptors on effector cell surfaces triggers a number of important and diverse biological responses including engulfment and destruction of antibody-coated particles, clearance of immune complexes, lysis of antibody-coated target cells by killer cells (called antibody-dependent cell-mediated cytotoxicity, or ADCC), release of inflammatory mediators, placental transfer and control of immunoglobulin production.

Fc regions can be altered by replacing at least one amino acid residue with a different amino acid residue to alter the effector functions. For example, one or more amino acids can be replaced with a different amino acid residue such that the Fc region has an altered affinity for an effector ligand. The effector ligand to which affinity is altered can be, for example, an Fc receptor or the C1 component of complement. This approach is described in, e.g., U.S. Pat. Nos. 5,624,821 and 5,648,260, both by Winter et al. Modified Fc regions can also alter Clq binding and/or reduce or abolish complement dependent cytotoxicity (CDC). This approach is described in, e.g., U.S. Pat. Nos. 6,194,551 by Idusogie et al. Modified Fc regions can also alter the ability of a Fc region to fix complement. This approach is described in, e.g., the PCT Publication WO 94/29351 by Bodmer et al. Allotypic amino acid residues include, but are not limited to, constant region of a heavy chain of the IgG1, IgG2, and IgG3 subclasses as well as constant region of a light chain of the kappa isotype as described by Jefferis et al., 2009, MAbs, 1:332-338.

Fc regions can also be modified to “silence” the effector function, for example, to reduce or eliminate the ability of a MBM to mediate antibody dependent cellular cytotoxicity (ADCC) and/or antibody dependent cellular phagocytosis (ADCP). This can be achieved, for example, by introducing a mutation in an Fc region. Such mutations have been described in the art: LALA and N297A (Strohl, 2009, Curr. Opin. Biotechnol. 20(6):685-691); and D265A (Baudino et al., 2008, J. Immunol. 181: 6664-69; Strohl, supra). Examples of silent Fc IgG1 antibodies comprise the so-called LALA mutant comprising L234A and L235A mutation in the IgG1 Fc amino acid sequence. Another example of a silent IgG1 antibody comprises the D265A mutation. Another silent IgG1 antibody comprises the so-called DAPA mutant comprising D265A and P329A mutations in the IgG1 Fc amino acid sequence. Another silent IgG1 antibody comprises the N297A mutation, which results in aglycosylated/non-glycosylated antibodies.

Fc regions can be modified to increase the ability of a MBM containing the Fc region to mediate antibody dependent cellular cytotoxicity (ADCC) and/or antibody dependent cellular phagocytosis (ADCP), for example, by modifying one or more amino acid residues to increase the affinity of the MBM for an activating Fcγ receptor, or to decrease the affinity of the MBM for an inhibitory Fcγ receptor. Human activating Fcγ receptors include FcγRIa, FcγRIIa, FcγRIIIa, and FcγRIIIb, and human inhibitory Fcγ receptor includes FcγRIIb. This approach is described in, e.g., the PCT Publication WO 00/42072 by Presta. Moreover, binding sites on human IgG1 for FcγRI, FcγRII, FcγRIII and FcRn have been mapped and variants with improved binding have been described (see Shields et al., J. Biol. Chem. 276:6591-6604, 2001). Optimization of Fc-mediated effector functions of monoclonal antibodies such as increased ADCC/ADCP function has been described (see Strohl, 2009, Current Opinion in Biotechnology 20:685-691). Mutations that can enhance ADCC/ADCP function include one or more mutations selected from G236A, S239D, F243L, P247I, D280H, K290S, R292P, S298A, S298D, S298V, Y300L, V3051, A330L, 1332E, E333A, K334A, A339D, A339Q, A339T, and P396L (all positions by EU numbering).

Fc regions can also be modified to increase the ability of a MBM to mediate ADCC and/or ADCP, for example, by modifying one or more amino acids to increase the affinity of the MBM for an activating receptor that would typically not recognize the parent MBM, such as FcaRI. This approach is described in, e.g., Borrok et al., 2015, mAbs. 7(4):743-751.

Accordingly, in certain aspects, the MBMs of the present disclosure may include Fc domains with altered effector function such as, but not limited, binding to Fc-receptors such as FcRn or leukocyte receptors (for example, as described above or in Section 7.3.1.1), binding to complement (for example as described above or in Section 7.3.1.2), modified disulfide bond architecture (for example as described above or in Section 7.3.1.3), or altered glycosylation patterns (for example as described above or in Section 7.3.1.4). The Fc domains can also be altered to include modifications that improve manufacturability of asymmetric MBMs, for example by allowing heterodimerization, which is the preferential pairing of non-identical Fc regions over identical Fc regions. Heterodimerization permits the production of MBMs in which different ABMs are connected to one another by an Fc domain containing Fc regions that differ in sequence. Examples of heterodimerization strategies are exemplified in Section 7.3.1.5 (and subsections thereof).

It will be appreciated that any of the modifications described in Sections 7.3.1.1 through 7.3.1.5 can be combined in any suitable manner to achieve the desired functional properties and/or combined with other modifications to alter the properties of the MBMs.

### 7.3.1.1. Fc Domains with Altered FcR Binding

The Fc domains of the MBMs (e.g., TBMs) of the disclosure may show altered binding to one or more Fc-receptors (FcRs) in comparison with the corresponding native immunoglobulin. The binding to any particular Fc-receptor may be increased or decreased. In one embodiment, the Fc domain comprises one or more modifications which alter its Fc-receptor binding profile.

Human cells can express a number of membrane bound FcRs selected from FcaR, FcεR, FcγR, FcRn and glycan receptors. Some cells are also capable of expressing soluble (ectodomain) FcR (Fridman et al., 1993, J Leukocyte Biology 54: 504-512). FcγR can be further divided by affinity of IgG binding (high/low) and biological effect (activating/inhibiting). Human FcγRI is widely considered to be the sole ‘high affinity’ receptor whilst all of the others are considered as medium to low. FcγRIIb is the sole receptor with ‘inhibitory’ functionality by virtue of its intracellular ITIM motif whilst all of the others are considered as ‘activating’ by virtue of ITAM motifs or pairing with the common FcγR-γchain. FcγRIIIb is also unique in that although activatory it associates with the cell via a GPI anchor. In total, humans express six “standard” FcγRs: FcγRI, FcγRIIa, FcγRIIb, FcγRIIc, FcγRIIIa, and FcγRIIIb. In addition to these sequences there are a large number of sequence or allotypic variants spread across these families. Some of these have been found to have important functional consequence and so are sometimes considered to be receptor sub-types of their own. Examples include FcγRIIa^{H134R}, FcγRIIb^{I190T}, FcγRIIIa^{F158V}, FcγRIIIb^{NA1}, FcγRIIIb^{NA2}, and FcγRIII^{SH}. Each receptor sequence has been shown to have different affinities for the 4 sub-classes of IgG: IgG1, IgG2, IgG3 and IgG4 (Bruhns, 1993, Blood 113:3716-3725). Other species have somewhat different numbers and functionality of FcγR, with the mouse system being the best studied to date and comprising of 4 FcγR, FcγRI FcγRIIb FcγRIII FcγRIV (Bruhns, 2012, Blood 119:5640-5649). Human FcγRI on cells is normally considered to be ‘occupied’ by monomeric IgG in normal serum conditions due to its affinity for IgG1/IgG3/IgG4 (about 10⁻⁸ M) and the concentration of these IgG in serum (about 10 mg/ml). Hence cells bearing FcγRI on their surface are considered to be capable for “screening” or “sampling” of their antigenic environment vicariously through the bound polyspecific IgG. The other receptors having lower affinities for IgG sub-classes (in the range of about 10⁻⁵-10⁻⁷ M) are normally considered to be “unoccupied.” The low affinity receptors are hence inherently sensitive to the detection of and activation by antibody involved immune complexes. The increased Fc density in an antibody immune complex results in increased functional affinity of binding avidity to low affinity FcγR. This has been demonstrated in vitro using a number of methods (Shields et al., 2001, J Biol Chem 276(9):6591-6604; Lux et al., 2013, J Immunol 190:4315-4323). It has also been implicated as being one of the primary modes of action in the use of anti-RhD to treat ITP in humans (Crow, 2008, Transfusion Medicine Reviews 22:103-116).

Many cell types express multiple types of FcγR and so binding of IgG or antibody immune complex to cells bearing FcγR can have multiple and complex outcomes depending upon the biological context. Most simply, cells can either receive an activatory, inhibitory or mixed signal. This can result in events such as phagocytosis (e.g., macrophages and neutrophils), antigen processing (e.g., dendritic cells), reduced IgG production (e.g., B-cells) or degranulation (e.g., neutrophils, mast cells). There are data to support that the inhibitory signal from FcγRIIb can dominate that of activatory signals (Proulx, 2010, Clinical Immunology 135:422-429).

FcRn has a crucial role in maintaining the long half-life of IgG in the serum of adults and children. The receptor binds IgG in acidified vesicles (pH<6.5) protecting the IgG molecule from degradation, and then releasing it at the higher pH of 7.4 in blood.

FcRn is unlike leukocyte Fc receptors, and instead, has structural similarity to MHC class I molecules. It is a heterodimer composed of a β₂-microglobulin chain, non-covalently attached to a membrane-bound chain that includes three extracellular domains. One of these domains, including a carbohydrate chain, together with β₂-microglobulin interacts with a site between the CH2 and CH3 domains of Fc. The interaction includes salt bridges made to histidine residues on IgG that are positively charged at pH<6.5. At higher pH, the His residues lose their positive charges, the FcRn-IgG interaction is weakened and IgG dissociates.

In one embodiment, a MBM of the disclosure comprises an Fc domain that binds to human FcRn.

In one embodiment, the Fc domain has an (e.g., one or two) Fc regions comprising a histidine residue at position 310, and in some cases also at position 435. These histidine residues are important for human FcRn binding. In one embodiment, the histidine residues at positions 310 and 435 are native residues, i.e., positions 310 and 435 are not modified. Alternatively, one or both of these histidine residues may be present as a result of a modification.

The MBMs of the disclosure may comprise one or more Fc regions that alter Fc binding to FcRn. The altered binding may be increased binding or decreased binding.

In one embodiment, the MBM comprises an Fc domain in which at least one (and optionally both) Fc regions comprises one or more modifications such that it binds to FcRn with greater affinity and avidity than the corresponding native immunoglobulin.

In one embodiment, the Fc region is modified by substituting the threonine residue at position 250 with a glutamine residue (T250Q).

In one embodiment, the Fc region is modified by substituting the methionine residue at position 252 with a tyrosine residue (M252Y)

In one embodiment, the Fc region is modified by substituting the serine residue at position 254 with a threonine residue (S254T).

In one embodiment, the Fc region is modified by substituting the threonine residue at position 256 with a glutamic acid residue (T256E).

In one embodiment, the Fc region is modified by substituting the threonine residue at position 307 with an alanine residue (T307A).

In one embodiment, the Fc region is modified by substituting the threonine residue at position 307 with a proline residue (T307P).

In one embodiment, the Fc region is modified by substituting the valine residue at position 308 with a cysteine residue (V308C).

In one embodiment, the Fc region is modified by substituting the valine residue at position 308 with a phenylalanine residue (V308F).

In one embodiment, the Fc region is modified by substituting the valine residue at position 308 with a proline residue (V308P).

In one embodiment, the Fc region is modified by substituting the glutamine residue at position 311 with an alanine residue (Q311A).

In one embodiment, the Fc region is modified by substituting the glutamine residue at position 311 with an arginine residue (Q311R).

In one embodiment, the Fc region is modified by substituting the methionine residue at position 428 with a leucine residue (M428L).

In one embodiment, the Fc region is modified by substituting the histidine residue at position 433 with a lysine residue (H433K).

In one embodiment, the Fc region is modified by substituting the asparagine residue at position 434 with a phenylalanine residue (N434F).

In one embodiment, the Fc region is modified by substituting the asparagine residue at position 434 with a tyrosine residue (N434Y).

In one embodiment, the Fc region is modified by substituting the methionine residue at position 252 with a tyrosine residue, the serine residue at position 254 with a threonine residue, and the threonine residue at position 256 with a glutamic acid residue (M252Y/S254T/T256E).

In one embodiment, the Fc region is modified by substituting the valine residue at position 308 with a proline residue and the asparagine residue at position 434 with a tyrosine residue (V308P/N434Y).

In one embodiment, the Fc region is modified by substituting the methionine residue at position 252 with a tyrosine residue, the serine residue at position 254 with a threonine residue, the threonine residue at position 256 with a glutamic acid residue, the histidine residue at position 433 with a lysine residue and the asparagine residue at position 434 with a phenylalanine residue (M252Y/S254T/T256E/H433K/N434F).

It will be appreciated that any of the modifications listed above may be combined to alter FcRn binding.

In one embodiment, the MBM comprises an Fc domain in which one or both Fc regions comprise one or more modifications such that the Fc domain binds to FcRn with lower affinity and avidity than the corresponding native immunoglobulin.

In one embodiment, the Fc region comprises any amino acid residue other than histidine at position 310 and/or position 435.

The MBM of the disclosure may comprise an Fc domain in which one or both Fc regions comprise one or more modifications which increase its binding to FcγRIIb. FcγRIIb is the only inhibitory receptor in humans and the only Fc receptor found on B cells.

In one embodiment, the Fc region is modified by substituting the proline residue at position 238 with an aspartic acid residue (P238D).

In one embodiment, the Fc region is modified by substituting the glutamic acid residue at position 258 with an alanine residue (E258A).

In one embodiment, the Fc region is modified by substituting the serine residue at position 267 with an alanine residue (S267A).

In one embodiment, the Fc region is modified by substituting the serine residue at position 267 with a glutamic acid residue (S267E).

In one embodiment, the Fc region is modified by substituting the leucine residue at position 328 with a phenylalanine residue (L328F).

In one embodiment, the Fc region is modified by substituting the glutamic acid residue at position 258 with an alanine residue and the serine residue at position 267 with an alanine residue (E258A/S267A).

In one embodiment, the Fc region is modified by substituting the serine residue at position 267 with a glutamic acid residue and the leucine residue at position 328 with a phenylalanine residue (S267E/L328F).

It will be appreciated that any of the modifications listed above may be combined to increase FcγRIIb binding.

In one embodiment, MBMs are provided comprising Fc domains which display decreased binding to FcγR.

In one embodiment, an MBM comprises an Fc domain in which one or both Fc regions comprise one or more modifications that decrease Fc binding to FcγR.

The Fc domain can be derived from IgG1.

In one embodiment, the Fc region is modified by substituting the leucine residue at position 234 with an alanine residue (L234A).

In one embodiment, the Fc region is modified by substituting the leucine residue at position 235 with an alanine residue (L235A).

In one embodiment, the Fc region is modified by substituting the glycine residue at position 236 with an arginine residue (G236R).

In one embodiment, the Fc region is modified by substituting the asparagine residue at position 297 with an alanine residue (N297A) or a glutamine residue (N297Q).

In one embodiment, the Fc region is modified by substituting the serine residue at position 298 with an alanine residue (S298A).

In one embodiment, the Fc region is modified by substituting the leucine residue at position 328 with an arginine residue (L328R).

In one embodiment, the Fc region is modified by substituting the leucine residue at position 234 with an alanine residue and the leucine residue at position 235 with an alanine residue (L234A/L235A).

In one embodiment, the Fc region is modified by substituting the phenylalanine residue at position 234 with an alanine residue and the leucine residue at position 235 with an alanine residue (F234A/L235A).

In one embodiment, the Fc region is modified by substituting the glycine residue at position 236 with an arginine residue and the leucine residue at position 328 with an arginine residue (G236R/L328R).

It will be appreciated that any of the modifications listed above may be combined to decrease FcγR binding.

In one embodiment, a MBM comprises an Fc domain in which one or both Fc regions comprise one or more modifications that decrease Fc binding to FcγRIIIa without affecting the Fc's binding to FcγRII.

In one embodiment, the Fc region is modified by substituting the serine residue at position 239 with an alanine residue (S239A).

In one embodiment, the Fc region is modified by substituting the glutamic acid residue at position 269 with an alanine residue (E269A).

In one embodiment, the Fc region is modified by substituting the glutamic acid residue at position 293 with an alanine residue (E293A).

In one embodiment, the Fc region is modified by substituting the tyrosine residue at position 296 with a phenylalanine residue (Y296F).

In one embodiment, the Fc region is modified by substituting the valine residue at position 303 with an alanine residue (V303A).

In one embodiment, the Fc region is modified by substituting the alanine residue at position 327 with a glycine residue (A327G).

In one embodiment, the Fc region is modified by substituting the lysine residue at position 338 with an alanine residue (K338A).

In one embodiment, the Fc region is modified by substituting the aspartic acid residue at position 376 with an alanine residue (D376A).

It will be appreciated that any of the modifications listed above may be combined to decrease FcγRIIIa binding.

### 7.3.1.2. Fc Domains with Altered Complement Binding

An MBM (e.g., TBM) of the disclosure may comprise an Fc domain in which one or both Fc regions comprises one or more modifications that alter Fc binding to complement. Altered complement binding may be increased binding or decreased binding.

In one embodiment the Fc region comprises one or more modifications which decrease its binding to Clq. Initiation of the classical complement pathway starts with binding of hexameric Clq protein to the CH2 domain of antigen bound IgG and IgM.

In one embodiment, the MBM of the disclosure comprises an Fc domain in which one or both Fc regions comprises one or more modifications to decrease Fc binding to Clq.

In one embodiment, the Fc region is modified by substituting the leucine residue at position 234 with an alanine residue (L234A).

In one embodiment, the Fc region is modified by substituting the leucine residue at position 235 with an alanine residue (L235A).

In one embodiment, the Fc region is modified by substituting the leucine residue at position 235 with a glutamic acid residue (L235E).

In one embodiment, the Fc region is modified by substituting the glycine residue at position 237 with an alanine residue (G237A).

In one embodiment, the Fc region is modified by substituting the lysine residue at position 322 with an alanine residue (K322A).

In one embodiment, the Fc region is modified by substituting the proline residue at position 331 with an alanine residue (P331A).

In one embodiment, the Fc region is modified by substituting the proline residue at position 331 with a serine residue (P331S).

In one embodiment, a MBM of the disclosure comprises an Fc domain derived from IgG4. IgG4 has a naturally lower complement activation profile than IgG1, but also weaker binding of FcγR. Thus, in one embodiment, the MBM comprises an IgG4 Fc domain and also comprises one or more modifications that increase FcγR binding.

It will be appreciated that any of the modifications listed above may be combined to reduce Clq binding.

### 7.3.1.3. Fc Domains with Altered Disulfide Architecture

An MBM (e.g., TBM) of the disclosure can include an Fc domain comprising one or more modifications to create and/or remove a cysteine residue. Cysteine residues have an important role in the spontaneous assembly of Fc-based multispecific binding molecules, by forming disulfide bridges between individual pairs of polypeptide monomers. Thus, by altering the number and/or position of cysteine residues, it is possible to modify the structure of the MBM to produce a protein with improved therapeutic properties.

A MBM of the present disclosure can comprise an Fc domain in which one or both Fc regions, e.g., both Fc regions, comprise a cysteine residue at position 309. In one embodiment, the cysteine residue at position 309 is created by a modification, e.g., for an Fc domain derived from IgG1, the leucine residue at position 309 is substituted with a cysteine residue (L309C), for an Fc domain derived from IgG2, the valine residue at position 309 is substituted with a cysteine residue (V309C).

In one embodiment, the Fc region is modified by substituting the valine residue at position 308 with a cysteine residue (V308C).

In one embodiment, two disulfide bonds in the hinge region are removed by mutating a core hinge sequence CPPC to SPPS.

### 7.3.1.4. Fc Domains with Altered Glycosylation

In certain aspects, MBMs (e.g., TBMs) with improved manufacturability are provided that comprise fewer glycosylation sites than a corresponding immunoglobulin. These proteins have less complex post translational glycosylation patterns and are thus simpler and less expensive to manufacture.

In one embodiment, a glycosylation site in the CH2 domain is removed by substituting the asparagine residue at position 297 with an alanine residue (N297A) or a glutamine residue (N297Q). In addition to improved manufacturability, these aglycosyl mutants also reduce FcγR binding as described herein above.

In some embodiments, a MBM can be made that has an altered type of glycosylation, such as a hypofucosylated antibody having reduced amounts of fucosyl residues or an antibody having increased bisecting GlcNac structures. Such altered glycosylation patterns have been demonstrated to increase the ADCC ability of antibodies. Such carbohydrate modifications can be accomplished by, for example, expressing a MBM in a host cell with altered glycosylation machinery. Cells with altered glycosylation machinery have been described in the art and can be used as host cells in which to express MBMs of the disclosure to thereby produce MBM with altered glycosylation. For example, EP 1,176,195 by Hang et al. describes a cell line with a functionally disrupted FUT8 gene, which encodes a fucosyl transferase, such that antibodies expressed in such a cell line exhibit hypofucosylation. PCT Publication WO 03/035835 by Presta describes a variant CHO cell line, Lec13 cells, with reduced ability to attach fucose to Asn(297)-linked carbohydrates, also resulting in hypofucosylation of antibodies expressed in that host cell (see also Shields et al., 2002, J. Biol. Chem. 277:26733-26740). PCT Publication WO 99/54342 by Umana et al. describes cell lines engineered to express glycoprotein-modifying glycosyl transferases (e.g., beta(1,4)-N acetylglucosaminyltransferase III (GnTIII)) such that antibodies expressed in the engineered cell lines exhibit increased bisecting GlcNac structures which results in increased ADCC activity of the antibodies (see also Umana et al., Nat. Biotech. 17:176-180, 1999).

### 7.3.1.5. Fc Heterodimerization

Many multispecific molecule formats entail dimerization between two Fc regions that, unlike a native immunoglobulin, are operably linked to non-identical antigen-binding domains (or portions thereof, e.g., a VH or VH—CH1 of a Fab). Inadequate heterodimerization of two Fc regions to form an Fc domain has always been an obstacle for increasing the yield of desired multispecific molecules and represents challenges for purification. A variety of approaches available in the art can be used in for enhancing dimerization of Fc regions that might be present in the MBMs (e.g., TBMs) of the disclosure, for example as disclosed in EP 1870459A1; U.S. Pat. Nos. 5,582,996; 5,731,168; 5,910,573; 5,932,448; 6,833,441; 7,183,076; U.S. Patent Application Publication No. 2006204493A1; and PCT Publication No. WO2009/089004A1.

The present disclosure provides MBMs (e.g., TBMs) comprising Fc heterodimers, i.e., Fc domains comprising heterologous, non-identical Fc regions. Heterodimerization strategies are used to enhance dimerization of Fc regions operably linked to different ABMs (or portions thereof, e.g., a VH or VH—CH1 of a Fab) and reduce dimerization of Fc regions operably linked to the same ABM or portion thereof. Typically, each Fc region in the Fc heterodimer comprises a CH3 domain of an antibody. The CH3 domains are derived from the constant region of an antibody of any isotype, class or subclass, and in some cases, of IgG (IgG1, IgG2, IgG3 and IgG4) class, as described in the preceding section.

Typically, the MBMs comprise other antibody fragments in addition to CH3 domains, such as, CH1 domains, CH2 domains, hinge domain, VH domain(s), VL domain(s), CDR(s), and/or antigen-binding fragments described herein. In some embodiments, the two hetero-polypeptides are two heavy chains forming a bispecific or multispecific molecules. Heterodimerization of the two different heavy chains at CH3 domains give rise to the desired antibody or antibody-like molecule, while homodimerization of identical heavy chains will reduce yield of the desired antibody or molecule. In an exemplary embodiment, the two or more hetero-polypeptide chains comprise two chains comprising CH3 domains and forming the molecules of any of the multispecific molecule formats described above of the present disclosure. In an embodiment, the two hetero-polypeptide chains comprising CH3 domains comprise modifications that favor heterodimeric association of the polypeptides, relative to unmodified chains. Various examples of modification strategies are provided below in Table 2 and Sections 7.3.1.5.1 to 7.3.1.5.3.

TABLE 2
Fc Heterodimerization Strategies

| NO. | STRATEGY | CH3 DOMAIN 1 | CH3 DOMAIN 2 | REFERENCES |
|---|---|---|---|---|
| Fc 1 | knobs-into-holes | T366Y | Y407T | Ridgway et al., 1996, |
| | (Y-T) | | | Protein Eng 9: 617-21 |
| Fc 2 | knobs-into-holes | S354C, T366W | Y349C, T366S, | Atwell et al., 1997, |
| | (CW-CSAV) | | L368A, Y407V | J Mol Biol. 270(1): 26-35; |
| | | | | Merchant et al., 1998, |
| | | | | Nat Biotechnol 16: 677-681 |
| Fc 3 | HA-TF | S364H, F405A | Y349T, T394F | Moore et al., 2011, |
| | | | | MAbs 3(6): 546-57 |
| Fc 4 | ZW1 | T350V, L351Y, | T350V, T366L, | Von Kreudenstein et al., |
| | (VYAV-VLLW) | F405A, Y407V | K392L, T394W | 2013, MAbs 5: 646-54 |
| Fc 5 | CH3 charge pairs | K392D, K409D | E356K, D399K | Gunasekaran et al., 2010, |
| | (DD-KK) | | | J Biol Chem 285: 19637-46 |
| Fc 6 | IgG1 hingE, CH3 | IgG1: D221E, | IgG1: D221R, | Strop et al., 2012, |
| | charge pairs | P228E, L368E | P228R, K409R | J Mol Biol 420: 204-19 |
| | (EEE-RRR) | | | |
| Fc 7 | IgG2 hingE, CH3 | IgG2: C223E, | IgG2: C223R, | Strop et al., 2012, |
| | charge pairs | P228E, L368E | E225R, P228R, | J Mol Biol 420: 204-19 |
| | (EEE-RRRR) | | K409R | |
| Fc 8 | EW-RVT | K360E, K409W, | Q347R, D399V, | Choi et al., 2013, Mol |
| | | | F405T | Cancer Ther 12: 2748-59 |
| Fc 9 | EW-RVTS-S | K360E, K409W, | Q347R, D399V, | Choi et al., 2015, Mol |
| | | Y349C | F405T, S354C | Immunol 65: 377-83 |
| Fc 10 | Biclonic | 366K | 351D or E or D at | Geuijen et al., 2014, |
| | | (+351K) | 349, 368, 349, or | Journal of Clinical |
| | | | 349 + 355 | Oncology 32: suppl: 560 |
| Fc 11 | DuoBody | F405L | K409R | Labrijn et al., 2013, |
| | (L-R) | | | Proc Natl Acad Sci |
| | | | | USA 110: 5145-50 |
| Fc 12 | SEEDbody | IgG/A chimera | IgG/A chimera | Davis et al., 2010, |
| | | | | Protein Eng Des Sel |
| | | | | 23: 195-202 |
| Fc 13 | BEAT | residues from | residues from | Moretti et al., 2013, |
| | | TCRα interface | TCRβ interface | BMC Proceedings |
| | | | | 7(Suppl 6): O9 |
| Fc 14 | 7.8.60 | K360D, D399M, | E345R, Q347R, | Leaver-Fey et al., |
| | (DMA-RRVV) | Y407A | T366V, K409V | Structure 24: 641-51 |
| Fc 15 | 20.8.34 | Y349S, K370Y, | E356G, E357D, | Leaver-Fey et al., |
| | (SYMV-GDQA) | T366M, K409V | S364Q, Y407A | Structure 24: 641-51 |
| Fc 16 | Skew variant | None | none | FIG. 34 of US |
| | 12757 | | | 2016/0355600 |
| Fc 17 | Skew variant | L368D, K370S | S364K | FIG. 34 of US |
| | 12758 | | | 2016/0355600 |
| Fc 18 | Skew variant | L368D, K370S | S364K, E357L | FIG. 34 of US |
| | 12759 | | | 2016/0355600 |
| Fc 19 | Skew variant | L368D, K370S | S364K, E357Q | FIG. 34 of US |
| | 12760 | | | 2016/0355600 |
| Fc 20 | Skew variant | T411E, K360E, | D401K | FIG. 34 of US |
| | 12761 | Q362E | | 2016/0355600 |
| Fc 21 | Skew variant | L368E, K370S | S364K | FIG. 34 of US |
| | 12496 | | | 2016/0355600 |
| Fc 22 | Skew variant | K370S | S364K | FIG. 34 of US |
| | 12511 | | | 2016/0355600 |
| Fc 23 | Skew variant | L368E, K370S | S364K, E357Q | FIG. 34 of US |
| | 12840 | | | 2016/0355600 |
| Fc 24 | Skew variant | K370S | S364K, E357Q | FIG. 34 of US |
| | 12841 | | | 2016/0355600 |
| Fc 25 | Skew variant | L368E, K370S | S364K | FIG. 34 of US |
| | 12894 | | | 2016/0355600 |
| Fc 26 | Skew variant | K370S | S364K | FIG. 34 of US |
| | 12895 | | | 2016/0355600 |
| Fc 27 | Skew variant | L368E, K370S | S364K, E357Q | FIG. 34 of US |
| | 12896 | | | 2016/0355600 |
| Fc 28 | Skew variant | K370S | S364K, E357Q | FIG. 34 of US |
| | 12901 | | | 2016/0355600 |
| Fc 29 | pl_ISO(−) | I199T, N203D, | | FIG. 31 of US |
| | | K274Q, R355Q, | | 2016/0355600 |
| | | N384S, K392N, | | |
| | | V397M, Q419E, | | |
| | | DEL447 | | |
| Fc 30 | pl_(−)_Isosteric_A | N208D, Q295E, | | FIG. 31 of US |
| | | N384D, Q418E, | | 2016/0355600 |
| | | N421D | | |
| Fc 31 | pl_(−)_isosteric_B | N208D, Q295E, | | FIG. 31 of US |
| | | Q418E, N421D | | 2016/0355600 |
| Fc 32 | pl_ISO(+RR) | Q196K, I199T, | | FIG. 31 of US |
| | | P217R, P228R, | | 2016/0355600 |
| | | N276K | | |
| Fc 33 | pl_ISO(+) | Q196K, I199T, | | FIG. 31 of US |
| | | N276K | | 2016/0355600 |
| Fc 34 | pl_(+) isosteric_A | E269Q, E272Q, | | FIG. 31 of US |
| | | E283Q, E357Q, | | 2016/0355600 |
| Fc 35 | pl_(+)_isosteric_B | E269Q, E272Q, | | FIG. 31 of US |
| | | E283Q | | 2016/0355600 |
| Fc 36 | pl_ (+) | E269Q, E272Q | | FIG. 31 of US |
| | isosteric_E269Q, | | | 2016/0355600 |
| | E272Q | | | |
| Fc 37 | pl_(+)_isosteric_E | E269Q, E283Q | | FIG. 31 of US |
| | 269Q, E283Q | | | 2016/0355600 |
| Fc 38 | pl_(+) | E272Q, E283Q | | FIG. 31 of US |
| | isosteric_E2720, | | | 2016/0355600 |
| | E283Q | | | |
| Fc 39 | pl_(+)_isosteric_E | E269Q | | FIG. 31 of US |
| | 269Q | | | 2016/0355600 |
| Fc 40 | Heterodimerization | F405A | T394F | FIG. 30A of US |
| | | | | 2016/0355600 |
| Fc 41 | Heterodimerization | S364D | Y349K | FIG. 30A of US |
| | | | | 2016/0355600 |
| Fc 42 | Heterodimerization | S364E | L368K | FIG. 30A of US |
| | | | | 2016/0355600 |
| Fc 43 | Heterodimerization | S364E | Y349K | FIG. 30A of US |
| | | | | 2016/0355600 |
| Fc 44 | Heterodimerization | S364F | K370G | FIG. 30A of US |
| | | | | 2016/0355600 |
| Fc 45 | Heterodimerization | S364H | Y349K | FIG. 30A of US |
| | | | | 2016/0355600 |
| Fc 46 | Heterodimerization | S364H | Y349T | FIG. 30A of US |
| | | | | 2016/0355600 |
| Fc 47 | Heterodimerization | S364Y | K370G | FIG. 30A of US |
| | | | | 2016/0355600 |
| Fc 48 | Heterodimerization | T411K | K370E | FIG. 30A of US |
| | | | | 2016/0355600 |
| Fc 49 | Heterodimerization | V397S, F405A | T394F | FIG. 30A of US |
| | | | | 2016/0355600 |
| Fc 50 | Heterodimerization | K370R, T411K | K370E, T411E | FIG. 30A of US |
| | | | | 2016/0355600 |
| Fc 51 | Heterodimerization | L351E, S364D | Y349K, L351K | FIG. 30A of US |
| | | | | 2016/0355600 |
| Fc 52 | Heterodimerization | L351E, S364E | Y349K, L351K | FIG. 30A of US |
| | | | | 2016/0355600 |
| Fc 53 | Heterodimerization | L351E, T366D | L351K, T366K | FIG. 30A of US |
| | | | | 2016/0355600 |
| Fc 54 | Heterodimerization | P395T, V397S, | T394F | FIG. 30A of US |
| | | F405A | | 2016/0355600 |
| Fc 55 | Heterodimerization | S364D, K370G | S364Y, K370R | FIG. 30A of US |
| | | | | 2016/0355600 |
| Fc 56 | Heterodimerization | S364D, T394F | Y349K, F405A | FIG. 30A of US |
| | | | | 2016/0355600 |
| Fc 57 | Heterodimerization | S364E, F405A | Y349K, T394F | FIG. 30A of US |
| | | | | 2016/0355600 |
| Fc 58 | Heterodimerization | S364E, F405S | Y349K, T394Y | FIG. 30A of US |
| | | | | 2016/0355600 |
| Fc 59 | Heterodimerization | S364E, T411E | Y349K, D401K | FIG. 30A of US |
| | | | | 2016/0355600 |
| Fc 60 | Heterodimerization | S364H, D401K | Y349T, T411E | FIG. 30A of US |
| | | | | 2016/0355600 |
| Fc 61 | Heterodimerization | S364H, F405A | Y349T, T394F | FIG. 30A of US |
| | | | | 2016/0355600 |
| Fc 62 | Heterodimerization | S364H, T394F | Y349T, F405A | FIG. 30A of US |
| | | | | 2016/0355600 |
| Fc 63 | Heterodimerization | Y349C, S364E | Y349K, S354C | FIG. 30A of US |
| | | | | 2016/0355600 |
| Fc 64 | Heterodimerization | L351E, S364D, | Y349K, L351K, | FIG. 30A of US |
| | | F405A | T394F | 2016/0355600 |
| Fc 65 | Heterodimerization | L351K, S364H, | Y349T, L351E, | FIG. 30A of US |
| | | D401K | T411E | 2016/0355600 |
| Fc 66 | Heterodimerization | S364E, T411E, | Y349K, T394F, | FIG. 30A of US |
| | | F405A | D401K | 2016/0355600 |
| Fc 67 | Heterodimerization | S364H, D401K, | Y349T, T394F, | FIG. 30A of US |
| | | F405A | T411E | 2016/0355600 |
| Fc 68 | Heterodimerization | S364H, F405A, | Y349T, T394F, | FIG. 30A of US |
| | | T411E | D401K | 2016/0355600 |
| Fc 69 | Heterodimerization | T411E, K360E, | D401K | FIG. 30C of US |
| | | N390D | | 2016/0355600 |
| Fc 70 | Heterodimerization | T411E, Q362E, | D401K | FIG. 30C of US |
| | | N390D | | 2016/0355600 |
| Fc 71 | Heterodimerization | T411E, Q347R | D401K, K360D | FIG. 30C of US |
| | | | | 2016/0355600 |
| Fc 72 | Heterodimerization | T411E, Q347R | D401K, K360E | FIG. 30C of US |
| | | | | 2016/0355600 |
| Fc 73 | Heterodimerization | T411E, K360 | D401K, Q347K | FIG. 30C of US |
| | | | | 2016/0355600 |
| Fc 74 | Heterodimerization | T411E, K360D | D401K, Q347R | FIG. 30C of US |
| | | | | 2016/0355600 |
| Fc 75 | Heterodimerization | T411E, K360E | D401K, Q347K | FIG. 30C of US |
| | | | | 2016/0355600 |
| Fc 76 | Heterodimerization | T411E, K360E | D401K, Q347R | FIG. 30C of US |
| | | | | 2016/0355600 |
| Fc 77 | Heterodimerization | T411E, S364K | D401K, K370S | FIG. 30C of US |
| | | | | 2016/0355600 |
| Fc 78 | Heterodimerization | T411E, K370S | D401K, S364K | FIG. 30C of US |
| | | | | 2016/0355600 |
| Fc 79 | Heterodimerization | Q347E | E357Q | FIG. 30C of US |
| | | | | 2016/0355600 |
| Fc 80 | Heterodimerization | Q347E | E357Q, Q362K | FIG. 30C of US |
| | | | | 2016/0355600 |
| Fc 81 | Heterodimerization | K360D, Q362E | Q347R | FIG. 30C of US |
| | | | | 2016/0355600 |
| Fc 82 | Heterodimerization | K360D, Q362E | D401K | FIG. 30C of US |
| | | | | 2016/0355600 |
| Fc 83 | Heterodimerization | K360D, Q362E | Q347R, D401K | FIG. 30C of US |
| | | | | 2016/0355600 |
| Fc 84 | Heterodimerization | K360E, Q362E | Q347R | FIG. 30C of US |
| | | | | 2016/0355600 |
| Fc 85 | Heterodimerization | K360E, Q362E | D401K | FIG. 30C of US |
| | | | | 2016/0355600 |
| Fc 86 | Heterodimerization | K360E, Q362E | Q347R, D401K | FIG. 30C of US |
| | | | | 2016/0355600 |
| Fc 87 | Heterodimerization | Q362E, N390D | D401K | FIG. 30C of US |
| | | | | 2016/0355600 |
| Fc 88 | Heterodimerization | Q347E, K360D | D401N | FIG. 30C of US |
| | | | | 2016/0355600 |
| Fc 89 | Heterodimerization | K360D | Q347R, N390K | FIG. 30C of US |
| | | | | 2016/0355600 |
| Fc 90 | Heterodimerization | K360D | N390K, D401N | FIG. 30C of US |
| | | | | 2016/0355600 |
| Fc 91 | Heterodimerization | K360E | Y349H | FIG. 30C of US |
| | | | | 2016/0355600 |
| Fc 92 | Heterodimerization | K370S, Q347E | S364K | FIG. 30C of US |
| | | | | 2016/0355600 |
| Fc 93 | Heterodimerization | K370S, E357L | S364K | FIG. 30C of US |
| | | | | 2016/0355600 |
| Fc 94 | Heterodimerization | K370S, E357Q | S364K | FIG. 30C of US |
| | | | | 2016/0355600 |
| Fc 95 | Heterodimerization | K370S, Q347E, | S364K | FIG. 30C of US |
| | | E357L | | 2016/0355600 |
| Fc 96 | Heterodimerization | K370S, Q347E, | S364K | FIG. 30C of US |
| | | E357Q | | 2016/0355600 |
| Fc 97 | Heterodimerization | L368D, K370S, | S364K | FIG. 30D of US |
| | | Q347E | | 2016/0355600 |
| Fc 98 | Heterodimerization | L368D, K370S, | S364K | FIG. 30D of US |
| | | E357L | | 2016/0355600 |
| Fc 99 | Heterodimerization | L368D, K370S, | S364K | FIG. 30D of US |
| | | E357Q | | 2016/0355600 |
| Fc 100 | Heterodimerization | L368D, K370S, | S364K | FIG. 30D of US |
| | | Q347E, E357L | | 2016/0355600 |
| Fc 101 | Heterodimerization | L368D, K370S, | S364K | FIG. 30D of US |
| | | Q347E, E357Q | | 2016/0355600 |
| Fc 102 | Heterodimerization | L368E, K370S, | S364K | FIG. 30D of US |
| | | Q347E | | 2016/0355600 |
| Fc 103 | Heterodimerization | L368E, K370S, | S364K | FIG. 30D of US |
| | | E357L | | 2016/0355600 |
| Fc 104 | Heterodimerization | L368E, K370S, | S364K | FIG. 30D of US |
| | | E357Q | | 2016/0355600 |
| Fc 105 | Heterodimerization | L368E, K370S, | S364K | FIG. 30D of US |
| | | Q347E, E357L | | 2016/0355600 |
| Fc 106 | Heterodimerization | L368E, K370S, | S364K | FIG. 30D of US |
| | | Q347E, E357Q | | 2016/0355600 |
| Fc 107 | Heterodimerization | L368D, K370T, | S364K | FIG. 30D of US |
| | | Q347E | | 2016/0355600 |
| Fc 108 | Heterodimerization | L368D, K370T, | S364K | FIG. 30D of US |
| | | E357L | | 2016/0355600 |
| Fc 109 | Heterodimerization | L368D, K370T, | S364K | FIG. 30D of US |
| | | E357Q | | 2016/0355600 |
| Fc 110 | Heterodimerization | L368D, K370T, | S364K | FIG. 30D of US |
| | | Q347E, E357L | | 2016/0355600 |
| Fc 111 | Heterodimerization | L368D, K370T, | S364K | FIG. 30D of US |
| | | Q347E, E357Q | | 2016/0355600 |
| Fc 112 | Heterodimerization | L368E, K370T, | S364K | FIG. 30D of US |
| | | Q347E | | 2016/0355600 |
| Fc 113 | Heterodimerization | L368E, K370T, | S364K | FIG. 30D of US |
| | | E357L | | 2016/0355600 |
| Fc 114 | Heterodimerization | L368E, K370T, | S364K | FIG. 30D of US |
| | | E357Q | | 2016/0355600 |
| Fc 115 | Heterodimerization | L368E, K370T, | S364K | FIG. 30D of US |
| | | Q347E, E357L | | 2016/0355600 |
| Fc 116 | Heterodimerization | L368E, K370T, | S364K | FIG. 30D of US |
| | | Q347E, E357Q | | 2016/0355600 |
| Fc 117 | Heterodimerization | T411E, Q362E | D401K, T411K | FIG. 30D of US |
| | | | | 2016/0355600 |
| Fc 118 | Heterodimerization | T411E, N390D | D401K, T411K | FIG. 30D of US |
| | | | | 2016/0355600 |
| Fc 119 | Heterodimerization | T411E, Q362E | D401R, T411R | FIG. 30D of US |
| | | | | 2016/0355600 |
| Fc 120 | Heterodimerization | T411E, N390D | D401R, T411R | FIG. 30D of US |
| | | | | 2016/0355600 |
| Fc 121 | Heterodimerization | Y407T | T366Y | FIG. 30D of US |
| | | | | 2016/0355600 |
| Fc 122 | Heterodimerization | F405A | T394W | FIG. 30D of US |
| | | | | 2016/0355600 |
| Fc 123 | Heterodimerization | T366Y, F405A | T394W, Y407T | FIG. 30D of US |
| | | | | 2016/0355600 |
| Fc 124 | Heterodimerization | T3665, L368A, | T366W | FIG. 30D of US |
| | | Y407V | | 2016/0355600 |
| Fc 125 | Heterodimerization | T366S, L368A, | T366W, S354C | FIG. 30D of US |
| | | Y407V, Y349C | | 2016/0355600 |
| Fc 126 | Heterodimerization | K392D, K409D | E356K, D399K | FIG. 30E of US |
| | | | | 2016/0355600 |
| Fc 127 | Heterodimerization | K370D, K392D, | E356K, E357K, | FIG. 30E of US |
| | | K409D | D399K | 2016/0355600 |
| Fc 128 | Heterodimerization | I199T, N203D, | Q196K, L99T, | FIG. 30E of US |
| | | K247Q, R355Q, | P217R, P228R, | 2016/0355600 |
| | | N384S, K392N, | N276K | |
| | | V397M, Q419E, | | |
| | | K447 | | |
| Fc 129 | Heterodimerization | I199T, N203D, | Q196K, L99T, | FIG. 30E of US |
| | | K247Q, R355Q, | N276K | 2016/0355600 |
| | | N384S, K392N, | | |
| | | V397M, Q419E, | | |
| | | K447 | | |
| Fc 130 | Heterodimerization | N384S, K392N, | N276K | FIG. 30E of US |
| | | V397M, Q419E | | 2016/0355600 |
| Fc 131 | Heterodimerization | D221E, P228E, | D221R, P228R, | FIG. 30E of US |
| | | L368E | K409R | 2016/0355600 |
| Fc 132 | Heterodimerization | C220E, P228E, | C220R, E224R, | FIG. 30E of US |
| | | L368E | P228R, K409R | 2016/0355600 |
| Fc 133 | Heterodimerization | F405L | K409R | FIG. 30E of US |
| | | | | 2016/0355600 |
| Fc 134 | Heterodimerization | T366I, K392M, | F405A, Y407V | FIG. 30E of US |
| | | T394W | | 2016/0355600 |
| Fc 135 | Heterodimerization | T366V, K409F | L351Y, Y407A | FIG. 30E of US |
| | | | | 2016/0355600 |
| Fc 136 | Heterodimerization | T366A, K392E, | D399R, S400R, | FIG. 30E of US |
| | | K409F, T411E | Y407A | 2016/0355600 |
| Fc 137 | Heterodimerization | L351K | L351E | FIG. 30E of US |
| | | | | 2016/0355600 |
| Fc 138 | Heterodimerization | I199T, N203D, | Q196K, L199T, | FIG. 30E of US |
| | | K247Q, R355Q, | P217R, P228R, | 2016/0355600 |
| | | Q419E, K447 | N276K | |
| Fc 139 | Heterodimerization | I199T, N203D, | Q196K, I199T, | FIG. 30E of US |
| | | K247Q, R355Q, | N276K | 2016/0355600 |
| | | Q419E, K447 | | |
| Fc 140 | Heterodimerization | I199T, N203D, | | FIG. 30E of US |
| | | K274Q, R355Q, | | 2016/0355600 |
| | | N384S, K392N, | | |
| | | V397M, Q419E | | |
| | | DEL447 | | |
| Fc 141 | Heterodimerization | N208D, Q295E | | FIG. 30E of US |
| | | N384D, Q418E | | 2016/0355600 |
| | | N421D | | |
| Fc 142 | Heterodimerization | N208D, Q295E | | FIG. 30E of US |
| | | Q418E, N421D | | 2016/0355600 |
| Fc 143 | Heterodimerization | Q196K, I199T | | FIG. 30E of US |
| | | P217R, P228R | | 2016/0355600 |
| | | N276K | | |
| Fc 144 | Heterodimerization | Q196K, I199T | | FIG. 30E of US |
| | | N276K | | 2016/0355600 |
| Fc 145 | Heterodimerization | E269Q, E272Q | | FIG. 30E of US |
| | | E283Q, E357Q | | 2016/0355600 |
| Fc 146 | Heterodimerization | E269Q, E272Q | | FIG. 30E of US |
| | | E283Q, | | 2016/0355600 |
| Fc 147 | Heterodimerization | E269Q, E272Q | | FIG. 30E of US |
| | | | | 2016/0355600 |
| Fc 148 | Heterodimerization | E269Q, E283Q | | FIG. 30E of US |
| | | | | 2016/0355600 |
| Fc 149 | Heterodimerization | E272Q, E283Q | | FIG. 30E of US |
| | | | | 2016/0355600 |
| Fc 150 | Heterodimerization | E269Q | | FIG. 30E of US |
| | | | | 2016/0355600 |

### 7.3.1.5.1. Knob-In-Hole (KIH)

MBMs (e.g., TBMs) of the disclosure may comprise one or more, e.g., a plurality, of modifications to one or more of the constant domains of an Fc domain, e.g., to the CH3 domains. In one example, a MBM (e.g., a TBM) of the present disclosure comprises two polypeptides that each comprise a heavy chain constant domain of an antibody, e.g., a CH2 or CH3 domain. In an example, the two heavy chain constant domains, e.g., the CH2 or CH3 domains of the MBM (e.g., TBM) comprise one or more modifications that allow for a heterodimeric association between the two chains. In one aspect, the one or more modifications are disposed on CH2 domains of the two heavy chains. In one aspect, the one or more modifications are disposed on CH3 domains of at least two polypeptides of the MBM. In one aspect, the one or more modifications to a first polypeptide of the MBM comprising a heavy chain constant domain can create a “knob” and the one or more modifications to a second polypeptide of the MBM creates a “hole,” such that heterodimerization of the polypeptide of the MBM comprising a heavy chain constant domain causes the “knob” to interface (e.g., interact, e.g., a CH2 domain of a first polypeptide interacting with a CH2 domain of a second polypeptide, or a CH3 domain of a first polypeptide interacting with a CH3 domain of a second polypeptide) with the “hole.” As the term is used herein, a “knob” refers to at least one amino acid side chain which projects from the interface of a first polypeptide of the MBM comprising a heavy chain constant domain and is therefore positionable in a compensatory “hole” in the interface with a second polypeptide of the MBM comprising a heavy chain constant domain so as to stabilize the heteromultimer, and thereby favor heteromultimer formation over homomultimer formation, for example. The knob may exist in the original interface or may be introduced synthetically (e.g. by altering nucleic acid encoding the interface). The import residues for the formation of a knob are generally naturally occurring amino acid residues and can be selected from arginine (R), phenylalanine (F), tyrosine (Y) and tryptophan (W). In some cases, tryptophan and tyrosine are selected. In an embodiment, the original residue for the formation of the protuberance has a small side chain volume, such as alanine, asparagine, aspartic acid, glycine, serine, threonine or valine.

A “hole” refers to at least one amino acid side chain which is recessed from the interface of a second polypeptide of the MBM comprising a heavy chain constant domain and therefore accommodates a corresponding knob on the adjacent interfacing surface of a first polypeptide of the MBM comprising a heavy chain constant domain. The hole may exist in the original interface or may be introduced synthetically (e.g. by altering nucleic acid encoding the interface). The import residues for the formation of a hole are usually naturally occurring amino acid residues and are preferably selected from alanine (A), serine (S), threonine (T) and valine (V). In one embodiment, the amino acid residue is serine, alanine or threonine. In another embodiment, the original residue for the formation of the hole has a large side chain volume, such as tyrosine, arginine, phenylalanine or tryptophan.

In an embodiment, a first CH3 domain is modified at residue 366, 405 or 407 to create either a “knob” or a hole” (as described above), and the second CH3 domain that heterodimerizes with the first CH3 domain is modified at: residue 407 if residue 366 is modified in the first CH3 domain, residue 394 if residue 405 is modified in the first CH3 domain, or residue 366 if residue 407 is modified in the first CH3 domain to create a “hole” or “knob” complementary to the “knob” or “hole” of the first CH3 domain.

In another embodiment, a first CH3 domain is modified at residue 366, and the second CH3 domain that heterodimerizes with the first CH3 domain is modified at residues 366, 368 and/or 407, to create a “hole” or “knob” complementary to the “knob” or “hole” of the first CH3 domain. In one embodiment, the modification to the first CH3 domain introduces a tyrosine (Y) residue at position 366. In an embodiment, the modification to the first CH3 is T366Y. In one embodiment, the modification to the first CH3 domain introduces a tryptophan (W) residue at position 366. In an embodiment, the modification to the first CH3 is T366W. In some embodiments, the modification to the second CH3 domain that heterodimerizes with the first CH3 domain modified at position 366 (e.g., has a tyrosine (Y) or tryptophan (W) introduced at position 366, e.g., comprises the modification T366Y or T366W), comprises a modification at position 366, a modification at position 368 and a modification at position 407. In some embodiments, the modification at position 366 introduces a serine (S) residue, the modification at position 368 introduces an alanine (A), and the modification at position 407 introduces a valine (V). In some embodiments, the modifications comprise T366S, L368A and Y407V. In one embodiment the first CH3 domain of the multispecific molecule comprises the modification T366Y, and the second CH3 domain that heterodimerizes with the first CH3 domain comprises the modifications T366S, L368A and Y407V, or vice versa. In one embodiment the first CH3 domain of the multispecific molecule comprises the modification T366W, and the second CH3 domain that heterodimerizes with the first CH3 domain comprises the modifications T366S, L368A and Y407V, or vice versa.

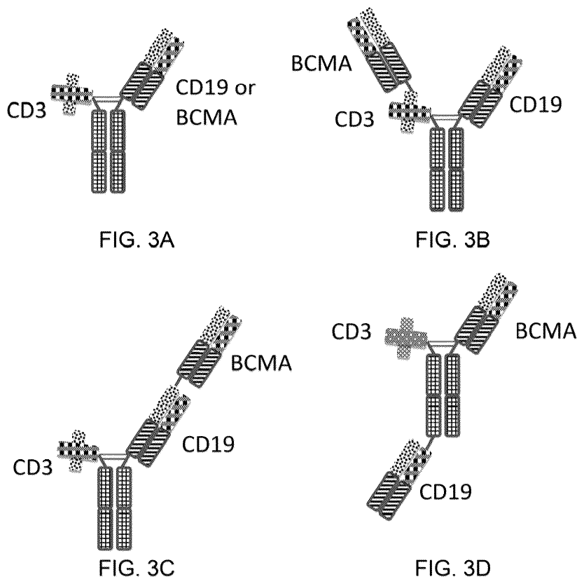

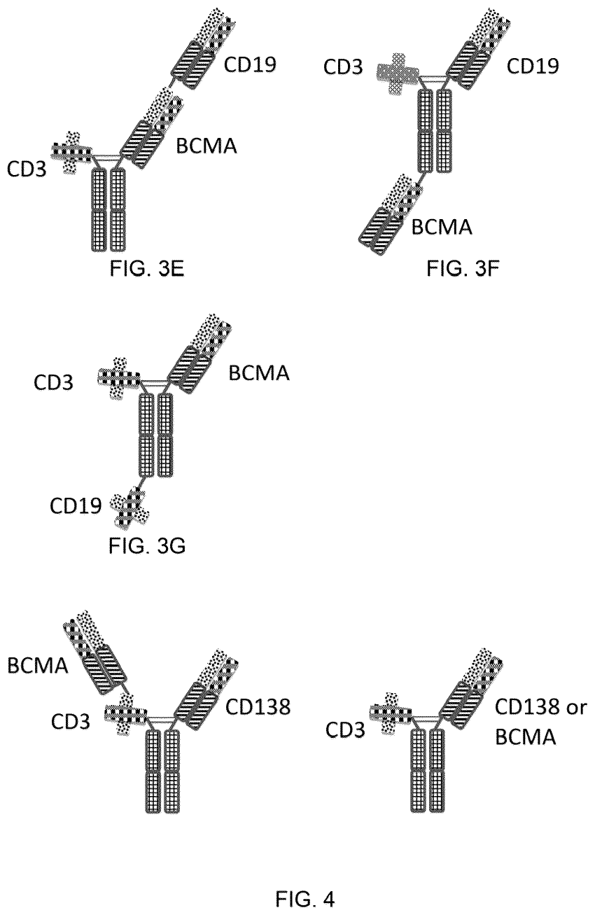

Additional steric or “skew” (e.g., knob in hole) modifications are described in PCT publication no. WO2014/145806 (for example, FIG. 3, FIG. 4 and FIG. 12 of WO2014/145806), PCT publication no. WO2014/110601, and PCT publication no. WO 2016/086186, WO 2016/086189, WO 2016/086196 and WO 2016/182751. An example of a KIH variant comprises a first constant chain comprising a L368D and a K370S modification, paired with a second constant chain comprising a S364K and E357Q modification.

Additional knob in hole modification pairs suitable for use in any of the multispecific molecules of the present disclosure are further described in, for example, WO1996/027011, and Merchant et al., 1998, Nat. Biotechnol., 16:677-681.

In further embodiments, the CH3 domains may be additionally modified to introduce a pair of cysteine residues. Without being bound by theory, it is believed that the introduction of a pair of cysteine residues capable of forming a disulfide bond provide stability to heterodimerized MBMs (e.g., TBMs) comprising paired CH3 domains. In some embodiments, the first CH3 domain comprises a cysteine at position 354, and the second CH3 domain that heterodimerizes with the first CH3 domain comprises a cysteine at position 349. In some embodiments, the first CH3 domain comprises a cysteine at position 354 (e.g., comprises the modification S354C) and a tyrosine (Y) at position 366 (e.g., comprises the modification T366Y), and the second CH3 domain that heterodimerizes with the first CH3 domain comprises a cysteine at position 349 (e.g., comprises the modification Y349C), a serine at position 366 (e.g., comprises the modification T366S), an alanine at position 368 (e.g., comprises the modification L368A), and a valine at position 407 (e.g., comprises the modification Y407V). In some embodiments, the first CH3 domain comprises a cysteine at position 354 (e.g., comprises the modification S354C) and a tryptophan (W) at position 366 (e.g., comprises the modification T366W), and the second CH3 domain that heterodimerizes with the first CH3 domain comprises a cysteine at position 349 (e.g., comprises the modification Y349C), a serine at position 366 (e.g., comprises the modification T366S), an alanine at position 368 (e.g., comprises the modification L368A), and a valine at position 407 (e.g., comprises the modification Y407V).

### 7.3.1.5.2. Alternative Knob and Hole: IgG Heterodimerization

Heterodimerization of polypeptide chains of a MBM (e.g., a TBM) comprising paired CH3 domains can be increased by introducing one or more modifications in a CH3 domain which is derived from the IgG1 antibody class. In an embodiment, the modifications comprise a K409R modification to one CH3 domain paired with F405L modification in the second CH3 domain. Additional modifications may also, or alternatively, be at positions 366, 368, 370, 399, 405, 407, and 409. In some cases, heterodimerization of polypeptides comprising such modifications is achieved under reducing conditions, e.g., 10-100 mM 2-MEA (e.g., 25, 50, or 100 mM 2-MEA) for 1-10, e.g., 1.5-5, e.g., 5, hours at 25-37 C, e.g., 25 C or 37 C.

The amino acid replacements described herein can be introduced into the CH3 domains using techniques which are well known in the art (see, e.g., McPherson, ed., 1991, Directed Mutagenesis: a Practical Approach; Adelman et al., 1983, DNA, 2:183).

The IgG heterodimerization strategy is further described in, for example, WO2008/119353, WO2011/131746, and WO2013/060867.

In any of the embodiments described in this Section, the CH3 domains can be additionally modified to introduce a pair of cysteine residues as described in Section 7.3.1.5.1.

### 7.3.1.5.3. Polar Bridge

Heterodimerization of polypeptide chains of MBMs (e.g., TBMs) comprising an Fc domain can be increased by introducing modifications based on the “polar-bridging” rationale, which is to make residues at the binding interface of the two polypeptide chains to interact with residues of similar (or complimentary) physical property in the heterodimer configuration, while with residues of different physical property in the homodimer configuration. In particular, these modifications are designed so that, in the heterodimer formation, polar residues interact with polar residues, while hydrophobic residues interact with hydrophobic residues. In contrast, in the homodimer formation, residues are modified so that polar residues interact with hydrophobic residues. The favorable interactions in the heterodimer configuration and the unfavorable interactions in the homodimer configuration work together to make it more likely for Fc regions to form heterodimers than to form homodimers.

In an exemplary embodiment, the above modifications are generated at one or more positions of residues 364, 368, 399, 405, 409, and 411 of a CH3 domain.

In some embodiments, one or more modifications selected from the group consisting of S364L, T366V, L368Q, N399K, F405S, K409F and R411K are introduced into one of the two CH3 domains. One or more modifications selected from the group consisting of Y407F, K409Q and T411N can be introduced into the second CH3 domain.

In another embodiment, one or more modifications selected from the group consisting of S364L, T366V, L368Q, D399K, F405S, K409F and T411K are introduced into one CH3 domain, while one or more modifications selected the group consisting of from Y407F, K409Q and T411D are introduced into the second CH3 domain.

In one exemplary embodiment, the original residue of threonine at position 366 of one CH3 domain is replaced by valine, while the original residue of tyrosine at position 407 of the other CH3 domain is replaced by phenylalanine.

In another exemplary embodiment, the original residue of serine at position 364 of one CH3 domain is replaced by leucine, while the original residue of leucine at position 368 of the same CH3 domain is replaced by glutamine.

In yet another exemplary embodiment, the original residue of phenylalanine at position 405 of one CH3 domain is replaced by serine and the original residue of lysine at position 409 of this CH3 domain is replaced by phenylalanine, while the original residue of lysine at position 409 of the other CH3 domain is replaced by glutamine.

In yet another exemplary embodiment, the original residue of aspartic acid at position 399 of one CH3 domain is replaced by lysine, and the original residue of threonine at position 411 of the same CH3 domain is replaced by lysine, while the original residue of threonine at position 411 of the other CH3 domain is replaced by aspartic acid.

The amino acid replacements described herein can be introduced into the CH3 domains using techniques which are well known in the art (see, e.g., McPherson, ed., 1991, Directed Mutagenesis: a Practical Approach; Adelman et al., 1983, DNA, 2:183). The polar bridge strategy is described in, for example, WO2006/106905, WO2009/089004 and K. Gunasekaran, et al. (2010) JBC, 285:19637-19646.

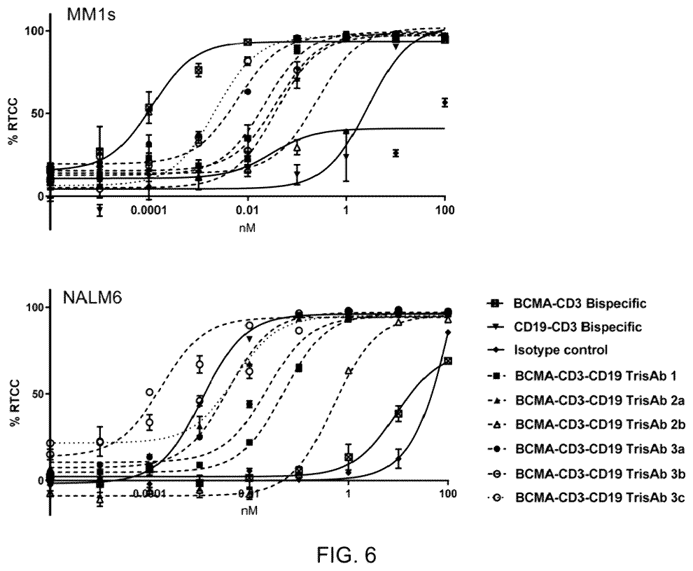

Additional polar bridge modifications are described in, for example, PCT publication no. WO2014/145806 (for example, FIG. 6 of WO2014/145806), PCT publication no. WO2014/110601, and PCT publication no. WO 2016/086186, WO 2016/086189, WO 2016/086196 and WO 2016/182751. An example of a polar bridge variant comprises a constant chain comprising a N208D, Q295E, N384D, Q418E and N421D modification.

In any of the embodiments described herein, the CH3 domains may be additionally modified to introduce a pair of cysteine residues as described in Section 7.3.1.5.1.

Additional strategies for enhancing heterodimerization are described in, for example, WO2016/105450, WO2016/086186, WO2016/086189, WO2016/086196, WO2016/141378, and WO2014/145806, and WO2014/110601. Any of the strategies can be employed in a MBM described herein.

### 7.3.2. Hinge Regions

The MBMs (e.g., TBMs) of the disclosure can also comprise hinge regions, e.g., connecting an antigen-binding module to an Fc region. The hinge region can be a native or a modified hinge region. Hinge regions are typically found at the N-termini of Fc regions.

A native hinge region is the hinge region that would normally be found between Fab and Fc domains in a naturally occurring antibody. A modified hinge region is any hinge that differs in length and/or composition from the native hinge region. Such hinges can include hinge regions from other species, such as human, mouse, rat, rabbit, shark, pig, hamster, camel, llama or goat hinge regions. Other modified hinge regions may comprise a complete hinge region derived from an antibody of a different class or subclass from that of the heavy chain Fc region. Alternatively, the modified hinge region may comprise part of a natural hinge or a repeating unit in which each unit in the repeat is derived from a natural hinge region. In a further alternative, the natural hinge region may be altered by converting one or more cysteine or other residues into neutral residues, such as serine or alanine, or by converting suitably placed residues into cysteine residues. By such means the number of cysteine residues in the hinge region may be increased or decreased. This approach is described further in U.S. Pat. No. 5,677,425 by Bodmer et al., Altering the number of cysteine residues in a hinge region can, for example, facilitate assembly of light and heavy chains, or increase or decrease the stability of a MBM. Other modified hinge regions may be entirely synthetic and may be designed to possess desired properties such as length, cysteine composition and flexibility.

A number of modified hinge regions have been described for example, in U.S. Pat. No. 5,677,425, WO9915549, WO2005003170, WO2005003169, WO2005003170, WO9825971 and WO2005003171.

Examples of suitable hinge sequences are shown in Table 3.

TABLE 3
Hinge Sequences

| Hinge | Hinge | | SEQ ID |
|---|---|---|---|
| Name | Description | Hinge Sequence | NO: |
| H1 | Human IgA1 | VPSTPPTPSPSTPPTPSPS | 1 |
| H2 | Human IgA2 | VPPPPP | 2 |
| H3 | Human IgD | ESPKAQASSVPTAQPQAEG | 3 |
| | | SLAKATTAPATTRNTGRGG | |
| | | EEKKKEKEKEEQEERETKT | |
| | | P | |
| H4 | Human IgG1 | EPKSCDKTHTCPPCP | 4 |
| H5 | Human IgG2 | ERKCCVECPPCP | 5 |
| H6 | Human IgG3 | ELKTPLGDTTHTCPRCPEP | 6 |
| | | KSCDTPPPCPRCPEPKSCD | |
| | | TPPPCPRCPEPKSCDTPPP | |
| | | CPRCP | |
| H7 | Human IgG4 | ESKYGPPCPSCP | 7 |
| H8 | Human IgG4(P) | ESKYGPPCPPCP | 8 |
| H9 | Engineered v1 | CPPC | 9 |
| H10 | Engineered v2 | CPSC | 10 |
| H11 | Engineered v3 | CPRC | 11 |
| H12 | Engineered v4 | SPPC | 12 |
| H13 | Engineered v5 | CPPS | 13 |
| H14 | Engineered v6 | SPPS | 14 |
| H15 | Engineered v7 | DKTHTCAA | 15 |
| H16 | Engineered v8 | DKTHTCPPCPA | 16 |
| H17 | Engineered v9 | DKTHTCPPCPATCPPCPA | 17 |
| H18 | Engineered v10 | DKTHTCPPCPATCPPCP | 18 |
| | | ATCPPCPA | |
| H19 | Engineered v11 | DKTHTCPPCPAGKPTLY | 19 |
| | | NSLVMSDTAGTCY | |
| H20 | Engineered v12 | DKTHTCPPCPAGKPTHV | 20 |
| | | NVSVVMAEVDGTCY | |
| H21 | Engineered v13 | DKTHTCCVECPPCPA | 21 |
| H22 | Engineered v14 | DKTHTCPRCPEPKSCDT | 22 |
| | | PPPCPRCPA | |
| H23 | Engineered v15 | DKTHTCPSCPA | 23 |

In one embodiment, the heavy chain Fc region possesses an intact hinge region at its N-terminus.

In one embodiment the heavy chain Fc region and hinge region are derived from IgG4 and the hinge region comprises the modified sequence CPPC (SEQ ID NO: 9). The core hinge region of human IgG4 contains the sequence CPSC (SEQ ID NO: 728) compared to IgG1 which contains the sequence CPPC (SEQ ID NO: 729). The serine residue present in the IgG4 sequence leads to increased flexibility in this region, and therefore a proportion of molecules form disulfide bonds within the same protein chain (an intrachain disulfide) rather than bridging to the other heavy chain in the IgG molecule to form the interchain disulfide. (Angel et al., 1993, Mol Immunol 30(1):105-108). Changing the serine residue to a proline to give the same core sequence as IgG1 allows complete formation of inter-chain disulfides in the IgG4 hinge region, thus reducing heterogeneity in the purified product. This altered isotype is termed IgG4P.

### 7.3.3. ABM Linkers

In certain aspects, the present disclosure provides MBMs (e.g., TBMs) comprising at least three ABMs, where two or more components of an ABM (e.g., a VH and a VL of an scFv), two or more ABMs, or an ABM and a non-ABM domain (e.g., a dimerization domain such as an Fc region) are connected to one another by a peptide linker. Such linkers are referred to herein an “ABM linkers”, as opposed to the ADC linkers used to attach drugs to MBMs as described, for example, in Section 7.8.2.

A peptide linker can range from 2 amino acids to 60 or more amino acids, and in certain aspects a peptide linker ranges from 3 amino acids to 50 amino acids, from 4 to 30 amino acids, from 5 to 25 amino acids, from 10 to 25 amino acids or from 12 to 20 amino acids. In particular embodiments, a peptide linker is 2 amino acids, 3 amino acids, 4 amino acid, 5 amino acids, 6 amino acids, 7 amino acids, 8 amino acids, 9 amino acids, 10 amino acids, 11 amino acids, 12 amino acids, 13 amino acids, 14 amino acid, 15 amino acids, 16 amino acids, 17 amino acids, 18 amino acids, 19 amino acids, 20 amino acids, 21 amino acids, 22 amino acids, 23 amino acids, 24 amino acid, 25 amino acids, 26 amino acids, 27 amino acids, 28 amino acids, 29 amino acids, 30 amino acids, 31 amino acids, 32 amino acids, 33 amino acids, 34 amino acid, 35 amino acids, 36 amino acids, 37 amino acids, 38 amino acids, 39 amino acids, 40 amino acids, 41 amino acids, 42 amino acids, 43 amino acids, 44 amino acid, 45 amino acids, 46 amino acids, 47 amino acids, 48 amino acids, 49 amino acids, or 50 amino acids in length.

Charged and/or flexible linkers can be used.

Examples of flexible ABM linkers that can be used in the MBMs of the disclosure include those disclosed by Chen et al., 2013, Adv Drug Deliv Rev. 65(10):1357-1369 and Klein et al., 2014, Protein Engineering, Design & Selection 27(10):325-330. A particularly useful flexible linker is (GGGGS)n (also referred to as (G4S)n) (SEQ ID NO: 44). In some embodiments, n is any number between 1 and 10, i.e., 1, 2, 3, 4, 5, 6, 7, 8, 9, and 10, or any range bounded by any two of the foregoing numbers, e.g., 1 to 5, 2 to 5, 3 to 6, 2 to 4, 1 to 4, and so on and so forth.

Other examples of suitable ABM linkers for use in the MBMs of the present disclosure are shown in Table 4 below:

TABLE 4
ABM Linker Sequences

| Linker | | SEQ ID |
|---|---|---|
| Name | Linker Sequence | NO: |
| L1 | ADAAP | 24 |
| L2 | ADAAPTVSIFP | 25 |
| L3 | ADAAPTVSIFPP | 26 |
| L4 | AKTTAP | 27 |
| L5 | AKTTAPSVYPLAP | 28 |
| L6 | AKTTPKLEEGEFSEARV | 29 |
| L7 | AKTTPKLGG | 30 |
| L8 | AKTTPP | 31 |
| L9 | AKTTPPSVTPLAP | 32 |
| L10 | ASTKGP | 33 |
| L11 | ASTKGPSVFPLAP | 34 |
| L12 | ASTKGPSVFPLAP | 35 |
| | ASTKGPSVFPLAP | |
| L13 | EGKSSGSGSESKST | 36 |
| L14 | GEGESGEGESGEGES | 37 |
| L15 | GEGESGEGESGEGESGEGES | 38 |
| L16 | GEGGSGEGGSGEGGS | 39 |
| L17 | GENKVEYAPALMALS | 40 |
| L18 | GGEGSGGEGSGGEGS | 41 |
| L19 | GGGESGGEGSGEGGS | 42 |
| L20 | GGGESGGGESGGGES | 43 |
| L21 | (GGGGS)ₙ, (also | 44 |
| | referred to as | |
| | (G4S)ₙ), where n | |
| | can be 1-10. | |
| L22 | GGGGSGGGGS | 45 |
| L23 | GGGGSGGGGSGGGGS | 46 |
| L24 | GGGGSGGGGSGGGGSGGGGS | 47 |
| L25 | GGGKSGGGKSGGGKS | 48 |
| L26 | GGGKSGGKGSGKGGS | 49 |
| L27 | GGKGSGGKGSGGKGS | 50 |
| L28 | GGSGG | 51 |
| L29 | GGSGGGGSG | 52 |
| L30 | GGSGGGGSGGGGS | 53 |
| L31 | GHEAAAVMQVQYPAS | 54 |
| L32 | GKGGSGKGGSGKGGS | 55 |
| L33 | GKGKSGKGKSGKGKS | 56 |
| L34 | GKGKSGKGKSGKGKSGKGKS | 57 |
| L35 | GKPGSGKPGSGKPGS | 58 |
| L36 | GKPGSGKPGSGKPGSGKPGS | 59 |
| L37 | GPAKELTPLKEAKVS | 60 |
| L38 | GSAGSAAGSGEF | 61 |
| L39 | IRPRAIGGSKPRVA | 62 |
| L40 | KESGSVSSEQLAQFRSLD | 63 |
| L41 | KTTPKLEEGEFSEAR | 64 |
| L42 | QPKAAP | 65 |
| L43 | QPKAAPSVTLFPP | 66 |
| L44 | RADAAAA(G4S)₄ | 67 |
| L45 | RADAAAAGGPGS | 68 |
| L46 | RADAAP | 69 |
| L47 | RADAAPTVS | 70 |
| L48 | SAKTTP | 71 |
| L49 | SAKTTPKLEEGEFSEARV | 72 |
| L50 | SAKTTPKLGG | 73 |
| L51 | STAGDTHLGGEDFD | 74 |
| L52 | TVAAP | 75 |
| L53 | TVAAPSVFIFPP | 76 |
| L54 | TVAAPSVFIFPPTV | 77 |
| | AAPSVFIFPP | |

In various aspects, the disclosure provides a MBM (e.g., a TBM) which comprises one or more ABM linkers. Each of the ABM linkers can be range from 2 amino acids to 60 amino acids in length, e.g., 4 to 30 amino acids, from 5 to 25 amino acids, from 10 to 25 amino acids or from 12 to 20 amino acids in length, optionally selected from Table 4 above. In particular embodiments, the MBM comprises two, three, four, five or six ABM linkers. The ABM linkers can be on one, two, three, four or even more polypeptide chains of the MBM.

### 7.4. Exemplary Trispecific Binding Molecules

Exemplary TBM configurations are shown in FIG. 1. FIG. 1A shows the components of the TBM configurations shown in FIGS. 1B-1U. The scFv, Fab, non-immunoglobulin based ABM, and Fc each can have the characteristics described for these components in Sections 7.2 and 7.3. The components of the TBM configurations shown in FIG. 1 can be associated with each other by any of the means described in Sections 7.2 and 7.3 (e.g., by direct bonds, ABM linkers, disulfide bonds, Fc domains with modified with knob in hole interactions, etc.). The orientations and associations of the various components shown in FIG. 1 are merely exemplary; as will be appreciated by skilled artisans, other orientations and associations may be suitable (e.g., as described in Sections 7.2 and 7.3).

TBMs of the disclosure are not limited to the configurations shown in FIG. 1. Other configurations that may be used are known to those skilled in the art. See, e.g., WO 2014/145806; WO 2017/124002; Liu et al., 2017, Front Immunol. 8:38; Brinkmann & Kontermann, 2017, mAbs 9:2, 182-212; US 2016/0355600; Klein et al., 2016, MAbs 8(6):1010-20; and US 2017/0145116.

### 7.4.1. Exemplary Trivalent TBMs

The TBMs of the disclosure can be trivalent, i.e., they have three antigen-binding domains, each of which binds TAA 1, TAA 2, or a component of a TCR complex.

Exemplary trivalent TBM configurations are shown in FIGS. 1B through 10.

As depicted in FIGS. 1B-1K, a TBM can comprise two half antibodies, one comprising two ABMs and the other comprising one ABM, the two halves paired through an Fc domain.

In the embodiment of FIG. 1B, the first (or left) half antibody comprises an scFv and an Fc region, and the second (or right) half antibody comprises a Fab, an scFv and an Fc region. The first and second half antibodies are associated through the Fc regions forming an Fc domain.

In the embodiment of FIG. 1C, the first (or left) half antibody comprises two Fab and an Fc region, and the second (or right) half antibody comprises a Fab and an Fc region. The first and second half antibodies are associated through the Fc regions forming an Fc domain.

In the embodiment of FIG. 1D, the first (or left) half antibody comprises a Fab, an scFv and an Fc region, and the second (or right) half antibody comprises a Fab and an Fc region. The first and second half antibodies are associated through the Fc regions forming an Fc domain.

In the embodiment of FIG. 1E, the first (or left) half antibody comprises an scFv and an Fc region, and the second (or right) half antibody comprises two Fab and an Fc region. The first and second half antibodies are associated through the Fc regions forming an Fc domain.

In the embodiment of FIG. 1F, the first (or left) half antibody comprises an scFv, an Fc region, and a Fab, and the second (or right) half antibody comprises a Fab and an Fc region. The first and second half antibodies are associated through the Fc regions forming an Fc domain.

In the embodiment of FIG. 1G, the first (or left) half antibody comprises an scFv and an Fc region, and the second (or right) half antibody comprises a Fab an Fc region, and an scFV. The first and second half antibodies are associated through the Fc regions forming an Fc domain.

In the embodiment of FIG. 1H, the first (or left) half antibody comprises two Fab and an Fc region, and the second (or right) half antibody comprises a non-immunoglobulin based ABM and an Fc region. The first and second half antibodies are associated through the Fc regions forming an Fc domain.

In the embodiment of FIG. 1I, the first (or left) half antibody comprises a Fab, an scFv, and an Fc region, and the second (or right) half antibody comprises a non-immunoglobulin based ABM and an Fc region. The first and second half antibodies are associated through the Fc regions forming an Fc domain.

In the embodiment of FIG. 1J, the first (or left) half antibody comprises a Fab and an Fc region, and the second (or right) half antibody comprises an scFv, a non-immunoglobulin based ABM and an Fc region. The first and second half antibodies are associated through the Fc regions forming an Fc domain.

In the embodiment of FIG. 1K, the first (or left) half antibody comprises an scFv and an Fc region, and the second (or right) half antibody comprises an scFv, an Fc region, and a second scFv. The first and second half antibodies are associated through the Fc regions forming an Fc domain.

In the embodiment of FIG. 1N, the first (or left) half antibody comprises a Fab, an Fc region, and an scFv, and the second (or right) half antibody comprises a Fab, and an Fc region. The first and second half antibodies are associated through the Fc regions forming an Fc domain.

In the embodiment of FIG. 1O, the first (or left) half antibody comprises a Fab, an Fc region, and a scFab, and the second (or right) half antibody comprises a Fab and an Fc region. The first and second half antibodies are associated through the Fc regions forming an Fc domain.

Alternatively, as depicted in FIG. 1L, trivalent a TBM can comprise two half antibodies, each comprising one complete ABM and a portion of another ABM (one a VH, the other a VL). The two half antibodies are paired through an Fc domain, whereupon the VH and the VL associate to form a complete antigen-binding Fv domain.

The TBM can be a single chain, as shown in FIG. 1M. The TBM of FIG. 1M comprises three scFv domains connected through linkers.

In each of the configurations shown in FIGS. 1B-10, each of the domains designated X, Y, and Z represents a TCR ABM, a TAA 1 ABM, or a TAA 2 ABM, although not necessarily in that order. In order words, X can be a TCR ABM, a TAA 1 ABM, or TAA 2 ABM, Y can be a TCR ABM, a TAA 1 ABM, or a TAA 2 ABM, and Z can be a TAA 1 ABM, a TCR ABM, or a TAA 2 ABM, provided that the TBM comprises at least one TCR ABM, at least one TAA 1 ABM, and at least one TAA 2 ABM.

Accordingly, in the present disclosure provides a trivalent TBM as shown in any one of FIGS. 1B through 10, where X is a TAA 1 ABM, Y is a TCR ABM and Z is a TAA 2 ABM (this configuration of ABMs designated as “T1” for convenience).

The present disclosure also provides a trivalent TBM as shown in any one of FIGS. 1B through 10, where X is a TAA 1 ABM, Y is a TAA 2 ABM, and Z is a TCR ABM (this configuration of ABMs designated as “T2” for convenience).

The present disclosure further provides a trivalent TBM as shown in any one of FIGS. 1B through 10, where X is a TCR ABM, Y is a TAA 1 ABM, and Z is a TAA 2 ABM (this configuration of ABMs designated as “T3” for convenience).

The present disclosure yet further provides a trivalent TBM as shown in any one of FIGS. 1B through 10, where X is a TCR ABM, Y is a TAA 2 ABM, and Z is a TAA 1 ABM (this configuration of ABMs designated as “T4” for convenience).

The present disclosure yet further provides a trivalent TBM as shown in any one of FIGS. 1B through 10, where X is a TAA 2 ABM, Y is a TAA 1 ABM, and Z is a TCR ABM (this configuration of ABMs designated as “T5” for convenience).

The present disclosure yet further provides a trivalent TBM as shown in any one of FIGS. 1B through 10, where X is a TAA 2 ABM, Y is a TCR ABM, and Z is a TAA 1 ABM (this configuration of ABMs designated as “T6” for convenience).

### 7.4.2. Exemplary Tetravalent TBMs

The TBMs of the disclosure can be tetravalent, i.e., they have four antigen-binding domains, one or two of which binds TAA 1, one or two of which binds TAA 2, and one or two of which binds a component of a TCR complex.

Exemplary tetravalent TBM configurations are shown in FIGS. 1P-1R.

As depicted in FIGS. 1P-1R, a tetravalent TBM can comprise two half antibodies, each comprising two complete ABMs, the two halves paired through an Fc domain.

In the embodiment of FIG. 1P, the first (or left) half antibody comprises a Fab, an Fc region, and a second Fab, and the second (or right) half antibody comprises a Fab, an Fc region, and a second Fab. The first and second half antibodies are associated through the Fc regions forming an Fc domain.

In the embodiment of FIG. 1Q, the first (or left) half antibody comprises a Fab, an Fc region, and an scFv, and the second (or right) half antibody comprises a Fab, an Fc region, and an scFv. The first and second half antibodies are associated through the Fc regions forming an Fc domain.

In the embodiment of FIG. 1R, the first (or left) half antibody comprises a Fab, an Fc region, and an scFv, and the second (or right) half antibody comprises an scFv, an Fc region, and a Fab. The first and second half antibodies are associated through the Fc regions forming an Fc domain.

In the configuration shown in FIGS. 1P-1R, each of X, Y, Z, and A represent a TCR ABM, a TAA 1 ABM, or a TAA 2 ABM, although not necessarily in that order, and provided that the TBM comprises at least one TCR ABM, one TAA 1 ABM, and one TAA 2 ABM. Thus, the tetravalent ABMs of the disclosure will include two ABMs against one of TAA 1, TAA 2, and a component of a TCR complex. In some cases, a tetravalent TBM has two TAA 1 or TAA 2 ABMs.

Accordingly, in the present disclosure provides tetravalent TBMs as shown in any one of FIGS. 1P-1R, where X, Y, Z, and A are ABMs directed to TAA 1, TAA 2 and a component of a TCR complex, as shown in Table 5.

TABLE 5
ABM Permutations in Tetravalent TBMs

| Tetravalent | | | | |
|---|---|---|---|---|
| Configuration | X | Y | Z | A |
| Tv 1 | TAA 1 | TAA 1 | TAA 2 | TCR |
| Tv 2 | TAA 1 | TAA 1 | TCR | TAA 2 |
| Tv 3 | TAA 1 | TAA 2 | TAA 1 | TCR |
| Tv 4 | TAA 1 | TCR | TAA 1 | TAA 2 |
| Tv 5 | TAA 1 | TAA 2 | TCR | TAA 1 |
| Tv 6 | TAA 1 | TCR | TAA 2 | TAA 1 |
| Tv 7 | TAA 2 | TAA 1 | TAA 1 | TCR |
| Tv 8 | TCR | TAA 1 | TAA 1 | TAA 2 |
| Tv 9 | TAA 2 | TAA 1 | TCR | TAA 1 |
| Tv 10 | TCR | TAA 1 | TAA 2 | TAA 1 |
| Tv 11 | TAA 2 | TCR | TAA 1 | TAA 1 |
| Tv 12 | TCR | TAA 2 | TAA 1 | TAA 1 |
| Tv 13 | TAA 1 | TAA 2 | TCR | TCR |
| Tv 14 | TAA 1 | TCR | TAA 2 | TCR |
| Tv 15 | TAA 1 | TCR | TCR | TAA 2 |
| Tv 16 | TAA 2 | TAA 1 | TCR | TCR |
| Tv 17 | TCR | TAA 1 | TAA 2 | TCR |
| Tv 18 | TCR | TAA 1 | TCR | TAA 2 |
| Tv 19 | TAA 2 | TCR | TAA 1 | TCR |
| Tv 20 | TCR | TAA 2 | TAA 1 | TCR |
| Tv 21 | TCR | TCR | TAA 1 | TAA 2 |
| Tv 22 | TAA 2 | TCR | TCR | TAA 1 |
| Tv 23 | TCR | TAA 2 | TCR | TAA 1 |
| Tv 24 | TCR | TCR | TAA 2 | TAA 1 |

### 7.4.3. Exemplary Pentavalent TBMs

The TBMs of the disclosure can be pentavalent, i.e., they have five antigen-binding domains, one, two, or three of which binds TAA 1, one, two, or three of which binds TAA 2, and one, two, or three of which binds a component of a TCR complex.

An exemplary pentavalent TBM configuration is shown in FIG. 1S.

As depicted in FIG. 1S, a pentavalent TBM can comprise two half antibodies, one of which comprises two complete ABMs and the other of which comprises one complete ABM, the two halves paired through an Fc domain.

In the embodiment of FIG. 1S, the first (or left) half antibody comprises a Fab, an scFv, and an Fc region, and the second (or right) half antibody comprises a Fab, an Fc region, and an scFv. The first and second half antibodies are associated through the Fc regions forming an Fc domain.

In the configuration shown in FIG. 1S, each of X, Y, Z, A, and B represent a TCR ABM, a TAA 1 ABM, or a TAA 2 ABM, although not necessarily in that order, and provided that the TBM comprises at least one TCR ABM, one TAA 1 ABM, and one TAA 2 ABM. Thus, the pentavalent TBMs of the disclosure can include two ABMs against two of TAA 1, TAA 2, and a component of a TCR complex, or three ABMs against one of TAA 1, TAA 2, and a component of a TCR complex. In some cases, a pentavalent TBM has two or three TAA 1 or TAA 2 ABMs. In a specific embodiment, a pentavalent TBM has three TAA 1 ABMs, one TAA 2 ABM and one TCR ABM. In another embodiment, a pentavalent TBM has two TAA 1 ABMs, two TAA 2 ABMs, and one TCR ABM.

Accordingly, the present disclosure provides a pentavalent TBM as shown in FIG. 1S, where X, Y, Z, A, and B are ABMs directed to TAA 1, TAA 2, and a component of a TCR complex as shown in Table 6.

TABLE 6
ABM Permutations in Pentavalent TBMs

| Pentavalent | | | | | |
|---|---|---|---|---|---|
| Configuration | X | Y | Z | A | B |
| Pv 1 | TAA 1 | TAA 1 | TAA 1 | TAA 2 | TCR |
| Pv 2 | TAA 1 | TAA 1 | TAA 1 | TCR | TAA 2 |
| Pv 3 | TAA 1 | TAA 1 | TAA 2 | TAA 1 | TCR |
| Pv 4 | TAA 1 | TAA 1 | TCR | TAA 1 | TAA 2 |
| Pv 5 | TAA 1 | TAA 1 | TAA 2 | TCR | TAA 1 |
| Pv 6 | TAA 1 | TAA 1 | TCR | TAA 2 | TAA 1 |
| Pv 7 | TAA 1 | TAA 2 | TAA 1 | TAA 1 | TCR |
| Pv 8 | TAA 1 | TCR | TAA 1 | TAA 1 | TAA 2 |
| Pv 9 | TAA 1 | TAA 2 | TAA 1 | TCR | TAA 1 |
| Pv 10 | TAA 1 | TCR | TAA 1 | TAA 2 | TAA 1 |
| Pv 11 | TAA 1 | TAA 2 | TCR | TAA 1 | TAA 1 |
| Pv 12 | TAA 1 | TCR | TAA 2 | TAA 1 | TAA 1 |
| Pv 13 | TAA 2 | TAA 1 | TAA 1 | TAA 1 | TCR |
| Pv 14 | TCR | TAA 1 | TAA 1 | TAA 1 | TAA 2 |
| Pv 15 | TAA 2 | TAA 1 | TAA 1 | TCR | TAA 1 |
| Pv 16 | TCR | TAA 1 | TAA 1 | TAA 2 | TAA 1 |
| Pv 17 | TAA 2 | TAA 1 | TCR | TAA 1 | TAA 1 |
| Pv 18 | TCR | TAA 1 | TAA 2 | TAA 1 | TAA 1 |
| Pv 19 | TAA 2 | TCR | TAA 1 | TAA 1 | TAA 1 |
| Pv 20 | TCR | TAA 2 | TAA 1 | TAA 1 | TAA 1 |
| Pv 21 | TAA 1 | TAA 1 | TAA 2 | TAA 2 | TCR |
| Pv 22 | TAA 1 | TAA 1 | TAA 2 | TCR | TAA 2 |
| Pv 23 | TAA 1 | TAA 1 | TCR | TAA 2 | TAA 2 |
| Pv 24 | TAA 1 | TAA 2 | TAA 1 | TAA 2 | TCR |
| Pv 25 | TAA 1 | TAA 2 | TAA 1 | TCR | TAA 2 |
| Pv 26 | TAA 1 | TCR | TAA 1 | TAA 2 | TAA 2 |
| Pv 27 | TAA 1 | TAA 2 | TAA 2 | TAA 1 | TCR |
| Pv 28 | TAA 1 | TAA 2 | TCR | TAA 1 | TAA 2 |
| Pv 29 | TAA 1 | TCR | TAA 2 | TAA 1 | TAA 2 |
| Pv 30 | TAA 1 | TAA 2 | TAA 2 | TCR | TAA 1 |
| Pv 31 | TAA 1 | TAA 2 | TCR | TAA 2 | TAA 1 |
| Pv 32 | TAA 1 | TCR | TAA 2 | TAA 2 | TAA 1 |
| Pv 33 | TAA 2 | TAA 1 | TAA 1 | TAA 2 | TCR |
| Pv 34 | TAA 2 | TAA 1 | TAA 1 | TCR | TAA 2 |
| Pv 35 | TCR | TAA 1 | TAA 1 | TAA 2 | TAA 2 |
| Pv 36 | TAA 2 | TAA 1 | TAA 2 | TAA 1 | TCR |
| Pv 37 | TAA 2 | TAA 1 | TCR | TAA 1 | TAA 2 |
| Pv 38 | TCR | TAA 1 | TAA 2 | TAA 1 | TAA 2 |
| Pv 39 | TAA 2 | TAA 1 | TAA 2 | TCR | TAA 1 |
| Pv 40 | TAA 2 | TAA 1 | TCR | TAA 2 | TAA 1 |
| Pv 41 | TCR | TAA 1 | TAA 2 | TAA 2 | TAA 1 |
| Pv 42 | TAA 2 | TAA 2 | TAA 1 | TAA 1 | TCR |
| Pv 43 | TAA 2 | TCR | TAA 1 | TAA 1 | TAA 2 |
| Pv 44 | TCR | TAA 2 | TAA 1 | TAA 1 | TAA 2 |
| Pv 45 | TAA 2 | TAA 2 | TAA 1 | TCR | TAA 1 |
| Pv 46 | TAA 2 | TCR | TAA 1 | TAA 2 | TAA 1 |
| Pv 47 | TCR | TAA 2 | TAA 1 | TAA 2 | TAA 1 |
| Pv 48 | TAA 2 | TAA 2 | TCR | TAA 1 | TAA 1 |
| Pv 49 | TAA 2 | TCR | TAA 2 | TAA 1 | TAA 1 |
| Pv 50 | TCR | TAA 2 | TAA 2 | TAA 1 | TAA 1 |
| Pv 51 | TAA 1 | TAA 1 | TAA 2 | TCR | TCR |
| Pv 52 | TAA 1 | TAA 1 | TCR | TAA 2 | TCR |
| Pv 53 | TAA 1 | TAA 1 | TCR | TCR | TAA 2 |
| Pv 54 | TAA 1 | TAA 2 | TAA 1 | TCR | TCR |
| Pv 55 | TAA 1 | TCR | TAA 1 | TAA 2 | TCR |
| Pv 56 | TAA 1 | TCR | TAA 1 | TCR | TAA 2 |
| Pv 57 | TAA 1 | TAA 2 | TCR | TAA 1 | TCR |
| Pv 58 | TAA 1 | TCR | TAA 2 | TAA 1 | TCR |
| Pv 59 | TAA 1 | TCR | TCR | TAA 1 | TAA 2 |
| Pv 60 | TAA 1 | TAA 2 | TCR | TCR | TAA 1 |
| Pv 61 | TAA 1 | TCR | TAA 2 | TCR | TAA 1 |
| Pv 62 | TAA 1 | TCR | TCR | TAA 2 | TAA 1 |
| Pv 63 | TAA 2 | TAA 1 | TAA 1 | TCR | TCR |
| Pv 64 | TCR | TAA 1 | TAA 1 | TAA 2 | TCR |
| Pv 65 | TCR | TAA 1 | TAA 1 | TCR | TAA 2 |
| Pv 66 | TAA 2 | TAA 1 | TCR | TAA 1 | TCR |
| Pv 67 | TCR | TAA 1 | TAA 2 | TAA 1 | TCR |
| Pv 68 | TCR | TAA 1 | TCR | TAA 1 | TAA 2 |
| Pv 69 | TAA 2 | TAA 1 | TCR | TCR | TAA 1 |
| Pv 70 | TCR | TAA 1 | TAA 2 | TCR | TAA 1 |
| Pv 71 | TCR | TAA 1 | TCR | TAA 2 | TAA 1 |
| Pv 72 | TAA 2 | TCR | TAA 1 | TAA 1 | TCR |
| Pv 73 | TCR | TAA 2 | TAA 1 | TAA 1 | TCR |
| Pv 74 | TCR | TCR | TAA 1 | TAA 1 | TAA 2 |
| Pv 75 | TAA 2 | TCR | TAA 1 | TCR | TAA 1 |
| Pv 76 | TCR | TAA 2 | TAA 1 | TCR | TAA 1 |
| Pv 77 | TCR | TCR | TAA 1 | TAA 2 | TAA 1 |
| Pv 78 | TAA 2 | TCR | TCR | TAA 1 | TAA 1 |
| Pv 79 | TCR | TAA 2 | TCR | TAA 1 | TAA 1 |
| Pv 80 | TCR | TCR | TAA 2 | TAA 1 | TAA 1 |
| Pv 81 | TAA 1 | TAA 2 | TCR | TCR | TCR |
| Pv 82 | TAA 1 | TCR | TAA 2 | TCR | TCR |
| Pv 83 | TAA 1 | TCR | TCR | TAA 2 | TCR |
| Pv 84 | TAA 1 | TCR | TCR | TCR | TAA 2 |
| Pv 85 | TAA 2 | TAA 1 | TCR | TCR | TCR |
| Pv 86 | TCR | TAA 1 | TAA 2 | TCR | TCR |
| Pv 87 | TCR | TAA 1 | TCR | TAA 2 | TCR |
| Pv 88 | TCR | TAA 1 | TCR | TCR | TAA 2 |
| Pv 89 | TAA 2 | TCR | TAA 1 | TCR | TCR |
| Pv 90 | TCR | TAA 2 | TAA 1 | TCR | TCR |
| Pv 91 | TCR | TCR | TAA 1 | TAA 2 | TCR |
| Pv 92 | TCR | TCR | TAA 1 | TCR | TAA 2 |
| Pv 93 | TAA 2 | TCR | TCR | TAA 1 | TCR |
| Pv 94 | TCR | TAA 2 | TCR | TAA 1 | TCR |
| Pv 95 | TCR | TCR | TAA 2 | TAA 1 | TCR |
| Pv 96 | TCR | TCR | TCR | TAA 1 | TAA 2 |
| Pv 97 | TAA 2 | TCR | TCR | TCR | TAA 1 |
| Pv 98 | TCR | TAA 2 | TCR | TCR | TAA 1 |
| Pv 99 | TCR | TCR | TAA 2 | TCR | TAA 1 |
| Pv 100 | TCR | TCR | TCR | TAA 2 | TAA 1 |

### 7.4.4. Exemplary Hexavalent TBMs

The TBMs of the disclosure can be hexavalent, i.e., they have six antigen-binding domains, one, two, three, or four of which binds TAA 1, one, two, three, or four of which binds TAA 2, and one, two, three, or four of which binds a component of a TCR complex.

Exemplary hexavalent TBM configurations are shown in FIGS. 1T-1U.

As depicted in FIGS. 1T-1U, a pentavalent TBM can comprise two half antibodies, one of which comprises two complete ABMs and the other of which comprises one complete ABM, the two halves paired through an Fc domain.

In the embodiment of FIG. 1T, the first (or left) half antibody comprises a Fab, a second Fab, an Fc region, and an scFv, and the second (or right) half antibody comprises a Fab, a second Fab, an Fc region, and an scFv. The first and second half antibodies are associated through the Fc regions forming an Fc domain.

In the embodiment of FIG. 1U, the first (or left) half antibody comprises a first Fv, a second Fv, a third Fv, and an Fc region, and the second (or right) half antibody comprises a first Fv, a second Fv, a third Fv, and an Fc region. The first and second half antibodies are associated through the Fc regions forming an Fc domain.

In the configuration shown in FIGS. 1T-1U, each of X, Y, Z, A, B, and C represent a TCR ABM, a TAA 1 ABM, or a TAA 2 ABM, although not necessarily in that order, and provided that the TBM comprises at least one TCR ABM, one TAA 1 ABM, and one TAA 2 ABM. Thus, the hexavalent TBMs of the disclosure can include (i) two ABMs against each of TAA 1, TAA 2, and a component of a TCR complex, (ii) three ABMs against one of a component of a TAA 1, TAA 2, and a component of a TCR complex, or (iii) four ABMs against one of TAA 1, TAA 2, and a component of a TBR complex. For example, a hexavalent ABM can include three ABMs against TAA 1, two ABMs against TAA 2 and one ABM against a component of a TR complex. As another example, a hexavalent ABM can include three ABMs against TAA 1, two ABMs against a component of a TCR complex and one ABM against TAA 2. In some cases, a hexavalent TBM has two, three, our four TAA 1 or TAA 2 ABMs. In a specific embodiment, a hexavalent TBM has three TAA 1 or TAA 2 ABMs. In other embodiments, a hexavalent TBM has four TAA 1 or TAA 2 ABMs.

Accordingly, in the present disclosure provides hexavalent TBMs as shown in any one of FIGS. 1T-1U, where X, Y, Z, A, B, and C are ABMs directed to TAA 1, TAA 2 and a component of a TCR complex, as shown in Table 7.

TABLE 7
ABM Permutations in Hexavalent TBMs

| Hexavalent | | | | | | |
|---|---|---|---|---|---|---|
| Configuration | X | Y | Z | A | B | C |
| Hv 1 | TAA 1 | TAA 1 | TAA 1 | TAA 1 | TAA 2 | TCR |
| Hv 2 | TAA 1 | TAA 1 | TAA 1 | TAA 1 | TCR | TAA 2 |
| Hv 3 | TAA 1 | TAA 1 | TAA 1 | TAA 2 | TAA 1 | TCR |
| Hv 4 | TAA 1 | TAA 1 | TAA 1 | TCR | TAA 1 | TAA 2 |
| Hv 5 | TAA 1 | TAA 1 | TAA 1 | TAA 2 | TCR | TAA 1 |
| Hv 6 | TAA 1 | TAA 1 | TAA 1 | TCR | TAA 2 | TAA 1 |
| Hv 7 | TAA 1 | TAA 1 | TAA 2 | TAA 1 | TAA 1 | TCR |
| Hv 8 | TAA 1 | TAA 1 | TCR | TAA 1 | TAA 1 | TAA 2 |
| Hv 9 | TAA 1 | TAA 1 | TAA 2 | TAA 1 | TCR | TAA 1 |
| Hv 10 | TAA 1 | TAA 1 | TCR | TAA 1 | TAA 2 | TAA 1 |
| Hv 11 | TAA 1 | TAA 1 | TAA 2 | TCR | TAA 1 | TAA 1 |
| Hv 12 | TAA 1 | TAA 1 | TCR | TAA 2 | TAA 1 | TAA 1 |
| Hv 13 | TAA 1 | TAA 2 | TAA 1 | TAA 1 | TAA 1 | TCR |
| Hv 14 | TAA 1 | TCR | TAA 1 | TAA 1 | TAA 1 | TAA 2 |
| Hv 15 | TAA 1 | TAA 2 | TAA 1 | TAA 1 | TCR | TAA 1 |
| Hv 16 | TAA 1 | TCR | TAA 1 | TAA 1 | TAA 2 | TAA 1 |
| Hv 17 | TAA 1 | TAA 2 | TAA 1 | TCR | TAA 1 | TAA 1 |
| Hv 18 | TAA 1 | TCR | TAA 1 | TAA 2 | TAA 1 | TAA 1 |
| Hv 19 | TAA 1 | TAA 2 | TCR | TAA 1 | TAA 1 | TAA 1 |
| Hv 20 | TAA 1 | TCR | TAA 2 | TAA 1 | TAA 1 | TAA 1 |
| Hv 21 | TAA 2 | TAA 1 | TAA 1 | TAA 1 | TAA 1 | TCR |
| Hv 22 | TCR | TAA 1 | TAA 1 | TAA 1 | TAA 1 | TAA 2 |
| Hv 23 | TAA 2 | TAA 1 | TAA 1 | TAA 1 | TCR | TAA 1 |
| Hv 24 | TCR | TAA 1 | TAA 1 | TAA 1 | TAA 2 | TAA 1 |
| Hv 25 | TAA 2 | TAA 1 | TAA 1 | TCR | TAA 1 | TAA 1 |
| Hv 26 | TCR | TAA 1 | TAA 1 | TAA 2 | TAA 1 | TAA 1 |
| Hv 27 | TAA 2 | TAA 1 | TCR | TAA 1 | TAA 1 | TAA 1 |
| Hv 28 | TCR | TAA 1 | TAA 2 | TAA 1 | TAA 1 | TAA 1 |
| Hv 29 | TAA 2 | TCR | TAA 1 | TAA 1 | TAA 1 | TAA 1 |
| Hv 30 | TCR | TAA 2 | TAA 1 | TAA 1 | TAA 1 | TAA 1 |
| Hv 31 | TAA 1 | TAA 1 | TAA 1 | TAA 2 | TAA 2 | TCR |
| Hv 32 | TAA 1 | TAA 1 | TAA 1 | TAA 2 | TCR | TAA 2 |
| Hv 33 | TAA 1 | TAA 1 | TAA 1 | TCR | TAA 2 | TAA 2 |
| Hv 34 | TAA 1 | TAA 1 | TAA 2 | TAA 1 | TAA 2 | TCR |
| Hv 35 | TAA 1 | TAA 1 | TAA 2 | TAA 1 | TCR | TAA 2 |
| Hv 36 | TAA 1 | TAA 1 | TCR | TAA 1 | TAA 2 | TAA 2 |
| Hv 37 | TAA 1 | TAA 1 | TAA 2 | TAA 2 | TAA 1 | TCR |
| Hv 38 | TAA 1 | TAA 1 | TAA 2 | TCR | TAA 1 | TAA 2 |
| Hv 39 | TAA 1 | TAA 1 | TCR | TAA 2 | TAA 1 | TAA 2 |
| Hv 40 | TAA 1 | TAA 1 | TAA 2 | TAA 2 | TCR | TAA 1 |
| Hv 41 | TAA 1 | TAA 1 | TAA 2 | TCR | TAA 2 | TAA 1 |
| Hv 42 | TAA 1 | TAA 1 | TCR | TAA 2 | TAA 2 | TAA 1 |
| Hv 43 | TAA 1 | TAA 2 | TAA 1 | TAA 1 | TAA 2 | TCR |
| Hv 44 | TAA 1 | TAA 2 | TAA 1 | TAA 1 | TCR | TAA 2 |
| Hv 45 | TAA 1 | TCR | TAA 1 | TAA 1 | TAA 2 | TAA 2 |
| Hv 46 | TAA 1 | TAA 2 | TAA 1 | TAA 2 | TAA 1 | TCR |
| Hv 47 | TAA 1 | TAA 2 | TAA 1 | TCR | TAA 1 | TAA 2 |
| Hv 48 | TAA 1 | TCR | TAA 1 | TAA 2 | TAA 1 | TAA 2 |
| Hv 49 | TAA 1 | TAA 2 | TAA 1 | TAA 2 | TCR | TAA 1 |
| Hv 50 | TAA 1 | TAA 2 | TAA 1 | TCR | TAA 2 | TAA 1 |
| Hv 51 | TAA 1 | TCR | TAA 1 | TAA 2 | TAA 2 | TAA 1 |
| Hv 52 | TAA 1 | TAA 2 | TAA 2 | TAA 1 | TAA 1 | TCR |
| Hv 53 | TAA 1 | TAA 2 | TCR | TAA 1 | TAA 1 | TAA 2 |
| Hv 54 | TAA 1 | TCR | TAA 2 | TAA 1 | TAA 1 | TAA 2 |
| Hv 55 | TAA 1 | TAA 2 | TAA 2 | TAA 1 | TCR | TAA 1 |
| Hv 56 | TAA 1 | TAA 2 | TCR | TAA 1 | TAA 2 | TAA 1 |
| Hv 57 | TAA 1 | TCR | TAA 2 | TAA 1 | TAA 2 | TAA 1 |
| Hv 58 | TAA 1 | TAA 2 | TAA 2 | TCR | TAA 1 | TAA 1 |
| Hv 59 | TAA 1 | TAA 2 | TCR | TAA 2 | TAA 1 | TAA 1 |
| Hv 60 | TAA 1 | TCR | TAA 2 | TAA 2 | TAA 1 | TAA 1 |
| Hv 61 | TAA 2 | TAA 1 | TAA 1 | TAA 1 | TAA 2 | TCR |
| Hv 62 | TAA 2 | TAA 1 | TAA 1 | TAA 1 | TCR | TAA 2 |
| Hv 63 | TCR | TAA 1 | TAA 1 | TAA 1 | TAA 2 | TAA 2 |
| Hv 64 | TAA 2 | TAA 1 | TAA 1 | TAA 2 | TAA 1 | TCR |
| Hv 65 | TAA 2 | TAA 1 | TAA 1 | TCR | TAA 1 | TAA 2 |
| Hv 66 | TCR | TAA 1 | TAA 1 | TAA 2 | TAA 1 | TAA 2 |
| Hv 67 | TAA 2 | TAA 1 | TAA 1 | TAA 2 | TCR | TAA 1 |
| Hv 68 | TAA 2 | TAA 1 | TAA 1 | TCR | TAA 2 | TAA 1 |
| Hv 69 | TCR | TAA 1 | TAA 1 | TAA 2 | TAA 2 | TAA 1 |
| Hv 70 | TAA 2 | TAA 1 | TAA 2 | TAA 1 | TAA 1 | TCR |
| Hv 71 | TAA 2 | TAA 1 | TCR | TAA 1 | TAA 1 | TAA 2 |
| Hv 72 | TCR | TAA 1 | TAA 2 | TAA 1 | TAA 1 | TAA 2 |
| Hv 73 | TAA 2 | TAA 1 | TAA 2 | TAA 1 | TCR | TAA 1 |
| Hv 74 | TAA 2 | TAA 1 | TCR | TAA 1 | TAA 2 | TAA 1 |
| Hv 75 | TCR | TAA 1 | TAA 2 | TAA 1 | TAA 2 | TAA 1 |
| Hv 76 | TAA 2 | TAA 1 | TAA 2 | TCR | TAA 1 | TAA 1 |
| Hv 77 | TAA 2 | TAA 1 | TCR | TAA 2 | TAA 1 | TAA 1 |
| Hv 78 | TCR | TAA 1 | TAA 2 | TAA 2 | TAA 1 | TAA 1 |
| Hv 79 | TAA 2 | TAA 2 | TAA 1 | TAA 1 | TAA 1 | TCR |
| Hv 80 | TAA 2 | TCR | TAA 1 | TAA 1 | TAA 1 | TAA 2 |
| Hv 81 | TCR | TAA 2 | TAA 1 | TAA 1 | TAA 1 | TAA 2 |
| Hv 82 | TAA 2 | TAA 2 | TAA 1 | TAA 1 | TCR | TAA 1 |
| Hv 83 | TAA 2 | TCR | TAA 1 | TAA 1 | TAA 2 | TAA 1 |
| Hv 84 | TCR | TAA 2 | TAA 1 | TAA 1 | TAA 2 | TAA 1 |
| Hv 85 | TAA 2 | TAA 2 | TAA 1 | TCR | TAA 1 | TAA 1 |
| Hv 86 | TAA 2 | TCR | TAA 1 | TAA 2 | TAA 1 | TAA 1 |
| Hv 87 | TCR | TAA 2 | TAA 1 | TAA 2 | TAA 1 | TAA 1 |
| Hv 88 | TAA 2 | TAA 2 | TCR | TAA 1 | TAA 1 | TAA 1 |
| Hv 89 | TAA 2 | TCR | TAA 2 | TAA 1 | TAA 1 | TAA 1 |
| Hv 90 | TCR | TAA 2 | TAA 2 | TAA 1 | TAA 1 | TAA 1 |
| Hv 91 | TAA 1 | TAA 1 | TAA 1 | TAA 2 | TCR | TCR |
| Hv 92 | TAA 1 | TAA 1 | TAA 1 | TCR | TAA 2 | TCR |
| Hv 93 | TAA 1 | TAA 1 | TAA 1 | TCR | TCR | TAA 2 |
| Hv 94 | TAA 1 | TAA 1 | TAA 2 | TAA 1 | TCR | TCR |
| Hv 95 | TAA 1 | TAA 1 | TCR | TAA 1 | TAA 2 | TCR |
| Hv 96 | TAA 1 | TAA 1 | TCR | TAA 1 | TCR | TAA 2 |
| Hv 97 | TAA 1 | TAA 1 | TAA 2 | TCR | TAA 1 | TCR |
| Hv 98 | TAA 1 | TAA 1 | TCR | TAA 2 | TAA 1 | TCR |
| Hv 99 | TAA 1 | TAA 1 | TCR | TCR | TAA 1 | TAA 2 |
| Hv 100 | TAA 1 | TAA 1 | TAA 2 | TCR | TCR | TAA 1 |
| Hv 101 | TAA 1 | TAA 1 | TCR | TAA 2 | TCR | TAA 1 |
| Hv 102 | TAA 1 | TAA 1 | TCR | TCR | TAA 2 | TAA 1 |
| Hv 103 | TAA 1 | TAA 2 | TAA 1 | TAA 1 | TCR | TCR |
| Hv 104 | TAA 1 | TCR | TAA 1 | TAA 1 | TAA 2 | TCR |
| Hv 105 | TAA 1 | TCR | TAA 1 | TAA 1 | TCR | TAA 2 |
| Hv 106 | TAA 1 | TAA 2 | TAA 1 | TCR | TAA 1 | TCR |
| Hv 107 | TAA 1 | TCR | TAA 1 | TAA 2 | TAA 1 | TCR |
| Hv 108 | TAA 1 | TCR | TAA 1 | TCR | TAA 1 | TAA 2 |
| Hv 109 | TAA 1 | TAA 2 | TAA 1 | TCR | TCR | TAA 1 |
| Hv 110 | TAA 1 | TCR | TAA 1 | TAA 2 | TCR | TAA 1 |
| Hv 111 | TAA 1 | TCR | TAA 1 | TCR | TAA 2 | TAA 1 |
| Hv 112 | TAA 1 | TAA 2 | TCR | TAA 1 | TAA 1 | TCR |
| Hv 113 | TAA 1 | TCR | TAA 2 | TAA 1 | TAA 1 | TCR |
| Hv 114 | TAA 1 | TCR | TCR | TAA 1 | TAA 1 | TAA 2 |
| Hv 115 | TAA 1 | TAA 2 | TCR | TAA 1 | TCR | TAA 1 |
| Hv 116 | TAA 1 | TCR | TAA 2 | TAA 1 | TCR | TAA 1 |
| Hv 117 | TAA 1 | TCR | TCR | TAA 1 | TAA 2 | TAA 1 |
| Hv 118 | TAA 1 | TAA 2 | TCR | TCR | TAA 1 | TAA 1 |
| Hv 119 | TAA 1 | TCR | TAA 2 | TCR | TAA 1 | TAA 1 |
| Hv 120 | TAA 1 | TCR | TCR | TAA 2 | TAA 1 | TAA 1 |
| Hv 121 | TAA 2 | TAA 1 | TAA 1 | TAA 1 | TCR | TCR |
| Hv 122 | TCR | TAA 1 | TAA 1 | TAA 1 | TAA 2 | TCR |
| Hv 123 | TCR | TAA 1 | TAA 1 | TAA 1 | TCR | TAA 2 |
| Hv 124 | TAA 2 | TAA 1 | TAA 1 | TCR | TAA 1 | TCR |
| Hv 125 | TCR | TAA 1 | TAA 1 | TAA 2 | TAA 1 | TCR |
| Hv 126 | TCR | TAA 1 | TAA 1 | TCR | TAA 1 | TAA 2 |
| Hv 127 | TAA 2 | TAA 1 | TAA 1 | TCR | TCR | TAA 1 |
| Hv 128 | TCR | TAA 1 | TAA 1 | TAA 2 | TCR | TAA 1 |
| Hv 129 | TCR | TAA 1 | TAA 1 | TCR | TAA 2 | TAA 1 |
| Hv 130 | TAA 2 | TAA 1 | TCR | TAA 1 | TAA 1 | TCR |
| Hv 131 | TCR | TAA 1 | TAA 2 | TAA 1 | TAA 1 | TCR |
| Hv 132 | TCR | TAA 1 | TCR | TAA 1 | TAA 1 | TAA 2 |
| Hv 133 | TAA 2 | TAA 1 | TCR | TAA 1 | TCR | TAA 1 |
| Hv 134 | TCR | TAA 1 | TAA 2 | TAA 1 | TCR | TAA 1 |
| Hv 135 | TCR | TAA 1 | TCR | TAA 1 | TAA 2 | TAA 1 |
| Hv 136 | TAA 2 | TAA 1 | TCR | TCR | TAA 1 | TAA 1 |
| Hv 137 | TCR | TAA 1 | TAA 2 | TCR | TAA 1 | TAA 1 |
| Hv 138 | TCR | TAA 1 | TCR | TAA 2 | TAA 1 | TAA 1 |
| Hv 139 | TAA 2 | TCR | TAA 1 | TAA 1 | TAA 1 | TCR |
| Hv 140 | TCR | TAA 2 | TAA 1 | TAA 1 | TAA 1 | TCR |
| Hv 141 | TCR | TCR | TAA 1 | TAA 1 | TAA 1 | TAA 2 |
| Hv 142 | TAA 2 | TCR | TAA 1 | TAA 1 | TCR | TAA 1 |
| Hv 143 | TCR | TAA 2 | TAA 1 | TAA 1 | TCR | TAA 1 |
| Hv 144 | TCR | TCR | TAA 1 | TAA 1 | TAA 2 | TAA 1 |
| Hv 145 | TAA 2 | TCR | TAA 1 | TCR | TAA 1 | TAA 1 |
| Hv 146 | TCR | TAA 2 | TAA 1 | TCR | TAA 1 | TAA 1 |
| Hv 147 | TCR | TCR | TAA 1 | TAA 2 | TAA 1 | TAA 1 |
| Hv 148 | TAA 2 | TCR | TCR | TAA 1 | TAA 1 | TAA 1 |
| Hv 149 | TCR | TAA 2 | TCR | TAA 1 | TAA 1 | TAA 1 |
| Hv 150 | TCR | TCR | TAA 2 | TAA 1 | TAA 1 | TAA 1 |
| Hv 151 | TAA 1 | TAA 1 | TAA 2 | TAA 2 | TCR | TCR |
| Hv 152 | TAA 1 | TAA 1 | TAA 2 | TCR | TAA 2 | TCR |
| Hv 153 | TAA 1 | TAA 1 | TAA 2 | TCR | TCR | TAA 2 |
| Hv 154 | TAA 1 | TAA 1 | TCR | TAA 2 | TAA 2 | TCR |
| Hv 155 | TAA 1 | TAA 1 | TCR | TAA 2 | TCR | TAA 2 |
| Hv 156 | TAA 1 | TAA 1 | TCR | TCR | TAA 2 | TAA 2 |
| Hv 157 | TAA 1 | TAA 2 | TAA 1 | TAA 2 | TCR | TCR |
| Hv 158 | TAA 1 | TAA 2 | TAA 1 | TCR | TAA 2 | TCR |
| Hv 159 | TAA 1 | TAA 2 | TAA 1 | TCR | TCR | TAA 2 |
| Hv 160 | TAA 1 | TCR | TAA 1 | TAA 2 | TAA 2 | TCR |
| Hv 161 | TAA 1 | TCR | TAA 1 | TAA 2 | TCR | TAA 2 |
| Hv 162 | TAA 1 | TCR | TAA 1 | TCR | TAA 2 | TAA 2 |
| Hv 163 | TAA 1 | TAA 2 | TAA 2 | TAA 1 | TCR | TCR |
| Hv 164 | TAA 1 | TAA 2 | TCR | TAA 1 | TAA 2 | TCR |
| Hv 165 | TAA 1 | TAA 2 | TCR | TAA 1 | TCR | TAA 2 |
| Hv 166 | TAA 1 | TCR | TAA 2 | TAA 1 | TAA 2 | TCR |
| Hv 167 | TAA 1 | TCR | TAA 2 | TAA 1 | TCR | TAA 2 |
| Hv 168 | TAA 1 | TCR | TCR | TAA 1 | TAA 2 | TAA 2 |
| Hv 169 | TAA 1 | TAA 2 | TAA 2 | TCR | TAA 1 | TCR |
| Hv 170 | TAA 1 | TAA 2 | TCR | TAA 2 | TAA 1 | TCR |
| Hv 171 | TAA 1 | TAA 2 | TCR | TCR | TAA 1 | TAA 2 |
| Hv 172 | TAA 1 | TCR | TAA 2 | TAA 2 | TAA 1 | TCR |
| Hv 173 | TAA 1 | TCR | TAA 2 | TCR | TAA 1 | TAA 2 |
| Hv 174 | TAA 1 | TCR | TCR | TAA 2 | TAA 1 | TAA 2 |
| Hv 175 | TAA 1 | TAA 2 | TAA 2 | TCR | TCR | TAA 1 |
| Hv 176 | TAA 1 | TAA 2 | TCR | TAA 2 | TCR | TAA 1 |
| Hv 177 | TAA 1 | TAA 2 | TCR | TCR | TAA 2 | TAA 1 |
| Hv 178 | TAA 1 | TCR | TAA 2 | TAA 2 | TCR | TAA 1 |
| Hv 179 | TAA 1 | TCR | TAA 2 | TCR | TAA 2 | TAA 1 |
| Hv 180 | TAA 1 | TCR | TCR | TAA 2 | TAA 2 | TAA 1 |
| Hv 181 | TAA 2 | TAA 1 | TAA 1 | TAA 2 | TCR | TCR |
| Hv 182 | TAA 2 | TAA 1 | TAA 1 | TCR | TAA 2 | TCR |
| Hv 183 | TAA 2 | TAA 1 | TAA 1 | TCR | TCR | TAA 2 |
| Hv 184 | TCR | TAA 1 | TAA 1 | TAA 2 | TAA 2 | TCR |
| Hv 185 | TCR | TAA 1 | TAA 1 | TAA 2 | TCR | TAA 2 |
| Hv 186 | TCR | TAA 1 | TAA 1 | TCR | TAA 2 | TAA 2 |
| Hv 187 | TAA 2 | TAA 1 | TAA 2 | TAA 1 | TCR | TCR |
| Hv 188 | TAA 2 | TAA 1 | TCR | TAA 1 | TAA 2 | TCR |
| Hv 189 | TAA 2 | TAA 1 | TCR | TAA 1 | TCR | TAA 2 |
| Hv 190 | TCR | TAA 1 | TAA 2 | TAA 1 | TAA 2 | TCR |
| Hv 191 | TCR | TAA 1 | TAA 2 | TAA 1 | TCR | TAA 2 |
| Hv 192 | TCR | TAA 1 | TCR | TAA 1 | TAA 2 | TAA 2 |
| Hv 193 | TAA 2 | TAA 1 | TAA 2 | TCR | TAA 1 | TCR |
| Hv 194 | TAA 2 | TAA 1 | TCR | TAA 2 | TAA 1 | TCR |
| Hv 195 | TAA 2 | TAA 1 | TCR | TCR | TAA 1 | TAA 2 |
| Hv 196 | TCR | TAA 1 | TAA 2 | TAA 2 | TAA 1 | TCR |
| Hv 197 | TCR | TAA 1 | TAA 2 | TCR | TAA 1 | TAA 2 |
| Hv 198 | TCR | TAA 1 | TCR | TAA 2 | TAA 1 | TAA 2 |
| Hv 199 | TAA 2 | TAA 1 | TAA 2 | TCR | TCR | TAA 1 |
| Hv 200 | TAA 2 | TAA 1 | TCR | TAA 2 | TCR | TAA 1 |
| Hv 201 | TAA 2 | TAA 1 | TCR | TCR | TAA 2 | TAA 1 |
| Hv 202 | TCR | TAA 1 | TAA 2 | TAA 2 | TCR | TAA 1 |
| Hv 203 | TCR | TAA 1 | TAA 2 | TCR | TAA 2 | TAA 1 |
| Hv 204 | TCR | TAA 1 | TCR | TAA 2 | TAA 2 | TAA 1 |
| Hv 205 | TAA 2 | TAA 2 | TAA 1 | TAA 1 | TCR | TCR |
| Hv 206 | TAA 2 | TCR | TAA 1 | TAA 1 | TAA 2 | TCR |
| Hv 207 | TAA 2 | TCR | TAA 1 | TAA 1 | TCR | TAA 2 |
| Hv 208 | TCR | TAA 2 | TAA 1 | TAA 1 | TAA 2 | TCR |
| Hv 209 | TCR | TAA 2 | TAA 1 | TAA 1 | TCR | TAA 2 |
| Hv 210 | TCR | TCR | TAA 1 | TAA 1 | TAA 2 | TAA 2 |
| Hv 211 | TAA 2 | TAA 2 | TAA 1 | TCR | TAA 1 | TCR |
| Hv 212 | TAA 2 | TCR | TAA 1 | TAA 2 | TAA 1 | TCR |
| Hv 213 | TAA 2 | TCR | TAA 1 | TCR | TAA 1 | TAA 2 |
| Hv 214 | TCR | TAA 2 | TAA 1 | TAA 2 | TAA 1 | TCR |
| Hv 215 | TCR | TAA 2 | TAA 1 | TCR | TAA 1 | TAA 2 |
| Hv 216 | TCR | TCR | TAA 1 | TAA 2 | TAA 1 | TAA 2 |
| Hv 217 | TAA 2 | TAA 2 | TAA 1 | TCR | TCR | TAA 1 |
| Hv 218 | TAA 2 | TCR | TAA 1 | TAA 2 | TCR | TAA 1 |
| Hv 219 | TAA 2 | TCR | TAA 1 | TCR | TAA 2 | TAA 1 |
| Hv 220 | TCR | TAA 2 | TAA 1 | TAA 2 | TCR | TAA 1 |
| Hv 221 | TCR | TAA 2 | TAA 1 | TCR | TAA 2 | TAA 1 |
| Hv 222 | TCR | TCR | TAA 1 | TAA 2 | TAA 2 | TAA 1 |
| Hv 223 | TAA 2 | TAA 2 | TCR | TAA 1 | TAA 1 | TCR |
| Hv 224 | TAA 2 | TCR | TAA 2 | TAA 1 | TAA 1 | TCR |
| Hv 225 | TAA 2 | TCR | TCR | TAA 1 | TAA 1 | TAA 2 |
| Hv 226 | TCR | TAA 2 | TAA 2 | TAA 1 | TAA 1 | TCR |
| Hv 227 | TCR | TAA 2 | TCR | TAA 1 | TAA 1 | TAA 2 |
| Hv 228 | TCR | TCR | TAA 2 | TAA 1 | TAA 1 | TAA 2 |
| Hv 229 | TAA 2 | TAA 2 | TCR | TAA 1 | TCR | TAA 1 |
| Hv 230 | TAA 2 | TCR | TAA 2 | TAA 1 | TCR | TAA 1 |
| Hv 231 | TAA 2 | TCR | TCR | TAA 1 | TAA 2 | TAA 1 |
| Hv 232 | TCR | TAA 2 | TAA 2 | TAA 1 | TCR | TAA 1 |
| Hv 233 | TCR | TAA 2 | TCR | TAA 1 | TAA 2 | TAA 1 |
| Hv 234 | TCR | TCR | TAA 2 | TAA 1 | TAA 2 | TAA 1 |
| Hv 235 | TAA 2 | TAA 2 | TCR | TCR | TAA 1 | TAA 1 |
| Hv 236 | TAA 2 | TCR | TAA 2 | TCR | TAA 1 | TAA 1 |
| Hv 237 | TAA 2 | TCR | TCR | TAA 2 | TAA 1 | TAA 1 |
| Hv 238 | TCR | TAA 2 | TAA 2 | TCR | TAA 1 | TAA 1 |
| Hv 239 | TCR | TAA 2 | TCR | TAA 2 | TAA 1 | TAA 1 |
| Hv 240 | TCR | TCR | TAA 2 | TAA 2 | TAA 1 | TAA 1 |
| Hv 241 | TAA 1 | TAA 1 | TAA 2 | TCR | TCR | TCR |
| Hv 242 | TAA 1 | TAA 1 | TCR | TAA 2 | TCR | TCR |
| Hv 243 | TAA 1 | TAA 1 | TCR | TCR | TAA 2 | TCR |
| Hv 244 | TAA 1 | TAA 1 | TCR | TCR | TCR | TAA 2 |
| Hv 245 | TAA 1 | TAA 2 | TAA 1 | TCR | TCR | TCR |
| Hv 246 | TAA 1 | TCR | TAA 1 | TAA 2 | TCR | TCR |
| Hv 247 | TAA 1 | TCR | TAA 1 | TCR | TAA 2 | TCR |
| Hv 248 | TAA 1 | TCR | TAA 1 | TCR | TCR | TAA 2 |
| Hv 249 | TAA 1 | TAA 2 | TCR | TAA 1 | TCR | TCR |
| Hv 250 | TAA 1 | TCR | TAA 2 | TAA 1 | TCR | TCR |
| Hv 251 | TAA 1 | TCR | TCR | TAA 1 | TAA 2 | TCR |
| Hv 252 | TAA 1 | TCR | TCR | TAA 1 | TCR | TAA 2 |
| Hv 253 | TAA 1 | TAA 2 | TCR | TCR | TAA 1 | TCR |
| Hv 254 | TAA 1 | TCR | TAA 2 | TCR | TAA 1 | TCR |
| Hv 255 | TAA 1 | TCR | TCR | TAA 2 | TAA 1 | TCR |
| Hv 256 | TAA 1 | TCR | TCR | TCR | TAA 1 | TAA 2 |
| Hv 257 | TAA 1 | TAA 2 | TCR | TCR | TCR | TAA 1 |
| Hv 258 | TAA 1 | TCR | TAA 2 | TCR | TCR | TAA 1 |
| Hv 259 | TAA 1 | TCR | TCR | TAA 2 | TCR | TAA 1 |
| Hv 260 | TAA 1 | TCR | TCR | TCR | TAA 2 | TAA 1 |
| Hv 261 | TAA 2 | TAA 1 | TAA 1 | TCR | TCR | TCR |
| Hv 262 | TCR | TAA 1 | TAA 1 | TAA 2 | TCR | TCR |
| Hv 263 | TCR | TAA 1 | TAA 1 | TCR | TAA 2 | TCR |
| Hv 264 | TCR | TAA 1 | TAA 1 | TCR | TCR | TAA 2 |
| Hv 265 | TAA 2 | TAA 1 | TCR | TAA 1 | TCR | TCR |
| Hv 266 | TCR | TAA 1 | TAA 2 | TAA 1 | TCR | TCR |
| Hv 267 | TCR | TAA 1 | TCR | TAA 1 | TAA 2 | TCR |
| Hv 268 | TCR | TAA 1 | TCR | TAA 1 | TCR | TAA 2 |
| Hv 269 | TAA 2 | TAA 1 | TCR | TCR | TAA 1 | TCR |
| Hv 270 | TCR | TAA 1 | TAA 2 | TCR | TAA 1 | TCR |
| Hv 271 | TCR | TAA 1 | TCR | TAA 2 | TAA 1 | TCR |
| Hv 272 | TCR | TAA 1 | TCR | TCR | TAA 1 | TAA 2 |
| Hv 273 | TAA 2 | TAA 1 | TCR | TCR | TCR | TAA 1 |
| Hv 274 | TCR | TAA 1 | TAA 2 | TCR | TCR | TAA 1 |
| Hv 275 | TCR | TAA 1 | TCR | TAA 2 | TCR | TAA 1 |
| Hv 276 | TCR | TAA 1 | TCR | TCR | TAA 2 | TAA 1 |
| Hv 277 | TAA 2 | TCR | TAA 1 | TAA 1 | TCR | TCR |
| Hv 278 | TCR | TAA 2 | TAA 1 | TAA 1 | TCR | TCR |
| Hv 279 | TCR | TCR | TAA 1 | TAA 1 | TAA 2 | TCR |
| Hv 280 | TCR | TCR | TAA 1 | TAA 1 | TCR | TAA 2 |
| Hv 281 | TAA 2 | TCR | TAA 1 | TCR | TAA 1 | TCR |
| Hv 282 | TCR | TAA 2 | TAA 1 | TCR | TAA 1 | TCR |
| Hv 283 | TCR | TCR | TAA 1 | TAA 2 | TAA 1 | TCR |
| Hv 284 | TCR | TCR | TAA 1 | TCR | TAA 1 | TAA 2 |
| Hv 285 | TAA 2 | TCR | TAA 1 | TCR | TCR | TAA 1 |
| Hv 286 | TCR | TAA 2 | TAA 1 | TCR | TCR | TAA 1 |
| Hv 287 | TCR | TCR | TAA 1 | TAA 2 | TCR | TAA 1 |
| Hv 288 | TCR | TCR | TAA 1 | TCR | TAA 2 | TAA 1 |
| Hv 289 | TAA 2 | TCR | TCR | TAA 1 | TAA 1 | TCR |
| Hv 290 | TCR | TAA 2 | TCR | TAA 1 | TAA 1 | TCR |
| Hv 291 | TCR | TCR | TAA 2 | TAA 1 | TAA 1 | TCR |
| Hv 292 | TCR | TCR | TCR | TAA 1 | TAA 1 | TAA 2 |
| Hv 293 | TAA 2 | TCR | TCR | TAA 1 | TCR | TAA 1 |
| Hv 294 | TCR | TAA 2 | TCR | TAA 1 | TCR | TAA 1 |
| Hv 295 | TCR | TCR | TAA 2 | TAA 1 | TCR | TAA 1 |
| Hv 296 | TCR | TCR | TCR | TAA 1 | TAA 2 | TAA 1 |
| Hv 297 | TAA 2 | TCR | TCR | TCR | TAA 1 | TAA 1 |
| Hv 298 | TCR | TAA 2 | TCR | TCR | TAA 1 | TAA 1 |
| Hv 299 | TCR | TCR | TAA 2 | TCR | TAA 1 | TAA 1 |
| Hv 300 | TCR | TCR | TCR | TAA 2 | TAA 1 | TAA 1 |
| Hv 301 | TAA 1 | TAA 2 | TCR | TCR | TCR | TCR |
| Hv 302 | TAA 1 | TCR | TAA 2 | TCR | TCR | TCR |
| Hv 303 | TAA 1 | TCR | TCR | TAA 2 | TCR | TCR |
| Hv 304 | TAA 1 | TCR | TCR | TCR | TAA 2 | TCR |
| Hv 305 | TAA 1 | TCR | TCR | TCR | TCR | TAA 2 |
| Hv 306 | TAA 2 | TAA 1 | TCR | TCR | TCR | TCR |
| Hv 307 | TCR | TAA 1 | TAA 2 | TCR | TCR | TCR |
| Hv 308 | TCR | TAA 1 | TCR | TAA 2 | TCR | TCR |
| Hv 309 | TCR | TAA 1 | TCR | TCR | TAA 2 | TCR |
| Hv 310 | TCR | TAA 1 | TCR | TCR | TCR | TAA 2 |
| Hv 311 | TAA 2 | TCR | TAA 1 | TCR | TCR | TCR |
| Hv 312 | TCR | TAA 2 | TAA 1 | TCR | TCR | TCR |
| Hv 313 | TCR | TCR | TAA 1 | TAA 2 | TCR | TCR |
| Hv 314 | TCR | TCR | TAA 1 | TCR | TAA 2 | TCR |
| Hv 315 | TCR | TCR | TAA 1 | TCR | TCR | TAA 2 |
| Hv 316 | TAA 2 | TCR | TCR | TAA 1 | TCR | TCR |
| Hv 317 | TCR | TAA 2 | TCR | TAA 1 | TCR | TCR |
| Hv 318 | TCR | TCR | TAA 2 | TAA 1 | TCR | TCR |
| Hv 319 | TCR | TCR | TCR | TAA 1 | TAA 2 | TCR |
| Hv 320 | TCR | TCR | TCR | TAA 1 | TCR | TAA 2 |
| Hv 321 | TAA 2 | TCR | TCR | TCR | TAA 1 | TCR |
| Hv 322 | TCR | TAA 2 | TCR | TCR | TAA 1 | TCR |
| Hv 323 | TCR | TCR | TAA 2 | TCR | TAA 1 | TCR |
| Hv 324 | TCR | TCR | TCR | TAA 2 | TAA 1 | TCR |
| Hv 325 | TCR | TCR | TCR | TCR | TAA 1 | TAA 2 |
| Hv 326 | TAA 2 | TCR | TCR | TCR | TCR | TAA 1 |
| Hv 327 | TCR | TAA 2 | TCR | TCR | TCR | TAA 1 |
| Hv 328 | TCR | TCR | TAA 2 | TCR | TCR | TAA 1 |
| Hv 329 | TCR | TCR | TCR | TAA 2 | TCR | TAA 1 |
| Hv 330 | TCR | TCR | TCR | TCR | TAA 2 | TAA 1 |

### 7.5. TCR ABMs

The MBMs (e.g., TBMs) of the disclosure contain an ABM that specifically binds to a component of a TCR complex. The TCR is a disulfide-linked membrane-anchored heterodimeric protein normally consisting of the highly variable alpha (α) and beta (β) chains expressed as part of a complex with the invariant CD3 chain molecules. T cells expressing this receptor are referred to as α:β (or αβ) T cells, though a minority of T cells express an alternate receptor, formed by variable gamma (γ) and delta (δ) chains, referred as γδ T cells.

In an embodiment, MBMs of the disclosure contain an ABM that specifically binds to CD3.

### 7.5.1. CD3 ABMs

The MBMs (e.g., TBMs) of the disclosure can contain an ABM that specifically binds to CD3. The term “CD3” refers to the cluster of differentiation 3 co-receptor (or co-receptor complex, or polypeptide chain of the co-receptor complex) of the T cell receptor. The amino acid sequence of the polypeptide chains of human CD3 are provided in NCBI Accession P04234, P07766 and P09693. CD3 proteins may also include variants. CD3 proteins may also include fragments. CD3 proteins also include post-translational modifications of the CD3 amino acid sequences. Post-translational modifications include, but are not limited to, N- and O-linked glycosylation.

In some embodiments, a MBM (e.g., TBM) of the disclosure can comprise an ABM which is an anti-CD3 antibody (e.g., as described in US 2016/0355600, WO 2014/110601, and WO 2014/145806) or an antigen-binding domain thereof. Exemplary anti-CD3 VH, VL, and scFV sequences that can be used in MBMs (e.g., TBMs) of the disclosure are provided in Table 8A.

TABLE 8A
CD3 Binders-Variable domain sequences

| | | | SEQ |
|---|---|---|---|
| Binding | | | ID |
| Domain | Chain | Sequence | NO: |
| CD3-1 | VH | QVQLQQSGAELARPGASVKMSCKASGYTFTRYTMHWVKQRPGQG | 78 |
| | | LEWIGYINPSRGYTNYNQKFKDKATLTTDKSSSTAYMQLSSLTS | |
| | | EDSAVYYCARYYDDHYCLDYWGQGTTLTVSS | |
| | VL | QIVLTQSPAIMSASPGEKVTMTCSASSSVSYMNWYQQKSGTSPK | 79 |
| | | RWIYDTSKLASGVPAHFRGSGSGTSYSLTISGMEAEDAATYYCQ | |
| | | QWSSNPFTFGSGTKLEIN | |
| CD3-2 | VH | EVQLVESGGGLVQPKGSLKLSCAASGFTFNTYAMNWVRQAPGKG | 80 |
| | | LEWVARIRSKYNNYATYYADSVKDRFTISRDDSQSILYLQMNNL | |
| | | KTEDTAMYYCVRHGNFGNSYVSWFAYWGQGTLVTVSA | |
| | VL | QAWTQESALTTSPGETVTLTCRSSTGAVTTSNYANWVQEKPDHL | 81 |
| | | FTGLIGGTNKRAPGVPARFSGSLIGDKAALTITGAQTEDEAIYF | |
| | | CALWYSNLWVFGGGTKLTVL | |
| CD3-3 | VH | QVQLQQSGAELARPGASVKMSCKASGYTFTSYTMHWVKQRPGQG | 82 |
| | | LEWIGYINPSSGYTKYNQKFKDKATLTADKSSSTAYMQLSSLTS | |
| | | EDSAVYYCARWQDYDVYFDYWGQGTTLTVSS | |
| | VL | QIVLSQSPAILSASPGEKVTMTCRASSSVSYMHWYQQKPGSSPK | 83 |
| | | PWIYATSNLASGVPARFSGSGSGTSYSLTISRVEAEDAATYYCQ | |
| | | QWSSNPPTFGGGTKLETK | |
| CD3-4 | VH | QVQLQQSGAELARPGASVKMSCKASGYTFTRYTMHWVKQRPGQG | 78 |
| | | LEWIGYINPSRGYTNYNQKFKDKATLTTDKSSSTAYMQLSSLTS | |
| | | EDSAVYYCARYYDDHYCLDYWGQGTTLTVSS | |
| | VL | QIVLTQSPAIMSASPGEKVTMTCRASSSVSYMNWYQQKSGTSPK | 84 |
| | | RWIYDTSKVASGVPYRFSGSGSGTSYSLTISSMEAEDAATYYCQ | |
| | | QWSSNPLTFGSGTKLEIN | |
| CD3-5 | VH | QVQLVQSGGGVVQPGRSLRLSCKASGYTFTRYTMHWVRQAPGKG | 85 |
| | | LEWIGYINPSRGYTNYNQKVKDRFTISRDNSKNTAFLQMDSLRP | |
| | | EDTGVYFCARYYDDHYCLDYWGQGTPVTVSS | |
| | VL | DIQMTQSPSSLSASVGDRVTITCSASSSVSYMNWYQQTPGKAPK | 86 |
| | | RWIYDTSKLASGVPSRFSGSGSGTDYTFTISSLQPEDIATYYCQ | |
| | | QWSSNPFTFGQGTKLQIT | |
| CD3-6 | VH | QVQLVESGGGVVQPGRSLRLSCAASGFKFSGYGMHWVRQAPGKG | 87 |
| | | LEWVAVIWYDGSKKYYVDSVKGRFTISRDNSKNTLYLQMNSLRA | |
| | | EDTAVYYCARQMGYWHFDLWGRGTLVTVSS | |
| | VL | EIVLTQSPATLSLSPGERATLSCRASQSVSSYLAWYQQKPGQAP | 88 |
| | | RLLIYDASNRATGIPARFSGSGSGTDFTLTISSLEPEDFAVYYC | |
| | | QQRSNWPPLTFGGGTKVEIK | |
| CD3-7 | VH | EVQLVESGGGLVQPGGSLRLSCAASGFTFSTYAMNWVRQAPGKG | 89 |
| | | LEWVGRIRSKYNNYATYYADSVKDRFISRDDSKNSLYLQMNSLK | |
| | | TEDTAVYYCVRHGNFGNSYVSWFAYWGQGTLVTVSS | |
| | VL | QAWTQEPSLTVSPGGTVTLTCRSSTGAVTTSNYANWVQQKPGQA | 90 |
| | | PRGLIGGTNKRAPWTPARFSGSLLGGKAALIGAQAEDEADYYCA | |
| | | LWYSNLWVFGGGTKLTVL | |
| CD3-8 | VH | DIKLQQSGAELARPGASVKMSCKTSGYTFTRYTMHWVKQRPGQG | 91 |
| | | LEWIGYINPSRGYTNYNQKFKDKATLTTDKSSSTAYMQLSSLTS | |
| | | EDSAVYYCARYYDDHYCLDYWGQGTTLTVSS | |
| | VL | DIQLTQSPAIMSASPGEKVTMTCRASSSVSYMNWYQQKSGTSPK | 92 |
| | | RWIYDTSKVASGVPYRFSGSGSGTSYSLISSMEAEDAATYYCQQ | |
| | | WSSNPLTFGAGTKLELK | |
| CD3-9 | VH | EVQLVESGGGLVQPGGSLRLSCAASGFTFNTYAMNWVRQAPGKG | 93 |
| | | LEWVARIRSKYNNYATYYADSVKDRFISRDDSKNSLYLQMNSLK | |
| | | TEDTAVYYCVRHGNFGNSYVSWFAYWGQGTLVTVSS | |
| | VL | QAWTQEPSLTVSPGGTVTLTCRSSTGAVTTSNYANWVQQKPGQA | 90 |
| | | PRGLIGGTNKRAPWTPARFSGSLLGGKAALIGAQAEDEADYYCA | |
| | | LWYSNLWVFGGGTKLTVL | |
| CD3-10 | VH | EVKLLESGGGLVQPKGSLKLSCAASGFTFNTYAMNWVRQAPGKG | 94 |
| | | LEWVARIRSKYNNYATYYADSVKDRFTISRDDSQSILYLQMNNL | |
| | | KTEDTAMYYCVRHGNFGNSYVSWFAYWGQGTLVTVSA | |
| | VL | QAWTQESALTTSPGETVTLTCRSSTGAVTTSNYANWVQEKPDHL | 81 |
| | | FTGLIGGTNKRAPGVPARFSGSLIGDKAALTITGAQTEDEAIYF | |
| | | CALWYSNLWVFGGGTKLTVL | |
| CD3-11 | VH | EVQLVESGGGLVQPGGSLKLSCAASGFTFNSYAMNWVRQAPGKG | 95 |
| | | LEWVARIRSKYNNYATYYADSVKGRFTISRDDSKNTAYLQMNNL | |
| | | KTEDTAVYYCVRHGNFGNSYVSWWAYWGQGTLVTVSS | |
| | VL | QTVVTQEPSLTVSPGGTVTLTCGSSTGAVTSGNYPNWVQQKPGQ | 96 |
| | | APRGLIGGTKFLAPGTPQRFSGSLLGGKAALTLSGVQPEDEAEY | |
| | | YCVLWYSNRWVFGGGTKLTVL | |
| CD3-12 | VH | EVQLVESGGGLVQPGGSLKLSCAASGFTFNKYAMNWVRQAPGKG | 97 |
| | | LEWVARIRSKYNNYATYYADSVKDRFTISRDDSKNTAYLQMNNL | |
| | | KTEDTAVYYCVRHGNFGNSYISYWAYWGQGTLVTVSS | |
| | VL | QTVVTQEPSLTVSPGGTVTLTCGSSTGAVTSGNYPNWVQQKPGQ | 98 |
| | | APRGLIGGTKFLAPGTPARFSGSLLGGKAALTLSGVQPEDEAEY | |
| | | YCVLWYSNRWVFGGGTKLTVL | |
| CD3-13 | VH | QVQLVQSGAEVKKPGASVKVSCKASGYTFTRYTMHWVRQAPGQGL | 99 |
| | | EWMGYINPSRGYTNYNQKFKDRVTMTTDTSISTAYMELSRLRSDD | |
| | | TAVYYCARYYDDHYCLDYWGQGTLVTVSS | |
| | VL | EIVLTQSPATLSLSPGERATLSCSASSSVSYMNWYQQKPGQAPRL | 100 |
| | | LIYDTSKLASGVPAHFRGSGSGTDFTLTISSLEPEDFAVYYCQQW | |
| | | SSNPFTFGQGTKVEIK | |
| CD3-14 | VH | EVQLVESGGGLVQPGGSLRLSCAASGFTFSTYAMNWVRQAPGKGL | 101 |
| | | EWVSRIRSKYNNYATYYADSVKDRFTISRDDSKNTLYLQMNSLRA | |
| | | EDTAVYYCARHGNFGNSYVSWFAYWGQGTMVTVSS | |
| | VL | QAVVTQEPSLTVSPGGTVTLTCRSSTGAVTTSNYANWVQQKPGQA | 102 |
| | | PRGLIGGTNKRAPGTPARFSGSLLGGKAALTLSGVQPEDEAEYYC | |
| | | ALWYSNLWVFGGGTKLTVL | |
| CD3-15 | VH | EVQLVESGGGLVQPGGSLRLSCAASGFTFNTYAMNWVRQAPGKGL | 103 |
| | | EWVGRIRSKYNNYATYYADSVKDRFTISRDDSKNSLYLQMNSLKT | |
| | | EDTAVYYCVRHGNFGNSYVSWFAYWGQGTLVTVSS | |
| | VL | QAVVTQEPSLTVSPGGTVTLTCRSSTGAVTTSNYANWVQEKPGQA | 104 |
| | | PRGLIGGTNKRAPWTPARFSGSLLGGKAALTITGAQAEDEADYYC | |
| | | ALWYSNLWVFGGGTKLTVL | |
| CD3-16 | VH | EVQLVESGGGLVQPGGSLRLSCAASGFTFNTYAMNWVRQAPGKGL | 105 |
| | | EWVGRIRSKYNNYATYYADSVKGRFTISRDDSKNTLYLQMNSLRA | |
| | | EDTAVYYCVRHGNFGNSYVSWFAYWGQGTLVTVSS | |
| | VL | QAVVTQEPSLTVSPGGTVTLTCGSSTGAVTTSNYANWVQQKPGQA | 106 |
| | | PRGLIGGTNKRAPGVPARFSGSLLGGKAALTLSGAQPEDEAEYYC | |
| | | ALWYSNLWVFGGGTKLTVL | |
| CD3-17 | VH | EVQLVESGGGLVQPGGSLRLSCAASGFTFSTYAMNWVRQAPGKGL | 107 |
| | | EWVGRIRSKYNNYATYYADSVKGRFTISRDDSKNTLYLQMNSLRA | |
| | | EDTAVYYCVRHGNFGDSYVSWFAYWGQGTLVTVSS | |
| | VL | QAVVTQEPSLTVSPGGTVTLTCGSSTGAVTTSNYANWVQQKPGKS | 108 |
| | | PRGLIGGTNKRAPGVPARFSGSLLGGKAALTISGAQPEDEADYYC | |
| | | ALWYSNHWVFGGGTKLTVL | |
| CD3-18 | VH | QVQLVQSGGGVVQPGRSLRLSCKASGYTFTRYTMHWVRQAPGKGL | 85 |
| | | EWIGYINPSRGYTNYNQKVKDRFTISRDNSKNTAFLQMDSLRPED | |
| | | TGVYFCARYYDDHYCLDYWGQGTPVTVSS | |
| | VL | DIQMTQSPSSLSASVGDRVTITCSASSSVSYMNWYQQTPGKAPKR | 109 |
| | | WIYDTSKLASGVPSRFSGSGSGTDYTFTISSLQPEDIATYYCQQW | |
| | | SSNPFTFGQGT | |
| CD3-19 | VH | QVQLVQSGGGVVQPGRSLRLSCKASGYTFTRYTMHWVRQAPGKG | 110 |
| | | LEWIGYINPSRGYTNYNQKVKDRFTISRDNSKNTAFLQMDSLRPE | |
| | | DTGVYFCARYYDDHYSLDYWGQGTPVTVSS | |
| | VL | DIQMTQSPSSLSASVGDRVTITCSASSSVSYMNWYQQTPGKAPKR | 109 |
| | | WIYDTSKLASGVPSRFSGSGSGTDYTFTISSLQPEDIATYYCQQW | |
| | | SSNPFTFGQGT | |
| CD3-20 | VH | EVQLQQSGPELVKPGASMKISCKASGYSFTGYTMNWVKQSHGKNL | 111 |
| | | EWMGLINPYKGVSTYNQKFKDKATLTVDKSSSTAYMELLSLTSED | |
| | | SAVYYCARSGYYGDSDWYFDVWGQGTTLTVFS | |
| | VL | DIQMTQTTSSLSASLGDRVTISCRASQDIRNYLNWYQQKPDGTVK | 112 |
| | | LLIYYTSRLHSGVPSKFSGSGSGTDYSLTISNLEQEDIATYFCQQ | |
| | | GNTLPWTFAGGTKLEIK | |
| CD3-21 | VH | EVQLVESGGGLVQPGGSLKLSCAASGFTFNTYAMNWVRQASGKGL | 113 |
| | | EWVGRIRSKYNNYATYYADSVKDRFTISRDDSKSTLYLQMNSLKT | |
| | | EDTAVYYCVRHGNFGNSYVSWFAYWGQGTLVTVSS | |
| | VL | QAWTQEPSLTVSPGGTVTLTCRSSTGAVTTSNYANWVQQKPGQA | 114 |
| | | PRGLIGGTNKRAPWTPARFSGSLLGDKAALTLSGAQPEDEAEYF | |
| | | CALWYSNLVWFGGGTKLTVL | |
| | scFv | EVQLVESGGGLVQPGGSLKLSCAASGFTFNTYAMNWVRQASGKGL | 115 |
| | | EWVGRIRSKYNNYATYYADSVKDRFTISRDDSKSTLYLQMNSLKT | |
| | | EDTAVYYCVRHGNFGNSYVSWFAYWGQGTLVTVSSGGGGSGGGGS | |
| | | GGGGSGGGGSQAWTQEPSLTVSPGGTVTLTCRSSTGAVTTSNYAN | |
| | | WVQQKPGQAPRGLIGGTNKRAPWTPARFSGSLLGDKAALTLSGAQ | |
| | | PEDEAEYFCALWYSNLWVFGGGTKLTVL | |
| CD3-22 | VH | EVQLVESGGGLVQPGGSLRLSCAASGFTFSTYAMNWVRQAPGKGL | 107 |
| | | EWVGRIRSKYNNYATYYADSVKGRFTISRDDSKNTLYLQMNSLRAE | |
| | | DTAVYYCVRHGNFGDSYVSWFAYWGQGTLVTVSS | |
| | VL | QAWTQEPSLTVSPGGTVTLTCGSSTGAVTTSNYANWVQQKPGKSP | 108 |
| | | RGLIGGTNKRAPGVPARFSGSLLGGKAALTISGAQPEDEADYYCA | |
| | | LWYSNHWVFGGGTKLTVL | |
| | scFv | EVQLVESGGGLVQPGGSLRLSCAASGFTFSTYAMNWVRQAPGKGL | 116 |
| | | EWVGRIRSKYNNYATYYADSVKGRFTISRDDSKNTLYLQMNSLRA | |
| | | EDTAVYYCVRHGNFGDSYVSWFAYWGQGTLVTVSSGKPGSGKPGS | |
| | | GKPGSGKPGSQAWTQEPSLTVSPGGTVTLTCGSSTGAVTTSNYAN | |
| | | WVQQKPGKSPRGLIGGTNKRAPGVPARFSGSLLGGKAALTISGAQ | |
| | | PEDEADYYCALWYSNHWVFGGGTKLTVL | |
| CD3-23 | VH | EVQLVESGGGLVQPGGSLRLSCAASGFTFSTYAMNWVRQAPGKGL | 117 |
| | | EWVGRIRSKANNYATYYADSVKGRFTISRDDSKNTLYLQMNSLRA | |
| | | EDTAVYYCVRHGNFGDSYVSWFAYWGQGTLVTVSS | |
| | VL | QAWTQEPSLTVSPGGTVTLTCGSSTGAVTTSNYANWVQQKPGKS | 108 |
| | | PRGLIGGTNKRAPGVPARFSGSLLGGKAALTISGAQPEDEADYYC | |
| | | ALWYSNHWVFGGGTKLTVL | |
| | scFv | EVQLVESGGGLVQPGGSLRLSCAASGFTFSTYAMNWVRQAPGKGL | 118 |
| | | EWVGRIRSKANNYATYYADSVKGRFTISRDDSKNTLYLQMNSLRA | |
| | | EDTAVYYCVRHGNFGDSYVSWFAYWGQGTLVTVSSGKPGSGKPGS | |
| | | GKPGSGKPGSQAVVTQEPSLTVSPGGTVTLTCGSSTGAVTTSNYA | |
| | | NWVQQKPGKSPRGLIGGTNKRAPGVPARFSGSLLGGKAALTISGA | |
| | | QPEDEADYYCALWYSNHWVFGGGTKLTVL | |
| CD3-24 | VH | EVQLVESGGGLVQPGGSLRLSCAASGFTFSTYAMNWVRQAPGKGL | 119 |
| | | EWVGRIRSKYNNYATYYADSVKGRFTISRDDSKNTLYLQMNSLRA | |
| | | EDTAVYYCVRHGNFGDEYVSWFAYWGQGTLVTVSS | |
| | VL | QAWTQEPSLTVSPGGTVTLTCGSSTGAVTTSNYANWVQQKPGKSP | 108 |
| | | RGLIGGTNKRAPGVPARFSGSLLGGKAALTISGAQPEDEADYYCA | |
| | | LWYSNHWVFGGGTKLTVL | |
| | scFv | EVQLVESGGGLVQPGGSLRLSCAASGFTFSTYAMNWVRQAPGKGL | 120 |
| | | EWVGRIRSKYNNYATYYADSVKGRFTISRDDSKNTLYLQMNSLRA | |
| | | EDTAVYYCVRHGNFGDEYVSWFAYWGQGTLVTVSSGKPGSGKPGS | |
| | | GKPGSGKPGSQAVVTQEPSLTVSPGGTVTLTCGSSTGAVTTSNYA | |
| | | NWVQQKPGKSPRGLIGGTNKRAPGVPARFSGSLLGGKAALTISGA | |
| | | QPEDEADYYCALWYSNHWVFGGGTKLTVL | |
| CD3-25 | VH | EVQLVESGGGLVQPGGSLRLSCAASGFTFSTYAMNWVRQAPGKGL | 121 |
| | | EWVGRIRSKYNNYATYYADSVKGRFTISRDDSKNTLYLQMNSLRA | |
| | | EDTAVYYCVRHGNFGDPYVSWFAYWGQGTLVTVSS | |
| | VL | QAVVTQEPSLTVSPGGTVTLTCGSSTGAVTTSNYANWVQQKPGKS | 108 |
| | | PRGLIGGTNKRAPGVPARFSGSLLGGKAALTISGAQPEDEADYYC | |
| | | ALWYSNHWVFGGGTKLTVL | |
| | scFv | EVQLVESGGGLVQPGGSLRLSCAASGFTFSTYAMNWVRQAPGKGL | 122 |
| | | EWVGRIRSKYNNYATYYADSVKGRFTISRDDSKNTLYLQMNSLRA | |
| | | EDTAVYYCVRHGNFGDPYVSWFAYWGQGTLVTVSSGKPGSGKPGS | |
| | | GKPGSGKPGSQAVVTQEPSLTVSPGGTVTLTCGSSTGAVTTSNYA | |
| | | NWVQQKPGKSPRGLIGGTNKRAPGVPARFSGSLLGGKAALTISGA | |
| | | QPEDEADYYCALWYSNHWVFGGGTKLTVL | |
| CD3-26 | VH | EVQLVESGGGLVQPGGSLRLSCAASGFTFSTYAMNWVRQAPGKGL | 123 |
| | | EWVGRIRSKYNNYATYYADSVKGRFTISRDDSKNTLYLQMNSLRA | |
| | | EDTAVYYCVRHGNFGDSYVSWFDYWGQGTLVTVSS | |
| | VL | QAWTQEPSLTVSPGGTVTLTCGSSTGAVTTSNYANWVQQKPGKS | 108 |
| | | PRGLIGGTNKRAPGVPARFSGSLLGGKAALTISGAQPEDEADYYC | |
| | | ALWYSNHWVFGGGTKLTVL | |
| | scFv | EVQLVESGGGLVQPGGSLRLSCAASGFTFSTYAMNWVRQAPGKGL | 124 |
| | | EWVGRIRSKYNNYATYYADSVKGRFTISRDDSKNTLYLQMNSLRA | |
| | | EDTAVYYCVRHGNFGDSYVSWFDYWGQGTLVTVSSGKPGSGKPGS | |
| | | GKPGSGKPGSQAVVTQEPSLTVSPGGTVTLTCGSSTGAVTTSNYA | |
| | | NWVQQKPGKSPRGLIGGTNKRAPGVPARFSGSLLGGKAALTISGA | |
| | | QPEDEADYYCALWYSNHWVFGGGTKLTVL | |
| CD3-27 | VH | EVQLVESGGGLVQPGGSLRLSCAASGFTFSTYAMSWVRQAPGKGL | 125 |
| | | EWVGRIRSKYNNYATYYADSVKGRFTiSRDDSKNTLYLQMNSLRA | |
| | | EDTAVYYCVRHGNFGDSYVSWFAYWGQGTLVTVSS | |
| | VL | QAVVTQEPSLTVSPGGTVTLTCGSSTGAVTTSNYANWVQQKPGK | 108 |
| | | SPRGLIGGTNKRAPGVPARFSGSLLGGKAALTISGAQPEDEADY | |
| | | YCALWYSNHWVFGGGTKLTVL | |
| | scFv | EVQLVESGGGLVQPGGSLRLSCAASGFTFSTYAMSWVRQAPGKG | 126 |
| | | LEWVGRIRSKYNNYATYYADSVKGRFTISRDDSKNTLYLQMNSL | |
| | | RAEDTAVYYCVRHGNFGDSYVSWFAYWGQGTLVTVSSGKPGSGK | |
| | | PGSGKPGSGKPGSQAVVTQEPSLTVSPGGTVTLTCGSSTGAVTT | |
| | | SNYANWVQQKPGKSPRGLIGGTNKRAPGVPARFSGSLLGGKAAL | |
| | | TISGAQPEDEADYYCALWYSNHWVFGGGTKLTVL | |
| CD3-28 | VH | EVQLVESGGGLVQPGGSLRLSCAASGFTFNTYAMNWVRQAPGKG | 105 |
| | | LEWVGRIRSKYNNYATYYADSVKGRFTISRDDSKNTLYLQMNSL | |
| | | RAEDTAVYYCVRHGNFGNSYVSWFAYWGQGTLVTVSS | |
| | VL | QAVVTQEPSLTVSPGGTVTLTCGSSTGAVTTSNYANWVQQKPGQ | 106 |
| | | APRGLIGGTNKRAPGVPARFSGSLLGGKAALTLSGAQPEDEAEY | |
| | | YCALWYSNLWVFGGGTKLTVL | |
| | scFv | EVQLVESGGGLVQPGGSLRLSCAASGFTFNTYAMNWVRQAPGKG | 127 |
| | | LEWVGRIRSKYNNYATYYADSVKGRFTISRDDSKNTLYLQMNSL | |
| | | RAEDTAVYYCVRHGNFGNSYVSWFAYWGQGTLVTVSSGGGGSGG | |
| | | GGSGGGGSQAWTQEPSLTVSPGGTVTLTCGSSTGAVTTSNYANW | |
| | | VQQKPGQAPRGLIGGTNKRAPGVPARFSGSLLGGKAALTLSGAQ | |
| | | PEDEAEYYCALWYSNLWVFGGGTKLTVLGSHHHHHH | |

CDR sequences for a number of CD3 binders as defined by the Kabat numbering scheme (Kabat et al, 1991, Sequences of Proteins of Immunological Interest, 5^{th} Ed. Public Health Service, National Institutes of Health, Bethesda, Md.), Chothia numbering scheme (Al-Lazikani et al., 1997, J. Mol. Biol 273:927-948), and a combination of Kabat and Chothia numbering are provided in Tables 8B-8D, respectively.

TABLE 8B
CD3 Binders-CDR sequences according to
Kabat numbering scheme

| | | | SEQ | | SEQ | | SEQ |
|---|---|---|---|---|---|---|---|
| Binding | | | ID | | ID | | ID |
| Domain | Chain | CDR1 | NO: | CDR2 | NO: | CDR3 | NO: |
| CD3-1 | VH | RYTMH | 128 | YINPSRG | 148 | YYDDH | 172 |
| | | | | YTNYNQK | | YCLDY | |
| | | | | FKD | | | |
| | VL | SASSSV | 129 | DTSKLAS | 149 | QQWSS | 173 |
| | | SYMN | | | | NPFT | |
| CD3-2 | VH | TYAMN | 130 | RIRSKYN | 150 | HGNFG | 174 |
| | | | | NYATYYA | | NSYVS | |
| | | | | DSVKD | | WFAY | |
| | VL | RSSTGA | 131 | GTNKRAP | 151 | ALWYS | 175 |
| | | VTTSNY | | | | NLWV | |
| | | AN | | | | | |
| CD3-3 | VH | SYTMH | 132 | YINPSSG | 152 | WQDYD | 176 |
| | | | | YTKYNQK | | VYFDY | |
| | | | | FKD | | | |
| | VL | RASSS | 133 | ATSNLAS | 153 | QQWSS | 177 |
| | | VSYM | | | | NPPT | |
| | | H | | | | | |
| CD3-4 | VH | RYTMH | 128 | YINPSRG | 148 | YYDDH | 172 |
| | | | | YTNYNQK | | YCLDY | |
| | | | | FKD | | | |
| | VL | RASSS | 134 | DTSKVAS | 154 | QQWSS | 178 |
| | | VSYM | | | | NPLT | |
| | | N | | | | | |
| CD3-5 | VH | RYTMH | 128 | YINPSRG | 155 | YYDDH | 172 |
| | | | | YTNYNQK | | YCLDY | |
| | | | | VKD | | | |
| | VL | SASSS | 129 | DTSKLAS | 149 | QQWSS | 173 |
| | | VSYM | | | | NPFT | |
| | | N | | | | | |
| CD3-6 | VH | GYGMH | 135 | VIWYDGS | 156 | QMGYW | 179 |
| | | | | KKYYVDS | | HFDL | |
| | | | | VKG | | | |
| | VL | RASQS | 136 | DASNRAT | 157 | QQRSN | 180 |
| | | VSSY | | | | WPPLT | |
| | | LA | | | | | |
| CD3-7 | VH | TYAMN | 130 | RIRSKYN | 158 | VRHGN | 181 |
| | | | | NYATYYA | | FGNSYV | |
| | | | | D | | SWFAY | |
| | VL | RSSTG | 131 | GTNKRAP | 151 | ALWYS | 175 |
| | | AVTT | | | | NLWV | |
| | | SNYAN | | | | | |
| CD3-8 | VH | RYTMH | 128 | YINPSRGY | 148 | YYDDH | 172 |
| | | | | TNYNQK | | YCLDY | |
| | | | | FKD | | | |
| | VL | RASSS | 134 | DTSKVAS | 154 | QQWSS | 178 |
| | | VSYM | | | | NPLT | |
| | | N | | | | | |
| CD3-9 | VH | TYAMN | 130 | RIRSKYNN | 158 | VRHGNF | 181 |
| | | | | YATYYA | | GNSYV | |
| | | | | D | | SWFAY | |
| | VL | RSSTG | 131 | GTNKRAP | 151 | ALWYS | 175 |
| | | AVTT | | | | NLWV | |
| | | SNYAN | | | | | |
| CD3-10 | VH | TYAMN | 130 | RIRSKYNN | 150 | HGNFGN | 174 |
| | | | | YATYYA | | SYVSW | |
| | | | | DSVKD | | FAY | |
| | VL | RSSTG | 131 | GTNKRAP | 151 | ALWYS | 175 |
| | | AVTT | | | | NLWV | |
| | | SNYAN | | | | | |
| CD3-11 | VH | SYAMN | 137 | RIRSKYNN | 159 | HGNFGN | 182 |
| | | | | YATYYA | | SYVSW | |
| | | | | DSVKG | | WAY | |
| | VL | GSSTG | 138 | GTKFLAP | 160 | VLWYS | 183 |
| | | AVTS | | | | NRWV | |
| | | GNYPN | | | | | |
| CD3-12 | VH | KYAMN | 139 | RIRSKYN | 150 | HGNFGN | 184 |
| | | | | NYATYYA | | SYISY | |
| | | | | DSVKD | | WAY | |
| | VL | GSSTG | 138 | GTKFLAP | 160 | VLWYS | 183 |
| | | AVTS | | | | NRWV | |
| | | GNYPN | | | | | |
| CD3-13 | VH | RYTMH | 128 | YINPSRG | 148 | YYDDH | 172 |
| | | | | YTNYNQK | | YCLDY | |
| | | | | FKD | | | |
| | VL | SASSS | 129 | DTSKLAS | 149 | QQWSS | 173 |
| | | VSYM | | | | NPFT | |
| | | N | | | | | |
| CD3-14 | VH | TYAMN | 130 | RIRSKYN | 150 | HGNFGN | 174 |
| | | | | NYATYYA | | SYVSW | |
| | | | | DSVKD | | FAY | |
| | VL | RSSTG | 131 | GTNKRAP | 151 | ALWY | 175 |
| | | AVTT | | | | SNLWV | |
| | | SNYAN | | | | | |
| CD3-15 | VH | TYAMN | 130 | RIRSKYN | 150 | HGNFGN | 174 |
| | | | | NYATYYA | | SYVSW | |
| | | | | DSVKD | | FAY | |
| | VL | RSSTG | 131 | GTNKRAP | 151 | ALWYS | 175 |
| | | AVTT | | | | NLWV | |
| | | SNYAN | | | | | |
| CD3-16 | VH | TYAMN | 130 | RIRSKYN | 159 | HGNFGN | 174 |
| | | | | NYATYYA | | SYVSW | |
| | | | | DSVKG | | FAY | |
| | VL | GSSTG | 140 | GTNKRAP | 151 | ALWYS | 175 |
| | | AVTT | | | | NLWV | |
| | | SNYAN | | | | | |
| CD3-17 | VH | TYAMN | 130 | RIRSKYN | 159 | HGNFGD | 185 |
| | | | | NYATYYA | | SYVSW | |
| | | | | DSVKG | | FAY | |
| | VL | GSSTG | 140 | GTNKRAP | 151 | ALWYS | 186 |
| | | AVTT | | | | NHWV | |
| | | SNYAN | | | | | |
| CD3-18 | VH | RYTMH | 128 | YINPSRG | 155 | YYDDH | 172 |
| | | | | YTNYNQK | | YCLDY | |
| | | | | VKD | | | |
| | VL | SASSS | 129 | DTSKLAS | 149 | QQWSS | 173 |
| | | VSYM | | | | NPFT | |
| | | N | | | | | |
| CD3-19 | VH | RYTMH | 128 | YINPSRG | 155 | YYDDH | 187 |
| | | | | YTNYNQK | | YSLDY | |
| | | | | VKD | | | |
| | VL | SASSS | 129 | DTSKLAS | 149 | QQWSS | 173 |
| | | VSYM | | | | NPFT | |
| | | N | | | | | |
| CD3-20 | VH | GYTMN | 141 | LINPYKG | 161 | SGYYGD | 188 |
| | | | | VSTYNQK | | SDWYF | |
| | | | | FKD | | DV | |
| | VL | RASQD | 142 | YTSRLH | 162 | QQGNTL | 189 |
| | | IRNY | | | | PWT | |
| | | LN | | | | | |
| CD3-21 | VH | TYAMN | 130 | RIRSKYN | 150 | HGNFGN | 174 |
| | | | | NYATYYA | | SYVSW | |
| | | | | DSVKD | | FAY | |
| | VL | RSSTG | 131 | GTNKRAP | 151 | ALWYS | 175 |
| | | AVTT | | | | NLWV | |
| | | SNYAN | | | | | |
| CD3-22 | VH | TYAMN | 130 | RIRSKYN | 159 | HGNFGD | 185 |
| | | | | NYATYY | | SYVS | |
| | | | | ADSVKG | | WFAY | |
| | VL | GSST | 140 | GTNKRAP | 151 | ALWYS | 186 |
| | | GAVT | | | | NHWV | |
| | | TSNY | | | | | |
| | | AN | | | | | |
| CD3-23 | VH | TYAMN | 130 | RIRSKAN | 163 | HGNFG | 185 |
| | | | | NYATYY | | DSYVS | |
| | | | | ADSV | | WFAY | |
| | | | | KG | | | |
| | VL | GSST | 140 | GTNK | 151 | ALWYS | 186 |
| | | GAVT | | RAP | | NHWV | |
| | | TSNY | | | | | |
| | | AN | | | | | |
| CD3-24 | VH | TYAMN | 130 | RIRSK | 159 | HGNFG | 190 |
| | | | | YNNYA | | DEYVS | |
| | | | | TYYAD | | WFAY | |
| | | | | SVKG | | | |
| | VL | GSST | 140 | GTNK | 151 | ALWYS | 186 |
| | | GAVT | | RAP | | NHWV | |
| | | TSNY | | | | | |
| | | AN | | | | | |
| CD3-25 | VH | TYAMN | 130 | RIRSKYN | 159 | HGNFG | 191 |
| | | | | NYATYY | | DPYVS | |
| | | | | ADSVKG | | WFAY | |
| | VL | GSST | 140 | GTNKRAP | 151 | ALWYS | 186 |
| | | GAVT | | | | NHWV | |
| | | TSNY | | | | | |
| | | AN | | | | | |
| CD3-26 | VH | TYAMN | 130 | RIRSKYN | 159 | HGNFG | 192 |
| | | | | NYATYY | | DSYVS | |
| | | | | ADSVKG | | WFDY | |
| | VL | GSST | 140 | GTNKRAP | 151 | ALWYS | 186 |
| | | GAVT | | | | NHWV | |
| | | TSNY | | | | | |
| | | AN | | | | | |
| CD3-27 | VH | TYAMS | 143 | RIRSKYN | 159 | HGNFG | 185 |
| | | | | NYATYY | | DSYVS | |
| | | | | ADSVKG | | WFAY | |
| | VL | GSST | 140 | GTNKRAP | 151 | ALWYS | 186 |
| | | GAVT | | | | NHWV | |
| | | TSNY | | | | | |
| | | AN | | | | | |
| CD3-28 | VH | TYAMN | 130 | RIRSKYN | 159 | HGNFG | 174 |
| | | | | NYATYY | | NSYVS | |
| | | | | ADSVKG | | WFAY | |
| | VL | GSSTG | 140 | GTNKRAP | 151 | ALWYS | 175 |
| | | AVTTS | | | | NLWV | |
| | | NYAN | | | | | |
| CD3-29 | VH | TYAMN | 130 | RIRSKYN | 159 | HGNFG | 174 |
| | | | | NYATYY | | NSYVS | |
| | | | | ADSVKG | | WFAY | |
| | VL | GSSTG | 140 | GTNKRAP | 151 | ALWYS | 175 |
| | | AVTTS | | | | NLWV | |
| | | NYAN | | | | | |
| CD3-30 | VH | TYAMN | 130 | RIRSKYN | 159 | HGNFG | 174 |
| | | | | NYATYY | | NSYVS | |
| | | | | ADSVKG | | WFAY | |
| | VL | GSST | 140 | GTNKRAP | 151 | ALWYS | 175 |
| | | GAVT | | | | NLWV | |
| | | TSNY | | | | | |
| | | AN | | | | | |
| CD3-31 | VH | TYAMN | 130 | RIRSKYN | 159 | HGNFG | 174 |
| | | | | NYATYY | | NSYVS | |
| | | | | ADSVKG | | WFAY | |
| | VL | GSST | 140 | GTNKRAP | 151 | ALWYS | 175 |
| | | GAVT | | | | NLWV | |
| | | TSNY | | | | | |
| | | AN | | | | | |
| CD3-32 | VH | TYAMN | 130 | RIRSKYN | 159 | HGNFG | 174 |
| | | | | NYATYY | | NSYVS | |
| | | | | ADSVKG | | WFAY | |
| | VL | GSST | 140 | GTNKRAP | 151 | ALWYS | 175 |
| | | GAVT | | | | NLWV | |
| | | TSNY | | | | | |
| | | AN | | | | | |
| CD3-33 | VH | TYAMN | 130 | RIRSKYN | 159 | HGNFG | 174 |
| | | | | NYATYY | | NSYVS | |
| | | | | ADSVKG | | WFAY | |
| | VL | GSST | 140 | GTNKRAP | 151 | ALWYS | 175 |
| | | GAVT | | | | NLWV | |
| | | TSNY | | | | | |
| | | AN | | | | | |
| CD3-34 | VH | TYAMN | 130 | RIRSKYN | 159 | HGNFG | 174 |
| | | | | NYATYY | | NSYVS | |
| | | | | ADSVKG | | WFAY | |
| | VL | GSST | 140 | GTNKRAP | 151 | ALWYS | 175 |
| | | GAVT | | | | NLWV | |
| | | TSNY | | | | | |
| | | AN | | | | | |
| CD3-35 | VH | TYAMH | 144 | RIRSKYN | 159 | HGNFG | 174 |
| | | | | NYATYY | | NSYVS | |
| | | | | ADSVKG | | WFAY | |
| | VL | GSST | 140 | GTNKRAP | 151 | ALWYS | 175 |
| | | GAVT | | | | NLWV | |
| | | TSNY | | | | | |
| | | AN | | | | | |
| CD3-36 | VH | TYAMS | 143 | RIRSKYN | 159 | HGNFG | 174 |
| | | | | NYATYY | | NSYVS | |
| | | | | ADSVKG | | WFAY | |
| | VL | GSSTGA | 140 | GTNKRAP | 151 | ALWYS | 175 |
| | | VTTSNY | | | | NLWV | |
| | | AN | | | | | |
| CD3-37 | VH | TYAMN | 130 | RIRSKYN | 159 | HGNFG | 174 |
| | | | | NYATYY | | NSYVS | |
| | | | | ADSVKG | | WFAY | |
| | VL | GSST | 140 | GTNKRAP | 151 | ALWYS | 175 |
| | | GAVT | | | | NLWV | |
| | | TSNY | | | | | |
| | | AN | | | | | |
| CD3-38 | VH | TYAMN | 130 | RIRSKAN | 164 | HGNFG | 174 |
| | | | | NYYATY | | NSYVS | |
| | | | | YADSVKG | | WFAY | |
| | VL | GSST | 140 | GTNKRAP | 151 | ALWYS | 175 |
| | | GAVT | | | | NLWV | |
| | | TSNY | | | | | |
| | | AN | | | | | |
| CD3-39 | VH | TYAMN | 130 | RIRSKAN | 165 | HGNFG | 174 |
| | | | | SYATYY | | NSYVS | |
| | | | | ADSVKG | | WFAY | |
| | VL | GSST | 140 | GTNKRAP | 151 | ALWYS | 175 |
| | | GAVT | | | | NLWV | |
| | | TSNY | | | | | |
| | | AN | | | | | |
| CD3-40 | VH | TYAMN | 130 | RIRSKYN | 166 | HGNFG | 174 |
| | | | | NYATAY | | NSYVS | |
| | | | | ADSVKG | | WFAY | |
| | VL | GSST | 140 | GTNKRAP | 151 | ALWYS | 175 |
| | | GAVT | | | | NLWV | |
| | | TSNY | | | | | |
| | | AN | | | | | |
| CD3-41 | VH | TYAMN | 130 | RIRSKYN | 159 | HGNFG | 174 |
| | | | | NYATYY | | NSYVS | |
| | | | | ADSVKG | | WFAY | |
| | VL | GSST | 140 | GTNKRAP | 151 | ALWYS | 175 |
| | | GAVT | | | | NLWV | |
| | | TSNY | | | | | |
| | | AN | | | | | |
| CD3-42 | VH | TYAMN | 130 | RIRSKYN | 159 | HGNFG | 174 |
| | | | | NYATYY | | NSYVS | |
| | | | | ADSVKG | | WFAY | |
| | VL | GSST | 140 | GTNKRAP | 151 | ALWYS | 175 |
| | | GAVT | | | | NLWV | |
| | | TSNY | | | | | |
| | | AN | | | | | |
| CD3-43 | VH | TYAMN | 130 | RIRSKYN | 159 | HGNFG | 174 |
| | | | | NYATYY | | NSYVS | |
| | | | | ADSVKG | | WFAY | |
| | VL | GSST | 140 | GTNKRAP | 151 | ALWYS | 175 |
| | | GAVT | | | | NLWV | |
| | | TSNY | | | | | |
| | | AN | | | | | |
| CD3-44 | VH | TYAMN | 130 | RIRSKYN | 159 | HGNFG | 174 |
| | | | | NYATYY | | NSYVS | |
| | | | | ADSVKG | | WFAY | |
| | VL | GSST | 140 | GTNKRAP | 151 | ALWYS | 175 |
| | | GAVT | | | | NLWV | |
| | | TSNY | | | | | |
| | | AN | | | | | |
| CD3-45 | VH | TYAMN | 130 | RIRSKYN | 159 | HGNFG | 174 |
| | | | | NYATYY | | NSYVS | |
| | | | | ADSVKG | | WFAY | |
| | VL | GSST | 140 | GTNKRAP | 151 | ALWYS | 175 |
| | | GAVT | | | | NLWV | |
| | | TSNY | | | | | |
| | | AN | | | | | |
| CD3-46 | VH | TYAMN | 130 | RIRSKYN | 159 | HGNFG | 174 |
| | | | | NYATYY | | NSYVS | |
| | | | | ADSVKG | | WFAY | |
| | VL | GSST | 140 | GTNKRAP | 151 | ALWYS | 175 |
| | | GAVT | | | | NLWV | |
| | | TSNY | | | | | |
| | | AN | | | | | |
| CD3-47 | VH | TYAMN | 130 | RIRSKYN | 159 | HGNFG | 174 |
| | | | | NYATYY | | NSYVS | |
| | | | | ADSVKG | | WFAY | |
| | VL | GSST | 140 | GTNKRAP | 151 | ALWYS | 175 |
| | | GAVT | | | | NLWV | |
| | | TSNY | | | | | |
| | | AN | | | | | |
| CD3-48 | VH | TYAMN | 130 | RIRSKYN | 159 | HGNFG | 174 |
| | | | | NYATYY | | NSYVS | |
| | | | | ADSVKG | | WFAY | |
| | VL | GSST | 140 | GTNKRAP | 151 | ALWYS | 175 |
| | | GAVT | | | | NLWV | |
| | | TSNY | | | | | |
| | | AN | | | | | |
| CD3-49 | VH | TYAMN | 130 | RIRSKYN | 159 | HGNFG | 174 |
| | | | | NYATYY | | NSYVS | |
| | | | | ADSVKG | | WFAY | |
| | VL | GSST | 140 | GTNKRAP | 151 | ALWYS | 175 |
| | | GAVT | | | | NLWV | |
| | | TSNY | | | | | |
| | | AN | | | | | |
| CD3-50 | VH | TYAMN | 130 | RIRSKYN | 159 | HGNFG | 185 |
| | | | | NYATYY | | DSYVS | |
| | | | | ADSVKG | | WFAY | |
| | VL | GSST | 140 | GTNKRAP | 151 | ALWYS | 175 |
| | | GAVT | | | | NLWV | |
| | | TSNY | | | | | |
| | | AN | | | | | |
| CD3-51 | VH | TYAMN | 130 | RIRSKYN | 159 | HGNFG | 193 |
| | | | | NYATYY | | QSYVS | |
| | | | | ADSVKG | | WFAY | |
| | VL | GSST | 140 | GTNKRAP | 151 | ALWYS | 175 |
| | | GAVT | | | | NLWV | |
| | | TSNY | | | | | |
| | | AN | | | | | |
| CD3-52 | VH | TYAMN | 130 | RIRSKYN | 159 | HGNFG | 174 |
| | | | | NYATYY | | NSYVS | |
| | | | | ADSVKG | | WFAY | |
| | VL | GSST | 140 | GTNKRAP | 151 | ALWYS | 175 |
| | | GAVT | | | | NLWV | |
| | | TSNY | | | | | |
| | | AN | | | | | |
| CD3-53 | VH | TYAMN | 130 | RIRSKYN | 159 | HGNFG | 194 |
| | | | | NYATYY | | NSYVS | |
| | | | | ADSVKG | | WFDY | |
| | VL | GSST | 140 | GTNKRAP | 151 | ALWYS | 175 |
| | | GAVT | | | | NLWV | |
| | | TSNY | | | | | |
| | | AN | | | | | |
| CD3-54 | VH | TYAMN | 130 | RIRSKYN | 159 | HGNFG | 174 |
| | | | | NYATYY | | NSYVS | |
| | | | | ADSVKG | | WFAY | |
| | VL | GSST | 140 | GTNKRAP | 151 | ALWYS | 175 |
| | | GAVT | | | | NLWV | |
| | | TSNY | | | | | |
| | | AN | | | | | |
| CD3-55 | VH | TYAMN | 130 | RIRSKYN | 159 | HGNFG | 174 |
| | | | | NYATYY | | NSYVS | |
| | | | | ADSVKG | | WFAY | |
| | VL | GSST | 140 | GTNKRAP | 151 | ALWYS | 175 |
| | | GAVT | | | | NLWV | |
| | | TSNY | | | | | |
| | | AN | | | | | |
| CD3-56 | VH | TYAMN | 130 | RIRSKYN | 159 | HGNFG | 174 |
| | | | | NYATYY | | NSYVS | |
| | | | | ADSVKG | | WFAY | |
| | VL | GSST | 140 | GTNKRAP | 151 | ALWYS | 175 |
| | | GAVT | | | | NLWV | |
| | | TSNY | | | | | |
| | | AN | | | | | |
| CD3-57 | VH | TYAMN | 130 | RIRSKYN | 159 | HGNFG | 174 |
| | | | | NYATYY | | NSYVS | |
| | | | | ADSVKG | | WFAY | |
| | VL | GSST | 140 | GTNKRAP | 151 | ALWYS | 175 |
| | | GAVT | | | | NLWV | |
| | | TSNY | | | | | |
| | | AN | | | | | |
| CD3-58 | VH | TYAMN | 130 | RIRSKYN | 159 | HGNFG | 174 |
| | | | | NYATYY | | NSYVS | |
| | | | | ADSVKG | | WFAY | |
| | VL | GSST | 140 | GTNKRAP | 151 | ALWYS | 175 |
| | | GAVT | | | | NLWV | |
| | | TSNY | | | | | |
| | | AN | | | | | |
| CD3-59 | VH | TYAMN | 130 | RIRSKYN | 159 | HGNFG | 174 |
| | | | | NYATYY | | NSYVS | |
| | | | | ADSVKG | | WFAY | |
| | VL | GSST | 140 | GTNKRAP | 151 | ALWYS | 175 |
| | | GAVT | | | | NLWV | |
| | | TSNY | | | | | |
| | | AN | | | | | |
| CD3-60 | VH | TYAMN | 130 | RIRSKYN | 159 | HGNFG | 174 |
| | | | | NYATYY | | NSYVS | |
| | | | | ADSVKG | | WFAY | |
| | VL | GSST | 145 | GTNKRAP | 151 | ALWYS | 175 |
| | | GAVT | | | | NLWV | |
| | | SSNY | | | | | |
| | | AN | | | | | |
| CD3-61 | VH | TYAMN | 130 | RIRSKYN | 159 | HGNFG | 174 |
| | | | | NYATYY | | NSYVS | |
| | | | | ADSVKG | | WFAY | |
| | VL | GSST | 146 | GTNKRAP | 151 | ALWYS | 175 |
| | | GAVT | | | | NLWV | |
| | | SGHY | | | | | |
| | | AN | | | | | |
| CD3-62 | VH | TYAMN | 130 | RIRSKYN | 159 | HGNFG | 174 |
| | | | | NYATYY | | NSYVS | |
| | | | | ADSVKG | | WFAY | |
| | VL | GSST | 140 | DTNKRAP | 167 | ALWYS | 175 |
| | | GAVT | | | | NLWV | |
| | | TSNY | | | | | |
| | | AN | | | | | |
| CD3-63 | VH | TYAMN | 130 | RIRSKYN | 159 | HGNFG | 174 |
| | | | | NYATYY | | NSYVS | |
| | | | | ADSVKG | | WFAY | |
| | VL | GSST | 140 | GTNNRAP | 168 | ALWYS | 175 |
| | | GAVT | | | | NLWV | |
| | | TSNY | | | | | |
| | | AN | | | | | |
| CD3-64 | VH | TYAMN | 130 | RIRSKYN | 159 | HGNFG | 174 |
| | | | | NYATYY | | NSYVS | |
| | | | | ADSVKG | | WFAY | |
| | VL | GSST | 140 | GTNKRAS | 169 | ALWYS | 175 |
| | | GAVT | | | | NLWV | |
| | | TSNY | | | | | |
| | | AN | | | | | |
| CD3-65 | VH | TYAMN | 130 | RIRSKYN | 159 | HGNFG | 174 |
| | | | | NYATYY | | NSYVS | |
| | | | | ADSVKG | | WFAY | |
| | VL | GSST | 140 | GTSNKHS | 170 | ALWYS | 175 |
| | | GAVT | | | | NLWV | |
| | | TSNY | | | | | |
| | | AN | | | | | |
| CD3-66 | VH | TYAMN | 130 | RIRSKYN | 159 | HGNFG | 174 |
| | | | | NYATYY | | NSYVS | |
| | | | | ADSVKG | | WFAY | |
| | VL | GSST | 140 | GTNKRAP | 151 | ALWYS | 175 |
| | | GAVT | | | | NLWV | |
| | | TSNY | | | | | |
| | | AN | | | | | |
| CD3-67 | VH | TYAMN | 130 | RIRSKYN | 159 | HGNFG | 174 |
| | | | | NYATYY | | NSYVS | |
| | | | | ADSVKG | | WFAY | |
| | VL | GSST | 140 | GTNKRAP | 151 | ALWYS | 175 |
| | | GAVT | | | | NLWV | |
| | | TSNY | | | | | |
| | | AN | | | | | |
| CD3-68 | VH | TYAMN | 130 | RIRSKYN | 159 | HGNFG | 174 |
| | | | | NYATYY | | NSYVS | |
| | | | | ADSVKG | | WFAY | |
| | VL | GSST | 140 | GTNKRAP | 151 | ALWYS | 175 |
| | | GAVT | | | | NLWV | |
| | | TSNY | | | | | |
| | | AN | | | | | |
| CD3-69 | VH | TYAMN | 130 | RIRSKYN | 159 | HGNFG | 174 |
| | | | | NYATYY | | NSYVS | |
| | | | | ADSVKG | | WFAY | |
| | VL | GSST | 140 | GTNK | 151 | ALWYS | 175 |
| | | GAVT | | RAP | | NLWV | |
| | | TSNY | | | | | |
| | | AN | | | | | |
| CD3-70 | VH | TYAMN | 130 | RIRSKYN | 159 | HGNFG | 174 |
| | | | | NYATYY | | NSYVS | |
| | | | | ADSVKG | | WFAY | |
| | VL | GSST | 140 | GTNKRAP | 151 | ALWYS | 175 |
| | | GAVT | | | | NLWV | |
| | | TSNY | | | | | |
| | | AN | | | | | |
| CD3-71 | VH | TYAMN | 130 | RIRSKYN | 159 | HGNFG | 174 |
| | | | | NYATYY | | NSYVS | |
| | | | | ADSVKG | | WFAY | |
| | VL | GSST | 140 | GTNKRAP | 151 | ALWYS | 175 |
| | | GAVT | | | | NLWV | |
| | | TSNY | | | | | |
| | | AN | | | | | |
| CD3-72 | VH | TYAMN | 130 | RIRSKYN | 159 | HGNFG | 174 |
| | | | | NYATYY | | NSYVS | |
| | | | | ADSVKG | | WFAY | |
| | VL | GSST | 140 | GTNKRAP | 151 | ALWYS | 175 |
| | | GAVT | | | | NLWV | |
| | | TSNY | | | | | |
| | | AN | | | | | |
| CD3-73 | VH | TYAMN | 130 | RIRSKYN | 159 | HGNFG | 174 |
| | | | | NYATYY | | NSYVS | |
| | | | | ADSVKG | | WFAY | |
| | VL | GSST | 140 | GTNKRAP | 151 | LLWYS | 195 |
| | | GAVT | | | | NLWV | |
| | | TSNY | | | | | |
| | | AN | | | | | |
| CD3-74 | VH | TYAMN | 130 | RIRSKYN | 159 | HGNFG | 174 |
| | | | | NYATYY | | NSYVS | |
| | | | | ADSVKG | | WFAY | |
| | VL | GSST | 140 | GTNKRAP | 151 | ALWYS | 186 |
| | | GAVT | | | | NHWV | |
| | | TSNY | | | | | |
| | | AN | | | | | |
| CD3-75 | VH | TYAMN | 130 | RIRSKYN | 159 | HGNFG | 174 |
| | | | | NYATYY | | NSYVS | |
| | | | | ADSVKG | | WFAY | |
| | VL | GSST | 140 | GTNKRAP | 151 | ALWYS | 175 |
| | | GAVT | | | | NLWV | |
| | | TSNY | | | | | |
| | | AN | | | | | |
| CD3-76 | VH | TYAMN | 130 | RIRSKYN | 159 | HGNFG | 174 |
| | | | | NYATYY | | NSYVS | |
| | | | | ADSVKG | | WFAY | |
| | VL | RSST | 131 | GTNKRAP | 151 | ALWYS | 175 |
| | | GAVT | | | | NLWV | |
| | | TSNY | | | | | |
| | | AN | | | | | |
| CD3-77 | VH | TYAMN | 130 | RIRSKYN | 159 | HGNFG | 174 |
| | | | | NYATYY | | NSYVS | |
| | | | | ADSVKG | | WFAY | |
| | VL | KSST | 147 | GTNKRAP | 151 | ALWYS | 175 |
| | | GAVT | | | | NLWV | |
| | | TSNY | | | | | |
| | | AN | | | | | |
| CD3-78 | VH | TYAMN | 130 | RIRSKYN | 159 | HGNFG | 174 |
| | | | | NYATYY | | NSYVS | |
| | | | | ADSVKG | | WFAY | |
| | VL | GSST | 140 | GTNKRAP | 151 | ALWYS | 186 |
| | | GAVT | | | | NHWV | |
| | | TSNY | | | | | |
| | | AN | | | | | |
| CD3-79 | VH | TYAMN | 130 | RIRSKYN | 159 | HGNFG | 174 |
| | | | | NYATYY | | NSYVS | |
| | | | | ADSVKG | | WFAY | |
| | VL | GSSTG | 140 | GTNKRAP | 151 | ALWYS | 175 |
| | | AVTTS | | | | NLWV | |
| | | NYAN | | | | | |
| CD3-80 | VH | TYAMN | 130 | RIRSKYN | 159 | HGNFG | 174 |
| | | | | NYATYY | | NSYVS | |
| | | | | ADSVKG | | WFAY | |
| | VL | GSST | 140 | GTNKRAP | 151 | ALWYS | 175 |
| | | GAVT | | | | NLWV | |
| | | TSNY | | | | | |
| | | AN | | | | | |
| CD3-81 | VH | TYAMN | 130 | RIRSKYN | 159 | HGNFG | 174 |
| | | | | NYATYY | | NSYVS | |
| | | | | ADSVKG | | WFAY | |
| | VL | GSST | 140 | GTNKRAP | 151 | ALWYS | 175 |
| | | GAVT | | | | NLWV | |
| | | TSNY | | | | | |
| | | AN | | | | | |
| CD3-82 | VH | TYAMN | 130 | RIRSKYN | 159 | HGNFG | 174 |
| | | | | NYATYY | | NSYVS | |
| | | | | ADSVKG | | WFAY | |
| | VL | GSST | 140 | GTNKRAP | 151 | ALWYS | 175 |
| | | GAVT | | | | NLWV | |
| | | TSNY | | | | | |
| | | AN | | | | | |
| CD3-83 | VH | TYAMN | 130 | RIRSKYN | 159 | HGNFG | 174 |
| | | | | NYATYY | | NSYVS | |
| | | | | ADSVKG | | WFAY | |
| | VL | GSSTGA | 140 | GTNKRAP | 151 | ALWYS | 175 |
| | | VTTSNY | | | | NLWV | |
| | | AN | | | | | |
| CD3-84 | VH | TYAMN | 130 | RIRSKYN | 159 | HGNFG | 174 |
| | | | | NYATYY | | NSYVS | |
| | | | | ADSVKG | | WFAY | |
| | VL | GSST | 140 | GTNKRAP | 151 | ALWYS | 175 |
| | | GAVT | | | | NLWV | |
| | | TSNY | | | | | |
| | | AN | | | | | |
| CD3-85 | VH | TYAMN | 130 | RIRSKYN | 159 | HGNFG | 174 |
| | | | | NYATYY | | NSYVS | |
| | | | | ADSVKG | | WFAY | |
| | VL | GSST | 140 | GTNKRAP | 151 | ALWYS | 175 |
| | | GAVT | | | | NLWV | |
| | | TSNY | | | | | |
| | | AN | | | | | |
| CD3-86 | VH | TYAMN | 130 | RIRSKYN | 159 | HGNFG | 174 |
| | | | | NYATYY | | NSYVS | |
| | | | | ADSVKG | | WFAY | |
| | VL | GSST | 140 | GTNKRAP | 151 | ALWYS | 175 |
| | | GAVT | | | | NLWV | |
| | | TSNY | | | | | |
| | | AN | | | | | |
| CD3-87 | VH | TYAMN | 130 | RIRSKYN | 159 | HGNFG | 185 |
| | | | | NYATYY | | DSYVS | |
| | | | | ADSVKG | | WFAY | |
| | VL | GSST | 140 | GTNKRAP | 151 | ALWYS | 175 |
| | | GAVT | | | | NLWV | |
| | | TSNY | | | | | |
| | | AN | | | | | |
| CD3-88 | VH | TYAMN | 130 | RIRSKYN | 159 | HGNFG | 185 |
| | | | | NYATYY | | DSYVS | |
| | | | | ADSVKG | | WFAY | |
| | VL | GSST | 140 | GTNKRAP | 151 | ALWYS | 175 |
| | | GAVT | | | | NLWV | |
| | | TSNY | | | | | |
| | | AN | | | | | |
| CD3-89 | VH | TYAMN | 130 | RIRSKAN | 163 | HGNFG | 185 |
| | | | | NYATYY | | DSYVS | |
| | | | | ADSVKG | | WFAY | |
| | VL | GSST | 140 | GTNKRAP | 151 | ALWYS | 175 |
| | | GAVT | | | | NLWV | |
| | | TSN | | | | | |
| | | YAN | | | | | |
| CD3-90 | VH | TYAMN | 130 | RIRSKYN | 159 | HGNFG | 192 |
| | | | | NYATYY | | DSYVS | |
| | | | | ADSVKG | | WFDY | |
| | VL | GSST | 140 | GTNKRAP | 151 | ALWYS | 175 |
| | | GAVT | | | | NLWV | |
| | | TSNY | | | | | |
| | | AN | | | | | |
| CD3-91 | VH | TYAMS | 143 | RIRSKAN | 163 | HGNFG | 192 |
| | | | | NYATYY | | DSYVS | |
| | | | | ADSVKG | | WFDY | |
| | VL | GSST | 140 | GTNKRAP | 151 | ALWYS | 175 |
| | | GAVT | | | | NLWV | |
| | | TSNY | | | | | |
| | | AN | | | | | |
| CD3-92 | VH | TYAMN | 130 | RIRSNGG | 171 | HGNFG | 174 |
| | | | | YSTYYA | | NSYVS | |
| | | | | DSVKG | | WFAY | |
| | VL | GSST | 140 | GTNKRAP | 151 | ALWYS | 175 |
| | | GAVT | | | | NLWV | |
| | | TSNY | | | | | |
| | | AN | | | | | |
| CD3-93 | VH | TYAMN | 130 | RIRSKYN | 159 | HGNFG | 174 |
| | | | | NYATYY | | NSYVS | |
| | | | | ADSVKG | | WFAY | |
| | VL | GSST | 140 | GTNKRAP | 151 | ALWYS | 175 |
| | | GAVT | | | | NLWV | |
| | | TSNY | | | | | |
| | | AN | | | | | |
| CD3-94 | VH | TYAMN | 130 | RIRSKYN | 159 | HGNFG | 174 |
| | | | | NYATYY | | NSYVS | |
| | | | | ADSVKG | | WFAY | |
| | VL | GSST | 140 | GTNKRAP | 151 | ALWYS | 175 |
| | | GAVT | | | | NLWV | |
| | | TSNY | | | | | |
| | | AN | | | | | |
| CD3-95 | VH | TYAMN | 130 | RIRSKYN | 159 | HGNFG | 174 |
| | | | | NYATYY | | NSYVS | |
| | | | | ADSVKG | | WFAY | |
| | VL | GSST | 140 | GTNKRAP | 151 | ALWYS | 175 |
| | | GAVT | | | | NLWV | |
| | | TSNY | | | | | |
| | | AN | | | | | |
| CD3-96 | VH | TYAMN | 130 | RIRSKYN | 159 | HGNFG | 174 |
| | | | | NYATYY | | NSYVS | |
| | | | | ADSVKG | | WFAY | |
| | VL | GSST | 140 | GTNKRAP | 151 | ALWYS | 186 |
| | | GAVT | | | | NHWV | |
| | | TSNY | | | | | |
| | | AN | | | | | |
| CD3-97 | VH | TYAMN | 130 | RIRSKYN | 159 | HGNFG | 185 |
| | | | | NYATYYA | | DSYVS | |
| | | | | DSVKG | | WFAY | |
| | VL | GSST | 140 | GTNK | 151 | ALWYS | 186 |
| | | GAVT | | RAP | | NHWV | |
| | | TSNY | | | | | |
| | | AN | | | | | |
| CD3-98 | VH | TYAMN | 130 | RIRSKYN | 159 | HGNFG | 174 |
| | | | | NYATYY | | NSYVS | |
| | | | | ADSVKG | | WFAY | |
| | VL | GSST | 140 | GTNKRAP | 151 | ALWYS | 186 |
| | | GAVT | | | | NHWV | |
| | | TSNY | | | | | |
| | | AN | | | | | |
| CD3-99 | VH | TYAMN | 130 | RIRSKYN | 159 | HGNFG | 174 |
| | | | | NYATYY | | NSYVS | |
| | | | | ADSVKG | | WFAY | |
| | VL | GSST | 140 | GTNKRAP | 151 | ALWYS | 186 |
| | | GAVT | | | | NHWV | |
| | | TSNY | | | | | |
| | | AN | | | | | |
| CD3-100 | VH | TYAMN | 130 | RIRSKYN | 159 | HGNFG | 185 |
| | | | | NYATYY | | DSYVS | |
| | | | | ADSVKG | | WFAY | |
| | VL | GSST | 140 | GTNKRAP | 151 | ALWYS | 186 |
| | | GAVT | | | | NHWV | |
| | | TSNY | | | | | |
| | | AN | | | | | |
| CD3-101 | VH | TYAMN | 130 | RIRSKYN | 159 | HGNFG | 185 |
| | | | | NYATYY | | DSYVS | |
| | | | | ADSVKG | | WFAY | |
| | VL | GSST | 140 | GTNKRAP | 151 | ALWYS | 186 |
| | | GAVT | | | | NHWV | |
| | | TSNY | | | | | |
| | | AN | | | | | |
| CD3-102 | VH | TYAMN | 130 | RIRSKYN | 159 | HGNFG | 174 |
| | | | | NYATYY | | NSYVS | |
| | | | | ADSVKG | | WFAY | |
| | VL | GSST | 140 | GTNKRAP | 151 | ALWYS | 186 |
| | | GAVT | | | | NHWV | |
| | | TSNY | | | | | |
| | | AN | | | | | |
| CD3-103 | VH | TYAMN | 130 | RIRSKYN | 159 | HGNFG | 174 |
| | | | | NYATYY | | NSYVS | |
| | | | | ADSVKG | | WFAY | |
| | VL | GSST | 140 | GTNKRAP | 151 | ALWYS | 186 |
| | | GAVT | | | | NHWV | |
| | | TSNY | | | | | |
| | | AN | | | | | |
| CD3-104 | VH | TYAMN | 130 | RIRSKYN | 159 | HGNFG | 185 |
| | | | | NYATYY | | DSYVS | |
| | | | | ADSVKG | | WFAY | |
| | VL | GSST | 140 | GTNKRAP | 151 | ALWYS | 186 |
| | | GAVT | | | | NHWV | |
| | | TSNY | | | | | |
| | | AN | | | | | |
| CD3-105 | VH | TYAMN | 130 | RIRSKYN | 159 | HGNFG | 185 |
| | | | | NYATYY | | DSYVS | |
| | | | | ADSVKG | | WFAY | |
| | VL | GSST | 140 | GTNKRAP | 151 | ALWYS | 186 |
| | | GAVT | | | | NHWV | |
| | | TSNY | | | | | |
| | | AN | | | | | |
| CD3-106 | VH | TYAMN | 130 | RIRSKYN | 159 | HGNFG | 174 |
| | | | | NYATYY | | NSYVS | |
| | | | | ADSVKG | | WFAY | |
| | VL | GSST | 140 | GTNKRAP | 151 | ALWY | 186 |
| | | GAVT | | | | SNHWV | |
| | | TSNY | | | | | |
| | | AN | | | | | |
| CD3-107 | VH | TYAMN | 130 | RIRSKYN | 159 | HGNFG | 174 |
| | | | | NYATYY | | NSYVS | |
| | | | | ADSVKG | | WFAY | |
| | VL | GSST | 140 | GTNKRAP | 151 | ALWYS | 186 |
| | | GAVT | | | | NHWV | |
| | | TSNY | | | | | |
| | | AN | | | | | |
| CD3-108 | VH | TYAMN | 130 | RIRSKYN | 159 | HGNFG | 185 |
| | | | | NYATYY | | DSYVS | |
| | | | | ADSVKG | | WFAY | |
| | VL | GSST | 140 | GTNKRAP | 151 | ALWYS | 186 |
| | | GAVT | | | | NHWV | |
| | | TSNY | | | | | |
| | | AN | | | | | |
| CD3-109 | VH | TYAMN | 130 | RIRSKYN | 159 | HGNFG | 185 |
| | | | | NYATYY | | DSYVS | |
| | | | | ADSVKG | | WFAY | |
| | VL | GSST | 140 | GTNKRAP | 151 | ALWYS | 186 |
| | | GAVT | | | | NHWV | |
| | | TSNY | | | | | |
| | | AN | | | | | |
| CD3-110 | VH | TYAMN | 130 | RIRSKYN | 159 | HGNFG | 174 |
| | | | | NYATYY | | NSYVS | |
| | | | | ADSVKG | | WFAY | |
| | VL | GSST | 140 | GTNKRAP | 151 | ALWYS | 186 |
| | | GAVT | | | | NHWV | |
| | | TSNY | | | | | |
| | | AN | | | | | |
| CD3-111 | VH | TYAMN | 130 | RIRSKYN | 159 | HGNFG | 174 |
| | | | | NYATYY | | NSYVS | |
| | | | | ADSVKG | | WFAY | |
| | VL | GSST | 140 | GTNKRAP | 151 | ALWYS | 186 |
| | | GAVT | | | | NHWV | |
| | | TSNY | | | | | |
| | | AN | | | | | |
| CD3-112 | VH | TYAMN | 130 | RIRSKYN | 159 | HGNFG | 185 |
| | | | | NYATYY | | DSYVS | |
| | | | | ADSVKG | | WFAY | |
| | VL | GSST | 140 | GTNKRAP | 151 | ALWYS | 186 |
| | | GAVT | | | | NHWV | |
| | | TSNY | | | | | |
| | | AN | | | | | |
| CD3-113 | VH | TYAMN | 130 | RIRSKYN | 159 | HGNFG | 185 |
| | | | | NYATYY | | DSYVS | |
| | | | | ADSVKG | | WFAY | |
| | VL | GSST | 140 | GTNKRAP | 151 | ALWYS | 186 |
| | | GAVT | | | | NHWV | |
| | | TSNY | | | | | |
| | | AN | | | | | |
| CD3-114 | VH | TYAMN | 130 | RIRSKYN | 159 | HGNFG | 174 |
| | | | | NYATYY | | NSYVS | |
| | | | | ADSVKG | | WFAY | |
| | VL | GSST | 140 | GTNKRAP | 151 | ALWYS | 186 |
| | | GAVT | | | | NHWV | |
| | | TSNY | | | | | |
| | | AN | | | | | |
| CD3-115 | VH | TYAMN | 130 | RIRSKYN | 159 | HGNFG | 174 |
| | | | | NYATYY | | NSYVS | |
| | | | | ADSVKG | | WFAY | |
| | VL | GSST | 140 | GTNKRAP | 151 | ALWYS | 186 |
| | | GAVT | | | | NHWV | |
| | | TSNY | | | | | |
| | | AN | | | | | |
| CD3-116 | VH | TYAMN | 130 | RIRSKYN | 159 | HGNFG | 185 |
| | | | | NYATYY | | DSYVS | |
| | | | | ADSVKG | | WFAY | |
| | VL | GSST | 140 | GTNKRAP | 151 | ALWYS | 186 |
| | | GAVT | | | | NHWV | |
| | | TSNY | | | | | |
| | | AN | | | | | |
| CD3-117 | VH | TYAMN | 130 | RIRSKYN | 159 | HGNFG | 185 |
| | | | | NYATYY | | DSYVS | |
| | | | | ADSVKG | | WFAY | |
| | VL | GSST | 140 | GTNKRAP | 151 | ALWYS | 186 |
| | | GAVT | | | | NHWV | |
| | | TSNY | | | | | |
| | | AN | | | | | |
| CD3-118 | VH | TYAMN | 130 | RIRSKYN | 159 | HGNFG | 174 |
| | | | | NYATYY | | NSYVS | |
| | | | | ADSVKG | | WFAY | |
| | VL | GSST | 140 | GTNKRAP | 151 | ALWYS | 186 |
| | | GAVT | | | | NHWV | |
| | | TSNY | | | | | |
| | | AN | | | | | |
| CD3-119 | VH | TYAMN | 130 | RIRSKYN | 159 | HGNFG | 174 |
| | | | | NYATYY | | NSYVS | |
| | | | | ADSVKG | | WFAY | |
| | VL | GSST | 140 | GTNKRAP | 151 | ALWYS | 186 |
| | | GAVT | | | | NHWV | |
| | | TSNY | | | | | |
| | | AN | | | | | |
| CD3-120 | VH | TYAMN | 130 | RIRSKYN | 159 | HGNFG | 185 |
| | | | | NYATYY | | DSYVS | |
| | | | | ADSVKG | | WFAY | |
| | VL | GSST | 140 | GTNKRAP | 151 | ALWYS | 186 |
| | | GAVT | | | | NHWV | |
| | | TSNY | | | | | |
| | | AN | | | | | |
| CD3-121 | VH | TYAMN | 130 | RIRSKYN | 159 | HGNFG | 185 |
| | | | | NYATYY | | DSYVS | |
| | | | | ADSVKG | | WFAY | |
| | VL | GSST | 140 | GTNKRAP | 151 | ALWYS | 186 |
| | | GAVT | | | | NHWV | |
| | | TSNY | | | | | |
| | | AN | | | | | |
| CD3-122 | VH | TYAMN | 130 | RIRSKYN | 159 | HGNFG | 174 |
| | | | | NYATYY | | NSYVS | |
| | | | | ADSVKG | | WFAY | |
| | VL | GSST | 140 | GTNKRAP | 151 | ALWYS | 186 |
| | | GAVT | | | | NHWV | |
| | | TSNY | | | | | |
| | | AN | | | | | |
| CD3-123 | VH | TYAMN | 130 | RIRSKYN | 159 | HGNFG | 174 |
| | | | | NYATYY | | NSYVS | |
| | | | | ADSVKG | | WFAY | |
| | VL | GSST | 140 | GTNKRAP | 151 | ALWYS | 186 |
| | | GAVT | | | | NHWV | |
| | | TSNY | | | | | |
| | | AN | | | | | |
| CD3-124 | VH | TYAMN | 130 | RIRSKYN | 159 | HGNFG | 185 |
| | | | | NYATYY | | DSYVS | |
| | | | | ADSVKG | | WFAY | |
| | VL | GSST | 140 | GTNKRAP | 151 | ALWYS | 186 |
| | | GAVT | | | | NHWV | |
| | | TSNY | | | | | |
| | | AN | | | | | |
| CD3-125 | VH | TYAMN | 130 | RIRSKYN | 159 | HGNFG | 185 |
| | | | | NYATYY | | DSYVS | |
| | | | | ADSVKG | | WFAY | |
| | VL | GSST | 140 | GTNKRAP | 151 | ALWYS | 186 |
| | | GAVT | | | | NHWV | |
| | | TSNY | | | | | |
| | | AN | | | | | |
| CD3-126 | VH | TYAMN | 130 | RIRSKYN | 159 | HGNFG | 174 |
| | | | | NYATYY | | NSYVS | |
| | | | | ADSVKG | | WFAY | |
| | VL | GSST | 140 | GTNKRAP | 151 | ALWYS | 186 |
| | | GAVT | | | | NHWV | |
| | | TSNY | | | | | |
| | | AN | | | | | |
| CD3-127 | VH | TYAMN | 130 | RIRSKYN | 159 | HGNFG | 174 |
| | | | | NYATYY | | NSYVS | |
| | | | | ADSVKG | | WFAY | |
| | VL | GSST | 140 | GTNKRAP | 151 | ALWYS | 186 |
| | | GAVT | | | | NHWV | |
| | | TSNY | | | | | |
| | | AN | | | | | |
| CD3-128 | VH | TYAMN | 130 | RIRSKYN | 159 | HGNFG | 185 |
| | | | | NYATYY | | DSYVS | |
| | | | | ADSVKG | | WFAY | |
| | VL | GSST | 140 | GTNKRAP | 151 | ALWYS | 186 |
| | | GAVT | | | | NHWV | |
| | | TSNY | | | | | |
| | | AN | | | | | |

TABLE 8C
CD3 Binders-CDR sequences according to Chothia numbering scheme

| Binding | | | SEQ | | SEQ | | SEQ |
|---|---|---|---|---|---|---|---|
| Domain | Chain | CDR1 | ID NO: | CDR2 | ID NO: | CDR3 | ID NO: |
| CD3-1 | VH | GYTFTRY | 196 | NPSRGY | 209 | YYDDHYCLDY | 172 |
| | VL | SSSVSY | 197 | DTS | 210 | WSSNPF | 221 |
| CD3-2 | VH | GFTFNTY | 198 | RSKYNNYA | 211 | HGNFGNSYVSWFAY | 174 |
| | VL | STGAVTTSNY | 199 | GTN | 212 | WYSNLW | 222 |
| CD3-3 | VH | GYTFTSY | 200 | NPSSGY | 213 | WQDYDVYFDY | 176 |
| | VL | SSSVSY | 197 | ATS | 214 | WSSNPP | 223 |
| CD3-4 | VH | GYTFTRY | 196 | NPSRGY | 209 | YYDDHYCLDY | 172 |
| | VL | SSSVSY | 197 | DTS | 210 | WSSNPL | 224 |
| CD3-5 | VH | GYTFTRY | 196 | NPSRGY | 209 | YYDDHYCLDY | 172 |
| | VL | SSSVSY | 197 | DTS | 210 | WSSNPF | 221 |
| CD3-6 | VH | GFKFSGY | 201 | WYDGSK | 215 | QMGYWHFDL | 179 |
| | VL | SQSVSSY | 202 | DAS | 216 | RSNWPPL | 225 |
| CD3-7 | VH | GFTFSTY | 203 | RSKYNNYAT | 217 | HGNFGNSYVSWFA | 226 |
| | VL | STGAVTTSNY | 199 | GTN | 212 | VVYSNLW | 222 |
| CD3-8 | VH | GYTFTRY | 196 | NPSRGY | 209 | YYDDHYCLDY | 172 |
| | VL | SSSVSY | 197 | DTS | 210 | WSSNPL | 224 |
| CD3-9 | VH | GFTFNTY | 198 | RSKYNNYAT | 217 | HGNFGNSYVSWFA | 226 |
| | VL | STGAVTTSNY | 199 | GTN | 212 | WYSNLW | 222 |
| CD3-10 | VH | GFTFNTY | 198 | RSKYNNYA | 211 | HGNFGNSYVSWFAY | 174 |
| | VL | STGAVTTSNY | 199 | GTN | 212 | WYSNLW | 222 |
| CD3-11 | VH | GFTFNSY | 204 | RSKYNNYA | 211 | HGNFGNSYVSWWAY | 182 |
| | VL | STGAVTSGNY | 205 | GTK | 218 | WYSNRW | 227 |
| CD3-12 | VH | GFTFNKY | 206 | YA | 211 | HGNFGNSYISYWAY | 184 |
| | VL | STGAVTSGNY | 205 | GTK | 218 | WYSNRW | 227 |
| CD3-13 | VH | GYTFTRY | 196 | NPSRGY | 209 | YYDDHYCLDY | 172 |
| | VL | SSSVSY | 197 | DTS | 210 | WSSNPF | 221 |
| CD3-14 | VH | GFTFSTY | 203 | RSKYNNYA | 211 | HGNFGNSYVSWFAY | 174 |
| | VL | STGAVTTSNY | 199 | GTN | 212 | WYSNLW | 222 |
| CD3-15 | VH | GFTFNTY | 198 | RSKYNNYA | 211 | HGNFGNSYVSWFAY | 174 |
| | VL | STGAVTTSNY | 199 | GTN | 212 | WYSNLW | 222 |
| CD3-16 | VH | GFTFNTY | 198 | RSKYNNYA | 211 | HGNFGNSYVSWFAY | 174 |
| | VL | STGAVTTSNY | 199 | GTN | 212 | WYSNLW | 222 |
| CD3-17 | VH | GFTFSTY | 203 | RSKYNNYA | 211 | HGNFGDSYVSWFAY | 185 |
| | VL | STGAVTTSNY | 199 | GTN | 212 | WYSNHW | 228 |
| CD3-18 | VH | GYTFTRY | 196 | NPSRGY | 209 | YYDDHYCLDY | 172 |
| | VL | SSSVSY | 197 | DTS | 210 | WSSNPF | 221 |
| CD3-19 | VH | GYTFTRY | 196 | NPSRGY | 209 | YYDDHYSLDY | 187 |
| | VL | SSSVSY | 197 | DTS | 210 | WSSNPF | 221 |
| CD3-20 | VH | GYSFTGY | 207 | NPYKGV | 219 | SGYYGDSDWYFDV | 188 |
| | VL | SQDIRNY | 208 | YTS | 220 | GNTLPW | 229 |
| CD3-21 | VH | GFTFNTY | 198 | RSKYNNYA | 211 | HGNFGNSYVSWFAY | 174 |
| | VL | RSSTGAVTTSNYAN | 131 | GTNKRAP | 151 | ALWYSNLWV | 175 |

TABLE 8D
CD3 Binders-CDR sequences according to combination of
Kabat and Chothia numbering schemes

| Binding | | | SEQ | | SEQ | | SEQ |
|---|---|---|---|---|---|---|---|
| Domain | Chain | CDR1 | ID NO: | CDR2 | ID NO: | CDR3 | ID NO: |
| CD3-1 | VH | GYTFTRYTMH | 230 | YINPSRGYTNYNQKFKD | 148 | YYDDHYCLDY | 172 |
| | VL | SASSSVSYMN | 129 | DTSKLAS | 149 | QQWSSNPFT | 173 |
| CD3-2 | VH | GFTFNTYAMN | 231 | RIRSKYNNYATYYADSVKD | 150 | HGNFGNSYVSWFAY | 174 |
| | VL | RSSTGAVTTSNYAN | 131 | GTNKRAP | 151 | ALWYSNLWV | 175 |
| CD3-3 | VH | GYTFTSYTMH | 232 | YINPSSGYTKYNQKFKD | 152 | WQDYDVYFDY | 176 |
| | VL | RASSSVSYMH | 133 | ATSNLAS | 153 | QQWSSNPPT | 177 |
| CD3-4 | VH | GYTFTRYTMH | 230 | YINPSRGYTNYNQKFKD | 148 | YYDDHYCLDY | 172 |
| | VL | RASSSVSYMN | 134 | DTSKVAS | 154 | QQWSSNPLT | 178 |
| CD3-5 | VH | GYTFTRYTMH | 230 | YINPSRGYTNYNQKVKD | 155 | YYDDHYCLDY | 172 |
| | VL | SASSSVSYMN | 129 | DTSKLAS | 149 | QQWSSNPFT | 173 |
| CD3-6 | VH | GFKFSGYGMH | 233 | VIVVYDGSKKYYVDSVKG | 156 | QMGYWHFDL | 179 |
| | VL | RASQSVSSYLA | 136 | DASNRAT | 157 | QQRSNWPPLT | 180 |
| CD3-7 | VH | GFTFSTYAMN | 234 | RIRSKYNNYATYYADSVK | 238 | HGNFGNSYVSWFAY | 174 |
| | VL | RSSTGAVTTSNYAN | 131 | GTNKRAP | 151 | ALWYSNLWV | 175 |
| CD3-8 | VH | GYTFTRYTMH | 230 | YINPSRGYTNYNQKFKD | 148 | YYDDHYCLDY | 172 |
| | VL | RASSSVSYMN | 134 | DTSKVAS | 154 | QQWSSNPLT | 178 |
| CD3-9 | VH | GFTFNTYAMN | 231 | RIRSKYNNYATYYADSVK | 238 | HGNFGNSYVSWFAY | 174 |
| | VL | RSSTGAVTTSNYAN | 131 | GTNKRAP | 151 | ALWYSNLWV | 175 |
| CD3-10 | VH | GFTFNTYAMN | 231 | RIRSKYNNYATYYADSVKD | 150 | HGNFGNSYVSWFAY | 174 |
| | VL | RSSTGAVTTSNYAN | 131 | GTNKRAP | 151 | ALWYSNLWV | 175 |
| CD3-11 | VH | GFTFNSYAMN | 235 | RIRSKYNNYATYYADSVKG | 159 | HGNFGNSYVSWFAY | 182 |
| | VL | GSSTGAVTSGNYPN | 138 | GTKFLAP | 160 | VLWYSNRWV | 183 |
| CD3-12 | VH | GFTFNKYAMN | 236 | RIRSKYNNYATYYADSVKD | 150 | HGNFGNSYISYWAY | 184 |
| | VL | GSSTGAVTSGNYPN | 138 | GTKFLAP | 160 | VLWYSNRWV | 183 |
| CD3-13 | VH | GYTFTRYTMH | 230 | YINPSRGYTNYNQKFKD | 148 | YYDDHYCLDY | 172 |
| | VL | SASSSVSYMN | 129 | DTSKLAS | 149 | QQWSSNPFT | 173 |
| CD3-14 | VH | GFTFSTYAMN | 234 | RIRSKYNNYATYYADSVKD | 150 | HGNFGNSYVSWFAY | 174 |
| | VL | RSSTGAVTTSNYAN | 131 | GTNKRAP | 151 | ALVVYSNLWV | 175 |
| CD3-15 | VH | GFTFNTYAMN | 231 | RIRSKYNNYATYYADSVKD | 150 | HGNFGNSYVSWFAY | 174 |
| | VL | RSSTGAVTTSNYAN | 131 | GTNKRAP | 151 | ALWYSNLWV | 175 |
| CD3-16 | VH | GFTFNTYAMN | 231 | RIRSKYNNYATYYADSVKG | 159 | HGNFGNSYVSWFAY | 174 |
| | VL | GSSTGAVTTSNYAN | 140 | GTNKRAP | 151 | ALWYSNLWV | 175 |
| CD3-17 | VH | GFTFSTYAMN | 234 | RIRSKYNNYATYYADSVKG | 159 | HGNFGDSYVSWFAY | 185 |
| | VL | GSSTGAVTTSNYAN | 140 | GTNKRAP | 151 | ALWYSNHWV | 186 |
| CD3-18 | VH | GYTFTRYTMH | 230 | YINPSRGYTNYNQKVKD | 155 | YYDDHYCLDY | 172 |
| | VL | SASSSVSYMN | 129 | DTSKLAS | 149 | QQWSSNPFT | 173 |
| CD3-19 | VH | GYTFTRYTMH | 230 | YINPSRGYTNYNQKVKD | 155 | YYDDHYSLDY | 187 |
| | VL | SASSSVSYMN | 129 | DTSKLAS | 149 | QQWSSNPFT | 173 |
| CD3-20 | VH | GYSFTGYTMN | 237 | LINPYKGVSTYNQKFKD | 161 | SGYYGDSDWYFDV | 188 |
| | VL | RASQDIRNYLN | 142 | YTSRLHS | 239 | QQGNTLPWT | 189 |

In some embodiments, a MBM (e.g., a TBM) of the disclosure can comprise a 003 ABM which comprises the CDRs of any of 003-1 to 003-128 as defined by Kabat numbering (e.g., as set forth in Table 8B). In other embodiments, a MBM (e.g., a TBM) of the disclosure can comprise a CD3 ABM which comprises the CDRs of any of CD3-1 to CD3-128 as defined by Chothia numbering (e.g., as set forth in Table 8C). In yet other embodiments, a MBM (e.g., a TBM) of the disclosure can comprise a CD3 ABM which comprises the CDRs of any of CD3-1 to CD3-128 as defined by a combination of Kabat and Chothia numbering (e.g., as set forth in Table 8D).

In some embodiments, a CD3 ABM comprises the CDR sequences of CD3-1. In some embodiments, a CD3 ABM comprises the CDR sequences of CD3-2. In some embodiments, a CD3 ABM comprises the CDR sequences of CD3-3. In some embodiments, a CD3 ABM comprises the CDR sequences of CD3-4. In some embodiments, a CD3 ABM comprises the CDR sequences of CD3-5. In some embodiments, a CD3 ABM comprises the CDR sequences of CD3-6. In some embodiments, a CD3 ABM comprises the CDR sequences of CD3-7. In some embodiments, a CD3 ABM comprises the CDR sequences of CD3-8. In some embodiments, a CD3 ABM comprises the CDR sequences of CD3-9. In some embodiments, a CD3 ABM comprises the CDR sequences of CD3-10. In some embodiments, a CD3 ABM comprises the CDR sequences of CD3-11. In some embodiments, a CD3 ABM comprises the CDR sequences of CD3-12. In some embodiments, a CD3 ABM comprises the CDR sequences of CD3-13. In some embodiments, a CD3 ABM comprises the CDR sequences of CD3-14. In some embodiments, a CD3 ABM comprises the CDR sequences of CD3-15. In some embodiments, a CD3 ABM comprises the CDR sequences of CD3-16. In some embodiments, a CD3 ABM comprises the CDR sequences of CD3-17. In some embodiments, a CD3 ABM comprises the CDR sequences of CD3-18. In some embodiments, a CD3 ABM comprises the CDR sequences of CD3-19. In some embodiments, a CD3 ABM comprises the CDR sequences of CD3-20. In some embodiments, a CD3 ABM comprises the CDR sequences of CD3-21. In some embodiments, a CD3 ABM comprises the CDR sequences of CD3-22. In some embodiments, a CD3 ABM comprises the CDR sequences of CD3-23. In some embodiments, a CD3 ABM comprises the CDR sequences of CD3-24. In some embodiments, a CD3 ABM comprises the CDR sequences of CD3-25. In some embodiments, a CD3 ABM comprises the CDR sequences of CD3-26. In some embodiments, a CD3 ABM comprises the CDR sequences of CD3-27. In some embodiments, a CD3 ABM comprises the CDR sequences of CD3-28. In some embodiments, a CD3 ABM comprises the CDR sequences of CD3-29. In some embodiments, a CD3 ABM comprises the CDR sequences of CD3-30. In some embodiments, a CD3 ABM comprises the CDR sequences of CD3-31. In some embodiments, a CD3 ABM comprises the CDR sequences of CD3-32. In some embodiments, a CD3 ABM comprises the CDR sequences of CD3-33. In some embodiments, a CD3 ABM comprises the CDR sequences of CD3-34. In some embodiments, a CD3 ABM comprises the CDR sequences of CD3-35. In some embodiments, a CD3 ABM comprises the CDR sequences of CD3-36. In some embodiments, a CD3 ABM comprises the CDR sequences of CD3-37. In some embodiments, a CD3 ABM comprises the CDR sequences of CD3-38. In some embodiments, a CD3 ABM comprises the CDR sequences of CD3-39. In some embodiments, a CD3 ABM comprises the CDR sequences of CD3-40. In some embodiments, a CD3 ABM comprises the CDR sequences of CD3-41. In some embodiments, a CD3 ABM comprises the CDR sequences of CD3-42. In some embodiments, a CD3 ABM comprises the CDR sequences of CD3-43. In some embodiments, a CD3 ABM comprises the CDR sequences of CD3-44. In some embodiments, a CD3 ABM comprises the CDR sequences of CD3-45. In some embodiments, a CD3 ABM comprises the CDR sequences of CD3-46. In some embodiments, a CD3 ABM comprises the CDR sequences of CD3-47. In some embodiments, a CD3 ABM comprises the CDR sequences of CD3-48. In some embodiments, a CD3 ABM comprises the CDR sequences of CD3-49. In some embodiments, a CD3 ABM comprises the CDR sequences of CD3-50. In some embodiments, a CD3 ABM comprises the CDR sequences of CD3-51. In some embodiments, a CD3 ABM comprises the CDR sequences of CD3-52. In some embodiments, a CD3 ABM comprises the CDR sequences of CD3-53. In some embodiments, a CD3 ABM comprises the CDR sequences of CD3-54. In some embodiments, a CD3 ABM comprises the CDR sequences of CD3-55. In some embodiments, a CD3 ABM comprises the CDR sequences of CD3-56. In some embodiments, a CD3 ABM comprises the CDR sequences of CD3-57. In some embodiments, a CD3 ABM comprises the CDR sequences of CD3-58. In some embodiments, a CD3 ABM comprises the CDR sequences of CD3-59. In some embodiments, a CD3 ABM comprises the CDR sequences of CD3-60. In some embodiments, a CD3 ABM comprises the CDR sequences of CD3-61. In some embodiments, a CD3 ABM comprises the CDR sequences of CD3-62. In some embodiments, a CD3 ABM comprises the CDR sequences of CD3-63. In some embodiments, a CD3 ABM comprises the CDR sequences of CD3-64. In some embodiments, a CD3 ABM comprises the CDR sequences of CD3-65. In some embodiments, a CD3 ABM comprises the CDR sequences of CD3-66. In some embodiments, a CD3 ABM comprises the CDR sequences of CD3-67. In some embodiments, a CD3 ABM comprises the CDR sequences of CD3-68. In some embodiments, a CD3 ABM comprises the CDR sequences of CD3-69. In some embodiments, a CD3 ABM comprises the CDR sequences of CD3-70. In some embodiments, a CD3 ABM comprises the CDR sequences of CD3-71. In some embodiments, a CD3 ABM comprises the CDR sequences of CD3-72. In some embodiments, a CD3 ABM comprises the CDR sequences of CD3-73. In some embodiments, a CD3 ABM comprises the CDR sequences of CD3-74. In some embodiments, a CD3 ABM comprises the CDR sequences of CD3-75. In some embodiments, a CD3 ABM comprises the CDR sequences of CD3-76. In some embodiments, a CD3 ABM comprises the CDR sequences of CD3-77. In some embodiments, a CD3 ABM comprises the CDR sequences of CD3-78. In some embodiments, a CD3 ABM comprises the CDR sequences of CD3-79. In some embodiments, a CD3 ABM comprises the CDR sequences of CD3-80. In some embodiments, a CD3 ABM comprises the CDR sequences of CD3-81. In some embodiments, a CD3 ABM comprises the CDR sequences of CD3-82. In some embodiments, a CD3 ABM comprises the CDR sequences of CD3-83. In some embodiments, a CD3 ABM comprises the CDR sequences of CD3-84. In some embodiments, a CD3 ABM comprises the CDR sequences of CD3-85. In some embodiments, a CD3 ABM comprises the CDR sequences of CD3-86. In some embodiments, a CD3 ABM comprises the CDR sequences of CD3-87. In some embodiments, a CD3 ABM comprises the CDR sequences of CD3-88. In some embodiments, a CD3 ABM comprises the CDR sequences of CD3-89. In some embodiments, a CD3 ABM comprises the CDR sequences of CD3-90. In some embodiments, a CD3 ABM comprises the CDR sequences of CD3-91. In some embodiments, a CD3 ABM comprises the CDR sequences of CD3-92. In some embodiments, a CD3 ABM comprises the CDR sequences of CD3-93. In some embodiments, a CD3 ABM comprises the CDR sequences of CD3-94. In some embodiments, a CD3 ABM comprises the CDR sequences of CD3-95. In some embodiments, a CD3 ABM comprises the CDR sequences of CD3-96. In some embodiments, a CD3 ABM comprises the CDR sequences of CD3-97. In some embodiments, a CD3 ABM comprises the CDR sequences of CD3-98. In some embodiments, a CD3 ABM comprises the CDR sequences of CD3-99. In some embodiments, a CD3 ABM comprises the CDR sequences of CD3-100. In some embodiments, a CD3 ABM comprises the CDR sequences of CD3-101. In some embodiments, a CD3 ABM comprises the CDR sequences of CD3-102. In some embodiments, a CD3 ABM comprises the CDR sequences of CD3-103. In some embodiments, a CD3 ABM comprises the CDR sequences of CD3-104. In some embodiments, a CD3 ABM comprises the CDR sequences of CD3-105. In some embodiments, a CD3 ABM comprises the CDR sequences of CD3-106. In some embodiments, a CD3 ABM comprises the CDR sequences of CD3-107. In some embodiments, a CD3 ABM comprises the CDR sequences of CD3-108. In some embodiments, a CD3 ABM comprises the CDR sequences of CD3-109. In some embodiments, a CD3 ABM comprises the CDR sequences of CD3-110. In some embodiments, a CD3 ABM comprises the CDR sequences of CD3-111. In some embodiments, a CD3 ABM comprises the CDR sequences of CD3-112. In some embodiments, a CD3 ABM comprises the CDR sequences of CD3-113. In some embodiments, a CD3 ABM comprises the CDR sequences of CD3-114. In some embodiments, a CD3 ABM comprises the CDR sequences of CD3-115. In some embodiments, a CD3 ABM comprises the CDR sequences of CD3-116. In some embodiments, a CD3 ABM comprises the CDR sequences of CD3-117. In some embodiments, a CD3 ABM comprises the CDR sequences of CD3-118. In some embodiments, a CD3 ABM comprises the CDR sequences of CD3-119. In some embodiments, a CD3 ABM comprises the CDR sequences of CD3-120. In some embodiments, a CD3 ABM comprises the CDR sequences of CD3-121. In some embodiments, a CD3 ABM comprises the CDR sequences of CD3-122. In some embodiments, a CD3 ABM comprises the CDR sequences of CD3-123. In some embodiments, a CD3 ABM comprises the CDR sequences of CD3-124. In some embodiments, a CD3 ABM comprises the CDR sequences of CD3-125. In some embodiments, a CD3 ABM comprises the CDR sequences of CD3-126. In some embodiments, a CD3 ABM comprises the CDR sequences of CD3-127. In some embodiments, a CD3 ABM comprises the CDR sequences of CD3-128.

A MBM (e.g., a TBM) of the disclosure can comprise the complete heavy and light variable sequences of any of CD3-1 to CD3-128. In some embodiments, a MBM of the disclosure comprises a CD3 ABM which comprises the VH and VL sequences of CD3-1. In some embodiments, a MBM of the disclosure comprises a CD3 ABM which comprises the VH and VL sequences of CD3-1. In some embodiments, a MBM of the disclosure comprises a CD3 ABM which comprises the VH and VL sequences of CD3-2. In some embodiments, a MBM of the disclosure comprises a CD3 ABM which comprises the VH and VL sequences of CD3-3. In some embodiments, a MBM of the disclosure comprises a CD3 ABM which comprises the VH and VL sequences of CD3-4. In some embodiments, a MBM of the disclosure comprises a CD3 ABM which comprises the VH and VL sequences of CD3-5. In some embodiments, a MBM of the disclosure comprises a CD3 ABM which comprises the VH and VL sequences of CD3-6. In some embodiments, a MBM of the disclosure comprises a CD3 ABM which comprises the VH and VL sequences of CD3-7. In some embodiments, a MBM of the disclosure comprises a CD3 ABM which comprises the VH and VL sequences of CD3-8. In some embodiments, a MBM of the disclosure comprises a CD3 ABM which comprises the VH and VL sequences of CD3-9. In some embodiments, a MBM of the disclosure comprises a CD3 ABM which comprises the VH and VL sequences of CD3-10. In some embodiments, a MBM of the disclosure comprises a CD3 ABM which comprises the VH and VL sequences of CD3-11. In some embodiments, a MBM of the disclosure comprises a CD3 ABM which comprises the VH and VL sequences of CD3-12. In some embodiments, a MBM of the disclosure comprises a CD3 ABM which comprises the VH and VL sequences of CD3-13. In some embodiments, a MBM of the disclosure comprises a CD3 ABM which comprises the VH and VL sequences of CD3-14. In some embodiments, a MBM of the disclosure comprises a CD3 ABM which comprises the VH and VL sequences of CD3-15. In some embodiments, a MBM of the disclosure comprises a CD3 ABM which comprises the VH and VL sequences of CD3-16. In some embodiments, a MBM of the disclosure comprises a CD3 ABM which comprises the VH and VL sequences of CD3-17. In some embodiments, a MBM of the disclosure comprises a CD3 ABM which comprises the VH and VL sequences of CD3-18. In some embodiments, a MBM of the disclosure comprises a CD3 ABM which comprises the VH and VL sequences of CD3-19. In some embodiments, a MBM of the disclosure comprises a CD3 ABM which comprises the VH and VL sequences of CD3-20. In some embodiments, a MBM of the disclosure comprises a CD3 ABM which comprises the VH and VL sequences of CD3-21. In some embodiments, a MBM of the disclosure comprises a CD3 ABM which comprises the VH and VL sequences of CD3-22. In some embodiments, a MBM of the disclosure comprises a CD3 ABM which comprises the VH and VL sequences of 003-23. In some embodiments, a MBM of the disclosure comprises a 003 ABM which comprises the VH and VL sequences of 003-24. In some embodiments, a MBM of the disclosure comprises a 003 ABM which comprises the VH and VL sequences of 003-25. In some embodiments, a MBM of the disclosure comprises a 003 ABM which comprises the VH and VL sequences of 003-26. In some embodiments, a MBM of the disclosure comprises a 003 ABM which comprises the VH and VL sequences of 003-27. In some embodiments, a MBM of the disclosure comprises a 003 ABM which comprises the VH and VL sequences of 003-28.

### 7.5.2. TCR-α/β ABMs

The MBMs (e.g., TBMs) of the disclosure can contain an ABM that specifically binds to the TCR-α chain, the TCR-β chain, or the TCR-αβ dimer. Exemplary anti-TCR-α/β antibodies are known in the art (see, e.g., US 2012/0034221; Borst et al., 1990, Hum Immunol. 29(3):175-88 (describing antibody BMA031)). The VH, VL, and Kabat C2R sequences of antibody BMA031 are provided in Table 9.

TABLE 9
BMA031 sequences

| | | SEQ |
|---|---|---|
| Domain | Sequence | ID NO: |
| BMA031 | KASGYKFTSYVMH | 240 |
| CDR-H1 | | |
| BMA031 | YINPYNDVTKYNEKFK | 241 |
| CDR-H2 | | |
| BMA031 | GSYYDYDGFVY | 242 |
| CDR-H3 | | |
| BMA031 | SATSSVSYMH | 243 |
| CDR-L1 | | |
| BMA031 | DTSKLAS | 149 |
| CDR-L2 | | |
| BMA031 | QQWSSNPLT | 178 |
| CDR-L3 | | |
| BMA031 | EVQLQQSGPELVKPGASVKMSCKASGYKFTSYVMH | 244 |
| VH | WVKQKPGQGLEWIGYINPYNDVTKYNEKFKGKATL | |
| | TSDKSSSTAYMELSSLTSEDSAVHYCARGSYYDYD | |
| | GFVYWGQGTLVTVSA | |
| BMA031 | QIVLTQSPAIMSASPGEKVTMTCSATSSVSYMHWY | 245 |
| VL | QQKSGTSPKRWIYDTSKLASGVPARFSGSGSGTSY | |
| | SLTISSMEAEDAATYYCQQWSSNPLTFGAGTKLEL | |
| | K | |

In an embodiment, a TCR ABM can comprise the CDR sequences of antibody BMA031. In other embodiments, a TCR ABM can comprise the VH and VL sequences of antibody BMA031.

### 7.5.3. TCR-γ/δ ABMs

The MBMs (e.g., TBMs) of the disclosure can contain an ABM that specifically binds to the TCR-γ chain, the TCR-δ chain, or the TCR-γδ dimer. Exemplary anti-TCR-γ/δ antibodies are known in the art (see, e.g., U.S. Pat. No. 5,980,892 (describing δTCS1, produced by the hybridoma deposited with the ATCC as accession number HB 9578)).

### 7.6. TAA 1 and TAA 2 ABMs

The MBMs (e.g., TBMs) of the disclosure comprise at least two ABMs that bind specifically to different tumor-associated antigens that are expressed on cancerous B cells (TAAs, with the first TAA referred to as TAA 1 and the second TAA referred to as TAA 2). In some cases, each TAA is a human TAA. The antigen may or may not be present on normal cells. In certain embodiments, the TAA is expressed or upregulated on cancerous B cells as compared to normal B cells. In other embodiments, the TAA is a B cell lineage marker. Each TAA may be expressed on the same cancerous B cell or may be expressed on different cancerous B cells.

It is anticipated that any type of B cell malignancy may be targeted by the MBMs of the disclosure. Exemplary types of B cell malignancies that may be targeted include Hodgkin's lymphomas, non-Hodgkin's lymphomas (NHLs), and multiple myeloma. Examples of NHLs include diffuse large B-cell lymphoma (DLBCL), follicular lymphoma, chronic lymphocytic leukemia (CLL)/small lymphocytic lymphoma (SLL), mantle cell lymphoma (MCL), marginal zone lymphomas, Burkitt lymphoma, lymphoplasmacytic lymphoma (Waldenstrom macroglobulinemia), hairy cell leukemia, primary central nervous system (CNS) lymphoma, primary mediastinal large B-cell lymphoma, mediastinal grey-zone lymphoma (MGZL), splenic marginal zone B-cell lymphoma, extranodal marginal zone B-cell lymphoma of MALT, nodal marginal zone B-cell lymphoma, and primary effusion lymphoma.

Examples of TAAs that can be targeted by the MBMs (e.g., TBMs) of the disclosure include CD19, CD20, CD22, CD123, BCMA, CD33, CLL1, CD138 (also known as Syndecan-1, SDC1), CS1, CD38, CD133, FLT3, CD52, TNFRSF13C (TNF Receptor Superfamily Member 13C, also referred to in the art as BAFFR: B-Cell-Activating Factor Receptor), TNFRSF13B (TNF Receptor Superfamily Member 13B, also referred to in the art as TACI: Transmembrane Activator And CAML Interactor), CXCR4 (C—X—C Motif Chemokine Receptor 4), PD-L1 (programmed death-ligand 1), LY9 (lymphocyte antigen 9, also referred to in the art as CD229), CD200, FCGR2B (Fc fragment of IgG receptor IIb, also referred to in the art as CD32b), CD21, CD23, CD24, CD40L, CD72, CD79a, and CD79b.

In some embodiments of the disclosure, TAA 1 is CD19 and TAA 2 is CD20 (or vice versa).

In some embodiments of the disclosure, TAA 1 is CD19 and TAA 2 is CD22 (or vice versa).

In some embodiments of the disclosure, TAA 1 is CD19 and TAA 2 is CD123 (or vice versa).

In some embodiments of the disclosure, TAA 1 is CD19 and TAA 2 is BCMA (or vice versa).

In some embodiments of the disclosure, TAA 1 is CD19 and TAA 2 is CD33 (or vice versa).

In some embodiments of the disclosure, TAA 1 is CD19 and TAA 2 is CLL1 (or vice versa).

In some embodiments of the disclosure, TAA 1 is CD19 and TAA 2 is CD138 (or vice versa).

In some embodiments of the disclosure, TAA 1 is CD19 and TAA 2 is CS1 (or vice versa).

In some embodiments of the disclosure, TAA 1 is CD19 and TAA 2 is CD38 (or vice versa).

In some embodiments of the disclosure, TAA 1 is CD19 and TAA 2 is CD133 (or vice versa).

In some embodiments of the disclosure, TAA 1 is CD19 and TAA 2 is FLT3 (or vice versa).

In some embodiments of the disclosure, TAA 1 is CD19 and TAA 2 is CD52 (or vice versa).

In some embodiments of the disclosure, TAA 1 is CD19 and TAA 2 is TNFRSF13C (or vice versa).

In some embodiments of the disclosure, TAA 1 is CD19 and TAA 2 is TNFRSF13B (or vice versa).

In some embodiments of the disclosure, TAA 1 is CD19 and TAA 2 is CXCR4 (or vice versa).

In some embodiments of the disclosure, TAA 1 is CD19 and TAA 2 is PD-L1 (or vice versa).

In some embodiments of the disclosure, TAA 1 is CD19 and TAA 2 is LY9 (or vice versa).

In some embodiments of the disclosure, TAA 1 is CD19 and TAA 2 is CD200 (or vice versa).

In some embodiments of the disclosure, TAA 1 is CD19 and TAA 2 is FCGR2B (or vice versa).

In some embodiments of the disclosure, TAA 1 is CD19 and TAA 2 is CD21 (or vice versa).

In some embodiments of the disclosure, TAA 1 is CD19 and TAA 2 is CD23 (or vice versa).

In some embodiments of the disclosure, TAA 1 is CD19 and TAA 2 is CD24 (or vice versa).

In some embodiments of the disclosure, TAA 1 is CD19 and TAA 2 is CD40L (or vice versa).

In some embodiments of the disclosure, TAA 1 is CD19 and TAA 2 is CD72 (or vice versa).

In some embodiments of the disclosure, TAA 1 is CD19 and TAA 2 is CD79a (or vice versa).

In some embodiments of the disclosure, TAA 1 is CD19 and TAA 2 is CD79b (or vice versa).

In some embodiments of the disclosure, TAA 1 is CD20 and TAA 2 is CD22 (or vice versa).

In some embodiments of the disclosure, TAA 1 is CD20 and TAA 2 is CD123 (or vice versa).

In some embodiments of the disclosure, TAA 1 is CD20 and TAA 2 is BCMA (or vice versa).

In some embodiments of the disclosure, TAA 1 is CD20 and TAA 2 is CD33 (or vice versa).

In some embodiments of the disclosure, TAA 1 is CD20 and TAA 2 is CLL1 (or vice versa).

In some embodiments of the disclosure, TAA 1 is CD20 and TAA 2 is CD138 (or vice versa).

In some embodiments of the disclosure, TAA 1 is CD20 and TAA 2 is CS1 (or vice versa).

In some embodiments of the disclosure, TAA 1 is CD20 and TAA 2 is CD38 (or vice versa).

In some embodiments of the disclosure, TAA 1 is CD20 and TAA 2 is CD133 (or vice versa).

In some embodiments of the disclosure, TAA 1 is CD20 and TAA 2 is FLT3 (or vice versa).

In some embodiments of the disclosure, TAA 1 is CD20 and TAA 2 is CD52 (or vice versa).

In some embodiments of the disclosure, TAA 1 is CD20 and TAA 2 is TNFRSF13C (or vice versa).

In some embodiments of the disclosure, TAA 1 is CD20 and TAA 2 is TNFRSF13B (or vice versa).

In some embodiments of the disclosure, TAA 1 is CD20 and TAA 2 is CXCR4 (or vice versa).

In some embodiments of the disclosure, TAA 1 is CD20 and TAA 2 is PD-L1 (or vice versa).

In some embodiments of the disclosure, TAA 1 is CD20 and TAA 2 is LY9 (or vice versa).

In some embodiments of the disclosure, TAA 1 is CD20 and TAA 2 is CD200 (or vice versa).

In some embodiments of the disclosure, TAA 1 is CD20 and TAA 2 is FCGR2B (or vice versa).

In some embodiments of the disclosure, TAA 1 is CD20 and TAA 2 is CD21 (or vice versa).

In some embodiments of the disclosure, TAA 1 is CD20 and TAA 2 is CD23 (or vice versa).

In some embodiments of the disclosure, TAA 1 is CD20 and TAA 2 is CD24 (or vice versa).

In some embodiments of the disclosure, TAA 1 is CD20 and TAA 2 is CD40L (or vice versa).

In some embodiments of the disclosure, TAA 1 is CD20 and TAA 2 is CD72 (or vice versa).

In some embodiments of the disclosure, TAA 1 is CD20 and TAA 2 is CD79a (or vice versa).

In some embodiments of the disclosure, TAA 1 is CD20 and TAA 2 is CD79b (or vice versa).

In some embodiments of the disclosure, TAA 1 is CD22 and TAA 2 is CD123 (or vice versa).

In some embodiments of the disclosure, TAA 1 is CD22 and TAA 2 is BCMA (or vice versa).

In some embodiments of the disclosure, TAA 1 is CD22 and TAA 2 is CD33 (or vice versa).

In some embodiments of the disclosure, TAA 1 is CD22 and TAA 2 is CLL1 (or vice versa).

In some embodiments of the disclosure, TAA 1 is CD22 and TAA 2 is CD138 (or vice versa).

In some embodiments of the disclosure, TAA 1 is CD22 and TAA 2 is CS1 (or vice versa).

In some embodiments of the disclosure, TAA 1 is CD22 and TAA 2 is CD38 (or vice versa).

In some embodiments of the disclosure, TAA 1 is CD22 and TAA 2 is CD133 (or vice versa).

In some embodiments of the disclosure, TAA 1 is CD22 and TAA 2 is FLT3 (or vice versa).

In some embodiments of the disclosure, TAA 1 is CD22 and TAA 2 is CD52 (or vice versa).

In some embodiments of the disclosure, TAA 1 is CD22 and TAA 2 is TNFRSF13C (or vice versa).

In some embodiments of the disclosure, TAA 1 is CD22 and TAA 2 is TNFRSF13B (or vice versa).

In some embodiments of the disclosure, TAA 1 is CD22 and TAA 2 is CXCR4 (or vice versa).

In some embodiments of the disclosure, TAA 1 is CD22 and TAA 2 is PD-L1 (or vice versa).

In some embodiments of the disclosure, TAA 1 is CD22 and TAA 2 is LY9 (or vice versa).

In some embodiments of the disclosure, TAA 1 is CD22 and TAA 2 is CD200 (or vice versa).

In some embodiments of the disclosure, TAA 1 is CD22 and TAA 2 is FCGR2B (or vice versa).

In some embodiments of the disclosure, TAA 1 is CD22 and TAA 2 is CD21 (or vice versa).

In some embodiments of the disclosure, TAA 1 is CD22 and TAA 2 is CD23 (or vice versa).

In some embodiments of the disclosure, TAA 1 is CD22 and TAA 2 is CD24 (or vice versa).

In some embodiments of the disclosure, TAA 1 is CD22 and TAA 2 is CD40L (or vice versa).

In some embodiments of the disclosure, TAA 1 is CD22 and TAA 2 is CD72 (or vice versa).

In some embodiments of the disclosure, TAA 1 is CD22 and TAA 2 is CD79a (or vice versa).

In some embodiments of the disclosure, TAA 1 is CD22 and TAA 2 is CD79b (or vice versa).

In some embodiments of the disclosure, TAA 1 is CD123 and TAA 2 is BCMA (or vice versa).

In some embodiments of the disclosure, TAA 1 is CD123 and TAA 2 is CD33 (or vice versa).

In some embodiments of the disclosure, TAA 1 is CD123 and TAA 2 is CLL1 (or vice versa).

In some embodiments of the disclosure, TAA 1 is CD123 and TAA 2 is CD138 (or vice versa).

In some embodiments of the disclosure, TAA 1 is CD123 and TAA 2 is CS1 (or vice versa).

In some embodiments of the disclosure, TAA 1 is CD123 and TAA 2 is CD38 (or vice versa).

In some embodiments of the disclosure, TAA 1 is CD123 and TAA 2 is CD133 (or vice versa).

In some embodiments of the disclosure, TAA 1 is CD123 and TAA 2 is FLT3 (or vice versa).

In some embodiments of the disclosure, TAA 1 is CD123 and TAA 2 is CD52 (or vice versa).

In some embodiments of the disclosure, TAA 1 is CD123 and TAA 2 is TNFRSF13C (or vice versa).

In some embodiments of the disclosure, TAA 1 is CD123 and TAA 2 is TNFRSF13B (or vice versa).

In some embodiments of the disclosure, TAA 1 is CD123 and TAA 2 is CXCR4 (or vice versa).

In some embodiments of the disclosure, TAA 1 is CD123 and TAA 2 is PD-L1 (or vice versa).

In some embodiments of the disclosure, TAA 1 is CD123 and TAA 2 is LY9 (or vice versa).

In some embodiments of the disclosure, TAA 1 is CD123 and TAA 2 is CD200 (or vice versa).

In some embodiments of the disclosure, TAA 1 is CD123 and TAA 2 is FCGR2B (or vice versa).

In some embodiments of the disclosure, TAA 1 is CD123 and TAA 2 is CD21 (or vice versa).

In some embodiments of the disclosure, TAA 1 is CD123 and TAA 2 is CD23 (or vice versa).

In some embodiments of the disclosure, TAA 1 is CD123 and TAA 2 is CD24 (or vice versa).

In some embodiments of the disclosure, TAA 1 is CD123 and TAA 2 is CD40L (or vice versa).

In some embodiments of the disclosure, TAA 1 is CD123 and TAA 2 is CD72 (or vice versa).

In some embodiments of the disclosure, TAA 1 is CD123 and TAA 2 is CD79a (or vice versa).

In some embodiments of the disclosure, TAA 1 is CD123 and TAA 2 is CD79b (or vice versa).

In some embodiments of the disclosure, TAA 1 is BCMA and TAA 2 is CD33 (or vice versa).

In some embodiments of the disclosure, TAA 1 is BCMA and TAA 2 is CLL1 (or vice versa).

In some embodiments of the disclosure, TAA 1 is BCMA and TAA 2 is CD138 (or vice versa).

In some embodiments of the disclosure, TAA 1 is BCMA and TAA 2 is CS1 (or vice versa).

In some embodiments of the disclosure, TAA 1 is BCMA and TAA 2 is CD38 (or vice versa).

In some embodiments of the disclosure, TAA 1 is BCMA and TAA 2 is CD133 (or vice versa).

In some embodiments of the disclosure, TAA 1 is BCMA and TAA 2 is FLT3 (or vice versa).

In some embodiments of the disclosure, TAA 1 is BCMA and TAA 2 is CD52 (or vice versa).

In some embodiments of the disclosure, TAA 1 is BCMA and TAA 2 is TNFRSF13C (or vice versa).

In some embodiments of the disclosure, TAA 1 is BCMA and TAA 2 is TNFRSF13B (or vice versa).

In some embodiments of the disclosure, TAA 1 is BCMA and TAA 2 is CXCR4 (or vice versa).

In some embodiments of the disclosure, TAA 1 is BCMA and TAA 2 is PD-L1 (or vice versa).

In some embodiments of the disclosure, TAA 1 is BCMA and TAA 2 is LY9 (or vice versa).

In some embodiments of the disclosure, TAA 1 is BCMA and TAA 2 is CD200 (or vice versa).

In some embodiments of the disclosure, TAA 1 is BCMA and TAA 2 is FCGR2B (or vice versa).

In some embodiments of the disclosure, TAA 1 is BCMA and TAA 2 is CD21 (or vice versa).

In some embodiments of the disclosure, TAA 1 is BCMA and TAA 2 is CD23 (or vice versa).

In some embodiments of the disclosure, TAA 1 is BCMA and TAA 2 is CD24 (or vice versa).

In some embodiments of the disclosure, TAA 1 is BCMA and TAA 2 is CD40L (or vice versa).

In some embodiments of the disclosure, TAA 1 is BCMA and TAA 2 is CD72 (or vice versa).

In some embodiments of the disclosure, TAA 1 is BCMA and TAA 2 is CD79a (or vice versa).

In some embodiments of the disclosure, TAA 1 is BCMA and TAA 2 is CD79b (or vice versa).

In some embodiments of the disclosure, TAA 1 is CD33 and TAA 2 is CLL1 (or vice versa).

In some embodiments of the disclosure, TAA 1 is CD33 and TAA 2 is CD138 (or vice versa).

In some embodiments of the disclosure, TAA 1 is CD33 and TAA 2 is CS1 (or vice versa).

In some embodiments of the disclosure, TAA 1 is CD33 and TAA 2 is CD38 (or vice versa).

In some embodiments of the disclosure, TAA 1 is CD33 and TAA 2 is CD133 (or vice versa).

In some embodiments of the disclosure, TAA 1 is CD33 and TAA 2 is FLT3 (or vice versa).

In some embodiments of the disclosure, TAA 1 is CD33 and TAA 2 is CD52 (or vice versa).

In some embodiments of the disclosure, TAA 1 is CD33 and TAA 2 is TNFRSF13C (or vice versa).

In some embodiments of the disclosure, TAA 1 is CD33 and TAA 2 is TNFRSF13B (or vice versa).

In some embodiments of the disclosure, TAA 1 is CD33 and TAA 2 is CXCR4 (or vice versa).

In some embodiments of the disclosure, TAA 1 is CD33 and TAA 2 is PD-L1 (or vice versa).

In some embodiments of the disclosure, TAA 1 is CD33 and TAA 2 is LY9 (or vice versa).

In some embodiments of the disclosure, TAA 1 is CD33 and TAA 2 is CD200 (or vice versa).

In some embodiments of the disclosure, TAA 1 is CD33 and TAA 2 is FCGR2B (or vice versa).

In some embodiments of the disclosure, TAA 1 is CD33 and TAA 2 is CD21 (or vice versa).

In some embodiments of the disclosure, TAA 1 is CD33 and TAA 2 is CD23 (or vice versa).

In some embodiments of the disclosure, TAA 1 is CD33 and TAA 2 is CD24 (or vice versa).

In some embodiments of the disclosure, TAA 1 is CD33 and TAA 2 is CD40L (or vice versa).

In some embodiments of the disclosure, TAA 1 is CD33 and TAA 2 is CD72 (or vice versa).

In some embodiments of the disclosure, TAA 1 is CD33 and TAA 2 is CD79a (or vice versa).

In some embodiments of the disclosure, TAA 1 is CD33 and TAA 2 is CD79b (or vice versa).

In some embodiments of the disclosure, TAA 1 is CLL1 and TAA 2 is CD138 (or vice versa).

In some embodiments of the disclosure, TAA 1 is CLL1 and TAA 2 is CS1 (or vice versa).

In some embodiments of the disclosure, TAA 1 is CLL1 and TAA 2 is CD38 (or vice versa).

In some embodiments of the disclosure, TAA 1 is CLL1 and TAA 2 is CD133 (or vice versa).

In some embodiments of the disclosure, TAA 1 is CLL1 and TAA 2 is FLT3 (or vice versa).

In some embodiments of the disclosure, TAA 1 is CLL1 and TAA 2 is CD52 (or vice versa).

In some embodiments of the disclosure, TAA 1 is CLL1 and TAA 2 is TNFRSF13C (or vice versa).

In some embodiments of the disclosure, TAA 1 is CLL1 and TAA 2 is TNFRSF13B (or vice versa).

In some embodiments of the disclosure, TAA 1 is CLL1 and TAA 2 is CXCR4 (or vice versa).

In some embodiments of the disclosure, TAA 1 is CLL1 and TAA 2 is PD-L1 (or vice versa).

In some embodiments of the disclosure, TAA 1 is CLL1 and TAA 2 is LY9 (or vice versa).

In some embodiments of the disclosure, TAA 1 is CLL1 and TAA 2 is CD200 (or vice versa).

In some embodiments of the disclosure, TAA 1 is CLL1 and TAA 2 is FCGR2B (or vice versa).

In some embodiments of the disclosure, TAA 1 is CLL1 and TAA 2 is CD21 (or vice versa).

In some embodiments of the disclosure, TAA 1 is CLL1 and TAA 2 is CD23 (or vice versa).

In some embodiments of the disclosure, TAA 1 is CLL1 and TAA 2 is CD24 (or vice versa).

In some embodiments of the disclosure, TAA 1 is CLL1 and TAA 2 is CD40L (or vice versa).

In some embodiments of the disclosure, TAA 1 is CLL1 and TAA 2 is CD72 (or vice versa).

In some embodiments of the disclosure, TAA 1 is CLL1 and TAA 2 is CD79a (or vice versa).

In some embodiments of the disclosure, TAA 1 is CLL1 and TAA 2 is CD79b (or vice versa).

In some embodiments of the disclosure, TAA 1 is CD138 and TAA 2 is CS1 (or vice versa).

In some embodiments of the disclosure, TAA 1 is CD138 and TAA 2 is CD38 (or vice versa).

In some embodiments of the disclosure, TAA 1 is CD138 and TAA 2 is CD133 (or vice versa).

In some embodiments of the disclosure, TAA 1 is CD138 and TAA 2 is FLT3 (or vice versa).

In some embodiments of the disclosure, TAA 1 is CD138 and TAA 2 is CD52 (or vice versa).

In some embodiments of the disclosure, TAA 1 is CD138 and TAA 2 is TNFRSF13C (or vice versa).

In some embodiments of the disclosure, TAA 1 is CD138 and TAA 2 is TNFRSF13B (or vice versa).

In some embodiments of the disclosure, TAA 1 is CD138 and TAA 2 is CXCR4 (or vice versa).

In some embodiments of the disclosure, TAA 1 is CD138 and TAA 2 is PD-L1 (or vice versa).

In some embodiments of the disclosure, TAA 1 is CD138 and TAA 2 is LY9 (or vice versa).

In some embodiments of the disclosure, TAA 1 is CD138 and TAA 2 is CD200 (or vice versa).

In some embodiments of the disclosure, TAA 1 is CD138 and TAA 2 is FCGR2B (or vice versa).

In some embodiments of the disclosure, TAA 1 is CD138 and TAA 2 is CD21 (or vice versa).

In some embodiments of the disclosure, TAA 1 is CD138 and TAA 2 is CD23 (or vice versa).

In some embodiments of the disclosure, TAA 1 is CD138 and TAA 2 is CD24 (or vice versa).

In some embodiments of the disclosure, TAA 1 is CD138 and TAA 2 is CD40L (or vice versa).

In some embodiments of the disclosure, TAA 1 is CD138 and TAA 2 is CD72 (or vice versa).

In some embodiments of the disclosure, TAA 1 is CD138 and TAA 2 is CD79a (or vice versa).

In some embodiments of the disclosure, TAA 1 is CD138 and TAA 2 is CD79b (or vice versa).

In some embodiments of the disclosure, TAA 1 is CS1 and TAA 2 is CD38 (or vice versa).

In some embodiments of the disclosure, TAA 1 is CS1 and TAA 2 is CD133 (or vice versa).

In some embodiments of the disclosure, TAA 1 is CS1 and TAA 2 is FLT3 (or vice versa).

In some embodiments of the disclosure, TAA 1 is CS1 and TAA 2 is CD52 (or vice versa).

In some embodiments of the disclosure, TAA 1 is CS1 and TAA 2 is TNFRSF13C (or vice versa).

In some embodiments of the disclosure, TAA 1 is CS1 and TAA 2 is TNFRSF13B (or vice versa).

In some embodiments of the disclosure, TAA 1 is CS1 and TAA 2 is CXCR4 (or vice versa).

In some embodiments of the disclosure, TAA 1 is CS1 and TAA 2 is PD-L1 (or vice versa).

In some embodiments of the disclosure, TAA 1 is CS1 and TAA 2 is LY9 (or vice versa).

In some embodiments of the disclosure, TAA 1 is CS1 and TAA 2 is CD200 (or vice versa).

In some embodiments of the disclosure, TAA 1 is CS1 and TAA 2 is FCGR2B (or vice versa).

In some embodiments of the disclosure, TAA 1 is CS1 and TAA 2 is CD21 (or vice versa).

In some embodiments of the disclosure, TAA 1 is CS1 and TAA 2 is CD23 (or vice versa).

In some embodiments of the disclosure, TAA 1 is CS1 and TAA 2 is CD24 (or vice versa).

In some embodiments of the disclosure, TAA 1 is CS1 and TAA 2 is CD40L (or vice versa).

In some embodiments of the disclosure, TAA 1 is CS1 and TAA 2 is CD72 (or vice versa).

In some embodiments of the disclosure, TAA 1 is CS1 and TAA 2 is CD79a (or vice versa).

In some embodiments of the disclosure, TAA 1 is CS1 and TAA 2 is CD79b (or vice versa).

In some embodiments of the disclosure, TAA 1 is CD38 and TAA 2 is CD133 (or vice versa).

In some embodiments of the disclosure, TAA 1 is CD38 and TAA 2 is FLT3 (or vice versa).

In some embodiments of the disclosure, TAA 1 is CD38 and TAA 2 is CD52 (or vice versa).

In some embodiments of the disclosure, TAA 1 is CD38 and TAA 2 is TNFRSF13C (or vice versa).

In some embodiments of the disclosure, TAA 1 is CD38 and TAA 2 is TNFRSF13B (or vice versa).

In some embodiments of the disclosure, TAA 1 is CD38 and TAA 2 is CXCR4 (or vice versa).

In some embodiments of the disclosure, TAA 1 is CD38 and TAA 2 is PD-L1 (or vice versa).

In some embodiments of the disclosure, TAA 1 is CD38 and TAA 2 is LY9 (or vice versa).

In some embodiments of the disclosure, TAA 1 is CD38 and TAA 2 is CD200 (or vice versa).

In some embodiments of the disclosure, TAA 1 is CD38 and TAA 2 is FCGR2B (or vice versa).

In some embodiments of the disclosure, TAA 1 is CD38 and TAA 2 is CD21 (or vice versa).

In some embodiments of the disclosure, TAA 1 is CD38 and TAA 2 is CD23 (or vice versa).

In some embodiments of the disclosure, TAA 1 is CD38 and TAA 2 is CD24 (or vice versa).

In some embodiments of the disclosure, TAA 1 is CD38 and TAA 2 is CD40L (or vice versa).

In some embodiments of the disclosure, TAA 1 is CD38 and TAA 2 is CD72 (or vice versa).

In some embodiments of the disclosure, TAA 1 is CD38 and TAA 2 is CD79a (or vice versa).

In some embodiments of the disclosure, TAA 1 is CD38 and TAA 2 is CD79b (or vice versa).

In some embodiments of the disclosure, TAA 1 is CD133 and TAA 2 is FLT3 (or vice versa).

In some embodiments of the disclosure, TAA 1 is CD133 and TAA 2 is CD52 (or vice versa).

In some embodiments of the disclosure, TAA 1 is CD133 and TAA 2 is TNFRSF13C (or vice versa).

In some embodiments of the disclosure, TAA 1 is CD133 and TAA 2 is TNFRSF13B (or vice versa).

In some embodiments of the disclosure, TAA 1 is CD133 and TAA 2 is CXCR4 (or vice versa).

In some embodiments of the disclosure, TAA 1 is CD133 and TAA 2 is PD-L1 (or vice versa).

In some embodiments of the disclosure, TAA 1 is CD133 and TAA 2 is LY9 (or vice versa).

In some embodiments of the disclosure, TAA 1 is CD133 and TAA 2 is CD200 (or vice versa).

In some embodiments of the disclosure, TAA 1 is CD133 and TAA 2 is FCGR2B (or vice versa).

In some embodiments of the disclosure, TAA 1 is CD133 and TAA 2 is CD21 (or vice versa).

In some embodiments of the disclosure, TAA 1 is CD133 and TAA 2 is CD23 (or vice versa).

In some embodiments of the disclosure, TAA 1 is CD133 and TAA 2 is CD24 (or vice versa).

In some embodiments of the disclosure, TAA 1 is CD133 and TAA 2 is CD40L (or vice versa).

In some embodiments of the disclosure, TAA 1 is CD133 and TAA 2 is CD72 (or vice versa).

In some embodiments of the disclosure, TAA 1 is CD133 and TAA 2 is CD79a (or vice versa).

In some embodiments of the disclosure, TAA 1 is CD133 and TAA 2 is CD79b (or vice versa).

In some embodiments of the disclosure, TAA 1 is FLT3 and TAA 2 is CD52 (or vice versa).

In some embodiments of the disclosure, TAA 1 is FLT3 and TAA 2 is TNFRSF13C (or vice versa).

In some embodiments of the disclosure, TAA 1 is FLT3 and TAA 2 is TNFRSF13B (or vice versa).

In some embodiments of the disclosure, TAA 1 is FLT3 and TAA 2 is CXCR4 (or vice versa).

In some embodiments of the disclosure, TAA 1 is FLT3 and TAA 2 is PD-L1 (or vice versa).

In some embodiments of the disclosure, TAA 1 is FLT3 and TAA 2 is LY9 (or vice versa).

In some embodiments of the disclosure, TAA 1 is FLT3 and TAA 2 is CD200 (or vice versa).

In some embodiments of the disclosure, TAA 1 is FLT3 and TAA 2 is FCGR2B (or vice versa).

In some embodiments of the disclosure, TAA 1 is FLT3 and TAA 2 is CD21 (or vice versa).

In some embodiments of the disclosure, TAA 1 is FLT3 and TAA 2 is CD23 (or vice versa).

In some embodiments of the disclosure, TAA 1 is FLT3 and TAA 2 is CD24 (or vice versa).

In some embodiments of the disclosure, TAA 1 is FLT3 and TAA 2 is CD40L (or vice versa).

In some embodiments of the disclosure, TAA 1 is FLT3 and TAA 2 is CD72 (or vice versa).

In some embodiments of the disclosure, TAA 1 is FLT3 and TAA 2 is CD79a (or vice versa).

In some embodiments of the disclosure, TAA 1 is FLT3 and TAA 2 is CD79b (or vice versa).

In some embodiments of the disclosure, TAA 1 is CD52 and TAA 2 is TNFRSF13C (or vice versa).

In some embodiments of the disclosure, TAA 1 is CD52 and TAA 2 is TNFRSF13B (or vice versa).

In some embodiments of the disclosure, TAA 1 is CD52 and TAA 2 is CXCR4 (or vice versa).

In some embodiments of the disclosure, TAA 1 is CD52 and TAA 2 is PD-L1 (or vice versa).

In some embodiments of the disclosure, TAA 1 is CD52 and TAA 2 is LY9 (or vice versa).

In some embodiments of the disclosure, TAA 1 is CD52 and TAA 2 is CD200 (or vice versa).

In some embodiments of the disclosure, TAA 1 is CD52 and TAA 2 is FCGR2B (or vice versa).

In some embodiments of the disclosure, TAA 1 is CD52 and TAA 2 is CD21 (or vice versa).

In some embodiments of the disclosure, TAA 1 is CD52 and TAA 2 is CD23 (or vice versa).

In some embodiments of the disclosure, TAA 1 is CD52 and TAA 2 is CD24 (or vice versa).

In some embodiments of the disclosure, TAA 1 is CD52 and TAA 2 is CD40L (or vice versa).

In some embodiments of the disclosure, TAA 1 is CD52 and TAA 2 is CD72 (or vice versa).

In some embodiments of the disclosure, TAA 1 is CD52 and TAA 2 is CD79a (or vice versa).

In some embodiments of the disclosure, TAA 1 is CD52 and TAA 2 is CD79b (or vice versa).

In some embodiments of the disclosure, TAA 1 is TNFRSF13C and TAA 2 is TNFRSF13B (or vice versa).

In some embodiments of the disclosure, TAA 1 is TNFRSF13C and TAA 2 is CXCR4 (or vice versa).

In some embodiments of the disclosure, TAA 1 is TNFRSF13C and TAA 2 is PD-L1 (or vice versa).

In some embodiments of the disclosure, TAA 1 is TNFRSF13C and TAA 2 is LY9 (or vice versa).

In some embodiments of the disclosure, TAA 1 is TNFRSF13C and TAA 2 is CD200 (or vice versa).

In some embodiments of the disclosure, TAA 1 is TNFRSF13C and TAA 2 is FCGR2B (or vice versa).

In some embodiments of the disclosure, TAA 1 is TNFRSF13C and TAA 2 is CD21 (or vice versa).

In some embodiments of the disclosure, TAA 1 is TNFRSF13C and TAA 2 is CD23 (or vice versa).

In some embodiments of the disclosure, TAA 1 is TNFRSF13C and TAA 2 is CD24 (or vice versa).

In some embodiments of the disclosure, TAA 1 is TNFRSF13C and TAA 2 is CD40L (or vice versa).

In some embodiments of the disclosure, TAA 1 is TNFRSF13C and TAA 2 is CD72 (or vice versa).

In some embodiments of the disclosure, TAA 1 is TNFRSF13C and TAA 2 is CD79a (or vice versa).

In some embodiments of the disclosure, TAA 1 is TNFRSF13C and TAA 2 is CD79b (or vice versa).

In some embodiments of the disclosure, TAA 1 is TNFRSF13B and TAA 2 is CXCR4 (or vice versa).

In some embodiments of the disclosure, TAA 1 is TNFRSF13B and TAA 2 is PD-L1 (or vice versa).

In some embodiments of the disclosure, TAA 1 is TNFRSF13B and TAA 2 is LY9 (or vice versa).

In some embodiments of the disclosure, TAA 1 is TNFRSF13B and TAA 2 is CD200 (or vice versa).

In some embodiments of the disclosure, TAA 1 is TNFRSF13B and TAA 2 is FCGR2B (or vice versa).

In some embodiments of the disclosure, TAA 1 is TNFRSF13B and TAA 2 is CD21 (or vice versa).

In some embodiments of the disclosure, TAA 1 is TNFRSF13B and TAA 2 is CD23 (or vice versa).

In some embodiments of the disclosure, TAA 1 is TNFRSF13B and TAA 2 is CD24 (or vice versa).

In some embodiments of the disclosure, TAA 1 is TNFRSF13B and TAA 2 is CD40L (or vice versa).

In some embodiments of the disclosure, TAA 1 is TNFRSF13B and TAA 2 is CD72 (or vice versa).

In some embodiments of the disclosure, TAA 1 is TNFRSF13B and TAA 2 is CD79a (or vice versa).

In some embodiments of the disclosure, TAA 1 is TNFRSF13B and TAA 2 is CD79b (or vice versa).

In some embodiments of the disclosure, TAA 1 is CXCR4 and TAA 2 is PD-L1 (or vice versa).

In some embodiments of the disclosure, TAA 1 is CXCR4 and TAA 2 is LY9 (or vice versa).

In some embodiments of the disclosure, TAA 1 is CXCR4 and TAA 2 is CD200 (or vice versa).

In some embodiments of the disclosure, TAA 1 is CXCR4 and TAA 2 is FCGR2B (or vice versa).

In some embodiments of the disclosure, TAA 1 is CXCR4 and TAA 2 is CD21 (or vice versa).

In some embodiments of the disclosure, TAA 1 is CXCR4 and TAA 2 is CD23 (or vice versa).

In some embodiments of the disclosure, TAA 1 is CXCR4 and TAA 2 is CD24 (or vice versa).

In some embodiments of the disclosure, TAA 1 is CXCR4 and TAA 2 is CD40L (or vice versa).

In some embodiments of the disclosure, TAA 1 is CXCR4 and TAA 2 is CD72 (or vice versa).

In some embodiments of the disclosure, TAA 1 is CXCR4 and TAA 2 is CD79a (or vice versa).

In some embodiments of the disclosure, TAA 1 is CXCR4 and TAA 2 is CD79b (or vice versa).

In some embodiments of the disclosure, TAA 1 is PD-L1 and TAA 2 is LY9 (or vice versa).

In some embodiments of the disclosure, TAA 1 is PD-L1 and TAA 2 is CD200 (or vice versa).

In some embodiments of the disclosure, TAA 1 is PD-L1 and TAA 2 is FCGR2B (or vice versa).

In some embodiments of the disclosure, TAA 1 is PD-L1 and TAA 2 is CD21 (or vice versa).

In some embodiments of the disclosure, TAA 1 is PD-L1 and TAA 2 is CD23 (or vice versa).

In some embodiments of the disclosure, TAA 1 is PD-L1 and TAA 2 is CD24 (or vice versa).

In some embodiments of the disclosure, TAA 1 is PD-L1 and TAA 2 is CD40L (or vice versa).

In some embodiments of the disclosure, TAA 1 is PD-L1 and TAA 2 is CD72 (or vice versa).

In some embodiments of the disclosure, TAA 1 is PD-L1 and TAA 2 is CD79a (or vice versa).

In some embodiments of the disclosure, TAA 1 is PD-L1 and TAA 2 is CD79b (or vice versa).

In some embodiments of the disclosure, TAA 1 is LY9 and TAA 2 is CD200 (or vice versa).

In some embodiments of the disclosure, TAA 1 is LY9 and TAA 2 is FCGR2B (or vice versa).

In some embodiments of the disclosure, TAA 1 is LY9 and TAA 2 is CD21 (or vice versa).

In some embodiments of the disclosure, TAA 1 is LY9 and TAA 2 is CD23 (or vice versa).

In some embodiments of the disclosure, TAA 1 is LY9 and TAA 2 is CD24 (or vice versa).

In some embodiments of the disclosure, TAA 1 is LY9 and TAA 2 is CD40L (or vice versa).

In some embodiments of the disclosure, TAA 1 is LY9 and TAA 2 is CD72 (or vice versa).

In some embodiments of the disclosure, TAA 1 is LY9 and TAA 2 is CD79a (or vice versa).

In some embodiments of the disclosure, TAA 1 is LY9 and TAA 2 is CD79b (or vice versa).

In some embodiments of the disclosure, TAA 1 is CD200 and TAA 2 is FCGR2B (or vice versa).

In some embodiments of the disclosure, TAA 1 is CD200 and TAA 2 is CD21 (or vice versa).

In some embodiments of the disclosure, TAA 1 is CD200 and TAA 2 is CD23 (or vice versa).

In some embodiments of the disclosure, TAA 1 is CD200 and TAA 2 is CD24 (or vice versa).

In some embodiments of the disclosure, TAA 1 is CD200 and TAA 2 is CD40L (or vice versa).

In some embodiments of the disclosure, TAA 1 is CD200 and TAA 2 is CD72 (or vice versa).

In some embodiments of the disclosure, TAA 1 is CD200 and TAA 2 is CD79a (or vice versa).

In some embodiments of the disclosure, TAA 1 is CD200 and TAA 2 is CD79b (or vice versa).

In some embodiments of the disclosure, TAA 1 is CD21 and TAA 2 is CD23 (or vice versa).

In some embodiments of the disclosure, TAA 1 is CD21 and TAA 2 is CD24 (or vice versa).

In some embodiments of the disclosure, TAA 1 is CD21 and TAA 2 is CD40L (or vice versa).

In some embodiments of the disclosure, TAA 1 is CD21 and TAA 2 is CD72 (or vice versa).

In some embodiments of the disclosure, TAA 1 is CD21 and TAA 2 is CD79a (or vice versa).

In some embodiments of the disclosure, TAA 1 is CD21 and TAA 2 is CD79b (or vice versa).

In some embodiments of the disclosure, TAA 1 is CD23 and TAA 2 is CD24 (or vice versa).

In some embodiments of the disclosure, TAA 1 is CD23 and TAA 2 is CD40L (or vice versa).

In some embodiments of the disclosure, TAA 1 is CD23 and TAA 2 is CD72 (or vice versa).

In some embodiments of the disclosure, TAA 1 is CD23 and TAA 2 is CD79a (or vice versa).

In some embodiments of the disclosure, TAA 1 is CD23 and TAA 2 is CD79b (or vice versa).

In some embodiments of the disclosure, TAA 1 is CD24 and TAA 2 is CD40L (or vice versa).

In some embodiments of the disclosure, TAA 1 is CD24 and TAA 2 is CD72 (or vice versa).

In some embodiments of the disclosure, TAA 1 is CD24 and TAA 2 is CD79a (or vice versa).

In some embodiments of the disclosure, TAA 1 is CD24 and TAA 2 is CD79b (or vice versa).

In some embodiments of the disclosure, TAA 1 is CD40L and TAA 2 is CD72 (or vice versa).

In some embodiments of the disclosure, TAA 1 is CD40L and TAA 2 is CD79a (or vice versa).

In some embodiments of the disclosure, TAA 1 is CD40L and TAA 2 is CD79b (or vice versa).

In some embodiments of the disclosure, TAA 1 is CD72 and TAA 2 is CD79a (or vice versa).

In some embodiments of the disclosure, TAA 1 is CD72 and TAA 2 is CD79b (or vice versa).

In some embodiments of the disclosure, TAA 1 is CD79a and TAA 2 is CD79b (or vice versa).

A TAA 2 ABM can comprise, for example, an anti-TAA antibody or an antigen-binding fragment thereof. The anti-TAA antibody or antigen-binding fragment can comprise, for example, the CDR sequences of an antibody set forth in Table 10. In some embodiments, the anti-TAA antibody or antigen-binding domain thereof has the heavy and light chain variable region sequences of an antibody set forth in Table 10.

TABLE 10
Exemplary Anti-Tumor-Associated Antigen Antibodies

| | Examples of Antibody Name |
|---|---|
| Target | and/or Reference(s) and/or Source |
| BCMA | Any BCMA antibody described in |
| | WO2012163805, WO200112812, |
| | or WO2003062401. |
| CD123 | Any CD123 antibody described in |
| | U.S. Pat. No. 8,852,551, EP2426148, |
| | WO 2014/138819, |
| | WO 2016/028896, |
| | or WO 2014/130635 |
| CD19 | Any CD19 antibody described in |
| | WO 2014/031687, |
| | WO 2012/079000, |
| | WO 2014/153270, or U.S. Pat. |
| | No. 7,741,465; the CD19 binder |
| | of Yescarta or Blinatumomab |
| CD20 | Rituximab, Ofatumumab, |
| | Ocrelizumab, Veltuzumab, |
| | or GA101 |
| CD22 | Any CD22 antibody described |
| | in Haso et al., 2013, Blood, |
| | 121(7): 1165-1174, |
| | Wayne et al., 2010, |
| | Clin Cancer Res 16(6): 1894-1903, |
| | Kato et al., 2013, Leuk Res |
| | 37(1):83-88, or Creative BioMart |
| | (creativebiomart.net): |
| | MOM-18047-S(P). |
| CD33 | Any CD33 antibody described |
| | in Bross et al., 2001, Clin Cancer |
| | Res 7(6):1490- 1496 (Gemtuzumab |
| | Ozogamicin, hP67.6), Caron et al., |
| | 1992, Cancer Res 52(24):6761-6767 |
| | (Lintuzumab, HuM195), Lapusan |
| | et al., 2012, Invest New Drugs |
| | 30(3):1121-1131 (AVE9633), |
| | Aigner et al., 2013, Leukemia 27(5): |
| | 1107-1115 (AMG330, CD33 BiTE), |
| | Dutour et al., 2012, Adv Hematol |
| | 2012:683065, or Pizzitola et al., |
| | 2014, Leukemia doi:10.1038/ |
| | Lue.2014.62. |
| CD38 | Daratumumab (see, e.g., Groen |
| | et al., 2010, Blood 116(21): |
| | 1261-1262; MOR202 (see, e.g., |
| | U.S. Pat. No. 8,263,746); or any |
| | CD38 antibody described in |
| | U.S. Pat. No. 8,362,211. |
| CLL-1 | PE-CLL1-hu Cat# 353604 |
| | (BioLegend); |
| | PE-CLL1 (CLEC12A) |
| | Cat# 562566 (BD); Any CLL-1 |
| | antibody described in WO |
| | 2014/051433 A1, US 2016/0368994 |
| | A1, US 2013/0295118 A1, |
| | U.S. Pat. No. 8,536,310 B2, |
| | Lu et al., 2014, Angewandte |
| | Chemie International Edition |
| | 53(37):9841-9845, or Leong |
| | et al., 2017, Blood 129(5):609-618 |
| CS1 | Elotuzumab (BMS), see e.g., |
| | Tai et al., 2008, Blood 112(4): |
| | 1329-37; Tai et al., 2007, |
| | Blood. 110(5): 1656-63. |
| FLT3 | Any FLT3 antibody described |
| | in WO 2011/076922, U.S. |
| | Pat. No. 5,777,084, EP0754230, |
| | or US 2009/0297529. |
| CD133 | Any CD133 antibody described |
| | in U.S. Pat No. 9,624,303, WO |
| | 2016/154623, or WO 2011/089211; |
| | 5E3 (ThermoFisher); MAB11331 |
| | (R&D Systems); MAB4310 |
| | (Millipore Sigma) |
| CD138 | Any CD138 antibody described |
| | in WO/2009/080829, |
| | WO/2017/014679, or |
| | U.S. Pat. No. 9,289,509; nBT062 |
| | (Biotest AG); MI15, B-A38, |
| | SP152, DL-101 (ThermoFisher) |
| CD52 | alemtuzumab (Genzyme); |
| | ANT1034 (see, Holgate et al., |
| | 2015, PLOS ONE 10(9): |
| | e0138123; any CD52 antibody |
| | described in WO/2010/132659; |
| | any CD52 antibody described |
| | in U.S. Pat No. 9708407; any |
| | CD52 antibody described in |
| | WO/2010/132659 |
| TNFRSF13C | Any TNFRSF13C antibody |
| | described in WO 2010/007082, |
| | U.S. Pat. No. 9,382,326 |
| TNFRSF13B | Any TNFRSF13B antibody |
| | described in WO 2004/011611; |
| | LS-C89973 (Lifespan |
| | Biosciences, Inc.) M02952-1 |
| | (Boster Biological Technology); |
| | MAB1041, MAB1741, and |
| | MAB174 (R&D Systems) |
| CXCR4 | Any CXCR4 antibody |
| | described in U.S. Pat. Nos. |
| | 7,138,496, 8,329, 178, |
| | 8,450,464, 9,249,223, |
| | or 9,260,527 |
| PD-L1 | Any PD-L1 antibody described |
| | in US 2015/0203580, |
| | US 2017/0058033, |
| | US 2017/0204184, |
| | U.S. Pat. No. 8,741,295, |
| | U.S. Pat. No. 9,789,183, or |
| | U.S. Pat. No. 9,637,546 |
| LY9 | HLy9.25 (e.g., Lifespan |
| | Biosciences, Inc. cat. no. |
| | LS-C112605); MAB1898 |
| | (R&D Systems) |
| CD200 | Any CD200 antibody described |
| | in U.S. Pat. No. 7,887,798; |
| | ab23552 (Abcam); Ox104 |
| | (ThermoFisher) |
| FCGR2B | Any FCGR2B antibody |
| | described in U.S. Pat No. |
| | 8,802,089 or WO 2017/103895; |
| | ab45143 (Abcam); AT130-2 |
| | (ThermoFisher); 2E10 |
| | (Millipore Sigma) |
| CD21 | ab75985 (Abcam); ab9492 |
| | (Abcam); 2G9 (ThermoFisher); |
| | HB5 (ThermoFisher); |
| | MAB4909 (R&D Systems) |
| CD23 | Any CD23 antibody described |
| | in U.S. Pat. No. 7,008,623 |
| | or U.S. Pat. No. 6,011,138; |
| | lumiliximab (Biogen); |
| | ab16702 (Abcam); SP23 |
| | (ThermoFisher) |
| CD24 | Any CD24 antibody described |
| | in U.S. Pat. No. 8,614,301; |
| | SN3 (ThermoFisher); |
| | SN3b (ThermoFisher); |
| | 2Q1282 (Santa Cruz |
| | Biotechnology); 3H1143 |
| | (Santa Cruz Biotechnology); |
| | ALB9 (Santa Cruz Biotechnology); |
| | MAB5248 (R&D Systems) |
| CD40L | Any CD40L antibody described |
| | in U.S. Pat. No. 9,228,018 |
| | or US 2003/0099642; 24-31 |
| | (Biolegend); ab52750 (Abcam); |
| | ab47204 (Abcam); CDP7657 |
| | (UCB Pharma); 5c8 (Biogen) |
| CD72 | 3F3 (Biolegend); Bu40 |
| | (ThermoFisher); H-7 |
| | (Santa Cruz Biotechnology); |
| | H-96 (Santa Cruz Biotechnology); |
| | G-5 (Santa Cruz Biotechnology); |
| | ab92509 (Abcam) |
| CD79a | ab62650 (Abcam); ab79414 |
| | (Abcam); MAB69201 (R&D |
| | Systems); HM57 (Bio- Rad) |
| CD79b | Any CD79b antibody |
| | described in WO 2014/011521; |
| | ab130422 (Abcam); ab134147 |
| | (Abcam); polatuzumab |
| | (Genentech) |

In certain embodiments, TAA 1 and TAA 2 are selected from CD19, CD20 and BCMA. In other embodiments, TAA 1 and TAA 2 are selected from BCMA and CD19. Exemplary BCMA and CD19 binding sequences are set forth in Sections 7.6.1 and 7.6.2, infra.

### 7.6.1. BCMA

In certain aspects, the present disclosure provides a MBM (e.g., a TBM) in which TAA 1 or TAA 2 is BCMA. BCMA is a tumor necrosis family receptor (TNFR) member expressed on cells of the B-cell lineage. BCMA expression is the highest on terminally differentiated B cells that assume the long lived plasma cell fate, including plasma cells, plasmablasts and a subpopulation of activated B cells and memory B cells. BCMA is involved in mediating the survival of plasma cells for maintaining long-term humoral immunity. The expression of BCMA has been recently linked to a number of cancers, autoimmune disorders, and infectious diseases. Cancers with increased expression of BCMA include some hematological cancers, such as multiple myeloma, Hodgkin's and non-Hodgkin's lymphoma, various leukemias, and glioblastoma.

MBMs (e.g., TBMs) comprising a TAA 1 or TAA 2 ABM that binds to BCMA can comprise, for example, an anti-BCMA antibody or an antigen-binding domain thereof. The anti-BCMA antibody or antigen-binding domain thereof can comprise, for example, CDR, VH, VL, or scFV sequences set forth in Tables 11A-11G.

TABLE 11A
BCMA Binders-Variable domain and scFv sequences

| Antibody | Domain | Sequence | SEQ ID NO. |
|---|---|---|---|
| BCMA-1 | VH | EVQLVESGGGLVQPGGSLRLSCAVSGFALSNHGMSWVRRAPGKGLEWV | 246 |
| | | SGIVYSGSTYYAASVKGRFTISRDNSRNTLYLQMNSLRPEDTAIYYCS | |
| | | AHGGESDVWGQGTTVTVSS | |
| | VL | DIQLTQSPSSLSASVGDRVTITCRASQSISSYLNWYQQKPGKAPKLLI | 247 |
| | | YAASSLQSGVPSRFSGSGSGTDFTLTISSLQPEDFATYYCQQSYSTPY | |
| | | TFGQGTKVEIK | |
| | scFv | EVQLVESGGGLVQPGGSLRLSCAVSGFALSNHGMSWVRRAPGKGLEWV | 248 |
| | | SGIVYSGSTYYAASVKGRFTISRDNSRNTLYLQMNSLRPEDTAIYYCS | |
| | | AHGGESDVWGQGTTVTVSSASGGGGSGGRASGGGGSDIQLTQSPSSLS | |
| | | ASVGDRVTITCRASQSISSYLNWYQQKPGKAPKLLIYAASSLQSGVPS | |
| | | RFSGSGSGTDFTLTISSLQPEDFATYYCQQSYSTPYTFGQGTKVEIK | |
| BCMA-2 | VH | QVQLVESGGGLVQPGRSLRLSCAASGFTFSNYAMSWVRQAPGKGLGWV | 249 |
| | | SGISRSGENTYYADSVKGRFTISRDNSKNTLYLQMNSLRDEDTAVYYC | |
| | | ARSPAHYYGGMDVWGQGTTVTVSS | |
| | VL | DIVLTQSPGTLSLSPGERATLSCRASQSISSSFLAWYQQKPGQAPRLL | 250 |
| | | IYGASRRATGIPDRFSGSGSGTDFTLTISRLEPEDSAVYYCQQYHSSP | |
| | | SWTFGQGTKLEIK | |
| | scFv | QVQLVESGGGLVQPGRSLRLSCAASGFTFSNYAMSWVRQAPGKGLGWV | 251 |
| | | SGISRSGENTYYADSVKGRFTISRDNSKNTLYLQMNSLRDEDTAVYYC | |
| | | ARSPAHYYGGMDVWGQGTTVTVSSASGGGGSGGRASGGGGSDIVLTQS | |
| | | PGTLSLSPGERATLSCRASQSISSSFLAWYQQKPGQAPRLLIYGASRR | |
| | | ATGIPDRFSGSGSGTDFTLTISRLEPEDSAVYYCQQYHSSPSWTFGQG | |
| | | TKLEIK | |
| BCMA-3 | VH | QVQLVESGGGLVQPGRSLRLSCAASGFTFDDYAMHWVRQAPGKGLEWV | 252 |
| | | SGISWNSGSIGYADSVKGRFTISRDNAKNSLYLQMNSLRAEDTALYYC | |
| | | SVHSFLAYWGQGTLVTVSS | |
| | VL | DIVMTQTPLSLPVTPGEPASISCRSSQSLLHSNGYNYLDWYLQKPGQS | 253 |
| | | PQLLIYLGSNRASGVPDRFSGSGSGTDFTLKISRVEAEDVGVYYCMQA | |
| | | LQTPYTFGQGTKVEIK | |
| | scFv | QVQLVESGGGLVQPGRSLRLSCAASGFTFDDYAMHWVRQAPGKGLEWV | 254 |
| | | SGISWNSGSIGYADSVKGRFTISRDNAKNSLYLQMNSLRAEDTALYYC | |
| | | SVHSFLAYWGQGTLVTVSSASGGGGSGGRASGGGGSDIVMTQTPLSLP | |
| | | VTPGEPASISCRSSQSLLHSNGYNYLDWYLQKPGQSPQLLIYLGSNRA | |
| | | SGVPDRFSGSGSGTDFTLKISRVEAEDVGVYYCMQALQTPYTFGQGTK | |
| | | VEIK | |
| BCMA-4 | VH | EVQLLESGGGLVQPGGSLRLSCAVSGFALSNHGMSWVRRAPGKGLEWV | 255 |
| | | SGIVYSGSTYYAASVKGRFTISRDNSRNTLYLQMNSLRPEDTAIYYCS | |
| | | AHGGESDVWGQGTTVTVSS | |
| | VL | DIVMTQTPLSLSVTPGQPASISCKSSQSLLRNDGKTPLYWYLQKAGQP | 256 |
| | | PQLLIYEVSNRFSGVPDRFSGSGSGTDFTLKISRVEAEDVGAYYCMQN | |
| | | IQFPSFGGGTKLEIK | |
| | scFv | EVQLLESGGGLVQPGGSLRLSCAVSGFALSNHGMSWVRRAPGKGLEWV | 257 |
| | | SGIVYSGSTYYAASVKGRFTISRDNSRNTLYLQMNSLRPEDTAIYYCS | |
| | | AHGGESDVWGQGTTVTVSSASGGGGSGGRASGGGGSDIVMTQTPLSLS | |
| | | VTPGQPASISCKSSQSLLRNDGKTPLYWYLQKAGQPPQLLIYEVSNRF | |
| | | SGVPDRFSGSGSGTDFTLKISRVEAEDVGAYYCMQNIQFPSFGGGTKL | |
| | | EIK | |
| BCMA-5 | VH | QVQLVQSGAEVRKTGASVKVSCKASGYIFDNFGINWVRQAPGQGLEWM | 258 |
| | | GWINPKNNNTNYAQKFQGRVTITADESTNTAYMEVSSLRSEDTAVYYC | |
| | | ARGPYYYQSYMDVWGQGTMVTVSS | |
| | VL | DIVMTQTPLSLPVTPGEPASISCRSSQSLLHSNGYNYLNWYLQKPGQS | 259 |
| | | PQLLIYLGSKRASGVPDRFSGSGSGTDFTLHITRVGAEDVGVYYCMQA | |
| | | LQTPYTFGQGTKLEIK | |
| | scFv | QVQLVQSGAEVRKTGASVKVSCKASGYIFDNFGINWVRQAPGQGLEWM | 260 |
| | | GWINPKNNNTNYAQKFQGRVTITADESTNTAYMEVSSLRSEDTAVYYC | |
| | | ARGPYYYQSYMDVWGQGTMVTVSSASGGGGSGGRASGGGGSDIVMTQT | |
| | | PLSLPVTPGEPASISCRSSQSLLHSNGYNYLNWYLQKPGQSPQLLIYL | |
| | | GSKRASGVPDRFSGSGSGTDFTLHITRVGAEDVGVYYCMQALQTPYTF | |
| | | GQGTKLEIK | |
| BCMA-6 | VH | QVQLQESGGGLVQPGGSLRLSCAASGFTFSSDAMTWVRQAPGKGLEWV | 261 |
| | | SVISGSGGTTYYADSVKGRFTISRDNSKNTLYLQMNSLRAEDTAVYYC | |
| | | AKLDSSGYYYARGPRYWGQGTLVTVSS | |
| | VL | DIQLTQSPSSLSASVGDRVTITCRASQSISSYLNWYQQKPGKAPKLLI | 262 |
| | | YGASTLASGVPARFSGSGSGTHFTLTINSLQSEDSATYYCQQSYKRAS | |
| | | FGQGTKVEIK | |
| | scFv | QVQLQESGGGLVQPGGSLRLSCAASGFTFSSDAMTWVRQAPGKGLEWV | 263 |
| | | SVISGSGGTTYYADSVKGRFTISRDNSKNTLYLQMNSLRAEDTAVYYC | |
| | | AKLDSSGYYYARGPRYWGQGTLVTVSSASGGGGSGGRASGGGGSDIQL | |
| | | TQSPSSLSASVGDRVTITCRASQSISSYLNWYQQKPGKAPKLLIYGAS | |
| | | TLASGVPARFSGSGSGTHFTLTINSLQSEDSATYYCQQSYKRASFGQG | |
| | | TKVEIK | |
| BCMA-7 | VH | QVQLVQSGAEVKKPGASVKVSCKASGYTFSNYGITWVRQAPGQGLEWM | 264 |
| | | GWISAYNGNTNYAQKFQGRVTMTRNTSISTAYMELSSLRSEDTAVYYC | |
| | | ARGPYYYYMDVWGKGTMVTVSS | |
| | VL | EIVMTQSPLSLPVTPGEPASISCRSSQSLLYSNGYNYVDWYLQKPGQS | 265 |
| | | PQLLIYLGSNRASGVPDRFSGSGSGTDFKLQISRVEAEDVGIYYCMQG | |
| | | RQFPYSFGQGTKVEIK | |
| | scFv | QVQLVQSGAEVKKPGASVKVSCKASGYTFSNYGITWVRQAPGQGLEWM | 266 |
| | | GWISAYNGNTNYAQKFQGRVTMTRNTSISTAYMELSSLRSEDTAVYYC | |
| | | ARGPYYYYMDVWGKGTMVTVSSASGGGGSGGRASGGGGSEIVMTQSPL | |
| | | SLPVTPGEPASISCRSSQSLLYSNGYNYVDWYLQKPGQSPQLLIYLGS | |
| | | NRASGVPDRFSGSGSGTDFKLQISRVEAEDVGIYYCMQGRQFPYSFGQ | |
| | | GTKVEIK | |
| BCMA-8 | VH | EVQLLETGGGLVQPGGSLRLSCAVSGFALSNHGMSWVRRAPGKGLEWV | 267 |
| | | SGIVYSGSTYYAASVKGRFTISRDNSRNTLYLQMNSLRPEDTAIYYCS | |
| | | AHGGESDVWGQGTTVTVSS | |
| | VL | EIVLTQSPATLSVSPGESATLSCRASQSVSSNLAWYQQKPGQAPRLLI | 268 |
| | | YGASTRASGIPDRFSGSGSGTDFTLTISSLQAEDVAVYYCQQYGSSLT | |
| | | FGGGTKVEIK | |
| | scFv | EVQLLETGGGLVQPGGSLRLSCAVSGFALSNHGMSWVRRAPGKGLEWV | 269 |
| | | SGIVYSGSTYYAASVKGRFTISRDNSRNTLYLQMNSLRPEDTAIYYCS | |
| | | AHGGESDVWGQGTTVTVSSASGGGGSGGRASGGGGSEIVLTQSPATLS | |
| | | VSPGESATLSCRASQSVSSNLAWYQQKPGQAPRLLIYGASTRASGIPD | |
| | | RFSGSGSGTDFTLTISSLQAEDVAVYYCQQYGSSLTFGGGTKVEIK | |
| BCMA-9 | VH | EVQLVETGGGLVQPGGSLRLSCAVSGFALSNHGMSWVRRAPGKGLEWV | 270 |
| | | SGIVYSGSTYYAASVKGRFTISRDNSRNTLYLQMNSLRPEDTAIYYCS | |
| | | AHGGESDVWGQGTTVTVSS | |
| | VL | EIVMTQSPATLSVSPGERATLSCRASQSVSSKLAWYQQKPGQAPRLLM | 271 |
| | | YGASIRATGIPDRFSGSGSGTEFTLTISSLEPEDFAVYYCQQYGSSSW | |
| | | TFGQGTKVEIK | |
| | scFv | EVQLVETGGGLVQPGGSLRLSCAVSGFALSNHGMSWVRRAPGKGLEWV | 272 |
| | | SGIVYSGSTYYAASVKGRFTISRDNSRNTLYLQMNSLRPEDTAIYYCS | |
| | | AHGGESDVWGQGTTVTVSSASGGGGSGGRASGGGGSEIVMTQSPATLS | |
| | | VSPGERATLSCRASQSVSSKLAWYQQKPGQAPRLLMYGASIRATGIPD | |
| | | RFSGSGSGTEFTLTISSLEPEDFAVYYCQQYGSSSWTFGQGTKVEIK | |
| BCMA-10 | VH | EVQLVETGGGVVQPGGSLRLSCAVSGFALSNHGMSWVRRAPGKGLEWV | 273 |
| | | SGIVYSGSTYYAASVKGRFTISRDNSRNTLYLQMNSLRPEDTAIYYCS | |
| | | AHGGESDVWGQGTTVTVSS | |
| | VL | EIVLTQSPGTLSLSPGERATLSCRASQSVGSTNLAWYQQKPGQAPRLL | 274 |
| | | IYDASNRATGIPDRFSGGGSGTDFTLTISRLEPEDFAVYYCQQYGSSP | |
| | | PWTFGQGTKVEIK | |
| | scFv | EVQLVETGGGVVQPGGSLRLSCAVSGFALSNHGMSWVRRAPGKGLEWV | 275 |
| | | SGIVYSGSTYYAASVKGRFTISRDNSRNTLYLQMNSLRPEDTAIYYCS | |
| | | AHGGESDVWGQGTTVTVSSASGGGGSGGRASGGGGSEIVLTQSPGTLS | |
| | | LSPGERATLSCRASQSVGSTNLAWYQQKPGQAPRLLIYDASNRATGIP | |
| | | DRFSGGGSGTDFTLTISRLEPEDFAVYYCQQYGSSPPWTFGQGTKVEI | |
| | | K | |
| BCMA-11 | VH | QVQLVESGGGLVKPGGSLRLSCAASGFTFSDYYMSWIRQAPGKGLEWV | 276 |
| | | SYISSSGSTIYYADSVKGRFTISRDNAKNSLYLQMNSLRAEDTAVYYC | |
| | | ARESGDGMDVWGQGTTVTVSS | |
| | VL | DIQMTQSPSSLSASVGDRVTITCRASQSISSYLNWYQQKPGKAPKLLI | 277 |
| | | YAASSLQSGVPSRFSGSGSGTDFTLTISSLQPEDFATYYCQQSYTLAF | |
| | | GQGTKVDIK | |
| | scFv | QVQLVESGGGLVKPGGSLRLSCAASGFTFSDYYMSWIRQAPGKGLEWV | 278 |
| | | SYISSSGSTIYYADSVKGRFTISRDNAKNSLYLQMNSLRAEDTAVYYC | |
| | | ARESGDGMDVWGQGTTVTVSSASGGGGSGGRASGGGGSDIQMTQSPSS | |
| | | LSASVGDRVTITCRASQSISSYLNWYQQKPGKAPKLLIYAASSLQSGV | |
| | | PSRFSGSGSGTDFTLTISSLQPEDFATYYCQQSYTLAFGQGTKVDIK | |
| BCMA-12 | VH | QVQLVQSGGGLVKPGGSLRLSCAASGFTFSDYYMSWIRQAPGKGLEWV | 279 |
| | | SYISSSGNTIYYADSVKGRFTISRDNAKNSLYLQMNSLRAEDTAVYYC | |
| | | ARSTMVREDYWGQGTLVTVSS | |
| | VL | DIVLTQSPLSLPVTLGQPASISCKSSESLVHNSGKTYLNWFHQRPGQS | 280 |
| | | PRRLIYEVSNRDSGVPDRFTGSGSGTDFTLKISRVEAEDVGVYYCMQG | |
| | | THWPGTFGQGTKLEIK | |
| | scFv | QVQLVQSGGGLVKPGGSLRLSCAASGFTFSDYYMSWIRQAPGKGLEWV | 281 |
| | | SYISSSGNTIYYADSVKGRFTISRDNAKNSLYLQMNSLRAEDTAVYYC | |
| | | ARSTMVREDYWGQGTLVTVSSASGGGGSGGRASGGGGSDIVLTQSPLS | |
| | | LPVTLGQPASISCKSSESLVHNSGKTYLNWFHQRPGQSPRRLIYEVSN | |
| | | RDSGVPDRFTGSGSGTDFTLKISRVEAEDVGVYYCMQGTHWPGTFGQG | |
| | | TKLEIK | |
| BCMA-13 | VH | QVQLVESGGGLVQPGGSLRLSCAVSGFALSNHGMSWVRRAPGKGLEWV | 282 |
| | | SGIVYSGSTYYAASVKGRFTISRDNSRNTLYLQMNSLRPEDTAIYYCS | |
| | | AHGGESDVWGQGTTVTVSS | |
| | VL | DIRLTQSPSPLSASVGDRVTITCQASEDINKFLNWYHQTPGKAPKLLI | 283 |
| | | YDASTLQTGVPSRFSGSGSGTDFTLTINSLQPEDIGTYYCQQYESLPL | |
| | | TFGGGTKVEIK | |
| | scFv | QVQLVESGGGLVQPGGSLRLSCAVSGFALSNHGMSWVRRAPGKGLEWV | 284 |
| | | SGIVYSGSTYYAASVKGRFTISRDNSRNTLYLQMNSLRPEDTAIYYCS | |
| | | AHGGESDVWGQGTTVTVSSASGGGGSGGRASGGGGSDIRLTQSPSPLS | |
| | | ASVGDRVTITCQASEDINKFLNWYHQTPGKAPKLLIYDASTLQTGVPS | |
| | | RFSGSGSGTDFTLTINSLQPEDIGTYYCQQYESLPLTFGGGTKVEIK | |
| BCMA-14 | VH | EVQLVETGGGLVQPGGSLRLSCAVSGFALSNHGMSWVRRAPGKGLEWV | 270 |
| | | SGIVYSGSTYYAASVKGRFTISRDNSRNTLYLQMNSLRPEDTAIYYCS | |
| | | AHGGESDVWGQGTTVTVSS | |
| | VL | ETTLTQSPATLSVSPGERATLSCRASQSVGSNLAWYQQKPGQGPRLLI | 285 |
| | | YGASTRATGIPARFSGSGSGTEFTLTISSLQPEDFAVYYCQQYNDWLP | |
| | | VTFGQGTKVEIK | |
| | scFv | EVQLVETGGGLVQPGGSLRLSCAVSGFALSNHGMSWVRRAPGKGLEWV | 286 |
| | | SGIVYSGSTYYAASVKGRFTISRDNSRNTLYLQMNSLRPEDTAIYYCS | |
| | | AHGGESDVWGQGTTVTVSSASGGGGSGGRASGGGGSETTLTQSPATLS | |
| | | VSPGERATLSCRASQSVGSNLAWYQQKPGQGPRLLIYGASTRATGIPA | |
| | | RFSGSGSGTEFTLTISSLQPEDFAVYYCQQYNDWLPVTFGQGTKVEIK | |
| BCMA-15 | VH | EVQLVESGGGLVQPGGSLRLSCAVSGFALSNHGMSWVRRAPGKGLEWV | 246 |
| | | SGIVYSGSTYYAASVKGRFTISRDNSRNTLYLQMNSLRPEDTAIYYCS | |
| | | AHGGESDVWGQGTTVTVSS | |
| | VL | EIVLTQSPGTLSLSPGERATLSCRASQSIGSSSLAWYQQKPGQAPRLL | 287 |
| | | MYGASSRASGIPDRFSGSGSGTDFTLTISRLEPEDFAVYYCQQYAGSP | |
| | | PFTFGQGTKVEIK | |
| | scFv | EVQLVESGGGLVQPGGSLRLSCAVSGFALSNHGMSWVRRAPGKGLEWV | 288 |
| | | SGIVYSGSTYYAASVKGRFTISRDNSRNTLYLQMNSLRPEDTAIYYCS | |
| | | AHGGESDVWGQGTTVTVSSASGGGGSGGRASGGGGSEIVLTQSPGTLS | |
| | | LSPGERATLSCRASQSIGSSSLAWYQQKPGQAPRLLMYGASSRASGIP | |
| | | DRFSGSGSGTDFTLTISRLEPEDFAVYYCQQYAGSPPFTFGQGTKVEI | |
| | | K | |
| BCMA-16 | VH | QVQLQESGPGLVKPSETLSLTCTVSGGSISSSYYYWGWIRQPPGKGLE | 289 |
| | | WIGSIYYSGSAYYNPSLKSRVTISVDTSKNQFSLRLSSVTAADTAVYY | |
| | | CARHWQEWPDAFDIWGQGTMVTVSS | |
| | VL | ETTLTQSPAFMSATPGDKVIISCKASQDIDDAMNWYQQKPGEAPLFII | 290 |
| | | QSATSPVPGIPPRFSGSGFGTDFSLTINNIESEDAAYYFCLQHDNFPL | |
| | | TFGQGTKLEIK | |
| | scFv | QVQLQESGPGLVKPSETLSLTCTVSGGSISSSYYYWGWIRQPPGKGLE | 291 |
| | | WIGSIYYSGSAYYNPSLKSRVTISVDTSKNQFSLRLSSVTAADTAVYY | |
| | | CARHWQEWPDAFDIWGQGTMVTVSSGGGGSGGGGSGGGGSETTLTQSP | |
| | | AFMSATPGDKVIISCKASQDIDDAMNWYQQKPGEAPLFIIQSATSPVP | |
| | | GIPPRFSGSGFGTDFSLTINNIESEDAAYYFCLQHDNFPLTFGQGTKL | |
| | | EIK | |
| BCMA-17 | VH | QVNLRESGPALVKPTQTLTLTCTFSGFSLRTSGMCVSWIRQPPGKALE | 292 |
| | | WLARIDWDEDKFYSTSLKTRLTISKDTSDNQVVLRMTNMDPADTATYY | |
| | | CARSGAGGTSATAFDIWGPGTMVTVSS | |
| | VL | DIQMTQSPSSLSASVGDRVTITCRASQDIYNNLAWFQLKPGSAPRSLM | 293 |
| | | YAANKSQSGVPSRFSGSASGTDFTLTISSLQPEDFATYYCQHYYRFPY | |
| | | SFGQGTKLEIK | |
| | scFv | VNLRESGPALVKPTQTLTLTCTFSGFSLRTSGMCVSWIRQPPGKALEW | 294 |
| | | LARIDWDEDKFYSTSLKTRLTISKDTSDNQVVLRMTNMDPADTATYYC | |
| | | ARSGAGGTSATAFDIWGPGTMVTVSSGGGGSGGGGSGGGGSDIQMTQS | |
| | | PSSLSASVGDRVTITCRASQDIYNNLAWFQLKPGSAPRSLMYAANKSQ | |
| | | SGVPSRFSGSASGTDFTLTISSLQPEDFATYYCQHYYRFPYSFGQGTK | |
| | | LEIK | |
| BCMA-18 | VH | EVQLVESGGGLVKPGGSLRLSCAASGFTFSSYSMNWVRQAPGKGLEWV | 295 |
| | | SSISSSSSYIYYADSVKGRFTISRDNAKNSLYLQMNSLRAEDTAVYYC | |
| | | AKTIAAVYAFDIWGQGTTVTVSS | |
| | VL | EIVLTQSPLSLPVTPEEPASISCRSSQSLLHSNGYNYLDWYLQKPGQS | 296 |
| | | PQLLIYLGSNRASGVPDRFSGSGSGTDFTLKISRVEAEDVGVYYCMQA | |
| | | LQTPYTFGQGTKLEIK | |
| | scFv | EVQLVESGGGLVKPGGSLRLSCAASGFTFSSYSMNWVRQAPGKGLEWV | 297 |
| | | SSISSSSSYIYYADSVKGRFTISRDNAKNSLYLQMNSLRAEDTAVYYC | |
| | | AKTIAAVYAFDIWGQGTTVTVSSGGGGSGGGGSGGGGSEIVLTQSPLS | |
| | | LPVTPEEPASISCRSSQSLLHSNGYNYLDWYLQKPGQSPQLLIYLGSN | |
| | | RASGVPDRFSGSGSGTDFTLKISRVEAEDVGVYYCMQALQTPYTFGQG | |
| | | TKLEIK | |
| BCMA-19 | VH | EVQLVESGGGLVKPGGSLRLSCAASGFTFSDYYMSWIRQAPGKGLEWV | 298 |
| | | SYISSSGSTIYYADSVKGRFTISRDNAKNSLYLQMNSLRAEDTAVYYC | |
| | | ARDLRGAFDIWGQGTMVTVSS | |
| | VL | SYVLTQSPSVSAAPGYTATISCGGNNIGTKSVHWYQQKPGQAPLLVIR | 299 |
| | | DDSVRPSKIPGRFSGSNSGNMATLTISGVQAGDEADFYCQVWDSDSEH | |
| | | VVFGGGTKLTVL | |
| | scFv | EVQLVESGGGLVKPGGSLRLSCAASGFTFSDYYMSWIRQAPGKGLEWV | 300 |
| | | SYISSSGSTIYYADSVKGRFTISRDNAKNSLYLQMNSLRAEDTAVYYC | |
| | | ARDLRGAFDIWGQGTMVTVSSGGGGSGGGGSGGGGSSYVLTQSPSVSA | |
| | | APGYTATISCGGNNIGTKSVHWYQQKPGQAPLLVIRDDSVRPSKIPGR | |
| | | FSGSNSGNMATLTISGVQAGDEADFYCQVWDSDSEHVVFGGGTKLTVL | |
| BCMA-20 | VH | QVQLVQSGAEVKKPGASVKVSCKPSGYTVTSHYIHWVRRAPGQGLEWM | 301 |
| | | GMINPSGGVTAYSQTLQGRVTMTSDTSSSTVYMELSSLRSEDTAMYYC | |
| | | AREGSGSGWYFDFWGRGTLVTVSS | |
| | VL | SYVLTQPPSVSVSPGQTASITCSGDGLSKKYVSWYQQKAGQSPVVLIS | 302 |
| | | RDKERPSGIPDRFSGSNSADTATLTISGTQAMDEADYYCQAWDDTTVV | |
| | | FGGGTKLTVL | |
| | scFv | QVQLVQSGAEVKKPGASVKVSCKPSGYTVTSHYIHWVRRAPGQGLEWM | 303 |
| | | GMINPSGGVTAYSQTLQGRVTMTSDTSSSTVYMELSSLRSEDTAMYYC | |
| | | AREGSGSGWYFDFWGRGTLVTVSSGGGGSGGGGSGGGGSSYVLTQPPS | |
| | | VSVSPGQTASITCSGDGLSKKYVSWYQQKAGQSPVVLISRDKERPSYC | |
| | | GIPDRFSGSNSADTATLTISGTQAMDEADYQAWDDTTVVFGGGTKLTV | |
| | | L | |
| BCMA-21 | VH | QVQLQESGPGLVKPSQTLSLTCTVSGGSISSGGYYWSWIRQHPGKGLE | 304 |
| | | WIGYIYYSGSTYYNPSLKSRVTISVDTSKNQFSLKLSSVTAADTAVYY | |
| | | CARAGIAARLRGAFDIWGQGTMVTVSS | |
| | VL | DIVMTQSPSSVSASVGDRVIITCRASQGIRNWLAWYQQKPGKAPNLLI | 305 |
| | | YAASNLQSGVPSRFSGSGSGADFTLTISSLQPEDVATYYCQKYNSAPF | |
| | | TFGPGTKVDIK | |
| | scFv | QVQLQESGPGLVKPSQTLSLTCTVSGGSISSGGYYWSWIRQHPGKGLE | 306 |
| | | WIGYIYYSGSTYYNPSLKSRVTISVDTSKNQFSLKLSSVTAADTAVYY | |
| | | CARAGIAARLRGAFDIWGQGTMVTVSSGGGGSGGGGSGGGGSDIVMTQ | |
| | | SPSSVSASVGDRVIITCRASQGIRNWLAWYQQKPGKAPNLLIYAASNL | |
| | | QSGVPSRFSGSGSGADFTLTISSLQPEDVATYYCQKYNSAPFTFGPGT | |
| | | KVDIK | |
| BCMA-22 | VH | QVQLVQSGAEVKKPGSSVKVSCKASGGTFSSYAISWVRQAPGQGLEWM | 307 |
| | | GGIIPIFGTANYAQKFQGRVTITADESTSTAYMELSSLRSEDTAVYYC | |
| | | ARRGGYQLLRWDVGLLRSAFDIWGQGTMVTVSS | |
| | VL | SYVLTQPPSVSVAPGQTARITCGGNNIGSKSVHWYQQKPGQAPVLVLY | 308 |
| | | GKNNRPSGVPDRFSGSRSGTTASLTITGAQAEDEADYYCSSRDSSGDH | |
| | | LRVFGTGTKVTVL | |
| | scFv | QVQLVQSGAEVKKPGSSVKVSCKASGGTFSSYAISWVRQAPGQGLEWM | 309 |
| | | GGIIPIFGTANYAQKFQGRVTITADESTSTAYMELSSLRSEDTAVYYC | |
| | | ARRGGYQLLRWDVGLLRSAFDIWGQGTMVTVSSGGGGSGGGGSGGGGS | |
| | | SYVLTQPPSVSVAPGQTARITCGGNNIGSKSVHWYQQKPGQAPVLVLY | |
| | | GKNNRPSGVPDRFSGSRSGTTASLTITGAQAEDEADYYCSSRDSSGDH | |
| | | LRVFGTGTKVTVL | |
| BCMA-23 | VH | EVQLQQSGPGLVKPSQTLSLTCAISGDSVSSNSAAWNWIRQSPSRGLE | 310 |
| | | WLGRTYYRSKWYSFYAISLKSRIIINPDTSKNQFSLQLKSVTPEDTAV | |
| | | YYCARSSPEGLFLYWFDPWGQGTLVTVSS | |
| | VL | SSELTQDPAVSVALGQTIRITCQGDSLGNYYATWYQQKPGQAPVLVIY | 311 |
| | | GTNNRPSGIPDRFSASSSGNTASLTITGAQAEDEADYYCNSRDSSGHH | |
| | | LLFGTGTKVTVL | |
| | ScFv | EVQLQQSGPGLVKPSQTLSLTCAISGDSVSSNSAAWNWIRQSPSRGLE | 312 |
| | | WLGRTYYRSKWYSFYAISLKSRIIINPDTSKNQFSLQLKSVTPEDTAV | |
| | | YYCARSSPEGLFLYWFDPWGQGTLVTVSSGGDGSGGGGSGGGGSSSEL | |
| | | TQDPAVSVALGQTIRITCQGDSLGNYYATWYQQKPGQAPVLVIYGTNN | |
| | | RPSGIPDRFSASSSGNTASLTITGAQAEDEADYYCNSRDSSGHHLLFG | |
| | | TGTKVTVL | |
| BCMA-24 | VH | EVQLVESGGGLVQPGGSLRLSCAASGFTFSSYAMSWVRQAPGKGLEWV | 313 |
| | | SAISGSGGSTYYADSVKGRFTISRDNSKNTLYLQMNSLRAEDTAVYYC | |
| | | AKVEGSGSLDYWGQGTLVTVSS | |
| | VL | EIVMTQSPGTLSLSPGERATLSCRASQSVSSAYLAWYQQKPGQPPRLL | 314 |
| | | ISGASTRATGIPDRFGGSGSGTDFTLTISRLEPEDFAVYYCQHYGSSF | |
| | | NGSSLFTFGQGTRLEIK | |
| | scFv | EVQLVESGGGLVQPGGSLRLSCAASGFTFSSYAMSWVRQAPGKGLEWV | 315 |
| | | SAISGSGGSTYYADSVKGRFTISRDNSKNTLYLQMNSLRAEDTAVYYC | |
| | | AKVEGSGSLDYWGQGTLVTVSSGGGGSGGGGSGGGGSEIVMTQSPGTL | |
| | | SLSPGERATLSCRASQSVSSAYLAWYQQKPGQPPRLLISGASTRATGI | |
| | | PDRFGGSGSGTDFTLTISRLEPEDFAVYYCQHYGSSFNGSSLFTFGQG | |
| | | TRLEIK | |
| | VH | EVQLVETGGGLVQPGGSLRLSCAASGITFSRYPMSWVRQAPGKGLEWV | 316 |
| | | SGISDSGVSTYYADSAKGRFTISRDNSKNTLFLQMSSLRDEDTAVYYC | |
| | | VTRAGSEASDIWGQGTMVTVSS | |
| BCMA-25 | VL | EIVLTQSPATLSLSPGERATLSCRASQSVSNSLAWYQQKPGQAPRLLI | 317 |
| | | YDASSRATGIPDRFSGSGSGTDFTLTISRLEPEDFAIYYCQQFGTSSG | |
| | | LTFGGGTKLEIK | |
| | scFv | EVQLVETGGGLVQPGGSLRLSCAASGITFSRYPMSWVRQAPGKGLEWV | 318 |
| | | SGISDSGVSTYYADSAKGRFTISRDNSKNTLFLQMSSLRDEDTAVYYC | |
| | | VTRAGSEASDIWGQGTMVTVSSGGGGSGGGGSGGGGSEIVLTQSPATL | |
| | | SLSPGERATLSCRASQSVSNSLAWYQQKPGQAPRLLIYDASSRATGIP | |
| | | DRFSGSGSGTDFTLTISRLEPEDFAIYYCQQFGTSSGLTFGGGTKLEI | |
| | | K | |
| BCMA-26 | VH | QVQLVESGGGLVQPGGSLRLSCAASGFTFSSYAMSWVRQAPGKGLEWV | 319 |
| | | SAISGSGGSTYYADSVKGRFTISRDNAKNSLYLQMNSLRAEDTAIYYC | |
| | | ARATYKRELRYYYGMDVWGQGTMVTVSS | |
| | VL | EIVMTQSPGTVSLSPGERATLSCRASQSVSSSFLAWYQQKPGQAPRLL | 320 |
| | | IYGASSRATGIPDRFSGSGSGTDFTLTISRLEPEDSAVYYCQQYHSSP | |
| | | SWTFGQGTRLEIK | |
| | scFv | QVQLVESGGGLVQPGGSLRLSCAASGFTFSSYAMSWVRQAPGKGLEWV | 321 |
| | | SAISGSGGSTYYADSVKGRFTISRDNAKNSLYLQMNSLRAEDTAIYYC | |
| | | ARATYKRELRYYYGMDVWGQGTMVTVSSGGGGSGGGGSGGGGSEIVMT | |
| | | QSPGTVSLSPGERATLSCRASQSVSSSFLAWYQQKPGQAPRLLIYGAS | |
| | | SRATGIPDRFSGSGSGTDFTLTISRLEPEDSAVYYCQQYHSSPSWTFG | |
| | | QGTRLEIK | |
| BCMA-27 | VH | EVQLVETGGGLVQPGGSLRLSCAASGFTFSSYAMSWVRQAPGKGLEWV | 322 |
| | | SAISGSGGSTYYADSVKGRFTISRDNSKNTLYLQMNTLKAEDTAVYYC | |
| | | ARATYKRELRYYYGMDVWGQGTTVTVSS | |
| | VL | EIVLTQSPSTLSLSPGESATLSCRASQSVSTTFLAWYQQKPGQAPRLL | 323 |
| | | IYGSSNRATGIPDRFSGSGSGTDFTLTIRRLEPEDFAVYYCQQYHSSP | |
| | | SWTFGQGTKVEIK | |
| | scFv | EVQLVETGGGLVQPGGSLRLSCAASGFTFSSYAMSWVRQAPGKGLEWV | 324 |
| | | SAISGSGGSTYYADSVKGRFTISRDNSKNTLYLQMNTLKAEDTAVYYC | |
| | | ARATYKRELRYYYGMDVWGQGTTVTVSSGGGGSGGGGSGGGGSEIVLT | |
| | | QSPSTLSLSPGESATLSCRASQSVSTTFLAWYQQKPGQAPRLLIYGSS | |
| | | NRATGIPDRFSGSGSGTDFTLTIRRLEPEDFAVYYCQQYHSSPSWTFG | |
| | | QGTKVEIK | |
| BCMA-28 | VH | EVQLVETGGGLVQPGRSLRLSCAASGFTFDDYAMHWVRQAPGKGLEWV | 325 |
| | | SGISWNSGSIGYADSVKGRFTISRDNAKNSLYLQMNSLRDEDTAVYYC | |
| | | ARVGKAVPDVWGQGTTVTVSS | |
| | VL | DIVMTQTPSSLSASVGDRVTITCRASQSISSYLNWYQQKPGKAPKLLI | 326 |
| | | YAASSLQSGVPSRFSGSGSGTDFTLTISSLQPEDFATYYCQQSYSTPY | |
| | | SFGQGTRLEIK | |
| | scFv | EVQLVETGGGLVQPGRSLRLSCAASGFTFDDYAMHWVRQAPGKGLEWV | 327 |
| | | SGISWNSGSIGYADSVKGRFTISRDNAKNSLYLQMNSLRDEDTAVYYC | |
| | | ARVGKAVPDVWGQGTTVTVSSGGGGSGGGGSGGGGSDIVMTQTPSSLS | |
| | | ASVGDRVTITCRASQSISSYLNWYQQKPGKAPKLLIYAASSLQSGVPS | |
| | | RFSGSGSGTDFTLTISSLQPEDFATYYCQQSYSTPYSFGQGTRLEIK | |
| BCMA-29 | VH | EVQLVESGGGLVQPGRSLRLSCTASGFTFDDYAMHWVRQRPGKGLEWV | 328 |
| | | ASINWKGNSLAYGDSVKGRFAISRDNAKNTVFLQMNSLRTEDTAVYYC | |
| | | ASHQGVAYYNYAMDVWGRGTLVTVSS | |
| | VL | EIVLTQSPGTLSLSPGERATLSCRATQSIGSSFLAWYQQRPGQAPRLL | 329 |
| | | IYGASQRATGIPDRFSGRGSGTDFTLTISRVEPEDSAVYYCQHYESSP | |
| | | SWTFGQGTKVEIK | |
| | scFv | EVQLVESGGGLVQPGRSLRLSCTASGFTFDDYAMHWVRQRPGKGLEWV | 330 |
| | | ASINWKGNSLAYGDSVKGRFAISRDNAKNTVFLQMNSLRTEDTAVYYC | |
| | | ASHQGVAYYNYAMDVWGRGTLVTVSSGGGGSGGGGSGGGGSEIVLTQS | |
| | | PGTLSLSPGERATLSCRATQSIGSSFLAWYQQRPGQAPRLLIYGASQR | |
| | | ATGIPDRFSGRGSGTDFTLTISRVEPEDSAVYYCQHYESSPSWTFGQG | |
| | | TKVEIK | |
| BCMA-30 | VH | EVQLVESGGGLVQPGGSLRLSCAASGFTFSSYAMSWVRQAPGKGLEWV | 331 |
| | | SAISGSGGSTYYADSVKGRFTISRDNSKNTLYLQMNSLRAEDTAVYYC | |
| | | AKVVRDGMDVWGQGTTVTVSS | |
| | VL | EIVLTQSPATLSLSPGERATLSCRASQSVSSSYLAWYQQKPGQAPRLL | 332 |
| | | IYGASSRATGIPDRFSGNGSGTDFTLTISRLEPEDFAVYYCQQYGSPP | |
| | | RFTFGPGTKVDIK | |
| | scFv | EVQLVESGGGLVQPGGSLRLSCAASGFTFSSYAMSWVRQAPGKGLEWV | 333 |
| | | SAISGSGGSTYYADSVKGRFTISRDNSKNTLYLQMNSLRAEDTAVYYC | |
| | | AKVVRDGMDVWGQGTTVTVSSGGGGSGGGGSGGGGSEIVLTQSPATLS | |
| | | LSPGERATLSCRASQSVSSSYLAWYQQKPGQAPRLLIYGASSRATGIP | |
| | | DRFSGNGSGTDFTLTISRLEPEDFAVYYCQQYGSPPRFTFGPGTKVDI | |
| | | K | |
| BCMA-31 | VH | EVQLLESGGGLVQPGGSLRLSCAASGFTFSSYAMSWVRQAPGKGLEWV | 334 |
| | | SAISGSGGSTYYADSVKGRFTISRDNSKNTLYLQMNSLRAEDTAVYYC | |
| | | AKIPQTGTFDYWGQGTLVTVSS | |
| | VL | EIVLTQSPGTLSLSPGERATLSCRASQSVSSSYLAWYQQRPGQAPRLL | 335 |
| | | IYGASSRATGIPDRFSGSGSGTDFTLTISRLEPEDFAVYYCQHYGSSP | |
| | | SWTFGQGTRLEIK | |
| | scFv | EVQLLESGGGLVQPGGSLRLSCAASGFTFSSYAMSWVRQAPGKGLEWV | 336 |
| | | SAISGSGGSTYYADSVKGRFTISRDNSKNTLYLQMNSLRAEDTAVYYC | |
| | | AKIPQTGTFDYWGQGTLVTVSSGGGGSGGGGSGGGGSEIVLTQSPGTL | |
| | | SLSPGERATLSCRASQSVSSSYLAWYQQRPGQAPRLLIYGASSRATGI | |
| | | PDRFSGSGSGTDFTLTISRLEPEDFAVYYCQHYGSSPSWTFGQGTRLE | |
| | | IK | |
| BCMA-32 | VH | EVQLVETGGGLVQPGGSLRLSCAASGFTFSSYAMSWVRQAPGKGLEWV | 337 |
| | | SAISGSGGSTYYADSVKGRFTMSRENDKNSVFLQMNSLRVEDTGVYYC | |
| | | ARANYKRELRYYYGMDVWGQGTMVTVSS | |
| | VL | EIVMTQSPGTLSLSPGESATLSCRASQRVASNYLAWYQHKPGQAPSLL | 338 |
| | | ISGASSRATGVPDRFSGSGSGTDFTLAISRLEPEDSAVYYCQHYDSSP | |
| | | SWTFGQGTKVEIK | |
| | scFv | EVQLVETGGGLVQPGGSLRLSCAASGFTFSSYAMSWVRQAPGKGLEWV | 339 |
| | | SAISGSGGSTYYADSVKGRFTMSRENDKNSVFLQMNSLRVEDTGVYYC | |
| | | ARANYKRELRYYYGMDVWGQGTMVTVSSGGGGSGGGGSGGGGSEIVMT | |
| | | QSPGTLSLSPGESATLSCRASQRVASNYLAWYQHKPGQAPSLLISGAS | |
| | | SRATGVPDRFSGSGSGTDFTLAISRLEPEDSAVYYCQHYDSSPSWTFG | |
| | | QGTKVEIK | |
| BCMA-33 | VH | EVQLLETGGGLVQPGGSLRLSCAASGFSFSSYAMSWVRQAPGKGLEWV | 340 |
| | | SAISGSGGSTYYADSVKGRFTISRDNSKNTLYLQMNSLRAEDTAVYYC | |
| | | AKALVGATGAFDIWGQGTLVTVSS | |
| | VL | EIVLTQSPGTLSLSPGERATLSCRASQSLSSNFLAWYQQKPGQAPGLL | 341 |
| | | IYGASNWATGTPDRFSGSGSGTDFTLTITRLEPEDFAVYYCQYYGTSP | |
| | | MYTFGQGTKVEIK | |
| | scFv | EVQLLETGGGLVQPGGSLRLSCAASGFSFSSYAMSWVRQAPGKGLEWV | 342 |
| | | SAISGSGGSTYYADSVKGRFTISRDNSKNTLYLQMNSLRAEDTAVYYC | |
| | | AKALVGATGAFDIWGQGTLVTVSSGGGGSGGGGSGGGGSEIVLTQSPG | |
| | | TLSLSPGERATLSCRASQSLSSNFLAWYQQKPGQAPGLLIYGASNWAT | |
| | | GTPDRFSGSGSGTDFTLTITRLEPEDFAVYYCQYYGTSPMYTFGQGTK | |
| | | VEIK | |
| BCMA-34 | VH | EVQLLESGGGLVQPGGSLRLSCAASGFTFSSYAMSWVRQAPGKGLEWV | 343 |
| | | SAISGSGGSTYYADSVKGRFTISRDNSKNTLYLQMNSLRAEDTAVYYC | |
| | | VLWFGEGFDPWGQGTLVTVSS | |
| | VL | DIVLTQSPLSLPVTPGEPASISCRSSQSLLHSNGYNYLDWYLQKPGQS | 344 |
| | | PQLLIYLGSNRASGVPDRFSGSGSGTDFTLKISRVEAEDVGVYYCMQA | |
| | | LQTPLTFGGGTKVDIK | |
| | scFv | EVQLLESGGGLVQPGGSLRLSCAASGFTFSSYAMSWVRQAPGKGLEWV | 345 |
| | | SAISGSGGSTYYADSVKGRFTISRDNSKNTLYLQMNSLRAEDTAVYYC | |
| | | VLWFGEGFDPWGQGTLVTVSSGGGGSGGGGSGGGGSDIVLTQSPLSLP | |
| | | VTPGEPASISCRSSQSLLHSNGYNYLDWYLQKPGQSPQLLIYLGSNRA | |
| | | SGVPDRFSGSGSGTDFTLKISRVEAEDVGVYYCMQALQTPLTFGGGTK | |
| | | VDIK | |
| BCMA-35 | VH | QVQLVESGGGLVQPGGSLRLSCAASGFTFSSYAMSWVRQAPGKGLEWV | 346 |
| | | SAISGSGGSTYYADSVKGRFTISRDNSKNTLYLQMNSLRAEDTAVYYC | |
| | | AKVGYDSSGYYRDYYGMDVWGQGTTVTVSS | |
| | VL | EIVLTQSPGTLSLSPGERATLSCRASQSVSSSYLAWYQQKPGQAPRLL | 347 |
| | | IYGTSSRATGISDRFSGSGSGTDFTLTISRLEPEDFAVYYCQHYGNSP | |
| | | PKFTFGPGTKLEIK | |
| | scFv | QVQLVESGGGLVQPGGSLRLSCAASGFTFSSYAMSWVRQAPGKGLEWV | 348 |
| | | SAISGSGGSTYYADSVKGRFTISRDNSKNTLYLQMNSLRAEDTAVYYC | |
| | | AKVGYDSSGYYRDYYGMDVWGQGTTVTVSSGGGGSGGGGSGGGGSEIV | |
| | | LTQSPGTLSLSPGERATLSCRASQSVSSSYLAWYQQKPGQAPRLLIYG | |
| | | TSSRATGISDRFSGSGSGTDFTLTISRLEPEDFAVYYCQHYGNSPPKF | |
| | | TFGPGTKLEIK | |
| BCMA-36 | VH | EVQLVESGGGLVQPGGSLRLSCAASGFTFSSYAMSWVRQAPGKGLEWV | 349 |
| | | SAISGSGGSTYYADSVKGRFTISRDNSKNTLYLQMNSLRAEDTAVYYC | |
| | | AKMGWSSGYLGAFDIWGQGTTVTVSS | |
| | VL | EIVLTQSPGTLSLSPGERATLSCRASQSVASSFLAWYQQKPGQAPRLL | 350 |
| | | IYGASGRATGIPDRFSGSGSGTDFTLTISRLEPEDFAVYYCQHYGGSP | |
| | | RLTFGGGTKVDIK | |
| | scFv | EVQLVESGGGLVQPGGSLRLSCAASGFTFSSYAMSWVRQAPGKGLEWV | 351 |
| | | SAISGSGGSTYYADSVKGRFTISRDNSKNTLYLQMNSLRAEDTAVYYC | |
| | | AKMGWSSGYLGAFDIWGQGTTVTVSSGGGGSGGGGSGGGGSEIVLTQS | |
| | | PGTLSLSPGERATLSCRASQSVASSFLAWYQQKPGQAPRLLIYGASGR | |
| | | ATGIPDRFSGSGSGTDFTLTISRLEPEDFAVYYCQHYGGSPRLTFGGG | |
| | | TKVDIK | |
| BCMA-37 | VH | QIQLVQSGPDLKKPGETVKLSCKASGYTFTNFGMNWVKQAPGKGFKWM | 352 |
| | | AWINTYTGESYFADDFKGRFAFSVETSATTAYLQINNLKTEDTATYFC | |
| | | ARGEIYYGYDGGFAYWGQGTLVTVSA | |
| | VL | DVVMTQSHRFMSTSVGDRVSITCRASQDVNTAVSWYQQKPGQSPKLLI | 353 |
| | | FSASYRYTGVPDRFTGSGSGADFTLTISSVQAEDLAVYYCQQHYSTPW | |
| | | TFGGGTKLDIK | |
| | scFv | QIQLVQSGPDLKKPGETVKLSCKASGYTFTNFGMNWVKQAPGKGFKWM | 354 |
| | | AWINTYTGESYFADDFKGRFAFSVETSATTAYLQINNLKTEDTATYFC | |
| | | ARGEIYYGYDGGFAYWGQGTLVTVSAGGGGSGGGGSGGGGSDVVMTQS | |
| | | HRFMSTSVGDRVSITCRASQDVNTAVSWYQQKPGQSPKLLIFSASYRY | |
| | | TGVPDRFTGSGSGADFTLTISSVQAEDLAVYYCQQHYSTPWTFGGGTK | |
| | | LDIK | |
| BCMA-38 | VH | QIQLVQSGPELKKPGETVKISCKASGYTFTDYSINWVKRAPGKGLKWM | 355 |
| | | GWINTETREPAYAYDFRGRFAFSLETSASTAYLQINNLKYEDTATYFC | |
| | | ALDYSYAMDYWGQGTSVTVSS | |
| | VL | DIVLTQSPASLAMSLGKRATISCRASESVSVIGAHLIHWYQQKPGQPP | 356 |
| | | KLLIYLASNLETGVPARFSGSGSGTDFTLTIDPVEEDDVAIYSCLQSR | |
| | | IFPRTFGGGTKLEIK | |
| | scFv | QIQLVQSGPELKKPGETVKISCKASGYTFTDYSINWVKRAPGKGLKWM | 357 |
| | | GWINTETREPAYAYDFRGRFAFSLETSASTAYLQINNLKYEDTATYFC | |
| | | ALDYSYAMDYWGQGTSVTVSSGGGGSGGGGSGGGGSQIQLVQSGPELK | |
| | | KPGETVKISCKASGYTFTDYSINWVKRAPGKGLKWMGWINTETREPAY | |
| | | AYDFRGRFAFSLETSASTAYLQINNLKYEDTATYFCALDYSYAMDYWG | |
| | | QGTSVTVSS | |
| BCMA-39 | VH | QIQLVQSGPELKKPGETVKISCKASGYTFRHYSMNWVKQAPGKGLKWM | 358 |
| | | GRINTESGVPIYADDFKGRFAFSVETSASTAYLVINNLKDEDTASYFC | |
| | | SNDYLYSLDFWGQGTALTVSS | |
| | VL | DIVLTQSPPSLAMSLGKRATISCRASESVTILGSHLIYWYQQKPGQPP | 359 |
| | | TLLIQLASNVQTGVPARFSGSGSRTDFTLTIDPVEEDDVAVYYCLQSR | |
| | | TIPRTFGGGTKLEIK | |
| | scFv | QIQLVQSGPELKKPGETVKISCKASGYTFRHYSMNWVKQAPGKGLKWM | 360 |
| | | GRINTESGVPIYADDFKGRFAFSVETSASTAYLVINNLKDEDTASYFC | |
| | | SNDYLYSLDFWGQGTALTVSSGGGGSGGGGSGGGGSDIVLTQSPPSLA | |
| | | MSLGKRATISCRASESVTILGSHLIYWYQQKPGQPPTLLIQLASNVQT | |
| | | GVPARFSGSGSRTDFTLTIDPVEEDDVAVYYCLQSRTIPRTFGGGTKL | |
| | | EIK | |
| BCMA-40 | VH | QIQLVQSGPELKKPGETVKISCKASGYTFTHYSMNWVKQAPGKGLKWM | 361 |
| | | GRINTETGEPLYADDFKGRFAFSLETSASTAYLVINNLKNEDTATFFC | |
| | | SNDYLYSCDYWGQGTTLTVSS | |
| | VL | DIVLTQSPPSLAMSLGKRATISCRASESVTILGSHLIYWYQQKPGQPP | 359 |
| | | TLLIQLASNVQTGVPARFSGSGSRTDFTLTIDPVEEDDVAVYYCLQSR | |
| | | TIPRTFGGGTKLEIK | |
| | scFv | QIQLVQSGPELKKPGETVKISCKASGYTFTHYSMNWVKQAPGKGLKWM | 362 |
| | | GRINTETGEPLYADDFKGRFAFSLETSASTAYLVINNLKNEDTATFFC | |
| | | SNDYLYSCDYWGQGTTLTVSSGGGGSGGGGSGGGGSDIVLTQSPPSLA | |
| | | MSLGKRATISCRASESVTILGSHLIYWYQQKPGQPPTLLIQLASNVQT | |
| | | GVPARFSGSGSRTDFTLTIDPVEEDDVAVYYCLQSRTIPRTFGGGTKL | |
| | | EIK | |

TABLE 11B
BCMA Binders-Light chain CDR sequences according to Kabat numbering scheme

| | | SEQ | | SEQ | | SEQ |
|---|---|---|---|---|---|---|
| Antibody | CDR-L1 | ID NO: | CDR-L2 | ID NO: | CDR-L3 | ID NO: |
| BCMA-1 | RASQSISSYLN | 363 | AASSLQS | 395 | QQSYSTPYT | 424 |
| BCMA-2 | RASQSISSSFLA | 364 | GASRRAT | 396 | QQYHSSPSWT | 425 |
| BCMA-3 | RSSQSLLHSNGYNYLD | 365 | LGSNRAS | 397 | MQALQTPYT | 426 |
| BCMA-4 | KSSQSLLRNDGKTPLY | 366 | EVSNRFS | 398 | MQNIQFPS | 427 |
| BCMA-5 | RSSQSLLHSNGYNYLN | 367 | LGSKRAS | 399 | MQALQTPYT | 426 |
| BCMA-6 | RASQSISSYLN | 363 | GASTLAS | 400 | QQSYKRAS | 428 |
| BCMA-7 | RSSQSLLYSNGYNYVD | 368 | LGSNRAS | 397 | MQGRQFPYS | 429 |
| BCMA-8 | RASQSVSSNLA | 369 | GASTRAS | 401 | QQYGSSLT | 430 |
| BCMA-9 | RASQSVSSKLA | 370 | GASIRAT | 402 | QQYGSSSWT | 431 |
| BCMA-10 | RASQSVGSTNLA | 371 | DASNRAT | 157 | QQYGSSPPWT | 432 |
| BCMA-11 | RASQSISSYLN | 363 | AASSLQS | 395 | QQSYTLA | 433 |
| BCMA-12 | KSSESLVHNSGKTYLN | 372 | EVSNRDS | 403 | MQGTHWPGT | 434 |
| BCMA-13 | QASEDINKFLN | 373 | DASTLQT | 404 | QQYESLPLT | 435 |
| BCMA-14 | RASQSVGSNLA | 374 | GASTRAT | 405 | QQYNDWLPVT | 436 |
| BCMA-15 | RASQSIGSSSLA | 375 | GASSRAS | 406 | QQYAGSPPFT | 437 |
| BCMA-16 | KASQDIDDAMN | 376 | SATSPVP | 407 | LQHDNFPLT | 438 |
| BCMA-17 | RASQDIYNNLA | 377 | AANKSQS | 408 | QHYYRFPYS | 439 |
| BCMA-18 | RSSQSLLHSNGYNYLD | 365 | LGSNRAS | 397 | MQALQTPYT | 426 |
| BCMA-19 | GGNNIGTKSVH | 378 | DDSVRPS | 409 | QVWDSDSEHVV | 440 |
| BCMA-20 | SGDGLSKKYVS | 379 | RDKERPS | 410 | QAWDDTTVV | 441 |
| BCMA-21 | RASQGIRNWLA | 380 | AASNLQS | 411 | QKYNSAPFT | 442 |
| BCMA-22 | GGNNIGSKSVH | 381 | GKNNRPS | 412 | SSRDSSGDHLRV | 443 |
| BCMA-23 | QGDSLGNYYAT | 382 | GTNNRPS | 413 | NSRDSSGHHLL | 444 |
| BCMA-24 | RASQSVSSAYLA | 383 | GASTRAT | 405 | QHYGSSFNGSSLFT | 445 |
| BCMA-25 | RASQSVSNSLA | 384 | DASSRAT | 414 | QQFGTSSGLT | 446 |
| BCMA-26 | RASQSVSSSFLA | 385 | GASSRAT | 415 | QQYHSSPSWT | 425 |
| BCMA-27 | RASQSVSTTFLA | 386 | GSSNRAT | 416 | QQYHSSPSWT | 425 |
| BCMA-28 | RASQSISSYLN | 363 | AASSLQS | 395 | QQSYSTPYS | 447 |
| BCMA-29 | RATQSIGSSFLA | 387 | GASQRAT | 417 | QHYESSPSWT | 448 |
| BCMA-30 | RASQSVSSSYLA | 388 | GASSRAT | 415 | QQYGSPPRFT | 449 |
| BCMA-31 | RASQSVSSSYLA | 388 | GASSRAT | 415 | QHYGSSPSWT | 450 |
| BCMA-32 | RASQRVASNYLA | 389 | GASSRAT | 415 | QHYDSSPSWT | 451 |
| BCMA-33 | RASQSLSSNFLA | 390 | GASNWAT | 418 | QYYGTSPMYT | 452 |
| BCMA-34 | RSSQSLLHSNGYNYLD | 365 | LGSNRAS | 397 | MQALQTPLT | 453 |
| BCMA-35 | RASQSVSSSYLA | 388 | GTSSRAT | 419 | QHYGNSPPKFT | 454 |
| BCMA-36 | RASQSVASSFLA | 391 | GASGRAT | 420 | QHYGGSPRLT | 455 |
| BCMA-37 | RASQDVNTAVS | 392 | SASYRYT | 421 | QQHYSTPVVT | 456 |
| BCMA-38 | RASESVSVIGAHLIH | 393 | LASNLET | 422 | LQSRIFPRT | 457 |
| BCMA-39 | RASESVTILGSHLIY | 394 | LASNVQT | 423 | LQSRTIPRT | 458 |
| BCMA-40 | RASESVTILGSHLIY | 394 | LASNVQT | 423 | LQSRTIPRT | 458 |

TABLE 11C
BCMA Binders-Light chain CDR sequences according to Chothia numbering scheme

| Antibody | CDR-L1 | SEQ ID NO: | CDR-L2 | SEQ ID NO: | CDR-L3 | SEQ ID NO: |
|---|---|---|---|---|---|---|
| BCMA-1 | SQSISSY | 459 | AAS | 490 | SYSTPY | 503 |
| BCMA-2 | SQSISSSF | 460 | GAS | 491 | YHSSPSW | 504 |
| BCMA-3 | SQSLLHSNGYNY | 461 | LGS | 492 | ALQTPY | 505 |
| BCMA-4 | SQSLLRNDGKTP | 462 | EVS | 493 | NIQFP | 506 |
| BCMA-5 | SQSLLHSNGYNY | 461 | LGS | 492 | ALQTPY | 505 |
| BCMA-6 | SQSISSY | 459 | GAS | 491 | SYKRA | 507 |
| BCMA-7 | SQSLLYSNGYNY | 463 | LGS | 492 | GRQFPY | 508 |
| BCMA-8 | SQSVSSN | 464 | GAS | 491 | YGSSL | 509 |
| BCMA-9 | SQSVSSK | 465 | GAS | 491 | YGSSSW | 510 |
| BCMA-10 | SQSVGSTN | 466 | DAS | 216 | YGSSPPW | 511 |
| BCMA-11 | SQSISSY | 459 | AAS | 490 | SYTL | 512 |
| BCMA-12 | SESLVHNSGKTY | 467 | EVS | 493 | GTHWPG | 513 |
| BCMA-13 | SEDINKF | 468 | DAS | 216 | YESLPL | 514 |
| BCMA-14 | SQSVGSN | 469 | GAS | 491 | YNDWLPV | 515 |
| BCMA-15 | SQSIGSSS | 470 | GAS | 491 | YAGSPPF | 516 |
| BCMA-16 | SQDIDDA | 471 | SAT | 494 | HDNFPL | 517 |
| BCMA-17 | SQDIYNN | 472 | AAN | 495 | YYRFPY | 518 |
| BCMA-18 | SQSLLHSNGYNY | 461 | LGS | 492 | ALQTPY | 505 |
| BCMA-19 | NNIGTKS | 473 | DDS | 496 | WDSDSEHV | 519 |
| BCMA-20 | DGLSKKY | 474 | RDK | 497 | WDDTTV | 520 |
| BCMA-21 | SQGIRNW | 475 | AAS | 490 | YNSAPF | 521 |
| BCMA-22 | NNIGSKS | 476 | GKN | 498 | RDSSGDHLR | 522 |
| BCMA-23 | DSLGNYY | 477 | GTN | 212 | RDSSGHHL | 523 |
| BCMA-24 | SQSVSSAY | 478 | GAS | 491 | YGSSFNGSSLF | 524 |
| BCMA-25 | SQSVSNS | 479 | DAS | 216 | FGTSSGL | 525 |
| BCMA-26 | SQSVSSSF | 480 | GAS | 491 | YHSSPSW | 504 |
| BCMA-27 | SQSVSTTF | 481 | GSS | 499 | YHSSPSW | 504 |
| BCMA-28 | SQSISSY | 459 | AAS | 490 | SYSTPY | 503 |
| BCMA-29 | TQSIGSSF | 482 | GAS | 491 | YESSPSW | 526 |
| BCMA-30 | SQSVSSSY | 483 | GAS | 491 | YGSPPRF | 527 |
| BCMA-31 | SQSVSSSY | 483 | GAS | 491 | YGSSPSW | 528 |
| BCMA-32 | SQRVASNY | 484 | GAS | 491 | YDSSPSW | 529 |
| BCMA-33 | SQSLSSNF | 485 | GAS | 491 | YGTSPMY | 530 |
| BCMA-34 | SQSLLHSNGYNY | 461 | LGS | 492 | ALQTPL | 531 |
| BCMA-35 | SQSVSSSY | 483 | GTS | 500 | YGNSPPKF | 532 |
| BCMA-36 | SQSVASSF | 486 | GAS | 491 | YGGSPRL | 533 |
| BCMA-37 | SQDVNTA | 487 | SAS | 501 | HYSTPW | 534 |
| BCMA-38 | SESVSVIGAHL | 488 | LAS | 502 | SRIFPR | 535 |
| BCMA-39 | SESVTILGSHL | 489 | LAS | 502 | SRTIPR | 536 |
| BCMA-40 | SESVTILGSHL | 489 | LAS | 502 | SRTIPR | 536 |

TABLE 11D
BCMA Binders - Light chain CDR sequences according to combination
of Kabat and Chothia numbering schemes

| | | SEQ | | SEQ | | SEQ |
|---|---|---|---|---|---|---|
| | | ID | | ID | | ID |
| Antibody | CDR-L1 | NO: | CDR-L2 | NO: | CDR-L3 | NO: |
| BCMA-1 | RASQSISSYLN | 363 | AASSLQS | 395 | QQSYSTPYT | 424 |
| BCMA-2 | RASQSISSSFLA | 364 | GASRRAT | 396 | QQYHSSPSWT | 425 |
| BCMA-3 | RSSQSLLHSNGYNYLD | 365 | LGSNRAS | 397 | MQALQTPYT | 426 |
| BCMA-4 | KSSQSLLRNDGKTPLY | 366 | EVSNRFS | 398 | MQNIQFPS | 427 |
| BCMA-5 | RSSQSLLHSNGYNYLN | 367 | LGSKRAS | 399 | MQALQTPYT | 426 |
| BCMA-6 | RASQSISSYLN | 363 | GASTLAS | 400 | QQSYKRAS | 428 |
| BCMA-7 | RSSQSLLYSNGYNYVD | 368 | LGSNRAS | 397 | MQGRQFPYS | 429 |
| BCMA-8 | RASQSVSSNLA | 369 | GASTRAS | 401 | QQYGSSLT | 430 |
| BCMA-9 | RASQSVSSKLA | 370 | GASIRAT | 402 | QQYGSSSWT | 431 |
| BCMA-10 | RASQSVGSTNLA | 371 | DASNRAT | 157 | QQYGSSPPWT | 432 |
| BCMA-11 | RASQSISSYLN | 363 | AASSLQS | 395 | QQSYTLA | 433 |
| BCMA-12 | KSSESLVHNSGKTYLN | 372 | EVSNRDS | 403 | MQGTHWPGT | 434 |
| BCMA-13 | QASEDINKFLN | 373 | DASTLQT | 404 | QQYESLPLT | 435 |
| BCMA-14 | RASQSVGSNLA | 374 | GASTRAT | 405 | QQYNDWLPVT | 436 |
| BCMA-15 | RASQSIGSSSLA | 375 | GASSRAS | 406 | QQYAGSPPFT | 437 |
| BCMA-16 | KASQDIDDAMN | 376 | SATSPVP | 407 | LQHDNFPLT | 438 |
| BCMA-17 | RASQDIYNNLA | 377 | AANKSQS | 408 | QHYYRFPYS | 439 |
| BCMA-18 | RSSQSLLHSNGYNYLD | 365 | LGSNRAS | 397 | MQALQTPYT | 426 |
| BCMA-19 | GGNNIGTKSVH | 378 | DDSVRPS | 409 | QVWDSDSEHVV | 440 |
| BCMA-20 | SGDGLSKKYVS | 379 | RDKERPS | 410 | QAWDDTTVV | 441 |
| BCMA-21 | RASQGIRNWLA | 380 | AASNLQS | 411 | QKYNSAPFT | 442 |
| BCMA-22 | GGNNIGSKSVH | 381 | GKNNRPS | 412 | SSRDSSGDHLRV | 443 |
| BCMA-23 | QGDSLGNYYAT | 382 | GTNNRPS | 413 | NSRDSSGHHLL | 444 |
| BCMA-24 | RASQSVSSAYLA | 383 | GASTRAT | 405 | QHYGSSFNGSSLFT | 445 |
| BCMA-25 | RASQSVSNSLA | 384 | DASSRAT | 414 | QQFGTSSGLT | 446 |
| BCMA-26 | RASQSVSSSFLA | 385 | GASSRAT | 415 | QQYHSSPSWT | 425 |
| BCMA-27 | RASQSVSTTFLA | 386 | GSSNRAT | 416 | QQYHSSPSWT | 425 |
| BCMA-28 | RASQSISSYLN | 363 | AASSLQS | 395 | QQSYSTPYS | 447 |
| BCMA-29 | RATQSIGSSFLA | 387 | GASQRAT | 417 | QHYESSPSWT | 448 |
| BCMA-30 | RASQSVSSSYLA | 388 | GASSRAT | 415 | QQYGSPPRFT | 449 |
| BCMA-31 | RASQSVSSSYLA | 388 | GASSRAT | 415 | QHYGSSPSWT | 450 |
| BCMA-32 | RASQRVASNYLA | 389 | GASSRAT | 415 | QHYDSSPSWT | 451 |
| BCMA-33 | RASQSLSSNFLA | 390 | GASNWAT | 418 | QYYGTSPMYT | 452 |
| BCMA-34 | RSSQSLLHSNGYNYLD | 365 | LGSNRAS | 397 | MQALQTPLT | 453 |
| BCMA-35 | RASQSVSSSYLA | 388 | GTSSRAT | 419 | QHYGNSPPKFT | 454 |
| BCMA-36 | RASQSVASSFLA | 391 | GASGRAT | 420 | QHYGGSPRLT | 455 |
| BCMA-37 | RASQDVNTAVS | 392 | SASYRYT | 421 | QQHYSTPWT | 456 |
| BCMA-38 | RASESVSVIGAHLIH | 393 | LASNLET | 422 | LQSRIFPRT | 457 |
| BCMA-39 | RASESVTILGSHLIY | 394 | LASNVQT | 423 | LQSRTIPRT | 458 |
| BCMA-40 | RASESVTILGSHLIY | 394 | LASNVQT | 423 | LQSRTIPRT | 458 |

TABLE 11E
BCMA Binders - Heavy chain CDR sequences according to Kabat numbering scheme

| | | SEQ ID | | SEQ ID | | SEQ ID |
|---|---|---|---|---|---|---|
| Antibody | CDR-H1 | NO: | CDR-H2 | NO: | CDR-H3 | NO: |
| BCMA-1 | NHGMS | 537 | GIVYSGSTYYAASVKG | 556 | HGGESDV | 578 |
| BCMA-2 | NYAMS | 538 | GISRSGENTYYADSVKG | 557 | SPAHYYGGMDV | 579 |
| BCMA-3 | DYAMH | 539 | GISWNSGSIGYADSVKG | 558 | HSFLAY | 580 |
| BCMA-4 | NHGMS | 537 | GIVYSGSTYYAASVKG | 556 | HGGESDV | 578 |
| BCMA-5 | NFGIN | 540 | WINPKNNNTNYAQKFQG | 559 | GPYYYQSYMDV | 581 |
| BCMA-6 | SDAMT | 541 | VISGSGGTTYYADSVKG | 560 | LDSSGYYYARGPRY | 582 |
| BCMA-7 | NYGIT | 542 | WISAYNGNTNYAQKFQG | 561 | GPYYYYMDV | 583 |
| BCMA-8 | NHGMS | 537 | GIVYSGSTYYAASVKG | 556 | HGGESDV | 578 |
| BCMA-9 | NHGMS | 537 | GIVYSGSTYYAASVKG | 556 | HGGESDV | 578 |
| BCMA-10 | NHGMS | 537 | GIVYSGSTYYAASVKG | 556 | HGGESDV | 578 |
| BCMA-11 | DYYMS | 543 | YISSSGSTIYYADSVKG | 562 | ESGDGMDV | 584 |
| BCMA-12 | DYYMS | 543 | YISSSGNTIYYADSVKG | 563 | STMVREDY | 585 |
| BCMA-13 | NHGMS | 537 | GIVYSGSTYYAASVKG | 556 | HGGESDV | 578 |
| BCMA-14 | NHGMS | 537 | GIVYSGSTYYAASVKG | 556 | HGGESDV | 578 |
| BCMA-15 | NHGMS | 537 | GIVYSGSTYYAASVKG | 556 | HGGESDV | 578 |
| BCMA-16 | SSYYYWG | 544 | SIYYSGSAYYNPSLKS | 564 | HWQEWPDAFDI | 586 |
| BCMA-17 | TSGMCVS | 545 | RIDWDEDKFYSTSLKT | 565 | SGAGGTSATAFDI | 587 |
| BCMA-18 | SYSMN | 546 | SISSSSSYIYYADSVKG | 566 | TIAAVYAFDI | 588 |
| BCMA-19 | DYYMS | 543 | YISSSGSTIYYADSVKG | 562 | DLRGAFDI | 589 |
| BCMA-20 | SHYIH | 547 | MINPSGGVTAYSQTLQG | 567 | EGSGSGWYFDF | 590 |
| BCMA-21 | SGGYYWS | 548 | YIYYSGSTYYNPSLKS | 568 | AGIAARLRGAFDI | 591 |
| BCMA-22 | SYAIS | 549 | GIIPIFGTANYAQKFQG | 569 | RGGYQLLRWDVGLLRSAFDI | 592 |
| BCMA-23 | SNSAAWN | 550 | RTYYRSKWYSFYAISLKS | 570 | SSPEGLFLYWFDP | 593 |
| BCMA-24 | SYAMS | 551 | AISGSGGSTYYADSVKG | 571 | VEGSGSLDY | 594 |
| BCMA-25 | RYPMS | 552 | GISDSGVSTYYADSAKG | 572 | RAGSEASDI | 595 |
| BCMA-26 | SYAMS | 551 | AISGSGGSTYYADSVKG | 571 | ATYKRELRYYYGMDV | 596 |
| BCMA-27 | SYAMS | 551 | AISGSGGSTYYADSVKG | 571 | ATYKRELRYYYGMDV | 596 |
| BCMA-28 | DYAMH | 539 | GISWNSGSIGYADSVKG | 558 | VGKAVPDV | 597 |
| BCMA-29 | DYAMH | 539 | SINWKGNSLAYGDSVKG | 573 | HQGVAYYNYAMDV | 598 |
| BCMA-30 | SYAMS | 551 | AISGSGGSTYYADSVKG | 571 | VVRDGMDV | 599 |
| BCMA-31 | SYAMS | 551 | AISGSGGSTYYADSVKG | 571 | IPQTGTFDY | 600 |
| BCMA-32 | SYAMS | 551 | AISGSGGSTYYADSVKG | 571 | ANYKRELRYYYGMDV | 601 |
| BCMA-33 | SYAMS | 551 | AISGSGGSTYYADSVKG | 571 | ALVGATGAFDI | 602 |
| BCMA-34 | SYAMS | 551 | AISGSGGSTYYADSVKG | 571 | WFGEGFDP | 603 |
| BCMA-35 | SYAMS | 551 | AISGSGGSTYYADSVKG | 571 | VGYDSSGYYRDYYGMDV | 604 |
| BCMA-36 | SYAMS | 551 | AISGSGGSTYYADSVKG | 571 | MGWSSGYLGAFDI | 605 |
| BCMA-37 | NFGMN | 553 | WINTYTGESYFADDFKG | 574 | GEIYYGYDGGFAY | 606 |
| BCMA-38 | DYSIN | 554 | WINTETREPAYAYDFRG | 575 | DYSYAMDY | 607 |
| BCMA-39 | HYSMN | 555 | RINTESGVPIYADDFKG | 576 | DYLYSLDF | 608 |
| BCMA-40 | HYSMN | 555 | RINTETGEPLYADDFKG | 577 | DYLYSCDY | 609 |

TABLE 11F
BCMA Binders - Heavy chain CDR sequences according
to Chothia numbering scheme

| | | SEQ | | SEQ | | SEQ |
|---|---|---|---|---|---|---|
| | | ID | | ID | | ID |
| Antibody | CDR-H1 | NO: | CDR-H2 | NO: | CDR-H3 | NO: |
| BCMA-1 | GFALSNH | 610 | VYSGS | 630 | HGGESDV | 578 |
| BCMA-2 | GFTFSNY | 611 | SRSGEN | 631 | SPAHYYGGMDV | 579 |
| BCMA-3 | GFTFDDY | 612 | SWNSGS | 632 | HSFLAY | 580 |
| BCMA-4 | GFALSNH | 610 | VYSGS | 630 | HGGESDV | 578 |
| BCMA-5 | GYIFDNF | 613 | NPKNNN | 633 | GPYYYQSYMDV | 581 |
| BCMA-6 | GFTFSSD | 614 | SGSGGT | 634 | LDSSGYYYARGPRY | 582 |
| BCMA-7 | GYTFSNY | 615 | SAYNGN | 635 | GPYYYYMDV | 583 |
| BCMA-8 | GFALSNH | 610 | VYSGS | 630 | HGGESDV | 578 |
| BCMA-9 | GFALSNH | 610 | VYSGS | 630 | HGGESDV | 578 |
| BCMA-10 | GFALSNH | 610 | VYSGS | 630 | HGGESDV | 578 |
| BCMA-11 | GFTFSDY | 616 | SSSGST | 636 | ESGDGMDV | 584 |
| BCMA-12 | GFTFSDY | 616 | SSSGNT | 637 | STMVREDY | 585 |
| BCMA-13 | GFALSNH | 610 | VYSGS | 630 | HGGESDV | 578 |
| BCMA-14 | GFALSNH | 610 | VYSGS | 630 | HGGESDV | 578 |
| BCMA-15 | GFALSNH | 610 | VYSGS | 630 | HGGESDV | 578 |
| BCMA-16 | GGSISSS | 617 | YYSGS | 638 | HWQEWPDAFDI | 586 |
| | YY | | | | | |
| BCMA-17 | GFSLRTS | 618 | DWDED | 639 | SGAGGTSATAFDI | 587 |
| | GM | | | | | |
| BCMA-18 | GFTFSSY | 619 | SSSSSY | 640 | TIAAVYAFDI | 588 |
| BCMA-19 | GFTFSDY | 616 | SSSGST | 636 | DLRGAFDI | 589 |
| BCMA-20 | GYTVTSH | 620 | NPSGGV | 641 | EGSGSGWYFDF | 590 |
| BCMA-21 | GGSISSG | 621 | YYSGS | 638 | AGIAARLRGAFDI | 591 |
| | GY | | | | | |
| BCMA-22 | GGTFSSY | 622 | IPIFGT | 642 | RGGYQLLRWDVGLLRS | 592 |
| | | | | | AFDI | |
| BCMA-23 | GDSVSSN | 623 | YYRSKWY | 643 | SSPEGLFLYWFDP | 593 |
| | SA | | | | | |
| BCMA-24 | GFTFSSY | 619 | SGSGGS | 644 | VEGSGSLDY | 594 |
| BCMA-25 | GITFSRY | 624 | SDSGVS | 645 | RAGSEASDI | 595 |
| BCMA-26 | GFTFSSY | 619 | SGSGGS | 644 | ATYKRELRYYYGMDV | 596 |
| BCMA-27 | GFTFSSY | 619 | SGSGGS | 644 | ATYKRELRYYYGMDV | 596 |
| BCMA-28 | GFTFDDY | 612 | SWNSGS | 632 | VGKAVPDV | 597 |
| BCMA-29 | GFTFDDY | 612 | NWKGNS | 646 | HQGVAYYNYAMDV | 598 |
| BCMA-30 | GFTFSSY | 619 | SGSGGS | 644 | VVRDGMDV | 599 |
| BCMA-31 | GFTFSSY | 619 | SGSGGS | 644 | IPQTGTFDY | 600 |
| BCMA-32 | GFTFSSY | 619 | SGSGGS | 644 | ANYKRELRYYYGMDV | 601 |
| BCMA-33 | GFSFSSY | 625 | SGSGGS | 644 | ALVGATGAFDI | 602 |
| BCMA-34 | GFTFSSY | 619 | SGSGGS | 644 | WFGEGFDP | 603 |
| BCMA-35 | GFTFSSY | 619 | SGSGGS | 644 | VGYDSSGYYRDYYGMD | 604 |
| | | | | | V | |
| BCMA-36 | GFTFSSY | 619 | SGSGGS | 644 | MGWSSGYLGAFDI | 605 |
| BCMA-37 | GYTFTNF | 626 | NTYTGE | 647 | GEIYYGYDGGFAY | 606 |
| BCMA-38 | GYTFTDY | 627 | NTETRE | 648 | DYSYAMDY | 607 |
| BCMA-39 | GYTFRHY | 628 | NTESGV | 649 | DYLYSLDF | 608 |
| BCMA-40 | GYTFTHY | 629 | NTETGE | 650 | DYLYSCDY | 609 |

TABLE 11G
BCMA Binders - Heavy chain CDR sequences according to combination of Kabat and
Chothia numbering schemes

| | | SEQ | | SEQ | | SEQ |
|---|---|---|---|---|---|---|
| Antibody | CDR-H1 | ID NO: | CDR-H2 | ID NO: | CDR-H3 | ID NO: |
| BCMA-1 | GFALSNHG | 651 | GIVYSGSTYYAAS | 556 | HGGESDV | 578 |
| | MS | | VKG | | | |
| BCMA-2 | GFTFSNYA | 652 | GISRSGENTYYAD | 557 | SPAHYYGGMDV | 579 |
| | MS | | SVKG | | | |
| BCMA-3 | GFTFDDYA | 653 | GISWNSGSIGYAD | 558 | HSFLAY | 580 |
| | MH | | SVKG | | | |
| BCMA-4 | GFALSNHG | 651 | GIVYSGSTYYAAS | 556 | HGGESDV | 578 |
| | MS | | VKG | | | |
| BCMA-5 | GYIFDNFGI | 654 | WINPKNNNTNYA | 559 | GPYYYQSYMDV | 581 |
| | N | | QKFQG | | | |
| BCMA-6 | GFTFSSDA | 655 | VISGSGGTTYYAD | 560 | LDSSGYYYARGPR | 582 |
| | MT | | SVKG | | Y | |
| BCMA-7 | GYTFSNYG | 656 | WISAYNGNTNYA | 561 | GPYYYYMDV | 583 |
| | IT | | QKFQG | | | |
| BCMA-8 | GFALSNHG | 651 | GIVYSGSTYYAAS | 556 | HGGESDV | 578 |
| | MS | | VKG | | | |
| BCMA-9 | GFALSNHG | 651 | GIVYSGSTYYAAS | 556 | HGGESDV | 578 |
| | MS | | VKG | | | |
| BCMA-10 | GFALSNHG | 651 | GIVYSGSTYYAAS | 556 | HGGESDV | 578 |
| | MS | | VKG | | | |
| BCMA-11 | GFTFSDYY | 657 | YISSSGSTIYYADS | 562 | ESGDGMDV | 584 |
| | MS | | VKG | | | |
| BCMA-12 | GFTFSDYY | 657 | YISSSGNTIYYAD | 563 | STMVREDY | 585 |
| | MS | | SVKG | | | |
| BCMA-13 | GFALSNHG | 651 | GIVYSGSTYYAAS | 556 | HGGESDV | 578 |
| | MS | | VKG | | | |
| BCMA-14 | GFALSNHG | 651 | GIVYSGSTYYAAS | 556 | HGGESDV | 578 |
| | MS | | VKG | | | |
| BCMA-15 | GFALSNHG | 651 | GIVYSGSTYYAAS | 556 | HGGESDV | 578 |
| | MS | | VKG | | | |
| BCMA-16 | GGSISSSY | 658 | SIYYSGSAYYNPS | 564 | HWQEWPDAFDI | 586 |
| | YYWG | | LKS | | | |
| BCMA-17 | GFSLRTSG | 659 | RIDWDEDKFYSTS | 565 | SGAGGTSATAFDI | 587 |
| | MCVS | | LKT | | | |
| BCMA-18 | GFTFSSYS | 660 | SISSSSSYIYYADS | 566 | TIAAVYAFDI | 588 |
| | MN | | VKG | | | |
| BCMA-19 | GFTFSDYY | 657 | YISSSGSTIYYADS | 562 | DLRGAFDI | 589 |
| | MS | | VKG | | | |
| BCMA-20 | GYTVTSHYI | 661 | MINPSGGVTAYS | 567 | EGSGSGWYFDF | 590 |
| | H | | QTLQG | | | |
| BCMA-21 | GGSISSGG | 662 | YIYYSGSTYYNPS | 568 | AGIAARLRGAFDI | 591 |
| | YYWS | | LKS | | | |
| BCMA-22 | GGTFSSYAI | 663 | GIIPIFGTANYAQK | 569 | RGGYQLLRWDVGL | 592 |
| | S | | FQG | | LRSAFDI | |
| BCMA-23 | GDSVSSNS | 664 | RTYYRSKWYSFY | 570 | SSPEGLFLYWFDP | 593 |
| | AAWN | | AISLKS | | | |
| BCMA-24 | GFTFSSYA | 665 | AISGSGGSTYYAD | 571 | VEGSGSLDY | 594 |
| | MS | | SVKG | | | |
| BCMA-25 | GITFSRYP | 666 | GISDSGVSTYYAD | 572 | RAGSEASDI | 595 |
| | MS | | SAKG | | | |
| BCMA-26 | GFTFSSYA | 665 | AISGSGGSTYYAD | 571 | ATYKRELRYYYGM | 596 |
| | MS | | SVKG | | DV | |
| BCMA-27 | GFTFSSYA | 665 | AISGSGGSTYYAD | 571 | ATYKRELRYYYGM | 596 |
| | MS | | SVKG | | DV | |
| BCMA-28 | GFTFDDYA | 653 | GISWNSGSIGYAD | 558 | VGKAVPDV | 597 |
| | MH | | SVKG | | | |
| BCMA-29 | GFTFDDYA | 653 | SINWKGNSLAYG | 573 | HQGVAYYNYAMDV | 598 |
| | MH | | DSVKG | | | |
| BCMA-30 | GFTFSSYA | 665 | AISGSGGSTYYAD | 571 | VVRDGMDV | 599 |
| | MS | | SVKG | | | |
| BCMA-31 | GFTFSSYA | 665 | AISGSGGSTYYAD | 571 | IPQTGTFDY | 600 |
| | MS | | SVKG | | | |
| BCMA-32 | GFTFSSYA | 665 | AISGSGGSTYYAD | 571 | ANYKRELRYYYGM | 601 |
| | MS | | SVKG | | DV | |
| BCMA-33 | GFSFSSYA | 667 | AISGSGGSTYYAD | 571 | ALVGATGAFDI | 602 |
| | MS | | SVKG | | | |
| BCMA-34 | GFTFSSYA | 665 | AISGSGGSTYYAD | 571 | WFGEGFDP | 603 |
| | MS | | SVKG | | | |
| BCMA-35 | GFTFSSYA | 665 | AISGSGGSTYYAD | 571 | VGYDSSGYYRDYY | 604 |
| | MS | | SVKG | | GMDV | |
| BCMA-36 | GFTFSSYA | 665 | AISGSGGSTYYAD | 571 | MGWSSGYLGAFDI | 605 |
| | MS | | SVKG | | | |
| BCMA-37 | GYTFTNFG | 668 | WINTYTGESYFAD | 574 | GEIYYGYDGGFAY | 606 |
| | MN | | DFKG | | | |
| BCMA-38 | GYTFTDYSI | 669 | WINTETREPAYAY | 575 | DYSYAMDY | 607 |
| | N | | DFRG | | | |
| BCMA-39 | GYTFRHYS | 670 | RINTESGVPIYAD | 576 | DYLYSLDF | 608 |
| | MN | | DFKG | | | |
| BCMA-40 | GYTFTHYS | 671 | RINTETGEPLYAD | 577 | DYLYSCDY | 609 |
| | MN | | DFKG | | | |

In some embodiments, the ABM comprises the CDR sequences of BCMA-1. In some embodiments, the ABM comprises the CDR sequences of BCMA-2. In some embodiments, the ABM comprises the CDR sequences of BCMA-3. In some embodiments, the ABM comprises the CDR sequences of BCMA-4. In some embodiments, the ABM comprises the CDR sequences of BCMA-5. In some embodiments, the ABM comprises the CDR sequences of BCMA-6. In some embodiments, the ABM comprises the CDR sequences of BCMA-7. In some embodiments, the ABM comprises the CDR sequences of BCMA-8. In some embodiments, the ABM comprises the CDR sequences of BCMA-9. In some embodiments, the ABM comprises the CDR sequences of BCMA-10. In some embodiments, the ABM comprises the CDR sequences of BCMA-11. In some embodiments, the ABM comprises the CDR sequences of BCMA-12. In some embodiments, the ABM comprises the CDR sequences of BCMA-13. In some embodiments, the ABM comprises the CDR sequences of BCMA-14. In some embodiments, the ABM comprises the CDR sequences of BCMA-15. In some embodiments, the ABM comprises the CDR sequences of BCMA-16. In some embodiments, the ABM comprises the CDR sequences of BCMA-17. In some embodiments, the ABM comprises the CDR sequences of BCMA-18. In some embodiments, the ABM comprises the CDR sequences of BCMA-19. In some embodiments, the ABM comprises the CDR sequences of BCMA-20. In some embodiments, the ABM comprises the CDR sequences of BCMA-21. In some embodiments, the ABM comprises the CDR sequences of BCMA-22. In some embodiments, the ABM comprises the CDR sequences of BCMA-23. In some embodiments, the ABM comprises the CDR sequences of BCMA-24. In some embodiments, the ABM comprises the CDR sequences of BCMA-25. In some embodiments, the ABM comprises the CDR sequences of BCMA-26. In some embodiments, the ABM comprises the CDR sequences of BCMA-27. In some embodiments, the ABM comprises the CDR sequences of BCMA-28. In some embodiments, the ABM comprises the CDR sequences of BCMA-29. In some embodiments, the ABM comprises the CDR sequences of BCMA-30. In some embodiments, the ABM comprises the CDR sequences of BCMA-31. In some embodiments, the ABM comprises the CDR sequences of BCMA-32. In some embodiments, the ABM comprises the CDR sequences of BCMA-33. In some embodiments, the ABM comprises the CDR sequences of BCMA-34. In some embodiments, the ABM comprises the CDR sequences of BCMA-35. In some embodiments, the ABM comprises the CDR sequences of BCMA-36. In some embodiments, the ABM comprises the CDR sequences of BCMA-37. In some embodiments, the ABM comprises the CDR sequences of BCMA-38. In some embodiments, the ABM comprises the CDR sequences of BCMA-39. In some embodiments, the ABM comprises the CDR sequences of BCMA-40.

In some embodiments, the CDRs are defined by Kabat numbering, as set forth in Table 11B and 11E. In other embodiments, the CDRs are defined by Chothia numbering, as set forth in Table 11C and 11F. In yet other embodiments, the CDRs are defined by a combination of Kabat and Chothia numbering, as set forth in Table 11D and 11G.

In some embodiments, the MBMs (e.g., TBMs) comprising a ABM that binds to BCMA can comprise the heavy and light chain variable sequences of any of BCMA-1 to BCMA-40. In some embodiments, the ABM comprises the heavy and light chain variable sequences of BCMA-1, as set forth in Table 11A. In some embodiments, the ABM comprises the heavy and light chain variable sequences of BCMA-2, as set forth in Table 11A. In some embodiments, the ABM comprises the heavy and light chain variable sequences of BCMA-3, as set forth in Table 11A. In some embodiments, the ABM comprises the heavy and light chain variable sequences of BCMA-4, as set forth in Table 11A. In some embodiments, the ABM comprises the heavy and light chain variable sequences of BCMA-5, as set forth in Table 11A. In some embodiments, the ABM comprises the heavy and light chain variable sequences of BCMA-6, as set forth in Table 11A. In some embodiments, the ABM comprises the heavy and light chain variable sequences of BCMA-7, as set forth in Table 11A. In some embodiments, the ABM comprises the heavy and light chain variable sequences of BCMA-8, as set forth in Table 11A. In some embodiments, the ABM comprises the heavy and light chain variable sequences of BCMA-9, as set forth in Table 11A. In some embodiments, the ABM comprises the heavy and light chain variable sequences of BCMA-10, as set forth in Table 11A. In some embodiments, the ABM comprises the heavy and light chain variable sequences of BCMA-11, as set forth in Table 11A. In some embodiments, the ABM comprises the heavy and light chain variable sequences of BCMA-12, as set forth in Table 11A. In some embodiments, the ABM comprises the heavy and light chain variable sequences of BCMA-13, as set forth in Table 11A. In some embodiments, the ABM comprises the heavy and light chain variable sequences of BCMA-14, as set forth in Table 11A. In some embodiments, the ABM comprises the heavy and light chain variable sequences of BCMA-15, as set forth in Table 11A. In some embodiments, the ABM comprises the heavy and light chain variable sequences of BCMA-16, as set forth in Table 11A. In some embodiments, the ABM comprises the heavy and light chain variable sequences of BCMA-17, as set forth in Table 11A. In some embodiments, the ABM comprises the heavy and light chain variable sequences of BCMA-18, as set forth in Table 11A. In some embodiments, the ABM comprises the heavy and light chain variable sequences of BCMA-19, as set forth in Table 11A. In some embodiments, the ABM comprises the heavy and light chain variable sequences of BCMA-20, as set forth in Table 11A. In some embodiments, the ABM comprises the heavy and light chain variable sequences of BCMA-21, as set forth in Table 11A. In some embodiments, the ABM comprises the heavy and light chain variable sequences of BCMA-22, as set forth in Table 11A. In some embodiments, the ABM comprises the heavy and light chain variable sequences of BCMA-23, as set forth in Table 11A. In some embodiments, the ABM comprises the heavy and light chain variable sequences of BCMA-24, as set forth in Table 11A. In some embodiments, the ABM comprises the heavy and light chain variable sequences of BCMA-25, as set forth in Table 11A. In some embodiments, the ABM comprises the heavy and light chain variable sequences of BCMA-26, as set forth in Table 11A. In some embodiments, the ABM comprises the heavy and light chain variable sequences of BCMA-27, as set forth in Table 11A. In some embodiments, the ABM comprises the heavy and light chain variable sequences of BCMA-28, as set forth in Table 11A. In some embodiments, the ABM comprises the heavy and light chain variable sequences of BCMA-29, as set forth in Table 11A. In some embodiments, the ABM comprises the heavy and light chain variable sequences of BCMA-30, as set forth in Table 11A. In some embodiments, the ABM comprises the heavy and light chain variable sequences of BCMA-31, as set forth in Table 11A. In some embodiments, the ABM comprises the heavy and light chain variable sequences of BCMA-32, as set forth in Table 11A. In some embodiments, the ABM comprises the heavy and light chain variable sequences of BCMA-33, as set forth in Table 11A. In some embodiments, the ABM comprises the heavy and light chain variable sequences of BCMA-34, as set forth in Table 11A. In some embodiments, the ABM comprises the heavy and light chain variable sequences of BCMA-35, as set forth in Table 11A. In some embodiments, the ABM comprises the heavy and light chain variable sequences of BCMA-36, as set forth in Table 11A. In some embodiments, the ABM comprises the heavy and light chain variable sequences of BCMA-37, as set forth in Table 11A. In some embodiments, the ABM comprises the heavy and light chain variable sequences of BCMA-38, as set forth in Table 11A. In some embodiments, the ABM comprises the heavy and light chain variable sequences of BCMA-39, as set forth in Table 11A. In some embodiments, the ABM comprises the heavy and light chain variable sequences of BCMA-40, as set forth in Table 11A.

### 7.6.2. CD19

B cells express cell surface proteins which can be utilized as markers for differentiation and identification. One such human B-cell marker is a CD19 antigen and is found on mature B cells but not on plasma cells. CD19 is expressed during early pre-B cell development and remains until plasma cell differentiation. CD19 is expressed on both normal B cells and cancerous B cells whose abnormal growth can lead to B-cell lymphomas. For example, CD19 is expressed on B-cell lineage cancers, including, but not limited to non-Hodgkin's lymphoma (B-NHL), chronic lymphocytic leukemia, and acute lymphoblastic leukemia.

In certain aspects, a MBM (e.g., a TBM) of the disclosure comprises a TAA 1 ABM or TAA 2 ABM that specifically binds to CD19. Exemplary CDR and variable domain sequences that can be incorporated into a TAA 1 ABM or TAA 2 ABM that specifically binds to CD19 are set forth in Table 12 below.

TABLE 12
CD19 Binders

| | | | SEQ |
|---|---|---|---|
| Name | Domain | Sequence | ID NO: |
| CD19-H1 | CDR-H1 | DYGVS | 672 |
| CD19-H2A | CDR-H2 | VIWGSETTYYNSALKS | 673 |
| CD19-H2B | CDR-H2 | VIWGSETTYYSSSLKS | 674 |
| CD19-H2C | CDR-H2 | VIWGSETTYYQSSLKS | 675 |
| CD19-H2D | CDR-H2 | VIWGSETTYYNSSLKS | 676 |
| CD19-H3 | CDR-H3 | HYYYGGSYAMDY | 677 |
| CD19-L1 | CDR-L1 | RASQDISKYLN | 678 |
| CD19-L2 | CDR-L2 | HTSRLHS | 679 |
| CD19-L3 | CDR-L3 | QQGNTLPYT | 680 |
| CD19-VHA | VH | EVKLQESGPGLVAPSQSLSVTCTVSGVSLPDYGVSWI | 681 |
| | | RQPPRKGLEWLGVIWGSETTYYNSALKSRLTIIKDNSK | |
| | | SQVFLKMNSLQTDDTAIYYCAKHYYYGGSYAMDYWG | |
| | | QGTSVTVSS | |
| CD19-VHB | VH | QVQLQESGPGLVKPSETLSLTCTVSGVSLPDYGVSWIR | 682 |
| | | QPPGKGLEWIGVIWGSETTYYSSSLKSRVTISKDNSKN | |
| | | QVSLKLSSVTAADTAVYYCAKHYYYGGSYAMDYWGQ | |
| | | GTLVTVSS | |
| CD19-VHC | VH | QVQLQESGPGLVKPSETLSLTCTVSGVSLPDYGVSWIR | 683 |
| | | QPPGKGLEWIGVIWGSETTYYQSSLKSRVTISKDNSKN | |
| | | QVSLKLSSVTAADTAVYYCAKHYYYGGSYAMDYWGQ | |
| | | GTLVTVSS | |
| CD19-VHD | VH | QVQLQESGPGLVKPSETLSLTCTVSGVSLPDYGVSWIR | 684 |
| | | QPPGKGLEWIGVIWGSETTYYNSSLKSRVTISKDNSKN | |
| | | QVSLKLSSVTAADTAVYYCAKHYYYGGSYAMDYWGQ | |
| | | GTLVTVSS | |
| CD19-VLA | VL | DIQMTQTTSSLSASLGDRVTISCRASQDISKYLNWYQQ | 685 |
| | | KPDGTVKLLIYHTSRLHSGVPSRFSGSGSGTDYSLTISN | |
| | | LEQEDIATYFCQQGNTLPYTFGGGTKLEIT | |
| CD19-VLB | VL | EIVMTQSPATLSLSPGERATLSCRASQDISKYLNWYQQ | 686 |
| | | KPGQAPRLLIYHTSRLHSGIPARFSGSGSGTDYTLTISS | |
| | | LQPEDFAVYFCQQGNTLPYTFGQGTKLEIK | |
| CD19-scFv1 | scFv | EIVMTQSPATLSLSPGERATLSCRASQDISKYLNWYQQ | 687 |
| | | KPGQAPRLLIYHTSRLHSGIPARFSGSGSGTDYTLTISS | |
| | | LQPEDFAVYFCQQGNTLPYTFGQGTKLEIKGGGGSGG | |
| | | GGSGGGGSQVQLQESGPGLVKPSETLSLTCTVSGVSL | |
| | | PDYGVSWIRQPPGKGLEWIGVIWGSETTYYSSSLKSR | |
| | | VTISKDNSKNQVSLKLSSVTAADTAVYYCAKHYYYGGS | |
| | | YAMDYWGQGTLVTVSS | |
| CD19-scFv2 | scFv | EIVMTQSPATLSLSPGERATLSCRASQDISKYLNWYQQ | 688 |
| | | KPGQAPRLLIYHTSRLHSGIPARFSGSGSGTDYTLTISS | |
| | | LQPEDFAVYFCQQGNTLPYTFGQGTKLEIKGGGGSGG | |
| | | GGSGGGGSQVQLQESGPGLVKPSETLSLTCTVSGVSL | |
| | | PDYGVSWIRQPPGKGLEWIGVIWGSETTYYQSSLKSR | |
| | | VTISKDNSKNQVSLKLSSVTAADTAVYYCAKHYYYGGS | |
| | | YAMDYWGQGTLVTVSS | |
| CD19-scFv3 | scFv | QVQLQESGPGLVKPSETLSLTCTVSGVSLPDYGVSWIR | 689 |
| | | QPPGKGLEWIGVIWGSETTYYSSSLKSRVTISKDNSKN | |
| | | QVSLKLSSVTAADTAVYYCAKHYYYGGSYAMDYWGQ | |
| | | GTLVTVSSGGGGSGGGGSGGGGSEIVMTQSPATLSLS | |
| | | PGERATLSCRASQDISKYLNWYQQKPGQAPRLLIYHTS | |
| | | RLHSGIPARFSGSGSGTDYTLTISSLQPEDFAVYFCQQ | |
| | | GNTLPYTFGQGTKLEIK | |
| CD19-scFv4 | scFv | QVQLQESGPGLVKPSETLSLTCTVSGVSLPDYGVSWIR | 690 |
| | | QPPGKGLEWIGVIWGSETTYYQSSLKSRVTISKDNSKN | |
| | | QVSLKLSSVTAADTAVYYCAKHYYYGGSYAMDYWGQ | |
| | | GTLVTVSSGGGGSGGGGSGGGGSEIVMTQSPATLSLS | |
| | | PGERATLSCRASQDISKYLNWYQQKPGQAPRLLIYHTS | |
| | | RLHSGIPARFSGSGSGTDYTLTISSLQPEDFAVYFCQQ | |
| | | GNTLPYTFGQGTKLEIK | |
| CD19-scFv5 | scFv | EIVMTQSPATLSLSPGERATLSCRASQDISKYLNWYQQ | 691 |
| | | KPGQAPRLLIYHTSRLHSGIPARFSGSGSGTDYTLTISS | |
| | | LQPEDFAVYFCQQGNTLPYTFGQGTKLEIKGGGGSGG | |
| | | GGSGGGGSGGGGSQVQLQESGPGLVKPSETLSLTCT | |
| | | VSGVSLPDYGVSWIRQPPGKGLEWIGVIWGSETTYYS | |
| | | SSLKSRVTISKDNSKNQVSLKLSSVTAADTAVYYCAKH | |
| | | YYYGGSYAMDYWGQGTLVTVSS | |
| CD19-scFv6 | scFv | EIVMTQSPATLSLSPGERATLSCRASQDISKYLNWYQQ | 692 |
| | | KPGQAPRLLIYHTSRLHSGIPARFSGSGSGTDYTLTISS | |
| | | LQPEDFAVYFCQQGNTLPYTFGQGTKLEIKGGGGSGG | |
| | | GGSGGGGSGGGGSQVQLQESGPGLVKPSETLSLTCT | |
| | | VSGVSLPDYGVSWIRQPPGKGLEWIGVIWGSETTYYQ | |
| | | SSLKSRVTISKDNSKNQVSLKLSSVTAADTAVYYCAKH | |
| | | YYYGGSYAMDYWGQGTLVTVSS | |
| CD19-scFv7 | scFv | QVQLQESGPGLVKPSETLSLTCTVSGVSLPDYGVSWIR | 693 |
| | | QPPGKGLEWIGVIWGSETTYYSSSLKSRVTISKDNSKN | |
| | | QVSLKLSSVTAADTAVYYCAKHYYYGGSYAMDYWGQ | |
| | | GTLVTVSSGGGGSGGGGSGGGGSGGGGSEIVMTQS | |
| | | PATLSLSPGERATLSCRASQDISKYLNWYQQKPGQAP | |
| | | RLLIYHTSRLHSGIPARFSGSGSGTDYTLTISSLQPEDF | |
| | | AVYFCQQGNTLPYTFGQGTKLEIK | |
| CD19-scFv8 | scFv | QVQLQESGPGLVKPSETLSLTCTVSGVSLPDYGVSWIR | 694 |
| | | QPPGKGLEWIGVIWGSETTYYQSSLKSRVTISKDNSKN | |
| | | QVSLKLSSVTAADTAVYYCAKHYYYGGSYAMDYWGQ | |
| | | GTLVTVSSGGGGSGGGGSGGGGSGGGGSEIVMTQS | |
| | | PATLSLSPGERATLSCRASQDISKYLNWYQQKPGQAP | |
| | | RLLIYHTSRLHSGIPARFSGSGSGTDYTLTISSLQPEDF | |
| | | AVYFCQQGNTLPYTFGQGTKLEIK | |
| CD19-scFv9 | scFv | EIVMTQSPATLSLSPGERATLSCRASQDISKYLNWYQQ | 695 |
| | | KPGQAPRLLIYHTSRLHSGIPARFSGSGSGTDYTLTISS | |
| | | LQPEDFAVYFCQQGNTLPYTFGQGTKLEIKGGGGSGG | |
| | | GGSGGGGSGGGGSQVQLQESGPGLVKPSETLSLTCT | |
| | | VSGVSLPDYGVSWIRQPPGKGLEWIGVIWGSETTYYN | |
| | | SSLKSRVTISKDNSKNQVSLKLSSVTAADTAVYYCAKH | |
| | | YYYGGSYAMDYWGQGTLVTVSS | |
| CD19- | scFv | QVQLQESGPGLVKPSETLSLTCTVSGVSLPDYGVSWIR | 696 |
| scFv10 | | QPPGKGLEWIGVIWGSETTYYNSSLKSRVTISKDNSKN | |
| | | QVSLKLSSVTAADTAVYYCAKHYYYGGSYAMDYWGQ | |
| | | GTLVTVSSGGGGSGGGGSGGGGSGGGGSEIVMTQS | |
| | | PATLSLSPGERATLSCRASQDISKYLNWYQQKPGQAP | |
| | | RLLIYHTSRLHSGIPARFSGSGSGTDYTLTISSLQPEDF | |
| | | AVYFCQQGNTLPYTFGQGTKLEIK | |
| CD19- | scFv | EIVMTQSPATLSLSPGERATLSCRASQDISKYLNWYQQ | 697 |
| scFv11 | | KPGQAPRLLIYHTSRLHSGIPARFSGSGSGTDYTLTISS | |
| | | LQPEDFAVYFCQQGNTLPYTFGQGTKLEIKGGGGSGG | |
| | | GGSGGGGSQVQLQESGPGLVKPSETLSLTCTVSGVSL | |
| | | PDYGVSWIRQPPGKGLEWIGVIWGSETTYYNSSLKSR | |
| | | VTISKDNSKNQVSLKLSSVTAADTAVYYCAKHYYYGGS | |
| | | YAMDYWGQGTLVTVSS | |
| CD19- | scFv | QVQLQESGPGLVKPSETLSLTCTVSGVSLPDYGVSWIR | 698 |
| scFv12 | | QPPGKGLEWIGVIWGSETTYYNSSLKSRVTISKDNSKN | |
| | | QVSLKLSSVTAADTAVYYCAKHYYYGGSYAMDYWGQ | |
| | | GTLVTVSSGGGGSGGGGSGGGGSEIVMTQSPATLSLS | |
| | | PGERATLSCRASQDISKYLNWYQQKPGQAPRLLIYHTS | |
| | | RLHSGIPARFSGSGSGTDYTLTISSLQPEDFAVYFCQQ | |
| | | GNTLPYTFGQGTKLEIK | |

In certain aspects, the ABM comprises heavy chain CDRs having the amino acid sequences of CD19-H1, CD19-H2A, and CD19-H3 as set forth in Table 12 and light chain CDRs having the amino acid sequences of CD19-L1, CD19-L2, and CD19-L3 as set forth in Table 12. In a specific embodiment, the ABM comprises a heavy chain variable region having the amino acid sequences of VHA as set forth in Table 12 and a light chain variable region having the amino acid sequences of VLA as set forth in Table 12.

In other aspects, the ABM comprises heavy chain CDRs having the amino acid sequences of CD19-H1, CD19-H2B, and CD19-H3 as set forth in Table 12 and light chain CDRs having the amino acid sequences of CD19-L1, CD19-L2, and CD19-L3 as set forth in Table 12. In a specific embodiment, the ABM comprises a heavy chain variable region having the amino acid sequences of VHB as set forth in Table 12 and a light chain variable region having the amino acid sequences of VLB as set forth in Table 12.

In further aspects, the ABM comprises heavy chain CDRs having the amino acid sequences of CD19-H1, CD19-H2C, and CD19-H3 as set forth in Table 12 and light chain CDRs having the amino acid sequences of CD19-L1, CD19-L2, and CD19-L3 as set forth in Table 12. In a specific embodiment, ABM comprises a heavy chain variable region having the amino acid sequences of VHC as set forth in Table 12 and a light chain variable region having the amino acid sequences of VLB as set forth in Table 12.

In further aspects, the ABM comprises heavy chain CDRs having the amino acid sequences of CD19-H1, CD19-H2D, and CD19-H3 as set forth in Table 12 and light chain CDRs having the amino acid sequences of CD19-L1, CD19-L2, and CD19-L3 as set forth in Table 12. In a specific embodiment, the ABM comprises a heavy chain variable region having the amino acid sequences of VHD as set forth in Table 12 and a light chain variable region having the amino acid sequences of VLB as set forth in Table 12.

In yet further aspects, the ABM is in the form of an scFV. Exemplary anti-CD19 scFvs comprise the amino acid sequence of any one of CD19-scFv1 through CD19-scFv12 as set forth in Table 12.

### 7.7. Nucleic Acids and Host Cells

In another aspect, the disclosure provides nucleic acids encoding the MBMs (e.g., TBMs) of the disclosure. In some embodiments, the MBMs are encoded by a single nucleic acid. In other embodiments, the MBMs are encoded by a plurality (e.g., two, three, four or more) nucleic acids.

A single nucleic acid can encode a MBM that comprises a single polypeptide chain, a MBM that comprises two or more polypeptide chains, or a portion of a MBM that comprises more than two polypeptide chains (for example, a single nucleic acid can encode two polypeptide chains of a TBM comprising three, four or more polypeptide chains, or three polypeptide chains of a TBM comprising four or more polypeptide chains). For separate control of expression, the open reading frames encoding two or more polypeptide chains can be under the control of separate transcriptional regulatory elements (e.g., promoters and/or enhancers). The open reading frames encoding two or more polypeptides can also be controlled by the same transcriptional regulatory elements, and separated by internal ribosome entry site (IRES) sequences allowing for translation into separate polypeptides.

In some embodiments, a MBM comprising two or more polypeptide chains is encoded by two or more nucleic acids. The number of nucleic acids encoding a MBM can be equal to or less than the number of polypeptide chains in the MBM (for example, when more than one polypeptide chains are encoded by a single nucleic acid).

The nucleic acids of the disclosure can be DNA or RNA (e.g., mRNA).

In another aspect, the disclosure provides host cells and vectors containing the nucleic acids of the disclosure. The nucleic acids may be present in a single vector or separate vectors present in the same host cell or separate host cell, as described in more detail herein below.

### 7.7.1. Vectors

The disclosure provides vectors comprising nucleotide sequences encoding a MBM (e.g., a TBM) or a MBM component described herein. In one embodiment, the vectors comprise nucleotides encoding an immunoglobulin-based ABM described herein. In one embodiment, the vectors comprise nucleotides encoding an Fc domain described herein. In one embodiment, the vectors comprise nucleotides encoding a recombinant non-immunoglobulin based ABM described herein. A vector of the disclosure can encode one or more ABMs, one or more Fc domains, one or more non-immunoglobulin based ABM, or a combination thereof (e.g., when multiple components or sub-components are encoded as a single polypeptide chain). In one embodiment, the vectors comprise the nucleotide sequences described herein. The vectors include, but are not limited to, a virus, plasmid, cosmid, lambda phage or a yeast artificial chromosome (YAC).

Numerous vector systems can be employed. For example, one class of vectors utilizes DNA elements which are derived from animal viruses such as, for example, bovine papilloma virus, polyoma virus, adenovirus, vaccinia virus, baculovirus, retroviruses (Rous Sarcoma Virus, MMTV or MOMLV) or SV40 virus. Another class of vectors utilizes RNA elements derived from RNA viruses such as Semliki Forest virus, Eastern Equine Encephalitis virus and Flaviviruses.

Additionally, cells which have stably integrated the DNA into their chromosomes may be selected by introducing one or more markers which allow for the selection of transfected host cells. The marker may provide, for example, prototropy to an auxotrophic host, biocide resistance (e.g., antibiotics), or resistance to heavy metals such as copper, or the like. The selectable marker gene can be either directly linked to the DNA sequences to be expressed, or introduced into the same cell by cotransformation. Additional elements may also be needed for optimal synthesis of mRNA. These elements may include splice signals, as well as transcriptional promoters, enhancers, and termination signals.

Once the expression vector or DNA sequence containing the constructs has been prepared for expression, the expression vectors may be transfected or introduced into an appropriate host cell. Various techniques may be employed to achieve this, such as, for example, protoplast fusion, calcium phosphate precipitation, electroporation, retroviral transduction, viral transfection, gene gun, lipid based transfection or other conventional techniques. Methods and conditions for culturing the resulting transfected cells and for recovering the expressed polypeptides are known to those skilled in the art, and may be varied or optimized depending upon the specific expression vector and mammalian host cell employed, based upon the present description.

### 7.7.2. Cells

The disclosure also provides host cells comprising a nucleic acid of the disclosure.

In one embodiment, the host cells are genetically engineered to comprise one or more nucleic acids described herein.

In one embodiment, the host cells are genetically engineered by using an expression cassette. The phrase “expression cassette,” refers to nucleotide sequences, which are capable of affecting expression of a gene in hosts compatible with such sequences. Such cassettes may include a promoter, an open reading frame with or without introns, and a termination signal. Additional factors necessary or helpful in effecting expression may also be used, such as, for example, an inducible promoter.

The disclosure also provides host cells comprising the vectors described herein.

The cell can be, but is not limited to, a eukaryotic cell, a bacterial cell, an insect cell, or a human cell. Suitable eukaryotic cells include, but are not limited to, Vero cells, HeLa cells, COS cells, CHO cells, HEK293 cells, BHK cells and MDCKII cells. Suitable insect cells include, but are not limited to, Sf9 cells.

### 7.8. Antibody-Drug Conjugates

The MBMs (e.g., TBMs) of the disclosure can be conjugated, e.g., via a linker, to a drug moiety. Such conjugates are referred to herein as antibody-drug conjugates (or “ADCs”) for convenience, notwithstanding the fact that one or more (or all) of the ABMs might be based on non-immunoglobulin scaffolds.

In certain aspects, the drug moiety exerts a cytotoxic or cytostatic activity. In one embodiment, the drug moiety is chosen from a maytansinoid, a kinesin-like protein KIF11 inhibitor, a V-ATPase (vacuolar-type H+-ATPase) inhibitor, a pro-apoptotic agent, a Bcl2 (B-cell lymphoma 2) inhibitor, an MCL1 (myeloid cell leukemia 1) inhibitor, a HSP90 (heat shock protein 90) inhibitor, an IAP (inhibitor of apoptosis) inhibitor, an mTOR (mechanistic target of rapamycin) inhibitor, a microtubule stabilizer, a microtubule destabilizer, an auristatin, a dolastatin, a MetAP (methionine aminopeptidase), a CRM1 (chromosomal maintenance 1) inhibitor, a DPPIV (dipeptidyl peptidase IV) inhibitor, a proteasome inhibitor, an inhibitor of a phosphoryl transfer reaction in mitochondria, a protein synthesis inhibitor, a kinase inhibitor, a CDK2 (cyclin-dependent kinase 2) inhibitor, a CDK9 (cyclin-dependent kinase 9) inhibitor, a kinesin inhibitor, an HDAC (histone deacetylase) inhibitor, a DNA damaging agent, a DNA alkylating agent, a DNA intercalator, a DNA minor groove binder, a RNA polymerase inhibitor, a topoisomerase inhibitor, or a DHFR (dihydrofolate reductase) inhibitor.

In one embodiment, the linker is chosen from a cleavable linker, a non-cleavable linker, a hydrophilic linker, a procharged linker, or a dicarboxylic acid based linker.

In specific embodiments, the ADCs are compounds according to structural formula (I):

[D-L-XY]ₙ-Ab

or salts thereof, where each “D” represents, independently of the others, a cytotoxic and/or cytostatic agent (“drug”); each “L” represents, independently of the others, a linker; “Ab” represents a MBM described herein; each “XY” represents a linkage formed between a functional group R^{X} on the linker and a “complementary” functional group R^{y} on the antibody, and n represents the number of drugs linked to, or drug-to-antibody ratio (DAR), of the ADC.

Specific embodiments of the various antibodies (Ab) that can comprise the ADCs include the various embodiments of MBMs described above.

In some specific embodiments of the ADCs and/or salts of structural formula (I), each D is the same and/or each L is the same.

Specific embodiments of cytotoxic and/or cytostatic agents (D) and linkers (L) that can comprise the ADCs of the disclosure, as well as the number of cytotoxic and/or cytostatic agents linked to the ADCs, are described in more detail below.

### 7.8.1. Cytotoxic and/or Cytostatic Agents

The cytotoxic and/or cytostatic agents may be any agents known to inhibit the growth and/or replication of and/or kill cells, and in particular cancer and/or tumor cells. Numerous agents having cytotoxic and/or cytostatic properties are known in the literature. Non-limiting examples of classes of cytotoxic and/or cytostatic agents include, by way of example and not limitation, radionuclides, alkylating agents, topoisomerase I inhibitors, topoisomerase II inhibitors, DNA intercalating agents (e.g., groove binding agents such as minor groove binders), RNA/DNA antimetabolites, cell cycle modulators, kinase inhibitors, protein synthesis inhibitors, histone deacetylase inhibitors, mitochondria inhibitors, and antimitotic agents.

Specific non-limiting examples of agents within certain of these various classes are provided below.

Alkylating Agents: asaley ((L-Leucine, N—[N-acetyl-4-[bis-(2-chloroethyl)amino]-DL-phenylalanyl]-, ethylester; NSC 167780; CAS Registry No. 3577897)); AZQ ((1,4-cyclohexadiene-1,4-dicarbamic acid, 2,5-bis(1-aziridinyl)-3,6-dioxo-, diethyl ester; NSC 182986; CAS Registry No. 57998682)); BCNU ((N,N′-Bis(2-chloroethyl)-N-nitrosourea; NSC 409962; CAS Registry No. 154938)); busulfan (1,4-butanediol dimethanesulfonate; NSC 750; CAS Registry No. 55981); (carboxyphthalato)platinum (NSC 27164; CAS Registry No. 65296813); CBDCA ((cis-(1,1-cyclobutanedicarboxylato)diammineplatinum(II)); NSC 241240; CAS Registry No. 41575944)); CCNU ((N-(2-chloroethyl)-N′-cyclohexyl-N-nitrosourea; NSC 79037; CAS Registry No. 13010474)); CHIP (iproplatin; NSC 256927); chlorambucil (NSC 3088; CAS Registry No. 305033); chlorozotocin ((2-[[[(2-chloroethyl) nitrosoamino]carbonyl]amino]-2-deoxy-D-glucopyranose; NSC 178248; CAS Registry No. 54749905)); cis-platinum (cisplatin; NSC 119875; CAS Registry No. 15663271); clomesone (NSC 338947; CAS Registry No. 88343720); cyanomorpholinodoxorubicin (NCS 357704; CAS Registry No. 88254073); cyclodisone (NSC 348948; CAS Registry No. 99591738); dianhydrogalactitol (5,6-diepoxydulcitol; NSC 132313; CAS Registry No. 23261203); fluorodopan ((5-[(2-chloroethyl)-(2-fluoroethyl)amino]-6-methyl-uracil; NSC 73754; CAS Registry No. 834913); hepsulfam (NSC 329680; CAS Registry No. 96892578); hycanthone (NSC 142982; CAS Registry No. 23255938); melphalan (NSC 8806; CAS Registry No. 3223072); methyl CCNU ((1-(2-chloroethyl)-3-(trans-4-methylcyclohexane)-1-nitrosourea; NSC 95441; 13909096); mitomycin C (NSC 26980; CAS Registry No. 50077); mitozolamide (NSC 353451; CAS Registry No. 85622953); nitrogen mustard ((bis(2-chloroethyl)methylamine hydrochloride; NSC 762; CAS Registry No. 55867); PCNU ((1-(2-chloroethyl)-3-(2,6-dioxo-3-piperidyl)-1-nitrosourea; NSC 95466; CAS Registry No. 13909029)); piperazine alkylator ((1-(2-chloroethyl)-4-(3-chloropropyl)-piperazine dihydrochloride; NSC 344007)); piperazinedione (NSC 135758; CAS Registry No. 41109802); pipobroman ((N,N-bis(3-bromopropionyl) piperazine; NSC 25154; CAS Registry No. 54911)); porfiromycin (N-methylmitomycin C; NSC 56410; CAS Registry No. 801525); spirohydantoin mustard (NSC 172112; CAS Registry No. 56605164); teroxirone (triglycidylisocyanurate; NSC 296934; CAS Registry No. 2451629); tetraplatin (NSC 363812; CAS Registry No. 62816982); thio-tepa (N,N′,N″-tri-1,2-ethanediylthio phosphoramide; NSC 6396; CAS Registry No. 52244); triethylenemelamine (NSC 9706; CAS Registry No. 51183); uracil nitrogen mustard (desmethyldopan; NSC 34462; CAS Registry No. 66751); Yoshi-864 ((bis(3-mesyloxy propyl)amine hydrochloride; NSC 102627; CAS Registry No. 3458228).

Topoisomerase I Inhibitors: camptothecin (NSC 94600; CAS Registry No. 7689-03-4); various camptothecin derivatives and analogs (for example, NSC 100880, NSC 603071, NSC 107124, NSC 643833, NSC 629971, NSC 295500, NSC 249910, NSC 606985, NSC 74028, NSC 176323, NSC295501, NSC606172, NSC606173, NSC 610458, NSC618939, NSC 610457, NSC 610459, NSC 606499, NSC 610456, NSC 364830, and NSC 606497); morpholinisoxorubicin (NSC 354646; CAS Registry No. 89196043); SN-38 (NSC 673596; CAS Registry No. 86639-52-3).

Topoisomerase II Inhibitors: doxorubicin (NSC 123127; CAS Registry No. 25316409); amonafide (benzisoquinolinedione; NSC 308847; CAS Registry No. 69408817); m-AMSA ((4′-(9-acridinylamino)-3′-methoxymethanesulfonanilide; NSC 249992; CAS Registry No. 51264143)); anthrapyrazole derivative ((NSC 355644); etoposide (VP-16; NSC 141540; CAS Registry No. 33419420); pyrazoloacridine ((pyrazolo[3,4,5-kl]acridine-2(6H)-propanamine, 9-methoxy-N, N-dimethyl-5-nitro-, monomethanesulfonate; NSC 366140; CAS Registry No. 99009219); bisantrene hydrochloride (NSC 337766; CAS Registry No. 71439684); daunorubicin (NSC 821151; CAS Registry No. 23541506); deoxydoxorubicin (NSC 267469; CAS Registry No. 63950061); mitoxantrone (NSC 301739; CAS Registry No. 70476823); menogaril (NSC 269148; CAS Registry No. 71628961); N,N-dibenzyl daunomycin (NSC 268242; CAS Registry No. 70878512); oxanthrazole (NSC 349174; CAS Registry No. 105118125); rubidazone (NSC 164011; CAS Registry No. 36508711); teniposide (VM-26; NSC 122819; CAS Registry No. 29767202).

DNA Intercalating Agents: anthramycin (CAS Registry No. 4803274); chicamycin A (CAS Registry No. 89675376); tomaymycin (CAS Registry No. 35050556); DC-81 (CAS Registry No. 81307246); sibiromycin (CAS Registry No. 12684332); pyrrolobenzodiazepine derivative (CAS Registry No. 945490095); SGD-1882 ((S)-2-(4-aminophenyl)-7-methoxy-8-(3-4(S)-7-methoxy-2-(4-methoxyphenyl)-5-oxo-5,11a-dihydro-1H-benzo[e]pyrrolo[1,2-a][1,4]diazepin-8-yl)oxy)propox-y)-1H-benzo[e]pyrrolo[1,2-a][1,4]diazepin-5(11aH)-one); SG2000 (SJG-136; (11aS,11a'S)-8,8′-(propane-1,3-diylbis(oxy))bis(7-methoxy-2-methylene-2,3-dihydro-1H-benzo[e]pyrrolo[1,2-a][1,4]diazepin-5(11aH)-one); NSC 694501; CAS Registry No. 232931576).

RNA/DNA Antimetabolites: L-alanosine (NSC 153353; CAS Registry No. 59163416); 5-azacytidine (NSC 102816; CAS Registry No. 320672); 5-fluorouracil (NSC 19893; CAS Registry No. 51218); acivicin (NSC 163501; CAS Registry No. 42228922); aminopterin derivative N-[2-chloro-5-[[(2,4-diamino-5-methyl-6-quinazolinyl)methyl]amino]benzoyl-]L-aspartic acid (NSC 132483); aminopterin derivative N-[4-[[(2,4-diamino-5-ethyl-6-quinazolinyl)methyl]amino]benzoyl]L-asparti-c acid (NSC 184692); aminopterin derivative N-[2-chloro-4-[[(2,4-diamino-6-pteridinyl)methyl]amino]benzoyl]L-aspartic acid monohydrate (NSC 134033); an antifo ((N^{α}-(4-amino-4-deoxypteroyl)-N⁷-hemiphthaloyl-L-ornithin-e; NSC 623017)); Baker's soluble antifol (NSC 139105; CAS Registry No. 41191042); dichlorallyl lawsone ((2-(3,3-dichloroallyl)-3-hydroxy-1,4-naphthoquinone; NSC 126771; CAS Registry No. 36417160); brequinar (NSC 368390; CAS Registry No. 96201886); ftorafur ((pro-drug; 5-fluoro-1-(tetrahydro-2-furyl)-uracil; NSC 148958; CAS Registry No. 37076689); 5,6-dihydro-5-azacytidine (NSC 264880; CAS Registry No. 62402317); methotrexate (NSC 740; CAS Registry No. 59052); methotrexate derivative (N-[[4-[[(2,4-diamino-6-pteridinyl)methyl]methylamino]-1-naphthalenyl]car-bonyl]L-glutamic acid; NSC 174121); PALA ((N-(phosphonoacetyl)-L-aspartate; NSC 224131; CAS Registry No. 603425565); pyrazofurin (NSC 143095; CAS Registry No. 30868305); trimetrexate (NSC 352122; CAS Registry No. 82952645).

DNA Antimetabolites: 3-HP (NSC 95678; CAS Registry No. 3814797); 2′-deoxy-5-fluorouridine (NSC 27640; CAS Registry No. 50919); 5-HP (NSC 107392; CAS Registry No. 19494894); α-TGDR (α-2′-deoxy-6-thioguanosine; NSC 71851 CAS Registry No. 2133815); aphidicolin glycinate (NSC 303812; CAS Registry No. 92802822); ara C (cytosine arabinoside; NSC 63878; CAS Registry No. 69749); 5-aza-2′-deoxycytidine (NSC 127716; CAS Registry No. 2353335); β-TGDR (β-2′-deoxy-6-thioguanosine; NSC 71261; CAS Registry No. 789617); cyclocytidine (NSC 145668; CAS Registry No. 10212256); guanazole (NSC 1895; CAS Registry No. 1455772); hydroxyurea (NSC 32065; CAS Registry No. 127071); inosine glycodialdehyde (NSC 118994; CAS Registry No. 23590990); macbecin II (NSC 330500; CAS Registry No. 73341738); pyrazoloimidazole (NSC 51143; CAS Registry No. 6714290); thioguanine (NSC 752; CAS Registry No. 154427); thiopurine (NSC 755; CAS Registry No. 50442).

Cell Cycle Modulators: silibinin (CAS Registry No. 22888-70-6); epigallocatechin gallate (EGCG; CAS Registry No. 989515); procyanidin derivatives (e.g., procyanidin A1 [CAS Registry No. 103883030], procyanidin B1 [CAS Registry No. 20315257], procyanidin B4 [CAS Registry No. 29106512], arecatannin B1 [CAS Registry No. 79763283]); isoflavones (e.g., genistein [4′,5,7-trihydroxyisoflavone; CAS Registry No. 446720], daidzein [4′,7-dihydroxyisoflavone, CAS Registry No. 486668]; indole-3-carbinol (CAS Registry No. 700061); quercetin (NSC 9219; CAS Registry No. 117395); estramustine (NSC 89201; CAS Registry No. 2998574); nocodazole (CAS Registry No. 31430189); podophyllotoxin (CAS Registry No. 518285); vinorelbine tartrate (NSC 608210; CAS Registry No. 125317397); cryptophycin (NSC 667642; CAS Registry No. 124689652).

Kinase Inhibitors: afatinib (CAS Registry No. 850140726); axitinib (CAS Registry No. 319460850); ARRY-438162 (binimetinib) (CAS Registry No. 606143899); bosutinib (CAS Registry No. 380843754); cabozantinib (CAS Registry No. 1140909483); ceritinib (CAS Registry No. 1032900256); crizotinib (CAS Registry No. 877399525); dabrafenib (CAS Registry No. 1195765457); dasatinib (NSC 732517; CAS Registry No. 302962498); erlotinib (NSC 718781; CAS Registry No. 183319699); everolimus (NSC 733504; CAS Registry No. 159351696); fostamatinib (NSC 745942; CAS Registry No. 901119355); gefitinib (NSC 715055; CAS Registry No. 184475352); ibrutinib (CAS Registry No. 936563961); imatinib (NSC 716051; CAS Registry No. 220127571); lapatinib (CAS Registry No. 388082788); lenvatinib (CAS Registry No. 857890392); mubritinib (CAS 366017096); nilotinib (CAS Registry No. 923288953); nintedanib (CAS Registry No. 656247175); palbociclib (CAS Registry No. 571190302); pazopanib (NSC 737754; CAS Registry No. 635702646); pegaptanib (CAS Registry No. 222716861); ponatinib (CAS Registry No. 1114544318); rapamycin (NSC 226080; CAS Registry No. 53123889); regorafenib (CAS Registry No. 755037037); AP 23573 (ridaforolimus) (CAS Registry No. 572924540); INCB018424 (ruxolitinib) (CAS Registry No. 1092939177); ARRY-142886 (selumetinib) (NSC 741078; CAS Registry No. 606143-52-6); sirolimus (NSC 226080; CAS Registry No. 53123889); sorafenib (NSC 724772; CAS Registry No. 475207591); sunitinib (NSC 736511; CAS Registry No. 341031547); tofacitinib (CAS Registry No. 477600752); temsirolimus (NSC 683864; CAS Registry No. 163635043); trametinib (CAS Registry No. 871700173); vandetanib (CAS Registry No. 443913733); vemurafenib (CAS Registry No. 918504651); SU6656 (CAS Registry No. 330161870); CEP-701 (lesaurtinib) (CAS Registry No. 111358884); XL019 (CAS Registry No. 945755566); PD-325901 (CAS Registry No. 391210109); PD-98059 (CAS Registry No. 167869218); ATP-competitive TORC1/TORC2 inhibitors including PI-103 (CAS Registry No. 371935749), PP242 (CAS Registry No. 1092351671), PP30 (CAS Registry No. 1092788094), Torin 1 (CAS Registry No. 1222998368), LY294002 (CAS Registry No. 154447366), XL-147 (CAS Registry No. 934526893), CAL-120 (CAS Registry No. 870281348), ETP-45658 (CAS Registry No. 1198357797), PX 866 (CAS Registry No. 502632668), GDC-0941 (CAS Registry No. 957054307), BGT226 (CAS Registry No. 1245537681), BEZ235 (CAS Registry No. 915019657), XL-765 (CAS Registry No. 934493762).

Protein Synthesis Inhibitors: acriflavine (CAS Registry No. 65589700); amikacin (NSC 177001; CAS Registry No. 39831555); arbekacin (CAS Registry No. 51025855); astromicin (CAS Registry No. 55779061); azithromycin (NSC 643732; CAS Registry No. 83905015); bekanamycin (CAS Registry No. 4696768); chlortetracycline (NSC 13252; CAS Registry No. 64722); clarithromycin (NSC 643733; CAS Registry No. 81103119); clindamycin (CAS Registry No. 18323449); clomocycline (CAS Registry No. 1181540); cycloheximide (CAS Registry No. 66819); dactinomycin (NSC 3053; CAS Registry No. 50760); dalfopristin (CAS Registry No. 112362502); demeclocycline (CAS Registry No. 127333); dibekacin (CAS Registry No. 34493986); dihydrostreptomycin (CAS Registry No. 128461); dirithromycin (CAS Registry No. 62013041); doxycycline (CAS Registry No. 17086281); emetine (NSC 33669; CAS Registry No. 483181); erythromycin (NSC 55929; CAS Registry No. 114078); flurithromycin (CAS Registry No. 83664208); framycetin (neomycin B; CAS Registry No. 119040); gentamycin (NSC 82261; CAS Registry No. 1403663); glycylcyclines, such as tigecycline (CAS Registry No. 220620097); hygromycin B (CAS Registry No. 31282049); isepamicin (CAS Registry No. 67814760); josamycin (NSC 122223; CAS Registry No. 16846245); kanamycin (CAS Registry No. 8063078); ketolides such as telithromycin (CAS Registry No. 191114484), cethromycin (CAS Registry No. 205110481), and solithromycin (CAS Registry No. 760981837); lincomycin (CAS Registry No. 154212); lymecycline (CAS Registry No. 992212); meclocycline (NSC 78502; CAS Registry No. 2013583); metacycline (rondomycin; NSC 356463; CAS Registry No. 914001); midecamycin (CAS Registry No. 35457808); minocycline (NSC 141993; CAS Registry No. 10118908); miocamycin (CAS Registry No. 55881077); neomycin (CAS Registry No. 119040); netilmicin (CAS Registry No. 56391561); oleandomycin (CAS Registry No. 3922905); oxazolidinones, such as eperezolid (CAS Registry No. 165800044), linezolid (CAS Registry No. 165800033), posizolid (CAS Registry No. 252260029), radezolid (CAS Registry No. 869884786), ranbezolid (CAS Registry No. 392659380), sutezolid (CAS Registry No. 168828588), tedizolid (CAS Registry No. 856867555); oxytetracycline (NSC 9169; CAS Registry No. 2058460); paromomycin (CAS Registry No. 7542372); penimepicycline (CAS Registry No. 4599604); peptidyl transferase inhibitors, e.g., chloramphenicol (NSC 3069; CAS Registry No. 56757) and derivatives such as azidamfenicol (CAS Registry No. 13838089), florfenicol (CAS Registry No. 73231342), and thiamphenicol (CAS Registry No. 15318453), and pleuromutilins such as retapamulin (CAS Registry No. 224452668), tiamulin (CAS Registry No. 55297955), valnemulin (CAS Registry No. 101312929); pirlimycin (CAS Registry No. 79548735); puromycin (NSC 3055; CAS Registry No. 53792); quinupristin (CAS Registry No. 120138503); ribostamycin (CAS Registry No. 53797356); rokitamycin (CAS Registry No. 74014510); rolitetracycline (CAS Registry No. 751973); roxithromycin (CAS Registry No. 80214831); sisomicin (CAS Registry No. 32385118); spectinomycin (CAS Registry No. 1695778); spiramycin (CAS Registry No. 8025818); streptogramins such as pristinamycin (CAS Registry No. 270076603), quinupristin/dalfopristin (CAS Registry No. 126602899), and virginiamycin (CAS Registry No. 11006761); streptomycin (CAS Registry No. 57921); tetracycline (NSC 108579; CAS Registry No. 60548); tobramycin (CAS Registry No. 32986564); troleandomycin (CAS Registry No. 2751099); tylosin (CAS Registry No. 1401690); verdamicin (CAS Registry No. 49863481).

Histone Deacetylase Inhibitors: abexinostat (CAS Registry No. 783355602); belinostat (NSC 726630; CAS Registry No. 414864009); chidamide (CAS Registry No. 743420022); entinostat (CAS Registry No. 209783802); givinostat (CAS Registry No. 732302997); mocetinostat (CAS Registry No. 726169739); panobinostat (CAS Registry No. 404950807); quisinostat (CAS Registry No. 875320299); resminostat (CAS Registry No. 864814880); romidepsin (CAS Registry No. 128517077); sulforaphane (CAS Registry No. 4478937); thioureidobutyronitrile (Kevetrin™; CAS Registry No. 6659890); valproic acid (NSC 93819; CAS Registry No. 99661); vorinostat (NSC 701852; CAS Registry No. 149647789); ACY-1215 (rocilinostat; CAS Registry No. 1316214524); CUDC-101 (CAS Registry No. 1012054599); CHR-2845 (tefinostat; CAS Registry No. 914382608); CHR-3996 (CAS Registry No. 1235859138); 4SC-202 (CAS Registry No. 910462430); CG200745 (CAS Registry No. 936221339); SB939 (pracinostat; CAS Registry No. 929016966).

Mitochondria Inhibitors: pancratistatin (NSC 349156; CAS Registry No. 96281311); rhodamine-123 (CAS Registry No. 63669709); edelfosine (NSC 324368; CAS Registry No. 70641519); d-alpha-tocopherol succinate (NSC 173849; CAS Registry No. 4345033); compound 11β (CAS Registry No. 865070377); aspirin (NSC 406186; CAS Registry No. 50782); ellipticine (CAS Registry No. 519233); berberine (CAS Registry No. 633658); cerulenin (CAS Registry No. 17397896); GX015-070 (Obatoclax®; 1H-Indole, 2-(2-((3,5-dimethyl-1H-pyrrol-2-yl)methylene)-3-methoxy-2H-pyrrol-5-yl)-; NSC 729280; CAS Registry No. 803712676); celastrol (tripterine; CAS Registry No. 34157830); metformin (NSC 91485; CAS Registry No. 1115704); Brilliant green (NSC 5011; CAS Registry No. 633034); ME-344 (CAS Registry No. 1374524556).

Antimitotic Agents: allocolchicine (NSC 406042); auristatins, such as MMAE (monomethyl auristatin E; CAS Registry No. 474645-27-7) and MMAF (monomethyl auristatin F; CAS Registry No. 745017-94-1; halichondrin B (NSC 609395); colchicine (NSC 757; CAS Registry No. 64868); cholchicine derivative (N-benzoyl-deacetyl benzamide; NSC 33410; CAS Registry No. 63989753); dolastatin 10 (NSC 376128; CAS Registry No 110417-88-4); maytansine (NSC 153858; CAS Registry No. 35846-53-8); rhozoxin (NSC 332598; CAS Registry No. 90996546); taxol (NSC 125973; CAS Registry No. 33069624); taxol derivative ((2′-N-[3-(dimethylamino)propyl]glutaramate taxol; NSC 608832); thiocolchicine (3-demethylthiocolchicine; NSC 361792); trityl cysteine (NSC 49842; CAS Registry No. 2799077); vinblastine sulfate (NSC 49842; CAS Registry No. 143679); vincristine sulfate (NSC 67574; CAS Registry No. 2068782).

Any of these agents that include or that may be modified to include a site of attachment to a MBM may be included in the ADCs disclosed herein.

In a specific embodiment, the cytotoxic and/or cytostatic agent is an antimitotic agent.

In another specific embodiment, the cytotoxic and/or cytostatic agent is an auristatin, for example, monomethyl auristatin E (“MMAE:) or monomethyl auristatin F (“MMAF”).

### 7.8.2. ADC Linkers

In the ADCs of the disclosure, the cytotoxic and/or cytostatic agents are linked to the MBM by way of ADC linkers. The ADC linker linking a cytotoxic and/or cytostatic agent to the MBM of an ADC may be short, long, hydrophobic, hydrophilic, flexible or rigid, or may be composed of segments that each independently have one or more of the above-mentioned properties such that the linker may include segments having different properties. The linkers may be polyvalent such that they covalently link more than one agent to a single site on the MBM, or monovalent such that covalently they link a single agent to a single site on the MBM.

As will be appreciated by skilled artisans, the ADC linkers link cytotoxic and/or cytostatic agents to the MBM by forming a covalent linkage to the cytotoxic and/or cytostatic agent at one location and a covalent linkage to the MBM at another. The covalent linkages are formed by reaction between functional groups on the ADC linker and functional groups on the agents and MBM. As used herein, the expression “ADC linker” is intended to include (i) unconjugated forms of the ADC linker that include a functional group capable of covalently linking the ADC linker to a cytotoxic and/or cytostatic agent and a functional group capable of covalently linking the ADC linker to a MBM; (ii) partially conjugated forms of the ADC linker that include a functional group capable of covalently linking the ADC linker to a MBM and that is covalently linked to a cytotoxic and/or cytostatic agent, or vice versa; and (iii) fully conjugated forms of the ADC linker that are covalently linked to both a cytotoxic and/or cytostatic agent and a MBM. In some specific embodiments of ADC linkers and ADCs of the disclosure, as well as synthons used to conjugate linker-agents to MBMs, moieties comprising the functional groups on the ADC linker and covalent linkages formed between the ADC linker and MBM are specifically illustrated as Rₓ and XY, respectively.

The ADC linkers are, but need not be, chemically stable to conditions outside the cell, and may be designed to cleave, immolate and/or otherwise specifically degrade inside the cell. Alternatively, ADC linkers that are not designed to specifically cleave or degrade inside the cell may be used. Choice of stable versus unstable ADC linker may depend upon the toxicity of the cytotoxic and/or cytostatic agent. For agents that are toxic to normal cells, stable linkers can be used. Agents that are selective or targeted and have lower toxicity to normal cells may utilize, chemical stability of the ADC linker to the extracellular milieu is less important. A wide variety of ADC linkers useful for linking drugs to MBMs in the context of ADCs are known in the art. Any of these ADC linkers, as well as other ADC linkers, may be used to link the cytotoxic and/or cytostatic agents to the MBM of the ADCs of the disclosure.

Exemplary polyvalent ADC linkers that may be used to link many cytotoxic and/or cytostatic agents to a single MBM molecule are described, for example, in WO 2009/073445; WO 2010/068795; WO 2010/138719; WO 2011/120053; WO 2011/171020; WO 2013/096901; WO 2014/008375; WO 2014/093379; WO 2014/093394; WO 2014/093640. For example, the Fleximer linker technology developed by Mersana et al. has the potential to enable high-DAR ADCs with good physicochemical properties. As shown below, the Mersana technology is based on incorporating drug molecules into a solubilizing poly-acetal backbone via a sequence of ester bonds. The methodology renders highly-loaded ADCs (DAR up to 20) while maintaining good physicochemical properties.

Additional examples of dendritic type linkers can be found in US 2006/116422; US 2005/271615; de Groot et al., 2003, Angew. Chem. Int. Ed. 42:4490-4494; Amir et al., 2003, Angew. Chem. Int. Ed. 42:4494-4499; Shamis et al., 2004, J. Am. Chem. Soc. 126:1726-1731; Sun et al., 2002, Bioorganic & Medicinal Chemistry Letters 12:2213-2215; Sun et al., 2003, Bioorganic & Medicinal Chemistry 11:1761-1768; King et al., 2002, Tetrahedron Letters 43:1987-1990.

Exemplary monovalent ADC linkers that may be used are described, for example, in Nolting, 2013, Antibody-Drug Conjugates, Methods in Molecular Biology 1045:71-100; Kitson et al., 2013, CROs-MOs-Chemica-ggi-Chemistry Today 31(4):30-38; Ducry et al., 2010, Bioconjugate Chem. 21:5-13; Zhao et al., 2011, J. Med. Chem. 54:3606-3623; U.S. Pat. Nos. 7,223,837; 8,568,728; 8,535,678; and WO2004010957.

By way of example and not limitation, some cleavable and noncleavable ADC linkers that may be included in the ADCs of the disclosure are described below.

### 7.8.2.1. Cleavable ADC Linkers

In certain embodiments, the ADC linker selected is cleavable in vivo. Cleavable ADC linkers may include chemically or enzymatically unstable or degradable linkages. Cleavable ADC linkers generally rely on processes inside the cell to liberate the drug, such as reduction in the cytoplasm, exposure to acidic conditions in the lysosome, or cleavage by specific proteases or other enzymes within the cell. Cleavable ADC linkers generally incorporate one or more chemical bonds that are either chemically or enzymatically cleavable while the remainder of the ADC linker is noncleavable. In certain embodiments, an ADC linker comprises a chemically labile group such as hydrazone and/or disulfide groups. Linkers comprising chemically labile groups exploit differential properties between the plasma and some cytoplasmic compartments. The intracellular conditions to facilitate drug release for hydrazone containing ADC linkers are the acidic environment of endosomes and lysosomes, while the disulfide containing ADC linkers are reduced in the cytosol, which contains high thiol concentrations, e.g., glutathione. In certain embodiments, the plasma stability of an ADC linker comprising a chemically labile group may be increased by introducing steric hindrance using substituents near the chemically labile group.

Acid-labile groups, such as hydrazone, remain intact during systemic circulation in the blood's neutral pH environment (pH 7.3-7.5) and undergo hydrolysis and release the drug once the ADC is internalized into mildly acidic endosomal (pH 5.0-6.5) and lysosomal (pH 4.5-5.0) compartments of the cell. This pH dependent release mechanism has been associated with nonspecific release of the drug. To increase the stability of the hydrazone group of the ADC linker, the ADC linker may be varied by chemical modification, e.g., substitution, allowing tuning to achieve more efficient release in the lysosome with a minimized loss in circulation.

Hydrazone-containing ADC linkers may contain additional cleavage sites, such as additional acid-labile cleavage sites and/or enzymatically labile cleavage sites. ADCs including exemplary hydrazone-containing ADC linkers include the following structures:

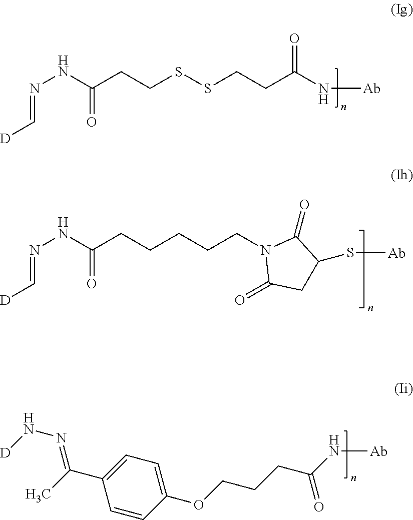

where D and Ab represent the cytotoxic and/or cytostatic agent (drug) and Ab, respectively, and n represents the number of drug-ADC linkers linked to the MBM. In certain ADC linkers such as linker (Ig), the ADC linker comprises two cleavable groups—a disulfide and a hydrazone moiety. For such ADC linkers, effective release of the unmodified free drug requires acidic pH or disulfide reduction and acidic pH. Linkers such as (Ih) and (Ii) have been shown to be effective with a single hydrazone cleavage site.

Additional ADC linkers which remain intact during systemic circulation and undergo hydrolysis and release the drug when the ADC is internalized into acidic cellular compartments include carbonates. Such ADC linkers can be useful in cases where the cytotoxic and/or cytostatic agent can be covalently attached through an oxygen.

Other acid-labile groups that may be included in ADC linkers include cis-aconityl-containing ADC linkers. cis-Aconityl chemistry uses a carboxylic acid juxtaposed to an amide bond to accelerate amide hydrolysis under acidic conditions.

Cleavable ADC linkers may also include a disulfide group. Disulfides are thermodynamically stable at physiological pH and are designed to release the drug upon internalization inside cells, where the cytosol provides a significantly more reducing environment compared to the extracellular environment. Scission of disulfide bonds generally requires the presence of a cytoplasmic thiol cofactor, such as (reduced) glutathione (GSH), such that disulfide-containing ADC linkers are reasonably stable in circulation, selectively releasing the drug in the cytosol. The intracellular enzyme protein disulfide isomerase, or similar enzymes capable of cleaving disulfide bonds, may also contribute to the preferential cleavage of disulfide bonds inside cells. GSH is reported to be present in cells in the concentration range of 0.5-10 mM compared with a significantly lower concentration of GSH or cysteine, the most abundant low-molecular weight thiol, in circulation at approximately 5 Tumor cells, where irregular blood flow leads to a hypoxic state, result in enhanced activity of reductive enzymes and therefore even higher glutathione concentrations. In certain embodiments, the in vivo stability of a disulfide-containing ADC linker may be enhanced by chemical modification of the ADC linker, e.g., use of steric hindrance adjacent to the disulfide bond.

ADCs including exemplary disulfide-containing ADC linkers include the following structures:

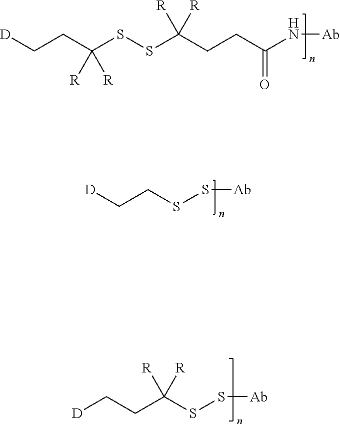

where D and Ab represent the drug and MBM, respectively, n represents the number of drug-ADC linkers linked to the MBM and R is independently selected at each occurrence from hydrogen or alkyl, for example. In certain embodiments, increasing steric hindrance adjacent to the disulfide bond increases the stability of the ADC linker. Structures such as (Ij) and (II) show increased in vivo stability when one or more R groups is selected from a lower alkyl such as methyl.

Another type of cleavable ADC linker that may be used is an ADC linker that is specifically cleaved by an enzyme. Such ADC linkers are typically peptide-based or include peptidic regions that act as substrates for enzymes. Peptide based ADC linkers tend to be more stable in plasma and extracellular milieu than chemically labile ADC linkers. Peptide bonds generally have good serum stability, as lysosomal proteolytic enzymes have very low activity in blood due to endogenous inhibitors and the unfavorably high pH value of blood compared to lysosomes. Release of a drug from a MBM occurs specifically due to the action of lysosomal proteases, e.g., cathepsin and plasmin. These proteases may be present at elevated levels in certain tumor cells.

In exemplary embodiments, the cleavable peptide is selected from tetrapeptides such as Gly-Phe-Leu-Gly, (SEQ ID NO: 725), Ala-Leu-Ala-Leu (SEQ ID NO: 726) or dipeptides such as Val-Cit, Val-Ala, Met-(D)Lys, Asn-(D)Lys, Val-(D)Asp, Phe-Lys, Ile-Val, Asp-Val, His-Val, NorVal-(D)Asp, Ala-(D)Asp 5, Met-Lys, Asn-Lys, lie-Pro, Me3Lys-Pro, PhenylGly-(D)Lys, Met-(D)Lys, Asn-(D)Lys, Pro-(D)Lys, Met-(D)Lys, Asn-(D)Lys, AM Met-(D)Lys, Asn-(D)Lys, AW Met-(D)Lys, and Asn-(D)Lys. In certain embodiments, dipeptides can be selected over longer polypeptides due to hydrophobicity of the longer peptides.

A variety of dipeptide-based cleavable ADC linkers useful for linking drugs such as doxorubicin, mitomycin, camptothecin, pyrrolobenzodiazepine, tallysomycin and auristatin/auristatin family members to MBMs have been described (see, Dubowchik et al., 1998, J. Org. Chem. 67:1866-1872; Dubowchik et al., 1998, Bioorg. Med. Chem. Lett. 8(21):3341-3346; Walker et al., 2002, Bioorg. Med. Chem. Lett. 12:217-219; Walker et al., 2004, Bioorg. Med. Chem. Lett. 14:4323-4327; Sutherland et al., 2013, Blood 122: 1455-1463; and Francisco et al., 2003, Blood 102:1458-1465). All of these dipeptide ADC linkers, or modified versions of these dipeptide ADC linkers, may be used in the ADCs of the disclosure. Other dipeptide ADC linkers that may be used include those found in ADCs such as Seattle Genetics' Brentuximab Vendotin SGN-35 (Adcetris™), Seattle Genetics SGN-75 (anti-CD-70, Val-Cit-monomethyl auristatin F(MMAF), Seattle Genetics SGN-CD33A (anti-CD-33, Val-Ala-(SGD-1882)), Celldex Therapeutics glembatumumab (CDX-011) (anti-NMB, Val-Cit-monomethyl auristatin E (MMAE), and Cytogen PSMA-ADC (PSMA-ADC-1301) (anti-PSMA, Val-Cit-MMAE).

Enzymatically cleavable ADC linkers may include a self-immolative spacer to spatially separate the drug from the site of enzymatic cleavage. The direct attachment of a drug to a peptide ADC linker can result in proteolytic release of an amino acid adduct of the drug, thereby impairing its activity. The use of a self-immolative spacer allows for the elimination of the fully active, chemically unmodified drug upon amide bond hydrolysis.

One self-immolative spacer is the bifunctional para-aminobenzyl alcohol group, which is linked to the peptide through the amino group, forming an amide bond, while amine containing drugs may be attached through carbamate functionalities to the benzylic hydroxyl group of the ADC linker (PABC). The resulting prodrugs are activated upon protease-mediated cleavage, leading to a 1,6-elimination reaction releasing the unmodified drug, carbon dioxide, and remnants of the ADC linker group. The following scheme depicts the fragmentation of p-amidobenzyl ether and release of the drug:

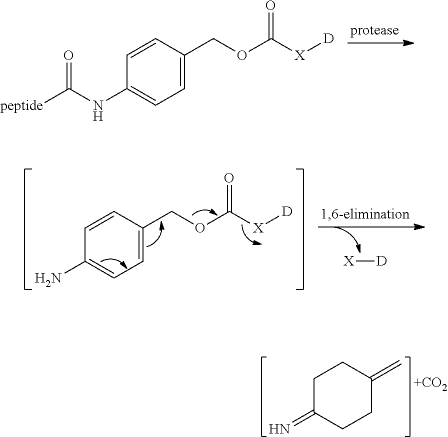

where X-D represents the unmodified drug.

Heterocyclic variants of this self-immolative group have also been described. See for example, U.S. Pat. No. 7,989,434.

In some embodiments, the enzymatically cleavable ADC linker is a β-glucuronic acid-based ADC linker. Facile release of the drug may be realized through cleavage of the β-glucuronide glycosidic bond by the lysosomal enzyme β-glucuronidase. This enzyme is present abundantly within lysosomes and is overexpressed in some tumor types, while the enzyme activity outside cells is low. β-Glucuronic acid-based ADC linkers may be used to circumvent the tendency of an ADC to undergo aggregation due to the hydrophilic nature of β-glucuronides. In some embodiments, β-glucuronic acid-based ADC linkers can be used as ADC linkers for ADCs linked to hydrophobic drugs. The following scheme depicts the release of the drug from and ADC containing a β-glucuronic acid-based ADC linker:

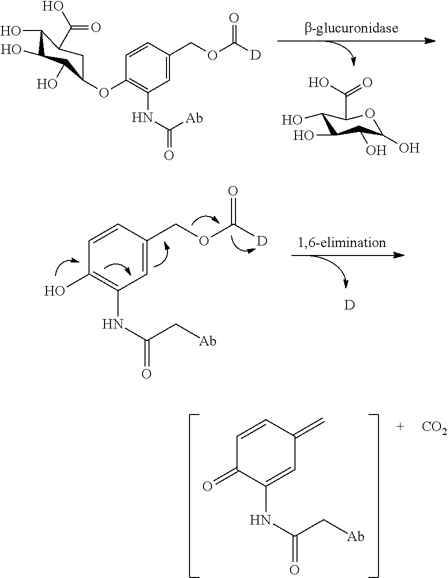

A variety of cleavable β-glucuronic acid-based ADC linkers useful for linking drugs such as auristatins, camptothecin and doxorubicin analogues, CBI minor-groove binders, and psymberin to MBMs have been described (see, see Nolting, Chapter 5 “Linker Technology in Antibody-Drug Conjugates,” In: Antibody-Drug Conjugates: Methods in Molecular Biology, vol. 1045, pp. 71-100, Laurent Ducry (Ed.), Springer Science & Business Medica, LLC, 2013; Jeffrey et al., 2006, Bioconjug. Chem. 17:831-840; Jeffrey et al., 2007, Bioorg. Med. Chem. Lett. 17:2278-2280; and Jiang et al., 2005, J. Am. Chem. Soc. 127:11254-11255). All of these β-glucuronic acid-based ADC linkers may be used in the ADCs of the disclosure.

Additionally, cytotoxic and/or cytostatic agents containing a phenol group can be covalently bonded to an ADC linker through the phenolic oxygen. One such ADC linker, described in WO 2007/089149, relies on a methodology in which a diamino-ethane “SpaceLink” is used in conjunction with traditional “PABO”-based self-immolative groups to deliver phenols. The cleavage of the ADC linker is depicted schematically below, where D represents a cytotoxic and/or cytostatic agent having a phenolic hydroxyl group.

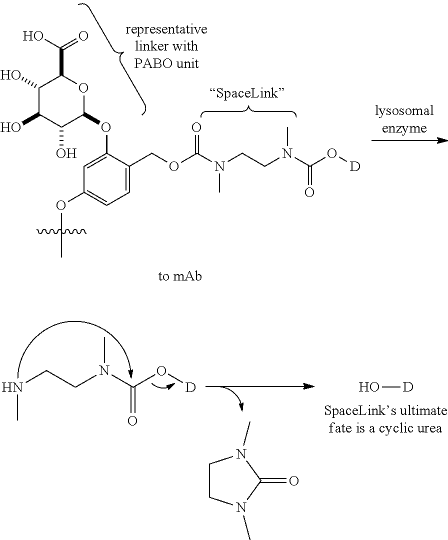

Cleavable ADC linkers may include noncleavable portions or segments, and/or cleavable segments or portions may be included in an otherwise non-cleavable ADC linker to render it cleavable. By way of example only, polyethylene glycol (PEG) and related polymers may include cleavable groups in the polymer backbone. For example, a polyethylene glycol or polymer ADC linker may include one or more cleavable groups such as a disulfide, a hydrazone or a dipeptide.

Other degradable linkages that may be included in ADC linkers include ester linkages formed by the reaction of PEG carboxylic acids or activated PEG carboxylic acids with alcohol groups on a biologically active agent, where such ester groups generally hydrolyze under physiological conditions to release the biologically active agent. Hydrolytically degradable linkages include, but are not limited to, carbonate linkages; imine linkages resulting from reaction of an amine and an aldehyde; phosphate ester linkages formed by reacting an alcohol with a phosphate group; acetal linkages that are the reaction product of an aldehyde and an alcohol; orthoester linkages that are the reaction product of a formate and an alcohol; and oligonucleotide linkages formed by a phosphoramidite group, including but not limited to, at the end of a polymer, and a 5′ hydroxyl group of an oligonucleotide.

In certain embodiments, the ADC linker comprises an enzymatically cleavable peptide moiety, for example, an ADC linker comprising structural formula (IVa) or (IVb):

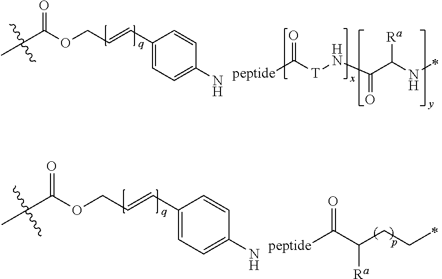

or a salt thereof, where: peptide represents a peptide (illustrated C→N and not showing the carboxy and amino “termini”) cleavable by a lysosomal enzyme; T represents a polymer comprising one or more ethylene glycol units or an alkylene chain, or combinations thereof; R^{a} is selected from hydrogen, alkyl, sulfonate and methyl sulfonate; p is an integer ranging from 0 to 5; q is 0 or 1; x is 0 or 1; y is 0 or 1; / represents the point of attachment of the ADC linker to a cytotoxic and/or cytostatic agent; and * represents the point of attachment to the remainder of the ADC linker.

In certain embodiments, the peptide is selected from a tripeptide or a dipeptide. In particular embodiments, the dipeptide is selected from: Val-Cit; Cit-Val; Ala-Ala; Ala-Cit; Cit-Ala; Asn-Cit; Cit-Asn; Cit-Cit; Val-Glu; Glu-Val; Ser-Cit; Cit-Ser; Lys-Cit; Cit-Lys; Asp-Cit; Cit-Asp; Ala-Val; Val-Ala; Phe-Lys; Val-Lys; Ala-Lys; Phe-Cit; Leu-Cit; Ile-Cit; Phe-Arg; and Trp-Cit. In certain embodiments, the dipeptide is selected from: Cit-Val; and Ala-Val.

Specific exemplary embodiments of ADC linkers according to structural formula (IVa) that may be included in the ADCs of the disclosure include the ADC linkers illustrated below (as illustrated, the ADC linkers include a group suitable for covalently linking the ADC linker to a MBM):

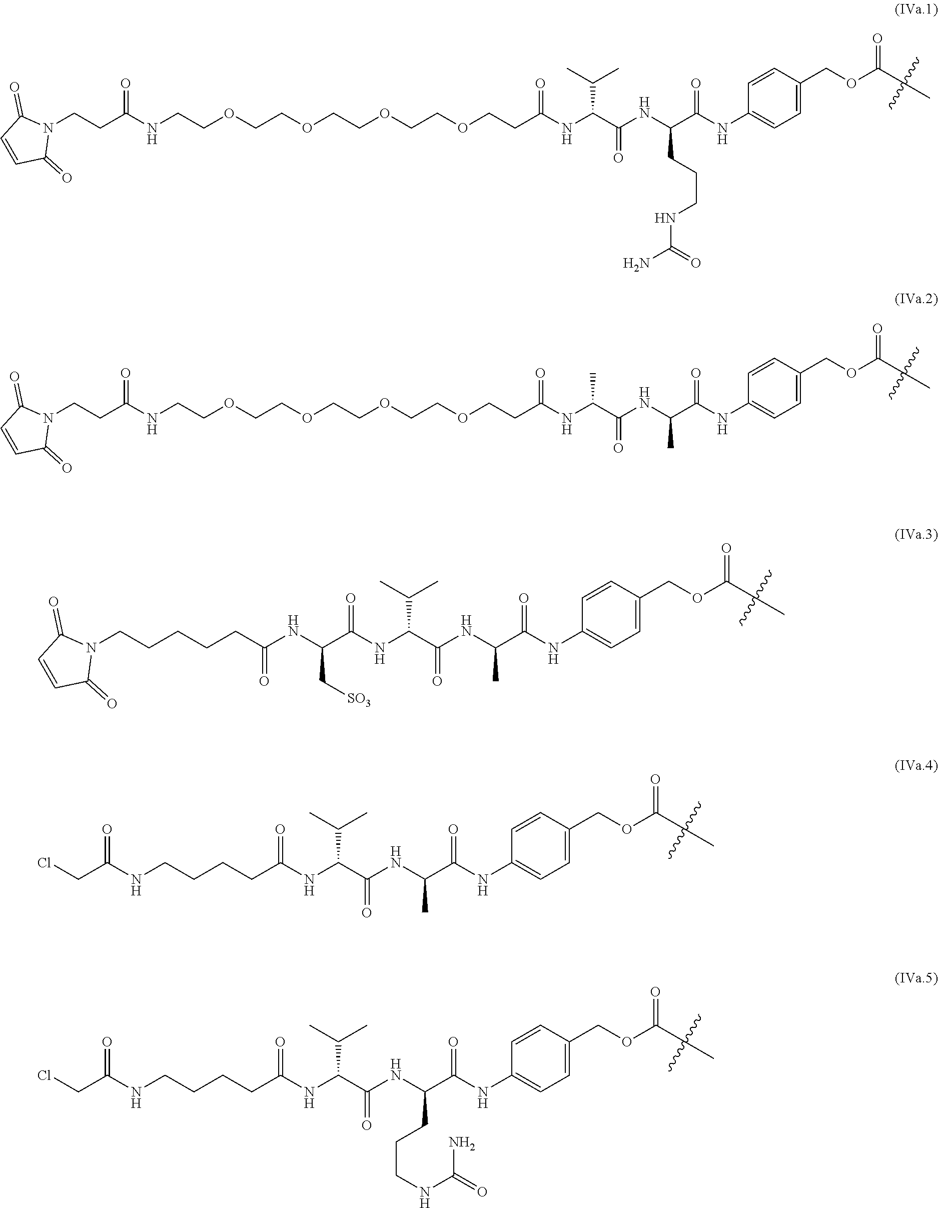

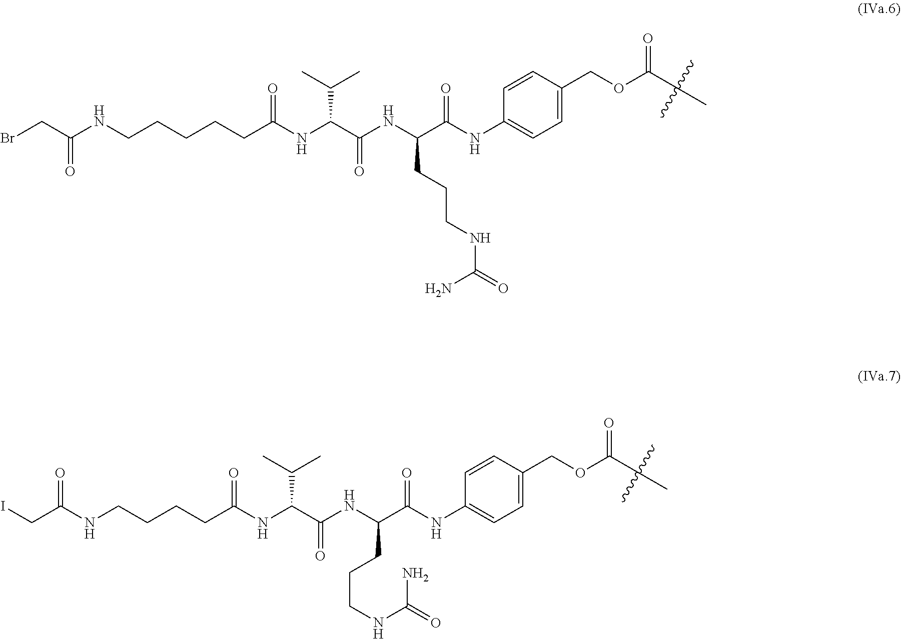 [SMILES: *C(=O)OCc1ccc(NC(=O)[C@@H](CCCNC(N)=O)NC(=O)[C@H](NC(=O)CCCCCNC(=O)CBr)C(C)C)cc1 ; *C(=O)OCc1ccc(NC(=O)[C@@H](CCCNC(N)=O)NC(=O)[C@H](NC(=O)CCCCNC(=O)CI)C(C)C)cc1]

Specific exemplary embodiments of ADC linkers according to structural formula (IVb) that may be included in the ADCs of the disclosure include the ADC linkers illustrated below (as illustrated, the ADC linkers include a group suitable for covalently linking the ADC linker to a MBM):

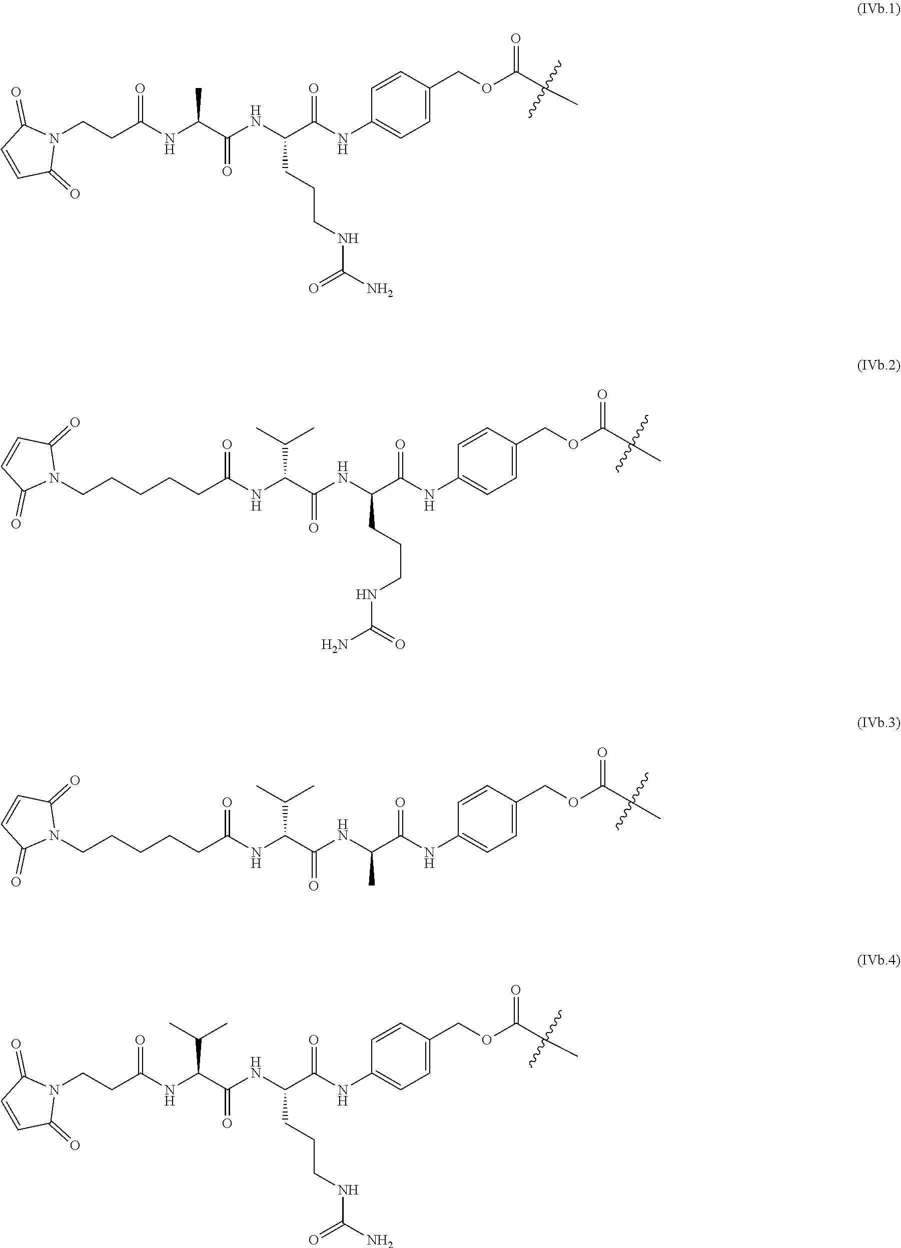 [SMILES: *C(=O)OCc1ccc(NC(=O)[C@H](CCCNC(N)=O)NC(=O)[C@H](C)NC(=O)CCN2C(=O)C=CC2=O)cc1 ; *C(=O)OCc1ccc(NC(=O)[C@@H](CCCNC(N)=O)NC(=O)[C@H](NC(=O)CCCCCN2C(=O)C=CC2=O)C(C)C)cc1 ; *C(=O)OCc1ccc(NC(=O)[C@@H](C)NC(=O)[C@H](NC(=O)CCCCCN2C(=O)C=CC2=O)C(C)C)cc1 ; *C(=O)OCc1ccc(NC(=O)[C@H](CCCNC(N)=O)NC(=O)[C@@H](NC(=O)CCN2C(=O)C=CC2=O)C(C)C)cc1]

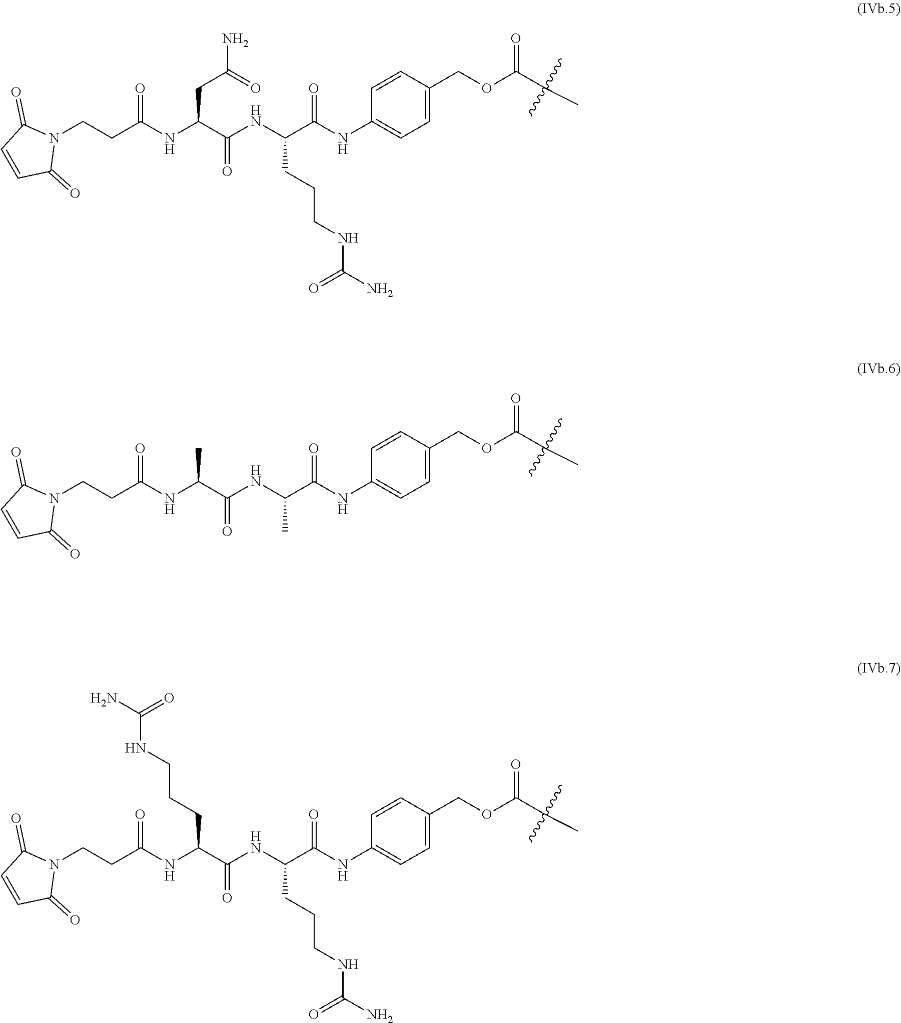 [SMILES: *C(=O)OCc1ccc(NC(=O)[C@H](CCCNC(N)=O)NC(=O)[C@H](CC(N)=O)NC(=O)CCN2C(=O)C=CC2=O)cc1 ; *C(=O)OCc1ccc(NC(=O)[C@H](C)NC(=O)[C@H](C)NC(=O)CCN2C(=O)C=CC2=O)cc1 ; *C(=O)OCc1ccc(NC(=O)[C@H](CCCNC(N)=O)NC(=O)[C@H](CCCNC(N)=O)NC(=O)CCN2C(=O)C=CC2=O)cc1]

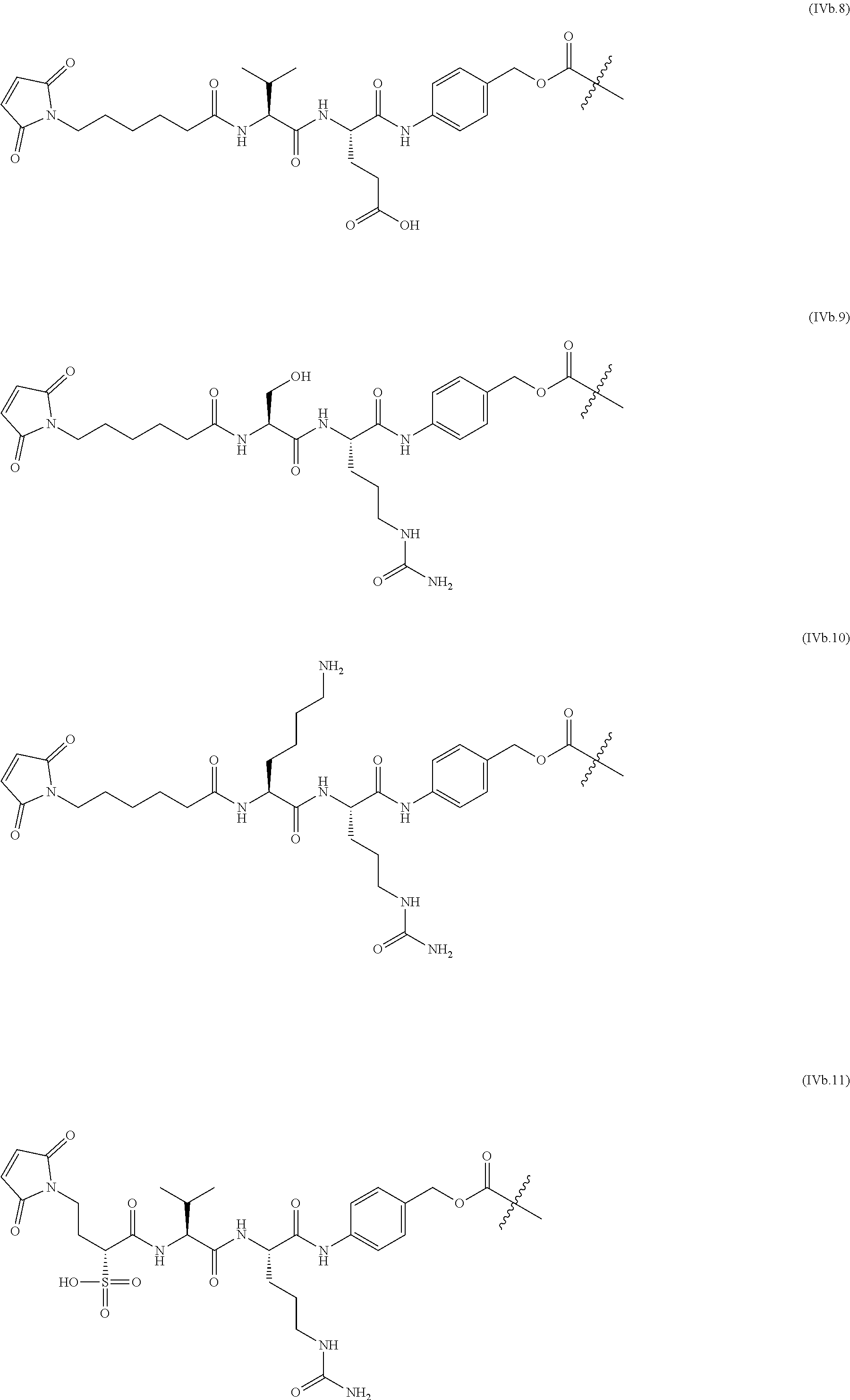 [SMILES: *C(=O)OCc1ccc(NC(=O)[C@H](CCC(=O)O)NC(=O)[C@@H](NC(=O)CCCCCN2C(=O)C=CC2=O)C(C)C)cc1 ; *C(=O)OCc1ccc(NC(=O)[C@H](CCCNC(N)=O)NC(=O)[C@H](CO)NC(=O)CCCCCN2C(=O)C=CC2=O)cc1 ; *C(=O)OCc1ccc(NC(=O)[C@H](CCCNC(N)=O)NC(=O)[C@H](CCCCN)NC(=O)CCCCCN2C(=O)C=CC2=O)cc1 ; *C(=O)OCc1ccc(NC(=O)[C@H](CCCNC(N)=O)NC(=O)[C@@H](NC(=O)[C@@H](CCN2C(=O)C=CC2=O)S(=O)(=O)O)C(C)C)cc1]

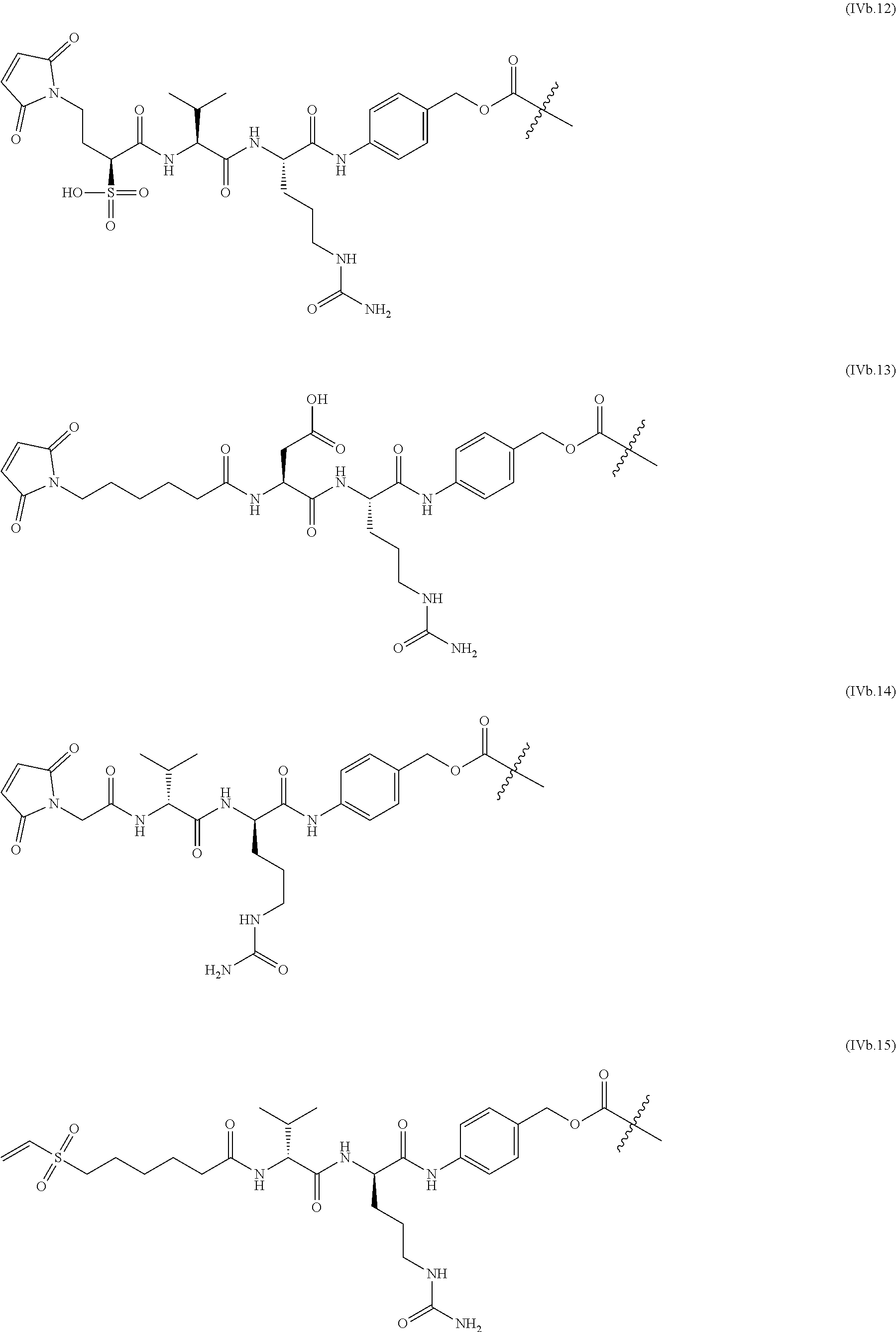 [SMILES: *C(=O)OCc1ccc(NC(=O)[C@H](CCCNC(N)=O)NC(=O)[C@@H](NC(=O)[C@H](CCN2C(=O)C=CC2=O)S(=O)(=O)O)C(C)C)cc1 ; *C(=O)OCc1ccc(NC(=O)[C@H](CCCNC(N)=O)NC(=O)[C@H](CC(=O)O)NC(=O)CCCCCN2C(=O)C=CC2=O)cc1 ; *C(=O)OCc1ccc(NC(=O)[C@@H](CCCNC(N)=O)NC(=O)[C@H](NC(=O)CN2C(=O)C=CC2=O)C(C)C)cc1 ; *C(=O)OCc1ccc(NC(=O)[C@@H](CCCNC(N)=O)NC(=O)[C@H](NC(=O)CCCCCS(=O)(=O)C=C)C(C)C)cc1]

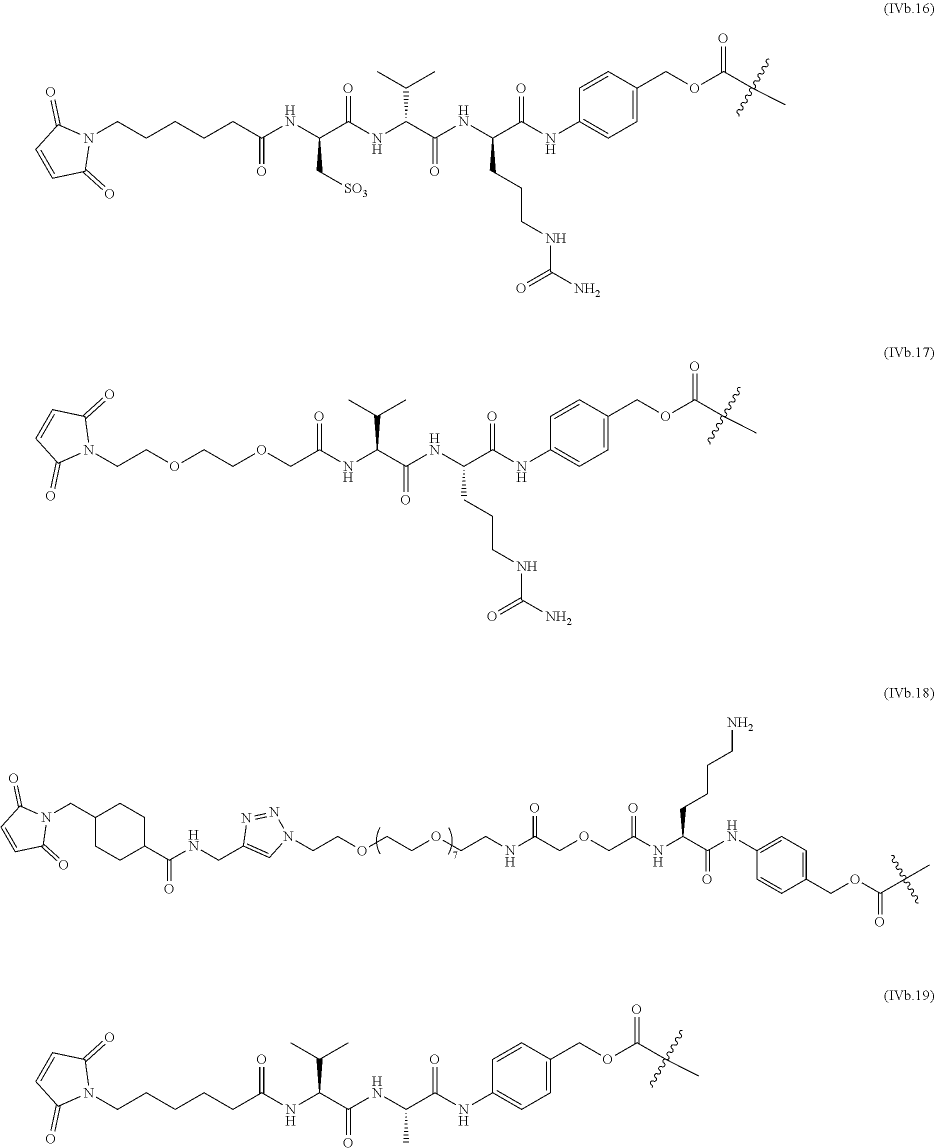

In certain embodiments, the ADC linker comprises an enzymatically cleavable peptide moiety, for example, an ADC linker comprising structural formula (IVc) or (IVd):

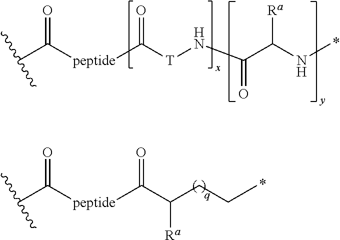

or a salt thereof, where: peptide represents a peptide (illustrated C→N and not showing the carboxy and amino “termini”) cleavable by a lysosomal enzyme; T represents a polymer comprising one or more ethylene glycol units or an alkylene chain, or combinations thereof; R^{a} is selected from hydrogen, alkyl, sulfonate and methyl sulfonate; p is an integer ranging from 0 to 5; q is 0 or 1; x is 0 or 1; y is 0 or 1; ⋅x [symbol] represents the point of attachment of the ADC linker to a cytotoxic and/or cytostatic agent; and * represents the point of attachment to the remainder of the ADC linker.

Specific exemplary embodiments of ADC linkers according to structural formula (IVc) that may be included in the ADCs of the disclosure include the ADC linkers illustrated below (as illustrated, the ADC linkers include a group suitable for covalently linking the ADC linker to a MBM):

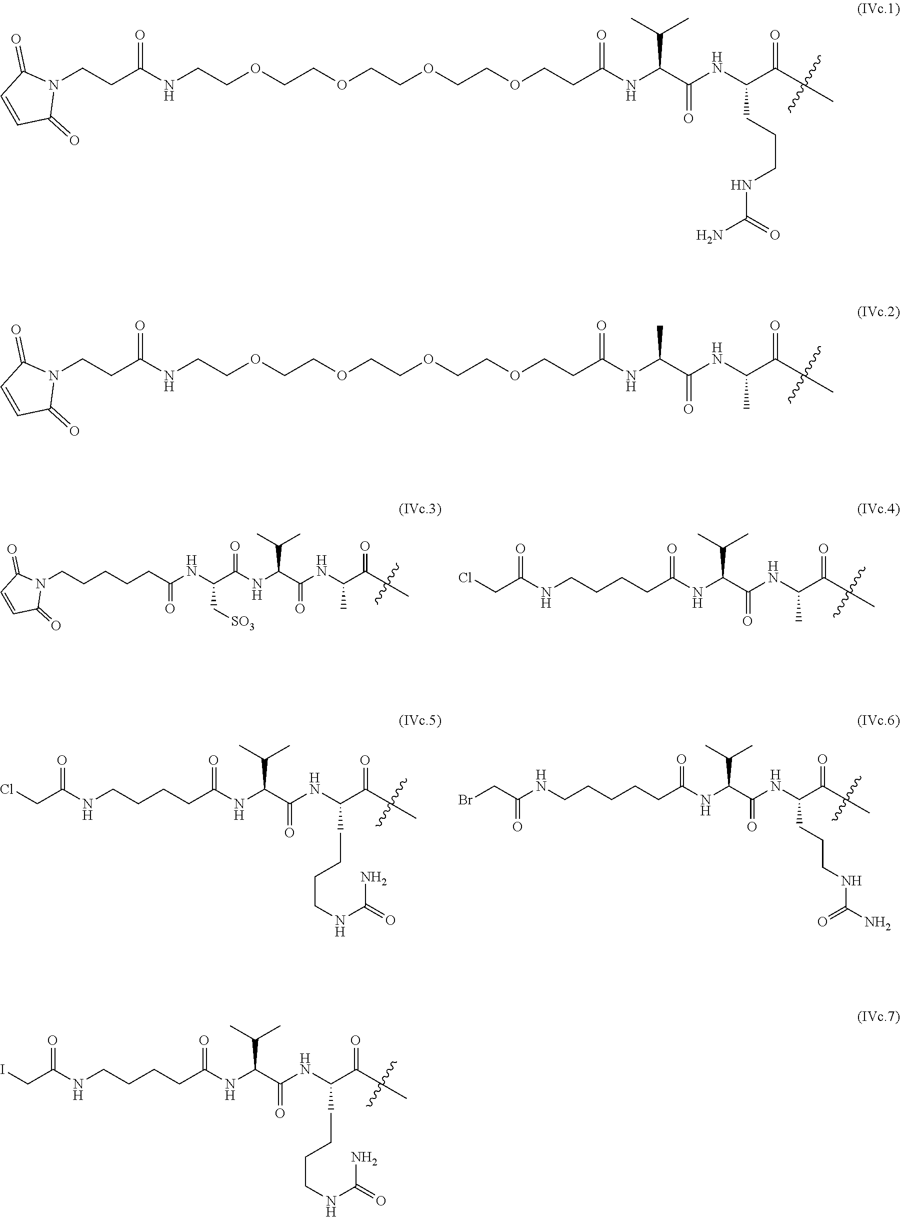

Specific exemplary embodiments of ADC linkers according to structural formula (IVd) that may be included in the ADCs of the disclosure include the ADC linkers illustrated below (as illustrated, the ADC linkers include a group suitable for covalently linking the ADC linker to a MBM):

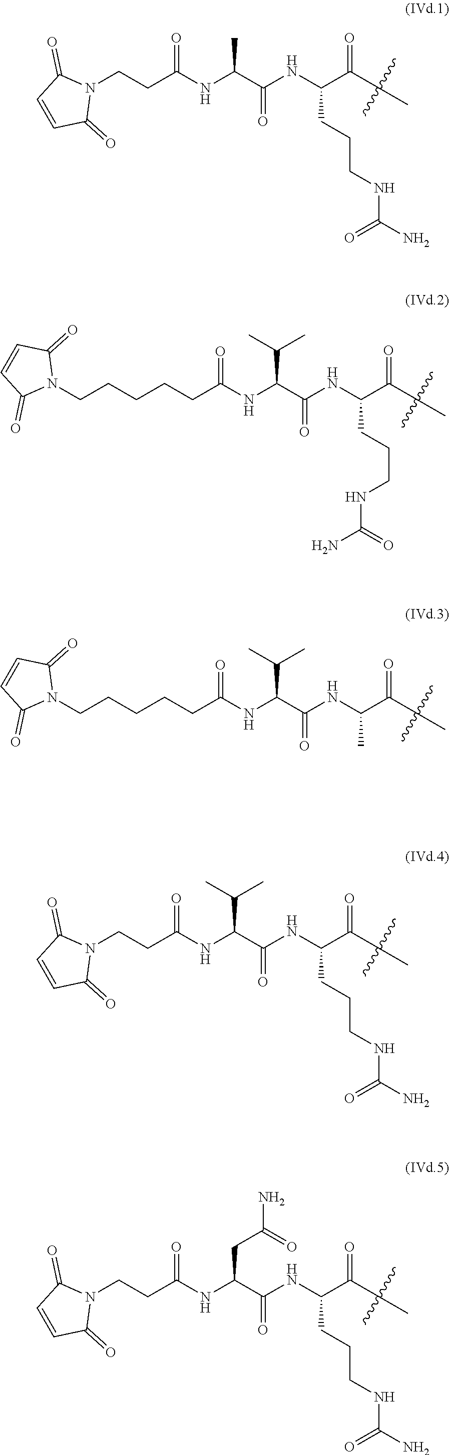 [SMILES: *C(=O)[C@H](CCCNC(N)=O)NC(=O)[C@H](C)NC(=O)CCN1C(=O)C=CC1=O ; *C(=O)[C@H](CCCNC(N)=O)NC(=O)[C@@H](NC(=O)CCCCCN1C(=O)C=CC1=O)C(C)C ; *C(=O)[C@H](C)NC(=O)[C@@H](NC(=O)CCCCCN1C(=O)C=CC1=O)C(C)C ; *C(=O)[C@H](CCCNC(N)=O)NC(=O)[C@@H](NC(=O)CCN1C(=O)C=CC1=O)C(C)C ; *C(=O)[C@H](CCCNC(N)=O)NC(=O)[C@H](CC(N)=O)NC(=O)CCN1C(=O)C=CC1=O]

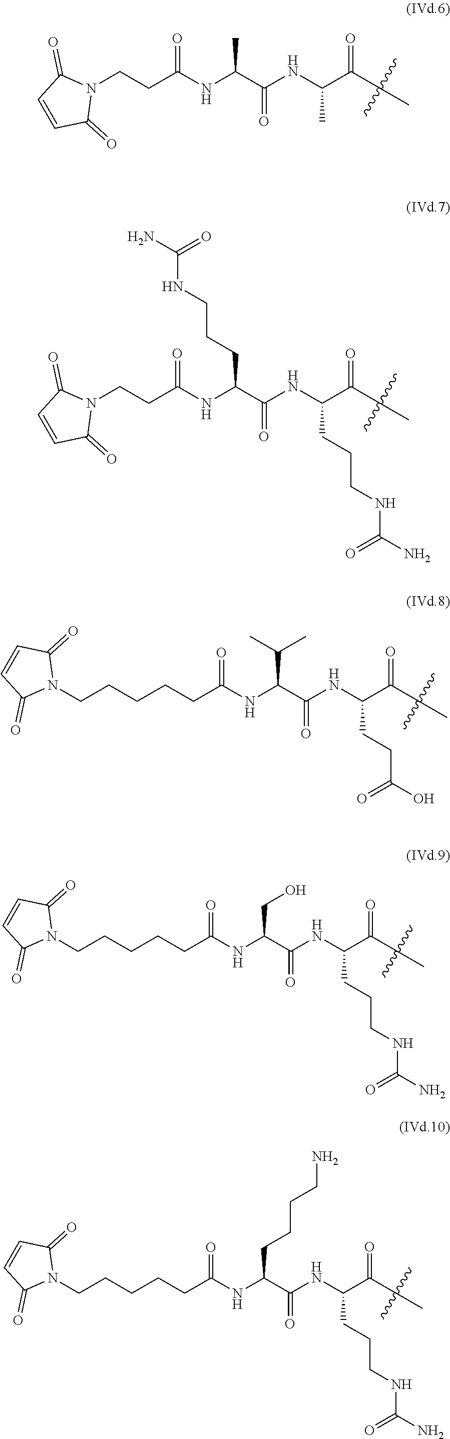 [SMILES: *C(=O)[C@H](C)NC(=O)[C@H](C)NC(=O)CCN1C(=O)C=CC1=O ; *C(=O)[C@H](CCCNC(N)=O)NC(=O)[C@H](CCCNC(N)=O)NC(=O)CCN1C(=O)C=CC1=O ; *C(=O)[C@H](CCC(=O)O)NC(=O)[C@@H](NC(=O)CCCCCN1C(=O)C=CC1=O)C(C)C ; *C(=O)[C@H](CCCNC(N)=O)NC(=O)[C@H](CO)NC(=O)CCCCCN1C(=O)C=CC1=O ; *C(=O)[C@H](CCCNC(N)=O)NC(=O)[C@H](CCCCN)NC(=O)CCCCCN1C(=O)C=CC1=O]

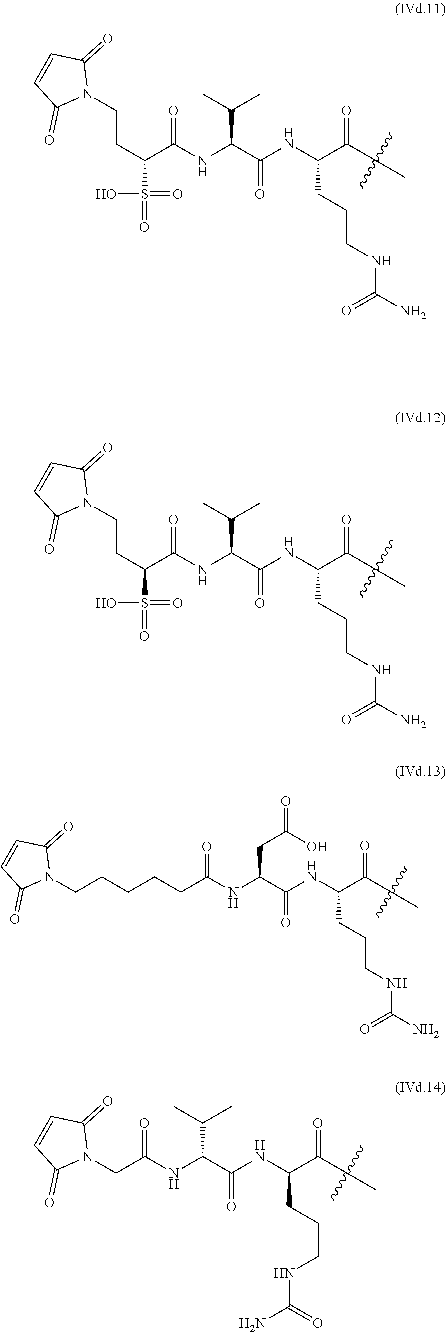 [SMILES: *C(=O)[C@H](CCCNC(N)=O)NC(=O)[C@@H](NC(=O)[C@@H](CCN1C(=O)C=CC1=O)S(=O)(=O)O)C(C)C ; *C(=O)[C@H](CCCNC(N)=O)NC(=O)[C@@H](NC(=O)[C@H](CCN1C(=O)C=CC1=O)S(=O)(=O)O)C(C)C ; *C(=O)[C@H](CCCNC(N)=O)NC(=O)[C@H](CC(=O)O)NC(=O)CCCCCN1C(=O)C=CC1=O ; *C(=O)[C@@H](CCCNC(N)=O)NC(=O)[C@H](NC(=O)CN1C(=O)C=CC1=O)C(C)C]

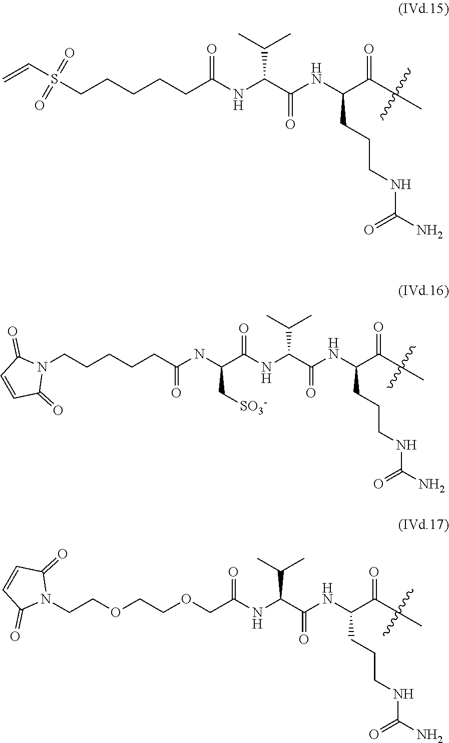

In certain embodiments, the ADC linker comprising structural formula (IVa), (IVb), (IVc), or (IVd) further comprises a carbonate moiety cleavable by exposure to an acidic medium. In particular embodiments, the ADC linker is attached through an oxygen to a cytotoxic and/or cytostatic agent.

### 7.8.2.2. Non-Cleavable Linkers

Although cleavable ADC linkers may provide certain advantages, the ADC linkers comprising the ADCs of the disclosure need not be cleavable. For noncleavable ADC linkers, the release of drug does not depend on the differential properties between the plasma and some cytoplasmic compartments. The release of the drug is postulated to occur after internalization of the ADC via antigen-mediated endocytosis and delivery to lysosomal compartment, where the MBM is degraded to the level of amino acids through intracellular proteolytic degradation. This process releases a drug derivative, which is formed by the drug, the ADC linker, and the amino acid residue to which the ADC linker was covalently attached. The amino acid drug metabolites from conjugates with noncleavable ADC linkers are more hydrophilic and generally less membrane permeable, which leads to less bystander effects and less nonspecific toxicities compared to conjugates with a cleavable ADC linker. In general, ADCs with noncleavable ADC linkers have greater stability in circulation than ADCs with cleavable ADC linkers. Non-cleavable ADC linkers may be alkylene chains, or maybe polymeric in natures, such as, for example, based upon polyalkylene glycol polymers, amide polymers, or may include segments of alkylene chains, polyalkylene glocols and/or amide polymers.

A variety of non-cleavable ADC linkers used to link drugs to MBMs have been described. See, Jeffrey et al., 2006, Bioconjug. Chem. 17; 831-840; Jeffrey et al., 2007, Bioorg. Med. Chem. Lett. 17:2278-2280; and Jiang et al., 2005, J. Am. Chem. Soc. 127:11254-11255. All of these ADC linkers may be included in the ADCs of the disclosure.

In certain embodiments, the ADC linker is non-cleavable in vivo, for example an ADC linker according to structural formula (VIa), (VIb), (VIc) or (VId) (as illustrated, the ADC linkers include a group suitable for covalently linking the ADC linker to a MBM:

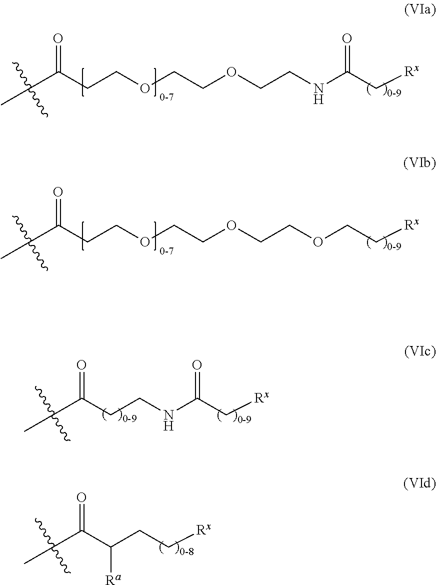

or salts thereof, where: R^{a} is selected from hydrogen, alkyl, sulfonate and methyl sulfonate; R^{x} is a moiety including a functional group capable of covalently linking the ADC linker to a MBM; and [symbol] represents the point of attachment of the ADC linker to a cytotoxic and/or cytostatic agent.

Specific exemplary embodiments of ADC linkers according to structural formula (VIa)-(VId) that may be included in the ADCs of the disclosure include the ADC linkers illustrated below (as illustrated, the ADC linkers include a group suitable for covalently linking the ADC linker to a MBM, and [symbol] represents the point of attachment to a cytotoxic and/or cytostatic agent):

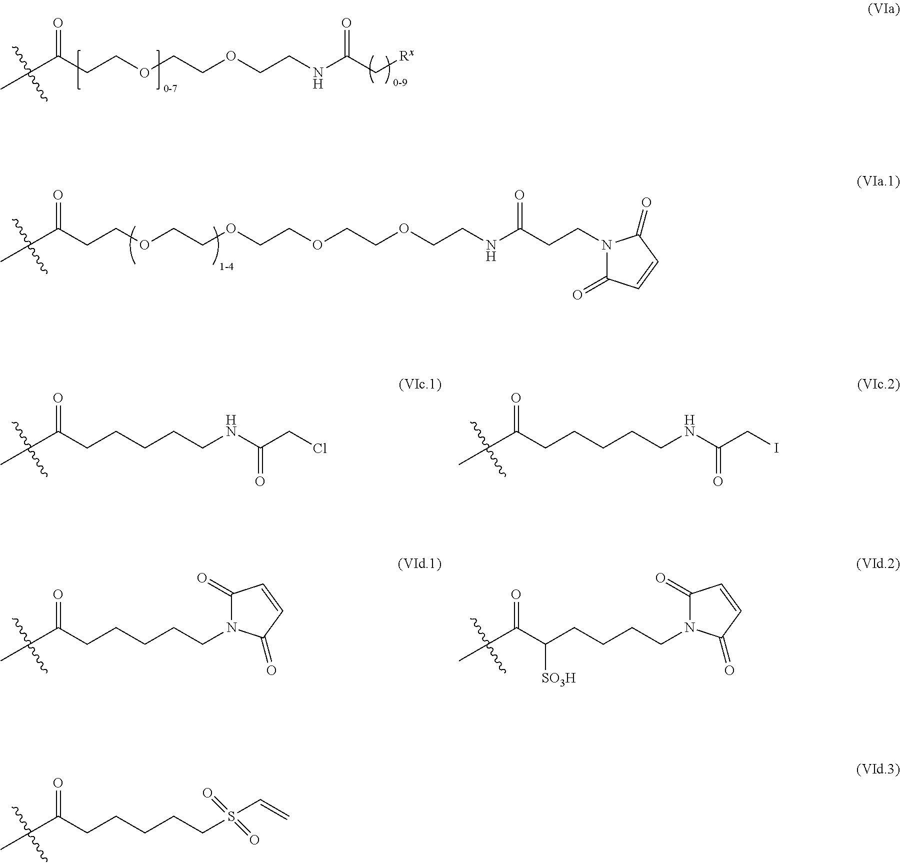

### 7.8.2.3. Groups Used to Attach Linkers to MBMs

A variety of groups may be used to attach ADC linker-drug synthons to MBMs (e.g., TBMs) to yield ADCs. Attachment groups can be electrophilic in nature and include: maleimide groups, activated disulfides, active esters such as NHS esters and HOBt esters, haloformates, acid halides, alkyl and benzyl halides such as haloacetamides. As discussed below, there are also emerging technologies related to “self-stabilizing” maleimides and “bridging disulfides” that can be used in accordance with the disclosure. The specific group used will depend, in part, on the site of attachment to the MBM.

One example of a “self-stabilizing” maleimide group that hydrolyzes spontaneously under MBM conjugation conditions to give an ADC species with improved stability is depicted in the schematic below. See US20130309256 A1; also Lyon et al., Nature Biotech published online, doi:10.1038/nbt.2968.

### Normal System:

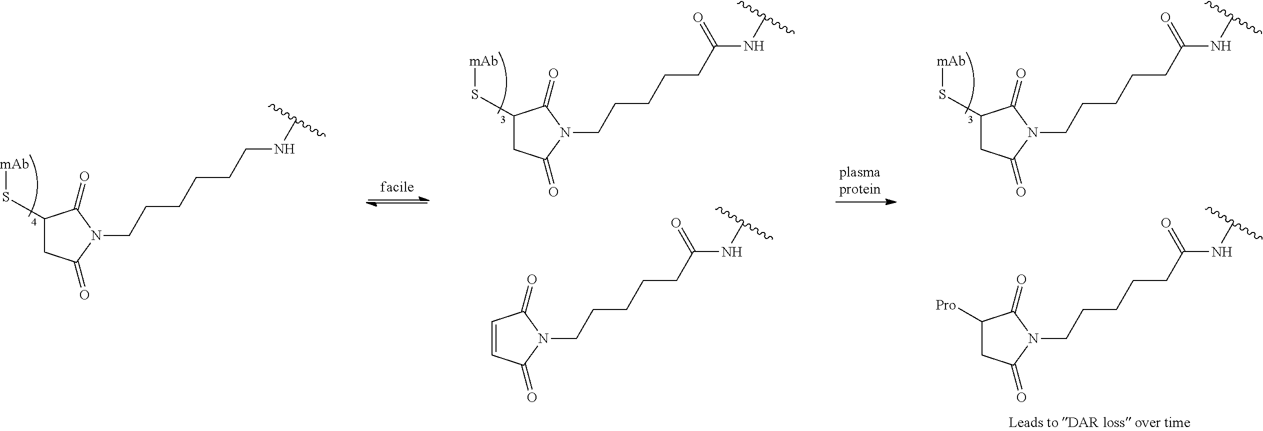

### SGN MalDPR (Maleimido Dipropylamino) System:

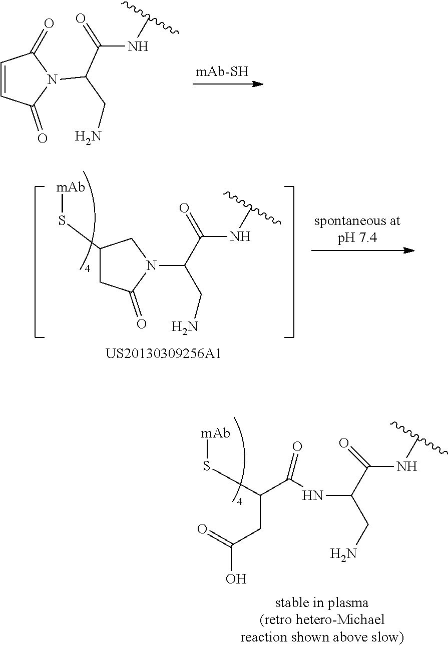

Polytherics has disclosed a method for bridging a pair of sulfhydryl groups derived from reduction of a native hinge disulfide bond. See, Badescu et al., 2014, Bioconjugate Chem. 25:1124-1136. The reaction is depicted in the schematic below. An advantage of this methodology is the ability to synthesize enriched DAR4 ADCs by full reduction of IgGs (to give 4 pairs of sulfhydryls) followed by reaction with 4 equivalents of the alkylating agent. ADCs containing “bridged disulfides” have increased stability.

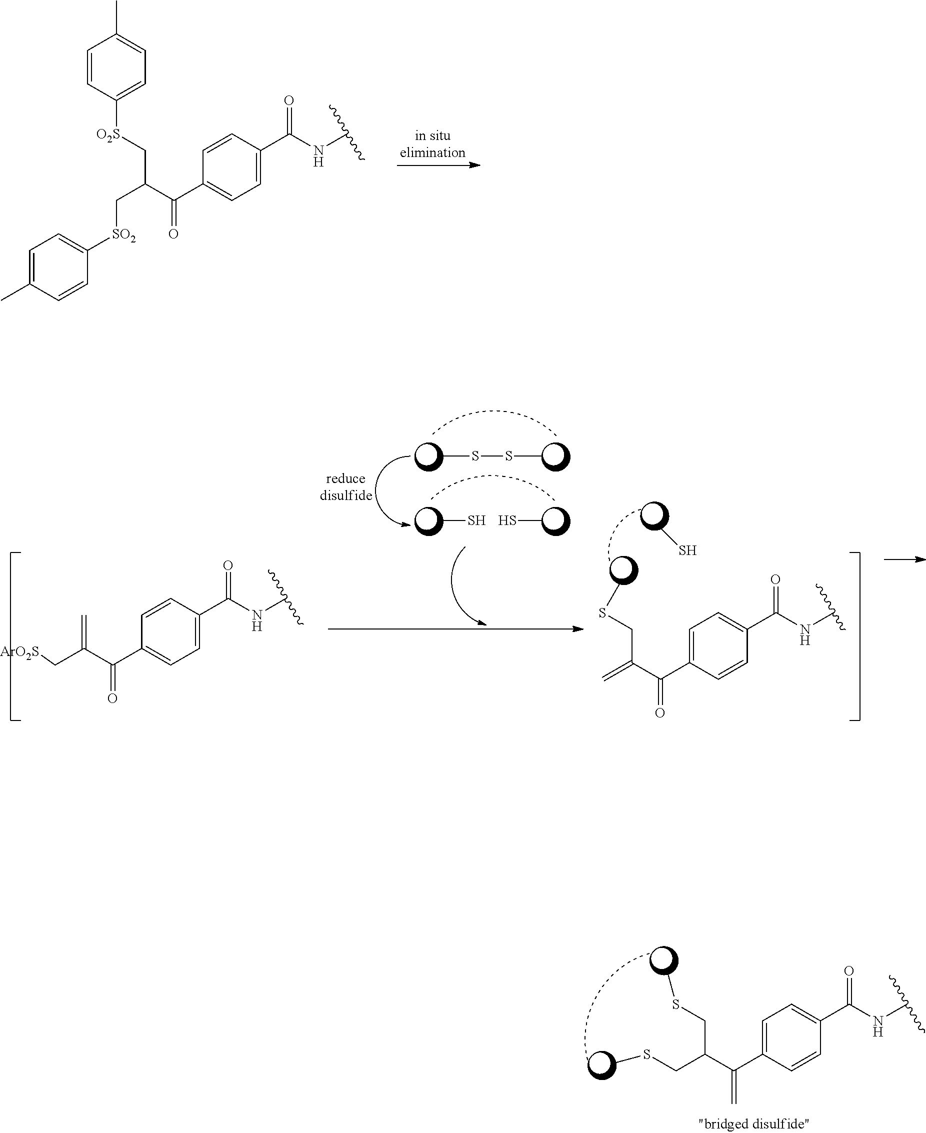

Similarly, as depicted below, a maleimide derivative (1, below) that is capable of bridging a pair of sulfhydryl groups has been developed. See WO2013/085925.

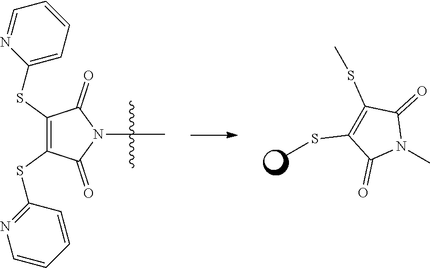

### 7.8.2.4. ADC Linker Selection Considerations

As is known by skilled artisans, the ADC linker selected for a particular ADC may be influenced by a variety of factors, including but not limited to, the site of attachment to the MBM (e.g., lys, cys or other amino acid residues), structural constraints of the drug pharmacophore and the lipophilicity of the drug. The specific ADC linker selected for an ADC should seek to balance these different factors for the specific MBM/drug combination. For a review of the factors that are influenced by choice of ADC linkers in ADCs, see Nolting, Chapter 5 “Linker Technology in Antibody-Drug Conjugates,” In: Antibody-Drug Conjugates: Methods in Molecular Biology, vol. 1045, pp. 71-100, Laurent Ducry (Ed.), Springer Science & Business Medica, LLC, 2013.

For example, ADCs have been observed to effect killing of bystander antigen-negative cells present in the vicinity of the antigen-positive tumor cells. The mechanism of bystander cell killing by ADCs has indicated that metabolic products formed during intracellular processing of the ADCs may play a role. Neutral cytotoxic metabolites generated by metabolism of the ADCs in antigen-positive cells appear to play a role in bystander cell killing while charged metabolites may be prevented from diffusing across the membrane into the medium and therefore cannot affect bystander killing. In certain embodiments, the ADC linker is selected to attenuate the bystander killing effect caused by cellular metabolites of the ADC. In certain embodiments, the ADC linker is selected to increase the bystander killing effect.

The properties of the ADC linker may also impact aggregation of the ADC under conditions of use and/or storage. Typically, ADCs reported in the literature contain no more than 3-4 drug molecules per antibody molecule (see, e.g., Chari, 2008, Acc Chem Res 41:98-107). Attempts to obtain higher drug-to-antibody ratios (“DAR”) often failed, particularly if both the drug and the ADC linker were hydrophobic, due to aggregation of the ADC (King et al., 2002, J Med Chem 45:4336-4343; Hollander et al., 2008, Bioconjugate Chem 19:358-361; Burke et al., 2009 Bioconjugate Chem 20:1242-1250). In many instances, DARs higher than 3-4 could be beneficial as a means of increasing potency. In instances where the cytotoxic and/or cytostatic agent is hydrophobic in nature, it may be desirable to select ADC linkers that are relatively hydrophilic as a means of reducing ADC aggregation, especially in instances where DARS greater than 3-4 are desired. Thus, in certain embodiments, the ADC linker incorporates chemical moieties that reduce aggregation of the ADCs during storage and/or use. An ADC linker may incorporate polar or hydrophilic groups such as charged groups or groups that become charged under physiological pH to reduce the aggregation of the ADCs. For example, an ADC linker may incorporate charged groups such as salts or groups that deprotonate, e.g., carboxylates, or protonate, e.g., amines, at physiological pH.

Exemplary polyvalent ADC linkers that have been reported to yield DARs as high as 20 that may be used to link numerous cytotoxic and/or cytostatic agents to a MBM are described in WO 2009/073445; WO 2010/068795; WO 2010/138719; WO 2011/120053; WO 2011/171020; WO 2013/096901; WO 2014/008375; WO 2014/093379; WO 2014/093394; WO 2014/093640.

In particular embodiments, the aggregation of the ADCs during storage or use is less than about 10% as determined by size-exclusion chromatography (SEC). In particular embodiments, the aggregation of the ADCs during storage or use is less than 10%, such as less than about 5%, less than about 4%, less than about 3%, less than about 2%, less than about 1%, less than about 0.5%, less than about 0.1%, or even lower, as determined by size-exclusion chromatography (SEC).

### 7.8.3. Methods of Making ADCs

The ADCs of the disclosure may be synthesized using chemistries that are well-known. The chemistries selected will depend upon, among other things, the identity of the cytotoxic and/or cytostatic agent(s), the ADC linker and the groups used to attach ADC linker to the MBM. Generally, ADCs according to formula (I) may be prepared according to the following scheme:

D-L-R^{x}+Ab-R^{y}→[D-L-XY]ₙ-Ab (I)

where D, L, Ab, XY and n are as previously defined, and R^{x} and R^{y} represent complementary groups capable of forming a covalent linkages with one another, as discussed above.

The identities of groups R^{x} and R^{y} will depend upon the chemistry used to link synthon D-L-R^{x} to the MBM. Generally, the chemistry used should not alter the integrity of the MBM, for example its ability to bind its target. In some cases, the binding properties of the conjugated antibody will closely resemble those of the unconjugated MBM. A variety of chemistries and techniques for conjugating molecules to biological molecules and in particular to immunoglobulins, whose components are typically building blocks of the MBMs of the disclosure, are well-known. See, e.g., Amon et al., “Monoclonal Antibodies For Immunotargeting Of Drugs In Cancer Therapy,” in: Monoclonal Antibodies And Cancer Therapy, Reisfeld et al. Eds., Alan R. Liss, Inc., 1985; Hellstrom et al., “Antibodies For Drug Delivery,” in: Controlled Drug Delivery, Robinson et al. Eds., Marcel Dekker, Inc., 2nd Ed. 1987 Thorpe, “Antibody Carriers Of Cytotoxic Agents In Cancer Therapy: A Review,” in: Monoclonal Antibodies '84: Biological And Clinical Applications, Pinchera et al., Eds., 1985; “Analysis, Results, and Future Prospective of the Therapeutic Use of Radiolabeled Antibody In Cancer Therapy,” in: Monoclonal Antibodies For Cancer Detection And Therapy, Baldwin et al., Eds., Academic Press, 1985; Thorpe et al., 1982, Immunol. Rev. 62:119-58; PCT publication WO 89/12624. Any of these chemistries may be used to link the synthons to a MBM.

A number of functional groups R^{x} and chemistries useful for linking synthons to accessible lysine residues are known, and include by way of example and not limitation NHS-esters and isothiocyanates.

A number of functional groups R^{x} and chemistries useful for linking synthons to accessible free sulfhydryl groups of cysteine residues are known, and include by way of example and not limitation haloacetyls and maleimides.

However, conjugation chemistries are not limited to available side chain groups. Side chains such as amines may be converted to other useful groups, such as hydroxyls, by linking an appropriate small molecule to the amine. This strategy can be used to increase the number of available linking sites on the antibody by conjugating multifunctional small molecules to side chains of accessible amino acid residues of the MBM. Functional groups R^{x} suitable for covalently linking the synthons to these “converted” functional groups are then included in the synthons.

The MBM may also be engineered to include amino acid residues for conjugation. An approach for engineering MBMs to include non-genetically encoded amino acid residues useful for conjugating drugs in the context of ADCs is described by Axup et al., 2012, Proc Natl Acad Sci USA. 109(40):16101-16106, as are chemistries and functional group useful for linking synthons to the non-encoded amino acids.

Typically, the synthons are linked to the side chains of amino acid residues of the MBM, including, for example, the primary amino group of accessible lysine residues or the sulfhydryl group of accessible cysteine residues. Free sulfhydryl groups may be obtained by reducing interchain disulfide bonds.

For linkages where R^{y} is a sulfhydryl group (for example, when R^{x} is a maleimide), the MBM is generally first fully or partially reduced to disrupt interchain disulfide bridges between cysteine residues.

Cysteine residues that do not participate in disulfide bridges may engineered into a MBM by modification of one or more codons. Reducing these unpaired cysteines yields a sulfhydryl group suitable for conjugation. In some embodiments, MBMs of the disclosure are engineered to introduce one or more cysteine residues as sites for conjugation to a drug moiety (see, Junutula, et al, 2008, Nat Biotechnol, 26:925-932).

Sites for cysteine substitution can be selected in a constant region to provide stable and homogeneous conjugates. A MBM of the disclosure can have, for example, two or more cysteine substitutions, and these substitutions can be used in combination with other modification and conjugation methods as described herein. Methods for inserting cysteine at specific locations of an antibody are known in the art, see, e.g., Lyons et al., 1990, Protein Eng., 3:703-708, WO 2011/005481, WO2014/124316, WO 2015/138615. In certain embodiments a MBM of the disclosure comprises a substitution of one or more amino acids with cysteine on a constant region selected from positions 117, 119, 121, 124, 139, 152, 153, 155, 157, 164, 169, 171, 174, 189, 205, 207, 246, 258, 269, 274, 286, 288, 290, 292, 293, 320, 322, 326, 333, 334, 335, 337, 344, 355, 360, 375, 382, 390, 392, 398, 400 and 422 of a heavy chain, where the positions are numbered according to the EU system. In some embodiments, a MBM comprises a substitution of one or more amino acids with cysteine on a constant region selected from positions 107, 108, 109, 114, 129, 142, 143, 145, 152, 154, 156, 159, 161, 165, 168, 169, 170, 182, 183, 197, 199, and 203 of a light chain, where the positions are numbered according to the EU system, and where the light chain is a human kappa light chain. In certain embodiments a MBM comprises a combination of substitution of two or more amino acids with cysteine on a constant region, where the combinations comprise substitutions at positions 375 of a heavy chain, position 152 of a heavy chain, position 360 of a heavy chain, or position 107 of a light chain and where the positions are numbered according to the EU system. In certain embodiments a MBM comprises a substitution of one amino acid with cysteine on a constant region where the substitution is position 375 of a heavy chain, position 152 of a heavy chain, position 360 of a heavy chain, position 107 of a light chain, position 165 of a light chain or position 159 of a light chain and where the positions are numbered according to the EU system, and where the light chain is a kappa chain.

In particular embodiments a MBM of the disclosure comprises a combination of substitution of two amino acids with cysteine on a constant regions, where the MBM comprises cysteines at positions 152 and 375 of a heavy chain, where the positions are numbered according to the EU system.

In other particular embodiments a MBM of the disclosure comprises a substitution of one amino acid with cysteine at position 360 of a heavy chain, where the positions are numbered according to the EU system.

In other particular embodiments a MBM of the disclosure comprises a substitution of one amino acid with cysteine at position 107 of a light chain, where the positions are numbered according to the EU system, and where the light chain is a kappa chain.

Other positions for incorporating engineered cysteines can include, by way of example and not limitation, positions S112C, S113C, A114C, S115C, A176C, S180C, S252C, V286C, V292C, S357C, A359C, S398C, S428C (Kabat numbering) on the human IgG₁ heavy chain and positions V110C, S114C, S121C, S127C, S168C, V205C (Kabat numbering) on the human Ig kappa light chain (see, e.g., U.S. Pat. Nos. 7,521,541, 7,855,275 and 8,455,622).

MBMs of the disclosure useful in ADCs disclosed herein the MBM can additionally or alternatively be modified to introduce one or more other reactive amino acids (other than cysteine), including Pcl, pyrrolysine, peptide tags (such as S6, A1 and ybbR tags), and non-natural amino acids, in place of at least one amino acid of the native sequence, thus providing a reactive site on the MBM for conjugation to a drug moiety. For example, MBMs can be modified to incorporate Pcl or pyrrolysine (W. Ou et al., 2011, PNAS, 108(26):10437-10442; WO2014124258) or unnatural amino acids (Axup, et al., 2012, PNAS, 109:16101-16106; for review, see C. C. Liu and P. G. Schultz, 2010, Annu Rev Biochem 79:413-444; Kim, et al., 2013, Curr Opin Chem Biol. 17:412-419) as sites for conjugation to a drug. Similarly, peptide tags for enzymatic conjugation methods can be introduced into a MBM (see, Strop et al. 2013, Chem Biol. 20(2):161-7; Rabuka, 2010, Curr Opin Chem Biol. 14(6):790-6; Rabuka, et al., 2012, Nat Protoc. 7(6):1052-67). One other example is the use of 4′-phosphopantetheinyl transferases (PPTase) for the conjugation of Coenzyme A analogs (WO2013184514). Such modified or engineered MBMs can be conjugated with payloads or linker-payload combinations according to methods known in the art.

As will appreciated by skilled artisans, the number of agents (e.g., cytotoxic and/or cytostatic agents) linked to a MBM molecule may vary, such that a collection of ADCs may be heterogeneous in nature, where some MBMs contain one linked agent, some two, some three, etc. (and some none). The degree of heterogeneity will depend upon, among other things, the chemistries used for linking the agents. For example, where the MBMs are reduced to yield sulfhydryl groups for attachment, heterogeneous mixtures of MBMs having zero, 2, 4, 6 or 8 linked agents per molecule are often produced. Furthermore, by limiting the molar ratio of attachment compound, MBMs having zero, 1, 2, 3, 4, 5, 6, 7 or 8 linked agents per molecule are often produced. Thus, it will be understood that depending upon context, stated drug MBM ratios (DTRs) may be averages for a collection of MBMs. For example, “DTR4” can refer to an ADC preparation that has not been subjected to purification to isolate specific DTR peaks and can comprise a heterogeneous mixture of ADC molecules having different numbers of cytostatic and/or cytotoxic agents attached per MBM (e.g., 0, 2, 4, 6, 8 agents per MBM), but has an average drug-to-MBM ratio of 4. Similarly, in some embodiments, “DTR2” refers to a heterogeneous ADC preparation in which the average drug-to-MBM ratio is 2.

When enriched preparations are desired, MBMs having defined numbers of linked agents (e.g., cytotoxic and/or cytostatic agents) may be obtained via purification of heterogeneous mixtures, for example, via column chromatography, e.g., hydrophobic interaction chromatography.

Purity may be assessed by a variety of methods, as is known in the art. As a specific example, an ADC preparation may be analyzed via HPLC or other chromatography and the purity assessed by analyzing areas under the curves of the resultant peaks.

### 7.9. Pharmaceutical Compositions

The MBMs of the disclosure (e.g., TBMs) (as well as their conjugates; references to MBMs in this disclosure also refers to conjugates comprising the MBMs, such as ADCs, unless the context dictates otherwise) can be formulated as pharmaceutical compositions comprising the MBMs, for example containing one or more pharmaceutically acceptable excipients or carriers. To prepare pharmaceutical or sterile compositions comprising the MBMs of the present disclosure a MBM preparation can be combined with one or more pharmaceutically acceptable excipient or carrier.

For example, formulations of MBMs can be prepared by mixing MBMs with physiologically acceptable carriers, excipients, or stabilizers in the form of, e.g., lyophilized powders, slurries, aqueous solutions, lotions, or suspensions (see, e.g., Hardman et al., 2001, Goodman and Gilman's The Pharmacological Basis of Therapeutics, McGraw-Hill, New York, N.Y.; Gennaro, 2000, Remington: The Science and Practice of Pharmacy, Lippincott, Williams, and Wilkins, New York, N.Y.; Avis, et al. (eds.), 1993, Pharmaceutical Dosage Forms: General Medications, Marcel Dekker, NY; Lieberman, et al. (eds.), 1990, Pharmaceutical Dosage Forms: Tablets, Marcel Dekker, NY; Lieberman, et al. (eds.), 1990, Pharmaceutical Dosage Forms: Disperse Systems, Marcel Dekker, NY; Weiner and Kotkoskie, 2000, Excipient Toxicity and Safety, Marcel Dekker, Inc., New York, N.Y.).

Selecting an administration regimen for a MBM depends on several factors, including the serum or tissue turnover rate of the MBM, the level of symptoms, the immunogenicity of the MBM, and the accessibility of the target cells. In certain embodiments, an administration regimen maximizes the amount of MBM delivered to the subject consistent with an acceptable level of side effects. Accordingly, the amount of MBM delivered depends in part on the particular MBM and the severity of the condition being treated. Guidance in selecting appropriate doses of antibodies and small molecules are available (see, e.g., Wawrzynczak, 1996, Antibody Therapy, Bios Scientific Pub. Ltd, Oxfordshire, UK; Kresina (ed.), 1991, Monoclonal Antibodies, Cytokines and Arthritis, Marcel Dekker, New York, N.Y.; Bach (ed.), 1993, Monoclonal Antibodies and Peptide Therapy in Autoimmune Diseases, Marcel Dekker, New York, N.Y.; Baert et al., 2003, New Engl. J. Med. 348:601-608; Milgrom et al., 1999, New Engl. J. Med. 341:1966-1973; Slamon et al., 2001, New Engl. J. Med. 344:783-792; Beniaminovitz et al., 2000, New Engl. J. Med. 342:613-619; Ghosh et al., 2003, New Engl. J. Med. 348:24-32; Lipsky et al., 2000, New Engl. J. Med. 343:1594-1602).

Determination of the appropriate dose is made by the clinician, e.g., using parameters or factors known or suspected in the art to affect treatment or predicted to affect treatment. Generally, the dose begins with an amount somewhat less than the optimum dose and it is increased by small increments thereafter until the desired or optimum effect is achieved relative to any negative side effects. Important diagnostic measures include those of symptoms of, e.g., the inflammation or level of inflammatory cytokines produced.

Actual dosage levels of the MBMs in the pharmaceutical compositions of the present disclosure may be varied so as to obtain an amount of the MBM which is effective to achieve the desired therapeutic response for a particular subject, composition, and mode of administration, without being toxic to the subject. The selected dosage level will depend upon a variety of pharmacokinetic factors including the activity of the particular MBM, the route of administration, the time of administration, the rate of excretion of the particular MBM being employed, the duration of the treatment, other agents (e.g., active agents such as therapeutic drugs or compounds and/or inert materials used as carriers) in combination with the particular MBM employed, the age, sex, weight, condition, general health and prior medical history of the subject being treated, and like factors known in the medical arts.

Compositions comprising the MBMs of the disclosure can be provided by continuous infusion, or by doses at intervals of, e.g., one day, one week, or 1-7 times per week. Doses can be provided intravenously, subcutaneously, topically, orally, nasally, rectally, intramuscular, intracerebrally, or by inhalation. A specific dose protocol is one involving the maximal dose or dose frequency that avoids significant undesirable side effects.

An effective amount for a particular subject may vary depending on factors such as the condition being treated, the overall health of the subject, the method route and dose of administration and the severity of side effects (see, e.g., Maynard, et al. (1996) A Handbook of SOPs for Good Clinical Practice, Interpharm Press, Boca Raton, Fla.; Dent (2001) Good Laboratory and Good Clinical Practice, Urch Publ., London, UK).

The route of administration may be by, e.g., topical or cutaneous application, injection or infusion by intravenous, intraperitoneal, intracerebral, intramuscular, intraocular, intraarterial, intracerebrospinal, intralesional, or by sustained release systems or an implant (see, e.g., Sidman et al., 1983, Biopolymers 22:547-556; Langer et al., 1981, J. Biomed. Mater. Res. 15:167-277; Langer, 1982, Chem. Tech. 12:98-105; Epstein et al., 1985, Proc. Natl. Acad. Sci. USA 82:3688-3692; Hwang et al., 1980, Proc. Natl. Acad. Sci. USA 77:4030-4034; U.S. Pat. Nos. 6,350,466 and 6,316,024). Where necessary, the composition may also include a solubilizing agent and a local anesthetic such as lidocaine to ease pain at the site of the injection. In addition, pulmonary administration can also be employed, e.g., by use of an inhaler or nebulizer, and formulation with an aerosolizing agent. See, e.g., U.S. Pat. Nos. 6,019,968, 5,985,320, 5,985,309, 5,934,272, 5,874,064, 5,855,913, 5,290,540, and 4,880,078; and PCT Publication Nos. WO 92/19244, WO 97/32572, WO 97/44013, WO 98/31346, and WO 99/66903.

A composition of the present disclosure may also be administered via one or more routes of administration using one or more of a variety of methods known in the art. As will be appreciated by the skilled artisan, the route and/or mode of administration will vary depending upon the desired results. Selected routes of administration for MBMs include intravenous, intramuscular, intradermal, intraperitoneal, subcutaneous, spinal or other general routes of administration, for example by injection or infusion. General administration may represent modes of administration other than enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intraarticular, subcapsular, subarachnoid, intraspinal, epidural and intrasternal injection and infusion. Alternatively, a composition of the disclosure can be administered via a non-general route, such as a topical, epidermal or mucosal route of administration, for example, intranasally, orally, vaginally, rectally, sublingually or topically. In one embodiment, the MBMs is administered by infusion. In another embodiment, the multispecific epitope binding protein of the disclosure is administered subcutaneously.

If the MBMs are administered in a controlled release or sustained release system, a pump may be used to achieve controlled or sustained release (see Langer, supra; Sefton, 1987, CRC Crit. Ref Biomed. Eng. 14:20; Buchwald et al., 1980, Surgery 88:507; Saudek et al., 1989, N. Engl. J. Med. 321:574). Polymeric materials can be used to achieve controlled or sustained release of the therapies of the disclosure (see, e.g., Medical Applications of Controlled Release, Langer and Wise (eds.), CRC Pres., Boca Raton, Fla. (1974); Controlled Drug Bioavailability, Drug Product Design and Performance, Smolen and Ball (eds.), Wiley, New York (1984); Ranger and Peppas, 1983, J., Macromol. Sci. Rev. Macromol. Chem. 23:61; see also Levy et al., 1985, Science 228:190; During et al., 1989, Ann. Neurol. 25:351; Howard et al., 1989, J. Neurosurg. 71:105); U.S. Pat. Nos. 5,679,377; 5,916,597; 5,912,015; 5,989,463; 5,128,326; PCT Publication No. WO 99/15154; and PCT Publication No. WO 99/20253. Examples of polymers used in sustained release formulations include, but are not limited to, poly(2-hydroxy ethyl methacrylate), poly(methyl methacrylate), poly(acrylic acid), poly(ethylene-co-vinyl acetate), poly(methacrylic acid), polyglycolides (PLG), polyanhydrides, poly(N-vinyl pyrrolidone), poly(vinyl alcohol), polyacrylamide, poly(ethylene glycol), polylactides (PLA), poly(lactide-co-glycolides) (PLGA), and polyorthoesters. In one embodiment, the polymer used in a sustained release formulation is inert, free of leachable impurities, stable on storage, sterile, and biodegradable. A controlled or sustained release system can be placed in proximity of the prophylactic or therapeutic target, thus requiring only a fraction of the systemic dose (see, e.g., Goodson, in Medical Applications of Controlled Release, supra, vol. 2, pp. 115-138 (1984)).

Controlled release systems are discussed in the review by Langer (1990, Science 249:1527-1533). Any technique known to one of skill in the art can be used to produce sustained release formulations comprising one or more MBMs of the disclosure. See, e.g., U.S. Pat. No. 4,526,938, PCT publication WO 91/05548, PCT publication WO 96/20698, Ning et al., 1996, Radiotherapy & Oncology 39:179-189, Song et al., 1995, PDA Journal of Pharmaceutical Science & Technology 50:372-397, Cleek et al., 1997, Pro. Int'l. Symp. Control. Rel. Bioact. Mater. 24:853-854, and Lam et al., 1997, Proc. Int'l. Symp. Control Rel. Bioact. Mater. 24:759-760.

If the MBMs are administered topically, they can be formulated in the form of an ointment, cream, transdermal patch, lotion, gel, shampoo, spray, aerosol, solution, emulsion, or other form well-known to one of skill in the art. See, e.g., Remington's Pharmaceutical Sciences and Introduction to Pharmaceutical Dosage Forms, 19th ed., Mack Pub. Co., Easton, Pa. (1995). For non-sprayable topical dosage forms, viscous to semi-solid or solid forms comprising a carrier or one or more excipients compatible with topical application and having a dynamic viscosity, in some instances, greater than water are typically employed. Suitable formulations include, without limitation, solutions, suspensions, emulsions, creams, ointments, powders, liniments, salves, and the like, which are, if desired, sterilized or mixed with auxiliary agents (e.g., preservatives, stabilizers, wetting agents, buffers, or salts) for influencing various properties, such as, for example, osmotic pressure. Other suitable topical dosage forms include sprayable aerosol preparations where the active ingredient, in some instances, in combination with a solid or liquid inert carrier, is packaged in a mixture with a pressurized volatile (e.g., a gaseous propellant, such as freon) or in a squeeze bottle. Moisturizers or humectants can also be added to pharmaceutical compositions and dosage forms if desired. Examples of such additional ingredients are well-known in the art.

If the compositions comprising the MBMs are administered intranasally, the MBMs can be formulated in an aerosol form, spray, mist or in the form of drops. In particular, prophylactic or therapeutic agents for use according to the present disclosure can be conveniently delivered in the form of an aerosol spray presentation from pressurized packs or a nebulizer, with the use of a suitable propellant (e.g., dichlorodifluoromethane, trichlorofluoromethane, dichlorotetrafluoroethane, carbon dioxide or other suitable gas). In the case of a pressurized aerosol the dosage unit may be determined by providing a valve to deliver a metered amount. Capsules and cartridges (composed of, e.g., gelatin) for use in an inhaler or insufflator may be formulated containing a powder mix of the compound and a suitable powder base such as lactose or starch.

The MBMs (e.g., TBMs) of the disclosure can be administered in combination therapy regimens, as described in Section 7.11, infra.

In certain embodiments, the MBMs can be formulated to ensure proper distribution in vivo. For example, the blood-brain barrier (BBB) excludes many highly hydrophilic compounds. To ensure that the therapeutic compounds of the disclosure cross the BBB (if desired), they can be formulated, for example, in liposomes. For methods of manufacturing liposomes, see, e.g., U.S. Pat. Nos. 4,522,811; 5,374,548; and 5,399,331. The liposomes may comprise one or more moieties which are selectively transported into specific cells or organs, thus enhance targeted drug delivery (see, e.g., Ranade, 1989, J. Clin. Pharmacol. 29:685). Exemplary targeting moieties include folate or biotin (see, e.g., U.S. Pat. No. 5,416,016 to Low et al.); mannosides (Umezawa et al., 1988, Biochem. Biophys. Res. Commun. 153:1038); antibodies (Bloeman et al., 1995, FEBS Lett. 357:140; Owais et al., 1995, Antimicrob. Agents Chemother. 39:180); surfactant protein A receptor (Briscoe et al., 1995, Am. J. Physiol. 1233:134); p 120 (Schreier et al., 1994, J. Biol. Chem. 269:9090); see also Keinanen and Laukkanen, 1994, FEBS Lett. 346:123; Killion and Fidler, 1994, Immunomethods 4:273.

When used in combination therapy, e.g., as described in Section 7.11, infra, a MBM of the disclosure and one or more additional agents can be administered to a subject in the same pharmaceutical composition. Alternatively, the MBM and the additional agent(s) of the combination therapies can be administered concurrently to a subject in separate pharmaceutical compositions.

The therapeutic methods described herein may further comprise carrying out a “companion diagnostic” test whereby a sample from a subject who is a candidate for therapy with a MBM of the disclosure is tested for the expression of the TAA targeted by ABM1 or ABM2. The companion diagnostic test can be performed prior to initiating therapy with a MBM of the disclosure and/or during a therapeutic regimen with a MBM of the disclosure to monitor the subject's continued suitability for MBM therapy. The agent used in the companion diagnostic can be the MBM itself or another diagnostic agent, for example a labeled monospecific antibody against the TAA recognized by ABM1 or ABM2 or a nucleic acid probe to detect TAA RNA. The sample that can be tested in a companion diagnostic assay can be any sample in which the cells targeted by the MBM may be present, from example a tumor (e.g., a solid tumor) biopsy, lymph, stool, urine, blood or any other bodily fluid that might contain circulating tumor cells.

### 7.10. Therapeutic Indications

### 7.10.1. Cancer

The MBMs (e.g., TBMs) of the disclosure can be used in the treatment of any proliferative disorder (e.g., cancer) that expresses a TAA described in Section 7.6 or combination of TAAs described in Section 7.6 (e.g., a cancer characterized by cancerous cells expressing two TAAs on the same cancerous cell or a cancer characterized by cancerous cells expressing a first TAA and a second TAA on different cancerous cells). In specific embodiments, the cancer is a B cell malignancy. Exemplary types of B cell malignancies that may be targeted include Hodgkin's lymphomas, non-Hodgkin's lymphomas (NHLs), and multiple myeloma. Examples of NHLs include diffuse large B-cell lymphoma (DLBCL), follicular lymphoma, chronic lymphocytic leukemia (CLL)/small lymphocytic lymphoma (SLL), mantle cell lymphoma (MCL), marginal zone lymphomas, Burkitt lymphoma, lymphoplasmacytic lymphoma (Waldenstrom macroglobulinemia), hairy cell leukemia, primary central nervous system (CNS) lymphoma, primary mediastinal large B-cell lymphoma, mediastinal grey-zone lymphoma (MGZL), splenic marginal zone B-cell lymphoma, extranodal marginal zone B-cell lymphoma of MALT, nodal marginal zone B-cell lymphoma, and primary effusion lymphoma.

In some embodiments, the MBMs of the disclosure are used to treat Hodgkin's lymphoma.

In some embodiments, the MBMs of the disclosure are used to treat non-Hodgkin's lymphoma.

In some embodiments, the MBMs of the disclosure are used to treat diffuse large B-cell lymphoma (DLBCL).

In some embodiments, the MBMs of the disclosure are used to treat follicular lymphoma.

In some embodiments, the MBMs of the disclosure are used to treat chronic lymphocytic leukemia (CLL)/small lymphocytic lymphoma (SLL).

In some embodiments, the MBMs of the disclosure are used to treat mantle cell lymphoma (MCL).

In some embodiments, the MBMs of the disclosure are used to treat marginal zone lymphoma.

In some embodiments, the MBMs of the disclosure are used to treat Burkitt lymphoma.

In some embodiments, the MBMs of the disclosure are used to treat lymphoplasmacytic lymphoma (Waldenstrom macroglobulinemia).

In some embodiments, the MBMs of the disclosure are used to treat hairy cell leukemia.

In some embodiments, the MBMs of the disclosure are used to treat primary central nervous system (CNS) lymphoma.

In some embodiments, the MBMs of the disclosure are used to treat primary mediastinal large B-cell lymphoma.

In some embodiments, the MBMs of the disclosure are used to treat mediastinal grey-zone lymphoma (MGZL).

In some embodiments, the MBMs of the disclosure are used to treat splenic marginal zone B-cell lymphoma.

In some embodiments, the MBMs of the disclosure are used to treat extranodal marginal zone B-cell lymphoma of MALT.

In some embodiments, the MBMs of the disclosure are used to treat nodal marginal zone B-cell lymphoma.

In some embodiments, the MBMs of the disclosure are used to treat primary effusion lymphoma.

In some embodiments, the MBMs of the disclosure are used to treat a plasmacytic dendritic cell neoplasm.

In some embodiments, the MBMs of the disclosure are used to treat multiple myeloma.

### 7.10.2. Autoimmune Disorders

The MBMs (e.g., TBMs) of the disclosure can be used in the treatment of autoimmune disorders, which can result from the loss of B-cell tolerance and the inappropriate production of autoantibodies. Autoimmune disorders that can be treated with the MBMs of the disclosure include systemic lupus erythematosus (SLE), Sjögren's syndrome, scleroderma, rheumatoid arthritis (RA), juvenile idiopathic arthritis, graft versus host disease, dermatomyositis, type I diabetes mellitus, Hashimoto's thyroiditis, Graves's disease, Addison's disease, celiac disease, Crohn's Disease, pernicious anaemia, pemphigus vulgaris, vitiligo, autoimmune haemolytic anaemia, idiopathic thrombocytopenic purpura, giant cell arteritis, myasthenia gravis, multiple sclerosis (MS) (e.g., relapsing-remitting MS (RRMS)), glomerulonephritis, Goodpasture's syndrome, bullous pemphigoid, colitis ulcerosa, Guillain-Barré syndrome, chronic inflammatory demyelinating polyneuropathy, anti-phospholipid syndrome, narcolepsy, sarcoidosis, and Wegener's granulomatosis.

In some embodiments, the MBMs of the disclosure are used to treat systemic lupus erythematosus (SLE).

In some embodiments, the MBMs of the disclosure are used to treat Sjögren's syndrome.

In some embodiments, the MBMs of the disclosure are used to treat scleroderma.

In some embodiments, the MBMs of the disclosure are used to treat rheumatoid arthritis (RA).

In some embodiments, the MBMs of the disclosure are used to treat juvenile idiopathic arthritis.

In some embodiments, the MBMs of the disclosure are used to treat graft versus host disease.

In some embodiments, the MBMs of the disclosure are used to treat dermatomyositis.

In some embodiments, the MBMs of the disclosure are used to treat type I diabetes mellitus.

In some embodiments, the MBMs of the disclosure are used to treat Hashimoto's thyroiditis.

In some embodiments, the MBMs of the disclosure are used to treat Graves's disease.

In some embodiments, the MBMs of the disclosure are used to treat Addison's disease.

In some embodiments, the MBMs of the disclosure are used to treat celiac disease.

In some embodiments, the MBMs of the disclosure are used to treat Crohn's Disease.

In some embodiments, the MBMs of the disclosure are used to treat pernicious anaemia.

In some embodiments, the MBMs of the disclosure are used to treat pemphigus vulgaris.

In some embodiments, the MBMs of the disclosure are used to treat vitiligo.

In some embodiments, the MBMs of the disclosure are used to treat autoimmune haemolytic anaemia.

In some embodiments, the MBMs of the disclosure are used to treat idiopathic thrombocytopenic purpura.

In some embodiments, the MBMs of the disclosure are used to treat giant cell arteritis.

In some embodiments, the MBMs of the disclosure are used to treat myasthenia gravis.

In some embodiments, the MBMs of the disclosure are used to treat multiple sclerosis (MS). In some embodiments, the MS is relapsing-remitting MS (RRMS).

In some embodiments, the MBMs of the disclosure are used to treat glomerulonephritis.

In some embodiments, the MBMs of the disclosure are used to treat Goodpasture's syndrome.

In some embodiments, the MBMs of the disclosure are used to treat bullous pemphigoid.

In some embodiments, the MBMs of the disclosure are used to treat colitis ulcerosa.

In some embodiments, the MBMs of the disclosure are used to treat Guillain-Barré syndrome.

In some embodiments, the MBMs of the disclosure are used to treat chronic inflammatory demyelinating polyneuropathy.

In some embodiments, the MBMs of the disclosure are used to treat anti-phospholipid syndrome.

In some embodiments, the MBMs of the disclosure are used to treat narcolepsy.

In some embodiments, the MBMs of the disclosure are used to treat sarcoidosis.

In some embodiments, the MBMs of the disclosure are used to treat Wegener's granulomatosis.

### 7.11. Combination Therapy

A MBM (e.g., a TBM) of the disclosure may be used in combination with other known agents and therapies. For example, the MBMs of the disclosure can be used in treatment regimens in combination with surgery, chemotherapy, antibodies, radiation, peptide vaccines, steroids, cytoxins, proteasome inhibitors, immunomodulatory drugs (e.g., IMiDs), BH3 mimetics cytokine therapies, stem cell transplant or a combination thereof. Without being bound by theory, it is believed that one of the advantages of the MBMs of the disclosure is that they can circumvent the need for administering separate antibodies to a subject suffering from a B cell malignancy. Accordingly, in certain embodiments, the one or more additional agents do not include an antibody (e.g., rituximab).

For convenience, an agent that is used in combination with a MBM of the disclosure is referred to herein as an “additional” agent.

Administered “in combination,” as used herein, means that two (or more) different treatments are delivered to the subject during the course of the subject's affliction with the disorder, e.g., the two or more treatments are delivered after the subject has been diagnosed with the disorder and before the disorder has been cured or eliminated or treatment has ceased for other reasons. In some embodiments, the delivery of one treatment is still occurring when the delivery of the second begins, so that there is overlap in terms of administration. This is sometimes referred to herein as “simultaneous” or “concurrent delivery”. The term “concurrently” is not limited to the administration of therapies (e.g., a MBM and an additional agent) at exactly the same time, but rather it is meant that a pharmaceutical composition comprising a MBM of the disclosure is administered to a subject in a sequence and within a time interval such that the MBMs of the disclosure can act together with the additional therapy(ies) to provide an increased benefit than if they were administered otherwise. For example, each therapy may be administered to a subject at the same time or sequentially in any order at different points in time; however, if not administered at the same time, they should be administered sufficiently close in time so as to provide the desired therapeutic effect.

A MBM of the disclosure and one or more additional agents can be administered simultaneously, in the same or in separate compositions, or sequentially. For sequential administration, the MBM can be administered first, and the additional agent can be administered second, or the order of administration can be reversed.

The MBM and the additional agent(s) can be administered to a subject in any appropriate form and by any suitable route. In some embodiments, the routes of administration are the same. In other embodiments the routes of administration are different.

In other embodiments, the delivery of one treatment ends before the delivery of the other treatment begins.

In some embodiments of either case, the treatment is more effective because of combined administration. For example, the second treatment is more effective, e.g., an equivalent effect is seen with less of the second treatment, or the second treatment reduces symptoms to a greater extent, than would be seen if the second treatment were administered in the absence of the first treatment, or the analogous situation is seen with the first treatment. In some embodiments, delivery is such that the reduction in a symptom, or other parameter related to the disorder is greater than what would be observed with one treatment delivered in the absence of the other. The effect of the two treatments can be partially additive, wholly additive, or greater than additive. The delivery can be such that an effect of the first treatment delivered is still detectable when the second is delivered.

The MBMs of the disclosure and/or additional agents can be administered during periods of active disorder, or during a period of remission or less active disease. A MBM can be administered before the treatment with the additional agent(s), concurrently with the treatment with the additional agent(s), post-treatment with the additional agent(s), or during remission of the disorder.

When administered in combination, the MBM and/or the additional agent(s) can be administered in an amount or dose that is higher, lower or the same than the amount or dosage of each agent used individually, e.g., as a monotherapy.

The additional agent(s) of the combination therapies of the disclosure can be administered to a subject concurrently. The term “concurrently” is not limited to the administration of therapies (e.g., prophylactic or therapeutic agents) at exactly the same time, but rather it is meant that a pharmaceutical composition comprising a MBM of the disclosure is administered to a subject in a sequence and within a time interval such that the molecules of the disclosure can act together with the additional therapy(ies) to provide an increased benefit than if they were administered otherwise. For example, each therapy may be administered to a subject at the same time or sequentially in any order at different points in time; however, if not administered at the same time, they should be administered sufficiently close in time so as to provide the desired therapeutic or prophylactic effect. Each therapy can be administered to a subject separately, in any appropriate form and by any suitable route.

The MBM and the additional agent(s) may be administered to a subject by the same or different routes of administration.

The MBMs and the additional agent(s) may be cyclically administered. Cycling therapy involves the administration of a first therapy (e.g., a first prophylactic or therapeutic agent) for a period of time, followed by the administration of a second therapy (e.g., a second prophylactic or therapeutic agent) for a period of time, optionally, followed by the administration of a third therapy (e.g., prophylactic or therapeutic agent) for a period of time and so forth, and repeating this sequential administration, i.e., the cycle in order to reduce the development of resistance to one of the therapies, to avoid or reduce the side effects of one of the therapies, and/or to improve the efficacy of the therapies.

In certain instances, the one or more additional agents, are other anti-cancer agents, anti-allergic agents, anti-nausea agents (or anti-emetics), pain relievers, cytoprotective agents, and combinations thereof.

In one embodiment, a MBM of the disclosure is administered in combination with an anti-cancer agent. Anti-cancer agents of particular interest for combinations with the MBMs of the present disclosure include: anthracyclines; alkylating agents; antimetabolites; drugs that inhibit either the calcium dependent phosphatase calcineurin or the p70S6 kinase FK506) or inhibit the p70S6 kinase; mTOR inhibitors; immunomodulators; anthracyclines; vinca alkaloids; proteasome inhibitors; GITR agonists; protein tyrosine phosphatase inhibitors; a CDK4 kinase inhibitor; a BTK inhibitor; a MKN kinase inhibitor; a DGK kinase inhibitor; an oncolytic virus; a BH3 mimetic, and cytokine therapies.

A MBM of the disclosure can be administered in combination with one or more anti-cancer agents that prevent or slow shedding of an antigen targeted by one or more of the ABMs of the MBM, thereby reducing the amount of soluble TAA and/or increasing the amount of cell surface bound TAA. For example, MBMs can be administered in combination with an ADAM10/17 inhibitor (e.g., INCB7839), e.g., to block shedding of an antigen released from cancer a cell by ADAM10/17, or in combination with a phospholipase inhibitor, e.g., to block shedding of an antigen released from a cancer cell by a phospholipase. Also of particular interest for combinations with the MBMs of the present disclosure that have an ABM targeting BCMA are gamma secretase modulators such as gamma secretase inhibitors (GSIs).

Exemplary alkylating agents include, without limitation, nitrogen mustards, ethylenimine derivatives, alkyl sulfonates, nitrosoureas and triazenes): uracil mustard (Aminouracil Mustard®, Chlorethaminacil®, Demethyldopan®, Desmethyldopan®, Haemanthamine®, Nordopan®, Uracillost®, Uracilmostaza®, Uramustin®, Uramustine®), chlormethine (Mustargen®), cyclophosphamide (Cytoxan®, Neosar®, Clafen®, Endoxan®, Procytox®, Revimmune™), ifosfamide (Mitoxana®), melphalan (Alkeran®), Chlorambucil (Leukeran®), pipobroman (Amedel®, Vercyte®), triethylenemelamine (Hemel®, Hexalen®, Hexastat®), triethylenethiophosphoramine, Temozolomide (Temodar®), thiotepa (Thioplex®), busulfan (Busilvex®, Myleran®), carmustine (BiCNU®), lomustine (CeeNU®), streptozocin (Zanosar®), and Dacarbazine (DTIC-Dome®). Additional exemplary alkylating agents include, without limitation, Oxaliplatin (Eloxatin®); Temozolomide (Temodar® and Temodal®); Dactinomycin (also known as actinomycin-D, Cosmegen®); Melphalan (also known as L-PAM, L-sarcolysin, and phenylalanine mustard, Alkeran®); Altretamine (also known as hexamethylmelamine (HMM), Hexalen®); Carmustine (BiCNU®); Bendamustine (Treanda®); Busulfan (Busulfex® and Myleran®); Carboplatin (Paraplatin®); Lomustine (also known as CCNU, CeeNU®); Cisplatin (also known as CDDP, Platinol® and Platinol®-AQ); Chlorambucil (Leukeran®); Cyclophosphamide (Cytoxan® and Neosar®); Dacarbazine (also known as DTIC, DIC and imidazole carboxamide, DTIC-Dome®); Altretamine (also known as hexamethylmelamine (HMM), Hexalen®); Ifosfamide (Ifex®); Prednumustine; Procarbazine (Matulane®); Mechlorethamine (also known as nitrogen mustard, mustine and mechloroethamine hydrochloride, Mustargen®); Streptozocin (Zanosar®); Thiotepa (also known as thiophosphoamide, TESPA and TSPA, Thioplex®); Cyclophosphamide (Endoxan®, Cytoxan®, Neosar®, Procytox®, Revimmune®); and Bendamustine HCl (Treanda®).

Exemplary mTOR inhibitors include, e.g., temsirolimus; ridaforolimus (formally known as deferolimus, (1R,2R,4S)-4-[(2R)-2 [(1R,9S,12S,15R,16E,18R,19R,21R, 23S,24E,26E,28Z,30S,32S,35R)-1,18-dihydroxy-19,30-dimethoxy-15,17,21,23, 29,35-hexamethyl-2,3,10,14,20-pentaoxo-11,36-dioxa-4-azatricyclo[30.3.1.04,9]hexatriaconta-16,24,26,28-tetraen-12-yl]propyl]-2-methoxycyclohexyl dimethylphosphinate, also known as AP23573 and MK8669, and described in PCT Publication No. WO 03/064383); everolimus (Afinitor® or RAD001); rapamycin (AY22989, sirolimus); simapimod (CAS 164301-51-3); emsirolimus, (5-{2,4-Bis[(3S)-3-methylmorpholin-4-yl]pyrido[2,3-d]pyrimidin-7-yl}-2-methoxyphenyl)methanol (AZD8055); 2-Amino-8-[trans-4-(2-hydroxyethoxy)cyclohexyl]-6-(6-methoxy-3-pyridinyl)-4-methyl-pyrido[2,3-d]pyrimidin-7(8H)-one (PF04691502, CAS 1013101-36-4); and N2-[1,4-dioxo-4-[[4-(4-oxo-8-phenyl-4H-1-benzopyran-2-yl)morpholinium-4-yl]methoxy]butyl]-L-arginylglycyl-L-α-aspartylL-serine-(SEQ ID NO: 727), inner salt (SF1126, CAS 936487-67-1), and XL765.

Exemplary immunomodulators include, e.g., afutuzumab (available from Roche®); pegfilgrastim (Neulasta®); lenalidomide (CC-5013, Revlimid®); IMIDs (such as thalidomide (Thalomid®), lenalidomide, pomalidomide, and apremilast), actimid (CC4047); and IRX-2 (mixture of human cytokines including interleukin 1, interleukin 2, and interferon γ, CAS 951209-71-5, available from IRX Therapeutics).

Exemplary anthracyclines include, e.g., doxorubicin (Adriamycin® and Rubex®); bleomycin (Ienoxane®); daunorubicin (dauorubicin hydrochloride, daunomycin, and rubidomycin hydrochloride, Cerubidine®); daunorubicin liposomal (daunorubicin citrate liposome, DaunoXome®); mitoxantrone (DHAD, Novantrone®); epirubicin (Ellence™); idarubicin (Idamycin®, Idamycin PFS®); mitomycin C (Mutamycin®); geldanamycin; herbimycin; ravidomycin; and desacetylravidomycin.

Exemplary vinca alkaloids include, e.g., vinorelbine tartrate (Navelbine®), Vincristine (Oncovin®), and Vindesine (Eldisine®)); vinblastine (also known as vinblastine sulfate, vincaleukoblastine and VLB, Alkaban-AQ® and Velban®); and vinorelbine (Navelbine®).

Exemplary proteasome inhibitors include bortezomib (Velcade®); carfilzomib (PX-171-007, (S)-4-Methyl-N—((S)-1-(((S)-4-methyl-1-((R)-2-methyloxiran-2-yl)-1-oxopentan-2-yl)amino)-1-oxo-3-phenylpropan-2-yl)-2-((S)-2-(2-morpholinoacetamido)-4-phenylbutanamido)-pentanamide); marizomib (NPI-0052); ixazomib citrate (MLN-9708); delanzomib (CEP-18770); and O-Methyl-N-[(2-methyl-5-thiazolyl)carbonyl]-L-seryl-O-methyl-N-[(1S)-2-[(2R)-2-methyl-2-oxiranyl]-2-oxo-1-(phenylmethyl)ethyl]-L-serinamide (ONX-0912).

Exemplary BH3 mimetics include venetoclax, ABT-737 (4-{4-[(4′-Chloro-2-biphenylyl)methyl]-1-piperazinyl}-N-[(4-{[(2R)-4-(dimethylamino)-1-(phenylsulfanyl)-2-butanyl]amino}-3-nitrophenyl)sulfonyl]benzamide and navitoclax (formerly ABT-263).

Exemplary gamma secretase inhibitors include compounds of formula (I) or a pharmaceutically acceptable salt thereof;

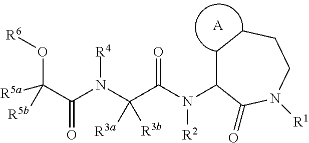

where ring A is aryl or heteroaryl; each of R¹, R², and R⁴ is independently hydrogen, C₁-C₆ alkyl, cycloalkyl, heterocyclyl, cycloalkylalkyl, heterocyclylalkyl, aryl, heteroaryl, aralkyl, or heteroaralkyl, where each C₁-C₆ alkyl, cycloalkyl, heterocyclyl, cycloalkylalkyl, heterocyclylalkyl, aryl, heteroaryl, aralkyl, or heteroaralkyl is substituted with 0-6 independent occurrences of halogen, —OR^{A}, —SR^{A}, —C(O)OR^{A}, —C(O)N(R^{A})(R^{B}), —N(R^{A})(R^{B}), or —C(NR^{C})N(R^{A})(R^{B}); each R^{3a}, R^{3b}, R^{5a}, and R^{5b} is independently hydrogen, halogen, —OH, C₁-C₆ alkyl, C₁-C₆ alkoxy, cycloalkyl, heterocyclyl, cycloalkylalkyl, heterocyclylalkyl, aryl, heteroaryl, aralkyl, or heteroaralkyl, where each C₁-C₆ alkyl, C₁-C₆ alkoxy, cycloalkyl, heterocyclyl, cycloalkylalkyl, heterocyclylalkyl, aryl, heteroaryl, aralkyl, or heteroaralkyl is substituted with 0-6 independent occurrences of halogen, —OH, —OR^{A}, —SR^{A}, —C(O)OR^{A}, —C(O)N(R^{A})(R^{B}), —N(R^{A})(R^{B}), or —C(NR^{C})N(R^{A})(R^{B}); R⁶ is hydrogen, C₁-C₆ alkyl, cycloalkyl, heterocyclyl, cycloalkylalkyl, heterocyclylalkyl, aryl, heteroaryl, aralkyl, or heteroaralkyl, where each C₁-C₆ alkyl, cycloalkyl, heterocyclyl, cycloalkylalkyl, heterocyclylalkyl, aryl, heteroaryl, aralkyl, or heteroaralkyl is substituted with 0-6 independent occurrences of halogen, —OH, or C₁-C₆ alkoxy; and each R^{A}, R^{B}, and RC is independently hydrogen, C₁-C₆ alkyl, cycloalkyl, heterocyclyl, cycloalkylalkyl, heterocyclylalkyl, aryl, heteroaryl, aralkyl, or heteroaralkyl, where each C₁-C₆ alkyl, cycloalkyl, heterocyclyl, cycloalkylalkyl, heterocyclylalkyl, aryl, heteroaryl, aralkyl, or heteroaralkyl is substituted with 0-6 independent occurrences of halogen, —OH, or C₁-C₆ alkoxy.

In an embodiment, the compound of formula (I) is a compound described in U.S. Pat. No. 7,468,365. In yet another embodiment, the compound is

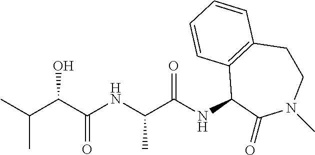 [SMILES: CC(C)[C@H](O)C(=O)N[C@@H](C)C(=O)N[C@@H]1C(=O)N(C)CCc2ccccc21]

or a pharmaceutically acceptable salt thereof.

The GSI can be a compound of formula (II) or a pharmaceutically acceptable salt thereof;

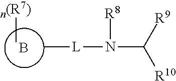

where ring B is aryl or heteroaryl; L is a bond, C₁-C₆ alkylene, —S(O)₂—, —C(O)—, —N(R^{E})(O)C—, or —OC(O)—; each R⁷ is independently halogen, —OH, C₁-C₆ alkyl, C₁-C₆ alkoxy, cycloalkyl, heterocyclyl, cycloalkylalkyl, heterocyclylalkyl, aryl, heteroaryl, aralkyl, or heteroaralkyl, where each C₁-C₆ alkyl, C₁-C₆ alkoxy, cycloalkyl, heterocyclyl, cycloalkylalkyl, heterocyclylalkyl, aryl, heteroaryl, aralkyl, or heteroaralkyl is independently substituted with 0-6 occurrences of halogen, —OR^{D}, —SR^{D}, —C(O)OR^{D}, —C(O)N(R^{D})(R^{E}), —N(R^{D})(R^{E}), or —C(NR^{F})N(R^{D})(R^{E}); R⁸ is hydrogen, C₁-C₆ alkyl, C₁-C₆ alkoxy, cycloalkyl, heterocyclyl, cycloalkylalkyl, heterocyclylalkyl, aryl, heteroaryl, aralkyl, or heteroaralkyl, where each C₁-C₆ alkyl, C₁-C₆ alkoxy, cycloalkyl, heterocyclyl, cycloalkylalkyl, heterocyclylalkyl, aryl, heteroaryl, aralkyl, or heteroaralkyl is substituted with 0-6 independent occurrences of halogen, —OR^{D}, —SR^{D}, —C(O)OR^{D}, —C(O)N(R^{D})(R^{E}), —N(R^{D})(R^{E}), or —C(NR^{F})N(R^{D})(R^{E}); each of R⁹ and R¹⁰ is independently hydrogen, halogen, —OH, C₁-C₆ alkyl, C₁-C₆ alkoxy, cycloalkyl, heterocyclyl, cycloalkylalkyl, heterocyclylalkyl, aryl, heteroaryl, aralkyl, or heteroaralkyl, where each C₁-C₆ alkyl, C₁-C₆ alkoxy, cycloalkyl, heterocyclyl, aryl, heteroaryl, aralkyl, or heteroaralkyl is substituted with 0-6 independent occurrences of halogen, —OR^{D}, —SR^{D}, —C(O)OR^{D}, —C(O)N(R^{D})(R^{E}), —N(R^{D})(R^{E}), or —C(NR^{I})N(R^{G})(R^{H}); each R^{D}, R^{E}, and R^{F} is independently hydrogen, C₁-C₆ alkyl, cycloalkyl, heterocyclyl, cycloalkylalkyl, heterocyclylalkyl, aryl, heteroaryl, aralkyl, or heteroaralkyl, where each C C₁-C₆ alkyl, cycloalkyl, heterocyclyl, cycloalkylalkyl, heterocyclylalkyl, aryl, heteroaryl, aralkyl, or heteroaralkyl is substituted with 0-6 independent occurrences of halogen, —OH, or C₁-C₆ alkoxy; and n is 0, 1, 2, 3, 4, or 5.

In a further embodiment, the compound of formula (II) is a compound described in U.S. Pat. No. 7,687,666. In yet another embodiment, the compound is

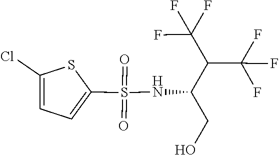 [SMILES: O=S(=O)(N[C@@H](CO)C(C(F)(F)F)C(F)(F)F)c1ccc(Cl)s1]

or a pharmaceutically acceptable salt thereof.

In some embodiments, the GSI is a compound is a compound of formula (III) or a pharmaceutically acceptable salt thereof:

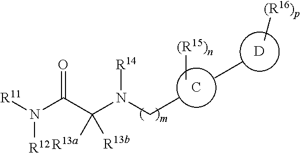

where each of rings C and D is independently aryl or heteroaryl;
each of R¹¹, R¹², and R¹⁴ is independently hydrogen, C₁-C₆ alkyl, C₁-C₆ alkoxy, cycloalkyl, heterocyclyl, cycloalkylalkyl, heterocyclylalkyl, aryl, heteroaryl, aralkyl, or heteroaralkyl, where each C₁-C₆ alkyl, C₁-C₆ alkoxy, cycloalkyl, heterocyclyl, cycloalkylalkyl, heterocyclylalkyl, aryl, heteroaryl, aralkyl, or heteroaralkyl is substituted with 0-6 independent occurrences of halogen, —OR^{G}, —SR^{G}, —C(O)OR^{G}, —C(O)N(R^{G})(R^{H}), —N(R^{G})(R^{H}), or —C(NR^{I})N(R^{G})(R^{H}); each of R^{13a} and R^{13b} is hydrogen, halogen, —OH, C₁-C₆ alkyl, C₁-C₆ alkoxy, cycloalkyl, heterocyclyl, cycloalkylalkyl, heterocyclylalkyl, aryl, heteroaryl, aralkyl, or heteroaralkyl, where each C₁-C₆ alkyl, C₁-C₆ alkoxy, cycloalkyl, heterocyclyl, cycloalkylalkyl, heterocyclylalkyl, aryl, heteroaryl, aralkyl, or heteroaralkyl is substituted with 0-6 independent occurrences of halogen, —OR^{G}, —SR^{G}, —C(O)OR^{G}, —C(O)N(R^{G})(R^{H}), —N(R^{G})(R^{H}), or —C(NR^{I})N(R^{G})(R^{H}); each R¹⁵ and R¹⁶ is independently halogen, —OH, C₁-C₆ alkyl, C₁-C₆ alkoxy, cycloalkyl, heterocyclyl, cycloalkylalkyl, heterocyclylalkyl, aryl, heteroaryl, aralkyl, or heteroaralkyl, where each C₁-C₆ alkyl, C₁-C₆ alkoxy, cycloalkyl, heterocyclyl, cycloalkylalkyl, heterocyclylalkyl, aryl, heteroaryl, aralkyl, or heteroaralkyl is substituted with 0-6 independent occurrences of halogen, —OR^{G}, SR^{G}, —C(O)OR^{G}, —C(O)N(R^{G})(R^{H}), —N(R^{G})(R^{H}), or —C(NR^{I})N(R^{G})(R^{H}); each R^{G}, R^{H}, and R^{I} is independently hydrogen, C₁-C₆ alkyl, cycloalkyl, heterocyclyl, cycloalkylalkyl, heterocyclylalkyl, aryl, heteroaryl, aralkyl, or heteroaralkyl, where each C₁-C₆ alkyl, cycloalkyl, heterocyclyl, cycloalkylalkyl, heterocyclylalkyl, aryl, heteroaryl, aralkyl, or heteroaralkyl is substituted with 0-6 independent occurrences of halogen, —OH, or C₁-C₆ alkoxy; and each of m, n, and p is independently 0, 1, 2, 3, 4, or 5.

In a further embodiment, the GSI is a compound described in U.S. Pat. No. 8,084,477. In yet another embodiment, the GSI is

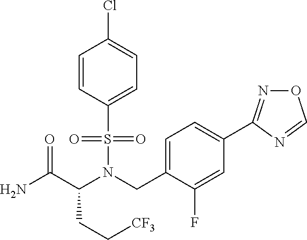 [SMILES: NC(=O)[C@@H](CCC(F)(F)F)N(Cc1ccc(-c2ncon2)cc1F)S(=O)(=O)c1ccc(Cl)cc1]

or a pharmaceutically acceptable salt thereof.

In some embodiments, the GSI is a compound described in U.S. Pat. No. 7,160,875. In some embodiments, the gamma secretase inhibitor is a compound of formula (IV) or a pharmaceutically acceptable salt thereof:

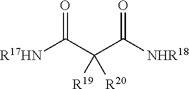

where R¹⁷ is selected from

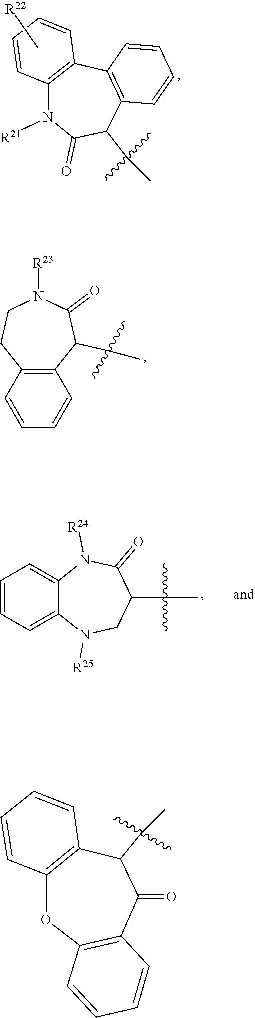

R¹⁸ is lower alkyl, lower alkinyl, —(CH₂)ₙ—O-lower alkyl, —(CH₂)ₙ—S-lower alkyl, —(CH₂)ₙ—CN, —(CR′R″)ₙ—CF₃, —(CR′R″)ₙ—CHF₂, —(CR′R″)ₙ—CH₂F, —(CH₂)ₙ, —C(O)O-lower alkyl, —(CH₂)ₙ-halogen, or is —(CH₂)ₙ—cycloalkyl optionally substituted by one or more substituents selected from the group consisting of phenyl, halogen and CF₃; R′,R″ are each independently hydrogen, lower alkyl, lower alkoxy, halogen or hydroxy; R¹⁹, R²⁰ are each independently hydrogen, lower alkyl, lower alkoxy, phenyl or halogen; R²¹ is hydrogen, lower alkyl, —(CH₂)ₙ—CF₃ or —(CH₂)ₙ-cycloalkyl; R²² is hydrogen or halogen; R²³ is hydrogen or lower alkyl; R²⁴ is hydrogen, lower alkyl, lower alkinyl, —(CH₂)ₙ—CF₃, —(CH₂)ₙ-cycloalkyl or —(CH₂)ₙ-phenyl optionally substituted by halogen; R²⁵ is hydrogen, lower alkyl, —C(O)H, —C(O)-lower alkyl, —C(O)—CF₃, —C(O)—CH₂F, —C(O)—CHF₂, —C(O)— cycloalkyl, —C(O)—(CH₂)ₙ—O-lower alkyl, —C(O)O—(CH₂)ₙ-cycloalkyl, —C(O)-phenyl optionally substituted by one or more substituents selected from the group consisting of halogen and —C(O)O-lower alkyl, or is —S(O)₂-lower alkyl, —S(O)₂—CF₃, —(CH₂)ₙ-cycloalkyl or is —(CH₂)ₙ-phenyl optionally substituted by halogen; n is 0, 1, 2, 3 or 4.

In some embodiments, the GSI is

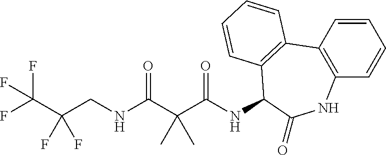 [SMILES: CC(C)(C(=O)NCC(F)(F)C(F)(F)F)C(=O)N[C@@H]1C(=O)Nc2ccccc2-c2ccccc21]

or a pharmaceutically acceptable salt thereof.

In some embodiments, the GSI is a compound described in U.S. Pat. No. 6,984,663. In some embodiments, the GSI is a compound of Formula (V) or a pharmaceutically acceptable salt thereof:

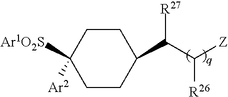

where
q is 0 or 1; Z represents halogen, —CN, —NO₂, —N₃, —CF₃, —OR^{2a}, —N(R^{2a})₂, —CO₂R^{2a}, —OCOR^{2a}, —COR^{2a}, —CON(R^{2a})₂, —OCON(R^{2a})₂, —CONR^{2a}(OR^{2a}), —CON(R^{2a})N(R^{2a})₂, —CONHC(═NOH)R^{2a}, heterocyclyl, phenyl or heteroaryl, the heterocyclyl, phenyl or heteroaryl bearing 0-3 substituents selected from halogen, —CN, —NO₂, —CF₃, —OR^{2a}, —N(R^{2a})₂, —CO₂R^{2a}, —COR^{2a}, —CON(R^{2a})₂ and C₁₋₄ alkyl; R²⁷ represents H, C₁₋₄ alkyl, or OH; R²⁶ represents H or C₁₋₄ alkyl; with the proviso that when m is 1, R²⁶ and R²⁷ do not both represent C₁₋₄ alkyl; Ar¹ represents C₆₋₁₀ aryl or heteroaryl, either of which bears 0-3 substituents independently selected from halogen, —CN, —NO₂, —CF₃, —OH, —OCF₃, C₁₋₄alkoxy or C₁₋₄alkyl which optionally bears a substituent selected from halogen, CN, NO₂, CF₃, OH and C₁₋₄alkoxy; Ar² represents C₆₋₁₀ aryl or heteroaryl, either of which bears 0-3 substituents independently selected from halogen, —CN, —NO₂, —CF₃, —OH, —OCF₃, C₁₋₄alkoxy or C₁₋₄alkyl which optionally bears a substituent selected from halogen, —CN, —NO₂, —CF₃, —OH and C₁₋₄alkoxy; R^{2a} represents H, C₁₋₆ alkyl, C₃₋₆ cycloalkyl, C3_6cycloalkyl, C₁₋₆ alkyl, C₂₋₆ alkenyl, any of which optionally bears a substituent selected from halogen, —CN, —NO₂, —CF₃, —OR^{2b}, —CO₂R^{2b}, —N(R^{2b})₂, —CON(R^{2b})₂, Ar and COAr; or R^{2a} represents Ar; or two R^{2a} groups together with a nitrogen atom to which they are mutually attached may complete an N-heterocyclyl group bearing 0-4 substituents independently selected from ═O, ═S, halogen, C₁₋₄alkyl, —CN, —NO₂, —CF₃, —OH, C₁₋₄alkoxy, C₁₋₄ alkoxycarbonyl, CO₂H, amino, C₁₋₄alkylamino, di(C₁₋₄alkyl)amino, carbamoyl, Ar and COAr; R^{2b} represents H, C₁₋₆ alkyl, C₃₋₆ cycloalkyl, C₃₋₆ cycloalkylC₁₋₆ alkyl, C₂₋₆ alkenyl, any of which optionally bears a substituent selected from halogen, —CN, —NO₂, —CF₃, —OH, C₁₋₄alkoxy, C₁₋₄ alkoxycarbonyl, —CO₂H, amino, C₁₋₄alkylamino, di(C₁₋₄alkyl)amino, carbamoyl, Ar and COAr; or R^{2b} represents Ar; or two R^{2b} groups together with a nitrogen atom to which they are mutually attached may complete an N-heterocyclyl group bearing 0-4 substituents independently selected from ═O, ═S, halogen, C₁₋₄alkyl, —CN, —NO₂, CF3, —OH, C₁₋₄alkoxy, C₁₋₄ alkoxycarbonyl, —CO₂H, amino, C₁₋₄alkylamino, di(C₁₋₄alkyl)amino, carbamoyl, Ar and COAr; Ar represents phenyl or heteroaryl bearing 0-3 substituents selected from halogen, C₁₋₄alkyl, —CN, —NO₂, —CF₃, —OH, C₁₋₄ alkoxy, C₁₋₄ alkoxycarbonyl, amino, C₁₋₄ alkylamino, di(C₁₋₄alkyl)amino, carbamoyl, C₁₋₄ alkylcarbamoyl and di(C₁₋₄ alkyl)carbamoyl.

In some embodiments, the GSI is

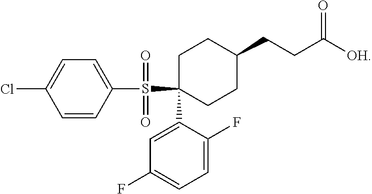 [SMILES: O=C(O)CC[C@H]1CC[C@@](c2cc(F)ccc2F)(S(=O)(=O)c2ccc(Cl)cc2)CC1]

or a pharmaceutically acceptable salt thereof.

In some embodiments, the GSI is a compound described in U.S. Pat. No. 7,795,447. In some embodiments, the GSI is a compound of formula (VI) or a pharmaceutically acceptable salt thereof.

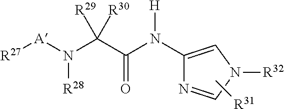

where A′ is absent or selected from

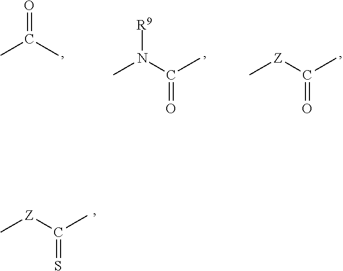

### and —S(O)₂—;

Z is selected from —CH₂, —CH(OH), —CH(C₁-C₆ alkyl), —CH(C₁-C₆ alkoxy), —CH(NR³³R³⁴), —CH(CH₂(OH)), —CH(CH(C₁-C₄ alkyl)(OH)) and —CH(C(C₁-C₄ alkyl)(C₁-C₄ alkyl)(OH)), for example —CH(C(CH₃)(CH₃)(OH)) or —CH(C(CH₃)(CH₂CH₃)(OH)); R²⁷ is selected from C₁-C₂₀ alkyl, C₂-C₂₀ alkenyl, C₂-C₂₀ alkynyl, C₁-C₂₀ alkoxy, C₂-C₂₀ alkenoxy, C₁-C₂₀ hydroxyalkyl, C₃-C₈ cycloalkyl, benzo(C₃-C₈ cycloalkyl), benzo(C₃-C₈ heterocycloalkyl), C₄-C₈ cycloalkenyl, (C₅-C₁₁)bi- or tricycloalkyl, benzo(C₅-C₁₁)bi- or tricycloalkyl, C₇-C₁₁tricycloalkenyl, (3-8 membered) heterocycloalkyl, C₆-C₁₄ aryl and (5-14 membered) heteroaryl, where each hydrogen atom of the alkyl, alkenyl, alkynyl, alkoxy and alkenoxy is optionally independently replaced with halo, and where the cycloalkyl, benzo(C₃-C₈ cycloalkyl), cycloalkenyl, (3-8 membered) heterocycloalkyl, C₆-C₁₄ aryl and (5-14 membered) heteroaryl is optionally independently substituted with from one to four substituents independently selected from C₁-C₁₀ alkyl optionally substituted with from one to three halo atoms, C₁-C₁₀ alkoxy optionally substituted with from one to three halo atoms, C₁-C₁₀ hydroxyalkyl, halo, e.g., fluorine, —OH, —CN, —NR³³R³⁴, —C(═O)NR³³R³⁴, —C(═O)R³⁵, C₃-C₈ cycloalkyl and (3-8 membered) heterocycloalkyl; R²⁸ is selected from H, C₁-C₆ alkyl, C₂-C₆ alkenyl, C₃-C₈ cycloalkyl and C₅-C₈ cycloalkenyl, where R²⁸ is optionally independently substituted with from one to three substituents independently selected from C₁-C₄ alkyl optionally substituted with from one to three halo atoms, C₁-C₄ alkoxy optionally substituted with from one to three halo atoms, halo and —OH; or R²⁷ and R²⁸ together with the A′ group when present and the nitrogen atom to which R² is attached, or R¹ and R² together with the nitrogen atom to which R²⁷ and R²⁸ are attached when A is absent, may optionally form a four to eight membered ring; R²⁹ is selected from H, C₁-C₆ alkyl, C₂-C₆ alkenyl, C₂-C₆ alkynyl, C₃-C₆ cycloalkyl, C₅-C₆ cycloalkenyl and (3-8 membered) heterocycloalkyl, where the alkyl, alkenyl, alkynyl, cycloalkyl, cycloalkenyl and heterocycloalkyl are each optionally independently substituted with from one to three substituents independently selected from C₁-C₄alkoxy, halo, —OH—S(C₁-C₄)alkyl and (3-8 membered) heterocycloalkyl; R³⁰ is H, C₁-C₆ alkyl or halo; or R³ and R⁴ may together with the carbon atom to which they are attached optionally form a moiety selected from cyclopropyl, cyclobutyl, cyclopentyl, cyclohexyl, morpholino, piperidino, pyrrolidino, tetrahydrofuranyl and perhydro-2H-pyran, where the moiety formed by R²⁹ and R³⁰ is optionally substituted with from one to three substituents independently selected from C₁-C₆ alkyl optionally substituted with from one to three halo atoms, C₁-C₆ alkoxy optionally substituted with from one to three halo atoms, halo, —OH, —CN and allyl; R³¹ is selected from H, C₁-C₆ alkyl, C₂-C₆ alkylene, C₁-C₆ alkoxy, halo, —CN, C₃-C₁₂ cycloalkyl, C₄-C₁₂ cycloalkenyl and C₆-C₁₀ aryl, (5-10 membered) heteroaryl, where the alkyl, alkylene and alkoxy of R³¹ are each optionally independently substituted with from one to three substituents independently selected from halo and —CN, and where the cycloalkyl, cycloalkenyl and aryl and heteroaryl of R³¹ are each optionally independently substituted with from one to three substituents independently selected from C₁-C₄ alkyl optionally substituted with from one to three halo atoms, C₁-C₄ alkoxy optionally substituted with from one to three halo atoms, halo and —CN; R³² is selected from H, C₁-C₂₀ alkyl, C₁-C₂₀ alkoxy, C₁-C₂₀ hydroxyalkyl, C₃-C₁₂ cycloalkyl, C₄-C₁₂ cycloalkenyl, (C₅-C₂₀) bi- or tricycloalkyl, (C₇-C₂₀)bi- or tricycloalkenyl, (3-12 membered) heterocycloalkyl, (7-20 membered) hetero bi- or heterotricycloalkyl, C₆-C₁₄ aryl and (5-15 membered) heteroaryl, where R³² is optionally independently substituted with from one to four substituents independently selected from C₁-C₂₀ alkyl optionally substituted with from one to three halo atoms, C₁-C₂₀ alkoxy, —OH, —CN, —NO₂, —NR³³R³⁴, —C(═O)NR³³R³⁴, —C(═O)R³⁵, —C(═O)OR³⁵, —S(O)ₙNR³³R³⁴, —S(O)ₙR³⁵, C₃-C₁₂ cycloalkyl, (4-12 membered) heterocycloalkyl optionally substituted with from one to three OH or halo groups, (4-12 membered) heterocycloalkoxy, C₆-C₁₄ aryl, (5-15 membered) heteroaryl, C₆-C₁₂ aryloxy and (5-12 membered) heteroaryloxy; or R⁶ and R⁷ may together with the carbon and nitrogen atoms to which they are respectively attached optionally form a (5-8 membered) heterocycloalkyl ring, a (5-8 membered) heterocycloalkenyl ring or a (6-10 membered) heteroaryl ring, where the heterocycloalkyl, heterocycloalkenyl and heteroaryl rings are each optionally independently substituted with from one to three substituents independently selected from halo, C₁-C₆ alkyl, optionally substituted with from one to three halo atoms, C₁-C₆ alkoxy optionally substituted with from one to three halo atoms, C₁-C₆ hydroxyalkyl, —OH, —(CH₂)_{zero-10}NR³³R³⁴, —(CH₂)_{zero-10}C(═O)NR³³R³⁴, —S(O)₂NR³³R³⁴ and C₃-C₁₂ cycloalkyl; R³³ and R³⁴ are each independently selected from H, C₁-C₁₀ alkyl where each hydrogen atom of the C₁-C₁₀ alkyl is optionally independently replaced with a halo atom, e.g., a fluorine atom, C₂-C₁₀ alkenyl, C₂-C₁₀ alkynyl, C₁-C₆ alkoxy where each hydrogen atom of the C₁-C₆ alkoxy is optionally independently replaced with a halo atom, C₂-C₆ alkenoxy, C2-C6 alkynoxy, —C(═O)R11, —S(O)nR11, C₃-C₈ cycloalkyl, C₄-C₈ cycloalkenyl, (C₅-C₁₁)bi- or tricycloalkyl, (C₇-C₁₁)bi- or tricycloalkenyl, (3-8 membered) heterocycloalkyl, C₆-C₁₄ aryl and (5-14 membered) heteroaryl, where the alkyl and alkoxy are each optionally independently substituted with from one to three substituents independently selected from halo and —OH, and where the cycloalkyl, cycloalkenyl, bi- or tricycloalkyl, bi- or tricycloalkenyl, heterocycloalkyl, aryl and heteroaryl are each optionally independently substituted with from one to three substituents independently selected from halo, —OH, C₁-C₆ alkyl optionally independently substituted with from one to six halo atoms, C₂-C₆ alkenyl, C₂-C₆ alkynyl, C₁-C₆ alkoxy, C₂-C₆ alkenoxy, C₂-C₆ alkynoxy and C₁-C₆ hydroxyalkyl; or NR³³R³⁴ may form a (4-7 membered) heterocycloalkyl, where the heterocycloalkyl optionally comprises from one to two further heteroatoms independently selected from N, O, and S, and where the heterocycloalkyl optionally contains from one to three double bonds, and where the heterocycloalkyl is optionally independently substituted with from one to three substituents independently selected from C₁-C₆ alkyl optionally substituted with from one to six halo atoms, C₂-C₆ alkenyl, C₂-C₆ alkynyl, C₁-C₆ alkoxy, C₂-C₆ alkenoxy, C₂-C₆ alkynoxy, C₁-C₆ hydroxyalkyl, C₂-C₆hydroxyalkenyl, C₂-C₆hydroxyalkynyl, halo, —OH, —CN, —NO₂, —C(═O)R³⁵, —C(═O)OR³⁵, —S(O)ₙR³⁵ and —S(O)ₙNR³³R³⁴; R³⁵ is selected from H, C₁-C₈ alkyl, C₃-C₈ cycloalkyl, C₄-C₈ cycloalkenyl, (C₅-C₁₁)bi- or tricycloalkyl, —(C₇-C₁)bi- or tricycloalkenyl, (3-8 membered) heterocycloalkyl, C₆-C₁₀ aryl and (5-14 membered) heteroaryl, where the alkyl of R³⁵ is optionally independently substituted with from one to three substituents independently selected from —OH, —CN and C₃-C₈ cycloalkyl, and where each hydrogen atom of the alkyl is optionally independently replaced with a halo atom, e.g., a fluorine atom, and where the cycloalkyl, cycloalkenyl, heterocycloalkyl, aryl and hetereoaryl of R³⁵ are each optionally independently substituted with from one to three substituents independently selected from halo, C₁-C₈ alkyl optionally substituted with from one to three halo atoms, —OH, —CN and C₃-C₈cycloalkyl; n is in each instance an integer independently selected from zero, 1, 2 and 3; and the pharmaceutically acceptable salts of such compounds.

In some embodiments, the GSI is

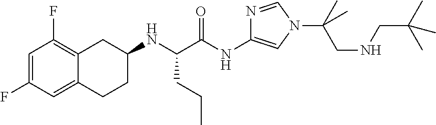 [SMILES: CCC[C@H](N[C@H]1CCc2cc(F)cc(F)c2C1)C(=O)Nc1cn(C(C)(C)CNCC(C)(C)C)cn1]

or a pharmaceutically acceptable salt thereof.

In some embodiments, the GSI is an antibody molecule that reduces the expression and/or function of gamma secretase. In some embodiments, the GSI is an antibody molecule targeting a subunit of gamma secretase. In some embodiments, the GSI is chosen from an anti-presenilin antibody molecule, an anti-nicastrin antibody molecule, an anti-APH-1 antibody molecule, or an anti-PEN-2 antibody molecule.

Exemplary antibody molecules that target a subunit of gamma secretase (e.g., e.g., presenilin, nicastrin, APH-1, or PEN-2) are described in U.S. Pat. Nos. 8,394,376, 8,637,274, and 5,942,400.

Gamma secretase modulators described in WO 2017/019496 can also be used. In some embodiments, the gamma secretase modulator is γ-secretase inhibitor I (GSI I) Z-Leu-Leu-Norleucine; γ-secretase inhibitor II (GSI II); γ-secretase inhibitor III (GSI III), N-Benzyloxycarbonyl-Leu-leucinal, N-(2-Naphthoyl)-Val-phenylalaninal; γ-secretase inhibitor IV (GSI IV); γ-secretase inhibitor V (GSI V), N-Benzyloxycarbonyl-Leu-phenylalaninal; γ-secretase inhibitor VI (GSI VI), 1-(S)-endo-N-(1,3,3)-Trimethylbicyclo[2.2.1]hept-2-yl)-4-fluorophenyl Sulfonamide; γ-secretase inhibitor VII (GSI VII), Menthyloxycarbonyl-LL-CHO; γ-secretase inhibitor IX (GSI IX), (DAPT), N—[N-(3,5-Difluorophenacetyl-L-alanyl)]-S-phenylglycine t-Butyl Ester; γ-secretase inhibitor X (GSI X), {1 S-Benzyl-4R-[1-(1S-carbamoyl-2-phenethylcarbamoyl)-1S-3-methylbutylcarb-amoyl]-2R-hydroxy-5-phenylpentyl}carbamic Acid tert-butyl Ester; γ-secretase inhibitor XI (GSI XI), 7-Amino-4-chloro-3-methoxyisocoumarin; γ-secretase inhibitor XII (GSI XII), Z-Ile-Leu-CHO; γ-secretase inhibitor XIII (GSI XIII), Z-Tyr-Ile-Leu-CHO; γ-secretase inhibitor XIV (GSI XIV), Z-Cys(t-Bu)-Ile-Leu-CHO; γ-secretase inhibitor XVI (GSI XVI), N—[N-3,5-Difluorophenacetyl]-L-alanyl-S-phenylglycine Methyl Ester; γ-secretase inhibitor XVII (GSI XVII); γ-secretase inhibitor XIX (GSI XIX), benzo[e][I,4]diazepin-3-yl)-butyramide; γ-secretase inhibitor XX (GSI XX), (S,S)-2-[2-(3,5-Difluorophenyl)acetylamino]-N-(5-methyl-6-oxo-6,7-dihydro-5H-dibenzo[b,d]azepin-7-yl)propionamide; γ-secretase inhibitor XXI (GSI XXI), (S,S)-2-[2-(3,5-Difluorophenyl)-acetylamino]-N—(I-methyl-2-oxo-5-phenyl-2-,3-dihydro-IH-benzo[e][1,4]diazepin-3-yl)-propionamide; Gamma40 secretase inhibitor I, N-trans-3,5-Dimethoxycinnamoyl-Ile-leucinal; Gamma40 secretase inhibitor II, N-tert-Butyloxycarbonyl-Gly-Val-Valinal Isovaleryl-V V-Sta-A-Sta-OCH3; MK-0752 (Merck); MRK-003 (Merck); semagacestat/LY450139 (Eli Lilly); RO4929097; PF-03084014; BMS-708163; MPC-7869 (γ-secretase modifier), YO-01027 (Dibenzazepine); LY411575 (Eli Lilly and Co.); L-685458 (Sigma-Aldrich); BMS-289948 (4-chloro-N-(2,5-difluorophenyl)-N—((IR)-{4-fluoro-2-[3-(IH-imidazol-1-yl)propyl]phenyl}ethyl)benzenesulfonamide hydrochloride); or BMS-299897 (4-[2-((IR)—I-{[(4-chlorophenyl)sulfonyl]-2,5-difluoroanilino}ethyl)-5-fluorophenyljbutanoic acid) (Bristol Myers Squibb).

Exemplary cytokine therapies include interleukin 2 (IL-2) and interferon-alpha (IFN-alpha).

In certain aspects, “cocktails” of different chemotherapeutic agents are administered as the additional agent(s).

In some embodiments, the additional agent(s) to be administered in combination with the MBMs of the disclosure are one or more standard of care agents or therapies and/or experimental treatments.

For Hodgkin's lymphoma, combination agents/therapies include radiation and/or chemotherapy (e.g., ABVD (doxorubicin, bleomycin, vinblastine, and dacarbazine), BEACOPP (bleomycin, etoposide, doxorubicin, cyclophosphamide, vincristine, procarbazine and prednisone), or Stanford V (doxorubicin, mechlorethamine (nitrogen mustard), vincristine, vinblastine, bleomycin, etoposide, and prednisone)), antibodies (e.g., brentuximab vedotin, rituximab, or a checkpoint inhibitor such as nivolumab or pembrolizumab).

For DLBCL, combination agents/therapies include monoclonal antibodies (e.g., rituximab (Rituxan)), chemotherapy and/or radiation.

For follicular lymphoma, combination agents/therapies include chemotherapy (e.g., bendamustine (Treanda)); monoclonal antibodies (e.g., rituximab), targeted therapies (e.g., lenalidomide (Revlimid)) and/or radiation.

For mantle cell lymphoma, combination agents/therapies include chemotherapy (including high dose chemotherapy), monoclonal antibodies (e.g., rituximab), targeted therapies (e.g., bortezomib (Velcade), ibrutinib (Imbruvica), and lenalidomide (Revlimid)), stem cell transplants and/or radiation therapy.

For small lymphocytic lymphoma, combination agents/therapies include chemotherapy, monoclonal antibodies, stem cell transplantation, targeted therapies (e.g., ibrutinib), and/or tumor vaccines.

For primary mediastinal large B-cell lymphoma and mediastinal grey-zone lymphoma (MGZL), combination agents/therapies include anthracycline-based chemotherapy, rituximab and/or radiation therapy to the chest.

For splenic marginal zone B-cell lymphoma, combination agents/therapies include the same treatments as follicular lymphoma and additionally in some cases removal of the spleen.

For extranodal marginal zone B-cell lymphoma of MALT, combination agents/therapies include antibiotics (to treat the often causal infection with Helicobacter pylori), radiation therapy, surgery, chemotherapy, and/or monoclonal antibodies.

For nodal marginal zone B-cell lymphoma, combination agents/therapies include the same treatments as follicular lymphoma.

For lymphoplasmacytic lymphoma and the Waldenstrom's macroglobulinemia (WM) variant, combination agents/therapies include those useful for chronic lymphocytic leukemia or follicular lymphoma (see above).

For primary effusion lymphoma, combination agents/therapies include those useful for other diffuse large-cell lymphomas.

For Burkitt lymphoma/Burkitt cell leukemia, combination agents/therapies include intensive chemotherapy.

For multiple myeloma, combination agents/therapies include dexamethasone, pomalidomide (with or without dexamethasone), lenalidomide (with or without dexamethasone), and bortezomib (with or without dexamethasone).

### 8. EXAMPLES

### 8.1. Example 1: Production and Characterization of Anti-CD19-Anti-CD20-Anti-CD3 Human IgG1 Bi- and Tri-Specific Binding Molecules

### 8.1.1. Materials and Methods

### 8.1.1.1. Gene Construction, Expression and Purification of Bi- and Tri-Specific Binding Molecules

Gene synthesis for all constructs were synthesized externally and codon optimized for expression in mammalian cells. For anti-CD3, anti-CD19 and anti-CD3 arms which contained single specificity, these were synthesized by encoding nucleotide sequences for variable heavy chain region followed by full Fc sequence similar to a traditional antibody. Corresponding full light chain plasmids were also synthesized. For the multi-specific construct containing both anti-CD19 and anti-CD3 specificities, this was synthesized by encoding VL of CD19 fused to CL followed by a linker followed by the full heavy chain sequence and Fc of CD3 antibody. Plasmids encoding the light chain of the anti-CD3 arm and the heavy chain variable sequence and CH₁ sequence for the anti-CD19 arm were also synthesized. Constant human IgG1 sequence contained additional mutations which silence antibody dependent cellular cytotoxicity and mutations which facilitate production of bispecific antibodies after expression. Amino acid sequences encoded by the constructs are shown in Table 13.

Anti-CD19, anti-CD20 and anti-CD3 antibodies were expressed transiently in Human Embryonic Kidney (HEK293) cells. Briefly, transfection was performed using PEI Max (Polyethylenimine, MW 40.000 linear, Polysciences, USA Cat. No. 24765-2) as transfection reagent. For small scale (<5 L) transfections, cells are grown in shake flasks on an orbital shaker (115 rpm) in a humidified incubator (85%) at 5% CO2). Light and heavy chain plasmids were combined with PEI at a final ratio of 1 DNA:3 PEI. 1 mg/L culture of plasmid was used for transfection at 0.5 million cells/mL serum media. After 7 days of expression, the antibody was harvested by clarification of the media via centrifugation and filtration. Purification was performed via Protein A affinity chromatography (HiTrap—MabSelect®SuRe, GE Healthcare Life Sciences, Uppsala, Sweden) on an FPLC. The column was loaded with supernatant, washed with 13 CV of PBS. Antibody was eluted with 5 CV of 50 mM citrate, 90 mM NaCl pH 3.2. The eluted IgG protein was adjusted to pH7 with 1 M Tris HCl pH10. If the antibody contained aggregates, preparative size exclusion chromatography was performed using Hi Load 16/60 Superdex 200 grade column (GE Healthcare Life Sciences, Uppsala, Sweden) as a final polishing step.

Using purified antibodies, bispecific and trispecific antibodies were produced as described in Labrijin et. al., 2014. Antibodies containing the desired combination of targeting arms were mixed at 1:1 ratio and incubated in the presence of 2-mercaptoethylamine to reduce the inter-heavy chain disulfide bonds. The protein was buffer exchanged to remove the 2-MEA reductant and the protein disulfide bonds allowed to re-oxidize to form the stable heterodimer. If the bi or trispecific antibody contained aggregates, preparative size exclusion chromatography was performed using Hi Load 16/60 Superdex 200 grade column (GE Healthcare Life Sciences, Uppsala, Sweden) as a final polishing step.

To confirm that the identity of the proteins expressed matched the predicted masses for the primary amino acid sequences shown in in Table 13, bispecific and multispecific proteins were analyzed by high-performance liquid chromatography coupled to mass spectrometry.

TABLE 13
Amino acid sequences for binding molecules of Example 1

| Description | SEQ ID No: | Amino acid sequence |
|---|---|---|
| CD19-CD3 | | |
| bispecific | | |
| CD19 light | 699 | EIVMTQSPATLSLSPGERATLSCRASQDISKYLNWYQQKPGQA |
| chain | | PRLLIYHTSRLHSGIPARFSGSGSGTDYTLTISSLQPEDFAVYFC |
| | | QQGNTLPYTFGQGTKLEIKRTVAAPSVFIFPPSDEQLKSGTASV |
| | | VCLLNNFYPREAKVQWKVDNALQSGNSQESVTEQDSKDSTYS |
| | | LSSTLTLSKADYEKHKVYACEVTHQGLSSPVTKSFNRGEC |
| CD19 heavy | 700 | QVQLQESGPGLVKPSETLSLTCTVSGVSLPDYGVSWIRQPPG |
| chain | | KGLEWIGVIWGSETTYYQSSLKSRVTISKDNSKNQVSLKLSSVT |
| | | AADTAVYYCAKHYYYGGSYAMDYWGQGTLVTVSSASTKGPS |
| | | VFPLAPSSKSTSGGTAALGCLVKDYFPEPVTVSWNSGALTSGV |
| | | HTFPAVLQSSGLYSLSSVVTVPSSSLGTQTYICNVNHKPSNTKV |
| | | DKRVEPKSCDKTHTCPPCPAPELLGGPSVFLFPPKPKDTLMIS |
| | | RTPEVTCVVVDVSHEDPEVKFNWYVDGVEVHNAKTKPREEQY |
| | | ASTYRVVSVLTVLHQDWLNGKEYKCKVSNKALPAPIEKTISKAK |
| | | GQPREPQVYTLPPSREEMTKNQVSLTCLVKGFYPSDIAVEWE |
| | | SNGQPENNYKTTPPVLDSDGSFFLYSRLTVDKSRWQQGNVFS |
| | | CSVMHEALHNHYTQKSLSLSPGK |
| CD3 light chain | 701 | QAVVTQEPSLTVSPGGTVTLTCRSSTGAVTTSNYANWVQQKP |
| | | GQAPRGLIGGTNKRAPWTPARFSGSLLGDKAALTLSGAQPED |
| | | EAEYFCALWYSNLWVFGGGTKLTVLGQPKAAPSVTLFPPSSE |
| | | ELQANKATLVCLISDFYPGAVTVAWKADSSPVKAGVETTTPSK |
| | | QSNNKYAASSYLSLTPEQWKSHRSYSCQVTHEGSTVEKTVAP |
| | | TECS |
| CD3 heavy | 702 | EVQLVESGGGLVQPGGSLKLSCAASGFTFNTYAMNWVRQAS |
| chain | | GKGLEWVGRIRSKYNNYATYYADSVKDRFTISRDDSKSTLYLQ |
| | | MNSLKTEDTAVYYCVRHGNFGNSYVSWFAYWGQGTLVTVSS |
| | | ASTKGPSVFPLAPSSKSTSGGTAALGCLVKDYFPEPVTVSWNS |
| | | GALTSGVHTFPAVLQSSGLYSLSSVVTVPSSSLGTQTYICNVN |
| | | HKPSNTKVDKRVEPKSCDKTHTCPPCPAPELLGGPSVFLFPPK |
| | | PKDTLMISRTPEVTCVVVDVSHEDPEVKFNWYVDGVEVHNAK |
| | | TKPREEQYASTYRVVSVLTVLHQDWLNGKEYKCKVSNKALPA |
| | | PIEKTISKAKGQPREPQVYTLPPSREEMTKNQVSLTCLVKGFYP |
| | | SDIAVEWESNGQPENNYKTTPPVLDSDGSFLLYSKLTVDKSRW |
| | | QQGNVFSCSVMHEALHNHYTQKSLSLSPGK |
| CD20-CD3 | | |
| bispecific | | |
| CD20 light | 703 | QIVLSQSPAILSASPGEKVTMTCRASSSVSYIHWFQQKPGSSP |
| chain | | KPWIYATSNLASGVPVRFSGSGSGTSYSLTISRVEAEDAATYY |
| | | CQQWTSNPPTFGGGTKLEIKRTVAAPSVFIFPPSDEQLKSGTA |
| | | SVVCLLNNFYPREAKVQWKVDNALQSGNSQESVTEQDSKDST |
| | | YSLSSTLTLSKADYEKHKVYACEVTHQGLSSPVTKSFNRGEC |
| CD20 heavy | 704 | QVQLQQPGAELVKPGASVKMSCKASGYTFTSYNMHWVKQTP |
| chain | | GRGLEWIGAIYPGNGDTSYNQKFKGKATLTADKSSSTAYMQLS |
| | | SLTSEDSAVYYCARSTYYGGDWYFNVWGAGTTVTVSAASTKG |
| | | PSVFPLAPSSKSTSGGTAALGCLVKDYFPEPVTVSWNSGALTS |
| | | GVHTFPAVLQSSGLYSLSSVVTVPSSSLGTQTYICNVNHKPSN |
| | | TKVDKRVEPKSCDKTHTCPPCPAPELLGGPSVFLFPPKPKDTL |
| | | MISRTPEVTCVVVDVSHEDPEVKFNWYVDGVEVHNAKTKPRE |
| | | EQYASTYRVVSVLTVLHQDWLNGKEYKCKVSNKALPAPIEKTIS |
| | | KAKGQPREPQVYTLPPSREEMTKNQVSLTCLVKGFYPSDIAVE |
| | | WESNGQPENNYKTTPPVLDSDGSFFLYSRLTVDKSRWQQGN |
| | | VFSCSVMHEALHNHYTQKSLSLSPGK |
| CD3 light chain | 701 | QAVVTQEPSLTVSPGGTVTLTCRSSTGAVTTSNYANWVQQKP |
| | | GQAPRGLIGGTNKRAPWTPARFSGSLLGDKAALTLSGAQPED |
| | | EAEYFCALWYSNLWVFGGGTKLTVLGQPKAAPSVTLFPPSSE |
| | | ELQANKATLVCLISDFYPGAVTVAWKADSSPVKAGVETTTPSK |
| | | QSNNKYAASSYLSLTPEQWKSHRSYSCQVTHEGSTVEKTVAP |
| | | TECS |
| CD3 heavy | 702 | EVQLVESGGGLVQPGGSLKLSCAASGFTFNTYAMNWVRQAS |
| chain | | GKGLEWVGRIRSKYNNYATYYADSVKDRFTISRDDSKSTLYLQ |
| | | MNSLKTEDTAVYYCVRHGNFGNSYVSWFAYWGQGTLVTVSS |
| | | ASTKGPSVFPLAPSSKSTSGGTAALGCLVKDYFPEPVTVSWNS |
| | | GALTSGVHTFPAVLQSSGLYSLSSVVTVPSSSLGTQTYICNVN |
| | | HKPSNTKVDKRVEPKSCDKTHTCPPCPAPELLGGPSVFLFPPK |
| | | PKDTLMISRTPEVTCVVVDVSHEDPEVKFNWYVDGVEVHNAK |
| | | TKPREEQYASTYRVVSVLTVLHQDWLNGKEYKCKVSNKALPA |
| | | PIEKTISKAKGQPREPQVYTLPPSREEMTKNQVSLTCLVKGFYP |
| | | SDIAVEWESNGQPENNYKTTPPVLDSDGSFLLYSKLTVDKSRW |
| | | QQGNVFSCSVMHEALHNHYTQKSLSLSPGK |
| CD20-CD19- | | |
| CD3 trispecific | | |
| CD20 light | 703 | QIVLSQSPAILSASPGEKVTMTCRASSSVSYIHWFQQKPGSSP |
| chain | | KPWIYATSNLASGVPVRFSGSGSGTSYSLTISRVEAEDAATYY |
| | | CQQWTSNPPTFGGGTKLEIKRTVAAPSVFIFPPSDEQLKSGTA |
| | | SVVCLLNNFYPREAKVQWKVDNALQSGNSQESVTEQDSKDST |
| | | YSLSSTLTLSKADYEKHKVYACEVTHQGLSSPVTKSFNRGEC |
| CD20 heavy | 705 | QVQLQQPGAELVKPGASVKMSCKASGYTFTSYNMHWVKQTP |
| chain | | GRGLEWIGAIYPGNGDTSYNQKFKGKATLTADKSSSTAYMQLS |
| | | SLTSEDSAVYYCARSTYYGGDWYFNVWGAGTTVTVSAASTKG |
| | | PSVFPLAPSSKSTSGGTAALGCLVKDYFPEPVTVSWNSGALTS |
| | | GVHTFPAVLQSSGLYSLSSVVTVPSSSLGTQTYICNVNHKPSN |
| | | TKVDKRVEPKSCDKTHTCPPCPAPELLGGPSVFLFPPKPKDTL |
| | | MISRTPEVTCVVVDVSHEDPEVKFNWYVDGVEVHNAKTKPRE |
| | | EQYASTYRVVSVLTVLHQDWLNGKEYKCKVSNKALPAPIEKTIS |
| | | KAKGQPREPQVYTLPPSREEMTKNQVSLTCLVKGFYPSDIAVE |
| | | WESNGQPENNYKTTPPVLDSDGSFLLYSKLTVDKSRWQQGN |
| | | VFSCSVMHEALHNHYTQKSLSLSPGK |
| CD3 light chain | 701 | QAVVTQEPSLTVSPGGTVTLTCRSSTGAVTTSNYANWVQQKP |
| | | GQAPRGLIGGTNKRAPVVTPARFSGSLLGDKAALTLSGAQPED |
| | | EAEYFCALWYSNLWVFGGGTKLTVLGQPKAAPSVTLFPPSSE |
| | | ELQANKATLVCLISDFYPGAVTVAWKADSSPVKAGVETTTPSK |
| | | QSNNKYAASSYLSLTPEQWKSHRSYSCQVTHEGSTVEKTVAP |
| | | TECS |
| CD19 light | 706 | EIVMTQSPATLSLSPGERATLSCRASQDISKYLNWYQQKPGQA |
| chain-CD3 | | PRLLIYHTSRLHSGIPARFSGSGSGTDYTLTISSLQPEDFAVYFC |
| heavy chain | | QQGNTLPYTFGQGTKLEIKRTVAAPSVFIFPPSDEQLKSGTASV |
| | | VCLLNNFYPREAKVQWKVDNALQSGNSQESVTEQDSKDSTYS |
| | | LSSTLTLSKADYEKHKVYACEVTHQGLSSPVTKSFNRGECQVQ |
| | | LVESGGGLVQPGGSLKLSCAASGFTFNTYAMNWVRQASGKG |
| | | LEWVGRIRSKYNNYATYYADSVKDRFTISRDDSKSTLYLQMNS |
| | | LKTEDTAVYYCVRHGNFGNSYVSWFAYWGQGTLVTVSSASTK |
| | | GPSVFPLAPSSKSTSGGTAALGCLVKDYFPEPVTVSWNSGALT |
| | | SGVHTFPAVLQSSGLYSLSSVVTVPSSSLGTQTYICNVNHKPS |
| | | NTKVDKRVEPKSCDKTHTCPPCPAPELLGGPSVFLFPPKPKDT |
| | | LMISRTPEVTCVVVDVSHEDPEVKFNWYVDGVEVHNAKTKPR |
| | | EEQYASTYRVVSVLTVLHQDWLNGKEYKCKVSNKALPAPIEKTI |
| | | SKAKGQPREPQVYTLPPSREEMTKNQVSLTCLVKGFYPSDIAV |
| | | EWESNGQPENNYKTTPPVLDSDGSFFLYSRLTVDKSRWQQG |
| | | NVFSCSVMHEALHNHYTQKSLSLSPGK |
| CD19 heavy | 707 | QVQLQESGPGLVKPSETLSLTCTVSGVSLPDYGVSWIRQPPG |
| chain | | KGLEWIGVIWGSETTYYQSSLKSRVTISKDNSKNQVSLKLSSVT |
| | | AADTAVYYCAKHYYYGGSYAMDYWGQGTLVTVSSASTKGPS |
| | | VFPLAPSSKSTSGGTAALGCLVKDYFPEPVTVSWNSGALTSGV |
| | | HTFPAVLQSSGLYSLSSVVTVPSSSLGTQTYICNVNHKPSNTKV |
| | | DKRVEPKSC |

### 8.1.1.2. In Vitro Functional Activity

In vitro functional activity of the bi- and tri-specific antibodies was evaluated with a luciferase based cytotoxicity assay using the ALL cell line NALM-6 (DSMZ, Braunschwieg, Germany) which was transduced to stably express luciferase. Human T Cells isolated from cryopreserved PBMCs were co-cultured at an effector:target ratio of 5:1 with the NALM-6 target cells. Bispecific or trispecific antibodies were added at various concentrations and incubated for 20 hours after which ONE-Glo Luciferase Assay substrate (Promega, Madison, WI, USA) was added. Luminescence was measured for treated and untreated (to provide maximal luminescence signal) wells and specific lysis (%) was determined as 100-(sample luminescence/average maximal luminescence)*100

### 8.1.2. Results

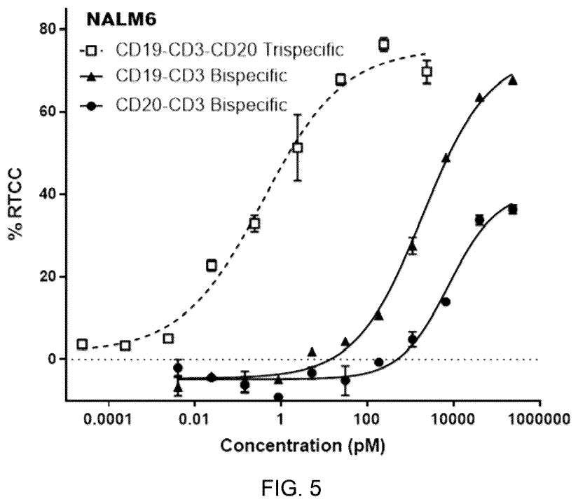

Results of the cytotoxicity assay for the bispecific and trispecific constructs are shown in FIG. 5. All constructs demonstrated cytotoxicity against the CD19^{pos} CD20^{pos} cell line NALM6. The degree of target cell lysis was dependent on antibody concentration and the format of the construct, with the trispecific construct outperforming the bispecific constructs.

### 8.2. Example 2: Production and Characterization of Anti-CD19-Anti-BCMA-Anti-CD3 Human IgG1 Bi- and Tri-Specific Binding Molecules in Knobs-into-Holes Format

### 8.2.1. Gene Construction, Expression and Purification of Bi- and Tri-Specific Binding Molecules

Gene synthesis was performed as described in Example 1. For bispecific constructs, anti-BCMA or anti-CD19 heavy chains was synthesized as fusions of the variable domains to constant hIgG1 domains containing mutations for the hole to facilitate heterodimerization as well as N297A silencing mutation. Light chain plasmids were also synthesized. For the anti-CD3 arm, this was produced as single chain fragment variable fused to constant hIgG1 domains containing mutations for the knob to facilitate heterodimerization as well as N297A silencing mutation. Plasmids for arms containing multi-specificities were also synthesized. Additionally, for these arms plasmids for the heavy chain variable sequence and CH1 sequence for the anti-BCMA arm were also synthesized. Amino acid sequences encoded by the constructs are shown in Table 14.

Bi- and tri-specific antibodies were co-expressed transiently in HEK293 cells. Briefly, transfection was performed using PEI as transfection reagent. For small scale (<5 L) transfections, cells were grown in shake flasks on an orbital shaker (115 rpm) in a humidified incubator (85%) at 5% CO2). Light and heavy chain plasmids for tumor antigen arms were combined with anti-CD3 plasmid with PEI at a final ratio of 1 DNA:3 PEI. 1 mg/L culture of plasmid was used for transfection at 2.0 million cells/mL serum media. After 5 days of expression, the antibody was harvested by clarification of the media via centrifugation and filtration. Purification was performed via anti-CH1 affinity batch binding (CaptureSelect IgG-CH1 Affinity Matrix, Thermo-Fisher Scientific, Waltham, MA, USA) or Protein A (rProteinA Sepharose, Fast flow, GE Healthcare, Uppsala, Sweden) batch binding using 1 ml resin/100 mL supernatant. The protein was allowed to bind for a minimum of 2 hours with gentle mixing, and the supernatant loaded onto a gravity filtration column. The resin was washed with 20-50 CV of PBS. Antibody was eluted with 20 CV of 50 mM citrate, 90 mM NaCl pH 3.2. 50 mM sucrose The eluted IgG protein was adjusted to pH 5.5 with 1 M sodium citrate 50 mM sucrose. If the antibody contained aggregates, preparative size exclusion chromatography was performed using Hi Load 16/60 Superdex 200 grade column (GE Healthcare Life Sciences, Uppsala, Sweden) as a final polishing step. To confirm that the identity of the proteins expressed matched the predicted masses for the primary amino acid sequences shown in Table 14, bispecific and multispecific proteins were analyzed by high-performance liquid chromatography coupled to mass spectrometry.

TABLE 14
Amino acid sequences for binding molecules of Example 2

| Description | SEQ ID No: | Amino acid sequence |
|---|---|---|
| CD19-CD3 | | |
| bispecific (FIG. | | |
| 3A) | | |
| CD19 light | 699 | EIVMTQSPATLSLSPGERATLSCRASQDISKYLNWYQQKPGQA |
| chain | | PRLLIYHTSRLHSGIPARFSGSGSGTDYTLTISSLQPEDFAVYFC |
| | | QQGNTLPYTFGQGTKLEIKRTVAAPSVFIFPPSDEQLKSGTASV |
| | | VCLLNNFYPREAKVQWKVDNALQSGNSQESVTEQDSKDSTYS |
| | | LSSTLTLSKADYEKHKVYACEVTHQGLSSPVTKSFNRGEC |
| CD19 heavy | 708 | QVQLQESGPGLVKPSETLSLTCTVSGVSLPDYGVSWIRQPPG |
| chain | | KGLEWIGVIWGSETTYYQSSLKSRVTISKDNSKNQVSLKLSSVT |
| | | AADTAVYYCAKHYYYGGSYAMDYWGQGTLVTVSSASTKGPS |
| | | VFPLAPSSKSTSGGTAALGCLVKDYFPEPVTVSWNSGALTSGV |
| | | HTFPAVLQSSGLYSLSSWTVPSSSLGTQTYICNVNHKPSNTKV |
| | | DKRVEPKSCDKTHTCPPCPAPELLGGPSVFLFPPKPKDTLMIS |
| | | RTPEVTCVVVDVSHEDPEVKFNWYVDGVEVHNAKTKPREEQY |
| | | ASTYRVVSVLTVLHQDWLNGKEYKCKVSNKALPAPIEKTISKAK |
| | | GQPREPQVCTLPPSREEMTKNQVSLSCAVKGFYPSDIAVEWE |
| | | SNGQPENNYKTTPPVLDSDGSFFLVSKLTVDKSRWQQGNVFS |
| | | CSVMHEALHNHYTQKSLSLSPGK |
| CD3 scFv | 709 | EVQLVESGGGLVQPGGSLKLSCAASGFTFNTYAMNWVRQAS |
| heavy chain | | GKGLEWVGRIRSKYNNYATYYADSVKDRFTISRDDSKSTLYLQ |
| | | MNSLKTEDTAVYYCVRHGNFGNSYVSWFAYWGQGTLVTVSS |
| | | GGGGSGGGGSGGGGSGGGGSQAVVTQEPSLTVSPGGTVTL |
| | | TCRSSTGAVTTSNYANWVQQKPGQAPRGLIGGTNKRAPWTP |
| | | ARFSGSLLGDKAALTLSGAQPEDEAEYFCALWYSNLWVFGGG |
| | | TKLTVLGGGGSDKTHTCPPCPAPELLGGPSVFLFPPKPKDTLM |
| | | ISRTPEVTCVVVDVSHEDPEVKFNWYVDGVEVHNAKTKPREE |
| | | QYASTYRVVSVLTVLHQDWLNGKEYKCKVSNKALPAPIEKTISK |
| | | AKGQPREPQVYTLPPCREEMTKNQVSLWCLVKGFYPSDIAVE |
| | | WESNGQPENNYKTTPPVLDSDGSFFLYSKLTVDKSRWQQGN |
| | | VFSCSVMHEALHNHYTQKSLSLSPGK |
| BCMA-CD3 | | |
| bispecific (FIG. | | |
| 3A) | | |
| BCMA light | 710 | DIQMTQSPSSLSASVGDRVTITCSASQDISNYLNWYQQKPGKA |
| chain | | PKLLIYYTSNLHSGVPSRFSGSGSGTDFTLTISSLQPEDFATYY |
| | | CQQYRKLPWTFGQGTKLEIKRTVAAPSVFIFPPSDEQLKSGTA |
| | | SVVCLLNNFYPREAKVQWKVDNALQSGNSQESVTEQDSKDST |
| | | YSLSSTLTLSKADYEKHKVYACEVTHQGLSSPVTKSFNRGEC |
| BCMA heavy | 711 | QVQLVQSGAEVKKPGSSVKVSCKASGGTFSNYWMHWVRQAP |
| chain | | GQGLEWMGATYRGHSDTYYNQKFKGRVTITADKSTSTAYMEL |
| | | SSLRSEDTAVYYCARGAIYNGYDVLDNWGQGTLVTVSSASTK |
| | | GPSVFPLAPSSKSTSGGTAALGCLVKDYFPEPVTVSWNSGALT |
| | | SGVHTFPAVLQSSGLYSLSSVVTVPSSSLGTQTYICNVNHKPS |
| | | NTKVDKRVEPKSCDKTHTCPPCPAPELLGGPSVFLFPPKPKDT |
| | | LMISRTPEVTCVVVAVSHEDPEVKFNWYVDGVEVHNAKTKPR |
| | | EEQYNSTYRVVSVLTVLHQDWLNGKEYKCKVSNKALAAPIEKTI |
| | | SKAKGQPREPQVCTLPPSREEMTKNQVSLSCAVKGFYPSDIAV |
| | | EWESNGQPENNYKTTPPVLDSDGSFFLVSKLTVDKSRWQQG |
| | | NVFSCSVMHEALHNHYTQKSLSLSPGK |
| CD3 scFv | 712 | EVQLVESGGGLVQPGGSLKLSCAASGFTFNTYAMNWVRQAS |
| heavy chain | | GKGLEWVGRIRSKYNNYATYYADSVKDRFTISRDDSKSTLYLQ |
| | | MNSLKTEDTAVYYCVRHGNFGNSYVSWFAYWGQGTLVTVSS |
| | | GGGGSGGGGSGGGGSGGGGSQAVVTQEPSLTVSPGGTVTL |
| | | TCRSSTGAVTTSNYANWVQQKPGQAPRGLIGGTNKRAPWTP |
| | | ARFSGSLLGDKAALTLSGAQPEDEAEYFCALWYSNLWVFGGG |
| | | TKLTVLGGGGSDKTHTCPPCPAPELLGGPSVFLFPPKPKDTLM |
| | | ISRTPEVTCVVVDVSHEDPEVKFNWYVDGVEVHNAKTKPREE |
| | | QYASTYRVVSVLTVLHQDWLNGKEYKCKVSNKALPAPIEKTISK |
| | | AKGQPREPQVYTLPPCREEMTKQVSLWCLVKGFYPSDIAVEW |
| | | ESNGQPENNYKTTPPVLDSDGSFFLYSKLTVDKSRWQQGNVF |
| | | SCSVMHEALHNHYTQKSLSLSPGK |
| BCMA-CD3- | | |
| CD19 TrisAb 1 | | |
| (FIG. 3B) | | |
| CD19 light | 699 | EIVMTQSPATLSLSPGERATLSCRASQDISKYLNWYQQKPGQA |
| chain | | PRLLIYHTSRLHSGIPARFSGSGSGTDYTLTISSLQPEDFAVYFC |
| | | QQGNTLPYTFGQGTKLEIKRTVAAPSVFIFPPSDEQLKSGTASV |
| | | VCLLNNFYPREAKVQWKVDNALQSGNSQESVTEQDSKDSTYS |
| | | LSSTLTLSKADYEKHKVYACEVTHQGLSSPVTKSFNRGEC |
| CD19 heavy | 708 | QVQLQESGPGLVKPSETLSLTCTVSGVSLPDYGVSWIRQPPG |
| chain | | KGLEWIGVIWGSETTYYQSSLKSRVTISKDNSKNQVSLKLSSVT |
| | | AADTAVYYCAKHYYYGGSYAMDYWGQGTLVTVSSASTKGPS |
| | | VFPLAPSSKSTSGGTAALGCLVKDYFPEPVTVSWNSGALTSGV |
| | | HTFPAVLQSSGLYSLSSVVTVPSSSLGTQTYICNVNHKPSNTKV |
| | | DKRVEPKSCDKTHTCPPCPAPELLGGPSVFLFPPKPKDTLMIS |
| | | RTPEVTCVVVDVSHEDPEVKFNWYVDGVEVHNAKTKPREEQY |
| | | ASTYRVVSVLTVLHQDWLNGKEYKCKVSNKALPAPIEKTISKAK |
| | | GQPREPQVCTLPPSREEMTKNQVSLSCAVKGFYPSDIAVEWE |
| | | SNGQPENNYKTTPPVLDSDGSFFLVSKLTVDKSRWQQGNVFS |
| | | CSVMHEALHNHYTQKSLSLSPGK |
| BCMA light | 713 | DIQMTQSPSSLSASVGDRVTITCSASQDISNYLNWYQQKPGKA |
| chain-CD3 scFv | | PKLLIYYTSNLHSGVPSRFSGSGSGTDFTLTISSLQPEDFATYY |
| heavy chain | | CQQYRKLPWTFGQGTKLEIKRTVAAPSVFIFPPSDEQLKSGTA |
| | | SVVCLLNNFYPREAKVQWKVDNALQSGNSQESVTEQDSKDST |
| | | YSLSSTLTLSKADYEKHKVYACEVTHQGLSSPVTKSFNRGECG |
| | | GGGSGGTGSEVQLVESGGGLVQPGGSLKLSCAASGFTFNTYA |
| | | MNWVRQASGKGLEWVGRIRSKYNNYATYYADSVKDRFTISRD |
| | | DSKSTLYLQMNSLKTEDTAVYYCVRHGNFGNSYVSWFAYWG |
| | | QGTLVTVSSGGGGSGGGGSGGGGSGGGGSQAVVTQEPSLT |
| | | VSPGGTVTLTCRSSTGAVTTSNYANWVQQKPGQAPRGLIGGT |
| | | NKRAPWTPARFSGSLLGDKAALTLSGAQPEDEAEYFCALWYS |
| | | NLWVFGGGTKLTVLSGGGSGGGGSGGGGSGGGGSDKTHTC |
| | | PPCPAPELLGGPSVFLFPPKPKDTLMISRTPEVTCVVVDVSHE |
| | | DPEVKFNWYVDGVEVHNAKTKPREEQYASTYRVVSVLTVLHQ |
| | | DWLNGKEYKCKVSNKALPAPIEKTISKAKGQPREPQVYTLPPC |
| | | REEMTKNQVSLWCLVKGFYPSDIAVEWESNGQPENNYKTTPP |
| | | VLDSDGSFFLYSKLTVDKSRWQQGNVFSCSVMHEALHNRYTQ |
| | | KSLSLSPGK |
| BCMA heavy | 714 | QVQLVQSGAEVKKPGSSVKVSCKASGGTFSNYWMHWVRQAP |
| chain | | GQGLEWMGATYRGHSDTYYNQKFKGRVTITADKSTSTAYMEL |
| | | SSLRSEDTAVYYCARGAIYNGYDVLDNWGQGTLVTVSSASTK |
| | | GPSVFPLAPSSKSTSGGTAALGCLVKDYFPEPVTVSWNSGALT |
| | | SGVHTFPAVLQSSGLYSLSSVVTVPSSSLGTQTYICNVNHKPS |
| | | NTKVDKRVEPKSC |
| BCMA-CD3- | | |
| CD19 TrisAb 2a | | |
| (FIG. 30) | | |
| CD19 light | 699 | EIVMTQSPATLSLSPGERATLSCRASQDISKYLNWYQQKPGQA |
| chain | | PRLLIYHTSRLHSGIPARFSGSGSGTDYTLTISSLQPEDFAVYFC |
| | | QQGNTLPYTFGQGTKLEIKRTVAAPSVFIFPPSDEQLKSGTASV |
| | | VCLLNNFYPREAKVQWKVDNALQSGNSQESVTEQDSKDSTYS |
| | | LSSTLTLSKADYEKHKVYACEVTHQGLSSPVTKSFNRGEC |
| BCMA-Light | 715 | DIQMTQSPSSLSASVGDRVTITCSASQDISNYLNWYQQKPGKA |
| chain-CD19 | | PKLLIYYTSNLHSGVPSRFSGSGSGTDFTLTISSLQPEDFATYY |
| heavy chain | | CQQYRKLPWTFGQGTKLEIKRTVAAPSVFIFPPSDEQLKSGTA |
| | | SVVCLLNNFYPREAKVQWKVDNALQSGNSQESVTEQDSKDST |
| | | YSLSSTLTLSKADYEKHKVYACEVTHQGLSSPVTKSFNRGECG |
| | | GGGSGGGGSQVQLQESGPGLVKPSETLSLTCTVSGVSLPDYG |
| | | VSWIRQPPGKGLEWIGVIWGSETTYYQSSLKSRVTISKDNSKN |
| | | QVSLKLSSVTAADTAVYYCAKHYYYGGSYAMDYWGQGTLVTV |
| | | SSASTKGPSVFPLAPSSKSTSGGTAALGCLVKDYFPEPVTVSW |
| | | NSGALTSGVHTFPAVLQSSGLYSLSSVVTVPSSSLGTQTYICNV |
| | | NHKPSNTKVDKRVEPKSCDKTHTCPPCPAPELLGGPSVFLFPP |
| | | KPKDTLMISRTPEVTCVVVDVSHEDPEVKFNWYVDGVEVHNA |
| | | KTKPREEQYASTYRVVSVLTVLHQDWLNGKEYKCKVSNKALP |
| | | APIEKTISKAKGQPREPQVCTLPPSREEMTKNQVSLSCAVKGF |
| | | YPSDIAVEWESNGQPENNYKTTPPVLDSDGSFFLVSKLTVDKS |
| | | RWQQGNVFSCSVMHEALHNHYTQKSLSLSPGK |
| BCMA Heavy | 714 | QVQLVQSGAEVKKPGSSVKVSCKASGGTFSNYWMHWVRQAP |
| chain | | GQGLEWMGATYRGHSDTYYNQKFKGRVTITADKSTSTAYMEL |
| | | SSLRSEDTAVYYCARGAIYNGYDVLDNWGQGTLVTVSSASTK |
| | | GPSVFPLAPSSKSTSGGTAALGCLVKDYFPEPVTVSWNSGALT |
| | | SGVHTFPAVLQSSGLYSLSSVVTVPSSSLGTQTYICNVNHKPS |
| | | NTKVDKRVEPKSC |
| CD3 scFv | 716 | EVQLVESGGGLVQPGGSLKLSCAASGFTFNTYAMNWVRQAS |
| heavy chain | | GKGLEWVGRIRSKYNNYATYYADSVKDRFTISRDDSKSTLYLQ |
| | | MNSLKTEDTAVYYCVRHGNFGNSYVSWFAYWGQGTLVTVSS |
| | | GGGGSGGGGSGGGGSGGGGSQAVVTQEPSLTVSPGGTVTL |
| | | TCRSSTGAVTTSNYANWVQQKPGQAPRGLIGGTNKRAPWTP |
| | | ARFSGSLLGDKAALTLSGAQPEDEAEYFCALWYSNLWVFGGG |
| | | TKLTVLGGGGSDKTHTCPPCPAPELLGGPSVFLFPPKPKDTLM |
| | | ISRTPEVTCVVVDVSHEDPEVKFNWYVDGVEVHNAKTKPREE |
| | | QYASTYRVVSVLTVLHQDWLNGKEYKCKVSNKALPAPIEKTISK |
| | | AKGQPREPQVYTLPPCREEMTKNQVSLWCLVKGFYPSDIAVE |
| | | WESNGQPENNYKTTPPVLDSDGSFFLYSKLTVDKSRWQQGN |
| | | VFSCSVMHEALHNRYTQKSLSLSPGK |
| BCMA-CD3- | | |
| CD19 TrisAb 2b | | |
| (FIG. 3E) | | |
| CD19 light | 699 | EIVMTQSPATLSLSPGERATLSCRASQDISKYLNWYQQKPGQA |
| chain | | PRLLIYHTSRLHSGIPARFSGSGSGTDYTLTISSLQPEDFAVYFC |
| | | QQGNTLPYTFGQGTKLEIKRTVAAPSVFIFPPSDEQLKSGTASV |
| | | VCLLNNFYPREAKVQWKVDNALQSGNSQESVTEQDSKDSTYS |
| | | LSSTLTLSKADYEKHKVYACEVTHQGLSSPVTKSFNRGEC |
| CD19 heavy | 717 | QVQLQESGPGLVKPSETLSLTCTVSGVSLPDYGVSWIRQPPG |
| chain-BCMA- | | KGLEWIGVIWGSETTYYQSSLKSRVTISKDNSKNQVSLKLSSVT |
| Light chain- | | AADTAVYYCAKHYYYGGSYAMDYWGQGTLVTVSSASTKGPS |
| hIgG1 | | VFPLAPSSKSTSGGTAALGCLVKDYFPEPVTVSWNSGALTSGV |
| | | HTFPAVLQSSGLYSLSSVVTVPSSSLGTQTYICNVNHKPSNTKV |
| | | DKRVEPKSCGGGGSGGGGSDIQMTQSPSSLSASVGDRVTITC |
| | | SASQDISNYLNWYQQKPGKAPKLLIYYTSNLHSGVPSRFSGSG |
| | | SGTDFTLTISSLQPEDFATYYCQQYRKLPWTFGQGTKLEIKRTV |
| | | AAPSVFIFPPSDEQLKSGTASVVCLLNNFYPREAKVQWKVDNA |
| | | LQSGNSQESVTEQDSKDSTYSLSSTLTLSKADYEKHKVYACEV |
| | | THQGLSSPVTKSFNRGECGGGGSDKTHTCPPCPAPELLGGPS |
| | | VFLFPPKPKDTLMISRTPEVTCVVVAVSHEDPEVKFNWYVDGV |
| | | EVHNAKTKPREEQYNSTYRVVSVLTVLHQDWLNGKEYKCKVS |
| | | NKALAAPIEKTISKAKGQPREPQVCTLPPSRDELTKNQVSLSCA |
| | | VKGFYPSDIAVEWESNGQPENNYKTTPPVLDSDGSFFLVSKLT |
| | | VDKSRWQQGNVFSCSVMHEALHNHYTQKSLSLSPGK |
| BCMA Heavy | 714 | QVQLVQSGAEVKKPGSSVKVSCKASGGTFSNYWMHWVRQAP |
| chain | | GQGLEWMGATYRGHSDTYYNQKFKGRVTITADKSTSTAYMEL |
| | | SSLRSEDTAVYYCARGAIYNGYDVLDNWGQGTLVTVSSASTK |
| | | GPSVFPLAPSSKSTSGGTAALGCLVKDYFPEPVTVSWNSGALT |
| | | SGVHTFPAVLQSSGLYSLSSVVTVPSSSLGTQTYICNVNHKPS |
| | | NTKVDKRVEPKSC |
| CD3 scFv | 716 | EVQLVESGGGLVQPGGSLKLSCAASGFTFNTYAMNWVRQAS |
| heavy chain | | GKGLEWVGRIRSKYNNYATYYADSVKDRFTISRDDSKSTLYLQ |
| | | MNSLKTEDTAVYYCVRHGNFGNSYVSWFAYWGQGTLVTVSS |
| | | GGGGSGGGGSGGGGSGGGGSQAVVTQEPSLTVSPGGTVTL |
| | | TCRSSTGAVTTSNYANWVQQKPGQAPRGLIGGTNKRAPWTP |
| | | ARFSGSLLGDKAALTLSGAQPEDEAEYFCALWYSNLWVFGGG |
| | | TKLTVLGGGGSDKTHTCPPCPAPELLGGPSVFLFPPKPKDTLM |
| | | ISRTPEVTCVVVDVSHEDPEVKFNWYVDGVEVHNAKTKPREE |
| | | QYASTYRVVSVLTVLHQDWLNGKEYKCKVSNKALPAPIEKTISK |
| | | AKGQPREPQVYTLPPCREEMTKNQVSLWCLVKGFYPSDIAVE |
| | | WESNGQPENNYKTTPPVLDSDGSFFLYSKLTVDKSRWQQGN |
| | | VFSCSVMHEALHNRYTQKSLSLSPGK |
| BCMA-CD3- | | |
| CD19 TrisAb 3a | | |
| (FIG. 3D) | | |
| CD19 light | 699 | EIVMTQSPATLSLSPGERATLSCRASQDISKYLNWYQQKPGQA |
| chain | | PRLLIYHTSRLHSGIPARFSGSGSGTDYTLTISSLQPEDFAVYFC |
| | | QQGNTLPYTFGQGTKLEIKRTVAAPSVFIFPPSDEQLKSGTASV |
| | | VCLLNNFYPREAKVQWKVDNALQSGNSQESVTEQDSKDSTYS |
| | | LSSTLTLSKADYEKHKVYACEVTHQGLSSPVTKSFNRGEC |
| BCMA-Light | 718 | DIQMTQSPSSLSASVGDRVTITCSASQDISNYLNWYQQKPGKA |
| chain-hIgG1 | | PKLLIYYTSNLHSGVPSRFSGSGSGTDFTLTISSLQPEDFATYY |
| | | CQQYRKLPWTFGQGTKLEIKRTVAAPSVFIFPPSDEQLKSGTA |
| | | SVVCLLNNFYPREAKVQWKVDNALQSGNSQESVTEQDSKDST |
| | | YSLSSTLTLSKADYEKHKVYACEVTHQGLSSPVTKSFNRGECG |
| | | SDKTHTCPPCPAPELLGGPSVFLFPPKPKDTLMISRTPEVTCVV |
| | | VAVSHEDPEVKFNWYVDGVEVHNAKTKPREEQYNSTYRVVSV |
| | | LTVLHQDWLNGKEYKCKVSNKALAAPIEKTISKAKGQPREPQV |
| | | CTLPPSRDELTKNQVSLSCAVKGFYPSDIAVEWESNGQPENN |
| | | YKTTPPVLDSDGSFFLVSKLTVDKSRWQQGNVFSCSVMHEAL |
| | | HNHYTQKSLSLSPGK |
| BCMA Heavy | 714 | QVQLVQSGAEVKKPGSSVKVSCKASGGTFSNYWMHWVRQAP |
| chain | | GQGLEWMGATYRGHSDTYYNQKFKGRVTITADKSTSTAYMEL |
| | | SSLRSEDTAVYYCARGAIYNGYDVLDNWGQGTLVTVSSASTK |
| | | GPSVFPLAPSSKSTSGGTAALGCLVKDYFPEPVTVSWNSGALT |
| | | SGVHTFPAVLQSSGLYSLSSVVTVPSSSLGTQTYICNVNHKPS |
| | | NTKVDKRVEPKSC |
| CD3 scFv | 719 | EVQLVESGGGLVQPGGSLKLSCAASGFTFNTYAMNWVRQAS |
| heavy chain- | | GKGLEWVGRIRSKYNNYATYYADSVKDRFTISRDDSKSTLYLQ |
| hIgG1-CD19 | | MNSLKTEDTAVYYCVRHGNFGNSYVSWFAYWGQGTLVTVSS |
| heavy chain | | GGGGSGGGGSGGGGSGGGGSQAVVTQEPSLTVSPGGTVTL |
| | | TCRSSTGAVTTSNYANWVQQKPGQAPRGLIGGTNKRAPWTP |
| | | ARFSGSLLGDKAALTLSGAQPEDEAEYFCALWYSNLWVFGGG |
| | | TKLTVLGGGGSDKTHTCPPCPAPELLGGPSVFLFPPKPKDTLM |
| | | ISRTPEVTCVVVAVSHEDPEVKFNWYVDGVEVHNAKTKPREE |
| | | QYNSTYRVVSVLTVLHQDWLNGKEYKCKVSNKALAAPIEKTISK |
| | | AKGQPREPQVYTLPPCREEMTKNQVSLWCLVKGFYPSDIAVE |
| | | WESNGQPENNYKTTPPVLDSDGSFFLYSKLTVDKSRWQQGN |
| | | VFSCSVMHEALHNHYTQKSLSLSPGKGGGGSGGGGSGGGGS |
| | | GGGGSQVQLQESGPGLVKPSETLSLTCTVSGVSLPDYGVSWI |
| | | RQPPGKGLEWIGVIWGSETTYYQSSLKSRVTISKDNSKNQVSL |
| | | KLSSVTAADTAVYYCAKHYYYGGSYAMDYWGQGTLVTVSSAS |
| | | TKGPSVFPLAPSSKSTSGGTAALGCLVKDYFPEPVTVSWNSG |
| | | ALTSGVHTFPAVLQSSGLYSLSSVVTVPSSSLGTQTYICNVNHK |
| | | PSNTKVDKRVEPKSC |
| BCMA-CD3- | | |
| CD19 TrisAb 3b | | |
| (FIG. 3F) | | |
| CD19 light | 699 | EIVMTQSPATLSLSPGERATLSCRASQDISKYLNWYQQKPGQA |
| chain | | PRLLIYHTSRLHSGIPARFSGSGSGTDYTLTISSLQPEDFAVYFC |
| | | QQGNTLPYTFGQGTKLEIKRTVAAPSVFIFPPSDEQLKSGTASV |
| | | VCLLNNFYPREAKVQWKVDNALQSGNSQESVTEQDSKDSTYS |
| | | LSSTLTLSKADYEKHKVYACEVTHQGLSSPVTKSFNRGEC |
| CD19 heavy | 708 | QVQLQESGPGLVKPSETLSLTCTVSGVSLPDYGVSWIRQPPG |
| chain | | KGLEWIGVIWGSETTYYQSSLKSRVTISKDNSKNQVSLKLSSVT |
| | | AADTAVYYCAKHYYYGGSYAMDYWGQGTLVTVSSASTKGPS |
| | | VFPLAPSSKSTSGGTAALGCLVKDYFPEPVTVSWNSGALTSGV |
| | | HTFPAVLQSSGLYSLSSVVTVPSSSLGTQTYICNVNHKPSNTKV |
| | | DKRVEPKSCDKTHTCPPCPAPELLGGPSVFLFPPKPKDTLMIS |
| | | RTPEVTCVVVDVSHEDPEVKFNWYVDGVEVHNAKTKPREEQY |
| | | ASTYRVVSVLTVLHQDWLNGKEYKCKVSNKALPAPIEKTISKAK |
| | | GQPREPQVCTLPPSREEMTKNQVSLSCAVKGFYPSDIAVEWE |
| | | SNGQPENNYKTTPPVLDSDGSFFLVSKLTVDKSRWQQGNVFS |
| | | CSVMHEALHNHYTQKSLSLSPGK |
| BCMA Heavy | 714 | QVQLVQSGAEVKKPGSSVKVSCKASGGTFSNYWMHWVRQAP |
| chain | | GQGLEWMGATYRGHSDTYYNQKFKGRVTITADKSTSTAYMEL |
| | | SSLRSEDTAVYYCARGAIYNGYDVLDNWGQGTLVTVSSASTK |
| | | GPSVFPLAPSSKSTSGGTAALGCLVKDYFPEPVTVSWNSGALT |
| | | SGVHTFPAVLQSSGLYSLSSVVTVPSSSLGTQTYICNVNHKPS |
| | | NTKVDKRVEPKSC |
| CD3 scFv | 720 | EVQLVESGGGLVQPGGSLKLSCAASGFTFNTYAMNWVRQAS |
| heavy chain- | | GKGLEWVGRIRSKYNNYATYYADSVKDRFTISRDDSKSTLYLQ |
| hIgG1-BCMA | | MNSLKTEDTAVYYCVRHGNFGNSYVSWFAYWGQGTLVTVSS |
| light chain | | GGGGSGGGGSGGGGSGGGGSQAVVTQEPSLTVSPGGTVTL |
| | | TCRSSTGAVTTSNYANWVQQKPGQAPRGLIGGTNKRAPWTP |
| | | ARFSGSLLGDKAALTLSGAQPEDEAEYFCALWYSNLWVFGGG |
| | | TKLTVLGGGGSDKTHTCPPCPAPELLGGPSVFLFPPKPKDTLM |
| | | ISRTPEVTCVVVDVSHEDPEVKFNWYVDGVEVHNAKTKPREE |
| | | QYASTYRVVSVLTVLHQDWLNGKEYKCKVSNKALPAPIEKTISK |
| | | AKGQPREPQVYTLPPCREEMTKNQVSLWCLVKGFYPSDIAVE |
| | | WESNGQPENNYKTTPPVLDSDGSFFLYSKLTVDKSRWQQGN |
| | | VFSCSVMHEALHNRYTQKSLSLSPGKGGGGSGGGGSGGGGS |
| | | GGGGSDIQMTQSPSSLSASVGDRVTITCSASQDISNYLNWYQ |
| | | QKPGKAPKLLIYYTSNLHSGVPSRFSGSGSGTDFTLTISSLQPE |
| | | DFATYYCQQYRKLPWTFGQGTKLEIKRTVAAPSVFIFPPSDEQ |
| | | LKSGTASVVCLLNNFYPREAKVQWKVDNALQSGNSQESVTEQ |
| | | DSKDSTYSLSSTLTLSKADYEKHKVYACEVTHQGLSSPVTKSF |
| | | NRGEC |
| BCMA-CD3- | | |
| CD19 TrisAb 3c | | |
| (FIG. 3G) | | |
| BCMA light | 710 | DIQMTQSPSSLSASVGDRVTITCSASQDISNYLNWYQQKPGKA |
| chain | | PKLLIYYTSNLHSGVPSRFSGSGSGTDFTLTISSLQPEDFATYY |
| | | CQQYRKLPWTFGQGTKLEIKRTVAAPSVFIFPPSDEQLKSGTA |
| | | SVVCLLNNFYPREAKVQWKVDNALQSGNSQESVTEQDSKDST |
| | | YSLSSTLTLSKADYEKHKVYACEVTHQGLSSPVTKSFNRGEC |
| BCMA heavy | 711 | QVQLVQSGAEVKKPGSSVKVSCKASGGTFSNYWMHWVRQAP |
| chain | | GQGLEWMGATYRGHSDTYYNQKFKGRVTITADKSTSTAYMEL |
| | | SSLRSEDTAVYYCARGAIYNGYDVLDNWGQGTLVTVSSASTK |
| | | GPSVFPLAPSSKSTSGGTAALGCLVKDYFPEPVTVSWNSGALT |
| | | SGVHTFPAVLQSSGLYSLSSVVTVPSSSLGTQTYICNVNHKPS |
| | | NTKVDKRVEPKSCDKTHTCPPCPAPELLGGPSVFLFPPKPKDT |
| | | LMISRTPEVTCVVVAVSHEDPEVKFNWYVDGVEVHNAKTKPR |
| | | EEQYNSTYRVVSVLTVLHQDWLNGKEYKCKVSNKALAAPIEKTI |
| | | SKAKGQPREPQVCTLPPSREEMTKNQVSLSCAVKGFYPSDIAV |
| | | EWESNGQPENNYKTTPPVLDSDGSFFLVSKLTVDKSRWQQG |
| | | NVFSCSVMHEALHNHYTQKSLSLSPGK |
| CD3 scFv | 721 | EVQLVESGGGLVQPGGSLKLSCAASGFTFNTYAMNWVRQAS |
| heavy chain- | | GKGLEWVGRIRSKYNNYATYYADSVKDRFTISRDDSKSTLYLQ |
| hIgG1-CD19 | | MNSLKTEDTAVYYCVRHGNFGNSYVSWFAYWGQGTLVTVSS |
| scFv | | GGGGSGGGGSGGGGSGGGGSQAVVTQEPSLTVSPGGTVTL |
| | | TCRSSTGAVTTSNYANWVQQKPGQAPRGLIGGTNKRAPWTP |
| | | ARFSGSLLGDKAALTLSGAQPEDEAEYFCALWYSNLWVFGGG |
| | | TKLTVLGGGGSDKTHTCPPCPAPELLGGPSVFLFPPKPKDTLM |
| | | ISRTPEVTCVVVAVSHEDPEVKFNWYVDGVEVHNAKTKPREE |
| | | QYNSTYRVVSVLTVLHQDWLNGKEYKCKVSNKALAAPIEKTISK |
| | | AKGQPREPQVYTLPPCREEMTKNQVSLWCLVKGFYPSDIAVE |
| | | WESNGQPENNYKTTPPVLDSDGSFFLYSKLTVDKSRWQQGN |
| | | VFSCSVMHEALHNHYTQKSLSLSPGKGGGGSGGGGSGGGGS |
| | | GGGGSQVQLQESGPGLVKPSETLSLTCTVSGVSLPDYGVSWI |
| | | RQPPGKGLEWIGVIWGSETTYYQSSLKSRVTISKDNSKNQVSL |
| | | KLSSVTAADTAVYYCAKHYYYGGSYAMDYWGQGTLVTVSSG |
| | | GGGSGGGGSGGGGSGGGGSEIVMTQSPATLSLSPGERATLS |
| | | CRASQDISKYLNWYQQKPGQAPRLLIYHTSRLHSGIPARFSGS |
| | | GSGTDYTLTISSLQPEDFAVYFCQQGNTLPYTFGQGTKLEIK |

### 8.2.2. In Vitro Functional Activity

In vitro functional activity was evaluated with a luciferase based cytotoxicity assay using cancer cell lines with either BCMA or CD19 expression. A MM cell line MM1s and a B-ALL cell line Nalm-6 were used as target cells. Both cell lines were transduced to stably express luciferase. In brief, target cells were harvested and incubated with a serial dilution of the antibodies on a 384-well flat-bottom microtiter plate at 10,000 cells per well. Expanded T cells isolated from human PBMC were added to the plate at effector to target ratios of 6:1. An isotype control bispecific Ab (with a “mock” tumor-Ag arm that does not bind to tumor cells) was included as negative control. Following the co-incubation, Bright Glo (Promega) was added to all wells and the luminescence signal was subsequently measured on an Envision (Perkin Elmer). The percent RTCC of target cells was calculated using the following formula: (100−(sample/maximal signal)*100)%]. Maximal luminescence signals were measured from target cell alone (no Ab or T cell added).

All tri-specific antibodies demonstrated robust cytotoxicity against both the BCMA^{pos}CD19^{neg} MM cell line MM1s and the BCMA^{neg} CD19^{pos} B-ALL cell line NALM6 (FIG. 6). All of the BCMA-CD19-CD3 trispecific Ab mediated RTCC of both cell lines by T cells. Surprisingly, BCMA-CD19-CD3 trispecific Ab 3b displayed greater potency compared to CD19-CD3 bispecific antibody to lysis of NALM6 cells. The degree of target cell lysis was dependent on antibody concentration and format. As expected, the negative control antibody was inactive except at high concentrations (>1 nM and about 100-fold lower than the least active bi- or tri-specific Ab), confirming that RTCC requires specific binding of the antibodies to antigens expressed on target cells. The BCMA-CD3 bispecific antibody specifically killed BCMA^{pos}CD19^{neg} MM1s cells, whereas the CD19-CD3 bispecific antibody was highly selective for the BCMA^{neg} CD19^{pos} NALM6 cells.

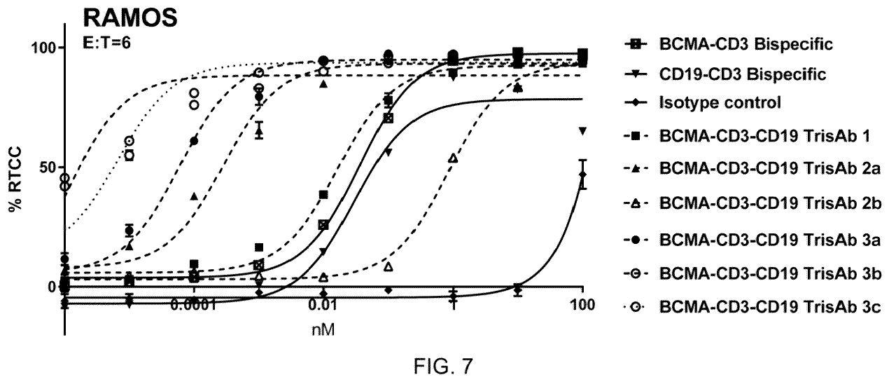

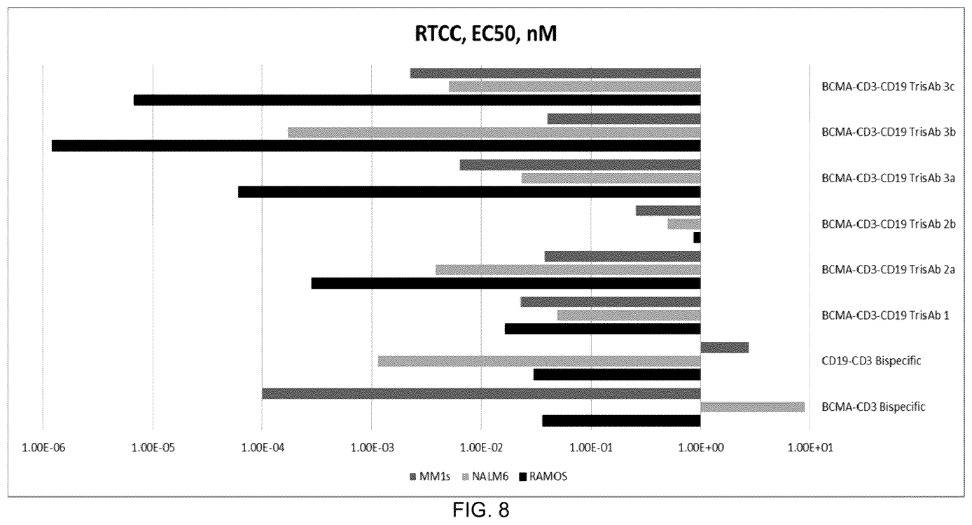

The ability of the bi- and tri-specific antibodies to target malignant B cells expressing both BCMA and CD19 was then tested. Non-Hodgkin lymphoma cell line Ramos was used as target cells and BCMA and CD19 expression was first validated by flow cytometry prior to the assay (data not shown). Cytotoxicity was measured by incubating Ramos cells with expanded T cells at the indicated effector to target ratio in the presence of bi- and tri-specific antibodies. All of the bi- and tri-specific antibodies efficiently mediated lysis of Ramos cells with trispecific antibodies showing superior activity (FIG. 7). In particular, the BCMA-CD3-CD19 trispecific antibodies 3b and 3c demonstrated a striking boost in tumor cell lysis (with >1000-fold higher potency as evidenced by the EC50 shift), which indicated a synergistic effect of simultaneously targeting both antigens (FIG. 8).

### 8.3. Example 3: Production and Characterization of Anti-CD138-Anti-BCMA-Anti-CD3 Human IgG1 Bi- and Tri-Specific Binding Molecules

### 8.3.1. Gene Construction, Expression and Purification of Bi- and Tri-Specific Binding Molecules

Gene synthesis was performed as described in Example 1. For bispecific constructs, anti-BCMA or anti-CD138 heavy chains were synthesized as fusions of the variable domains to constant hIgG1 domains containing mutations for the hole to facilitate hetero-dimerization as well as N297A silencing mutation. Light chain plasmids were synthesized. For the anti-CD3 arm, this was produced as single chain fragment variable fused to constant hIgG1 domains containing mutations for the knob to facilitate heterodimerization as well as N297A silencing mutation. Plasmids for arms containing multi-specificities were synthesized, whereby the light chain of anti-BCMA was fused to the scFv of the CD3 binder with a peptide linker followed by human Fc domain. Additionally for this arm plasmid for the heavy chain variable sequence and CH1 sequence for the anti-BCMA arm was also synthesized. Amino acid sequences encoded by the constructs are shown in Table 15.

Bi- and tri-specific antibodies were co-expressed transiently in HEK293 cells as described in Example 1. To confirm that the identity of the proteins expressed matched the predicted masses for the primary amino acid sequences shown in Table 15, bispecific and multispecific proteins were analyzed by high-performance liquid chromatography coupled to mass spectrometry.

TABLE 15
Amino acid sequences for binding molecules of Example 3

| | SEQ | |
|---|---|---|
| Description | ID No: | Amino acid sequence |
| CD138-CD3 | | |
| bispecific | | |
| CD138 light | 722 | DIQMTQSTSSLSASLGDRVTISCSASQGINNYLNWYQQKPDGT |
| chain | | VELLIYYTSTLQSGVPSRFSGSGSGTDYSLTISNLEPEDIGTYYC |
| | | QQYSKLPRTFGGGTKLEIKRTVAAPSVFIFPPSDEQLKSGTASV |
| | | VCLLNNFYPREAKVQWKVDNALQSGNSQESVTEQDSKDSTYS |
| | | LSSTLTLSKADYEKHKVYACEVTHQGLSSPVTKSFNRGEC |
| CD138 heavy | 723 | QVQLQQSGSELMMPGASVKISCKATGYTFSNYWIEWVKQRPG |
| chain | | HGLEWIGEILPGTGRTIYNEKFKGKATFTADISSNTVQMQLSSL |
| | | TSEDSAVYYCARRDYYGNFYYAMDYWGQGTSVTVSSASTKG |
| | | PSVFPLAPSSKSTSGGTAALGCLVKDYFPEPVTVSWNSGALTS |
| | | GVHTFPAVLQSSGLYSLSSVVTVPSSSLGTQTYICNVNHKPSN |
| | | TKVDKRVEPKSCDKTHTCPPCPAPELLGGPSVFLFPPKPKDTL |
| | | MISRTPEVTCVVVDVSHEDPEVKFNWYVDGVEVHNAKTKPRE |
| | | EQYASTYRVVSVLTVLHQDWLNGKEYKCKVSNKALPAPIEKTIS |
| | | KAKGQPREPQVCTLPPSREEMTKNQVSLSCAVKGFYPSDIAVE |
| | | WESNGQPENNYKTTPPVLDSDGSFFLVSKLTVDKSRWQQGN |
| | | VFSCSVMHEALHNHYTQKSLSLSPGK |
| CD3 scFv | 716 | EVQLVESGGGLVQPGGSLKLSCAASGFTFNTYAMNWVRQAS |
| heavy chain | | GKGLEWVGRIRSKYNNYATYYADSVKDRFTISRDDSKSTLYLQ |
| | | MNSLKTEDTAVYYCVRHGNFGNSYVSWFAYWGQGTLVTVSS |
| | | GGGGSGGGGSGGGGSGGGGSQAVVTQEPSLTVSPGGTVTL |
| | | TCRSSTGAVTTSNYANWVQQKPGQAPRGLIGGTNKRAPWTP |
| | | ARFSGSLLGDKAALTLSGAQPEDEAEYFCALWYSNLWVFGGG |
| | | TKLTVLGGGGSDKTHTCPPCPAPELLGGPSVFLFPPKPKDTLM |
| | | ISRTPEVTCVVVDVSHEDPEVKFNWYVDGVEVHNAKTKPREE |
| | | QYASTYRVVSVLTVLHQDWLNGKEYKCKVSNKALPAPIEKTISK |
| | | AKGQPREPQVYTLPPCREEMTKNQVSLWCLVKGFYPSDIAVE |
| | | WESNGQPENNYKTTPPVLDSDGSFFLYSKLTVDKSRWQQGN |
| | | VFSCSVMHEALHNRYTQKSLSLSPGK |
| BCMA-CD3 | | |
| bispecific | | |
| BCMA light | 710 | DIQMTQSPSSLSASVGDRVTITCSASQDISNYLNWYQQKPGKA |
| chain | | PKLLIYYTSNLHSGVPSRFSGSGSGTDFTLTISSLQPEDFATYY |
| | | CQQYRKLPWTFGQGTKLEIKRTVAAPSVFIFPPSDEQLKSGTA |
| | | SVVCLLNNFYPREAKVQWKVDNALQSGNSQESVTEQDSKDST |
| | | YSLSSTLTLSKADYEKHKVYACEVTHQGLSSPVTKSFNRGEC |
| BCMA heavy | 711 | QVQLVQSGAEVKKPGSSVKVSCKASGGTFSNYWMHWVRQAP |
| chain | | GQGLEWMGATYRGHSDTYYNQKFKGRVTITADKSTSTAYMEL |
| | | SSLRSEDTAVYYCARGAIYNGYDVLDNWGQGTLVTVSSASTK |
| | | GPSVFPLAPSSKSTSGGTAALGCLVKDYFPEPVTVSWNSGALT |
| | | SGVHTFPAVLQSSGLYSLSSVVTVPSSSLGTQTYICNVNHKPS |
| | | NTKVDKRVEPKSCDKTHTCPPCPAPELLGGPSVFLFPPKPKDT |
| | | LMISRTPEVTCVVVAVSHEDPEVKFNWYVDGVEVHNAKTKPR |
| | | EEQYNSTYRVVSVLTVLHQDWLNGKEYKCKVSNKALAAPIEKTI |
| | | SKAKGQPREPQVCTLPPSREEMTKNQVSLSCAVKGFYPSDIAV |
| | | EWESNGQPENNYKTTPPVLDSDGSFFLVSKLTVDKSRWQQG |
| | | NVFSCSVMHEALHNHYTQKSLSLSPGK |
| CD3 scFv | 712 | EVQLVESGGGLVQPGGSLKLSCAASGFTFNTYAMNWVRQAS |
| heavy chain | | GKGLEWVGRIRSKYNNYATYYADSVKDRFTISRDDSKSTLYLQ |
| | | MNSLKTEDTAVYYCVRHGNFGNSYVSWFAYWGQGTLVTVSS |
| | | GGGGSGGGGSGGGGSGGGGSQAVVTQEPSLTVSPGGTVTL |
| | | TCRSSTGAVTTSNYANWVQQKPGQAPRGLIGGTNKRAPWTP |
| | | ARFSGSLLGDKAALTLSGAQPEDEAEYFCALWYSNLWVFGGG |
| | | TKLTVLGGGGSDKTHTCPPCPAPELLGGPSVFLFPPKPKDTLM |
| | | ISRTPEVTCVVVDVSHEDPEVKFNWYVDGVEVHNAKTKPREE |
| | | QYASTYRVVSVLTVLHQDWLNGKEYKCKVSNKALPAPIEKTISK |
| | | AKGQPREPQVYTLPPCREEMTKQVSLWCLVKGFYPSDIAVEW |
| | | ESNGQPENNYKTTPPVLDSDGSFFLYSKLTVDKSRWQQGNVF |
| | | SCSVMHEALHNHYTQKSLSLSPGK |
| CD138-BCMA- | | |
| CD3 trispecific | | |
| CD138 light | 722 | DIQMTQSTSSLSASLGDRVTISCSASQGINNYLNWYQQKPDGT |
| chain | | VELLIYYTSTLQSGVPSRFSGSGSGTDYSLTISNLEPEDIGTYYC |
| | | QQYSKLPRTFGGGTKLEIKRTVAAPSVFIFPPSDEQLKSGTASV |
| | | VCLLNNFYPREAKVQWKVDNALQSGNSQESVTEQDSKDSTYS |
| | | LSSTLTLSKADYEKHKVYACEVTHQGLSSPVTKSFNRGEC |
| CD138 heavy | 723 | QVQLQQSGSELMMPGASVKISCKATGYTFSNYWIEWVKQRPG |
| chain | | HGLEWIGEILPGTGRTIYNEKFKGKATFTADISSNTVQMQLSSL |
| | | TSEDSAVYYCARRDYYGNFYYAMDYWGQGTSVTVSSASTKG |
| | | PSVFPLAPSSKSTSGGTAALGCLVKDYFPEPVTVSWNSGALTS |
| | | GVHTFPAVLQSSGLYSLSSVVTVPSSSLGTQTYICNVNHKPSN |
| | | TKVDKRVEPKSCDKTHTCPPCPAPELLGGPSVFLFPPKPKDTL |
| | | MISRTPEVTCVVVDVSHEDPEVKFNWYVDGVEVHNAKTKPRE |
| | | EQYASTYRVVSVLTVLHQDWLNGKEYKCKVSNKALPAPIEKTIS |
| | | KAKGQPREPQVCTLPPSREEMTKNQVSLSCAVKGFYPSDIAVE |
| | | WESNGQPENNYKTTPPVLDSDGSFFLVSKLTVDKSRWQQGN |
| | | VFSCSVMHEALHNHYTQKSLSLSPGK |
| BCMA light | 713 | DIQMTQSPSSLSASVGDRVTITCSASQDISNYLNWYQQKPGKA |
| chain-CD3 | | PKLLIYYTSNLHSGVPSRFSGSGSGTDFTLTISSLQPEDFATYY |
| heavy chain | | CQQYRKLPWTFGQGTKLEIKRTVAAPSVFIFPPSDEQLKSGTA |
| | | SVVCLLNNFYPREAKVQWKVDNALQSGNSQESVTEQDSKDST |
| | | YSLSSTLTLSKADYEKHKVYACEVTHQGLSSPVTKSFNRGECG |
| | | GGGSGGTGSEVQLVESGGGLVQPGGSLKLSCAASGFTFNTYA |
| | | MNWVRQASGKGLEWVGRIRSKYNNYATYYADSVKDRFTISRD |
| | | DSKSTLYLQMNSLKTEDTAVYYCVRHGNFGNSYVSWFAYWG |
| | | QGTLVTVSSGGGGSGGGGSGGGGSGGGGSQAVVTQEPSLT |
| | | VSPGGTVTLTCRSSTGAVTTSNYANWVQQKPGQAPRGLIGGT |
| | | NKRAPWTPARFSGSLLGDKAALTLSGAQPEDEAEYFCALWYS |
| | | NLWVFGGGTKLTVLSGGGSGGGGSGGGGSGGGGSDKTHTC |
| | | PPCPAPELLGGPSVFLFPPKPKDTLMISRTPEVTCVVVDVSHE |
| | | DPEVKFNWYVDGVEVHNAKTKPREEQYASTYRVVSVLTVLHQ |
| | | DWLNGKEYKCKVSNKALPAPIEKTISKAKGQPREPQVYTLPPC |
| | | REEMTKNQVSLWCLVKGFYPSDIAVEWESNGQPENNYKTTPP |
| | | VLDSDGSFFLYSKLTVDKSRWQQGNVFSCSVMHEALHNRYTQ |
| | | KSLSLSPGK |
| BCMA heavy | 714 | QVQLVQSGAEVKKPGSSVKVSCKASGGTFSNYWMHWVRQAP |
| chain | | GQGLEWMGATYRGHSDTYYNQKFKGRVTITADKSTSTAYMEL |
| | | SSLRSEDTAVYYCARGAIYNGYDVLDNWGQGTLVTVSSASTK |
| | | GPSVFPLAPSSKSTSGGTAALGCLVKDYFPEPVTVSWNSGALT |
| | | SGVHTFPAVLQSSGLYSLSSVVTVPSSSLGTQTYICNVNHKPS |
| | | NTKVDKRVEPKSC |

### 8.3.1. In Vitro Functional Activity

### 8.3.1.1. Overview

Nearly all MM cells and cell lines evaluated to date also express CD138, which provides a scaffold for APRIL, the ligand for BCMA, and facilitates APRIL-BCMA binding to activate growth and survival signaling pathways. The CD138×BCMA×CD3 trispecific antibody was generated with the goal to simultaneously bind BCMA and CD138 on the surface of MM cells, and more robustly crosslink the TCR complex on T cells for T cell activation.

BCMA is known to be cleaved by γ-secretase at the transmembrane domain, which result in shedding of the extracellular domain (ECD) of BCMA. The serum concentration of shed BCMA ECD positively correlates with tumor load in patients, and has been reported at levels ranging between 100 ng/mL and up to 3,000 ng/mL (median level about 500 ng/mL; Ghermezi et al. Haematologica. 2017.102(4)). The shed BCMA ECD may act as a neutralizing sink for administered BCMA-targeting Ab in patients. Without being bound by theory, it is thought that a CD138×BCMA×CD3 trispecific antibody that can simultaneously engage CD138 and BCMA can provide higher binding avidity to cell surface BCMA, which can translate into greater activity in the presence of high serum levels of shed BCMA. To test this hypothesis, the impact of soluble BCMA ECD on the activity of the BCMA×CD3 bispecific to the CD138×BCMA×CD3 trispecific Ab was compared in an RTCC assay using MM1s, a BCMA+/CD138+MM cell line.

### 8.3.1.2. Materials and Methods

CD138 and BCMA cell surface expression on MM1s was determined by flow cytometry using BV421 labeled anti-BCMA (clone 19F2, Biolegend 357520), and BV711 labeled anti-CD138 (clone M115, Biolegend 356522). Data were acquired on BD LSR Fortessa and analyzed using FlowJo (ver. 10). Delta mean fluorescence intensity (ΔMFI) was determined by subtracting the MFI of unstained cells to that of anti-BCMA-BV421, or anti-CD138-BV510 stained cells.

Human T cells were isolated from peripheral blood of healthy human donors. First, peripheral blood mononuclear cells (PBMCs) were fractionated from donor blood using a Ficoll-Paque PLUS (GE Healthcare #17-1440-02) density gradient. T-cells were then isolated from PBMCs by negative selection according to manufacturer's recommended protocol (Miltenyi #130-096-535). The isolated T-cells were further expanded using Human T-Activator CD3/CD28 Dynabeads (Gibco #11132D) for nine days, then debeaded magnetically and stored as viable frozen aliquots in liquid nitrogen. The expanded T cells were used as effector T cells in RTCC assays where they were thawed from frozen aliquots, counted and used immediately at an Effector:Target (E:T) cell ratio of 3:1.

In vitro RTCC assay: the target MM cell line MM1s was transduced to constitutively express luciferase, which is used to measure cell viability/survival. Target cells were plated at 7,500 cells/well together with 22,500 cells/well effector cells (expanded T-cells) in 384-well plates (Costar 3765) in TCM. TCM is RPMI/1640-based with the addition of 10% FBS, 2 mM L-glutamine, 0.1 mM Non-essential amino acids, 1 mM Sodium pyruvate, 10 mM HEPES, 0.055 mM 2-mercaptoethanol (Gibco 22400089, 16140, 25030-081, 11140-050, 11360-070, 15630-080, 21985-023 respectively). The antibodies were serially diluted, then added to the wells. The assay was incubated at 37 °C/5% CO2 for 20 hr, followed by measurements of luciferase activity to indicate target cell viability (BrightGlo, Promega #E2650) following manufacturer's protocols. Target cells only without T cells or antibodies serve as control for 100% luciferase activity (100% viability). Data were analyzed using Spotfire, where EC50 values were calculated using logistic regression curve fit.

### 8.3.1.3. Results and Discussion

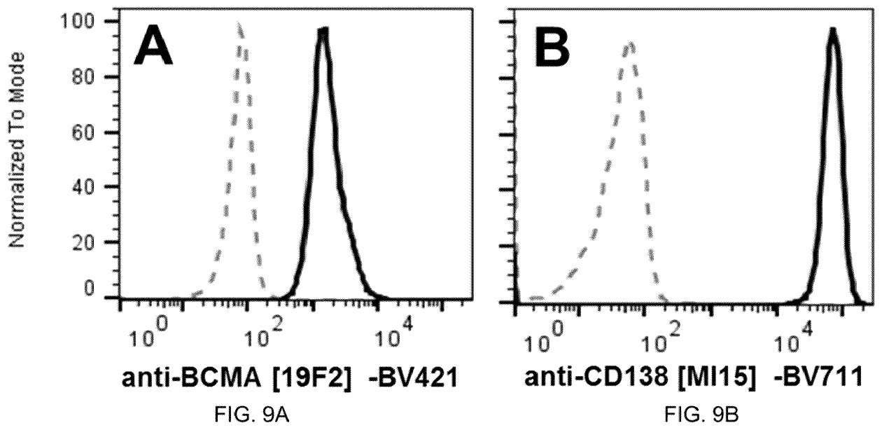

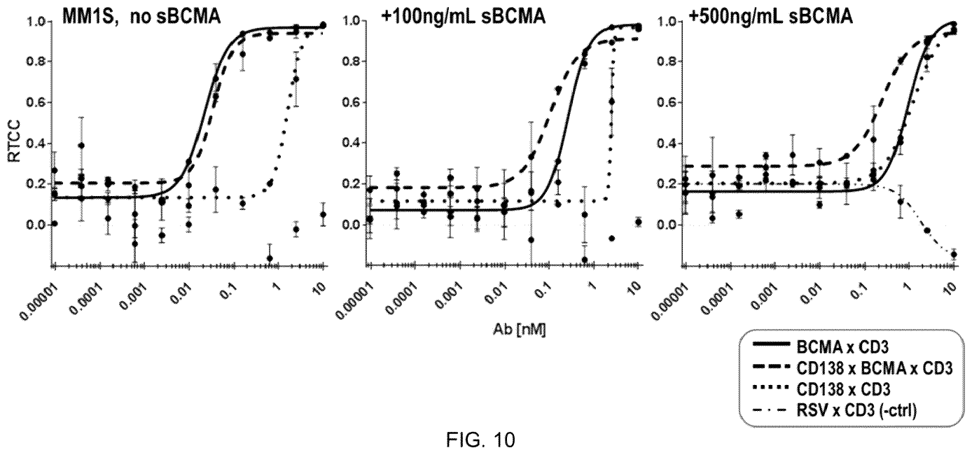

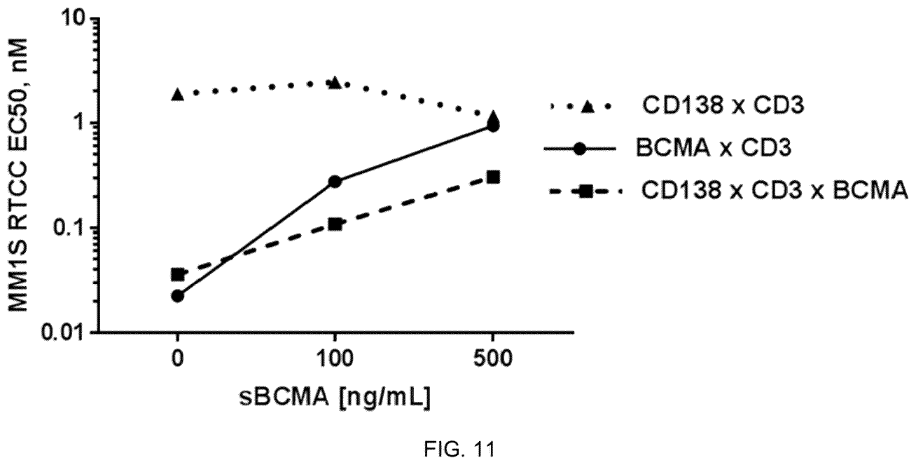

BCMA and CD138 were both expressed on the surface of MM1s cells (FIG. 9). The CD138×BCMA×CD3 trispecific Ab demonstrated similar activity as the BCMA×CD3 bispecific Ab in an RTCC assay on MM1s cells (see, FIG. 10 left panel, no soluble BCMA added). Addition of soluble BCMA ECD into the assay medium reduced the potency of the antibodies, indicated by the higher concentrations needed to mediate MM cell lysis (see, FIG. 10 middle and right panels), and the shift of EC50 to higher values (FIG. 11). The CD138×CD3 bispecific antibody was tested as a control, which mediated RTCC of MM1s albeit at lower levels; and its activity was not affected by the addition of soluble BCMA. Without being bound by theory, it is believed that the relative insensitivity of the CD138×BCMA×CD3 trispecific to soluble BCMA ECD compared to the BCMA×CD3 bispecific shows that a dual-targeting trispecific Ab can provide a better strategy of targeting a cell surface protein that is cleaved and shed significantly from the cell surface. Again without being bound by theory, in the case of BCMA-targeting TCR-engaging multispecific Ab, having an additional ABM that binds to another target on the surface of MM cells is thought to translate into greater clinical activity in patients with high serum levels of shed BCMA.

### 9. SPECIFIC EMBODIMENTS, CITATION OF REFERENCES

While various specific embodiments have been illustrated and described, it will be appreciated that various changes can be made without departing from the spirit and scope of the disclosure(s). The present disclosure is exemplified by the numbered embodiments set forth below.
1. A multispecific binding molecule (MBM), comprising:
   (a) an antigen-binding module 1 (ABM1) that binds specifically to a first human tumor-associated antigen that is expressed on cancerous B cells (TAA 1);
   (b) an antigen-binding module 2 (ABM2) that binds specifically to a second human tumor-associated antigen that is expressed on cancerous B cells (TAA 2); and
   (c) an antigen-binding module 3 (ABM3) that binds specifically to a component of a human T-cell receptor (TCR) complex.
2. The MBM of embodiment 1, wherein TAA 1 is expressed on cancerous B cells that are B cell-derived plasma cells.
3. The MBM of embodiment 1 or embodiment 2, wherein TAA 2 is expressed on cancerous B cells that are B cell-derived plasma cells.
4. The MBM of embodiment 1, wherein TAA 1 is expressed on cancerous B cells that are not plasma cells.
5. The MBM of embodiment 1 or embodiment 4, wherein TAA 2 is expressed on cancerous B cells that are not plasma cells.
6. The MBM of any one of embodiments 1 to 5, wherein TAA 1 and TAA 2 are expressed on the same cancerous B cell.
7. The MBM of any one of embodiments 1 to 5, wherein TAA 1 and TAA 2 are expressed on different cancerous B cells.
8. The MBM of any one of embodiments 1 to 7, wherein each antigen-binding module is capable of binding its respective target at the same time as each of the other antigen-binding modules is bound to its respective target.
9. The MBM of any one of embodiments 1 to 8, wherein TAA 1 and TAA 2 are each independently selected from CD19, CD20, CD22, CD123, BCMA, CD33, CLL1, CD138, CS1, CD38, CD133, FLT3, CD52, TNFRSF13C, TNFRSF13B, CXCR4, PD-L1, LY9, CD200, FCGR2B, CD21, CD23, CD24, CD40L, CD72, CD79a, and CD79b.
10. The MBM of any one of embodiments 1 to 9, wherein TAA 1 is CD19.
11. The MBM of embodiment 10, wherein TAA 2 is CD20.
12. The MBM of embodiment 10, wherein TAA 2 is CD22.
13. The MBM of embodiment 10, wherein TAA 2 is CD123.
14. The MBM of embodiment 10, wherein TAA 2 is BCMA.
15. The MBM of embodiment 10, wherein TAA 2 is CD33.
16. The MBM of embodiment 10, wherein TAA 2 is CLL1.
17. The MBM of embodiment 10, wherein TAA 2 is CD138.
18. The MBM of embodiment 10, wherein TAA 2 is CS1.
19. The MBM of embodiment 10, wherein TAA 2 is CD38.
20. The MBM of embodiment 10, wherein TAA 2 is CD133.
21. The MBM of embodiment 10, wherein TAA 2 is FLT3.
22. The MBM of embodiment 10, wherein TAA 2 is CD52.
23. The MBM of embodiment 10, wherein TAA 2 is TNFRSF13C.
24. The MBM of embodiment 10, wherein TAA 2 is TNFRSF13B.
25. The MBM of embodiment 10, wherein TAA 2 is CXCR4.
26. The MBM of embodiment 10, wherein TAA 2 is PD-L1.
27. The MBM of embodiment 10, wherein TAA 2 is LY9.
28. The MBM of embodiment 10, wherein TAA 2 is CD200.
29. The MBM of embodiment 10, wherein TAA 2 is FCGR2B.
30. The MBM of embodiment 10, wherein TAA 2 is CD21.
31. The MBM of embodiment 10, wherein TAA 2 is CD23.
32. The MBM of embodiment 10, wherein TAA 2 is CD24.
33. The MBM of embodiment 10, wherein TAA 2 is CD40L.
34. The MBM of embodiment 10, wherein TAA 2 is CD72.
35. The MBM of embodiment 10, wherein TAA 2 is CD79a.
36. The MBM of embodiment 10, wherein TAA 2 is CD79b.
37. The MBM of any one of embodiments 1 to 9, wherein TAA 1 is CD20.
38. The MBM of embodiment 37, wherein TAA 2 is CD22.
39. The MBM of embodiment 37, wherein TAA 2 is CD123.
40. The MBM of embodiment 37, wherein TAA 2 is BCMA.
41. The MBM of embodiment 37, wherein TAA 2 is CD33.
42. The MBM of embodiment 37, wherein TAA 2 is CLL1.
43. The MBM of embodiment 37, wherein TAA 2 is CD138.
44. The MBM of embodiment 37, wherein TAA 2 is CS1.
45. The MBM of embodiment 37, wherein TAA 2 is CD38.
46. The MBM of embodiment 37, wherein TAA 2 is CD133.
47. The MBM of embodiment 37, wherein TAA 2 is FLT3.
48. The MBM of embodiment 37, wherein TAA 2 is CD52.
49. The MBM of embodiment 37, wherein TAA 2 is TNFRSF13C.
50. The MBM of embodiment 37, wherein TAA 2 is TNFRSF13B.
51. The MBM of embodiment 37, wherein TAA 2 is CXCR4.
52. The MBM of embodiment 37, wherein TAA 2 is PD-L1.
53. The MBM of embodiment 37, wherein TAA 2 is LY9.
54. The MBM of embodiment 37, wherein TAA 2 is CD200.
55. The MBM of embodiment 37, wherein TAA 2 is FCGR2B.
56. The MBM of embodiment 37, wherein TAA 2 is CD21.
57. The MBM of embodiment 37, wherein TAA 2 is CD23.
58. The MBM of embodiment 37, wherein TAA 2 is CD24.
59. The MBM of embodiment 37, wherein TAA 2 is CD40L.
60. The MBM of embodiment 37, wherein TAA 2 is CD72.
61. The MBM of embodiment 37, wherein TAA 2 is CD79a.
62. The MBM of embodiment 37, wherein TAA 2 is CD79b.
63. The MBM of any one of embodiments 1 to 9, wherein TAA 1 is CD22.
64. The MBM of embodiment 63, wherein TAA 2 is CD123.
65. The MBM of embodiment 63, wherein TAA 2 is BCMA.
66. The MBM of embodiment 63, wherein TAA 2 is CD33.
67. The MBM of embodiment 63, wherein TAA 2 is CLL1.
68. The MBM of embodiment 63, wherein TAA 2 is CD138.
69. The MBM of embodiment 63, wherein TAA 2 is CS1.
70. The MBM of embodiment 63, wherein TAA 2 is CD38.
71. The MBM of embodiment 63, wherein TAA 2 is CD133.
72. The MBM of embodiment 63, wherein TAA 2 is FLT3.
73. The MBM of embodiment 63, wherein TAA 2 is CD52.
74. The MBM of embodiment 63, wherein TAA 2 is TNFRSF13C.
75. The MBM of embodiment 63, wherein TAA 2 is TNFRSF13B.
76. The MBM of embodiment 63, wherein TAA 2 is CXCR4.
77. The MBM of embodiment 63, wherein TAA 2 is PD-L1.
78. The MBM of embodiment 63, wherein TAA 2 is LY9.
79. The MBM of embodiment 63, wherein TAA 2 is CD200.
80. The MBM of embodiment 63, wherein TAA 2 is FCGR2B.
81. The MBM of embodiment 63, wherein TAA 2 is CD21.
82. The MBM of embodiment 63, wherein TAA 2 is CD23.
83. The MBM of embodiment 63, wherein TAA 2 is CD24.
84. The MBM of embodiment 63, wherein TAA 2 is CD40L.
85. The MBM of embodiment 63, wherein TAA 2 is CD72.
86. The MBM of embodiment 63, wherein TAA 2 is CD79a.
87. The MBM of embodiment 63, wherein TAA 2 is CD79b.
88. The MBM of any one of embodiments 1 to 9, wherein TAA 1 is CD123.
89. The MBM of embodiment 88, wherein TAA 2 is BCMA.
90. The MBM of embodiment 88, wherein TAA 2 is CD33.
91. The MBM of embodiment 88, wherein TAA 2 is CLL1.
92. The MBM of embodiment 88, wherein TAA 2 is CD138.
93. The MBM of embodiment 88, wherein TAA 2 is CS1.
94. The MBM of embodiment 88, wherein TAA 2 is CD38.
95. The MBM of embodiment 88, wherein TAA 2 is CD133.
96. The MBM of embodiment 88, wherein TAA 2 is FLT3.
97. The MBM of embodiment 88, wherein TAA 2 is CD52.
98. The MBM of embodiment 88, wherein TAA 2 is TNFRSF13C.
99. The MBM of embodiment 88, wherein TAA 2 is TNFRSF13B.
100. The MBM of embodiment 88, wherein TAA 2 is CXCR4.
101. The MBM of embodiment 88, wherein TAA 2 is PD-L1.
102. The MBM of embodiment 88, wherein TAA 2 is LY9.
103. The MBM of embodiment 88, wherein TAA 2 is CD200.
104. The MBM of embodiment 88, wherein TAA 2 is FCGR2B.
105. The MBM of embodiment 88, wherein TAA 2 is CD21.
106. The MBM of embodiment 88, wherein TAA 2 is CD23.
107. The MBM of embodiment 88, wherein TAA 2 is CD24.
108. The MBM of embodiment 86, wherein TAA 2 is CD40L.
109. The MBM of embodiment 88, wherein TAA 2 is CD72.
110. The MBM of embodiment 88, wherein TAA 2 is CD79a.
111. The MBM of embodiment 88, wherein TAA 2 is CD79b.
112. The MBM of any one of embodiments 1 to 9, wherein TAA 1 is BCMA.
113. The MBM of embodiment 112, wherein TAA 2 is CD33.
114. The MBM of embodiment 112, wherein TAA 2 is CLL1.
115. The MBM of embodiment 112, wherein TAA 2 is CD138.
116. The MBM of embodiment 112, wherein TAA 2 is CS1.
117. The MBM of embodiment 112, wherein TAA 2 is CD38.
118. The MBM of embodiment 112, wherein TAA 2 is CD133.
119. The MBM of embodiment 112, wherein TAA 2 is FLT3.
120. The MBM of embodiment 112, wherein TAA 2 is CD52.
121. The MBM of embodiment 112, wherein TAA 2 is TNFRSF13C.
122. The MBM of embodiment 112, wherein TAA 2 is TNFRSF13B.
123. The MBM of embodiment 112, wherein TAA 2 is CXCR4.
124. The MBM of embodiment 112, wherein TAA 2 is PD-L1.
125. The MBM of embodiment 112, wherein TAA 2 is LY9.
126. The MBM of embodiment 112, wherein TAA 2 is CD200.
127. The MBM of embodiment 112, wherein TAA 2 is FCGR2B.
128. The MBM of embodiment 112, wherein TAA 2 is CD21.
129. The MBM of embodiment 112, wherein TAA 2 is CD23.
130. The MBM of embodiment 112, wherein TAA 2 is CD24.
131. The MBM of embodiment 112, wherein TAA 2 is CD40L.
132. The MBM of embodiment 112, wherein TAA 2 is CD72.
133. The MBM of embodiment 112, wherein TAA 2 is CD79a.
134. The MBM of embodiment 112, wherein TAA 2 is CD79b.
135. The MBM of any one of embodiments 1 to 9, wherein TAA 1 is CD33.
136. The MBM of embodiment 135, wherein TAA 2 is CLL1.
137. The MBM of embodiment 135, wherein TAA 2 is CD138.
138. The MBM of embodiment 135, wherein TAA 2 is CS1.
139. The MBM of embodiment 135, wherein TAA 2 is CD38.
140. The MBM of embodiment 135, wherein TAA 2 is CD133.
141. The MBM of embodiment 135, wherein TAA 2 is FLT3.
142. The MBM of embodiment 135, wherein TAA 2 is CD52.
143. The MBM of embodiment 135, wherein TAA 2 is TNFRSF13C.
144. The MBM of embodiment 135, wherein TAA 2 is TNFRSF13B.
145. The MBM of embodiment 135, wherein TAA 2 is CXCR4.
146. The MBM of embodiment 135, wherein TAA 2 is PD-L1.
147. The MBM of embodiment 135, wherein TAA 2 is LY9.
148. The MBM of embodiment 135, wherein TAA 2 is CD200.
149. The MBM of embodiment 135, wherein TAA 2 is FCGR2B.
150. The MBM of embodiment 135, wherein TAA 2 is CD21.
151. The MBM of embodiment 135, wherein TAA 2 is CD23.
152. The MBM of embodiment 135, wherein TAA 2 is CD24.
153. The MBM of embodiment 135, wherein TAA 2 is CD40L.
154. The MBM of embodiment 135, wherein TAA 2 is CD72.
155. The MBM of embodiment 135, wherein TAA 2 is CD79a.
156. The MBM of embodiment 135, wherein TAA 2 is CD79b.
157. The MBM of any one of embodiments 1 to 9, wherein TAA 1 is CLL1.
158. The MBM of embodiment 157, wherein TAA 2 is CD138.
159. The MBM of embodiment 157, wherein TAA 2 is CS1.
160. The MBM of embodiment 157, wherein TAA 2 is CD38.
161. The MBM of embodiment 157, wherein TAA 2 is CD133.
162. The MBM of embodiment 157, wherein TAA 2 is FLT3.
163. The MBM of embodiment 157, wherein TAA 2 is CD52.
164. The MBM of embodiment 157, wherein TAA 2 is TNFRSF13C.
165. The MBM of embodiment 157, wherein TAA 2 is TNFRSF13B.
166. The MBM of embodiment 157, wherein TAA 2 is CXCR4.
167. The MBM of embodiment 157, wherein TAA 2 is PD-L1.
168. The MBM of embodiment 157, wherein TAA 2 is LY9.
169. The MBM of embodiment 157, wherein TAA 2 is CD200.
170. The MBM of embodiment 157, wherein TAA 2 is FCGR2B.
171. The MBM of embodiment 157, wherein TAA 2 is CD21.
172. The MBM of embodiment 157, wherein TAA 2 is CD23.
173. The MBM of embodiment 157, wherein TAA 2 is CD24.
174. The MBM of embodiment 157, wherein TAA 2 is CD40L.
175. The MBM of embodiment 157, wherein TAA 2 is CD72.
176. The MBM of embodiment 157, wherein TAA 2 is CD79a.
177. The MBM of embodiment 157, wherein TAA 2 is CD79b.
178. The MBM of any one of embodiments 1 to 9, wherein TAA 1 is CD138.
179. The MBM of embodiment 178, wherein TAA 2 is CS1.
180. The MBM of embodiment 178, wherein TAA 2 is CD38.
181. The MBM of embodiment 178, wherein TAA 2 is CD133.
182. The MBM of embodiment 178, wherein TAA 2 is FLT3.
183. The MBM of embodiment 178, wherein TAA 2 is CD52.
184. The MBM of embodiment 178, wherein TAA 2 is TNFRSF13C.
185. The MBM of embodiment 178, wherein TAA 2 is TNFRSF13B.
186. The MBM of embodiment 178, wherein TAA 2 is CXCR4.
187. The MBM of embodiment 178, wherein TAA 2 is PD-L1.
188. The MBM of embodiment 178, wherein TAA 2 is LY9.
189. The MBM of embodiment 178, wherein TAA 2 is CD200.
190. The MBM of embodiment 178, wherein TAA 2 is FCGR2B.
191. The MBM of embodiment 178, wherein TAA 2 is CD21.
192. The MBM of embodiment 178, wherein TAA 2 is CD23.
193. The MBM of embodiment 178, wherein TAA 2 is CD24.
194. The MBM of embodiment 178, wherein TAA 2 is CD40L.
195. The MBM of embodiment 178, wherein TAA 2 is CD72.
196. The MBM of embodiment 178, wherein TAA 2 is CD79a.
197. The MBM of embodiment 178, wherein TAA 2 is CD79b.
198. The MBM of any one of embodiments 1 to 9, wherein TAA 1 is CS1.
199. The MBM of embodiment 198, wherein TAA 2 is CD38.
200. The MBM of embodiment 198, wherein TAA 2 is CD133.
201. The MBM of embodiment 198, wherein TAA 2 is FLT3.
202. The MBM of embodiment 198, wherein TAA 2 is CD52.
203. The MBM of embodiment 198, wherein TAA 2 is TNFRSF13C.
204. The MBM of embodiment 198, wherein TAA 2 is TNFRSF13B.
205. The MBM of embodiment 198, wherein TAA 2 is CXCR4.
206. The MBM of embodiment 198, wherein TAA 2 is PD-L1.
207. The MBM of embodiment 198, wherein TAA 2 is LY9.
208. The MBM of embodiment 198, wherein TAA 2 is CD200.
209. The MBM of embodiment 198, wherein TAA 2 is FCGR2B.
210. The MBM of embodiment 198, wherein TAA 2 is CD21.
211. The MBM of embodiment 198, wherein TAA 2 is CD23.
212. The MBM of embodiment 198, wherein TAA 2 is CD24.
213. The MBM of embodiment 198, wherein TAA 2 is CD40L.
214. The MBM of embodiment 198, wherein TAA 2 is CD72.
215. The MBM of embodiment 198, wherein TAA 2 is CD79a.
216. The MBM of embodiment 198, wherein TAA 2 is CD79b.
217. The MBM of any one of embodiments 1 to 9, wherein TAA 1 is CD38.
218. The MBM of embodiment 217, wherein TAA 2 is CD133.
219. The MBM of embodiment 217, wherein TAA 2 is FLT3.
220. The MBM of embodiment 217, wherein TAA 2 is CD52.
221. The MBM of embodiment 217, wherein TAA 2 is TNFRSF13C.
222. The MBM of embodiment 217, wherein TAA 2 is TNFRSF13B.
223. The MBM of embodiment 217, wherein TAA 2 is CXCR4.
224. The MBM of embodiment 217, wherein TAA 2 is PD-L1.
225. The MBM of embodiment 217, wherein TAA 2 is LY9.
226. The MBM of embodiment 217, wherein TAA 2 is CD200.
227. The MBM of embodiment 217, wherein TAA 2 is FCGR2B.
228. The MBM of embodiment 217, wherein TAA 2 is CD21.
229. The MBM of embodiment 217, wherein TAA 2 is CD23.
230. The MBM of embodiment 217, wherein TAA 2 is CD24.
231. The MBM of embodiment 217, wherein TAA 2 is CD40L.
232. The MBM of embodiment 217, wherein TAA 2 is CD72.
233. The MBM of embodiment 217, wherein TAA 2 is CD79a.
234. The MBM of embodiment 217, wherein TAA 2 is CD79b.
235. The MBM of any one of embodiments 1 to 9, wherein TAA 1 is CD133.
236. The MBM of embodiment 235, wherein TAA 2 is FLT3.
237. The MBM of embodiment 235, wherein TAA 2 is CD52.
238. The MBM of embodiment 235, wherein TAA 2 is TNFRSF13C.
239. The MBM of embodiment 235, wherein TAA 2 is TNFRSF13B.
240. The MBM of embodiment 235, wherein TAA 2 is CXCR4.
241. The MBM of embodiment 235, wherein TAA 2 is PD-L1.
242. The MBM of embodiment 235, wherein TAA 2 is LY9.
243. The MBM of embodiment 235, wherein TAA 2 is CD200.
244. The MBM of embodiment 235, wherein TAA 2 is FCGR2B.
245. The MBM of embodiment 235, wherein TAA 2 is CD21.
246. The MBM of embodiment 235, wherein TAA 2 is CD23.
247. The MBM of embodiment 235, wherein TAA 2 is CD24.
248. The MBM of embodiment 235, wherein TAA 2 is CD40L.
249. The MBM of embodiment 235, wherein TAA 2 is CD72.
250. The MBM of embodiment 235, wherein TAA 2 is CD79a.
251. The MBM of embodiment 235, wherein TAA 2 is CD79b.
252. The MBM of any one of embodiments 1 to 9, wherein TAA 1 is FLT3.
253. The MBM of embodiment 252, wherein TAA 2 is CD52.
254. The MBM of embodiment 252, wherein TAA 2 is TNFRSF13C.
255. The MBM of embodiment 252, wherein TAA 2 is TNFRSF13B.
256. The MBM of embodiment 252, wherein TAA 2 is CXCR4.
257. The MBM of embodiment 252, wherein TAA 2 is PD-L1.
258. The MBM of embodiment 252, wherein TAA 2 is LY9.
259. The MBM of embodiment 252, wherein TAA 2 is CD200.
260. The MBM of embodiment 252, wherein TAA 2 is FCGR2B.
261. The MBM of embodiment 252, wherein TAA 2 is CD21.
262. The MBM of embodiment 252, wherein TAA 2 is CD23.
263. The MBM of embodiment 252, wherein TAA 2 is CD24.
264. The MBM of embodiment 252, wherein TAA 2 is CD40L.
265. The MBM of embodiment 252, wherein TAA 2 is CD72.
266. The MBM of embodiment 252, wherein TAA 2 is CD79a.
267. The MBM of embodiment 252, wherein TAA 2 is CD79b.
268. The MBM of any one of embodiments 1 to 9, wherein TAA 1 is CD52.
269. The MBM of embodiment 268, wherein TAA 2 is TNFRSF13C.
270. The MBM of embodiment 268, wherein TAA 2 is TNFRSF13B.
271. The MBM of embodiment 268, wherein TAA 2 is CXCR4.
272. The MBM of embodiment 268, wherein TAA 2 is PD-L1.
273. The MBM of embodiment 268, wherein TAA 2 is LY9.
274. The MBM of embodiment 268, wherein TAA 2 is CD200.
275. The MBM of embodiment 268, wherein TAA 2 is FCGR2B.
276. The MBM of embodiment 268, wherein TAA 2 is CD21.
277. The MBM of embodiment 268, wherein TAA 2 is CD23.
278. The MBM of embodiment 268, wherein TAA 2 is CD24.
279. The MBM of embodiment 268, wherein TAA 2 is CD40L.
280. The MBM of embodiment 268, wherein TAA 2 is CD72.
281. The MBM of embodiment 268, wherein TAA 2 is CD79a.
282. The MBM of embodiment 268, wherein TAA 2 is CD79b.
283. The MBM of any one of embodiments 1 to 9, wherein TAA 1 is TNFRSF13C.
284. The MBM of embodiment 283, wherein TAA 2 is TNFRSF13B.
285. The MBM of embodiment 283, wherein TAA 2 is CXCR4.
286. The MBM of embodiment 283, wherein TAA 2 is PD-L1.
287. The MBM of embodiment 283, wherein TAA 2 is LY9.
288. The MBM of embodiment 283, wherein TAA 2 is CD200.
289. The MBM of embodiment 283, wherein TAA 2 is FCGR2B.
290. The MBM of embodiment 283, wherein TAA 2 is CD21.
291. The MBM of embodiment 283, wherein TAA 2 is CD23.
292. The MBM of embodiment 283, wherein TAA 2 is CD24.
293. The MBM of embodiment 283, wherein TAA 2 is CD40L.
294. The MBM of embodiment 283, wherein TAA 2 is CD72.
295. The MBM of embodiment 283, wherein TAA 2 is CD79a.
296. The MBM of embodiment 283, wherein TAA 2 is CD79b.
297. The MBM of any one of embodiments 1 to 9, wherein TAA 1 is CXCR4.
298. The MBM of embodiment 297, wherein TAA 2 is PD-L1.
299. The MBM of embodiment 297, wherein TAA 2 is LY9.
300. The MBM of embodiment 297, wherein TAA 2 is CD200.
301. The MBM of embodiment 297, wherein TAA 2 is FCGR2B.
302. The MBM of embodiment 297, wherein TAA 2 is CD21.
303. The MBM of embodiment 297, wherein TAA 2 is CD23.
304. The MBM of embodiment 297, wherein TAA 2 is CD24.
305. The MBM of embodiment 297, wherein TAA 2 is CD40L.
306. The MBM of embodiment 297, wherein TAA 2 is CD72.
307. The MBM of embodiment 297, wherein TAA 2 is CD79a.
308. The MBM of embodiment 297, wherein TAA 2 is CD79b.
309. The MBM of any one of embodiments 1 to 9, wherein TAA 1 is PD-L1.
310. The MBM of embodiment 309, wherein TAA 2 is LY9.
311. The MBM of embodiment 309, wherein TAA 2 is CD200.
312. The MBM of embodiment 309, wherein TAA 2 is FCGR2B.
313. The MBM of embodiment 309, wherein TAA 2 is CD21.
314. The MBM of embodiment 309, wherein TAA 2 is CD23.
315. The MBM of embodiment 309, wherein TAA 2 is CD24.
316. The MBM of embodiment 309, wherein TAA 2 is CD40L.
317. The MBM of embodiment 309, wherein TAA 2 is CD72.
318. The MBM of embodiment 309, wherein TAA 2 is CD79a.
319. The MBM of embodiment 309, wherein TAA 2 is CD79b.
320. The MBM of any one of embodiments 1 to 9, wherein TAA 1 is LY9.
321. The MBM of embodiment 320, wherein TAA 2 is CD200.
322. The MBM of embodiment 320, wherein TAA 2 is FCGR2B.
323. The MBM of embodiment 320, wherein TAA 2 is CD21.
324. The MBM of embodiment 320, wherein TAA 2 is CD23.
325. The MBM of embodiment 320, wherein TAA 2 is CD24.
326. The MBM of embodiment 320, wherein TAA 2 is CD40L.
327. The MBM of embodiment 320, wherein TAA 2 is CD72.
328. The MBM of embodiment 320, wherein TAA 2 is CD79a.
329. The MBM of embodiment 320, wherein TAA 2 is CD79b.
330. The MBM of any one of embodiments 1 to 9, wherein TAA 1 is CD200.
331. The MBM of embodiment 330, wherein TAA 2 is FCGR2B.
332. The MBM of embodiment 330, wherein TAA 2 is CD21.
333. The MBM of embodiment 330, wherein TAA 2 is CD23.
334. The MBM of embodiment 330, wherein TAA 2 is CD24.
335. The MBM of embodiment 330, wherein TAA 2 is CD40L.
336. The MBM of embodiment 330, wherein TAA 2 is CD72.
337. The MBM of embodiment 330, wherein TAA 2 is CD79a.
338. The MBM of embodiment 330, wherein TAA 2 is CD79b.
339. The MBM of any one of embodiments 1 to 9, wherein TAA 1 is FCGR2B.
340. The MBM of embodiment 339, wherein TAA 2 is CD21.
341. The MBM of embodiment 339, wherein TAA 2 is CD23.
342. The MBM of embodiment 339, wherein TAA 2 is CD24.
343. The MBM of embodiment 339, wherein TAA 2 is CD40L.
344. The MBM of embodiment 339, wherein TAA 2 is CD72.
345. The MBM of embodiment 339, wherein TAA 2 is CD79a.
346. The MBM of embodiment 339, wherein TAA 2 is CD79b.
347. The MBM of any one of embodiments 1 to 9, wherein TAA 1 is CD21.
348. The MBM of embodiment 347, wherein TAA 2 is CD23.
349. The MBM of embodiment 347, wherein TAA 2 is CD24.
350. The MBM of embodiment 347, wherein TAA 2 is CD40L.
351. The MBM of embodiment 347, wherein TAA 2 is CD72.
352. The MBM of embodiment 347, wherein TAA 2 is CD79a.
353. The MBM of embodiment 347, wherein TAA 2 is CD79b.
354. The MBM of any one of embodiments 1 to 9, wherein TAA 1 is CD23.
355. The MBM of embodiment 354, wherein TAA 2 is CD24.
356. The MBM of embodiment 354, wherein TAA 2 is CD40L.
357. The MBM of embodiment 354, wherein TAA 2 is CD72.
358. The MBM of embodiment 354, wherein TAA 2 is CD79a.
359. The MBM of embodiment 354, wherein TAA 2 is CD79b.
360. The MBM of any one of embodiments 1 to 9, wherein TAA 1 is CD24.
361. The MBM of embodiment 360, wherein TAA 2 is CD40L.
362. The MBM of embodiment 360, wherein TAA 2 is CD72.
363. The MBM of embodiment 360, wherein TAA 2 is CD79a.
364. The MBM of embodiment 360, wherein TAA 2 is CD79b.
365. The MBM of any one of embodiments 1 to 9, wherein TAA 1 is CD40L.
366. The MBM of embodiment 365, wherein TAA 2 is CD72.
367. The MBM of embodiment 365, wherein TAA 2 is CD79a.
368. The MBM of embodiment 365, wherein TAA 2 is CD79b.
369. The MBM of any one of embodiments 1 to 9, wherein TAA 1 is CD72.
370. The MBM of embodiment 369, wherein TAA 2 is CD79a.
371. The MBM of embodiment 369, wherein TAA 2 is CD79b.
372. The MBM of any one of embodiments 1 to 9, wherein TAA 1 is CD79a.
373. The MBM of embodiment 372, wherein TAA 2 is CD79b.
374. The MBM of any one of embodiments 1 to 9, wherein TAA 2 is CD19.
375. The MBM of embodiment 374, wherein TAA 1 is CD20.
376. The MBM of embodiment 374, wherein TAA 1 is CD22.
377. The MBM of embodiment 374, wherein TAA 1 is CD123.
378. The MBM of embodiment 374, wherein TAA 1 is BCMA.
379. The MBM of embodiment 374, wherein TAA 1 is CD33.
380. The MBM of embodiment 374, wherein TAA 1 is CLL1.
381. The MBM of embodiment 374, wherein TAA 1 is CD138.
382. The MBM of embodiment 374, wherein TAA 1 is CS1.
383. The MBM of embodiment 374, wherein TAA 1 is CD38.
384. The MBM of embodiment 374, wherein TAA 1 is CD133.
385. The MBM of embodiment 374, wherein TAA 1 is FLT3.
386. The MBM of embodiment 374, wherein TAA 1 is CD52.
387. The MBM of embodiment 374, wherein TAA 1 is TNFRSF13C.
388. The MBM of embodiment 374, wherein TAA 1 is TNFRSF13B.
389. The MBM of embodiment 374, wherein TAA 1 is CXCR4.
390. The MBM of embodiment 374, wherein TAA 1 is PD-L1.
391. The MBM of embodiment 374, wherein TAA 1 is LY9.
392. The MBM of embodiment 374, wherein TAA 1 is CD200.
393. The MBM of embodiment 374, wherein TAA 1 is FCGR2B.
394. The MBM of embodiment 374, wherein TAA 1 is CD21.
395. The MBM of embodiment 374, wherein TAA 1 is CD23.
396. The MBM of embodiment 374, wherein TAA 1 is CD24.
397. The MBM of embodiment 374, wherein TAA 1 is CD40L.
398. The MBM of embodiment 374, wherein TAA 1 is CD72.
399. The MBM of embodiment 374, wherein TAA 1 is CD79a.
400. The MBM of embodiment 374, wherein TAA 1 is CD79b.
401. The MBM of any one of embodiments 1 to 9, wherein TAA 2 is CD20.
402. The MBM of embodiment 401, wherein TAA 1 is CD22.
403. The MBM of embodiment 401, wherein TAA 1 is CD123.
404. The MBM of embodiment 401, wherein TAA 1 is BCMA.
405. The MBM of embodiment 401, wherein TAA 1 is CD33.
406. The MBM of embodiment 401, wherein TAA 1 is CLL1.
407. The MBM of embodiment 401, wherein TAA 1 is CD138.
408. The MBM of embodiment 401, wherein TAA 1 is CS1.
409. The MBM of embodiment 401, wherein TAA 1 is CD38.
410. The MBM of embodiment 401, wherein TAA 1 is CD133.
411. The MBM of embodiment 401, wherein TAA 1 is FLT3.
412. The MBM of embodiment 401, wherein TAA 1 is CD52.
413. The MBM of embodiment 401, wherein TAA 1 is TNFRSF13C.
414. The MBM of embodiment 401, wherein TAA 1 is TNFRSF13B.
415. The MBM of embodiment 401, wherein TAA 1 is CXCR4.
416. The MBM of embodiment 401, wherein TAA 1 is PD-L1.
417. The MBM of embodiment 401, wherein TAA 1 is LY9.
418. The MBM of embodiment 401, wherein TAA 1 is CD200.
419. The MBM of embodiment 401, wherein TAA 1 is FCGR2B.
420. The MBM of embodiment 401, wherein TAA 1 is CD21.
421. The MBM of embodiment 401, wherein TAA 1 is CD23.
422. The MBM of embodiment 401, wherein TAA 1 is CD24.
423. The MBM of embodiment 401, wherein TAA 1 is CD40L.
424. The MBM of embodiment 401, wherein TAA 1 is CD72.
425. The MBM of embodiment 401, wherein TAA 1 is CD79a.
426. The MBM of embodiment 401, wherein TAA 1 is CD79b.
427. The MBM of any one of embodiments 1 to 9, wherein TAA 2 is CD22.
428. The MBM of embodiment 427, wherein TAA 1 is CD123.
429. The MBM of embodiment 427, wherein TAA 1 is BCMA.
430. The MBM of embodiment 427, wherein TAA 1 is CD33.
431. The MBM of embodiment 427, wherein TAA 1 is CLL1.
432. The MBM of embodiment 427, wherein TAA 1 is CD138.
433. The MBM of embodiment 427, wherein TAA 1 is CS1.
434. The MBM of embodiment 427, wherein TAA 1 is CD38.
435. The MBM of embodiment 427, wherein TAA 1 is CD133.
436. The MBM of embodiment 427, wherein TAA 1 is FLT3.
437. The MBM of embodiment 427, wherein TAA 1 is CD52.
438. The MBM of embodiment 427, wherein TAA 1 is TNFRSF13C.
439. The MBM of embodiment 427, wherein TAA 1 is TNFRSF13B.
440. The MBM of embodiment 427, wherein TAA 1 is CXCR4.
441. The MBM of embodiment 427, wherein TAA 1 is PD-L1.
442. The MBM of embodiment 427, wherein TAA 1 is LY9.
443. The MBM of embodiment 427, wherein TAA 1 is CD200.
444. The MBM of embodiment 427, wherein TAA 1 is FCGR2B.
445. The MBM of embodiment 427, wherein TAA 1 is CD21.
446. The MBM of embodiment 427, wherein TAA 1 is CD23.
447. The MBM of embodiment 427, wherein TAA 1 is CD24.
448. The MBM of embodiment 427, wherein TAA 1 is CD40L.
449. The MBM of embodiment 427, wherein TAA 1 is CD72.
450. The MBM of embodiment 427, wherein TAA 1 is CD79a.
451. The MBM of embodiment 427, wherein TAA 1 is CD79b.
452. The MBM of any one of embodiments 1 to 9, wherein TAA 2 is CD123.
453. The MBM of embodiment 452, wherein TAA 1 is BCMA.
454. The MBM of embodiment 452, wherein TAA 1 is CD33.
455. The MBM of embodiment 452, wherein TAA 1 is CLL1.
456. The MBM of embodiment 452, wherein TAA 1 is CD138.
457. The MBM of embodiment 452, wherein TAA 1 is CS1.
458. The MBM of embodiment 452, wherein TAA 1 is CD38.
459. The MBM of embodiment 452, wherein TAA 1 is CD133.
460. The MBM of embodiment 452, wherein TAA 1 is FLT3.
461. The MBM of embodiment 452, wherein TAA 1 is CD52.
462. The MBM of embodiment 452, wherein TAA 1 is TNFRSF13C.
463. The MBM of embodiment 452, wherein TAA 1 is TNFRSF13B.
464. The MBM of embodiment 452, wherein TAA 1 is CXCR4.
465. The MBM of embodiment 452, wherein TAA 1 is PD-L1.
466. The MBM of embodiment 452, wherein TAA 1 is LY9.
467. The MBM of embodiment 452, wherein TAA 1 is CD200.
468. The MBM of embodiment 452, wherein TAA 1 is FCGR2B.
469. The MBM of embodiment 452, wherein TAA 1 is CD21.
470. The MBM of embodiment 452, wherein TAA 1 is CD23.
471. The MBM of embodiment 452, wherein TAA 1 is CD24.
472. The MBM of embodiment 452, wherein TAA 1 is CD40L.
473. The MBM of embodiment 452, wherein TAA 1 is CD72.
474. The MBM of embodiment 452, wherein TAA 1 is CD79a.
475. The MBM of embodiment 452, wherein TAA 1 is CD79b.
476. The MBM of any one of embodiments 1 to 9, wherein TAA 2 is BCMA.
477. The MBM of embodiment 476, wherein TAA 1 is CD33.
478. The MBM of embodiment 476, wherein TAA 1 is CLL1.
479. The MBM of embodiment 476, wherein TAA 1 is CD138.
480. The MBM of embodiment 476, wherein TAA 1 is CS1.
481. The MBM of embodiment 476, wherein TAA 1 is CD38.
482. The MBM of embodiment 476, wherein TAA 1 is CD133.
483. The MBM of embodiment 476, wherein TAA 1 is FLT3.
484. The MBM of embodiment 476, wherein TAA 1 is CD52.
485. The MBM of embodiment 476, wherein TAA 1 is TNFRSF13C.
486. The MBM of embodiment 476, wherein TAA 1 is TNFRSF13B.
487. The MBM of embodiment 476, wherein TAA 1 is CXCR4.
488. The MBM of embodiment 476, wherein TAA 1 is PD-L1.
489. The MBM of embodiment 476, wherein TAA 1 is LY9.
490. The MBM of embodiment 476, wherein TAA 1 is CD200.
491. The MBM of embodiment 476, wherein TAA 1 is FCGR2B.
492. The MBM of embodiment 476, wherein TAA 1 is CD21.
493. The MBM of embodiment 476, wherein TAA 1 is CD23.
494. The MBM of embodiment 476, wherein TAA 1 is CD24.
495. The MBM of embodiment 476, wherein TAA 1 is CD40L.
496. The MBM of embodiment 476, wherein TAA 1 is CD72.
497. The MBM of embodiment 476, wherein TAA 1 is CD79a.
498. The MBM of embodiment 476, wherein TAA 1 is CD79b.
499. The MBM of any one of embodiments 1 to 9, wherein TAA 2 is CD33.
500. The MBM of embodiment 499, wherein TAA 1 is CLL1.
501. The MBM of embodiment 499, wherein TAA 1 is CD138.
502. The MBM of embodiment 499, wherein TAA 1 is CS1.
503. The MBM of embodiment 499, wherein TAA 1 is CD38.
504. The MBM of embodiment 499, wherein TAA 1 is CD133.
505. The MBM of embodiment 499, wherein TAA 1 is FLT3.
506. The MBM of embodiment 499, wherein TAA 1 is CD52.
507. The MBM of embodiment 499, wherein TAA 1 is TNFRSF13C.
508. The MBM of embodiment 499, wherein TAA 1 is TNFRSF13B.
509. The MBM of embodiment 499, wherein TAA 1 is CXCR4.
510. The MBM of embodiment 499, wherein TAA 1 is PD-L1.
511. The MBM of embodiment 499, wherein TAA 1 is LY9.
512. The MBM of embodiment 499, wherein TAA 1 is CD200.
513. The MBM of embodiment 499, wherein TAA 1 is FCGR2B.
514. The MBM of embodiment 499, wherein TAA 1 is CD21.
515. The MBM of embodiment 499, wherein TAA 1 is CD23.
516. The MBM of embodiment 499, wherein TAA 1 is CD24.
517. The MBM of embodiment 499, wherein TAA 1 is CD40L.
518. The MBM of embodiment 499, wherein TAA 1 is CD72.
519. The MBM of embodiment 499, wherein TAA 1 is CD79a.
520. The MBM of embodiment 499, wherein TAA 1 is CD79b.
521. The MBM of any one of embodiments 1 to 9, wherein TAA 2 is CLL1.
522. The MBM of embodiment 521, wherein TAA 1 is CD138.
523. The MBM of embodiment 521, wherein TAA 1 is CS1.
524. The MBM of embodiment 521, wherein TAA 1 is CD38.
525. The MBM of embodiment 521, wherein TAA 1 is CD133.
526. The MBM of embodiment 521, wherein TAA 1 is FLT3.
527. The MBM of embodiment 521, wherein TAA 1 is CD52.
528. The MBM of embodiment 521, wherein TAA 1 is TNFRSF13C.
529. The MBM of embodiment 521, wherein TAA 1 is TNFRSF13B.
530. The MBM of embodiment 521, wherein TAA 1 is CXCR4.
531. The MBM of embodiment 521, wherein TAA 1 is PD-L1.
532. The MBM of embodiment 521, wherein TAA 1 is LY9.
533. The MBM of embodiment 521, wherein TAA 1 is CD200.
534. The MBM of embodiment 521, wherein TAA 1 is FCGR2B.
535. The MBM of embodiment 521, wherein TAA 1 is CD21.
536. The MBM of embodiment 521, wherein TAA 1 is CD23.
537. The MBM of embodiment 521, wherein TAA 1 is CD24.
538. The MBM of embodiment 521, wherein TAA 1 is CD40L.
539. The MBM of embodiment 521, wherein TAA 1 is CD72.
540. The MBM of embodiment 521, wherein TAA 1 is CD79a.
541. The MBM of embodiment 521, wherein TAA 1 is CD79b.
542. The MBM of any one of embodiments 1 to 9, wherein TAA 2 is CD138.
543. The MBM of embodiment 542, wherein TAA 1 is CS1.
544. The MBM of embodiment 542, wherein TAA 1 is CD38.
545. The MBM of embodiment 542, wherein TAA 1 is CD133.
546. The MBM of embodiment 542, wherein TAA 1 is FLT3.
547. The MBM of embodiment 542, wherein TAA 1 is CD52.
548. The MBM of embodiment 542, wherein TAA 1 is TNFRSF13C.
549. The MBM of embodiment 542, wherein TAA 1 is TNFRSF13B.
550. The MBM of embodiment 542, wherein TAA 1 is CXCR4.
551. The MBM of embodiment 542, wherein TAA 1 is PD-L1.
552. The MBM of embodiment 542, wherein TAA 1 is LY9.
553. The MBM of embodiment 542, wherein TAA 1 is CD200.
554. The MBM of embodiment 542, wherein TAA 1 is FCGR2B.
555. The MBM of embodiment 542, wherein TAA 1 is CD21.
556. The MBM of embodiment 542, wherein TAA 1 is CD23.
557. The MBM of embodiment 542, wherein TAA 1 is CD24.
558. The MBM of embodiment 542, wherein TAA 1 is CD40L.
559. The MBM of embodiment 542, wherein TAA 1 is CD72.
560. The MBM of embodiment 542, wherein TAA 1 is CD79a.
561. The MBM of embodiment 542, wherein TAA 1 is CD79b.
562. The MBM of any one of embodiments 1 to 9, wherein TAA 2 is CS1.
563. The MBM of embodiment 562, wherein TAA 1 is CD38.
564. The MBM of embodiment 562, wherein TAA 1 is CD133.
565. The MBM of embodiment 562, wherein TAA 1 is FLT3.
566. The MBM of embodiment 562, wherein TAA 1 is CD52.
567. The MBM of embodiment 562, wherein TAA 1 is TNFRSF13C.
568. The MBM of embodiment 562, wherein TAA 1 is TNFRSF13B.
569. The MBM of embodiment 562, wherein TAA 1 is CXCR4.
570. The MBM of embodiment 562, wherein TAA 1 is PD-L1.
571. The MBM of embodiment 562, wherein TAA 1 is LY9.
572. The MBM of embodiment 562, wherein TAA 1 is CD200.
573. The MBM of embodiment 562, wherein TAA 1 is FCGR2B.
574. The MBM of embodiment 562, wherein TAA 1 is CD21.
575. The MBM of embodiment 562, wherein TAA 1 is CD23.
576. The MBM of embodiment 562, wherein TAA 1 is CD24.
577. The MBM of embodiment 562, wherein TAA 1 is CD40L.
578. The MBM of embodiment 562, wherein TAA 1 is CD72.
579. The MBM of embodiment 562, wherein TAA 1 is CD79a.
580. The MBM of embodiment 562, wherein TAA 1 is CD79b.
581. The MBM of any one of embodiments 1 to 9, wherein TAA 2 is CD38.
582. The MBM of embodiment 581, wherein TAA 1 is CD133.
583. The MBM of embodiment 581, wherein TAA 1 is FLT3.
584. The MBM of embodiment 581, wherein TAA 1 is CD52.
585. The MBM of embodiment 581, wherein TAA 1 is TNFRSF13C.
586. The MBM of embodiment 581, wherein TAA 1 is TNFRSF13B.
587. The MBM of embodiment 581, wherein TAA 1 is CXCR4.
588. The MBM of embodiment 581, wherein TAA 1 is PD-L1.
589. The MBM of embodiment 581, wherein TAA 1 is LY9.
590. The MBM of embodiment 581, wherein TAA 1 is CD200.
591. The MBM of embodiment 581, wherein TAA 1 is FCGR2B.
592. The MBM of embodiment 581, wherein TAA 1 is CD21.
593. The MBM of embodiment 581, wherein TAA 1 is CD23.
594. The MBM of embodiment 581, wherein TAA 1 is CD24.
595. The MBM of embodiment 581, wherein TAA 1 is CD40L.
596. The MBM of embodiment 581, wherein TAA 1 is CD72.
597. The MBM of embodiment 581, wherein TAA 1 is CD79a.
598. The MBM of embodiment 581, wherein TAA 1 is CD79b.
599. The MBM of any one of embodiments 1 to 9, wherein TAA 2 is CD133.
600. The MBM of embodiment 599, wherein TAA 1 is FLT3.
601. The MBM of embodiment 599, wherein TAA 1 is CD52.
602. The MBM of embodiment 599, wherein TAA 1 is TNFRSF13C.
603. The MBM of embodiment 599, wherein TAA 1 is TNFRSF13B.
604. The MBM of embodiment 599, wherein TAA 1 is CXCR4.
605. The MBM of embodiment 599, wherein TAA 1 is PD-L1.
606. The MBM of embodiment 599, wherein TAA 1 is LY9.
607. The MBM of embodiment 599, wherein TAA 1 is CD200.
608. The MBM of embodiment 599, wherein TAA 1 is FCGR2B.
609. The MBM of embodiment 599, wherein TAA 1 is CD21.
610. The MBM of embodiment 599, wherein TAA 1 is CD23.
611. The MBM of embodiment 599, wherein TAA 1 is CD24.
612. The MBM of embodiment 599, wherein TAA 1 is CD40L.
613. The MBM of embodiment 599, wherein TAA 1 is CD72.
614. The MBM of embodiment 599, wherein TAA 1 is CD79a.
615. The MBM of embodiment 599, wherein TAA 1 is CD79b.
616. The MBM of any one of embodiments 1 to 9, wherein TAA 2 is FLT3.
617. The MBM of embodiment 616, wherein TAA 1 is CD52.
618. The MBM of embodiment 616, wherein TAA 1 is TNFRSF13C.
619. The MBM of embodiment 616, wherein TAA 1 is TNFRSF13B.
620. The MBM of embodiment 616, wherein TAA 1 is CXCR4.
621. The MBM of embodiment 616, wherein TAA 1 is PD-L1.
622. The MBM of embodiment 616, wherein TAA 1 is LY9.
623. The MBM of embodiment 616, wherein TAA 1 is CD200.
624. The MBM of embodiment 616, wherein TAA 1 is FCGR2B.
625. The MBM of embodiment 616, wherein TAA 1 is CD21.
626. The MBM of embodiment 616, wherein TAA 1 is CD23.
627. The MBM of embodiment 616, wherein TAA 1 is CD24.
628. The MBM of embodiment 616, wherein TAA 1 is CD40L.
629. The MBM of embodiment 616, wherein TAA 1 is CD72.
630. The MBM of embodiment 616, wherein TAA 1 is CD79a.
631. The MBM of embodiment 616, wherein TAA 1 is CD79b.
632. The MBM of any one of embodiments 1 to 9, wherein TAA 2 is CD52.
633. The MBM of embodiment 632, wherein TAA 1 is TNFRSF13C.
634. The MBM of embodiment 632, wherein TAA 1 is TNFRSF13B.
635. The MBM of embodiment 632, wherein TAA 1 is CXCR4.
636. The MBM of embodiment 632, wherein TAA 1 is PD-L1.
637. The MBM of embodiment 632, wherein TAA 1 is LY9.
638. The MBM of embodiment 632, wherein TAA 1 is CD200.
639. The MBM of embodiment 632, wherein TAA 1 is FCGR2B.
640. The MBM of embodiment 632, wherein TAA 1 is CD21.
641. The MBM of embodiment 632, wherein TAA 1 is CD23.
642. The MBM of embodiment 632, wherein TAA 1 is CD24.
643. The MBM of embodiment 632, wherein TAA 1 is CD40L.
644. The MBM of embodiment 632, wherein TAA 1 is CD72.
645. The MBM of embodiment 632, wherein TAA 1 is CD79a.
646. The MBM of embodiment 632, wherein TAA 1 is CD79b.
647. The MBM of any one of embodiments 1 to 9, wherein TAA 2 is TNFRSF13C.
648. The MBM of embodiment 647, wherein TAA 1 is TNFRSF13B.
649. The MBM of embodiment 647, wherein TAA 1 is CXCR4.
650. The MBM of embodiment 647, wherein TAA 1 is PD-L1.
651. The MBM of embodiment 647, wherein TAA 1 is LY9.
652. The MBM of embodiment 647, wherein TAA 1 is CD200.
653. The MBM of embodiment 647, wherein TAA 1 is FCGR2B.
654. The MBM of embodiment 647, wherein TAA 1 is CD21.
655. The MBM of embodiment 647, wherein TAA 1 is CD23.
656. The MBM of embodiment 647, wherein TAA 1 is CD24.
657. The MBM of embodiment 647, wherein TAA 1 is CD40L.
658. The MBM of embodiment 647, wherein TAA 1 is CD72.
659. The MBM of embodiment 647, wherein TAA 1 is CD79a.
660. The MBM of embodiment 647, wherein TAA 1 is CD79b.
661. The MBM of any one of embodiments 1 to 9, wherein TAA 2 is CXCR4.
662. The MBM of embodiment 661, wherein TAA 1 is PD-L1.
663. The MBM of embodiment 661, wherein TAA 1 is LY9.
664. The MBM of embodiment 661, wherein TAA 1 is CD200.
665. The MBM of embodiment 661, wherein TAA 1 is FCGR2B.
666. The MBM of embodiment 661, wherein TAA 1 is CD21.
667. The MBM of embodiment 661, wherein TAA 1 is CD23.
668. The MBM of embodiment 661, wherein TAA 1 is CD24.
669. The MBM of embodiment 661, wherein TAA 1 is CD40L.
670. The MBM of embodiment 661, wherein TAA 1 is CD72.
671. The MBM of embodiment 661, wherein TAA 1 is CD79a.
672. The MBM of embodiment 661, wherein TAA 1 is CD79b.
673. The MBM of any one of embodiments 1 to 9, wherein TAA 2 is PD-L1.
674. The MBM of embodiment 673, wherein TAA 1 is LY9.
675. The MBM of embodiment 673, wherein TAA 1 is CD200.
676. The MBM of embodiment 673, wherein TAA 1 is FCGR2B.
677. The MBM of embodiment 673, wherein TAA 1 is CD21.
678. The MBM of embodiment 673, wherein TAA 1 is CD23.
679. The MBM of embodiment 673, wherein TAA 1 is CD24.
680. The MBM of embodiment 673, wherein TAA 1 is CD40L.
681. The MBM of embodiment 673, wherein TAA 1 is CD72.
682. The MBM of embodiment 673, wherein TAA 1 is CD79a.
683. The MBM of embodiment 673, wherein TAA 1 is CD79b.
684. The MBM of any one of embodiments 1 to 9, wherein TAA 2 is LY9.
685. The MBM of embodiment 684, wherein TAA 1 is CD200.
686. The MBM of embodiment 684, wherein TAA 1 is FCGR2B.
687. The MBM of embodiment 684, wherein TAA 1 is CD21.
688. The MBM of embodiment 684, wherein TAA 1 is CD23.
689. The MBM of embodiment 684, wherein TAA 1 is CD24.
690. The MBM of embodiment 684, wherein TAA 1 is CD40L.
691. The MBM of embodiment 684, wherein TAA 1 is CD72.
692. The MBM of embodiment 684, wherein TAA 1 is CD79a.
693. The MBM of embodiment 684, wherein TAA 1 is CD79b.
694. The MBM of any one of embodiments 1 to 9, wherein TAA 2 is CD200.
695. The MBM of embodiment 694, wherein TAA 1 is FCGR2B.
696. The MBM of embodiment 694, wherein TAA 1 is CD21.
697. The MBM of embodiment 694, wherein TAA 1 is CD23.
698. The MBM of embodiment 694, wherein TAA 1 is CD24.
699. The MBM of embodiment 694, wherein TAA 1 is CD40L.
700. The MBM of embodiment 694, wherein TAA 1 is CD72.
701. The MBM of embodiment 694, wherein TAA 1 is CD79a.
702. The MBM of embodiment 694, wherein TAA 1 is CD79b.
703. The MBM of any one of embodiments 1 to 9, wherein TAA 2 is FCGR2B.
704. The MBM of embodiment 703, wherein TAA 1 is CD21.
705. The MBM of embodiment 703, wherein TAA 1 is CD23.
706. The MBM of embodiment 703, wherein TAA 1 is CD24.
707. The MBM of embodiment 703, wherein TAA 1 is CD40L.
708. The MBM of embodiment 703, wherein TAA 1 is CD72.
709. The MBM of embodiment 703, wherein TAA 1 is CD79a.
710. The MBM of embodiment 703, wherein TAA 1 is CD79b.
711. The MBM of any one of embodiments 1 to 9, wherein TAA 2 is CD21.
712. The MBM of embodiment 711, wherein TAA 1 is CD23.
713. The MBM of embodiment 711, wherein TAA 1 is CD24.
714. The MBM of embodiment 711, wherein TAA 1 is CD40L.
715. The MBM of embodiment 711, wherein TAA 1 is CD72.
716. The MBM of embodiment 711, wherein TAA 1 is CD79a.
717. The MBM of embodiment 711, wherein TAA 1 is CD79b.
718. The MBM of any one of embodiments 1 to 9, wherein TAA 2 is CD23.
719. The MBM of embodiment 718, wherein TAA 1 is CD24.
720. The MBM of embodiment 718, wherein TAA 1 is CD40L.
721. The MBM of embodiment 718, wherein TAA 1 is CD72.
722. The MBM of embodiment 718, wherein TAA 1 is CD79a.
723. The MBM of embodiment 718, wherein TAA 1 is CD79b.
724. The MBM of any one of embodiments 1 to 9, wherein TAA 2 is CD24.
725. The MBM of embodiment 724, wherein TAA 1 is CD40L.
726. The MBM of embodiment 724, wherein TAA 1 is CD72.
727. The MBM of embodiment 724, wherein TAA 1 is CD79a.
728. The MBM of embodiment 724, wherein TAA 1 is CD79b.
729. The MBM of any one of embodiments 1 to 9, wherein TAA 2 is CD40L.
730. The MBM of embodiment 729, wherein TAA 1 is CD72.
731. The MBM of embodiment 729, wherein TAA 1 is CD79a.
732. The MBM of embodiment 729, wherein TAA 1 is CD79b.
733. The MBM of any one of embodiments 1 to 9, wherein TAA 2 is CD72.
734. The MBM of embodiment 733, wherein TAA 1 is CD79a.
735. The MBM of embodiment 733, wherein TAA 1 is CD79b.
736. The MBM of any one of embodiments 1 to 9, wherein TAA 2 is CD79a.
737. The MBM of embodiment 736, wherein TAA 1 is CD79b.
738. The MBM of any one of embodiments 1 to 737, wherein ABM1 is an immunoglobulin scaffold-based ABM.
739. The MBM of embodiment 738, wherein ABM1 is an anti-TAA 1 antibody, an antibody fragment, an scFv, a dsFv, a Fv, a Fab, an scFab, a (Fab′)₂, a single domain antibody (SDAB), a VH or VL domain, or a camelid VHH domain.
740. The MBM of embodiment 739, wherein ABM1 is an scFv.
741. The MBM of embodiment 739, wherein ABM1 is a Fab.
742. The MBM of embodiment 741, wherein the Fab is a Fab heterodimer.
743. The MBM of any one of embodiments 738 to 742, wherein ABM1 comprises a binding sequence described in Table 10.
744. The MBM of embodiment 743, wherein ABM1 comprises the CDRs or variable region sequences of the antibodies set forth in Table 10.
745. The MBM of any one of embodiments 738 to 742, wherein:
   (a) if TAA 1 is BCMA, ABM1 optionally comprises a binding sequence described in Table 11; and
   (b) if TAA 1 is CD19, ABM1 optionally comprises a binding sequence described in Table 12.
746. The MBM of embodiment 745, wherein if TAA 1 is BCMA, ABM1 comprises the CDR sequences of BCMA-1.
747. The MBM of embodiment 745, wherein if TAA 1 is BCMA, ABM1 comprises the CDR sequences of BCMA-2.
748. The MBM of embodiment 745, wherein if TAA 1 is BCMA, ABM1 comprises the CDR sequences of BCMA-3.
749. The MBM of embodiment 745, wherein if TAA 1 is BCMA, ABM1 comprises the CDR sequences of BCMA-4.
750. The MBM of embodiment 745, wherein if TAA 1 is BCMA, ABM1 comprises the CDR sequences of BCMA-5.
751. The MBM of embodiment 745, wherein if TAA 1 is BCMA, ABM1 comprises the CDR sequences of BCMA-6.
752. The MBM of embodiment 745, wherein if TAA 1 is BCMA, ABM1 comprises the CDR sequences of BCMA-7.
753. The MBM of embodiment 745, wherein if TAA 1 is BCMA, ABM1 comprises the CDR sequences of BCMA-8.
754. The MBM of embodiment 745, wherein if TAA 1 is BCMA, ABM1 comprises the CDR sequences of BCMA-9.
755. The MBM of embodiment 745, wherein if TAA 1 is BCMA, ABM1 comprises the CDR sequences of BCMA-10.
756. The MBM of embodiment 745, wherein if TAA 1 is BCMA, ABM1 comprises the CDR sequences of BCMA-11.
757. The MBM of embodiment 745, wherein if TAA 1 is BCMA, ABM1 comprises the CDR sequences of BCMA-12.
758. The MBM of embodiment 745, wherein if TAA 1 is BCMA, ABM1 comprises the CDR sequences of BCMA-13.
759. The MBM of embodiment 745, wherein if TAA 1 is BCMA, ABM1 comprises the CDR sequences of BCMA-14.
760. The MBM of embodiment 745, wherein if TAA 1 is BCMA, ABM1 comprises the CDR sequences of BCMA-15.
761. The MBM of embodiment 745, wherein if TAA 1 is BCMA, ABM1 comprises the CDR sequences of BCMA-16.
762. The MBM of embodiment 745, wherein if TAA 1 is BCMA, ABM1 comprises the CDR sequences of BCMA-17.
763. The MBM of embodiment 745, wherein if TAA 1 is BCMA, ABM1 comprises the CDR sequences of BCMA-18.
764. The MBM of embodiment 745, wherein if TAA 1 is BCMA, ABM1 comprises the CDR sequences of BCMA-19.
765. The MBM of embodiment 745, wherein if TAA 1 is BCMA, ABM1 comprises the CDR sequences of BCMA-20.
766. The MBM of embodiment 745, wherein if TAA 1 is BCMA, ABM1 comprises the CDR sequences of BCMA-21.
767. The MBM of embodiment 745, wherein if TAA 1 is BCMA, ABM1 comprises the CDR sequences of BCMA-22.
768. The MBM of embodiment 745, wherein if TAA 1 is BCMA, ABM1 comprises the CDR sequences of BCMA-23.
769. The MBM of embodiment 745, wherein if TAA 1 is BCMA, ABM1 comprises the CDR sequences of BCMA-24.
770. The MBM of embodiment 745, wherein if TAA 1 is BCMA, ABM1 comprises the CDR sequences of BCMA-25.
771. The MBM of embodiment 745, wherein if TAA 1 is BCMA, ABM1 comprises the CDR sequences of BCMA-26.
772. The MBM of embodiment 745, wherein if TAA 1 is BCMA, ABM1 comprises the CDR sequences of BCMA-27.
773. The MBM of embodiment 745, wherein if TAA 1 is BCMA, ABM1 comprises the CDR sequences of BCMA-28.
774. The MBM of embodiment 745, wherein if TAA 1 is BCMA, ABM1 comprises the CDR sequences of BCMA-29.
775. The MBM of embodiment 745, wherein if TAA 1 is BCMA, ABM1 comprises the CDR sequences of BCMA-30.
776. The MBM of embodiment 745, wherein if TAA 1 is BCMA, ABM1 comprises the CDR sequences of BCMA-31.
777. The MBM of embodiment 745, wherein if TAA 1 is BCMA, ABM1 comprises the CDR sequences of BCMA-32.
778. The MBM of embodiment 745, wherein if TAA 1 is BCMA, ABM1 comprises the CDR sequences of BCMA-33.
779. The MBM of embodiment 745, wherein if TAA 1 is BCMA, ABM1 comprises the CDR sequences of BCMA-34.
780. The MBM of embodiment 745, wherein if TAA 1 is BCMA, ABM1 comprises the CDR sequences of BCMA-35.
781. The MBM of embodiment 745, wherein if TAA 1 is BCMA, ABM1 comprises the CDR sequences of BCMA-36.
782. The MBM of embodiment 745, wherein if TAA 1 is BCMA, ABM1 comprises the CDR sequences of BCMA-37.
783. The MBM of embodiment 745, wherein if TAA 1 is BCMA, ABM1 comprises the CDR sequences of BCMA-38.
784. The MBM of embodiment 745, wherein if TAA 1 is BCMA, ABM1 comprises the CDR sequences of BCMA-39.
785. The MBM of embodiment 745, wherein if TAA 1 is BCMA, ABM1 comprises the CDR sequences of BCMA-40.
786. The MBM of any one of embodiments 746 to 785, wherein the CDRs are defined by Kabat numbering, as set forth in Table 11B and 11E.
787. The MBM of any one of embodiments 746 to 785, wherein the CDRs are defined by Chothia numbering, as set forth in Table 11C and 11F.
788. MBM of any one of embodiments 746 to 785, wherein the CDRs are defined by a combination of Kabat and Chothia numbering, as set forth in Table 11D and 11G.
789. The MBM of embodiment 745, wherein if TAA 1 is BCMA, ABM1 comprises the heavy and light chain variable sequences of BCMA-1, as set forth in Table 11A.
790. The MBM of embodiment 745, wherein if TAA 1 is BCMA, ABM1 comprises the heavy and light chain variable sequences of BCMA-2, as set forth in Table 11A.
791. The MBM of embodiment 745, wherein if TAA 1 is BCMA, ABM1 comprises the heavy and light chain variable sequences of BCMA-3, as set forth in Table 11A.
792. The MBM of embodiment 745, wherein if TAA 1 is BCMA, ABM1 comprises the heavy and light chain variable sequences of BCMA-4, as set forth in Table 11A.
793. The MBM of embodiment 745, wherein if TAA 1 is BCMA, ABM1 comprises the heavy and light chain variable sequences of BCMA-5, as set forth in Table 11A.
794. The MBM of embodiment 745, wherein if TAA 1 is BCMA, ABM1 comprises the heavy and light chain variable sequences of BCMA-6, as set forth in Table 11A.
795. The MBM of embodiment 745, wherein if TAA 1 is BCMA, ABM1 comprises the heavy and light chain variable sequences of BCMA-7, as set forth in Table 11A.
796. The MBM of embodiment 745, wherein if TAA 1 is BCMA, ABM1 comprises the heavy and light chain variable sequences of BCMA-8, as set forth in Table 11A.
797. The MBM of embodiment 745, wherein if TAA 1 is BCMA, ABM1 comprises the heavy and light chain variable sequences of BCMA-9, as set forth in Table 11A.
798. The MBM of embodiment 745, wherein if TAA 1 is BCMA, ABM1 comprises the heavy and light chain variable sequences of BCMA-10, as set forth in Table 11A.
799. The MBM of embodiment 745, wherein if TAA 1 is BCMA, ABM1 comprises the heavy and light chain variable sequences of BCMA-11, as set forth in Table 11A.
800. The MBM of embodiment 745, wherein if TAA 1 is BCMA, ABM1 comprises the heavy and light chain variable sequences of BCMA-12, as set forth in Table 11A.
801. The MBM of embodiment 745, wherein if TAA 1 is BCMA, ABM1 comprises the heavy and light chain variable sequences of BCMA-13, as set forth in Table 11A.
802. The MBM of embodiment 745, wherein if TAA 1 is BCMA, ABM1 comprises the heavy and light chain variable sequences of BCMA-14, as set forth in Table 11A.
803. The MBM of embodiment 745, wherein if TAA 1 is BCMA, ABM1 comprises the heavy and light chain variable sequences of BCMA-15, as set forth in Table 11A.
804. The MBM of embodiment 745, wherein if TAA 1 is BCMA, ABM1 comprises the heavy and light chain variable sequences of BCMA-16, as set forth in Table 11A.
805. The MBM of embodiment 745, wherein if TAA 1 is BCMA, ABM1 comprises the heavy and light chain variable sequences of BCMA-17, as set forth in Table 11A.
806. The MBM of embodiment 745, wherein if TAA 1 is BCMA, ABM1 comprises the heavy and light chain variable sequences of BCMA-18, as set forth in Table 11A.
807. The MBM of embodiment 745, wherein if TAA 1 is BCMA, ABM1 comprises the heavy and light chain variable sequences of BCMA-19, as set forth in Table 11A.
808. The MBM of embodiment 745, wherein if TAA 1 is BCMA, ABM1 comprises the heavy and light chain variable sequences of BCMA-20, as set forth in Table 11A.
809. The MBM of embodiment 745, wherein if TAA 1 is BCMA, ABM1 comprises the heavy and light chain variable sequences of BCMA-21, as set forth in Table 11A.
810. The MBM of embodiment 745, wherein if TAA 1 is BCMA, ABM1 comprises the heavy and light chain variable sequences of BCMA-22, as set forth in Table 11A.
811. The MBM of embodiment 745, wherein if TAA 1 is BCMA, ABM1 comprises the heavy and light chain variable sequences of BCMA-23, as set forth in Table 11A.
812. The MBM of embodiment 745, wherein if TAA 1 is BCMA, ABM1 comprises the heavy and light chain variable sequences of BCMA-24, as set forth in Table 11A.
813. The MBM of embodiment 745, wherein if TAA 1 is BCMA, ABM1 comprises the heavy and light chain variable sequences of BCMA-25, as set forth in Table 11A.
814. The MBM of embodiment 745, wherein if TAA 1 is BCMA, ABM1 comprises the heavy and light chain variable sequences of BCMA-26, as set forth in Table 11A.
815. The MBM of embodiment 745, wherein if TAA 1 is BCMA, ABM1 comprises the heavy and light chain variable sequences of BCMA-27, as set forth in Table 11A.
816. The MBM of embodiment 745, wherein if TAA 1 is BCMA, ABM1 comprises the heavy and light chain variable sequences of BCMA-28, as set forth in Table 11A.
817. The MBM of embodiment 745, wherein if TAA 1 is BCMA, ABM1 comprises the heavy and light chain variable sequences of BCMA-29, as set forth in Table 11A.
818. The MBM of embodiment 745, wherein if TAA 1 is BCMA, ABM1 comprises the heavy and light chain variable sequences of BCMA-30, as set forth in Table 11A.
819. The MBM of embodiment 745, wherein if TAA 1 is BCMA, ABM1 comprises the heavy and light chain variable sequences of BCMA-31, as set forth in Table 11A.
820. The MBM of embodiment 745, wherein if TAA 1 is BCMA, ABM1 comprises the heavy and light chain variable sequences of BCMA-32, as set forth in Table 11A.
821. The MBM of embodiment 745, wherein if TAA 1 is BCMA, ABM1 comprises the heavy and light chain variable sequences of BCMA-33, as set forth in Table 11A.
822. The MBM of embodiment 745, wherein if TAA 1 is BCMA, ABM1 comprises the heavy and light chain variable sequences of BCMA-34, as set forth in Table 11A.
823. The MBM of embodiment 745, wherein if TAA 1 is BCMA, ABM1 comprises the heavy and light chain variable sequences of BCMA-35, as set forth in Table 11A.
824. The MBM of embodiment 745, wherein if TAA 1 is BCMA, ABM1 comprises the heavy and light chain variable sequences of BCMA-36, as set forth in Table 11A.
825. The MBM of embodiment 745, wherein if TAA 1 is BCMA, ABM1 comprises the heavy and light chain variable sequences of BCMA-37, as set forth in Table 11A.
826. The MBM of embodiment 745, wherein if TAA 1 is BCMA, ABM1 comprises the heavy and light chain variable sequences of BCMA-38, as set forth in Table 11A.
827. The MBM of embodiment 745, wherein if TAA 1 is BCMA, ABM1 comprises the heavy and light chain variable sequences of BCMA-39, as set forth in Table 11A.
828. The MBM of embodiment 745, wherein if TAA 1 is BCMA, ABM1 comprises the heavy and light chain variable sequences of BCMA-40, as set forth in Table 11A.
829. The MBM of embodiment 745, wherein if TAA 1 is BCMA, ABM1 comprises a scFv comprising the amino acid sequence of scFv corresponding to BCMA-1 as set forth in Table 11A.
830. The MBM of embodiment 745, wherein if TAA 1 is BCMA, ABM1 comprises a scFv comprising the amino acid sequence of scFv corresponding to BCMA-2 as set forth in Table 11A.
831. The MBM of embodiment 745, wherein if TAA 1 is BCMA, ABM1 comprises a scFv comprising the amino acid sequence of scFv corresponding to BCMA-3 as set forth in Table 11A.
832. The MBM of embodiment 745, wherein if TAA 1 is BCMA, ABM1 comprises a scFv comprising the amino acid sequence of scFv corresponding to BCMA-4 as set forth in Table 11A.
833. The MBM of embodiment 745, wherein if TAA 1 is BCMA, ABM1 comprises a scFv comprising the amino acid sequence of scFv corresponding to BCMA-5 as set forth in Table 11A.
834. The MBM of embodiment 745, wherein if TAA 1 is BCMA, ABM1 comprises a scFv comprising the amino acid sequence of scFv corresponding to BCMA-6 as set forth in Table 11A.
835. The MBM of embodiment 745, wherein if TAA 1 is BCMA, ABM1 comprises a scFv comprising the amino acid sequence of scFv corresponding to BCMA-7 as set forth in Table 11A.
836. The MBM of embodiment 745, wherein if TAA 1 is BCMA, ABM1 comprises a scFv comprising the amino acid sequence of scFv corresponding to BCMA-8 as set forth in Table 11A.
837. The MBM of embodiment 745, wherein if TAA 1 is BCMA, ABM1 comprises a scFv comprising the amino acid sequence of scFv corresponding to BCMA-9 as set forth in Table 11A.
838. The MBM of embodiment 745, wherein if TAA 1 is BCMA, ABM1 comprises a scFv comprising the amino acid sequence of scFv corresponding to BCMA-10 as set forth in Table 11A.
839. The MBM of embodiment 745, wherein if TAA 1 is BCMA, ABM1 comprises a scFv comprising the amino acid sequence of scFv corresponding to BCMA-11 as set forth in Table 11A.
840. The MBM of embodiment 745, wherein if TAA 1 is BCMA, ABM1 comprises a scFv comprising the amino acid sequence of scFv corresponding to BCMA-12 as set forth in Table 11A.
841. The MBM of embodiment 745, wherein if TAA 1 is BCMA, ABM1 comprises a scFv comprising the amino acid sequence of scFv corresponding to BCMA-13 as set forth in Table 11A.
842. The MBM of embodiment 745, wherein if TAA 1 is BCMA, ABM1 comprises a scFv comprising the amino acid sequence of scFv corresponding to BCMA-14 as set forth in Table 11A.
843. The MBM of embodiment 745, wherein if TAA 1 is BCMA, ABM1 comprises a scFv comprising the amino acid sequence of scFv corresponding to BCMA-15 as set forth in Table 11A.
844. The MBM of embodiment 745, wherein if TAA 1 is BCMA, ABM1 comprises a scFv comprising the amino acid sequence of scFv corresponding to BCMA-16 as set forth in Table 11A.
845. The MBM of embodiment 745, wherein if TAA 1 is BCMA, ABM1 comprises a scFv comprising the amino acid sequence of scFv corresponding to BCMA-17 as set forth in Table 11A.
846. The MBM of embodiment 745, wherein if TAA 1 is BCMA, ABM1 comprises a scFv comprising the amino acid sequence of scFv corresponding to BCMA-18 as set forth in Table 11A.
847. The MBM of embodiment 745, wherein if TAA 1 is BCMA, ABM1 comprises a scFv comprising the amino acid sequence of scFv corresponding to BCMA-19 as set forth in Table 11A.
848. The MBM of embodiment 745, wherein if TAA 1 is BCMA, ABM1 comprises a scFv comprising the amino acid sequence of scFv corresponding to BCMA-20 as set forth in Table 11A.
849. The MBM of embodiment 745, wherein if TAA 1 is BCMA, ABM1 comprises a scFv comprising the amino acid sequence of scFv corresponding to BCMA-21 as set forth in Table 11A.
850. The MBM of embodiment 745, wherein if TAA 1 is BCMA, ABM1 comprises a scFv comprising the amino acid sequence of scFv corresponding to BCMA-22 as set forth in Table 11A.
851. The MBM of embodiment 745, wherein if TAA 1 is BCMA, ABM1 comprises a scFv comprising the amino acid sequence of scFv corresponding to BCMA-23 as set forth in Table 11A.
852. The MBM of embodiment 745, wherein if TAA 1 is BCMA, ABM1 comprises a scFv comprising the amino acid sequence of scFv corresponding to BCMA-24 as set forth in Table 11A.
853. The MBM of embodiment 745, wherein if TAA 1 is BCMA, ABM1 comprises a scFv comprising the amino acid sequence of scFv corresponding to BCMA-25 as set forth in Table 11A.
854. The MBM of embodiment 745, wherein if TAA 1 is BCMA, ABM1 comprises a scFv comprising the amino acid sequence of scFv corresponding to BCMA-26 as set forth in Table 11A.
855. The MBM of embodiment 745, wherein if TAA 1 is BCMA, ABM1 comprises a scFv comprising the amino acid sequence of scFv corresponding to BCMA-27 as set forth in Table 11A.
856. The MBM of embodiment 745, wherein if TAA 1 is BCMA, ABM1 comprises a scFv comprising the amino acid sequence of scFv corresponding to BCMA-28 as set forth in Table 11A.
857. The MBM of embodiment 745, wherein if TAA 1 is BCMA, ABM1 comprises a scFv comprising the amino acid sequence of scFv corresponding to BCMA-29 as set forth in Table 11A.
858. The MBM of embodiment 745, wherein if TAA 1 is BCMA, ABM1 comprises a scFv comprising the amino acid sequence of scFv corresponding to BCMA-30 as set forth in Table 11A.
859. The MBM of embodiment 745, wherein if TAA 1 is BCMA, ABM1 comprises a scFv comprising the amino acid sequence of scFv corresponding to BCMA-31 as set forth in Table 11A.
860. The MBM of embodiment 745, wherein if TAA 1 is BCMA, ABM1 comprises a scFv comprising the amino acid sequence of scFv corresponding to BCMA-32 as set forth in Table 11A.
861. The MBM of embodiment 745, wherein if TAA 1 is BCMA, ABM1 comprises a scFv comprising the amino acid sequence of scFv corresponding to BCMA-33 as set forth in Table 11A.
862. The MBM of embodiment 745, wherein if TAA 1 is BCMA, ABM1 comprises a scFv comprising the amino acid sequence of scFv corresponding to BCMA-34 as set forth in Table 11A.
863. The MBM of embodiment 745, wherein if TAA 1 is BCMA, ABM1 comprises a scFv comprising the amino acid sequence of scFv corresponding to BCMA-35 as set forth in Table 11A.
864. The MBM of embodiment 745, wherein if TAA 1 is BCMA, ABM1 comprises a scFv comprising the amino acid sequence of scFv corresponding to BCMA-36 as set forth in Table 11A.
865. The MBM of embodiment 745, wherein if TAA 1 is BCMA, ABM1 comprises a scFv comprising the amino acid sequence of scFv corresponding to BCMA-37 as set forth in Table 11A.
866. The MBM of embodiment 745, wherein if TAA 1 is BCMA, ABM1 comprises a scFv comprising the amino acid sequence of scFv corresponding to BCMA-38 as set forth in Table 11A.
867. The MBM of embodiment 745, wherein if TAA 1 is BCMA, ABM1 comprises a scFv comprising the amino acid sequence of scFv corresponding to BCMA-39 as set forth in Table 11A.
868. The MBM of embodiment 745, wherein if TAA 1 is BCMA, ABM1 comprises a scFv comprising the amino acid sequence of scFv corresponding to BCMA-40 as set forth in Table 11A.
869. The MBM of embodiment 745, wherein if TAA 1 is CD19, ABM1 comprises:
   (a) a CDR-H1 having the amino acid sequence of the CDR designated as CD19-H1;
   (b) a CDR-H2 having the amino acid sequence of any one of the CDRs designated as CD19-H2A, HD19-H2B, CD19-H2C and CD19-H2D;
   (c) a CDR-H3 having the amino acid sequence of the CDR designated as CD19-H3;
   (d) a CDR-L1 having the amino acid sequence of the CDR designated as CD19-L1;
   (e) a CDR-L2 having the amino acid sequence of the CDR designated as CD19-L2; and
   (f) a CDR-L3 having the amino acid sequence of the CDR designated as CD19-L23.
870. The MBM of embodiment 869, wherein ABM1 comprises:
   (a) a VH having the amino acid sequence of any one of the VH's designated as CD19-VHA, CD19-VHB, CD19-VHC, and CD19-VHD; and
   (b) a VL having the amino acid sequence of any one of the VL's designated as CD19-VLA and CD19-VLB.
871. The MBM of embodiment 745, wherein if TAA 1 is CD19, ABM1 comprises heavy chain CDRs having the amino acid sequences of CD19-H1, CD19-H2A, and CD19-H3 as set forth in Table 12 and light chain CDRs having the amino acid sequences of CD19-1, CD19-L2, and CD19-L3 as set forth in Table 12.
872. The MBM of embodiment 745, wherein if TAA 1 is CD19, ABM1 comprises a heavy chain variable region having the amino acid sequences of VHA as set forth in Table 12 and a light chain variable region having the amino acid sequences of VLA as set forth in Table 12.
873. The MBM of embodiment 745, wherein if TAA 1 is CD19, ABM1 comprises heavy chain CDRs having the amino acid sequences of CD19-H1, CD19-H2B, and CD19-H3 as set forth in Table 12 and light chain CDRs having the amino acid sequences of CD19-1, CD19-L2, and CD19-L3 as set forth in Table 12.
874. The MBM of embodiment 745, wherein if TAA 1 is CD19, ABM1 comprises a heavy chain variable region having the amino acid sequences of VHB as set forth in Table 12 and a light chain variable region having the amino acid sequences of VLB as set forth in Table 12.
875. The MBM of embodiment 745, wherein if TAA 1 is CD19, ABM1 comprises heavy chain CDRs having the amino acid sequences of CD19-H1, CD19-H2C, and CD19-H3 as set forth in Table 12 and light chain CDRs having the amino acid sequences of CD19-1, CD19-L2, and CD19-L3 as set forth in Table 12.
876. The MBM of embodiment 745, wherein if TAA 1 is CD19, ABM1 comprises a heavy chain variable region having the amino acid sequences of VHC as set forth in Table 12 and a light chain variable region having the amino acid sequences of VLB as set forth in Table 2.
877. The MBM of embodiment 745, wherein if TAA 1 is CD19, ABM1 comprises heavy chain CDRs having the amino acid sequences of CD19-H1, CD19-H2D, and CD19-H3 as set forth in Table 12 and light chain CDRs having the amino acid sequences of CD19-1, CD19-L2, and CD19-L3 as set forth in Table 12.
878. The MBM of embodiment 745, wherein if TAA 1 is CD19, ABM1comprises a heavy chain variable region having the amino acid sequences of VHD as set forth in Table 12 and a light chain variable region having the amino acid sequences of VLB as set forth in Table 12.
879. The MBM of embodiment 745, wherein if TAA 1 is CD19, ABM1 comprises a scFv comprising the amino acid sequence of CD19-scFv1 as set forth in Table 12.
880. The MBM of embodiment 745, wherein if TAA 1 is CD19, ABM1 comprises a scFv comprising the amino acid sequence of CD19-scFv2 as set forth in Table 12.
881. The MBM of embodiment 745, wherein if TAA 1 is CD19, ABM1 comprises a scFv comprising the amino acid sequence of CD19-scFv3 as set forth in Table 12.
882. The MBM of embodiment 745, wherein if TAA 1 is CD19, ABM1 comprises a scFv comprising the amino acid sequence of CD19-scFv4 as set forth in Table 12.
883. The MBM of embodiment 745, wherein if TAA 1 is CD19, ABM1 comprises a scFv comprising the amino acid sequence of CD19-scFv5 as set forth in Table 12.
884. The MBM of embodiment 745, wherein if TAA 1 is CD19, ABM1 comprises a scFv comprising the amino acid sequence of CD19-scFv6 as set forth in Table 12.
885. The MBM of embodiment 745, wherein if TAA 1 is CD19, ABM1 comprises a scFv comprising the amino acid sequence of CD19-scFv7 as set forth in Table 12.
886. The MBM of embodiment 745, wherein if TAA 1 is CD19, ABM1 comprises a scFv comprising the amino acid sequence of CD19-scFv8 as set forth in Table 12.
887. The MBM of embodiment 745, wherein if TAA 1 is CD19, ABM1 comprises a scFv comprising the amino acid sequence of CD19-scFv9 as set forth in Table 12.
888. The MBM of embodiment 745, wherein if TAA 1 is CD19, ABM1 comprises a scFv comprising the amino acid sequence of CD19-scFv10 as set forth in Table 12.
889. The MBM of embodiment 745, wherein if TAA 1 is CD19, ABM1 comprises a scFv comprising the amino acid sequence of CD19-scFv11 as set forth in Table 12.
890. The MBM of embodiment 745, wherein if TAA 1 is CD19, ABM1 comprises a scFv comprising the amino acid sequence of CD19-scFv12 as set forth in Table 12.
891. The MBM of any one of embodiments 1 to 737, wherein ABM1 is a non-immunoglobulin scaffold-based ABM.
892. The MBM of embodiment 891, wherein ABM1 is a Kunitz domain, an Adnexin, an Affibody, a DARPin, an Avimer, an Anticalin, a Lipocalin, a Centyrin, a Versabody, a Knottin, an Adnectin, a Pronectin, an Affitin/Nanofitin, an Affilin, an Atrimer/Tetranectin, a bicyclic peptide, a cys-knot, a Fn3 scaffold, an Obody, a Tn3, an Affimer, BD, an Adhiron, a Duocalin, an Alphabody, an Armadillo Repeat Protein, a Repebody, or a Fynomer.
893. The MBM of any one of embodiments 1 to 892, wherein ABM2 is an immunoglobulin scaffold-based ABM.
894. The MBM of embodiment 893, wherein ABM2 is an anti-TAA 2 antibody, an antibody fragment, an scFv, a dsFv, a Fv, a Fab, an scFab, a (Fab′)₂, a single domain antibody (SDAB), a VH or VL domain, or a camelid VHH domain.
895. The MBM of embodiment 894, wherein ABM2 is an scFv.
896. The MBM of embodiment 894, wherein ABM2 is a Fab.
897. The MBM of embodiment 896, wherein the Fab is a Fab heterodimer.
898. The MBM of any one of embodiments 893 to 897, wherein ABM2 comprises a binding sequence described in Table 10.
899. The MBM of embodiment 898, wherein ABM2 comprises the CDRs or variable region sequences of the antibodies set forth in Table 10.
900. The MBM of any one of embodiments 893 to 897, wherein:
   (a) if TAA 2 is BCMA, ABM2 optionally comprises a binding sequence described in Table 11; and
   (b) if TAA 2 is CD19, ABM2 optionally comprises a binding sequence described in Table 12.
901. The MBM of embodiment 900, wherein if TAA 2 is BCMA, ABM2 comprises the CDR sequences of BCMA-1.
902. The MBM of embodiment 900, wherein if TAA 2 is BCMA, ABM2 comprises the CDR sequences of BCMA-2.
903. The MBM of embodiment 900, wherein if TAA 2 is BCMA, ABM2 comprises the CDR sequences of BCMA-3.
904. The MBM of embodiment 900, wherein if TAA 2 is BCMA, ABM2 comprises the CDR sequences of BCMA-4.
905. The MBM of embodiment 900, wherein if TAA 2 is BCMA, ABM2 comprises the CDR sequences of BCMA-5.
906. The MBM of embodiment 900, wherein if TAA 2 is BCMA, ABM2 comprises the CDR sequences of BCMA-6.
907. The MBM of embodiment 900, wherein if TAA 2 is BCMA, ABM2 comprises the CDR sequences of BCMA-7.
908. The MBM of embodiment 900, wherein if TAA 2 is BCMA, ABM2 comprises the CDR sequences of BCMA-8.
909. The MBM of embodiment 900, wherein if TAA 2 is BCMA, ABM2 comprises the CDR sequences of BCMA-9.
910. The MBM of embodiment 900, wherein if TAA 2 is BCMA, ABM2 comprises the CDR sequences of BCMA-10.
911. The MBM of embodiment 900, wherein if TAA 2 is BCMA, ABM2 comprises the CDR sequences of BCMA-11.
912. The MBM of embodiment 900, wherein if TAA 2 is BCMA, ABM2 comprises the CDR sequences of BCMA-12.
913. The MBM of embodiment 900, wherein if TAA 2 is BCMA, ABM2 comprises the CDR sequences of BCMA-13.
914. The MBM of embodiment 900, wherein if TAA 2 is BCMA, ABM2 comprises the CDR sequences of BCMA-14.
915. The MBM of embodiment 900, wherein if TAA 2 is BCMA, ABM2 comprises the CDR sequences of BCMA-15.
916. The MBM of embodiment 900, wherein if TAA 2 is BCMA, ABM2 comprises the CDR sequences of BCMA-16.
917. The MBM of embodiment 900, wherein if TAA 2 is BCMA, ABM2 comprises the CDR sequences of BCMA-17.
918. The MBM of embodiment 900, wherein if TAA 2 is BCMA, ABM2 comprises the CDR sequences of BCMA-18.
919. The MBM of embodiment 900, wherein if TAA 2 is BCMA, ABM2 comprises the CDR sequences of BCMA-19.
920. The MBM of embodiment 900, wherein if TAA 2 is BCMA, ABM2 comprises the CDR sequences of BCMA-20.
921. The MBM of embodiment 900, wherein if TAA 2 is BCMA, ABM2 comprises the CDR sequences of BCMA-21.
922. The MBM of embodiment 900, wherein if TAA 2 is BCMA, ABM2 comprises the CDR sequences of BCMA-22.
923. The MBM of embodiment 900, wherein if TAA 2 is BCMA, ABM2 comprises the CDR sequences of BCMA-23.
924. The MBM of embodiment 900, wherein if TAA 2 is BCMA, ABM2 comprises the CDR sequences of BCMA-24.
925. The MBM of embodiment 900, wherein if TAA 2 is BCMA, ABM2 comprises the CDR sequences of BCMA-25.
926. The MBM of embodiment 900, wherein if TAA 2 is BCMA, ABM2 comprises the CDR sequences of BCMA-26.
927. The MBM of embodiment 900, wherein if TAA 2 is BCMA, ABM2 comprises the CDR sequences of BCMA-27.
928. The MBM of embodiment 900, wherein if TAA 2 is BCMA, ABM2 comprises the CDR sequences of BCMA-28.
929. The MBM of embodiment 900, wherein if TAA 2 is BCMA, ABM2 comprises the CDR sequences of BCMA-29.
930. The MBM of embodiment 900, wherein if TAA 2 is BCMA, ABM2 comprises the CDR sequences of BCMA-30.
931. The MBM of embodiment 900, wherein if TAA 2 is BCMA, ABM2 comprises the CDR sequences of BCMA-31.
932. The MBM of embodiment 900, wherein if TAA 2 is BCMA, ABM2 comprises the CDR sequences of BCMA-32.
933. The MBM of embodiment 900, wherein if TAA 2 is BCMA, ABM2 comprises the CDR sequences of BCMA-33.
934. The MBM of embodiment 900, wherein if TAA 2 is BCMA, ABM2 comprises the CDR sequences of BCMA-34.
935. The MBM of embodiment 900, wherein if TAA 2 is BCMA, ABM2 comprises the CDR sequences of BCMA-35.
936. The MBM of embodiment 900, wherein if TAA 2 is BCMA, ABM2 comprises the CDR sequences of BCMA-36.
937. The MBM of embodiment 900, wherein if TAA 2 is BCMA, ABM2 comprises the CDR sequences of BCMA-37.
938. The MBM of embodiment 900, wherein if TAA 2 is BCMA, ABM2 comprises the CDR sequences of BCMA-38.
939. The MBM of embodiment 900, wherein if TAA 2 is BCMA, ABM2 comprises the CDR sequences of BCMA-39.
940. The MBM of embodiment 900, wherein if TAA 2 is BCMA, ABM2 comprises the CDR sequences of BCMA-40.
941. The MBM of any one of embodiments 901 to 940, wherein the CDRs are defined by Kabat numbering, as set forth in Table 11B and 11E.
942. The MBM of any one of embodiments 901 to 940, wherein the CDRs are defined by Chothia numbering, as set forth in Table 11C and 11F.
943. MBM of any one of embodiments 901 to 940, wherein the CDRs are defined by a combination of Kabat and Chothia numbering, as set forth in Table 11D and 11G.
944. The MBM of embodiment 900, wherein if TAA 2 is BCMA, ABM2 comprises the heavy and light chain variable sequences of BCMA-1, as set forth in Table 11A.
945. The MBM of embodiment 900, wherein if TAA 2 is BCMA, ABM2 comprises the heavy and light chain variable sequences of BCMA-2, as set forth in Table 11A.
946. The MBM of embodiment 900, wherein if TAA 2 is BCMA, ABM2 comprises the heavy and light chain variable sequences of BCMA-3, as set forth in Table 11A.
947. The MBM of embodiment 900, wherein if TAA 2 is BCMA, ABM2 comprises the heavy and light chain variable sequences of BCMA-4, as set forth in Table 11A.
948. The MBM of embodiment 900, wherein if TAA 2 is BCMA, ABM2 comprises the heavy and light chain variable sequences of BCMA-5, as set forth in Table 11A.
949. The MBM of embodiment 900, wherein if TAA 2 is BCMA, ABM2 comprises the heavy and light chain variable sequences of BCMA-6, as set forth in Table 11A.
950. The MBM of embodiment 900, wherein if TAA 2 is BCMA, ABM2 comprises the heavy and light chain variable sequences of BCMA-7, as set forth in Table 11A.
951. The MBM of embodiment 900, wherein if TAA 2 is BCMA, ABM2 comprises the heavy and light chain variable sequences of BCMA-8, as set forth in Table 11A.
952. The MBM of embodiment 900, wherein if TAA 2 is BCMA, ABM2 comprises the heavy and light chain variable sequences of BCMA-9, as set forth in Table 11A.
953. The MBM of embodiment 900, wherein if TAA 2 is BCMA, ABM2 comprises the heavy and light chain variable sequences of BCMA-10, as set forth in Table 11A.
954. The MBM of embodiment 900, wherein if TAA 2 is BCMA, ABM2 comprises the heavy and light chain variable sequences of BCMA-11, as set forth in Table 11A.
955. The MBM of embodiment 900, wherein if TAA 2 is BCMA, ABM2 comprises the heavy and light chain variable sequences of BCMA-12, as set forth in Table 11A.
956. The MBM of embodiment 900, wherein if TAA 2 is BCMA, ABM2 comprises the heavy and light chain variable sequences of BCMA-13, as set forth in Table 11A.
957. The MBM of embodiment 900, wherein if TAA 2 is BCMA, ABM2 comprises the heavy and light chain variable sequences of BCMA-14, as set forth in Table 11A.
958. The MBM of embodiment 900, wherein if TAA 2 is BCMA, ABM2 comprises the heavy and light chain variable sequences of BCMA-15, as set forth in Table 11A.
959. The MBM of embodiment 900, wherein if TAA 2 is BCMA, ABM2 comprises the heavy and light chain variable sequences of BCMA-16, as set forth in Table 11A.
960. The MBM of embodiment 900, wherein if TAA 2 is BCMA, ABM2 comprises the heavy and light chain variable sequences of BCMA-17, as set forth in Table 11A.
961. The MBM of embodiment 900, wherein if TAA 2 is BCMA, ABM2 comprises the heavy and light chain variable sequences of BCMA-18, as set forth in Table 11A.
962. The MBM of embodiment 900, wherein if TAA 2 is BCMA, ABM2 comprises the heavy and light chain variable sequences of BCMA-19, as set forth in Table 11A.
963. The MBM of embodiment 900, wherein if TAA 2 is BCMA, ABM2 comprises the heavy and light chain variable sequences of BCMA-20, as set forth in Table 11A.
964. The MBM of embodiment 900, wherein if TAA 2 is BCMA, ABM2 comprises the heavy and light chain variable sequences of BCMA-21, as set forth in Table 11A.
965. The MBM of embodiment 900, wherein if TAA 2 is BCMA, ABM2 comprises the heavy and light chain variable sequences of BCMA-22, as set forth in Table 11A.
966. The MBM of embodiment 900, wherein if TAA 2 is BCMA, ABM2 comprises the heavy and light chain variable sequences of BCMA-23, as set forth in Table 11A.
967. The MBM of embodiment 900, wherein if TAA 2 is BCMA, ABM2 comprises the heavy and light chain variable sequences of BCMA-24, as set forth in Table 11A.
968. The MBM of embodiment 900, wherein if TAA 2 is BCMA, ABM2 comprises the heavy and light chain variable sequences of BCMA-25, as set forth in Table 11A.
969. The MBM of embodiment 900, wherein if TAA 2 is BCMA, ABM2 comprises the heavy and light chain variable sequences of BCMA-26, as set forth in Table 11A.
970. The MBM of embodiment 900, wherein if TAA 2 is BCMA, ABM2 comprises the heavy and light chain variable sequences of BCMA-27, as set forth in Table 11A.
971. The MBM of embodiment 900, wherein if TAA 2 is BCMA, ABM2 comprises the heavy and light chain variable sequences of BCMA-28, as set forth in Table 11A.
972. The MBM of embodiment 900, wherein if TAA 2 is BCMA, ABM2 comprises the heavy and light chain variable sequences of BCMA-29, as set forth in Table 11A.
973. The MBM of embodiment 900, wherein if TAA 2 is BCMA, ABM2 comprises the heavy and light chain variable sequences of BCMA-30, as set forth in Table 11A.
974. The MBM of embodiment 900, wherein if TAA 2 is BCMA, ABM2 comprises the heavy and light chain variable sequences of BCMA-31, as set forth in Table 11A.
975. The MBM of embodiment 900, wherein if TAA 2 is BCMA, ABM2 comprises the heavy and light chain variable sequences of BCMA-32, as set forth in Table 11A.
976. The MBM of embodiment 900, wherein if TAA 2 is BCMA, ABM2 comprises the heavy and light chain variable sequences of BCMA-33, as set forth in Table 11A.
977. The MBM of embodiment 900, wherein if TAA 2 is BCMA, ABM2 comprises the heavy and light chain variable sequences of BCMA-34, as set forth in Table 11A.
978. The MBM of embodiment 900, wherein if TAA 2 is BCMA, ABM2 comprises the heavy and light chain variable sequences of BCMA-35, as set forth in Table 11A.
979. The MBM of embodiment 900, wherein if TAA 2 is BCMA, ABM2 comprises the heavy and light chain variable sequences of BCMA-36, as set forth in Table 11A.
980. The MBM of embodiment 900, wherein if TAA 2 is BCMA, ABM2 comprises the heavy and light chain variable sequences of BCMA-37, as set forth in Table 11A.
981. The MBM of embodiment 900, wherein if TAA 2 is BCMA, ABM2 comprises the heavy and light chain variable sequences of BCMA-38, as set forth in Table 11A.
982. The MBM of embodiment 900, wherein if TAA 2 is BCMA, ABM2 comprises the heavy and light chain variable sequences of BCMA-39, as set forth in Table 11A.
983. The MBM of embodiment 900, wherein if TAA 2 is BCMA, ABM2 comprises the heavy and light chain variable sequences of BCMA-40, as set forth in Table 11A.
984. The MBM of embodiment 900, wherein if TAA 2 is BCMA, ABM2 comprises a scFv comprising the amino acid sequence of scFv corresponding to BCMA-1 as set forth in Table 11A.
985. The MBM of embodiment 900, wherein if TAA 2 is BCMA, ABM2 comprises a scFv comprising the amino acid sequence of scFv corresponding to BCMA-2 as set forth in Table 11A.
986. The MBM of embodiment 900, wherein if TAA 2 is BCMA, ABM2 comprises a scFv comprising the amino acid sequence of scFv corresponding to BCMA-3 as set forth in Table 11A.
987. The MBM of embodiment 900, wherein if TAA 2 is BCMA, ABM2 comprises a scFv comprising the amino acid sequence of scFv corresponding to BCMA-4 as set forth in Table 11A.
988. The MBM of embodiment 900, wherein if TAA 2 is BCMA, ABM2 comprises a scFv comprising the amino acid sequence of scFv corresponding to BCMA-5 as set forth in Table 11A.
989. The MBM of embodiment 900, wherein if TAA 2 is BCMA, ABM2 comprises a scFv comprising the amino acid sequence of scFv corresponding to BCMA-6 as set forth in Table 11A.
990. The MBM of embodiment 900, wherein if TAA 2 is BCMA, ABM2 comprises a scFv comprising the amino acid sequence of scFv corresponding to BCMA-7 as set forth in Table 11A.
991. The MBM of embodiment 900, wherein if TAA 2 is BCMA, ABM2 comprises a scFv comprising the amino acid sequence of scFv corresponding to BCMA-8 as set forth in Table 11A.
992. The MBM of embodiment 900, wherein if TAA 2 is BCMA, ABM2 comprises a scFv comprising the amino acid sequence of scFv corresponding to BCMA-9 as set forth in Table 11A.
993. The MBM of embodiment 900, wherein if TAA 2 is BCMA, ABM2 comprises a scFv comprising the amino acid sequence of scFv corresponding to BCMA-10 as set forth in Table 11A.
994. The MBM of embodiment 900, wherein if TAA 2 is BCMA, ABM2 comprises a scFv comprising the amino acid sequence of scFv corresponding to BCMA-11 as set forth in Table 11A.
995. The MBM of embodiment 900, wherein if TAA 2 is BCMA, ABM2 comprises a scFv comprising the amino acid sequence of scFv corresponding to BCMA-12 as set forth in Table 11A.
996. The MBM of embodiment 900, wherein if TAA 2 is BCMA, ABM2 comprises a scFv comprising the amino acid sequence of scFv corresponding to BCMA-13 as set forth in Table 11A.
997. The MBM of embodiment 900, wherein if TAA 2 is BCMA, ABM2 comprises a scFv comprising the amino acid sequence of scFv corresponding to BCMA-14 as set forth in Table 11A.
998. The MBM of embodiment 900, wherein if TAA 2 is BCMA, ABM2 comprises a scFv comprising the amino acid sequence of scFv corresponding to BCMA-15 as set forth in Table 11A.
999. The MBM of embodiment 900, wherein if TAA 2 is BCMA, ABM2 comprises a scFv comprising the amino acid sequence of scFv corresponding to BCMA-16 as set forth in Table 11A.
1000. The MBM of embodiment 900, wherein if TAA 2 is BCMA, ABM2 comprises a scFv comprising the amino acid sequence of scFv corresponding to BCMA-17 as set forth in Table 11A.
1001. The MBM of embodiment 900, wherein if TAA 2 is BCMA, ABM2 comprises a scFv comprising the amino acid sequence of scFv corresponding to BCMA-18 as set forth in Table 11A.
1002. The MBM of embodiment 900, wherein if TAA 2 is BCMA, ABM2 comprises a scFv comprising the amino acid sequence of scFv corresponding to BCMA-19 as set forth in Table 11A.
1003. The MBM of embodiment 900, wherein if TAA 2 is BCMA, ABM2 comprises a scFv comprising the amino acid sequence of scFv corresponding to BCMA-20 as set forth in Table 11A.
1004. The MBM of embodiment 900, wherein if TAA 2 is BCMA, ABM2 comprises a scFv comprising the amino acid sequence of scFv corresponding to BCMA-21 as set forth in Table 11A.
1005. The MBM of embodiment 900, wherein if TAA 2 is BCMA, ABM2 comprises a scFv comprising the amino acid sequence of scFv corresponding to BCMA-22 as set forth in Table 11A.
1006. The MBM of embodiment 900, wherein if TAA 2 is BCMA, ABM2 comprises a scFv comprising the amino acid sequence of scFv corresponding to BCMA-23 as set forth in Table 11A.
1007. The MBM of embodiment 900, wherein if TAA 2 is BCMA, ABM2 comprises a scFv comprising the amino acid sequence of scFv corresponding to BCMA-24 as set forth in Table 11A.
1008. The MBM of embodiment 900, wherein if TAA 2 is BCMA, ABM2 comprises a scFv comprising the amino acid sequence of scFv corresponding to BCMA-25 as set forth in Table 11A.
1009. The MBM of embodiment 900, wherein if TAA 2 is BCMA, ABM2 comprises a scFv comprising the amino acid sequence of scFv corresponding to BCMA-26 as set forth in Table 11A.
1010. The MBM of embodiment 900, wherein if TAA 2 is BCMA, ABM2 comprises a scFv comprising the amino acid sequence of scFv corresponding to BCMA-27 as set forth in Table 11A.
1011. The MBM of embodiment 900, wherein if TAA 2 is BCMA, ABM2 comprises a scFv comprising the amino acid sequence of scFv corresponding to BCMA-28 as set forth in Table 11A.
1012. The MBM of embodiment 900, wherein if TAA 2 is BCMA, ABM2 comprises a scFv comprising the amino acid sequence of scFv corresponding to BCMA-29 as set forth in Table 11A.
1013. The MBM of embodiment 900, wherein if TAA 2 is BCMA, ABM2 comprises a scFv comprising the amino acid sequence of scFv corresponding to BCMA-30 as set forth in Table 11A.
1014. The MBM of embodiment 900, wherein if TAA 2 is BCMA, ABM2 comprises a scFv comprising the amino acid sequence of scFv corresponding to BCMA-31 as set forth in Table 11A.
1015. The MBM of embodiment 900, wherein if TAA 2 is BCMA, ABM2 comprises a scFv comprising the amino acid sequence of scFv corresponding to BCMA-32 as set forth in Table 11A.
1016. The MBM of embodiment 900, wherein if TAA 2 is BCMA, ABM2 comprises a scFv comprising the amino acid sequence of scFv corresponding to BCMA-33 as set forth in Table 11A.
1017. The MBM of embodiment 900, wherein if TAA 2 is BCMA, ABM2 comprises a scFv comprising the amino acid sequence of scFv corresponding to BCMA-34 as set forth in Table 11A.
1018. The MBM of embodiment 900, wherein if TAA 2 is BCMA, ABM2 comprises a scFv comprising the amino acid sequence of scFv corresponding to BCMA-35 as set forth in Table 11A.
1019. The MBM of embodiment 900, wherein if TAA 2 is BCMA, ABM2 comprises a scFv comprising the amino acid sequence of scFv corresponding to BCMA-36 as set forth in Table 11A.
1020. The MBM of embodiment 900, wherein if TAA 2 is BCMA, ABM2 comprises a scFv comprising the amino acid sequence of scFv corresponding to BCMA-37 as set forth in Table 11A.
1021. The MBM of embodiment 900, wherein if TAA 2 is BCMA, ABM2 comprises a scFv comprising the amino acid sequence of scFv corresponding to BCMA-38 as set forth in Table 11A.
1022. The MBM of embodiment 900, wherein if TAA 2 is BCMA, ABM2 comprises a scFv comprising the amino acid sequence of scFv corresponding to BCMA-39 as set forth in Table 11A.
1023. The MBM of embodiment 900, wherein if TAA 2 is BCMA, ABM2 comprises a scFv comprising the amino acid sequence of scFv corresponding to BCMA-40 as set forth in Table 11A.
1024. The MBM of embodiment 900, wherein if TAA 2 is CD19, ABM2 comprises:
   (a) a CDR-H1 having the amino acid sequence of the CDR designated as CD19-H1;
   (b) a CDR-H2 having the amino acid sequence of any one of the CDRs designated as CD19-H2A, HD19-H2B, CD19-H2C and CD19-H2D;
   (c) a CDR-H3 having the amino acid sequence of the CDR designated as CD19-H3;
   (d) a CDR-L1 having the amino acid sequence of the CDR designated as CD19-L1;
   (e) a CDR-L2 having the amino acid sequence of the CDR designated as CD19-L2; and
   (f) a CDR-L3 having the amino acid sequence of the CDR designated as CD19-L23.
1025. The MBM of embodiment 1024, wherein ABM2 comprises:
   (a) a VH having the amino acid sequence of any one of the VH's designated as CD19-VHA, CD19-VHB, CD19-VHC, and CD19-VHD; and
   (b) a VL having the amino acid sequence of any one of the VL's designated as CD19-VLA and CD19-VLB.
1026. The MBM of embodiment 900, wherein if TAA 2 is CD19, ABM1 comprises heavy chain CDRs having the amino acid sequences of CD19-H1, CD19-H2A, and CD19-H3 as set forth in Table 12 and light chain CDRs having the amino acid sequences of CD19-1, CD19-L2, and CD19-L3 as set forth in Table 12.
1027. The MBM of embodiment 900, wherein if TAA 2 is CD19, ABM1 comprises a heavy chain variable region having the amino acid sequences of VHA as set forth in Table 12 and a light chain variable region having the amino acid sequences of VLA as set forth in Table 12.
1028. The MBM of embodiment 900, wherein if TAA 2 is CD19, ABM1 comprises heavy chain CDRs having the amino acid sequences of CD19-H1, CD19-H2B, and CD19-H3 as set forth in Table 12 and light chain CDRs having the amino acid sequences of CD19-1, CD19-L2, and CD19-L3 as set forth in Table 12.
1029. The MBM of embodiment 900, wherein if TAA 2 is CD19, ABM1 comprises a heavy chain variable region having the amino acid sequences of VHB as set forth in Table 12 and a light chain variable region having the amino acid sequences of VLB as set forth in Table 12.
1030. The MBM of embodiment 900, wherein if TAA 2 is CD19, ABM1 comprises heavy chain CDRs having the amino acid sequences of CD19-H1, CD19-H2C, and CD19-H3 as set forth in Table 12 and light chain CDRs having the amino acid sequences of CD19-1, CD19-L2, and CD19-L3 as set forth in Table 12.
1031. The MBM of embodiment 900, wherein if TAA 2 is CD19, ABM1comprises a heavy chain variable region having the amino acid sequences of VHC as set forth in Table 12 and a light chain variable region having the amino acid sequences of VLB as set forth in Table 2.
1032. The MBM of embodiment 900, wherein if TAA 2 is CD19, ABM1 comprises heavy chain CDRs having the amino acid sequences of CD19-H1, CD19-H2D, and CD19-H3 as set forth in Table 12 and light chain CDRs having the amino acid sequences of CD19-1, CD19-L2, and CD19-L3 as set forth in Table 12.
1033. The MBM of embodiment 900, wherein if TAA 2 is CD19, ABM1 comprises a heavy chain variable region having the amino acid sequences of VHD as set forth in Table 12 and a light chain variable region having the amino acid sequences of VLB as set forth in Table 12.
1034. The MBM of embodiment 900, wherein if TAA 2 is CD19, ABM2 comprises a scFv comprising the amino acid sequence of CD19-scFv1 as set forth in Table 12.
1035. The MBM of embodiment 900, wherein if TAA 2 is CD19, ABM2 comprises a scFv comprising the amino acid sequence of CD19-scFv2 as set forth in Table 12.
1036. The MBM of embodiment 900, wherein if TAA 2 is CD19, ABM2 comprises a scFv comprising the amino acid sequence of CD19-scFv3 as set forth in Table 12.
1037. The MBM of embodiment 900, wherein if TAA 2 is CD19, ABM2 comprises a scFv comprising the amino acid sequence of CD19-scFv4 as set forth in Table 12.
1038. The MBM of embodiment 900, wherein if TAA 2 is CD19, ABM2 comprises a scFv comprising the amino acid sequence of CD19-scFv5 as set forth in Table 12.
1039. The MBM of embodiment 900, wherein if TAA 2 is CD19, ABM2 comprises a scFv comprising the amino acid sequence of CD19-scFv6 as set forth in Table 12.
1040. The MBM of embodiment 900, wherein if TAA 2 is CD19, ABM2 comprises a scFv comprising the amino acid sequence of CD19-scFv7 as set forth in Table 12.
1041. The MBM of embodiment 900, wherein if TAA 2 is CD19, ABM2 comprises a scFv comprising the amino acid sequence of CD19-scFv8 as set forth in Table 12.
1042. The MBM of embodiment 900, wherein if TAA 2 is CD19, ABM2 comprises a scFv comprising the amino acid sequence of CD19-scFv9 as set forth in Table 12.
1043. The MBM of embodiment 900, wherein if TAA 2 is CD19, ABM2 comprises a scFv comprising the amino acid sequence of CD19-scFv10 as set forth in Table 12.
1044. The MBM of embodiment 900, wherein if TAA 2 is CD19, ABM2 comprises a scFv comprising the amino acid sequence of CD19-scFv11 as set forth in Table 12.
1045. The MBM of embodiment 900, wherein if TAA 2 is CD19, ABM2 comprises a scFv comprising the amino acid sequence of CD19-scFv12 as set forth in Table 12.
1046. The MBM of any one of embodiments 1 to 892, wherein ABM2 is a non-immunoglobulin scaffold-based ABM.
1047. The MBM of embodiment 1046, wherein ABM2 is a Kunitz domain, an Adnexin, an Affibody, a DARPin, an Avimer, an Anticalin, a Lipocalin, a Centyrin, a Versabody, a Knottin, an Adnectin, a Pronectin, an Affitin/Nanofitin, an Affilin, an Atrimer/Tetranectin, a bicyclic peptide, a cys-knot, a Fn3 scaffold, an Obody, a Tn3, an Affimer, BD, an Adhiron, a Duocalin, an Alphabody, an Armadillo Repeat Protein, a Repebody, or a Fynomer.
1048. The MBM of any one of embodiments 1 to 1047, wherein the component of the TCR complex is CD3.
1049. The MBM of embodiment 1048, wherein ABM3 is an anti-CD3 antibody or an antigen-binding domain thereof.
1050. The MBM of embodiment 1048 or embodiment 1049, wherein ABM3 comprises the CDR sequences of CD3-1.
1051. The MBM of embodiment 1048 or embodiment 1049, wherein ABM3 comprises the CDR sequences of CD3-2.
1052. The MBM of embodiment 1048 or embodiment 1049, wherein ABM3 comprises the CDR sequences of CD3-3.
1053. The MBM of embodiment 1048 or embodiment 1049, wherein ABM3 comprises the CDR sequences of CD3-4.
1054. The MBM of embodiment 1048 or embodiment 1049, wherein ABM3 comprises the CDR sequences of CD3-5.
1055. The MBM of embodiment 1048 or embodiment 1049, wherein ABM3 comprises the CDR sequences of CD3-6.
1056. The MBM of embodiment 1048 or embodiment 1049, wherein ABM3 comprises the CDR sequences of CD3-7.
1057. The MBM of embodiment 1048 or embodiment 1049, wherein ABM3 comprises the CDR sequences of CD3-8.
1058. The MBM of embodiment 1048 or embodiment 1049, wherein ABM3 comprises the CDR sequences of CD3-9.
1059. The MBM of embodiment 1048 or embodiment 1049, wherein ABM3 comprises the CDR sequences of CD3-10.
1060. The MBM of embodiment 1048 or embodiment 1049, wherein ABM3 comprises the CDR sequences of CD3-11.
1061. The MBM of embodiment 1048 or embodiment 1049, wherein ABM3 comprises the CDR sequences of CD3-12.
1062. The MBM of embodiment 1048 or embodiment 1049, wherein ABM3 comprises the CDR sequences of CD3-13.
1063. The MBM of embodiment 1048 or embodiment 1049, wherein ABM3 comprises the CDR sequences of CD3-14.
1064. The MBM of embodiment 1048 or embodiment 1049, wherein ABM3 comprises the CDR sequences of CD3-15.
1065. The MBM of embodiment 1048 or embodiment 1049, wherein ABM3 comprises the CDR sequences of CD3-16.
1066. The MBM of embodiment 1048 or embodiment 1049, wherein ABM3 comprises the CDR sequences of CD3-17.
1067. The MBM of embodiment 1048 or embodiment 1049, wherein ABM3 comprises the CDR sequences of CD3-18.
1068. The MBM of embodiment 1048 or embodiment 1049, wherein ABM3 comprises the CDR sequences of CD3-19.
1069. The MBM of embodiment 1048 or embodiment 1049, wherein ABM3 comprises the CDR sequences of CD3-20.
1070. The MBM of embodiment 1048 or embodiment 1049, wherein ABM3 comprises the CDR sequences of CD3-21.
1071. The MBM of embodiment 1048 or embodiment 1049, wherein ABM3 comprises the CDR sequences of CD3-22.
1072. The MBM of embodiment 1048 or embodiment 1049, wherein ABM3 comprises the CDR sequences of CD3-23.
1073. The MBM of embodiment 1048 or embodiment 1049, wherein ABM3 comprises the CDR sequences of CD3-24.
1074. The MBM of embodiment 1048 or embodiment 1049, wherein ABM3 comprises the CDR sequences of CD3-25.
1075. The MBM of embodiment 1048 or embodiment 1049, wherein ABM3 comprises the CDR sequences of CD3-26.
1076. The MBM of embodiment 1048 or embodiment 1049, wherein ABM3 comprises the CDR sequences of CD3-27.
1077. The MBM of embodiment 1048 or embodiment 1049, wherein ABM3 comprises the CDR sequences of CD3-28.
1078. The MBM of embodiment 1048 or embodiment 1049, wherein ABM3 comprises the CDR sequences of CD3-29.
1079. The MBM of embodiment 1048 or embodiment 1049, wherein ABM3 comprises the CDR sequences of CD3-30.
1080. The MBM of embodiment 1048 or embodiment 1049, wherein ABM3 comprises the CDR sequences of CD3-31.
1081. The MBM of embodiment 1048 or embodiment 1049, wherein ABM3 comprises the CDR sequences of CD3-32.
1082. The MBM of embodiment 1048 or embodiment 1049, wherein ABM3 comprises the CDR sequences of CD3-33.
1083. The MBM of embodiment 1048 or embodiment 1049, wherein ABM3 comprises the CDR sequences of CD3-34.
1084. The MBM of embodiment 1048 or embodiment 1049, wherein ABM3 comprises the CDR sequences of CD3-35.
1085. The MBM of embodiment 1048 or embodiment 1049, wherein ABM3 comprises the CDR sequences of CD3-36.
1086. The MBM of embodiment 1048 or embodiment 1049, wherein ABM3 comprises the CDR sequences of CD3-37.
1087. The MBM of embodiment 1048 or embodiment 1049, wherein ABM3 comprises the CDR sequences of CD3-38.
1088. The MBM of embodiment 1048 or embodiment 1049, wherein ABM3 comprises the CDR sequences of CD3-39.
1089. The MBM of embodiment 1048 or embodiment 1049, wherein ABM3 comprises the CDR sequences of CD3-40.
1090. The MBM of embodiment 1048 or embodiment 1049, wherein ABM3 comprises the CDR sequences of CD3-41.
1091. The MBM of embodiment 1048 or embodiment 1049, wherein ABM3 comprises the CDR sequences of CD3-42.
1092. The MBM of embodiment 1048 or embodiment 1049, wherein ABM3 comprises the CDR sequences of CD3-43.
1093. The MBM of embodiment 1048 or embodiment 1049, wherein ABM3 comprises the CDR sequences of CD3-44.
1094. The MBM of embodiment 1048 or embodiment 1049, wherein ABM3 comprises the CDR sequences of CD3-45.
1095. The MBM of embodiment 1048 or embodiment 1049, wherein ABM3 comprises the CDR sequences of CD3-46.
1096. The MBM of embodiment 1048 or embodiment 1049, wherein ABM3 comprises the CDR sequences of CD3-47.
1097. The MBM of embodiment 1048 or embodiment 1049, wherein ABM3 comprises the CDR sequences of CD3-48.
1098. The MBM of embodiment 1048 or embodiment 1049, wherein ABM3 comprises the CDR sequences of CD3-49.
1099. The MBM of embodiment 1048 or embodiment 1049, wherein ABM3 comprises the CDR sequences of CD3-50.
1100. The MBM of embodiment 1048 or embodiment 1049, wherein ABM3 comprises the CDR sequences of CD3-51.
1101. The MBM of embodiment 1048 or embodiment 1049, wherein ABM3 comprises the CDR sequences of CD3-52.
1102. The MBM of embodiment 1048 or embodiment 1049, wherein ABM3 comprises the CDR sequences of CD3-53.
1103. The MBM of embodiment 1048 or embodiment 1049, wherein ABM3 comprises the CDR sequences of CD3-54.
1104. The MBM of embodiment 1048 or embodiment 1049, wherein ABM3 comprises the CDR sequences of CD3-55.
1105. The MBM of embodiment 1048 or embodiment 1049, wherein ABM3 comprises the CDR sequences of CD3-56.
1106. The MBM of embodiment 1048 or embodiment 1049, wherein ABM3 comprises the CDR sequences of CD3-57.
1107. The MBM of embodiment 1048 or embodiment 1049, wherein ABM3 comprises the CDR sequences of CD3-58.
1108. The MBM of embodiment 1048 or embodiment 1049, wherein ABM3 comprises the CDR sequences of CD3-59.
1109. The MBM of embodiment 1048 or embodiment 1049, wherein ABM3 comprises the CDR sequences of CD3-60.
1110. The MBM of embodiment 1048 or embodiment 1049, wherein ABM3 comprises the CDR sequences of CD3-61.
1111. The MBM of embodiment 1048 or embodiment 1049, wherein ABM3 comprises the CDR sequences of CD3-62.
1112. The MBM of embodiment 1048 or embodiment 1049, wherein ABM3 comprises the CDR sequences of CD3-63.
1113. The MBM of embodiment 1048 or embodiment 1049, wherein ABM3 comprises the CDR sequences of CD3-64.
1114. The MBM of embodiment 1048 or embodiment 1049, wherein ABM3 comprises the CDR sequences of CD3-65.
1115. The MBM of embodiment 1048 or embodiment 1049, wherein ABM3 comprises the CDR sequences of CD3-66.
1116. The MBM of embodiment 1048 or embodiment 1049, wherein ABM3 comprises the CDR sequences of CD3-67.
1117. The MBM of embodiment 1048 or embodiment 1049, wherein ABM3 comprises the CDR sequences of CD3-68.
1118. The MBM of embodiment 1048 or embodiment 1049, wherein ABM3 comprises the CDR sequences of CD3-69.
1119. The MBM of embodiment 1048 or embodiment 1049, wherein ABM3 comprises the CDR sequences of CD3-70.
1120. The MBM of embodiment 1048 or embodiment 1049, wherein ABM3 comprises the CDR sequences of CD3-71.
1121. The MBM of embodiment 1048 or embodiment 1049, wherein ABM3 comprises the CDR sequences of CD3-72.
1122. The MBM of embodiment 1048 or embodiment 1049, wherein ABM3 comprises the CDR sequences of CD3-73.
1123. The MBM of embodiment 1048 or embodiment 1049, wherein ABM3 comprises the CDR sequences of CD3-74.
1124. The MBM of embodiment 1048 or embodiment 1049, wherein ABM3 comprises the CDR sequences of CD3-75.
1125. The MBM of embodiment 1048 or embodiment 1049, wherein ABM3 comprises the CDR sequences of CD3-76.
1126. The MBM of embodiment 1048 or embodiment 1049, wherein ABM3 comprises the CDR sequences of CD3-77.
1127. The MBM of embodiment 1048 or embodiment 1049, wherein ABM3 comprises the CDR sequences of CD3-78.
1128. The MBM of embodiment 1048 or embodiment 1049, wherein ABM3 comprises the CDR sequences of CD3-79.
1129. The MBM of embodiment 1048 or embodiment 1049, wherein ABM3 comprises the CDR sequences of CD3-80.
1130. The MBM of embodiment 1048 or embodiment 1049, wherein ABM3 comprises the CDR sequences of CD3-81.
1131. The MBM of embodiment 1048 or embodiment 1049, wherein ABM3 comprises the CDR sequences of CD3-82.
1132. The MBM of embodiment 1048 or embodiment 1049, wherein ABM3 comprises the CDR sequences of CD3-83.
1133. The MBM of embodiment 1048 or embodiment 1049, wherein ABM3 comprises the CDR sequences of CD3-84.
1134. The MBM of embodiment 1048 or embodiment 1049, wherein ABM3 comprises the CDR sequences of CD3-85.
1135. The MBM of embodiment 1048 or embodiment 1049, wherein ABM3 comprises the CDR sequences of CD3-86.
1136. The MBM of embodiment 1048 or embodiment 1049, wherein ABM3 comprises the CDR sequences of CD3-87.
1137. The MBM of embodiment 1048 or embodiment 1049, wherein ABM3 comprises the CDR sequences of CD3-88.
1138. The MBM of embodiment 1048 or embodiment 1049, wherein ABM3 comprises the CDR sequences of CD3-89.
1139. The MBM of embodiment 1048 or embodiment 1049, wherein ABM3 comprises the CDR sequences of CD3-90.
1140. The MBM of embodiment 1048 or embodiment 1049, wherein ABM3 comprises the CDR sequences of CD3-91.
1141. The MBM of embodiment 1048 or embodiment 1049, wherein ABM3 comprises the CDR sequences of CD3-92.
1142. The MBM of embodiment 1048 or embodiment 1049, wherein ABM3 comprises the CDR sequences of CD3-93.
1143. The MBM of embodiment 1048 or embodiment 1049, wherein ABM3 comprises the CDR sequences of CD3-94.
1144. The MBM of embodiment 1048 or embodiment 1049, wherein ABM3 comprises the CDR sequences of CD3-95.
1145. The MBM of embodiment 1048 or embodiment 1049, wherein ABM3 comprises the CDR sequences of CD3-96.
1146. The MBM of embodiment 1048 or embodiment 1049, wherein ABM3 comprises the CDR sequences of CD3-97.
1147. The MBM of embodiment 1048 or embodiment 1049, wherein ABM3 comprises the CDR sequences of CD3-98.
1148. The MBM of embodiment 1048 or embodiment 1049, wherein ABM3 comprises the CDR sequences of CD3-99.
1149. The MBM of embodiment 1048 or embodiment 1049, wherein ABM3 comprises the CDR sequences of CD3-100.
1150. The MBM of embodiment 1048 or embodiment 1049, wherein ABM3 comprises the CDR sequences of CD3-101.
1151. The MBM of embodiment 1048 or embodiment 1049, wherein ABM3 comprises the CDR sequences of CD3-102.
1152. The MBM of embodiment 1048 or embodiment 1049, wherein ABM3 comprises the CDR sequences of CD3-103.
1153. The MBM of embodiment 1048 or embodiment 1049, wherein ABM3 comprises the CDR sequences of CD3-104.
1154. The MBM of embodiment 1048 or embodiment 1049, wherein ABM3 comprises the CDR sequences of CD3-105.
1155. The MBM of embodiment 1048 or embodiment 1049, wherein ABM3 comprises the CDR sequences of CD3-106.
1156. The MBM of embodiment 1048 or embodiment 1049, wherein ABM3 comprises the CDR sequences of CD3-107.
1157. The MBM of embodiment 1048 or embodiment 1049, wherein ABM3 comprises the CDR sequences of CD3-108.
1158. The MBM of embodiment 1048 or embodiment 1049, wherein ABM3 comprises the CDR sequences of CD3-109.
1159. The MBM of embodiment 1048 or embodiment 1049, wherein ABM3 comprises the CDR sequences of CD3-110.
1160. The MBM of embodiment 1048 or embodiment 1049, wherein ABM3 comprises the CDR sequences of CD3-111.
1161. The MBM of embodiment 1048 or embodiment 1049, wherein ABM3 comprises the CDR sequences of CD3-112.
1162. The MBM of embodiment 1048 or embodiment 1049, wherein ABM3 comprises the CDR sequences of CD3-113.
1163. The MBM of embodiment 1048 or embodiment 1049, wherein ABM3 comprises the CDR sequences of CD3-114.
1164. The MBM of embodiment 1048 or embodiment 1049, wherein ABM3 comprises the CDR sequences of CD3-115.
1165. The MBM of embodiment 1048 or embodiment 1049, wherein ABM3 comprises the CDR sequences of CD3-116.
1166. The MBM of embodiment 1048 or embodiment 1049, wherein ABM3 comprises the CDR sequences of CD3-117.
1167. The MBM of embodiment 1048 or embodiment 1049, wherein ABM3 comprises the CDR sequences of CD3-118.
1168. The MBM of embodiment 1048 or embodiment 1049, wherein ABM3 comprises the CDR sequences of CD3-119.
1169. The MBM of embodiment 1048 or embodiment 1049, wherein ABM3 comprises the CDR sequences of CD3-120.
1170. The MBM of embodiment 1048 or embodiment 1049, wherein ABM3 comprises the CDR sequences of CD3-121.
1171. The MBM of embodiment 1048 or embodiment 1049, wherein ABM3 comprises the CDR sequences of CD3-122.
1172. The MBM of embodiment 1048 or embodiment 1049, wherein ABM3 comprises the CDR sequences of CD3-123.
1173. The MBM of embodiment 1048 or embodiment 1049, wherein ABM3 comprises the CDR sequences of CD3-124.
1174. The MBM of embodiment 1048 or embodiment 1049, wherein ABM3 comprises the CDR sequences of CD3-125.
1175. The MBM of embodiment 1048 or embodiment 1049, wherein ABM3 comprises the CDR sequences of CD3-126.
1176. The MBM of embodiment 1048 or embodiment 1049, wherein ABM3 comprises the CDR sequences of CD3-127.
1177. The MBM of embodiment 1048 or embodiment 1049, wherein ABM3 comprises the CDR sequences of CD3-128.
1178. The MBM of any one of embodiments 1050 to 1177, wherein the CDRs are defined by Kabat numbering, as set forth in Table 8B.
1179. The MBM of any one of embodiments 1050 to 1070, wherein the CDRs are defined by Chothia numbering, as set forth in Table 8C.
1180. MBM of any one of embodiments 1050 to 1069, wherein the CDRs are defined by a combination of Kabat and Chothia numbering, as set forth in Table 8D.
1181. The MBM of embodiment 1048 or embodiment 1049, wherein ABM3 comprises the heavy and light chain variable sequences of CD3-1, as set forth in Table 8A.
1182. The MBM of embodiment 1048 or embodiment 1049, wherein ABM3 comprises the heavy and light chain variable sequences of CD3-2, as set forth in Table 8A.
1183. The MBM of embodiment 1048 or embodiment 1049, wherein ABM3 comprises the heavy and light chain variable sequences of CD3-3, as set forth in Table 8A.
1184. The MBM of embodiment 1048 or embodiment 1049, wherein ABM3 comprises the heavy and light chain variable sequences of CD3-4, as set forth in Table 8A.
1185. The MBM of embodiment 1048 or embodiment 1049, wherein ABM3 comprises the heavy and light chain variable sequences of CD3-5, as set forth in Table 8A.
1186. The MBM of embodiment 1048 or embodiment 1049, wherein ABM3 comprises the heavy and light chain variable sequences of CD3-6, as set forth in Table 8A.
1187. The MBM of embodiment 1048 or embodiment 1049, wherein ABM3 comprises the heavy and light chain variable sequences of CD3-7, as set forth in Table 8A.
1188. The MBM of embodiment 1048 or embodiment 1049, wherein ABM3 comprises the heavy and light chain variable sequences of CD3-8, as set forth in Table 8A.
1189. The MBM of embodiment 1048 or embodiment 1049, wherein ABM3 comprises the heavy and light chain variable sequences of CD3-9, as set forth in Table 8A.
1190. The MBM of embodiment 1048 or embodiment 1049, wherein ABM3 comprises the heavy and light chain variable sequences of CD3-10, as set forth in Table 8A.
1191. The MBM of embodiment 1048 or embodiment 1049, wherein ABM3 comprises the heavy and light chain variable sequences of CD3-11, as set forth in Table 8A.
1192. The MBM of embodiment 1048 or embodiment 1049, wherein ABM3 comprises the heavy and light chain variable sequences of CD3-12, as set forth in Table 8A.
1193. The MBM of embodiment 1048 or embodiment 1049, wherein ABM3 comprises the heavy and light chain variable sequences of CD3-13, as set forth in Table 8A.
1194. The MBM of embodiment 1048 or embodiment 1049, wherein ABM3 comprises the heavy and light chain variable sequences of CD3-14, as set forth in Table 8A.
1195. The MBM of embodiment 1048 or embodiment 1049, wherein ABM3 comprises the heavy and light chain variable sequences of CD3-15, as set forth in Table 8A.
1196. The MBM of embodiment 1048 or embodiment 1049, wherein ABM3 comprises the heavy and light chain variable sequences of CD3-16, as set forth in Table 8A.
1197. The MBM of embodiment 1048 or embodiment 1049, wherein ABM3 comprises the heavy and light chain variable sequences of CD3-17, as set forth in Table 8A.
1198. The MBM of embodiment 1048 or embodiment 1049, wherein ABM3 comprises the heavy and light chain variable sequences of CD3-18, as set forth in Table 8A.
1199. The MBM of embodiment 1048 or embodiment 1049, wherein ABM3 comprises the heavy and light chain variable sequences of CD3-19, as set forth in Table 8A.
1200. The MBM of embodiment 1048 or embodiment 1049, wherein ABM3 comprises the heavy and light chain variable sequences of CD3-20, as set forth in Table 8A.
1201. The MBM of embodiment 1048 or embodiment 1049, wherein ABM3 comprises the heavy and light chain variable sequences of CD3-21, as set forth in Table 8A.
1202. The MBM of any one of embodiments 1 to 1047, wherein the component of the TCR complex is TCR-α, TCR-β, or a TCR-α/β dimer.
1203. The MBM of embodiment 1202, wherein ABM3 is an antibody or an antigen-binding domain thereof.
1204. The MBM of embodiment 1203, wherein ABM3 comprises the CDR sequences of BMA031.
1205. The MBM of embodiment 1204, wherein the CDR sequences are defined by Kabat numbering.
1206. The MBM of embodiment 1204, wherein the CDR sequences are defined by Chothia numbering.
1207. The MBM of embodiment 1204, wherein the CDR sequences are defined by a combination of Kabat and Chothia numbering.
1208. The MBM of embodiment 1204, wherein ABM3 comprises the heavy and light chain variable sequences of BMA031.
1209. The MBM of any one of embodiments 1 to 1047, wherein the component of the TCR complex is TCR-γ, TCR-δ, or a TCR-γ/δ dimer.
1210. The MBM of embodiment 1209, wherein ABM3 is an antibody or an antigen-binding domain thereof.
1211. The MBM of embodiment 1210 wherein ABM3 comprises the CDR sequences of δTCS1.
1212. The MBM of embodiment 1211, wherein the CDR sequences are defined by Kabat numbering.
1213. The MBM of embodiment 1211, wherein the CDR sequences are defined by Chothia numbering.
1214. The MBM of embodiment 1211, wherein the CDR sequences are defined by a combination of Kabat and Chothia numbering.
1215. The MBM of embodiment 1211, wherein ABM3 comprises the heavy and light chain variable sequences of δTCS1.
1216. The MBM of any one of embodiments 1 to 1215, wherein ABM3 is an antibody, an antibody fragment, an scFv, a dsFv, a Fv, a Fab, an scFab, a (Fab′)₂, a single domain antibody (SDAB), a VH or VL domain, or a camelid VHH domain.
1217. The MBM of embodiment 1216, wherein ABM3 is an scFv.
1218. The MBM of embodiment 1216, wherein ABM3 is a Fab.
1219. The MBM of any one of embodiments 1 to 1218, which comprises:
   (a) a first monomer or half antibody comprising:
      (i) a first chain comprising a first variant Fc region and a first heavy chain variable domain;
      (ii) a first scFv domain; and
   (b) a second monomer or half antibody comprising:
      (i) a second chain comprising a second variant Fc region and first heavy chain variable domain;
      (ii) a second scFv domain; and
   (c) a third chain comprising a light chain constant domain and a light chain variable domain; wherein:
      (1) the first and second variant Fc regions form a heterodimer,
      (2) the first heavy chain variable domain and the light chain variable domain form ABM1,
      (3) the first scFv domain forms ABM2, and
      (4) the second scFv domain forms ABM3.
1220. The MBM of any one of embodiments 1 to 1218, which comprises:
   (a) a first monomer or half antibody comprising:
      (i) a first chain comprising a first variant Fc region and a first heavy chain variable domain;
      (ii) a first scFv domain; and
   (b) a second monomer or half antibody comprising:
      (i) a second chain comprising a second variant Fc region and first heavy chain variable domain;
      (ii) a second scFv domain; and
   (c) a third chain comprising a light chain constant domain and a light chain variable domain; wherein:
      (1) the first and second variant Fc regions form a heterodimer,
      (2) the first heavy chain variable domain and the light chain variable domain form ABM1,
      (3) the first scFv domain forms ABM3, and
      (4) the second scFv domain forms ABM2.
1221. The MBM of any one of embodiments 1 to 1218, which comprises:
   (a) a first monomer or half antibody comprising:
      (i) a first chain comprising a first variant Fc region and a first heavy chain variable domain;
      (ii) a first scFv domain; and
   (b) a second monomer or half antibody comprising:
      (i) a second chain comprising a second variant Fc region and first heavy chain variable domain;
      (ii) a second scFv domain; and
   (c) a third chain comprising a light chain constant domain and a light chain variable domain; wherein
      (1) the first and second variant Fc regions form a heterodimer,
      (2) the first heavy chain variable domain and the light chain variable domain form ABM2,
      (3) the first scFv domain forms ABM1, and
      (4) the second scFv domain forms ABM3.
1222. The MBM of any one of embodiments 1 to 1218, which comprises:
   (a) a first monomer or half antibody comprising:
      (i) a first chain comprising a first variant Fc region and a first heavy chain variable domain;
      (ii) a first scFv domain; and
   (b) a second monomer or half antibody comprising:
      (i) a second chain comprising a second variant Fc region and first heavy chain variable domain;
      (ii) a second scFv domain; and
   (c) a third chain comprising a light chain constant domain and a light chain variable domain; wherein
      (1) the first and second variant Fc regions form a heterodimer,
      (2) the first heavy chain variable domain and the light chain variable domain form ABM2,
      (3) the first scFv domain forms ABM3, and
      (4) the second scFv domain forms ABM1.
1223. The MBM of any one of embodiments 1 to 1218, which comprises:
   (a) a first monomer or half antibody comprising:
      (i) a first chain comprising a first variant Fc region and a first heavy chain variable domain;
      (ii) a first scFv domain; and
   (b) a second monomer or half antibody comprising:
      (i) a second chain comprising a second variant Fc region and first heavy chain variable domain;
      (ii) a second scFv domain; and
   a third chain comprising a light chain constant domain and a light chain variable domain; wherein:
      (1) the first and second variant Fc regions form a heterodimer,
      (2) the first heavy chain variable domain and the light chain variable domain form ABM3,
      (3) the first scFv domain forms ABM2, and
      (4) the second scFv domain forms ABM1.
1224. The MBM of any one of embodiments 1 to 1218, which comprises:
   (a) a first monomer or half antibody comprising:
      (i) a first chain comprising a first variant Fc region and a first heavy chain variable domain;
      (ii) a first scFv domain; and
   (b) a second monomer or half antibody comprising:
      (i) a second chain comprising a second variant Fc region and first heavy chain variable domain;
      (ii) a second scFv domain; and
   (c) a third chain comprising a light chain constant domain and a light chain variable domain; wherein
      (1) the first and second variant Fc regions form a heterodimer,
      (2) the first heavy chain variable domain and the light chain variable domain form ABM3,
      (3) the first scFv domain forms ABM1, and
      (4) the second scFv domain forms ABM2.
1225. The MBM of any one of embodiments 1219 to 1224, wherein the first and second variant Fc regions comprise the amino acid substitutions S364K/E357Q L368D/K370S.
1226. The MBM of any one of embodiments 1219 to 1224, wherein the first and second variant Fc regions comprise the amino acid substitutions L368D/K370S:S364.
1227. The MBM of any one of embodiments 1219 to 1224, wherein the first and second variant Fc regions comprise the amino acid substitutions L368E/K370S:S364K.
1228. The MBM of any one of embodiments 1219 to 1224, wherein the first and second variant Fc regions comprise the amino acid substitutions T411T/E360E/Q362E:D401K.
1229. The MBM of any one of embodiments 1219 to 1224, wherein the first and second variant Fc regions comprise the amino acid substitutions L368D 370S:S364/E357L.
1230. The MBM of any one of embodiments 1219 to 1224, wherein the first and second variant Fc regions comprise the amino acid substitutions 370S:S364K/E357Q.
1231. The MBM of any one of embodiments 1219 to 1224, wherein the first and second variant Fc regions comprise the amino acid substitutions of any of the steric variants listed in FIG. 4 of WO 2014/110601 (reproduced in Table 2).
1232. The MBM of any one of embodiments 1219 to 1224, wherein the first and second variant Fc regions comprise the amino acid substitutions of any of the variants listed in FIG. 5 of WO 2014/110601 (reproduced in Table 2).
1233. The MBM of any one of embodiments 1219 to 1224, wherein the first and second variant Fc regions comprise the amino acid substitutions of any of the variants listed in FIG. 6 of WO 2014/110601 (reproduced in Table 2).
1234. The MBM of any one of embodiments 1219 to 1233, wherein at least one of the monomers or half antibodies further comprises pl variant substitutions.
1235. The MBM of embodiment 1234 wherein said pl variant substitutions are selected from Table 2.
1236. The MBM of embodiment 1235, wherein the pl variant substitutions comprise the substitutions present in pl_ISO(−).
1237. The MBM of embodiment 1235, wherein the pl variant substitutions comprise the substitutions present in pl_(−)_isosteric_A.
1238. The MBM of embodiment 1235, wherein the pl variant substitutions comprise the substitutions present in pl_(−)_isosteric_B.
1239. The MBM of embodiment 1235, wherein the pl variant substitutions comprise the substitutions present in Pl_ISO(+RR).
1240. The MBM of embodiment 1235, wherein the pl variant substitutions comprise the substitutions present in pl_ISO(+).
1241. The MBM of embodiment 1235, wherein the pl variant substitutions comprise the substitutions present in pl_(+)_isosteric_A.
1242. The MBM of embodiment 1235, wherein the pl variant substitutions comprise the substitutions present in pl_(+)_isosteric_B.
1243. The MBM of embodiment 1235, wherein the pl variant substitutions comprise the substitutions present in pl_(+)_isosteric_E269Q/E272Q.
1244. The MBM of embodiment 1235, wherein the pl variant substitutions comprise the substitutions present in pl_(+)_isosteric_E269Q/E283Q.
1245. The MBM of embodiment 1235, wherein the pl variant substitutions comprise the substitutions present in pl_(+)_isosteric_E2720/E283Q.
1246. The MBM of embodiment 1235, wherein the pl variant substitutions comprise the substitutions present in pl_(+)_isosteric_E269Q.
1247. The MBM of embodiment any of embodiments 1219 to 1246, wherein said first and second scFv domains are covalently attached to the C-terminus of said first and second chains, respectively.
1248. The MBM of embodiment any of embodiments 1219 to 1246, wherein said first and second scFv domains are covalently attached to the N-terminus of said first and second chains, respectively.
1249. The MBM of embodiment any of embodiments 1219 to 1246, wherein each of the scFv domains is attached between said Fc region and the CH domain of said chain.
1250. The MBM of embodiment any of embodiments 1219 to 1249, wherein the scFv domains are covalently attached using one or more domain linkers.
1251. The MBM of embodiment any of embodiments 1219 to 1250, wherein the scFv domains comprise at least one scFv linker.
1252. The MBM of embodiment 1251, wherein at least one scFv linker is charged.
1253. The MBM of embodiment 1252, wherein the charged linker is selected from L1 through L54.
1254. The MBM of embodiment any of embodiments 1219 to 1253, wherein the first and/or second Fc region further comprises one or more amino acid substitution(s) selected from 434A, 434S, 428L, 308F, 259I, 428L/434S, 259I/308F, 436I/428L, 436I or V/434S, 436V/428L, 252Y, 252Y/254T/256E, 259I/308F/428L, 236A, 239D, 239E, 332E, 332D, 239D/332E, 267D, 267E, 328F, 267E/328F, 236A/332E, 239D/332E/330Y, 239D, 332E/330L, 236R, 328R, 236R/328R, 236N/267E, 243L, 298A and 299T.
1255. The MBM of embodiment any of embodiments 1219 to 1253, wherein the first and/or second Fc region further comprises one or more amino acid substitution comprises the amino acid substitution 434A, 434S or 434V.
1256. The MBM of embodiment 1255, wherein the first and/or second Fc region further comprises one or more amino acid substitution comprises the amino acid substitution 428L.
1257. The MBM of any one of embodiments 1255 to 1256, wherein the first and/or second Fc region further comprises one or more amino acid substitution comprises the amino acid substitution 308F.
1258. The MBM of any one of embodiments 1255 to 1257, wherein the first and/or second Fc region further comprises one or more amino acid substitution comprises the amino acid substitution 259I.
1259. The MBM of any one of embodiments 1255 to 1258, wherein the first and/or second Fc region further comprises one or more amino acid substitution comprises the amino acid substitution 436I.
1260. The MBM of any one of embodiments 1255 to 1259, wherein the first and/or second Fc region further comprises one or more amino acid substitution comprises the amino acid substitution 252Y.
1261. The MBM of any one of embodiments 1255 to 1260, wherein the first and/or second Fc region further comprises one or more amino acid substitution comprises the amino acid substitution 254T.
1262. The MBM of any one of embodiments 1255 to 1261, wherein the first and/or second Fc region further comprises one or more amino acid substitution comprises the amino acid substitution 256E.
1263. The MBM of any one of embodiments 1255 to 1262, wherein the first and/or second Fc region further comprises one or more amino acid substitution comprises the amino acid substitution 239D or 239E.
1264. The MBM of any one of embodiments 1255 to 1263, wherein the first and/or second Fc region further comprises one or more amino acid substitution comprises the amino acid substitution 332E or 332D.
1265. The MBM of any one of embodiments 1255 to 1264, wherein the first and/or second Fc region further comprises one or more amino acid substitution comprises the amino acid substitution 267D or 267E.
1266. The MBM of any one of embodiments 1255 to 1265, wherein the first and/or second Fc region further comprises one or more amino acid substitution comprises the amino acid substitution 330L.
1267. The MBM of any one of embodiments 1255 to 1266, wherein the first and/or second Fc region further comprises one or more amino acid substitution comprises the amino acid substitution 236R or 236N.
1268. The MBM of any one of embodiments 1255 to 1267, wherein the first and/or second Fc region further comprises one or more amino acid substitution comprises the amino acid substitution 328R.
1269. The MBM of any one of embodiments 1255 to 1268, wherein the first and/or second Fc region further comprises one or more amino acid substitution comprises the amino acid substitution 243L.
1270. The MBM of any one of embodiments 1255 to 1269, wherein the first and/or second Fc region further comprises one or more amino acid substitution comprises the amino acid substitution 298A.
1271. The MBM of any one of embodiments 1255 to 1270, wherein the first and/or second Fc region further comprises one or more amino acid substitution comprises the amino acid substitution 299T.
1272. The MBM of any one of embodiments 1 to 1271, wherein the component of a human TCR complex is CD3.
1273. The MBM of embodiment 1272, wherein ABM3 is:
   (a) an immunoglobulin scaffold-based ABM which is optionally an anti-CD3 antibody, an antibody fragment, an scFv, a dsFv, a Fv, a Fab, an scFab, a (Fab′)₂, a single domain antibody (SDAB), a VH or VL domain, or a camelid VHH domain; or
   (b) a non-immunoglobulin scaffold-based ABM which is optionally a Kunitz domain, an Adnexin, an Affibody, a DARPin, an Avimer, an Anticalin, a Lipocalin, a Centyrin, a Versabody, a Knottin, an Adnectin, a Pronectin, an Affitin/Nanofitin, an Affilin, an Atrimer/Tetranectin, a bicyclic peptide, a cys-knot, a Fn3 scaffold, an Obody, a Tn3, an Aan Affimer, BD, an Adhiron, a Duocalin, an Alphabody, an Armadillo Repeat Protein, a Repebody, or a Fynomer.
1274. The MBM of embodiment 1272 or embodiment 1273, wherein ABM3 comprises any of the binding sequences set forth in any one of Tables 8A through 8D.
1275. The MBM of embodiment 1274, wherein ABM3 comprises the heavy and light chain CDRs (as defined by Kabat, Chothia or a combination thereof) of any one of the binding domains designated as CD3-1 through CD3-28.
1276. The MBM of embodiment 1275, wherein ABM3 comprises the heavy and light chain CDRs (as defined by Kabat, Chothia or a combination thereof) of the binding domain designated as CD3-1.
1277. The MBM of embodiment 1275, wherein ABM3 comprises the VH and/or VL sequences of the binding domain designated as CD3-1.
1278. The MBM of embodiment 1275, wherein ABM3 comprises the heavy and light chain CDRs (as defined by Kabat, Chothia or a combination thereof) of the binding domain designated as CD3-2.
1279. The MBM of embodiment 1275, wherein ABM3 comprises the VH and/or VL sequences of the binding domain designated as CD3-2.
1280. The MBM of embodiment 1275, wherein ABM3 comprises the heavy and light chain CDRs (as defined by Kabat, Chothia or a combination thereof) of the binding domain designated as CD3-3.
1281. The MBM of embodiment 1275, wherein ABM3 comprises the VH and/or VL sequences of the binding domain designated as CD3-3.
1282. The MBM of embodiment 1275, wherein ABM3 comprises the heavy and light chain CDRs (as defined by Kabat, Chothia or a combination thereof) of the binding domain designated as CD3-4.
1283. The MBM of embodiment 1275, wherein ABM3 comprises the VH and/or VL sequences of the binding domain designated as CD3-4.
1284. The MBM of embodiment 1275, wherein ABM3 comprises the heavy and light chain CDRs (as defined by Kabat, Chothia or a combination thereof) of the binding domain designated as CD3-5.
1285. The MBM of embodiment 1275, wherein ABM3 comprises the VH and/or VL sequences of the binding domain designated as CD3-5.
1286. The MBM of embodiment 1275, wherein ABM3 comprises the heavy and light chain CDRs (as defined by Kabat, Chothia or a combination thereof) of the binding domain designated as CD3-6.
1287. The MBM of embodiment 1275, wherein ABM3 comprises the VH and/or VL sequences of the binding domain designated as CD3-6.
1288. The MBM of embodiment 1275, wherein ABM3 comprises the heavy and light chain CDRs (as defined by Kabat, Chothia or a combination thereof) of the binding domain designated as CD3-7.
1289. The MBM of embodiment 1275, wherein ABM3 comprises the VH and/or VL sequences of the binding domain designated as CD3-7.
1290. The MBM of embodiment 1275, wherein ABM3 comprises the heavy and light chain CDRs (as defined by Kabat, Chothia or a combination thereof) of the binding domain designated as CD3-8.
1291. The MBM of embodiment 1275, wherein ABM3 comprises the VH and/or VL sequences of the binding domain designated as CD3-8.
1292. The MBM of embodiment 1275, wherein ABM3 comprises the heavy and light chain CDRs (as defined by Kabat, Chothia or a combination thereof) of the binding domain designated as CD3-9.
1293. The MBM of embodiment 1275, wherein ABM3 comprises the VH and/or VL sequences of the binding domain designated as CD3-9.
1294. The MBM of embodiment 1275, wherein ABM3 comprises the heavy and light chain CDRs (as defined by Kabat, Chothia or a combination thereof) of the binding domain designated as CD3-10.
1295. The MBM of embodiment 1275, wherein ABM3 comprises the VH and/or VL sequences of the binding domain designated as CD3-10.
1296. The MBM of embodiment 1275, wherein ABM3 comprises the heavy and light chain CDRs (as defined by Kabat, Chothia or a combination thereof) of the binding domain designated as CD3-11.
1297. The MBM of embodiment 1275, wherein ABM3 comprises the VH and/or VL sequences of the binding domain designated as CD3-11.
1298. The MBM of embodiment 1275, wherein ABM3 comprises the heavy and light chain CDRs (as defined by Kabat, Chothia or a combination thereof) of the binding domain designated as CD3-12.
1299. The MBM of embodiment 1275, wherein ABM3 comprises the VH and/or VL sequences of the binding domain designated as CD3-12.
1300. The MBM of embodiment 1275, wherein ABM3 comprises the heavy and light chain CDRs (as defined by Kabat, Chothia or a combination thereof) of the binding domain designated as CD3-13.
1301. The MBM of embodiment 1275, wherein ABM3 comprises the VH and/or VL sequences of the binding domain designated as CD3-13.
1302. The MBM of embodiment 1275, wherein ABM3 comprises the heavy and light chain CDRs (as defined by Kabat, Chothia or a combination thereof) of the binding domain designated as CD3-14.
1303. The MBM of embodiment 1275, wherein ABM3 comprises the VH and/or VL sequences of the binding domain designated as CD3-14.
1304. The MBM of embodiment 1275, wherein ABM3 comprises the heavy and light chain CDRs (as defined by Kabat, Chothia or a combination thereof) of the binding domain designated as CD3-15.
1305. The MBM of embodiment 1275, wherein ABM3 comprises the VH and/or VL sequences of the binding domain designated as CD3-15.
1306. The MBM of embodiment 1275, wherein ABM3 comprises the heavy and light chain CDRs (as defined by Kabat, Chothia or a combination thereof) of the binding domain designated as CD3-16.
1307. The MBM of embodiment 1275, wherein ABM3 comprises the VH and/or VL sequences of the binding domain designated as CD3-16.
1308. The MBM of embodiment 1275, wherein ABM3 comprises the heavy and light chain CDRs (as defined by Kabat, Chothia or a combination thereof) of the binding domain designated as CD3-17.
1309. The MBM of embodiment 1275, wherein ABM3 comprises the VH and/or VL sequences of the binding domain designated as CD3-17.
1310. The MBM of embodiment 1275, wherein ABM3 comprises the heavy and light chain CDRs (as defined by Kabat, Chothia or a combination thereof) of the binding domain designated as CD3-18.
1311. The MBM of embodiment 1275, wherein ABM3 comprises the VH and/or VL sequences of the binding domain designated as CD3-18.
1312. The MBM of embodiment 1275, wherein ABM3 comprises the heavy and light chain CDRs (as defined by Kabat, Chothia or a combination thereof) of the binding domain designated as CD3-19.
1313. The MBM of embodiment 1275, wherein ABM3 comprises the VH and/or VL sequences of the binding domain designated as CD3-19.
1314. The MBM of embodiment 1275, wherein ABM3 comprises the heavy and light chain CDRs (as defined by Kabat, Chothia or a combination thereof) of the binding domain designated as CD3-20.
1315. The MBM of embodiment 1275, wherein ABM3 comprises the VH and/or VL sequences of the binding domain designated as CD3-20.
1316. The MBM of embodiment 1275, wherein ABM3 comprises the heavy and light chain CDRs (as defined by Kabat, Chothia or a combination thereof) of the binding domain designated as CD3-21.
1317. The MBM of embodiment 1275, wherein ABM3 comprises the VH and/or VL sequences of the binding domain designated as CD3-21.
1318. The MBM of embodiment 1275, wherein ABM3 comprises the heavy and light chain CDRs (as defined by Kabat, Chothia or a combination thereof) of the binding domain designated as CD3-22.
1319. The MBM of embodiment 1275, wherein ABM3 comprises the VH and/or VL sequences of the binding domain designated as CD3-22.
1320. The MBM of embodiment 1275, wherein ABM3 comprises the heavy and light chain CDRs (as defined by Kabat, Chothia or a combination thereof) of the binding domain designated as CD3-23.
1321. The MBM of embodiment 1275, wherein ABM3 comprises the VH and/or VL sequences of the binding domain designated as CD3-23.
1322. The MBM of embodiment 1275, wherein ABM3 comprises the heavy and light chain CDRs (as defined by Kabat, Chothia or a combination thereof) of the binding domain designated as CD3-24.
1323. The MBM of embodiment 1275, wherein ABM3 comprises the VH and/or VL sequences of the binding domain designated as CD3-24.
1324. The MBM of embodiment 1275, wherein ABM3 comprises the heavy and light chain CDRs (as defined by Kabat, Chothia or a combination thereof) of the binding domain designated as CD3-25.
1325. The MBM of embodiment 1275, wherein ABM3 comprises the VH and/or VL sequences of the binding domain designated as CD3-25.
1326. The MBM of embodiment 1275, wherein ABM3 comprises the heavy and light chain CDRs (as defined by Kabat, Chothia or a combination thereof) of the binding domain designated as CD3-26.
1327. The MBM of embodiment 1275, wherein ABM3 comprises the VH and/or VL sequences of the binding domain designated as CD3-26.
1328. The MBM of embodiment 1275, wherein ABM3 comprises the heavy and light chain CDRs (as defined by Kabat, Chothia or a combination thereof) of the binding domain designated as CD3-27.
1329. The MBM of embodiment 1275, wherein ABM3 comprises the VH and/or VL sequences of the binding domain designated as CD3-27.
1330. The MBM of embodiment 1275, wherein ABM3 comprises the heavy and light chain CDRs (as defined by Kabat, Chothia or a combination thereof) of the binding domain designated as CD3-28.
1331. The MBM of embodiment 1275, wherein ABM3 comprises the VH and/or VL sequences of the binding domain designated as CD3-28.
1332. The MBM of embodiment 1274, wherein ABM3 comprises the heavy and light chain CDRs (as defined by Kabat) of any one of the binding domains designated as CD3-29 through CD3-128.
1333. The MBM of embodiment 1332, wherein ABM3 comprises the heavy and light chain CDRs (as defined by Kabat) of any one of the binding domains designated as CD3-29 through CD3-38.
1334. The MBM of embodiment 1332, wherein ABM3 comprises the heavy and light chain CDRs (as defined by Kabat) of any one of the binding domains designated as CD3-39 through CD3-48.
1335. The MBM of embodiment 1332, wherein ABM3 comprises the heavy and light chain CDRs (as defined by Kabat) of any one of the binding domains designated as CD3-49 through CD3-58.
1336. The MBM of embodiment 1332, wherein ABM3 comprises the heavy and light chain CDRs (as defined by Kabat) of any one of the binding domains designated as CD3-59 through CD3-68.
1337. The MBM of embodiment 1332, wherein ABM3 comprises the heavy and light chain CDRs (as defined by Kabat) of any one of the binding domains designated as CD3-69 through CD3-78.
1338. The MBM of embodiment 1332, wherein ABM3 comprises the heavy and light chain CDRs (as defined by Kabat) of any one of the binding domains designated as CD3-79 through CD3-88.
1339. The MBM of embodiment 1332, wherein ABM3 comprises the heavy and light chain CDRs (as defined by Kabat) of any one of the binding domains designated as CD3-89 through CD3-98.
1340. The MBM of embodiment 1332, wherein ABM3 comprises the heavy and light chain CDRs (as defined by Kabat) of any one of the binding domains designated as CD3-99 through CD3-108.
1341. The MBM of embodiment 1332, wherein ABM3 comprises the heavy and light chain CDRs (as defined by Kabat) of any one of the binding domains designated as CD3-109 through CD3-118.
1342. The MBM of embodiment 1332, wherein ABM3 comprises the heavy and light chain CDRs (as defined by Kabat) of any one of the binding domains designated as CD3-119 through CD3-128.
1343. The MBM of any one of embodiments 1 to 1271, wherein the component of a human TCR complex is the alpha subunit of the TCR.
1344. The MBM of embodiment 1343, wherein ABM3 is:
   (a) an immunoglobulin scaffold-based ABM which is optionally an antibody, an antibody fragment, an scFv, a dsFv, a Fv, a Fab, an scFab, a (Fab′)₂, a single domain antibody (SDAB), a VH or VL domain, or a camelid VHH domain; or
   (b) a non-immunoglobulin scaffold-based ABM which is optionally a Kunitz domain, an Adnexin, an Affibody, a DARPin, an Avimer, an Anticalin, a Lipocalin, a Centyrin, a Versabody, a Knottin, an Adnectin, a Pronectin, an Affitin/Nanofitin, an Affilin, an Atrimer/Tetranectin, a bicyclic peptide, a cys-knot, a Fn3 scaffold, an Obody, a Tn3, an Aan Affimer, BD, an Adhiron, a Duocalin, an Alphabody, an Armadillo Repeat Protein, a Repebody, or a Fynomer.
1345. The MBM of embodiment 1344, wherein ABM3 comprises CDRs corresponding to the heavy and light chain CDRs of the antibody BMA031.
1346. The MBM of embodiment 1344, wherein ABM3 comprises variable regions corresponding to the VH and VL of the antibody BMA031.
1347. The MBM of any one of embodiments 1 to 1271, wherein the component of a human TCR complex is the beta subunit of the TCR.
1348. The MBM of any one of embodiments 1 to 1271, wherein the component of a human TCR complex is the delta subunit of the TCR.
1349. The MBM of any one of embodiments 1 to 1271, wherein the component of a human TCR complex is the gamma subunit of the TCR.
1350. The MBM of any one of embodiments 1 to 1271, wherein the component of a human TCR complex comprises the alpha and beta subunits of the TCR.
1351. The MBM of any one of embodiments 1 to 1271, wherein the component of a human TCR complex comprises the gamma and delta subunits of the TCR.
1352. The MBM of any one of embodiments 1344 to 1351, wherein ABM3 is an scFv.
1353. The MBM of any one of embodiments 1344 to 1346, wherein ABM3 is a Fab.
1354. The MBM of embodiment 1353, wherein the Fab is a Fab heterodimer.
1355. The MBM of any one of embodiments 1 to 1354 which comprises an Fc domain.
1356. The MBM of embodiment 1355, wherein the Fc domain is an Fc heterodimer.
1357. The MBM of embodiment 1356, wherein the Fc heterodimer comprises any of the Fc modifications set forth in Table 2.
1358. The MBM of embodiment 1356, wherein the Fc heterodimer comprises knob-in-hole (“KIH”) modifications.
1359. The MBM of embodiment 1358, wherein the KIH modifications are any of the KIH modifications described in Section 7.3.1.5.1 or in Table 2.
1360. The MBM of embodiment 1358, wherein the KIH modifications are any of the alternative KIH modifications described in Section 7.3.1.5.2 or in Table 2.
1361. The MBM of any one of embodiments 1356 to 1360, which comprises polar bridge modifications.
1362. The MBM of embodiment 1361, wherein the polar bridge modification are any of the polar bridge modifications described in Section 7.3.1.5.3 or in Table 2.
1363. The MBM of any one of embodiments to 1356 to 1362, which comprises at least one of the Fc modifications designated as Fc 1 through Fc 150.
1364. The MBM of embodiment 1363, which comprises at least one of the Fc modifications designated as Fc 1 through Fc 5.
1365. The MBM of embodiment 1363, which comprises at least one of the Fc modifications designated as Fc 6 through Fc 10.
1366. The MBM of embodiment 1363, which comprises at least one of the Fc modifications designated as Fc 11 through Fc 15.
1367. The MBM of embodiment 1363, which comprises at least one of the Fc modifications designated as Fc 16 through Fc 20.
1368. The MBM of embodiment 1363, which comprises at least one of the Fc modifications designated as Fc 21 through Fc 25.
1369. The MBM of embodiment 1363, which comprises at least one of the Fc modifications designated as Fc 26 through Fc 30.
1370. The MBM of embodiment 1363, which comprises at least one of the Fc modifications designated as Fc 31 through Fc 35.
1371. The MBM of embodiment 1363, which comprises at least one of the Fc modifications designated as Fc 36 through Fc 40.
1372. The MBM of embodiment 1363, which comprises at least one of the Fc modifications designated as Fc 41 through Fc 45.
1373. The MBM of embodiment 1363, which comprises at least one of the Fc modifications designated as Fc 46 through Fc 50.
1374. The MBM of embodiment 1363, which comprises at least one of the Fc modifications designated as Fc 51 through Fc 55.
1375. The MBM of embodiment 1363, which comprises at least one of the Fc modifications designated as Fc 56 through Fc 60.
1376. The MBM of embodiment 1363, which comprises at least one of the Fc modifications designated as Fc 61 through Fc 65.
1377. The MBM of embodiment 1363, which comprises at least one of the Fc modifications designated as Fc 66 through Fc 70.
1378. The MBM of embodiment 1363, which comprises at least one of the Fc modifications designated as Fc 71 through Fc 75.
1379. The MBM of embodiment 1363, which comprises at least one of the Fc modifications designated as Fc 76 through Fc 80.
1380. The MBM of embodiment 1363, which comprises at least one of the Fc modifications designated as Fc 81 through Fc 85.
1381. The MBM of embodiment 1363, which comprises at least one of the Fc modifications designated as Fc 86 through Fc 90.
1382. The MBM of embodiment 1363, which comprises at least one of the Fc modifications designated as Fc 91 through Fc 95.
1383. The MBM of embodiment 1363, which comprises at least one of the Fc modifications designated as Fc 96 through Fc 100.
1384. The MBM of embodiment 1363, which comprises at least one of the Fc modifications designated as Fc 101 through Fc 105.
1385. The MBM of embodiment 1363, which comprises at least one of the Fc modifications designated as Fc 106 through Fc 110.
1386. The MBM of embodiment 1363, which comprises at least one of the Fc modifications designated as Fc 111 through Fc 115.
1387. The MBM of embodiment 1363, which comprises at least one of the Fc modifications designated as Fc 116 through Fc 120.
1388. The MBM of embodiment 1363, which comprises at least one of the Fc modifications designated as Fc 121 through Fc 125.
1389. The MBM of embodiment 1363, which comprises at least one of the Fc modifications designated as Fc 126 through Fc 130.
1390. The MBM of embodiment 1363, which comprises at least one of the Fc modifications designated as Fc 131 through Fc 135.
1391. The MBM of embodiment 1363, which comprises at least one of the Fc modifications designated as Fc 136 through Fc 140.
1392. The MBM of embodiment 1363, which comprises at least one of the Fc modifications designated as Fc 141 through Fc 145.
1393. The MBM of embodiment 1363, which comprises at least one of the Fc modifications designated as Fc 146 through Fc 150.
1394. The MBM of any one of embodiments 1355 to 1393, wherein the Fc domain has altered effector function.
1395. The MBM of embodiment 1394, wherein the Fc domain has altered binding to one or more Fc receptors.
1396. The MBM of embodiment 1395, wherein the one or more Fc receptors comprise FcRN.
1397. The MBM of embodiment 1395 or embodiment 1396, wherein the one or more Fc receptors comprise leukocyte receptors.
1398. The MBM of any one of embodiments 1355 to 1397, wherein the Fc has modified disulfide bond architecture.
1399. The MBM of any one of embodiments 1355 to 1398, wherein the Fc has altered glycosylation patterns.
1400. The MBM of any one of embodiments 1355 to 1399, wherein the Fc comprises a hinge region.
1401. The MBM of embodiment 1400, wherein the hinge region comprises any of the hinge regions described in Section 7.3.2.
1402. The MBM of embodiment 1401, wherein the hinge region comprises the amino acid sequence of the hinge region designated H1.
1403. The MBM of embodiment 1401, wherein the hinge region comprises the amino acid sequence of the hinge region designated H2.
1404. The MBM of embodiment 1401, wherein the hinge region comprises the amino acid sequence of the hinge region designated H3.
1405. The MBM of embodiment 1401, wherein the hinge region comprises the amino acid sequence of the hinge region designated H4.
1406. The MBM of embodiment 1401, wherein the hinge region comprises the amino acid sequence of the hinge region designated H5.
1407. The MBM of embodiment 1401, wherein the hinge region comprises the amino acid sequence of the hinge region designated H6.
1408. The MBM of embodiment 1401, wherein the hinge region comprises the amino acid sequence of the hinge region designated H7.
1409. The MBM of embodiment 1401, wherein the hinge region comprises the amino acid sequence of the hinge region designated H8.
1410. The MBM of embodiment 1401, wherein the hinge region comprises the amino acid sequence of the hinge region designated H9.
1411. The MBM of embodiment 1401, wherein the hinge region comprises the amino acid sequence of the hinge region designated H10.
1412. The MBM of embodiment 1401, wherein the hinge region comprises the amino acid sequence of the hinge region designated H11.
1413. The MBM of embodiment 1401, wherein the hinge region comprises the amino acid sequence of the hinge region designated H12.
1414. The MBM of embodiment 1401, wherein the hinge region comprises the amino acid sequence of the hinge region designated H13.
1415. The MBM of embodiment 1401, wherein the hinge region comprises the amino acid sequence of the hinge region designated H14.
1416. The MBM of embodiment 1401, wherein the hinge region comprises the amino acid sequence of the hinge region designated H15.
1417. The MBM of embodiment 1401, wherein the hinge region comprises the amino acid sequence of the hinge region designated H16.
1418. The MBM of embodiment 1401, wherein the hinge region comprises the amino acid sequence of the hinge region designated H17.
1419. The MBM of embodiment 1401, wherein the hinge region comprises the amino acid sequence of the hinge region designated H18.
1420. The MBM of embodiment 1401, wherein the hinge region comprises the amino acid sequence of the hinge region designated H19.
1421. The MBM of embodiment 1401, wherein the hinge region comprises the amino acid sequence of the hinge region designated H20.
1422. The MBM of embodiment 1401, wherein the hinge region comprises the amino acid sequence of the hinge region designated H21.
1423. The MBM of any one of embodiments 1 to 1422, which comprises at least one scFv domain.
1424. The MBM of embodiment 1423, wherein at least one scFv comprises a linker connecting the VH and VL domains.
1425. The MBM of embodiment 1424, wherein the linker is 5 to 25 amino acids in length.
1426. The MBM of embodiment 1425, wherein the linker is 12 to 20 amino acids in length.
1427. The MBM of any one of embodiments 1424 to 1426, wherein the linker is a charged linker and/or a flexible linker.
1428. The MBM of any one of embodiments 1424 to 1427, wherein the linker is selected from any one of linkers L1 through L54.
1429. The MBM of embodiment 1428, wherein the linker region comprises the amino acid sequence of the linker designated L1.
1430. The MBM of embodiment 1428, wherein the linker region comprises the amino acid sequence of the linker designated L2.
1431. The MBM of embodiment 1428, wherein the linker region comprises the amino acid sequence of the linker designated L3.
1432. The MBM of embodiment 1428, wherein the linker region comprises the amino acid sequence of the linker designated L4.
1433. The MBM of embodiment 1428, wherein the linker region comprises the amino acid sequence of the linker designated L5.
1434. The MBM of embodiment 1428, wherein the linker region comprises the amino acid sequence of the linker designated L6.
1435. The MBM of embodiment 1428, wherein the linker region comprises the amino acid sequence of the linker designated L7.
1436. The MBM of embodiment 1428, wherein the linker region comprises the amino acid sequence of the linker designated L8.
1437. The MBM of embodiment 1428, wherein the linker region comprises the amino acid sequence of the linker designated L9.
1438. The MBM of embodiment 1428, wherein the linker region comprises the amino acid sequence of the linker designated L10.
1439. The MBM of embodiment 1428, wherein the linker region comprises the amino acid sequence of the linker designated L11.
1440. The MBM of embodiment 1428, wherein the linker region comprises the amino acid sequence of the linker designated L12.
1441. The MBM of embodiment 1428, wherein the linker region comprises the amino acid sequence of the linker designated L13.
1442. The MBM of embodiment 1428, wherein the linker region comprises the amino acid sequence of the linker designated L14.
1443. The MBM of embodiment 1428, wherein the linker region comprises the amino acid sequence of the linker designated L15.
1444. The MBM of embodiment 1428, wherein the linker region comprises the amino acid sequence of the linker designated L16.
1445. The MBM of embodiment 1428, wherein the linker region comprises the amino acid sequence of the linker designated L17.
1446. The MBM of embodiment 1428, wherein the linker region comprises the amino acid sequence of the linker designated L18.
1447. The MBM of embodiment 1428, wherein the linker region comprises the amino acid sequence of the linker designated L19.
1448. The MBM of embodiment 1428, wherein the linker region comprises the amino acid sequence of the linker designated L20.
1449. The MBM of embodiment 1428, wherein the linker region comprises the amino acid sequence of the linker designated L21.
1450. The MBM of embodiment 1428, wherein the linker region comprises the amino acid sequence of the linker designated L22.
1451. The MBM of embodiment 1428, wherein the linker region comprises the amino acid sequence of the linker designated L23.
1452. The MBM of embodiment 1428, wherein the linker region comprises the amino acid sequence of the linker designated L24.
1453. The MBM of embodiment 1428, wherein the linker region comprises the amino acid sequence of the linker designated L25.
1454. The MBM of embodiment 1428, wherein the linker region comprises the amino acid sequence of the linker designated L26.
1455. The MBM of embodiment 1428, wherein the linker region comprises the amino acid sequence of the linker designated L27.
1456. The MBM of embodiment 1428, wherein the linker region comprises the amino acid sequence of the linker designated L28.
1457. The MBM of embodiment 1428, wherein the linker region comprises the amino acid sequence of the linker designated L29.
1458. The MBM of embodiment 1428, wherein the linker region comprises the amino acid sequence of the linker designated L30.
1459. The MBM of embodiment 1428, wherein the linker region comprises the amino acid sequence of the linker designated L31.
1460. The MBM of embodiment 1428, wherein the linker region comprises the amino acid sequence of the linker designated L32.
1461. The MBM of embodiment 1428, wherein the linker region comprises the amino acid sequence of the linker designated L33.
1462. The MBM of embodiment 1428, wherein the linker region comprises the amino acid sequence of the linker designated L34.
1463. The MBM of embodiment 1428, wherein the linker region comprises the amino acid sequence of the linker designated L35.
1464. The MBM of embodiment 1428, wherein the linker region comprises the amino acid sequence of the linker designated L36.
1465. The MBM of embodiment 1428, wherein the linker region comprises the amino acid sequence of the linker designated L37.
1466. The MBM of embodiment 1428, wherein the linker region comprises the amino acid sequence of the linker designated L38.
1467. The MBM of embodiment 1428, wherein the linker region comprises the amino acid sequence of the linker designated L39.
1468. The MBM of embodiment 1428, wherein the linker region comprises the amino acid sequence of the linker designated L40.
1469. The MBM of embodiment 1428, wherein the linker region comprises the amino acid sequence of the linker designated L41.
1470. The MBM of embodiment 1428, wherein the linker region comprises the amino acid sequence of the linker designated L42.
1471. The MBM of embodiment 1428, wherein the linker region comprises the amino acid sequence of the linker designated L43.
1472. The MBM of embodiment 1428, wherein the linker region comprises the amino acid sequence of the linker designated L44.
1473. The MBM of embodiment 1428, wherein the linker region comprises the amino acid sequence of the linker designated L45.
1474. The MBM of embodiment 1428, wherein the linker region comprises the amino acid sequence of the linker designated L46.
1475. The MBM of embodiment 1428, wherein the linker region comprises the amino acid sequence of the linker designated L47.
1476. The MBM of embodiment 1428, wherein the linker region comprises the amino acid sequence of the linker designated L48.
1477. The MBM of embodiment 1428, wherein the linker region comprises the amino acid sequence of the linker designated L49.
1478. The MBM of embodiment 1428, wherein the linker region comprises the amino acid sequence of the linker designated L50.
1479. The MBM of embodiment 1428, wherein the linker region comprises the amino acid sequence of the linker designated L51.
1480. The MBM of embodiment 1428, wherein the linker region comprises the amino acid sequence of the linker designated L52.
1481. The MBM of embodiment 1428, wherein the linker region comprises the amino acid sequence of the linker designated L53.
1482. The MBM of embodiment 1428, wherein the linker region comprises the amino acid sequence of the linker designated L54.
1483. The MBM of any one of embodiments 1 to 1482, which comprises at least one Fab domain.
1484. The MBM of embodiment 1483, wherein at least one Fab domain comprises any of the Fab heterodimerization modifications set forth in Table 1.
1485. The MBM of embodiment 1484, wherein at least one Fab domain comprises the Fab heterodimerization modifications designated as F1.
1486. The MBM of embodiment 1484, wherein at least one Fab domain comprises the Fab heterodimerization modifications designated as F2.
1487. The MBM of embodiment 1484, wherein at least one Fab domain comprises the Fab heterodimerization modifications designated as F3.
1488. The MBM of embodiment 1484, wherein at least one Fab domain comprises the Fab heterodimerization modifications designated as F4.
1489. The MBM of embodiment 1484, wherein at least one Fab domain comprises the Fab heterodimerization modifications designated as F5.
1490. The MBM of embodiment 1484, wherein at least one Fab domain comprises the Fab heterodimerization modifications designated as F6.
1491. The MBM of embodiment 1484, wherein at least one Fab domain comprises the Fab heterodimerization modifications designated as F7.
1492. The MBM of any one of embodiments 1 to 1491, which comprises at least two ABMs, an ABM and an ABM chain, or two ABM chains connected to one another via a linker.
1493. The MBM of embodiment 1492, wherein the linker is 5 to 25 amino acids in length.
1494. The MBM of embodiment 1493, wherein the linker is 12 to 20 amino acids in length.
1495. The MBM of any one of embodiments 1492 to 1494, wherein the linker is a charged linker and/or a flexible linker.
1496. The MBM of any one of embodiments 1492 to 1495, wherein the linker is selected from any one of linkers L1 through L54.
1497. The MBM of embodiment 1496, wherein the linker region comprises the amino acid sequence of the linker designated L1.
1498. The MBM of embodiment 1496, wherein the linker region comprises the amino acid sequence of the linker designated L2.
1499. The MBM of embodiment 1496, wherein the linker region comprises the amino acid sequence of the linker designated L3.
1500. The MBM of embodiment 1496, wherein the linker region comprises the amino acid sequence of the linker designated L4.
1501. The MBM of embodiment 1496, wherein the linker region comprises the amino acid sequence of the linker designated L5.
1502. The MBM of embodiment 1496, wherein the linker region comprises the amino acid sequence of the linker designated L6.
1503. The MBM of embodiment 1496, wherein the linker region comprises the amino acid sequence of the linker designated L7.
1504. The MBM of embodiment 1496, wherein the linker region comprises the amino acid sequence of the linker designated L8.
1505. The MBM of embodiment 1496, wherein the linker region comprises the amino acid sequence of the linker designated L9.
1506. The MBM of embodiment 1496, wherein the linker region comprises the amino acid sequence of the linker designated L10.
1507. The MBM of embodiment 1496, wherein the linker region comprises the amino acid sequence of the linker designated L11.
1508. The MBM of embodiment 1496, wherein the linker region comprises the amino acid sequence of the linker designated L12.
1509. The MBM of embodiment 1496, wherein the linker region comprises the amino acid sequence of the linker designated L13.
1510. The MBM of embodiment 1496, wherein the linker region comprises the amino acid sequence of the linker designated L14.
1511. The MBM of embodiment 1496, wherein the linker region comprises the amino acid sequence of the linker designated L15.
1512. The MBM of embodiment 1496, wherein the linker region comprises the amino acid sequence of the linker designated L16.
1513. The MBM of embodiment 1496, wherein the linker region comprises the amino acid sequence of the linker designated L17.
1514. The MBM of embodiment 1496, wherein the linker region comprises the amino acid sequence of the linker designated L18.
1515. The MBM of embodiment 1496, wherein the linker region comprises the amino acid sequence of the linker designated L19.
1516. The MBM of embodiment 1496, wherein the linker region comprises the amino acid sequence of the linker designated L20.
1517. The MBM of embodiment 1496, wherein the linker region comprises the amino acid sequence of the linker designated L21.
1518. The MBM of embodiment 1496, wherein the linker region comprises the amino acid sequence of the linker designated L22.
1519. The MBM of embodiment 1496, wherein the linker region comprises the amino acid sequence of the linker designated L23.
1520. The MBM of embodiment 1496, wherein the linker region comprises the amino acid sequence of the linker designated L24.
1521. The MBM of embodiment 1496, wherein the linker region comprises the amino acid sequence of the linker designated L25.
1522. The MBM of embodiment 1496, wherein the linker region comprises the amino acid sequence of the linker designated L26.
1523. The MBM of embodiment 1496, wherein the linker region comprises the amino acid sequence of the linker designated L27.
1524. The MBM of embodiment 1496, wherein the linker region comprises the amino acid sequence of the linker designated L28.
1525. The MBM of embodiment 1496, wherein the linker region comprises the amino acid sequence of the linker designated L29.
1526. The MBM of embodiment 1496, wherein the linker region comprises the amino acid sequence of the linker designated L30.
1527. The MBM of embodiment 1496, wherein the linker region comprises the amino acid sequence of the linker designated L31.
1528. The MBM of embodiment 1496, wherein the linker region comprises the amino acid sequence of the linker designated L32.
1529. The MBM of embodiment 1496, wherein the linker region comprises the amino acid sequence of the linker designated L33.
1530. The MBM of embodiment 1496, wherein the linker region comprises the amino acid sequence of the linker designated L34.
1531. The MBM of embodiment 1496, wherein the linker region comprises the amino acid sequence of the linker designated L35.
1532. The MBM of embodiment 1496, wherein the linker region comprises the amino acid sequence of the linker designated L36.
1533. The MBM of embodiment 1496, wherein the linker region comprises the amino acid sequence of the linker designated L37.
1534. The MBM of embodiment 1496, wherein the linker region comprises the amino acid sequence of the linker designated L38.
1535. The MBM of embodiment 1496, wherein the linker region comprises the amino acid sequence of the linker designated L39.
1536. The MBM of embodiment 1496, wherein the linker region comprises the amino acid sequence of the linker designated L40.
1537. The MBM of embodiment 1496, wherein the linker region comprises the amino acid sequence of the linker designated L41.
1538. The MBM of embodiment 1496, wherein the linker region comprises the amino acid sequence of the linker designated L42.
1539. The MBM of embodiment 1496, wherein the linker region comprises the amino acid sequence of the linker designated L43.
1540. The MBM of embodiment 1496, wherein the linker region comprises the amino acid sequence of the linker designated L44.
1541. The MBM of embodiment 1496, wherein the linker region comprises the amino acid sequence of the linker designated L45.
1542. The MBM of embodiment 1496, wherein the linker region comprises the amino acid sequence of the linker designated L46.
1543. The MBM of embodiment 1496, wherein the linker region comprises the amino acid sequence of the linker designated L47.
1544. The MBM of embodiment 1496, wherein the linker region comprises the amino acid sequence of the linker designated L48.
1545. The MBM of embodiment 1496, wherein the linker region comprises the amino acid sequence of the linker designated L49.
1546. The MBM of embodiment 1428, wherein the linker region comprises the amino acid sequence of the linker designated L50.
1547. The MBM of embodiment 1428, wherein the linker region comprises the amino acid sequence of the linker designated L51.
1548. The MBM of embodiment 1428, wherein the linker region comprises the amino acid sequence of the linker designated L52.
1549. The MBM of embodiment 1428, wherein the linker region comprises the amino acid sequence of the linker designated L53.
1550. The MBM of embodiment 1496, wherein the linker region comprises the amino acid sequence of the linker designated L54.
1551. The MBM of any one of embodiments 1 to 1550, which is a trivalent MBM.
1552. The MBM of embodiment 1551, wherein the trivalent MBM has any one of the configurations depicted in FIGS. 1B-1O.
1553. The MBM of embodiment 1552, wherein the trivalent MBM has the configuration depicted in FIG. 1B.
1554. The MBM of embodiment 1552, wherein the trivalent MBM has the configuration depicted in FIG. 1C.
1555. The MBM of embodiment 1552, wherein the trivalent MBM has the configuration depicted in FIG. 1D.
1556. The MBM of embodiment 1552, wherein the trivalent MBM has the configuration depicted in FIG. 1E.
1557. The MBM of embodiment 1552, wherein the trivalent MBM has the configuration depicted in FIG. 1F.
1558. The MBM of embodiment 1552, wherein the trivalent MBM has the configuration depicted in FIG. 1G.
1559. The MBM of embodiment 1552, wherein the trivalent MBM has the configuration depicted in FIG. 1H.
1560. The MBM of embodiment 1552, wherein the trivalent MBM has the configuration depicted in FIG. 1I.
1561. The MBM of embodiment 1552, wherein the trivalent MBM has the configuration depicted in FIG. 1J.
1562. The MBM of embodiment 1552, wherein the trivalent MBM has the configuration depicted in FIG. 1K.
1563. The MBM of embodiment 1552, wherein the trivalent MBM has the configuration depicted in FIG. 1L.
1564. The MBM of embodiment 1552, wherein the trivalent MBM has the configuration depicted in FIG. 1M.
1565. The MBM of embodiment 1552, wherein the trivalent MBM has the configuration depicted in FIG. 1N.
1566. The MBM of embodiment 1552, wherein the trivalent MBM has the configuration depicted in FIG. 1O.
1567. The MBM of any one of embodiments 1552 to 1566, in which the ABMs have the configuration designated as T1.
1568. The MBM of any one of embodiments 1552 to 1566, in which the ABMs have the configuration designated as T2.
1569. The MBM of any one of embodiments 1552 to 1566, in which the ABMs have the configuration designated as T3.
1570. The MBM of any one of embodiments 1552 to 1566, in which the ABMs have the configuration designated as T4.
1571. The MBM of any one of embodiments 1552 to 1566, in which the ABMs have the configuration designated as T5.
1572. The MBM of any one of embodiments 1552 to 1566, in which the ABMs have the configuration designated as T6.
1573. The MBM of any one of embodiments 1 to 1550, which is a tetravalent MBM.
1574. The MBM of embodiment 1573, wherein the tetravalent MBM has any one of the configurations depicted in FIGS. 1P-1R.
1575. The MBM of embodiment 1574, wherein the tetravalent MBM has the configuration depicted in FIG. 1P.
1576. The MBM of embodiment 1574, wherein the tetravalent MBM has the configuration depicted in FIG. 1Q.
1577. The MBM of embodiment 1574, wherein the tetravalent MBM has the configuration depicted in FIG. 1R.
1578. The MBM of any one of embodiments 1574 to 1577, in which the ABMs have any of the configurations designated Tv 1 through Tv 24.
1579. The MBM of embodiment 1578, in which the ABMs have the configuration designated Tv 1.
1580. The MBM of embodiment 1578, in which the ABMs have the configuration designated Tv 2.
1581. The MBM of embodiment 1578, in which the ABMs have the configuration designated Tv 3.
1582. The MBM of embodiment 1578, in which the ABMs have the configuration designated Tv 4.
1583. The MBM of embodiment 1578, in which the ABMs have the configuration designated Tv 5.
1584. The MBM of embodiment 1578, in which the ABMs have the configuration designated Tv 6.
1585. The MBM of embodiment 1578, in which the ABMs have the configuration designated Tv 7.
1586. The MBM of embodiment 1578, in which the ABMs have the configuration designated Tv 8.
1587. The MBM of embodiment 1578, in which the ABMs have the configuration designated Tv 9.
1588. The MBM of embodiment 1578, in which the ABMs have the configuration designated Tv 10.
1589. The MBM of embodiment 1578, in which the ABMs have the configuration designated Tv 11.
1590. The MBM of embodiment 1578, in which the ABMs have the configuration designated Tv 12.
1591. The MBM of embodiment 1578, in which the ABMs have the configuration designated Tv 13.
1592. The MBM of embodiment 1578, in which the ABMs have the configuration designated Tv 14.
1593. The MBM of embodiment 1578, in which the ABMs have the configuration designated Tv 15.
1594. The MBM of embodiment 1578, in which the ABMs have the configuration designated Tv 16.
1595. The MBM of embodiment 1578, in which the ABMs have the configuration designated Tv 17.
1596. The MBM of embodiment 1578, in which the ABMs have the configuration designated Tv 18.
1597. The MBM of embodiment 1578, in which the ABMs have the configuration designated Tv 19.
1598. The MBM of embodiment 1578, in which the ABMs have the configuration designated Tv 20.
1599. The MBM of embodiment 1578, in which the ABMs have the configuration designated Tv 21.
1600. The MBM of embodiment 1578, in which the ABMs have the configuration designated Tv 22.
1601. The MBM of embodiment 1578, in which the ABMs have the configuration designated Tv 23.
1602. The MBM of embodiment 1578, in which the ABMs have the configuration designated Tv 24
1603. The MBM of any one of embodiments 1 to 1550, which is a pentavalent MBM.
1604. The MBM of embodiment 1603, wherein the pentavalent MBM has the configuration depicted in FIG. 1S.
1605. The MBM of embodiment 1604, in which the ABMs have any of the configurations designated Pv 1 through Pv 80.
1606. The MBM of embodiment 1605, in which the ABMs have a configuration selected from any one of the configurations designated Pv 1 through Pv 10.
1607. The MBM of embodiment 1605, in which the ABMs have a configuration selected from any one of the configurations designated Pv 11 through Pv 20.
1608. The MBM of embodiment 1605, in which the ABMs have a configuration selected from any one of the configurations designated Pv 21 through Pv 30.
1609. The MBM of embodiment 1605, in which the ABMs have a configuration selected from any one of the configurations designated Pv 31 through Pv 40.
1610. The MBM of embodiment 1605, in which the ABMs have a configuration selected from any one of the configurations designated Pv 41 through Pv 50.
1611. The MBM of embodiment 1605, in which the ABMs have a configuration selected from any one of the configurations designated Pv 51 through Pv 60.
1612. The MBM of embodiment 1605, in which the ABMs have a configuration selected from any one of the configurations designated Pv 61 through Pv 70.
1613. The MBM of embodiment 1605, in which the ABMs have a configuration selected from any one of the configurations designated Pv 71 through Pv 80.
1614. The MBM of embodiment 1605, in which the ABMs have a configuration selected from any one of the configurations designated Pv 81 through Pv 90.
1615. The MBM of embodiment 1605, in which the ABMs have a configuration selected from any one of the configurations designated Pv 91 through Pv 100.
1616. The MBM of any one of embodiments 1 to 1550, which is a hexavalent MBM.
1617. The MBM of embodiment 1616, wherein the hexavalent MBM has the configuration depicted in FIG. 1T or FIG. 1U.
1618. The MBM of embodiment 1617, wherein the hexavalent MBM has the configuration depicted in FIG. 1T.
1619. The MBM of embodiment 1617, wherein the hexavalent MBM has the configuration depicted in FIG. 1U.
1620. The MBM of any one of embodiments 1617 to 1619, in which the ABMs have any of the configurations designated Hv 1 through Hv 330.
1621. The MBM of embodiment 1620, in which the ABMs have a configuration selected from any one of the configurations designated Hv 1 through Hv 10.
1622. The MBM of embodiment 1620, in which the ABMs have a configuration selected from any one of the configurations designated Hv 11 through Hv 20.
1623. The MBM of embodiment 1620, in which the ABMs have a configuration selected from any one of the configurations designated Hv 21 through Hv 30.
1624. The MBM of embodiment 1620, in which the ABMs have a configuration selected from any one of the configurations designated Hv 31 through Hv 40.
1625. The MBM of embodiment 1620, in which the ABMs have a configuration selected from any one of the configurations designated Hv 41 through Hv 50.
1626. The MBM of embodiment 1620, in which the ABMs have a configuration selected from any one of the configurations designated Hv 51 through Hv 60.
1627. The MBM of embodiment 1620, in which the ABMs have a configuration selected from any one of the configurations designated Hv 61 through Hv 70.
1628. The MBM of embodiment 1620, in which the ABMs have a configuration selected from any one of the configurations designated Hv 71 through Hv 80.
1629. The MBM of embodiment 1620, in which the ABMs have a configuration selected from any one of the configurations designated Hv 81 through Hv 90.
1630. The MBM of embodiment 1620, in which the ABMs have a configuration selected from any one of the configurations designated Hv 91 through Hv 100.
1631. The MBM of embodiment 1620, in which the ABMs have a configuration selected from any one of the configurations designated Hv 101 through Hv 110.
1632. The MBM of embodiment 1620, in which the ABMs have a configuration selected from any one of the configurations designated Hv 111 through Hv 120.
1633. The MBM of embodiment 1620, in which the ABMs have a configuration selected from any one of the configurations designated Hv 121 through Hv 130.
1634. The MBM of embodiment 1620, in which the ABMs have a configuration selected from any one of the configurations designated Hv 131 through Hv 140.
1635. The MBM of embodiment 1620, in which the ABMs have a configuration selected from any one of the configurations designated Hv 141 through Hv 150.
1636. The MBM of embodiment 1620, in which the ABMs have a configuration selected from any one of the configurations designated Hv 151 through Hv 160.
1637. The MBM of embodiment 1620, in which the ABMs have a configuration selected from any one of the configurations designated Hv 161 through Hv 70.
1638. The MBM of embodiment 1620, in which the ABMs have a configuration selected from any one of the configurations designated Hv 171 through Hv 80.
1639. The MBM of embodiment 1620, in which the ABMs have a configuration selected from any one of the configurations designated Hv 181 through Hv 90.
1640. The MBM of embodiment 1620, in which the ABMs have a configuration selected from any one of the configurations designated Hv 191 through Hv 200.
1641. The MBM of embodiment 1620, in which the ABMs have a configuration selected from any one of the configurations designated Hv 201 through Hv 210.
1642. The MBM of embodiment 1620, in which the ABMs have a configuration selected from any one of the configurations designated Hv 211 through Hv 220.
1643. The MBM of embodiment 1620, in which the ABMs have a configuration selected from any one of the configurations designated Hv 221 through Hv 230.
1644. The MBM of embodiment 1620, in which the ABMs have a configuration selected from any one of the configurations designated Hv 231 through Hv 240.
1645. The MBM of embodiment 1620, in which the ABMs have a configuration selected from any one of the configurations designated Hv 241 through Hv 250.
1646. The MBM of embodiment 1620, in which the ABMs have a configuration selected from any one of the configurations designated Hv 251 through Hv 260.
1647. The MBM of embodiment 1620, in which the ABMs have a configuration selected from any one of the configurations designated Hv 261 through Hv 270.
1648. The MBM of embodiment 1620, in which the ABMs have a configuration selected from any one of the configurations designated Hv 271 through Hv 280.
1649. The MBM of embodiment 1620, in which the ABMs have a configuration selected from any one of the configurations designated Hv 281 through Hv 290.
1650. The MBM of embodiment 1620, in which the ABMs have a configuration selected from any one of the configurations designated Hv 291 through Hv 300.
1651. The MBM of embodiment 1620, in which the ABMs have a configuration selected from any one of the configurations designated Hv 301 through Hv 310.
1652. The MBM of embodiment 1620, in which the ABMs have a configuration selected from any one of the configurations designated Hv 311 through Hv 320.
1653. The MBM of embodiment 1620, in which the ABMs have a configuration selected from any one of the configurations designated Hv 321 through Hv 330.
1654. The MBM of any one of embodiments 1 to 1653, wherein any one, any two, or all three of ABM1, ABM2 and ABM3 has cross-species reactivity.
1655. The MBM of embodiment 1654, wherein ABM1 further binds specifically to TAA 1 in one or more non-human mammalian species.
1656. The MBM of embodiment 1654 or embodiment 1655, wherein ABM2 further binds specifically to TAA 2 in one or more non-human mammalian species.
1657. The MBM of any one of embodiments 1654 to 1656, wherein ABM3 further binds specifically to the component of the TCR complex in one or more non-human mammalian species.
1658. The MBM of any one of embodiments 1654 to 1657, wherein the one or more non-human mammalian species comprises one or more non-human primate species.
1659. The MBM of embodiment 1658, wherein the one or more non-human primate species comprises Macaca fascicularis.
1660. The MBM of embodiment 1658, wherein the one or more non-human primate species comprises Macaca mulatta.
1661. The MBM of embodiment 1658, wherein the one or more non-human primate species comprises Macaca nemestrina.
1662. The MBM of any one of embodiments 1654 to 1661, wherein the one or more non-human mammalian species comprises Mus musculus.
1663. The MBM of any one of embodiments 1 to 1662, wherein any one, any two, or all three of ABM1, ABM2 and ABM3 does not have cross-species reactivity.
1664. The MBM of any one of embodiments 1 to 1663, wherein the MBM is a trispecific binding molecule (TBM).
1665. A conjugate comprising the MBM of any one of embodiments 1 to 1664 and an agent, optionally a therapeutic agent, a diagnostic agent, a masking moiety, a cleavable moiety, or any combination thereof.
1666. The conjugate of embodiment 1665, wherein the agent is a cytotoxic or cytostatic agent.
1667. The conjugate of embodiment 1666, wherein the agent is any of the agents described in Section 7.8.
1668. The conjugate of embodiment 1666 or 1667, wherein the agent is any of the agents described in Section 7.8.1.
1669. The conjugate of any one of embodiments 1665 to 1668, wherein the MBM is conjugated to a radionuclide.
1670. The conjugate of any one of embodiments 1665 to 1668, wherein the MBM is conjugated to an alkylating agent.
1671. The conjugate of any one of embodiments 1665 to 1668, wherein the MBM is conjugated to a topoisomerase inhibitor, which is optionally a topoisomerase I inhibitor or a topoisomerase II inhibitor.
1672. The conjugate of any one of embodiments 1665 to 1668, wherein the MBM is conjugated to a DNA damaging agent.
1673. The conjugate of any one of embodiments 1665 to 1668, wherein the MBM is conjugated to a DNA intercalating agent, optionally a groove binding agent such as a minor groove binding agent.
1674. The conjugate of any one of embodiments 1665 to 1668, wherein the MBM is conjugated to a RNA/DNA antimetabolite.
1675. The conjugate of any one of embodiments 1665 to 1668, wherein the MBM is conjugated to a kinase inhibitor.
1676. The conjugate of any one of embodiments 1665 to 1668, wherein the MBM is conjugated to a protein synthesis inhibitor.
1677. The conjugate of any one of embodiments 1665 to 1668, wherein the MBM is conjugated to a histone deacetylase (HDAC) inhibitor.
1678. The conjugate of any one of embodiments 1665 to 1668, wherein the MBM is conjugated to a mitochondrial. Inhibitor, which is optionally an inhibitor of a phosphoryl transfer reaction in mitochondria.
1679. The conjugate of any one of embodiments 1665 to 1668, wherein the MBM is conjugated to an antimitotic agent.
1680. The conjugate of any one of embodiments 1665 to 1668, wherein the MBM is conjugated to a maytansinoid.
1681. The conjugate of any one of embodiments 1665 to 1668, wherein the MBM is conjugated to a kinesin inhibitor.
1682. The conjugate of any one of embodiments 1665 to 1668, wherein the MBM is conjugated to a kinesin-like protein KIF11 inhibitor.
1683. The conjugate of any one of embodiments 1665 to 1668, wherein the MBM is conjugated to a V-ATPase (vacuolar-type H+-ATPase) inhibitor.
1684. The conjugate of any one of embodiments 1665 to 1668, wherein the MBM is conjugated to a pro-apoptotic agent.
1685. The conjugate of any one of embodiments 1665 to 1668, wherein the MBM is conjugated to a Bcl2 (B-cell lymphoma 2) inhibitor.
1686. The conjugate of any one of embodiments 1665 to 1668, wherein the MBM is conjugated to an MCL1 (myeloid cell leukemia 1) inhibitor.
1687. The conjugate of any one of embodiments 1665 to 1668, wherein the MBM is conjugated to a HSP90 (heat shock protein 90) inhibitor.
1688. The conjugate of any one of embodiments 1665 to 1668, wherein the MBM is conjugated to an IAP (inhibitor of apoptosis) inhibitor.
1689. The conjugate of any one of embodiments 1665 to 1668, wherein the MBM is conjugated to an mTOR (mechanistic target of rapamycin) inhibitor.
1690. The conjugate of any one of embodiments 1665 to 1668, wherein the MBM is conjugated to a microtubule stabilizer.
1691. The conjugate of any one of embodiments 1665 to 1668, wherein the MBM is conjugated to a microtubule destabilizer.
1692. The conjugate of any one of embodiments 1665 to 1668, wherein the MBM is conjugated to an auristatin.
1693. The conjugate of any one of embodiments 1665 to 1668, wherein the MBM is conjugated to a dolastatin.
1694. The conjugate of any one of embodiments 1665 to 1668, wherein the MBM is conjugated to a MetAP (methionine aminopeptidase).
1695. The conjugate of any one of embodiments 1665 to 1668, wherein the MBM is conjugated to a CRM1 (chromosomal maintenance 1) inhibitor.
1696. The conjugate of any one of embodiments 1665 to 1668, wherein the MBM is conjugated to a DPPIV (dipeptidyl peptidase IV) inhibitor.
1697. The conjugate of any one of embodiments 1665 to 1668, wherein the MBM is conjugated to a proteasome inhibitor.
1698. The conjugate of any one of embodiments 1665 to 1668, wherein the MBM is conjugated to a protein synthesis inhibitor.
1699. The conjugate of any one of embodiments 1665 to 1668, wherein the MBM is conjugated to a CDK2 (cyclin-dependent kinase 2) inhibitor.
1700. The conjugate of any one of embodiments 1665 to 1668, wherein the MBM is conjugated to a CDK9 (cyclin-dependent kinase 9) inhibitor.
1701. The conjugate of any one of embodiments 1665 to 1668, wherein the MBM is conjugated to a RNA polymerase inhibitor.
1702. The conjugate of any one of embodiments 1665 to 1668, wherein the MBM is conjugated to a DHFR (dihydrofolate reductase) inhibitor.
1703. The conjugate of any one of embodiments 1665 to 1702, wherein the agent is attached to the MBM with a linker, which is optionally a cleavable linker or a non-cleavable linker, e.g., a linker as described in Section 7.8.2.
1704. The conjugate of any one of embodiments 1666 to 1703, wherein the cytotoxic or cytostatic agent is conjugated to the MBM via a linker as described in Section 7.8.2.
1705. A pharmaceutical composition comprising the MBM of any one of embodiments 1 to 1664 or the conjugate of any one of embodiments 1665 to 1704 and an excipient.
1706. A method of treating a subject with a B cell malignancy, comprising administering to a subject suffering from the B cell malignancy an effective amount of the MBM of any one of embodiments 1 to 1664, the conjugate of any one of embodiments 1665 to 1704, or the pharmaceutical composition of embodiment 1705.
1707. The method of embodiment 1706, wherein the B cell malignancy comprises cancerous B cells expressing both TAA 1 and TAA 2.
1708. The method of embodiment 1706, wherein the B cell malignancy comprises cancerous B cells expressing TAA 1, but not TAA 2, and cancerous B cells expressing TAA 2, but not TAA 1.
1709. The method of any one of embodiments 1706 to 1708, wherein the B cell malignancy is selected from selected from Hodgkin's lymphoma, non-Hodgkin's lymphoma and multiple myeloma.
1710. The method of embodiment 1709, wherein the B cell malignancy is Hodgkin's lymphoma.
1711. The method of embodiment 1709, wherein the B cell malignancy is non-Hodgkin's lymphoma.
1712. The method of embodiment 1711, wherein the non-Hodgkin's lymphoma is diffuse large B-cell lymphoma (DLBCL), follicular lymphoma, chronic lymphocytic leukemia (CLL)/small lymphocytic lymphoma (SLL), mantle cell lymphoma (MCL), marginal zone lymphoma, Burkitt lymphoma, lymphoplasmacytic lymphoma (Waldenstrom macroglobulinemia), hairy cell leukemia, or primary central nervous system (CNS) lymphoma.
1713. The method of embodiment 1712, wherein the non-Hodgkin's lymphoma is diffuse large B-cell lymphoma (DLBCL).
1714. The method of embodiment 1712, wherein the non-Hodgkin's lymphoma is follicular lymphoma.
1715. The method of embodiment 1712, wherein the non-Hodgkin's lymphoma is chronic lymphocytic leukemia (CLL)/small lymphocytic lymphoma (SLL).
1716. The method of embodiment 1712, wherein the non-Hodgkin's lymphoma is mantle cell lymphoma (MCL).
1717. The method of embodiment 1712, wherein the non-Hodgkin's lymphoma is marginal zone lymphoma.
1718. The method of embodiment 1712, wherein the non-Hodgkin's lymphoma is Burkitt lymphoma.
1719. The method of embodiment 1712, wherein the non-Hodgkin's lymphoma is lymphoplasmacytic lymphoma (Waldenstrom macroglobulinemia).
1720. The method of embodiment 1712, wherein the non-Hodgkin's lymphoma is hairy cell leukemia.
1721. The method of embodiment 1712, wherein the non-Hodgkin's lymphoma is primary central nervous system (CNS) lymphoma.
1722. The method of embodiment 1712, wherein the non-Hodgkin's lymphoma is primary mediastinal large B-cell lymphoma.
1723. The method of embodiment 1712, wherein the non-Hodgkin's lymphoma is mediastinal grey-zone lymphoma (MGZL).
1724. The method of embodiment 1712, wherein the non-Hodgkin's lymphoma is splenic marginal zone B-cell lymphoma.
1725. The method of embodiment 1712, wherein the non-Hodgkin's lymphoma is extranodal marginal zone B-cell lymphoma of MALT.
1726. The method of embodiment 1712, wherein the non-Hodgkin's lymphoma is nodal marginal zone B-cell lymphoma.
1727. The method of embodiment 1712, wherein the non-Hodgkin's lymphoma is primary effusion lymphoma.
1728. The method of embodiment 1709, wherein the B cell malignancy is multiple myeloma.
1729. The method of embodiment 1709, wherein the B cell malignancy is a plasmacytic dendritic cell neoplasm.
1730. The method of any one of embodiments 1706 to 1729, further comprising administering at least one additional agent to the subject.
1731. The method of embodiment 1730, wherein the additional agent is a chemotherapeutic agent.
1732. The method of embodiment 1730 or embodiment 1731, wherein the additional agent is an anthracycline.
1733. The method of embodiment 1730 or embodiment 1731, wherein the additional agent is a vinca alkaloid.
1734. The method of embodiment 1730 or embodiment 1731, wherein the additional agent is an alkylating agent.
1735. The method of embodiment 1730 or embodiment 1731, wherein the additional agent is an immune cell antibody.
1736. The method of embodiment 1730 or embodiment 1731, wherein the additional agent is an antimetabolite.
1737. The method of embodiment 1730 or embodiment 1731, wherein the additional agent is an adenosine deaminase inhibitor 1738. The method of embodiment 1730 or embodiment 1731, wherein the additional agent is an mTOR inhibitor.
1739. The method of embodiment 1730 or embodiment 1731, wherein the additional agent is a TNFR glucocorticoid induced TNFR related protein (GITR) agonist.
1740. The method of embodiment 1730 or embodiment 1731, wherein the additional agent is a proteasome inhibitor.
1741. The method of embodiment 1730 or embodiment 1731, wherein the additional agent is a BH3 mimetic.
1742. The method of embodiment 1730 or embodiment 1731, wherein the additional agent is a cytokine.
1743. The method of embodiment 1730 or embodiment 1731, wherein the additional agent prevents or slows shedding of TAA1 and/or TAA2 from a cancer cell.
1744. The method of embodiment 1743, wherein the additional agent comprises an ADAM10 inhibitor and/or an ADAM 17 inhibitor.
1745. The method of embodiment 1743, wherein the additional agent comprises a phospholipase inhibitor.
1746. The method of embodiment 1730 or embodiment 1731, wherein the additional agent is a gamma secretase inhibitor.
1747. The method of embodiment 1730 or embodiment 1731, wherein the additional agent is an immunomodulatory.
1748. The method of embodiment 1730 or embodiment 1731, wherein the additional agent is a thalidomide derivative.
1749. The method of any one of embodiments 1730 to 1748, wherein the additional agent is not an antibody.
1750. A method of treating a subject with an autoimmune disorder, comprising administering to a subject suffering from the autoimmune disorder an effective amount of the MBM of any one of embodiments 1 to 1664, the conjugate of any one of embodiments 1665 to
1704, or the pharmaceutical composition of embodiment 1705.
1751. The method of embodiment 1750, wherein the autoimmune disorder is selected from systemic lupus erythematosus (SLE), Sjögren's syndrome, scleroderma, rheumatoid arthritis (RA), juvenile idiopathic arthritis, graft versus host disease, dermatomyositis, type I diabetes mellitus, Hashimoto's thyroiditis, Graves's disease, Addison's disease, celiac disease, Crohn's Disease, pernicious anaemia, pemphigus vulgaris, vitiligo, autoimmune haemolytic anaemia, idiopathic thrombocytopenic purpura, giant cell arteritis, myasthenia gravis, multiple sclerosis (MS) (e.g., relapsing-remitting MS (RRMS)), glomerulonephritis, Goodpasture's syndrome, bullous pemphigoid, colitis ulcerosa, Guillain-Barré syndrome, chronic inflammatory demyelinating polyneuropathy, anti-phospholipid syndrome, narcolepsy, sarcoidosis, and Wegener's granulomatosis.
1752. The method of embodiment 1751, wherein the autoimmune disorder is systemic lupus erythematosus (SLE).
1753. The method of embodiment 1751, wherein the autoimmune disorder is Sjögren's syndrome.
1754. The method of embodiment 1751, wherein the autoimmune disorder is scleroderma.
1755. The method of embodiment 1751, wherein the autoimmune disorder is rheumatoid arthritis (RA).
1756. The method of embodiment 1751, wherein the autoimmune disorder is juvenile idiopathic arthritis.
1757. The method of embodiment 1751, wherein the autoimmune disorder is graft versus host disease.
1758. The method of embodiment 1751, wherein the autoimmune disorder is dermatomyositis.
1759. The method of embodiment 1751, wherein the autoimmune disorder is type I diabetes mellitus.
1760. The method of embodiment 1751, wherein the autoimmune disorder is Hashimoto's thyroiditis.
1761. The method of embodiment 1751, wherein the autoimmune disorder is Graves's disease.
1762. The method of embodiment 1751, wherein the autoimmune disorder is Addison's disease.
1763. The method of embodiment 1751, wherein the autoimmune disorder is celiac disease.
1764. The method of embodiment 1751, wherein the autoimmune disorder is Crohn's Disease.
1765. The method of embodiment 1751, wherein the autoimmune disorder is pernicious anaemia.
1766. The method of embodiment 1751, wherein the autoimmune disorder is pemphigus vulgaris.
1767. The method of embodiment 1751, wherein the autoimmune disorder is vitiligo.
1768. The method of embodiment 1751, wherein the autoimmune disorder is autoimmune haemolytic anaemia.
1769. The method of embodiment 1751, wherein the autoimmune disorder is idiopathic thrombocytopenic purpura.
1770. The method of embodiment 1751, wherein the autoimmune disorder is giant cell arteritis.
1771. The method of embodiment 1751, wherein the autoimmune disorder is myasthenia gravis.
1772. The method of embodiment 1751, wherein the autoimmune disorder is multiple sclerosis (MS).
1773. The method of embodiment 1772, wherein the MS is relapsing-remitting MS (RRMS).
1774. The method of embodiment 1751, wherein the autoimmune disorder is glomerulonephritis.
1775. The method of embodiment 1751, wherein the autoimmune disorder is Goodpasture's syndrome.
1776. The method of embodiment 1751, wherein the autoimmune disorder is bullous pemphigoid.
1777. The method of embodiment 1751, wherein the autoimmune disorder is colitis ulcerosa.
1778. The method of embodiment 1751, wherein the autoimmune disorder is Guillain-Barré syndrome.
1779. The method of embodiment 1751, wherein the autoimmune disorder is chronic inflammatory demyelinating polyneuropathy.
1780. The method of embodiment 1751, wherein the autoimmune disorder is anti-phospholipid syndrome.
1781. The method of embodiment 1751, wherein the autoimmune disorder is narcolepsy.
1782. The method of embodiment 1751, wherein the autoimmune disorder is sarcoidosis.
1783. The method of embodiment 1751, wherein the autoimmune disorder is Wegener's granulomatosis.
1784. A nucleic acid or plurality of nucleic acids encoding the MBM of any one of embodiments 1 to 1664.
1785. The nucleic acid or plurality of nucleic acids of embodiment 1784 which is a DNA (are DNAs).
1786. The nucleic acid or plurality of nucleic acids of embodiment 1785 which are in the form of one or more vectors, optionally expression vectors.
1787. The nucleic acid or plurality of nucleic acids of embodiment 1784 which is a mRNA (are mRNAs).
1788. A cell engineered to express the MBM of any one of embodiments 1 to 1664.
1789. A cell transfected with one or more expression vectors comprising one or more nucleic acid sequences encoding the MBM of any one of embodiments 1 to 1664 under the control of one or more promoters.
1790. The cell of embodiment 1788 or embodiment 1789, wherein expression of the MBM is under the control of one or more inducible promoters.
1791. The cell of any one of embodiments 1788 to 1790, wherein the MBM is produced in secretable form.
1792. A method of producing a MBM, comprising:
   (a) culturing the cell of any one of embodiments 1788 to 1791 in conditions under which the MBM is expressed; and
   (b) recovering the MBM from the cell culture.

1. A multispecific binding molecule (MBM), comprising:
(a) an antigen-binding module 1 (ABM1) that binds specifically to a first human tumor-associated antigen that is expressed on cancerous B cells (TAA 1);
(b) an antigen-binding module 2 (ABM2) that binds specifically to a second human tumor-associated antigen that is expressed on cancerous B cells (TAA 2), and
(c) an antigen-binding module 3 (ABM3) that binds specifically to a component of a human T-cell receptor (TCR) complex.

2. The MBM of claim 1, wherein TAA 1 or TAA 2 or both are expressed on cancerous B cells that are B cell-derived plasma cells.

3. (canceled)

4. The MBM of claim 1, wherein TAA 1 or TAA 2 or both are expressed on cancerous B cells that are not plasma cells.

5. (canceled)

6. The MBM of claim 1, wherein TAA 1 and TAA 2 are expressed on the same cancerous B cell.

7. The MBM of claim 1, wherein TAA 1 and TAA 2 are expressed on different cancerous B cells.

8. (canceled)

9. The MBM of claim 1, wherein TAA 1 and TAA 2 are each independently CD19, CD20, CD22, CD123, BCMA, CD33, CLL1, CD138, CS1, CD38, CD133, FLT3, CD52, TNFRSF13C, TNFRSF13B, CXCR4, PD-L1, LY9, CD200, FCGR2B, CD21, CD23, CD24, CD40L, CD72, CD79a, or CD79b.

10. (canceled)

11. The MBM of claim 1, wherein ABM1, ABM2, or both are an antibody, an antibody fragment, an scFv, a dsFv, a Fv, a Fab, an scFab, a (Fab′)2, a single domain antibody (SDAB), a VH or VL domain, or a camelid VHH domain.

12-21. (canceled)

22. The MBM of claim 1, wherein the component of a human TCR complex is CD3.

23. (canceled)

24. The MBM of claim 22, wherein ABM3 is an anti-CD3 antibody, an antibody fragment, an scFv, a dsFv, a Fv, a Fab, an scFab, a (Fab′)2, a single domain antibody (SDAB), a VH or VL domain, or a camelid VHH domain.

25-38. (canceled)

39. The MBM of claim 1, which is a trivalent MBM, optionally wherein the trivalent MBM has any one of the configurations depicted in FIGS. 1B-1U.

40. The MBM of claim 1, which is a tetravalent MBM, optionally wherein the tetravalent MBM has any one of the configurations depicted in FIGS. 1P-1R.

41. The MBM of claim 1, which is a pentavalent MBM, optionally wherein the pentavalent MBM has the configuration depicted in FIG. 1S.

42. The MBM of claim 1, which is a hexavalent MBM, optionally wherein the hexavalent MBM has the configuration depicted in FIG. 1T or FIG. 1U.

43. (canceled)

44. A conjugate comprising the MBM of claim 1 and a cytotoxic or cytostatic agent.

45. A pharmaceutical composition comprising the MBM of claim 1 and a pharmaceutically acceptable excipient.

46. A method of treating a subject with a B cell malignancy, comprising administering to a subject suffering from the B cell malignancy an effective amount of the MBM of claim 1.

47. (canceled)

48. (canceled)

49. The method of claim 46, wherein the B cell malignancy is Hodgkin's lymphoma, non-Hodgkin's lymphoma or multiple myeloma.

50. (canceled)

51. A method of treating a subject with an autoimmune disorder, comprising administering to a subject diagnosed with an autoimmune disorder an effective amount of the MBM of claim 1.

52. The method of claim 51, wherein the autoimmune disorder is systemic lupus erythematosus (SLE), Sjögren's syndrome, scleroderma, rheumatoid arthritis (RA), juvenile idiopathic arthritis, graft versus host disease, dermatomyositis, type I diabetes mellitus, Hashimoto's thyroiditis, Graves's disease, Addison's disease, celiac disease, Crohn's Disease, pernicious anaemia, pemphigus vulgaris, vitiligo, autoimmune haemolytic anaemia, idiopathic thrombocytopenic purpura, giant cell arteritis, myasthenia gravis, multiple sclerosis (MS) (e.g., relapsing-remitting MS (RRMS)), glomerulonephritis, Goodpasture's syndrome, bullous pemphigoid, colitis ulcerosa, Guillain-Barré syndrome, chronic inflammatory demyelinating polyneuropathy, anti-phospholipid syndrome, narcolepsy, sarcoidosis, or Wegener's granulomatosis.

53. A nucleic acid or plurality of nucleic acids encoding the MBM of claim 1.

54. A cell engineered to express the MBM of claim 1.

55. A cell transfected with one or more expression vectors comprising one or more nucleic acid sequences encoding the MBM of claim 1 under the control of one or more promoters.

56. A method of producing a MBM, comprising:
(a) culturing the cell of claim 54 in conditions under which the MBM is expressed; and
(b) recovering the MBM from the cell culture.
